(19) United States
(12) Patent Application Publication
(10) Pub. No.: US 2025/0127918 A1
(43) Pub. Date: Apr. 24, 2025

(54) SITE-SELECTIVE CONJUGATION OF A PHARMACEUTICAL AGENT TO AN ANTIBODY USING AN AFFINITY PEPTIDE

(71) Applicant: Massachusetts Institute of Technology, Cambridge, MA (US)

(72) Inventors: Bradley L. Pentelute, Revere, MA (US); Andrei Ioan Loas, Boston, MA (US); Jacob Rodriguez, Brighton, MA (US); Heemal Dhanjee, Winnetka, CA (US); Aurelie Rondon, Cambridge, MA (US); Katsushi Kitahara, Ikeda (JP)

(73) Assignee: Massachusetts Institute of Technology, Cambridge, MA (US)

(21) Appl. No.: 18/904,022

(22) Filed: Oct. 1, 2024

Related U.S. Application Data
(60) Provisional application No. 63/587,137, filed on Oct. 1, 2023.

(51) Int. Cl. A61K 47/68; C07K 1/107

(52) U.S. Cl. CPC A61K 47/6889; C07K 1/1077

(57) ABSTRACT

The present disclosure provides site-selective conjugation of a pharmaceutical agent to an antibody using an affinity peptide. The antibody-pharmaceutical agent conjugates may be useful in treating, preventing, or diagnosing diseases in subjects.

### RELATED APPLICATION

The present application claims the benefit of priority under 35 U.S.C. § 119 (e) to U.S. Provisional Application No. 63/587,137, filed Oct. 1, 2023, the entire contents of which are incorporated herein by reference.

### REFERENCE TO AN ELECTRONIC SEQUENCE LISTING

The content of the electronic sequence listing (M065670540US01-SEQ-WWZ.xml; Size: 49,265 bytes; and Date of Creation: Sep. 30, 2024) is herein incorporated by reference in its entirety.

### BACKGROUND

Peptides are highly selective biopolymers, usually able to bind to specific cell receptors, or ion channels, to trigger intracellular effects. They are also excellent vectors, capable of transporting cargos to specific targets, making them highly suitable for precision medicine as peptide-drug conjugates. Compared to other biologics, peptides are easier to produce, cheaper, and result in lower immunogenicity and enhanced tissue penetration (B. M. Cooper, J. Iegre, D. H. O′ Donovan, M. Ölwegård Halvarsson, D. R. Spring, Peptides as a platform for targeted therapeutics for cancer: peptide-drug conjugates (PDCs). Chem. Soc. Rev. 50, 1480-1494 (2021)). However, peptide therapeutics usually make poor drugs due to a low chemical and physical stability and very short plasmatic half-life, resulting in their renal elimination in a few minutes. Last decades efforts thereby focused on the extension of peptide pharmacokinetic (PK) and pharmacodynamic (PD) properties following chemical (or chemoenzymatic) modifications, to slow-down renal clearance (A. Rondon, S. Mahri, F. Morales-Yanez, M. Dumoulin, R. Vanbever, Protein engineering strategies for improved pharmacokinetics. Advanced Functional Materials. 31, 2101633 (2021)), increase chemical and physical stability (H. E. Blackwell, R. H. Grubbs, Highly efficient synthesis of covalently cross-linked peptide helices by ring-closing metathesis. Angewandte Chemie International Edition. 37, 3281-3284 (1998), C. Heinis, T. Rutherford, S. Freund, G. Winter, Phage-encoded combinatorial chemical libraries based on bicyclic peptides. Nat Chem Biol. 5, 502-507 (2009)), and enhance bioavailability (D. J. Drucker, Advances in oral peptide therapeutics. Nat Rev Drug Discov. 19, 277-289 (2020)). Today, despite about 80 peptide drugs being already approved worldwide, and a hundred more currently in clinical trials, the drug market still faces a major challenge in designing and producing long-acting peptide therapeutics.

### SUMMARY OF THE DISCLOSURE

The present disclosure provides site-selective conjugation of a pharmaceutical agent to an antibody using an affinity peptide. In one aspect, the present disclosure provides a first modified affinity peptide, wherein n1 instances of the amino acid residues of the affinity peptide are independently modified with a moiety of Formula A′:

-L¹-E²-L²-(E³)ₙ₃ (A′).

In certain embodiments, each instance of E² is independently —O—C(═O)—, —O—S(═O)—, —O—S(═O)₂—, —O—C(═O)—NR^{a}—, —O—S(═O)—NR^{a}—, —O—S(═O)₂—NR^{a}—. In certain embodiments, each instance of E³ is independently a first reactive moiety.

In another aspect, the present disclosure provides a second modified affinity peptide, wherein n1 instances of the amino acid residues of the affinity peptide are independently modified with a moiety of Formula B′:

-L¹-E²-L²-(E³⁴-L³-M)ₙ₃ (B′).

In certain embodiments, each instance of M is independently a radical of a pharmaceutical agent or absent.

In another aspect, the present disclosure provides an antibody-pharmaceutical agent conjugate, wherein n2 instances of the lysine residues of the antibody are independently modified with a moiety of Formula C′:

—(CH₂)₄-E¹²-L²-(E³⁴-L³-M)ₙ₃ (C′).

In another aspect, the present disclosure provides a composition comprising:
a first modified affinity peptide, second modified affinity peptide, or antibody-pharmaceutical agent conjugate; and
optionally one or more excipients.

In another aspect, the present disclosure provides a kit comprising:
a first modified affinity peptide, second modified affinity peptide, antibody-pharmaceutical agent conjugate, or composition; and
instructions for using the first modified affinity peptide, second modified affinity peptide, antibody-pharmaceutical agent conjugate, or composition.

The first modified affinity peptide may be useful in preparing the second modified affinity peptide. The second modified affinity peptide (e.g., the affinity peptide moiety of the second modified affinity peptide) may bind the antibody (e.g., the heavy chain of the antibody). E² of the second modified affinity peptide may get close to, and as a result, react with, a lysine residue of the antibody. The reaction may occur under physiological conditions. The reaction may occur in a subject (e.g., a human). An antibody-pharmaceutical agent conjugate may form after the reaction. The antibody-pharmaceutical agent conjugate may not comprise the affinity peptide moiety.

The antibody-pharmaceutical agent conjugate may be useful for delivering the pharmaceutical agent to a subject, cell, tissue, or biological sample. In other aspects, the present disclosure provides a method comprising administering to a subject in need thereof an effective amount of a first modified affinity peptide, a second modified affinity peptide, an antibody-pharmaceutical agent conjugate, or a composition. In another aspect, the present disclosure provides a method comprising contacting a cell, tissue, or biological sample with a first modified affinity peptide, second modified affinity peptide, antibody-pharmaceutical agent conjugate, or a composition. In certain embodiments, the subject is in need of treatment, prevention, or diagnosis of a disease. The antibody-pharmaceutical agent conjugate may be useful for treating, preventing, or diagnosing a disease in a subject in need thereof. The antibody-pharmaceutical agent conjugate may be advantageous over the pharmaceutical agent because the former may show higher distribution, potency, efficacy, bioavailability, safety, and/or subject compliance; lower clearance; wider therapeutic window; fewer and/or less severe side effects; and/or lower toxicity and/or resistance to treatment than the latter.

In one embodiment of any one of the peptides or conjugates provided herein, the distance between an electrophile and a target of interest is about 10 Angstroms.

In one embodiment of any one of the peptides or conjugates provided herein, the administration is in vitro or in vivo. In one embodiment of any one of the peptides or conjugates provided herein, the “painting” or conjugation to a target occurs in vivo after the administration of the any one of the peptides or conjugates provided herein.

The details of one or more embodiments of the disclosure are set forth herein. Other features, objects, and advantages of the disclosure will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds (e.g., the first modified affinity peptides, second modified affinity peptides, antibody-pharmaceutical agent conjugates) described herein can comprise one or more asymmetric centers, and thus can exist in various stereoisomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. In some embodiments, isomers are isolated from mixtures by methods known to those skilled in the art, including chiral high pressure liquid chromatography (HPLC) and the formation and crystallization of chiral salts; or preferred isomers are prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The disclosure additionally encompasses compounds as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 200 carbon atoms (“C₁₋₂₀₀ alkyl”). In some embodiments, an alkyl group has 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as fluorine). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “heteroalkyl” refers to an alkyl group, which further includes at least one heteroatom (e.g., 1, 2, 3, or 4 heteroatoms) selected from oxygen, nitrogen, sulfur, and phosphorous within (e.g., inserted between adjacent carbon atoms of) and/or placed at one or more terminal position(s) of the parent chain. In certain embodiments, a heteroalkyl group refers to a saturated group having from 1 to 200 carbon atoms and 1 or more heteroatoms within the parent chain (“heteroC₁₋₂₀₀ alkyl”). In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents (e.g., oxo, substituted or unsubstituted C₁₋₆ alkyl (e.g., —CH₃)). In certain embodiments, the heteroalkyl group is an unsubstituted heteroC₁₋₁₂ alkyl. In certain embodiments, the heteroalkyl group is a substituted heteroC₁₋₁₂ alkyl. In some embodiments, unsubstituted heteroC₁ alkyl is —OCH₃ or —CH₂OH. In some embodiments, substituted heteroC₁ alkyl is —C(═O)NH₂. In some embodiments, unsubstituted heteroC₂ alkyl is —OCH₂CH₃, —CH₂OCH₃, or —CH₂CH₂OH. The terms “heteroC_{z1-z2} alkyl” and “C_{z1-z2} heteroalkyl” are used interchangeably, wherein each of z1 and z2 is independently an integer.

The term “alkenyl” refers to a radical of a straight-chain or branched hydrocarbon group having from 1 to 200 carbon atoms and one or more carbon-carbon double bonds (e.g., 1, 2, 3, or 4 double bonds). In some embodiments, an alkenyl group has 1 to 200 carbon atoms (“C₁₋₂₀₀ alkenyl”). In some embodiments, an alkenyl group has at least 2 carbon atoms. In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some 26 embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some 28 embodiments, an alkenyl group has 1 carbon atom (“C₁ alkenyl”). In certain embodiments, an alkenyl group is C₂₋₃ alkenyl, C₂₋₄ alkenyl, C₂₋₅ alkenyl, C₂₋₆ alkenyl, C₂₋₇ alkenyl, C₂₋₈ alkenyl, C₂₋₉ alkenyl, C₂₋₁₀ alkenyl, C₂₋₁₂ alkenyl, C₂₋₁₆ alkenyl, C₂₋₂₀ alkenyl, C₂₋₃₀ alkenyl, C₂₋₄₀ alkenyl, C₂₋₅₀ alkenyl, C₂₋₆₀ alkenyl, C₂₋₇₀ alkenyl, C₂₋₈₀ alkenyl, C₂₋₉₀ alkenyl, or C₂₋₁₀₀ alkenyl. The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

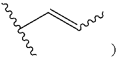

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, sulfur, and phosphorous within (e.g., inserted between adjacent carbon atoms of) and/or placed at one or more terminal position(s) of the parent chain. In certain embodiments, a heteroalkenyl group refers to a group having from 1 to 200 carbon atoms, at least one double bond, and 1 or more heteroatoms within the parent chain (“heteroC₁₋₂₀₀ alkenyl”). In some embodiments, a heteroalkenyl group has at least 2 carbon atoms. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). In certain embodiments, a heteroalkenyl group is C₂₋₃ heteroalkenyl, C₂₋₄ heteroalkenyl, C₂₋₅ heteroalkenyl, C₂₋₆ heteroalkenyl, C₂₋₇ heteroalkenyl, C₂₋₈ heteroalkenyl, C₂₋₉ heteroalkenyl, C₂₋₁₀ heteroalkenyl, C₂₋₁₂ heteroalkenyl, C₂₋₁₆ heteroalkenyl, C₂₋₂₀ heteroalkenyl, C₂₋₃₀ heteroalkenyl, C₂₋₄₀ heteroalkenyl, C₂₋₅₀ heteroalkenyl, C₂₋₆₀ heteroalkenyl, C₂₋₇₀ heteroalkenyl, C₂₋₈₀ heteroalkenyl, C₂₋₉₀ heteroalkenyl, or C₂₋₁₀₀ heteroalkenyl. Unless otherwise specified, each instance of a heteroalkenyl group is independently unsubstituted (an “unsubstituted heteroalkenyl”) or substituted (a “substituted heteroalkenyl”) with one or more substituents (e.g., oxo, substituted or unsubstituted C₁₋₆ alkyl (e.g., —CH₃)). In certain embodiments, the heteroalkenyl group is an unsubstituted heteroC₁₋₂₀ alkenyl. In certain embodiments, the heteroalkenyl group is a substituted heteroC₁₋₂₀ alkenyl. In some embodiments, unsubstituted heteroC₁ alkenyl is —CH═NH or ═N—CH₃. The terms “heteroC_{z1-z2} alkenyl” and “C_{z1-z2} heteroalkenyl” are used interchangeably, wherein each of z1 and z2 is independently an integer.

The term “alkynyl” refers to a radical of a straight-chain or branched hydrocarbon group having from 1 to 200 carbon atoms and one or more carbon-carbon triple bonds (e.g., 1, 2, 3, or 4 triple bonds) (“C₁₋₂₀₀ alkynyl”). In some embodiments, an alkynyl group has 1 to 20 carbon atoms (“C₁₋₂₀ alkynyl”). In some embodiments, an alkynyl group has at least 2 carbon atoms. In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). In certain embodiments, an alkynyl group is C₂₋₃ alkynyl, C₂₋₄ alkynyl, C₂₋₅ alkynyl, C₂₋₆ alkynyl, C₂₋₇ alkynyl, C₂₋₈ alkynyl, C₂₋₉ alkynyl, C₂₋₁₀ alkynyl, C₂₋₁₂ alkynyl, C₂₋₁₆ alkynyl, C₂₋₂₀ alkynyl, C₂₋₃₀ alkynyl, C₂₋₄₀ alkynyl, C₂₋₅₀ alkynyl, C₂₋₆₀ alkynyl, C₂₋₇₀ alkynyl, C₂₋₈₀ alkynyl, C₂₋₉₀ alkynyl, or C₂₋₁₀₀ alkynyl. The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, sulfur, and phosphorous within (e.g., inserted between adjacent carbon atoms of) and/or placed at one or more terminal position(s) of the parent chain. In certain embodiments, a heteroalkynyl group refers to a group having from 1 to 200 carbon atoms, at least one triple bond, and 1 or more heteroatoms within the parent chain (“heteroC₁₋₂₀₀ alkynyl”). In some embodiments, a heteroalkynyl group has at least 2 carbon atoms. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). In certain embodiments, a heteroalkynyl group is C₂₋₃ heteroalkynyl, C₂₋₄ heteroalkynyl, C₂₋₅ heteroalkynyl, C₂₋₆ heteroalkynyl, C₂₋₇ heteroalkynyl, C₂₋₈ heteroalkynyl, C₂₋₉ heteroalkynyl, C₂₋₁₀ heteroalkynyl, C₂₋₁₂ heteroalkynyl, C₂₋₁₆ heteroalkynyl, C₂₋₂₀ heteroalkynyl, C₂₋₃₀ heteroalkynyl, C₂₋₄₀ heteroalkynyl, C₂₋₅₀ heteroalkynyl, C₂₋₆₀ heteroalkynyl, C₂₋₇₀ heteroalkynyl, C₂₋₈₀ heteroalkynyl, C₂₋₉₀ heteroalkynyl, or C₂₋₁₀₀ heteroalkynyl. Unless otherwise specified, each instance of a heteroalkynyl group is independently unsubstituted (an “unsubstituted heteroalkynyl”) or substituted (a “substituted heteroalkynyl”) with one or more substituents (e.g., oxo, substituted or unsubstituted C₁₋₆ alkyl (e.g., —CH₃)). In certain embodiments, the heteroalkynyl group is an unsubstituted heteroC₁₋₂₀ alkynyl. In certain embodiments, the heteroalkynyl group is a substituted heteroC₁₋₂₀ alkynyl. In some embodiments, unsubstituted heteroC₁ alkynyl is —C═N. The terms “heteroC_{z1-z2} alkynyl” and “C_{z1-z2} heteroalkynyl” are used interchangeably, wherein each of z1 and z2 is independently an integer.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and is saturated or contains one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In some embodiments, in heterocyclyl groups that contain one or more nitrogen atoms, the point of attachment is a carbon or nitrogen atom, as valency permits. In some embodiments, a heterocyclyl group is either monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and is either saturated or contains one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring 12 systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In some embodiments, in heteroaryl groups that contain one or more nitrogen atoms, the point of attachment is a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. In some embodiments, in polycyclic heteroaryl groups wherein one ring does not contain a heteroatom (e.g., indolyl, quinolinyl, carbazolyl, and the like) the point of attachment is on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each 24 instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a polyvalent (e.g., divalent, trivalent, tetravalent) moiety as valency permits, e.g., alkylene is the polyvalent moiety of alkyl, alkenylene is the polyvalent moiety of alkenyl, alkynylene is the polyvalent moiety of alkynyl, heteroalkylene is the polyvalent moiety of heteroalkyl, heteroalkenylene is the polyvalent moiety of heteroalkenyl, heteroalkynylene is the polyvalent moiety of heteroalkynyl, carbocyclylene is the polyvalent moiety of carbocyclyl, heterocyclylene is the polyvalent moiety of heterocyclyl, arylene is the polyvalent moiety of aryl, and heteroarylene is the polyvalent moiety of heteroaryl. In certain embodiments, affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl. In certain embodiments, the length of a polyvalent moiety is the smallest number of backbone atoms between any two attachment points.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₁₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₁₀ alkyl, C₁-perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl) C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents are joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR)₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and Rec are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{aa}, —C(═O)OR^{aa}, —C(═O)—O—C(═O)R^{aa}, —C(═O)SR^{aa}, —C(═O)N(R^{bb})₂, —C(═S)R^{aa}, —C(═S)N(R^{bb})₂, and —C(═S)S(R^{aa}), —C(═NR^{bb})R^{aa}, —C(═NR^{bb})OR^{aa}, —C(═NR^{bb})SR^{aa}, and —C(═NR^{bb})N(R^{bb})₂, wherein R^{aa} and R^{bb} are as defined herein. In some embodiments, the term “acyl” refers to a group having the general formula-C(═O)R^{aa}, —C(═O)OR^{aa}, —C(═O)—O—C(═O)R^{aa}, —C(═O)SR^{aa}, or —C(═O)N(R^{bb})₂.

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

Nitrogen atoms are substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R_{cc})₂, —SO₂R_{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methoxy, methoxylmethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl) ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio)ethyl carbonate (Peoc), 16 isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl) phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. In some embodiments, an anionic counterion is monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF⁶₋, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, as valency permits (e.g., from 1 to 4, from 1 to 3, from 1 to 2, from 2 to 4, from 2 to 3, or from 3 to 4 instances, inclusive). In certain embodiments, “at least one instance” refers to each instance. When “at least one instance” refers to at least two instances, the at least two instances may be the same as, or different from, each other.

The term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “leaving group” is given its ordinary meaning in the art of synthetic organic chemistry and refers to an atom or a group capable of being displaced by a nucleophile. Examples of suitable leaving groups include halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other examples of leaving groups are water, ammonia, alcohols, ether moieties, thioether moieties, zinc halides, magnesium moieties, diazonium salts, and copper moieties.

The term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than 2,000 g/mol. In certain embodiments, the molecular weight of a small molecule is not more than 1,500 g/mol. In certain embodiments, the molecular weight of a small molecule is not more than 1,000 g/mol, not more than 900 g/mol, not more than 800 g/mol, not more than 700 g/mol, not more than 600 g/mol, not more than 500 g/mol, not more than 400 g/mol, not more than 300 g/mol, not more than 200 g/mol, or not more than 100 g/mol. In certain embodiments, the molecular weight of a small molecule is at least 100 g/mol, at least 200 g/mol, at least 300 g/mol, at least 400 g/mol, at least 500 g/mol, at least 600 g/mol, at least 700 g/mol, at least 800 g/mol, or at least 900 g/mol, or at least 1,000 g/mol. Combinations of the above ranges (e.g., at least 200 g/mol and not more than 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460; drugs for veterinary use are listed by the FDA under 21 C.F.R. §§ 500 through 589. All listed drugs are considered acceptable for use in accordance with the present disclosure.

The term “peptide,” “polypeptide,” or “protein” refers to an oligomer or polymer of amino acid residues covalently connected together by peptide bonds. A peptide, polypeptide, or protein may be of any size, structure, and function, and may be an individual peptide, polypeptide, or protein, or a collection (e.g., a complex) of peptides, polypeptides, and proteins, and optionally small molecules and/or metal ions. In certain embodiments, a peptide comprises between 2 and 10, between 11 and 20, between 21 and 30, between 31 and 40, or between 41 and 50, inclusive, amino acid residues. In certain embodiments, a polypeptide or protein comprises between 51 and 100, between 101 and 200, between 201 and 300, between 301 and 500, between 501 and 1,000, between 1,001 and 3,000, between 3,001 and 10,000, or between 10,001 and 30,000, inclusive, amino acid residues. A peptide, polypeptide, or protein may contain only natural amino acids but no non-natural amino acids; only non-natural amino acids but no natural amino acids; or both natural and non-natural amino acids. A peptide, polypeptide, or protein may contain amino acid analogs only or in addition to natural and/or non-natural amino acids. In certain embodiments, the amino acid residues of a peptide, polypeptide, or protein are residues of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and/or valine, in D and/or L form (e.g., in L form). One or more of the amino acid residues in a peptide, polypeptide, or protein may be alpha amino acid residues or homologs thereof (e.g., beta amino acid residues). One or more of the amino acid residues in a peptide, polypeptide, or protein May be protected or unprotected. One or more (e.g., two) of the termini of a peptide, polypeptide, or protein may be protected (e.g., to form an ester or amide) or unprotected (e.g., as —NH₂, —NH₃⁺, —C(═O) OH, or —C(═O)O⁻). One or more of the amino acid residues in a peptide, polypeptide, or protein may be modified or unmodified. A modification to an amino acid residue in a peptide, polypeptide, or protein may be an addition of a carbohydrate group, a hydroxyl group, a phosphate group, a farnesyl group, an isofarnesyl group, a fatty acid group, or a linker for conjugation or functionalization. A peptide, polypeptide, or protein may be naturally occurring, recombinant, synthetic, or a combination thereof. A peptide, polypeptide, or protein may be a fragment of a naturally occurring peptide, polypeptide, or protein.

The term “affinity peptide” refers to a peptide that binds preferentially to an antibody than to another protein. The affinity peptide may bind preferentially to the heavy chain of the antibody than to a light chain of the antibody. The preferential binding may be at least 2-fold, at least 5-fold, at least 10-fold, at least 30-fold, at least 100-fold, at least 1000-fold, or at least 10000-fold, inclusive, preferential binding. The binding may be under physiological conditions. The affinity peptide may be an unmodified peptide. The affinity peptide moiety of a modified affinity peptide is the affinity peptide radical without the modification.

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonuculeotide may comprise a modified base moiety which is selected from the group including 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or lncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. In some embodiments, the non-human animal is a male or female at any stage of development. In some embodiments, the non-human animal is a transgenic animal or genetically engineered animal. The term “patient” refers to a human subject in need of treatment of a disease.

The term “tissue” refers to any biological tissue of a subject (including a group of cells, a body part, or an organ) or a part thereof, including blood and/or lymph vessels. In some embodiments, “tissue” is the object to which a compound, and/or composition of the disclosure is delivered. In some embodiments, a tissue is an abnormal or unhealthy tissue, which may need to be treated. A tissue may also be a normal or healthy tissue that is under a higher than normal risk of becoming abnormal or unhealthy, which may need to be prevented.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound provided herein, a compound useful in a provided method, or a pharmaceutical composition provided herein, in or on a subject.

The terms “condition,” “disease,” and “disorder” are used interchangeably.

The terms “treatment,” “treat,” and “treating” refer to reversing, alleviating, delaying the onset of, or inhibiting the progress of a disease described herein. In some embodiments, treatment is administered after one or more signs or symptoms of the disease have developed or have been observed. In other embodiments, treatment is administered in the absence of signs or symptoms of the disease. For example, in some embodiments, treatment is administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting the activity and/or production of a GSK3. In certain embodiments, a therapeutically effective amount is an amount sufficient for treating a disease. In certain embodiments, a therapeutically effective amount is an amount sufficient for inhibiting the activity and/or production of a GSK3 and treating a disease.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibiting the activity and/or production of a GSK3. In certain embodiments, a prophylactically effective amount is an amount sufficient for preventing a disease. In certain embodiments, a prophylactically effective amount is an amount sufficient for inhibiting the activity and/or production of a GSK3 and preventing a disease.

The term “genetic disease” refers to a disease caused by one or more abnormalities in the genome of a subject, such as a disease that is present from birth of the subject. Genetic diseases may be heritable and may be passed down from the parents' genes. A genetic disease may also be caused by mutations or changes of the DNAs and/or RNAs of the subject. In such cases, the genetic disease will be heritable if it occurs in the germline. Exemplary genetic diseases include Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Friedreich's ataxia, Gaucher's disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Werner syndrome, Williams syndrome, Wilson's disease, xeroderma piginentosum, and Zellweger syndrome.

A “proliferative disease” refers to a disease that occurs due to abnormal growth or extension by the multiplication of cells (Walker, Cambridge Dictionary of Biology, Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is 10 located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See, e.g., Stedman's Medical Dictionary, 25th ed.; Hensyl ed.; Williams & Wilkins: Philadelphia, 1990. The cancer may be a solid tumor. The cancer may be a hematological malignancy. Exemplary cancers include acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous 16 panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, dermatitis (e.g., stasis dermatitis, allergic contact dermatitis, atopic dermatitis, irritant contact dermatitis, neurodermatitis perioral dermatitis, seborrheic dermatitis), hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, necrotizing enterocolitis, inflammatory rosacea. An ocular inflammatory disease includes post-surgical inflammation.

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barré syndrome, Hashimoto's thyroiditis, and cardiomyopathy.

A “hematological disease” includes a disease which affects a hematopoietic cell or tissue. Hematological diseases include diseases associated with aberrant hematological content and/or function. Examples of hematological diseases include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia (AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopeniaurpura and disseminated intravascular coagulation, infections by parasites such as Plasmodium, chemical injuries from, e.g., lead poisoning, and hypersplenism.

The term “neurological disease” refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including Alzheimer's disease, Parkinson's disease, amyotrophic lateral sclerosis, tauopathies (including frontotemporal dementia), and Huntington's disease. Examples of neurological diseases include headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness, include bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

A “painful condition” includes neuropathic pain (e.g., peripheral neuropathic pain), central pain, deafferentiation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawl symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

The term “metabolic disease” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include the endocrine (hormonal) control system (e.g., the insulin pathway, the enteroendocrine hormones including GLP-1, PYY or the like), the neural control system (e.g., GLP-1 in the brain), or the like. Examples of metabolic disorders include diabetes (e.g., Type I diabetes, Type II diabetes, gestational diabetes), hyperglycemia, hyperinsulinemia, insulin resistance, and obesity.

The term “psychiatric disorder” refers to a condition or disorder relating to the functioning of the brain and the cognitive processes or behavior. Psychiatric disorders may be further classified based on the type of neurological disturbance affecting the mental faculties. Psychiatric disorders are expressed primarily in abnormalities of thought, feeling, emotion, and/or behavior producing either distress or impairment of function (for example, impairment of mental function such with dementia or senility). The term “psychiatric disorder” is, accordingly, sometimes used interchangeably with the term “mental disorder” or the term “mental illness”. A psychiatric disorder is often characterized by a psychological or behavioral pattern that occurs in an individual and is thought to cause distress or disability that is not expected as part of normal development or culture. Definitions, assessments, and classifications of mental disorders can vary, but guideline criteria listed in the International Classification of Diseases and Related Health Problems (ICD, published by the World Health Organization, WHO), or the Diagnostic and Statistical Manual of Mental Disorders (DSM, published by the American Psychiatric Association, APA) and other manuals are widely accepted by mental health professionals. Individuals may be evaluated for various psychiatric disorders using criteria set forth in these and other publications accepted by medical practitioners in the field and the manifestation and severity of a psychiatric disorder may be determined in an individual using these publications. Categories of diagnoses in these schemes may include dissociative disorders, mood disorders, anxiety disorders, psychotic disorders, eating disorders, developmental disorders, personality disorders, and other categories. There are different categories of mental disorder, and many different facets of human behavior and personality that can become disordered. One group of psychiatric disorders includes disorders of thinking and cognition, such as schizophrenia and delirium. A second group of psychiatric disorders includes disorders of mood, such as affective disorders and anxiety. A third group of psychiatric disorders includes disorders of social behavior, such as character defects and personality disorders. And a fourth group of psychiatric disorders includes disorders of learning, memory, and intelligence, such as mental retardation and dementia. Accordingly, psychiatric disorders encompass schizophrenia, delirium, attention deficit disorder (ADD), schizoaffective disorder, depression (e.g., lithium-resistant depression), mania, attention deficit disorders, drug addiction, dementia, agitation, apathy, anxiety, psychoses, personality disorders, bipolar disorders, unipolar affective disorder, obsessive-compulsive disorders, eating disorders, post-traumatic stress disorders, irritability, adolescent conduct disorder and disinhibition.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows the general concept of IgG-binder electrophile peptides conjugated GLP1 drug. DAR: antibody-drug ratio; GLP1: glucagon-like peptide 1; PD: pharmacodynamic; PK: pharmacokinetic.

FIGS. 2A to 2D demonstrate the in vitro and in vivo azido transfer to IgGs. FIG. 2A shows the in vitro azido transfer to mIgG following 2-, 6-, and 24-hours of incubation in mouse sera. FIG. 2B shows the experimental design for assessing the azido transfer in mice via subcutaneous (SC) and intraperitoneal (IP) injection. FIG. 2C shows the percentage of azido transfer to mIgG relative to total mIgG detected via ELISA following subcutaneous injection of 30 mg/kg of Z33-E20Hcy or PEG₈-Z33-E20HCy. FIG. 2D shows the percentage of azido transfer to mIgG relative to total mIgG detected via ELISA following subcutaneous or intraperitoneal injection of 10 or 30 mg/kg of Z33-E20Hcy or PEG₈-Z33-E20Hcy.

FIGS. 3A to 3D illustrates the utility of IgG painting using GLP2 drugs for improving drug PK/PD. FIG. 3A shows the experimental design of the in vivo GLP1 transfer to mIgG in WT Swiss mice. FIG. 3B shows the body weight follow-up after a single injection of GLP1 analogs. FIG. 3C shows the blood glucose concentration curve (top) obtained after the intraperitoneal (IP) injection of 20% dextrose (Ip-GTT) and their respective area under the curve (AUC, bottom) at 24-, 72-, and 144-hours post-subcutaneous (SC) injection of 10 mg/kg of GLP1 analogs. GLP-01: GLP1 peptide; GLP-03: Z33-E20HCy-GLP1; GLP-04: PEG₈-Z33-E20HCy-GLP1. FIG. 3D shows the experimental design for assessing the in vivo GLP1 transfer to mIgG in obese mice.

FIGS. 4A to 4J show IgG painting with radionuclides following different routes of injection. FIG. 4A shows the experimental design in which about 1 MBq of Z33-P16p conjugated to deferoxamine B (DFO) and radiolabeled with Zr-89, 1 MBq pf Z33-E20HCy conjugated to DFO and radiolabeled with Zr-89, and PEG₈-Z33-E20HCy conjugated to DFO and radiolabeled with Zr-89 was injected into WT Swiss mice either intravenously (IV), intraperitoneally (IP), or subcutaneously (SC). FIG. 4B shows whole body biodistribution of [⁸⁹Zr]Zr-Z33-P16p, [⁸⁹Zr]Zr-Z33-E20Hcy, and [⁸⁹Zr]Zr-PEG₈-Z33-E20Hcy at 144-hours 30 post-IV injection. FIG. 4C compares the injected activity per gram (% IA/g) of [⁸⁹Zr]Zr-Z33-P16p, [⁸⁹Zr]Zr-Z33-E20Hcy, and [⁸⁹Zr]Zr-PEG8-Z33-E20Hcy in the liver, lungs, and kidneys at 144 hours post-IV injection. FIG. 4D shows PET imaging and uptake of [⁸⁹Zr]Zr-Z33-P16p, [⁸⁹Zr]Zr-Z33-E20Hcy, and [⁸⁹Zr]Zr-PEG8-Z33-E20Hcy in the spleen at 144 hours post-IV injection. FIG. 4E shows PET-CT imaging of [⁸⁹Zr]Zr-Z33-P16p, [⁸⁹Zr]Zr-Z33-E20Hcy, and [⁸⁹Zr]Zr-PEG8-Z33-E20Hcy at 24- and 144-hours post-IV injection. FIG. 4F shows whole-body biodistribution of [⁸⁹Zr]Zr-Z33-P16p and [⁸⁹Zr]Zr-Z33-E20Hcy at 2-, 24-, 72-, and 144-hours post-IP injection. FIG. 4G compares the injected activity per gram (% IA/g) of [⁸⁹Zr]Zr-Z33-P16p and [⁸⁹Zr]Zr-Z33-E20Hcy in the spleen, pancreas, kidneys, liver, and stomach at 24- and 144-hours post-IP injection. FIG. 4H shows the whole body biodistribution of [⁸⁹Zr]Zr-Z33-E20Hcy at 2-, 24-, 72-, 144-, and 240-hours post-SC injection. FIG. 4I compares the whole body biodistribution of [⁸⁹Zr]Zr-Z33-E20Hcy at 144 hours post-IV, post-IP, and post-SC injection. FIG. 4J compares the uptake of [⁸⁹Zr]Zr-Z33-E20Hcy in the spleen, liver, pancreas, and kidneys at 144 hours post-IV, post-IP, and post-SC injection.

FIGS. 5A to 5B show the mAB binding peptide Z33 and its cysteine variants. FIG. 5A shows the co-crystal structure of mAB-Fc and protein A. Key lysine residues are indicated. FIG. 5B shows the structure of Z33. Seven possible cysteine variants are indicated in bold.

FIG. 6 is a schematic of the study design. Lysines on IgG and the binding peptide Z33 are shown. Binding peptides containing cysteine variants and palladium oxidative addition complexes are prepared in tandem. Electrophile-attached IgG binding peptides are then prepared by C-S arylation for selective IgG modification.

FIG. 7 shows the design and synthesis of an aryl carbamate attached Z33 by C-S arylation.

FIGS. 8A to 8C show the modification of trastuzumab. FIG. 8A shows the reaction setup using trastuzumab as the IgG under basic pH conditions. FIG. 8B is a schematic of the procedure for analyzing the IgG by mass spectrometry (MS) using de-glycosylation and disulfide cleavage reactions. FIG. 8C shows the mass spectrometry results of trastuzumab (top) and trastuzumab following de-glycosylation and disulfide cleavage reactions (bottom).

FIGS. 9A to 9C show modifications of trastuzumab using a different electrophiles. FIG. 9A shows the design and synthesis of the Z33-homocysteine electrophile. FIG. 9B shows the structures and mass spectrometry analysis of para- and meta-substituted electrophile using the Z33-homocysteine electrophile. FIG. 9C shows the structure and mass spectrometry analysis of para- and meta-substituted phenyl ester.

FIG. 10 shows the structure and liquid chromatography-mass spectrometry analysis of the meta-substituted phenyl ester.

FIGS. 11A to 11C show the structures and mass spectrometry analysis of the light chains (right column) and heavy chains (left column) of unreacted (top row) and meta-substituted phenyl ester-modified (bottom row) IgG1 (FIG. 11A), IgG2 (FIG. 11B), and IgG4 (FIG. 11C).

FIGS. 12A to 12B show the peptide mapping results. FIG. 12A shows the sequence analysis. Highlighted fragments were detected using Orbitrap LC-MS/MS. FIG. 12B shows the peak area of a heavy chain and light chain lysine evaluated using Orbitrap LC-MS/MS.

FIG. 13 shows the structure and mass spectrometry analysis after reacting tetrazine as a bioconjugation handle at room temperature (top), at 37° C. (middle), and in two portions at 37° C. (bottom).

FIGS. 14A to 14C show a selective antibody-drug conjugate preparation for in vitro cytotoxicity experiments. FIG. 14A is a schematic of the reaction for the preparation of the antibody-drug conjugate. FIG. 14B is a mass spectrometry analysis of the light chain (left) and heavy chain (right) after the first step of the reaction. FIG. 14C shows size exclusion chromatography purification (left) profile and liquid chromatography-mass spectrometry analysis of the light chain (middle) and heavy chain (right) of the final ADC product.

FIGS. 15A to 15C show the result of an electrophilic Z33 reagent with a longer linker. FIG. 15A shows the co-crystal structure of IgG and an electrophile attached Z33 variant and the reaction conditions. FIG. 15B shows the structure of the cysteine variant of Z33 (top), the homocysteine variant of Z33 (middle), and the cysteine variant of Z33 with a meta-substituted electrophile (bottom). FIG. 15C shows mass spectrometry analysis of the heavy chain for the cysteine variant of Z33 (top), the homocysteine variant of Z33 (second from top), the cysteine variant of Z33 with a meta-substituted electrophile (third from top), and the cysteine variant of Z33 with a meta-substituted electrophile when the reaction was performed at 37° C. (bottom). FIG. 15D shows the mass spectrometry analysis of the light chain (middle) and heavy chain (right) of the cysteine variant of Z33 with a meta-substituted electrophile when the reaction was performed at 37° C.

FIGS. 16A to 16D show a reaction schematic and mass spectrometry results of a double modification on a single IgG. FIG. 16A shows the sequential reaction of IgG with two Z33 variants to achieve K248 and K317 modification on the same IgG. FIG. 16B shows the mass spectrometry analysis of the light chain (left) and heavy chain (right) of the native IgG. FIG. 16C shows the mass spectrometry analysis of the light chain (left) and heavy chain (right) of the IgG after the first reaction and subsequent modification of K317. FIG. 16D shows the mass spectrometry analysis of the light chain (left) and heavy chain (right) of the IgG after the second modification and subsequent reaction of K248.

FIGS. 17A to 17B show the result of an electrophilic Z33 reagent with a longer linker. FIG. 17A shows the co-crystal structure of IgG and an electrophile attached Z33 variant. FIG. 17B shows the structures and mass spectrometry analysis of the heavy chain of the Z33-R31 cysteine variant (top), the Z33-D32 cysteine variant (middle), and Z33-D32 homocysteine variant (bottom).

FIG. 18 shows the conversion ratio of the heavy chain as a function of time in PBS.

FIG. 19 shows mass spectrometry analysis of the light chain (left) and heavy chain (right) of a control mouse IgG (top row) and of mouse IgG after reaction with a Z33 variant (bottom row).

FIG. 20 is a reaction schematic for the preparation of the nucleophilic Z33 variants.

FIG. 21 shows the structures, total ion current chromatograms, and mass spectrometry analysis of seven nucleophilic Z33 variants.

FIGS. 22A to 22C show the results of Z33 variant screening. FIG. 22A shows the reaction schematic for the Z33 variant screening, as well as an exemplary structure of the Z33-E20HCy variant. FIG. 22B shows gel electrophoresis results of the reaction of trastuzumab with each of the seven Z33-cysteine variants. FIG. 22C shows the schematic, structures, and gel electrophoresis results of the reaction of trastuzumab with Z33-E20Hcy containing a meta-SO₂F electrophile (E1), para-SO₂F (E2), and a previously disclosed electrophile (E3).

FIGS. 23A to 23B show the results of azide group incorporation on Z33-E20HCy. FIG. 23A shows the reaction schematic and gel electrophoresis results of reacting azide-attached Z33-E20HCy with trastuzumab. FIG. 23B shows the mass spectrometry analysis of trastuzumab (top) and the products of reacting azide-attached Z33-E20HCy with trastuzumab (bottom).

FIGS. 24A to 24C show schematics and gel electrophoresis results of a one-pot reaction. FIG. 24A show the reaction of Z33-E20HCy to produce the electrophilic Z33, which can then be reacted with trastuzumab. FIG. 24B shows the reaction product of trastuzumab with the electrophilic Z33 in 100 mM phosphate and 10% DMF. FIG. 24C shows the reaction product of trastuzumab with the electrophilic Z33 in 100 mM HEPES and 200 mM NaCl.

FIG. 25 shows a schematic of two approaches for trastuzumab modification to produce a cross-linked protein (top) or for small compound installation using a mAB binder kick-out strategy (bottom).

FIG. 26 shows the reagent design for small compound installation using a mAB binder kick-out strategy. The reagent is designed by flipping the carbamate of the protein crosslinking reagent.

FIG. 27 shows the reaction schematic and mass spectrometry results of the light chain (left) and heavy chain (right) of trastuzumab (top row) and trastuzumab reacted with a Z33-Hcy electrophile using a binder kick-out reagent (bottom row).

FIG. 28 shows the reaction schematic and mass spectrometry results of the light chain (left) and heavy chain (right) of trastuzumab (top row), trastuzumab reacted with a Z33-Hcy variant with a meta-substituted electrophile (second from top), trastuzumab reacted with a Z33-Hcy variant with a para-substituted electrophile (second from bottom), and trastuzumab reacted with a 5-azido-pentanoic acid-3-phenylester reagent (bottom).

FIG. 29 shows mass spectrometry analysis of electrophile screening on Z33-E20Hcy.

FIGS. 30A to 30B show small compound conjugation via an azide group. FIG. 30A shows the reaction schematic of modified-trastuzumab with DBCO. FIG. 30B shows gel electrophoresis results of trastuzumab (Tmab) and modified-trastuzumab (mod.T-mab) in the presence of absence of fluorescently-labelled DBCO (DBCO-Fl). The bar plot shows the relative fluorescence intensity of the light chain (LC) and heavy chain (HC) after reaction of modified-trastuzumab with fluorescently-labelled DBCO.

FIG. 31 shows mass spectrometry results of the light chain (left) and heavy chain (right) IgG modified by 5-azido-pentanoic acid-3-phenylester.

FIG. 32 shows structures of Z33 variants and their corresponding heavy chain conversion efficiencies after reaction with IgG.

FIG. 33 shows a reaction schematic and mass spectrometry analysis of a Z33 electrophile reacted with trastuzumab or RNase A.

FIGS. 34A to 34C show mass spectrometry analysis of a control antibody or a Z33-electrophile reacted with the antibody. FIG. 34A shows mass spectrometry results of human IgG2 (top) and human IgG2 reacted with a Z33-electrophile (bottom). FIG. 34B shows mass spectrometry results of human IgG4 (top) and human IgG4 reacted with a Z33-electrophile (bottom). FIG. 34C shows mass spectrometry results of mouse IgG1 (top) and mouse IgG1 reacted with a Z33-electrophile (bottom).

FIGS. 35A to 35E show in vitro azido transfer to mouse IgG using Z33-electrophiles. FIG. 35A shows the in-house sandwich ELISA setup used to determine the percentage of azido transfer to mouse IgG. FIG. 35B shows the reactivity comparison between Z33-P16p-N₃, Z33-E20Hcy-N₃, and PEG₈-Z33-E20Hcy-N₃ after reaction with mouse IgG for 2-, 6-, or 24-hours. FIG. 35C shows the experimental design used to compare percentage of azido transfer using subcutaneous (SC) and intraperitoneal (IP) injection. FIG. 35D shows the percentage azido transfer to mouse IgG evaluated 2 hours after subcutaneous injection of Z33-E20Hcy-N₃ and PEG₈-Z33-E20Hcy-N₃. FIG. 35E shows the percentage azido transfer to mouse IgG of 10 mg/kg or 30 mg/kg of a Z33-electrophile evaluated 2 hours after subcutaneous or intraperitoneal injection.

FIGS. 36A to 36H demonstrate whole-body distribution of Z33-P16p-E20C-N₃ and Z33-E20-Hcy-N₃ in mice. FIG. 36A shows a reaction schematic used to radiolabel Z33 peptides. FIG. 36B shows the purity of radiolabeling Z33-P16p-E20C-N₃ with Zr-89. FIG. 36C shows the purity of radiolabeling and Z33-E20-Hcy-N₃ with Zr-89. FIG. 36D shows the experimental design to determine the biodistribution of radiolabeled-Z33 peptides in mice. FIG. 36E shows the injected activity per gram (% IA/g) of [⁸⁹Zr]Zr-Z33-P16p in mouse organs 2-, 24-, 72-, and 144-hours post-intraperitoneal injection. shows the injected activity per gram (% IA/g) of [⁸⁹Zr]Zr-Z33-P16p in mouse organs 2-, 24-, 72-, and 144-hours post-intraperitoneal injection. FIG. 36F shows the injected activity per gram (% IA/g) of [⁸⁹Zr]Zr-Z33-E20Hcy in mouse organs 2-, 24-, 72-, and 144-hours post-intraperitoneal injection. FIG. 36G compares the injected activity per gram (% IA/g) of [⁸⁹Zr]Zr-Z33-P16p and [⁸⁹Zr]Zr-Z33-E20Hcy in the spleen, pancreas, kidneys, liver, and stomach 2 hours post-intraperitoneal injection. FIG. 36H compares the injected activity per gram (% IA/g) of [⁸⁹Zr]Zr-Z33-P16p and [⁸⁹Zr]Zr-Z33-E20Hcy in the spleen, pancreas, kidneys, liver, and stomach 144 hours post-intraperitoneal injection.

FIG. 37 provides an overview of using Z33 peptides to increase the PK/PD of GLP1.

FIG. 38 shows a reaction schematic for the preparation of a GLP1 analog transfer reagent.

FIG. 39 shows HPLC analysis of the GLP1-transfer reagent.

FIGS. 40A to 40B show the reactivity analysis of the GLP1 transfer reagent. FIG. 40A shows the reaction schematic of the GLP1 transfer reagent with trastuzumab. FIG. 40B shows mass spectrometry analysis of the light chain (left) and heavy chain (right) of trastuzumab after reacting with the GLP1 transfer reagent.

FIG. 41 shows an experimental design to evaluate Z33-GLP1 peptide transfer in mice.

FIGS. 42A to 42C show the structure of GLP1 drugs semaglutide (FIG. 42A), GLP1 peptide alone (FIG. 42B), and Z33-E20Hcy-GLP1 (FIG. 42C) and their toxicity in mice.

FIG. 43 shows the impact of Z33-GLP1 transfer reagents on body weight loss over time after a single injection of PBS, semaglutide, GLP1, Z33-E20Hcy-GLP1, and PEG₈-E20Hcy-GLP1.

FIGS. 44A to 44B show the results of intraperitoneal-glucose tolerance tests (Ip-GTT) hours (FIG. 44A) or 144 hours (FIG. 44B) after injection of PBS, semaglutide, GLP1, Z33-E20Hcy-GLP1, and PEG₈-E20Hcy-GLP1. The bar plots indicate the area under the curve of the blood glucose concentration after 120 minutes.

FIG. 45 shows an exemplary SDS-PAGE result for determining the reaction conversion of IgGs.

FIGS. 46A to 46E show ADC mass analysis results for the synthesized electrophile-attached Z33 reagents.

FIG. 47 shows LC-MS analysis of the solution obtained from the large scale reaction for entry 15.

FIG. 48 shows SDS-PAGE analysis of the solution obtained from the large scale reaction for entry 15.

FIG. 49 shows LC-MS results of trastuzumab (top), entry 6 (middle), and a mixture of trastuzumab and entry 6 (bottom).

FIGS. 50A to 50E shows LC-MS results of electrophile-attached Z33E20/Hcy reagents.

FIG. 51 shows LC-MS results of electrophile-attached Z33M3C/Hcy reagents.

FIG. 52 shows LC-MS results of electrophile-attached Z33R31C/Hcy and D32C/Hcy reagents.

FIGS. 53A to 53D shows LC-MS results of small bioconjugation handle installation to other IgGs.

FIGS. 54A to 54B show the sequence analysis of two modified trastuzumab and peak areas of the observed modified peptides derived from LC-MS/MS analysis.

FIG. 55 shows HPLC analysis of two exemplary antibody-drug conjugates at each step of the preparation: modified trastuzumab (top row), the intermediate structure (middle row), and the final reacted sample (bottom row).

FIG. 56 shows HPLC analysis of an exemplary antibody-drug conjugate at the intermediate stage (top) and the final reacted sample (bottom).

FIGS. 57A to 57C show the sequence, structure, and LC-MS analysis of the fractions obtained after reaction of GLP-01 with DBCO-PEG24-NHS.

FIGS. 58A to 58B show the LC-MS analysis of GLP-03.

FIGS. 59A to 59B shows the LC-MS analysis of GLP-04.

FIG. 60 shows a generic reaction schematic for the conjugation of Z33 analog to trastuzumab.

FIG. 61 shows a generic reaction schematic for the conjugation of Z33E20Hcy reagents to trastuzumab.

FIG. 62 shows a generic reaction schematic for the small bioconjugation handle installation to trastuzumab using the electrophile-attached Z33M3C/Hcy reagents.

FIG. 63 shows a generic reaction schematic for the small bioconjugation handle installation to trastuzumab using the electrophile-attached Z33R31C/Hcy and D32C/Hcy Reagents.

FIG. 64 shows a generic reaction schematic for small bioconjugation handle installation to other IgGs.

FIG. 65 shows the reaction schematic for GLP-03 synthesis.

FIG. 66 shows the reaction schematic for GLP-04 synthesis.

FIG. 67 shows the reaction schematic for GLP1 transfer to trastuzumab.

FIG. 68 shows the sequence and structural element of the tirzepatide peptide backbone.

FIG. 69 shows a reaction schematic of the preparation of the tirzepatide-transfer reagent.

FIGS. 70A to 70C show characterization of the tirzepatide-transfer reagent. FIG. 70A shows the structure of the tirzepatide-transfer reagent. FIG. 70B shows the LC-MS analysis of the tirzepatide-transfer reagent. FIG. 70C shows the ULPC analysis of the purified tirzepatide-transfer reagent.

FIGS. 71A to 71B show reactivity of the tirzepatide-transfer reagent against human IgG trastuzumab. FIG. 71A shows a schematic of the reaction of the tirzepatide-transfer reagent with human IgG under three different conditions: 1) 100 mM HEPES pH 8.5 at room temperature for 24 hours; 2) 100 mM HEPES pH 8.5 at 37° C. for 24 hours; and, 3) PBS at 37° C. for 24 hours. FIG. 71B shows gel electrophoresis analysis of trastuzumab alone and after reaction with the tirzepatide-transfer reagent under each of the three conditions.

FIG. 72 shows a generic reaction schematic for obtaining a pure tirzepatide-transfer reagent.

FIGS. 73A and 73B show a general overview of the in vivo IgG ‘painting’ technology. FIG. 73A shows a schematic illustrating a concept. FIG. 73B shows an in vivo reaction to native IgGs. Z33 peptide variant conjugated to electrophile-drug moieties is administered in vivo. The peptide recognizes lysine-317 of the IgG Fc-domain heavy chain A enabling the transfer of its payload to IgGs through proximity effect. The reaction is fast (a few hours), biocompatible and covalent. The binding of IgGs to the neonatal Fc receptor (FcRn) the half-life is expected to extend and enhance drug efficacy. This technology is versatile and can be accomplished with a variety of cargoes: small molecules, therapeutic peptides, or even radionuclides. DAR: drug-antibody ratio; DBCO: dibenzyl-cyclooctyne; Hcy: homocysteine; IgG: immunoglobulin G; IP: intraperitoneal; IV: intravenous; PEG: polyethylene glycol; SC: subcutaneous.

FIGS. 74A to 74D show in vivo IgG painting extends GLP-1a pharmacodynamic profile in WT Swiss mice. FIG. 74A shows structures of the synthesized compounds 2a-3a. FIG. 74B shows in vivo experimental design: female WT Swiss mice were SC injected with 10 mg/kg (˜100 nmol) of semaglutide or with 10 mg/kg (˜25 nmol) of 2a or 3a. FIG. 74C shows body weight change from Day 0. FIG. 74D shows Ip-GTT curves (top) and AUC (bottom) at 24 h, 72 h, and 144 h post drug injection. Statistical analysis was performed using two-way ANOVA (FIG. 74C), multiparametric T-tests (FIG. 74C), or non-parametric T-tests (FIG. 74D): *P<0.05, **P<0.01, ***P<0.001, ****P<0.0001. AUC: area under the curve; GTT: glucose-tolerance tests; IP: intraperitoneal; SC: subcutaneous.

FIGS. 75A to 75G show in vivo IgG painting with 2a,2b sustains body weight loss and exerts extended blood glucose control in obese Lep^{ob/ob} mice. FIG. 75A shows structure of compounds 2a-3a. FIG. 75B shows experimental design: Lep^{ob/ob} obese mice (n=6 per compound, split as 3 males and 3 females) were SC injected with 10 mg/kg (˜170 nmol) of semaglutide or 10 mg/kg (˜45 nmol) of 2a,3a. Non-obese C57BL/6J mice (n=6, 3 males and 20 females) have also been included in the study as an additional naïve control to obtain the baseline values for healthy subjects. FIG. 75C shows body weight change from Day 0. FIG. 75D shows blood glucose measured after 6 hours of fasting. FIG. 75E shows Ip-GTT curves (top) and AUC (bottom) at 24 h and 144 h p.i. FIG. 75F shows Ip-ITT curve (left) and AUC 0-90 min (right) at 240 h p.i. FIG. 75G shows plasma glucose disappearance rate (k-ITT) determined at 240 h p.i. Statistical analysis was performed using two-way ANOVA (FIG. 75C), multiparametric T-tests (FIG. 75C), or non-parametric T-tests (FIGS. 75D to 75G): *P<0.05, **P<0.01, ***P<0.001, ****P<0.0001. AUC: area under the curve; GTT: glucose tolerance test; IP: intraperitoneal; ITT: insulin tolerance test; p.i: post injection, SC: subcutaneous.

FIGS. 76A to 76F show payload versatility highlighted by in vivo radionuclide transfer to native mIgGs in WT Swiss mice. FIG. 76A shows radiolabeling of peptides 1-3. FIG. 76B shows experimental design: 1.2-1.5 MBq of [⁸⁹Zr]Zr-1-3 was administered IV in female WT Swiss mice (n=4 mice per compound). Longitudinal PET-CT imaging was performed over 6 days, and organs were harvested for quantification. FIG. 76C shows whole-body biodistribution of [⁸⁹Zr]Zr-1-3 at 144 h p.i. FIG. 76D shows representative longitudinal PET-CT imaging at 2 h (top), 24 h (bottom), and 144 h (bottom) p.i. Scale (top): min=1.0e+05 kBq/mL, max=3.0e+06 kBq/mL; (bottom): min=2.7e+04 kBq/mL, max=4.0e+05 kBq/mL. The same thresholds have been applied for the PET of the free [⁸⁹Zr]Zr used as control. FIG. 76E shows PET-CT of all spleens and uptake at 144 h p.i. Scale: min=1.31e+04 kBq/mL, max=3.73e+05 kBq/mL. FIG. 76F shows a comparison of the % IA/g in the liver, lungs, and kidneys, at 144 h p.i. Statistical analysis was performed using non-8 parametric T-tests: *P<0.05, **P<0.01, ***P<0.001, ****P<0.0001. % IA/g: percent of injected activity per gram of tissue; IV: intravenous; PET-CT: positron emission tomography-computed tomography scan; p.i.: post injection.

FIG. 77 shows the co-crystal structure of the human IgG Fc fragment and the B domain of Protein A (PDB: 1FC2). Human Fc fragment, B domain of protein A, K317 of the human IgG Fc fragment, and E20 of the B domain of protein A are shown.

FIGS. 78A and 78B show synthesis of the electrophile affinity peptides 1-6. FIG. 78A shows variants of the minimized Z-domain of protein A (Z33 peptide) were synthesized using automated fast-flow peptide synthesis (AFPS). FIG. 78B shows conjugation of the electrophile to the Z33 peptide variants 1-6 to obtain N3-1-6 via an arylation reaction using palladium reagent.

FIGS. 79A and 79B show in vitro reactivity of selected electrophile affinity peptides for human IgG1 Trastuzumab (Tmab). FIG. 79A shows percentage of modification on heavy and light chains of Tmab depending on the amino acid sequence of the affinity peptide and structure of the electrophiles. The conjugation site on the affinity peptide is highlighted in grey. “Heavy chain reaction selectivity” was calculated based on the percentage of lysine residues on the heavy chain with at least one modification. FIG. 79B shows mass spectra (MS) post reaction. Tmab was deglycosylated and reduced beforehand. Left: MS for the light chain; Right: MS for the heavy chain.

FIGS. 80A and 80B show identification of the sites of modification on IgG1 Trastuzumab (Tmab) after in vitro reaction with compound 2. FIG. 80A shows sequencing of Tmab after digestion with trypsin and analysis by LC-MS/MS. Lysines are underlined. Gray bars below the sequence indicate the observed fragments. The lysines exhibiting modifications are boxed. FIG. 80B shows peak areas corresponding to the lysines modified by the affinity peptides.

FIGS. 81A to 81D show in vitro reactivity of compound 2 for other IgG subclasses and species. FIG. 81A shows “heavy chain reaction selectivity” was calculated based on the percentage of lysine residues with at least one modification. FIGS. 81B to 81D show mass spectra (MS) post reaction. IgGs were deglycosylated and reduced beforehand. Left: MS for the light chain; Right: MS for the heavy chain.

FIGS. 82A to 82C show in vitro selectivity of compound 2 for IgG1 FIG. 82A shows compound 2 incubation with Tmab and RNAse A FIG. 82B shows mass spectrometry of native RNase A FIG. 82C shows mass spectrometry after reaction with compound 2. Data were obtained after glycan removal and disulfide reduction of Tmab. The top panel shows the mass of RNase A, and the bottom panel shows the mass of Tmab heavy chains. Native RNase A and the mass obtained after reaction with compound 2 are shown.

FIGS. 83A to 83F show in vitro and in vivo azido transfer to mouse IgG (mIgG) using electrophile affinity peptides N₃-1-6. FIG. 83A shows sequences and structures of electrophile affinity peptide N₃-1-6. FIG. 83B shows ELISA assays E1 and E² developed for the quantification of azido transfer to native mIgG. FIG. 83C shows in vitro azido transfer to mIgG using electrophile affinity peptides N3-1-6 quantified by E1/E2 after 2, 6, or 24 h of incubation (˜4 nmol of N₃-1-6) in mouse sera. FIG. 83D shows in vitro azido transfer to mIgG using peptides electrophile affinity peptides N₃-1-3 quantified by E1/E2 after 2, 6, or 24 h of incubation in mouse sera (˜4 nmol of N₃-1-6). Data show mean±SD from 6-7 replicates over 3 independent experiments. Statistical analysis was performed using non-parametric T-tests. *P<0.05, **P<0.01, ***P<0.001, ****P<0.0001. FIG. 83E shows in vivo azido transfer to mIgG in mice sera quantified by E1/E² at 24 h after subcutaneous (SC) injection of 30 mg/kg (˜280 nmol) of electrophile affinity peptides N₃-2,3 in female WT Swiss mice. FIG. 83F shows in vivo azido transfer to mIgG in mice sera quantified by E1/E2 at 24 h after either SC or intraperitoneal (IP) injection of 10 mg/kg (˜90 nmol) or 30 mg/kg (˜280 nmol) of electrophile affinity peptides N₃-2,3 in female WT Swiss mice.

FIGS. 84A and 84B show synthesis of GLP-1 conjugates. FIG. 84A shows Semaglutide, Liraglutide, and Tirzepatide peptide backbones were synthesized using automated fast-flow peptide synthesis (AFPS). Semaglutide and Liraglutide were then conjugated to dibenzyl cyclooctyne (DBCO)-PEG₂₃ moiety to obtain DBCO-GLP-1-a,b reagents. Tirzepatide was conjugated to bifunctional DBCO-PEG₄-tetrazine (Tz) to obtain reagent Tz-GLP-1c. FIG. 84B shows synthesis of electrophile affinity peptide 2c. Affinity peptide 2 was first conjugated to DBCO-PEG₁₂-transcyclooctene, then conjugated to Tz-PEG₄-GLP1c through the inverse-electron demand Diels-Alder click cycloaddition to obtain reagent 2c.

FIG. 85 shows analyzed sequences of GLP-1a and conjugated click dibenzylcyclooctyne moiety. The indicated fragments were analyzed using LC/MS-MS to confirm the site of modification.

FIGS. 86A to 86C show in vitro reactivity of compounds 2a and 3a for human IgG1 Trastuzumab (Tmab). FIG. 86A shows a scheme of the reaction FIG. 86B shows SDS-PAGE result post reaction with corresponding quantification. Tmab was reduced beforehand. FIG. 86C shows mass spectra (MS) post reaction. Tmab was deglycosylated and reduced beforehand.

FIGS. 87A and 87B show in vitro reactivity of compound 2b for human IgG1 Trastuzumab (Tmab). FIG. 87A shows a scheme of the reaction FIG. 87B shows SDS-PAGE result post reaction and corresponding quantification. Tmab was reduced beforehand.

FIGS. 88A and 88B show reactivity of compound 2c for human IgG1 Trastuzumab (Tmab). FIG. 88A shows a scheme of the reaction. FIG. 88B shows SDS-PAGE result post reaction and corresponding quantification. Tmab was reduced beforehand.

FIGS. 89A to 89D show dose response of semaglutide in wild-type Swiss mice. FIG. 89A shows a structure of commercial semaglutide. FIG. 89B shows experimental design: female WT Swiss mice were subcutaneously (SC) injected with either 0.5 mg/kg (˜5 nmol), 3 mg/kg (˜30 nmol), or 10 mg/kg (˜100 nmol) of semaglutide. Intraperitoneal glucose tolerance test (Ip-GTT) was performed after 24 h. FIG. 89C shows body weight change from Day 0. FIG. 89D shows Ip-GTT curve (left) and area under the curve (AUC) from 0 to 120 min (right) performed at 24 h post Semaglutide injection. Statistical analysis was performed using non-parametric T-tests: *P<0.05, **P<0.01, ***P<0.001, ****P<0.0001.

FIGS. 90A to 90C show dose response of 2a. FIG. 90A shows structure of the affinity peptide 2a. FIG. 90B shows experimental design: female WT Swiss mice were subcutaneously (SC) injected either with one single injection of 10 mg/kg (25 nmol) or 30 mg/kg (75 nmol) of 2a, or with a stacking dose of 3 injections of 10 mg/kg (75 nmol total) through 1 injection per week for 3 weeks. FIG. 90C shows intraperitoneal glucose tolerance tests (Ip-GTT) performed at 24 h, 72 h, and 144 h post 2a injection. Top: Ip-GTT curves; Bottom: area under the curves (AUC) from 0 to 120 min. For the stacking dose (3×10 mg/kg), Ip-GTT were performed after the 3rd injection of 2a. Statistical analysis was performed using non-parametric T-tests: *P<0.05, **P<0.01, ***P<0.001, ****P<0.0001.

FIGS. 91A to 91D show complementary data set for fasting blood glucose in WT Swiss mice and Lep^{ob/ob} mice. FIGS. 74A to 75G and FIGS. 87A to 89D. FIG. 91A shows structures of compounds 2a,3a. FIG. 91B shows a comparison of fasting blood glucose, after 6 hours of fasting, in different female mice strains. FIG. 91C shows fasting blood glucose in female WT Swiss mice at 24 h, 72 h, and 144 h post SC injection of 10 mg/kg (˜100 nmol) of Semaglutide (Ozempic®), or 10 mg/kg (˜25 nmol) of compound 2a,3a. FIG. 91D shows fasting blood glucose in Lep^{ob/ob} mice at 144 h post injection of 10 mg/kg (˜170 nmol) of Semaglutide (Ozempic®), or 10 mg/kg (˜45 nmol) of compound 2a,3a following SC route (left) or IP (right).

FIGS. 92A to 92E show IgG painting with GLP-1a analogs in Lep^{ob/ob} mice after intraperitoneal (IP) injection of a single 10 mg/kg dose extends GLP-1 efficacy. FIG. 92A shows structures of electrophile affinity peptides 2a,3a. FIG. 92B shows Experimental design: male and female Lep^{ob/ob} mice were IP injected with 10 mg/kg (˜170 nmol) of either Semaglutide (Ozempic®) or 10 mg/kg (˜45 nmol) of 2a,3a. IP-glucose tolerance tests (Ip-GTT) was performed after 24 h and 144 h, and Ip-insulin tolerance test (Ip-ITT) was performed at 240 post injection. FIG. 92C shows Ip-GTT curves (top) and area under the curve AUC0-120 min (Bottom) measured at 24 h and 144 h post drug injection. FIG. 92D shows Ip-ITT curve (left) and AUC0-90 min (right) measured at 240 h post drug injection. FIG. 92E shows Plasma glucose rate disappearance (K-ITT) determined from Ip-ITT data, at 240 h post drug injection.

FIGS. 93A to 93E show complementary data set for IgG painting with radionuclides after intravenous (IV) injection. Complete data set from FIGS. 76A-76F. FIG. 93A shows structures of the radiolabeled affinity peptides [⁸⁹Zr]Zr-1-3. FIG. 93B shows experimental design: female WT Swiss mice were IV injected with 1.2-1.5 MBq of [⁸⁹Zr]Zr-1-3. PET-CT imaging was performed over 6 days, then the organs were harvested for quantification. FIG. 93C shows Whole-body biodistribution of [⁸⁹Zr]Zr-1-3 at 144 h post injection. FIG. 93D shows uptake in the bones at 144 h post injection. FIG. 93E shows PET-CT imaging at the 72-h time-point post injection. Statistical analysis was performed using non-parametric T-tests: *P<0.05, **P<0.01, ***P<0.001, ****P<0.0001.

FIGS. 94A to 94D show in vivo IgG painting with radionuclides after subcutaneous (SC) injection. FIG. 94A shows structures of the radiolabeled affinity peptides [⁸⁹Zr]Zr-2,3. FIG. 94B shows experimental design: female WT Swiss mice were SC injected with 1.2-1.5 MBq of peptides [⁸⁹Zr]Zr-2,3 then sacrificed at different time points to quantify the uptake in the organs. FIG. 94C shows whole-body biodistribution of [⁸⁹Zr]Zr-2 (left) and [⁸⁹Zr]Zr-3 (right). FIG. 94D shows whole-body biodistribution of free [⁸⁹Zr]Zr at 240 h post injection (˜1 MBq).

FIGS. 95A to 95H show in vivo IgG painting with radionuclides after intraperitoneal (IP) injection. FIG. 95A shows structures of the radiolabeled affinity peptides [⁸⁹Zr]Zr-1,2. FIG. 95B shows experimental design: female WT Swiss mice were IP injected with 1.2-1.5 MBq of [⁸⁹Zr]Zr-1,2 then sacrificed at different time points to quantify the uptake in the organs. FIG. 95C shows whole-body biodistribution of [⁸⁹Zr]Zr-1 (top left), [⁸⁹Zr]Zr-2 (top right), and whole-body biodistribution of free [⁸⁹Zr]Zr at 144 h post injection (˜1 MBq) (bottom. FIG. 95D shows uptake in the bones. FIG. 95E shows percent of injected activity per gram (% IA/g) in the spleen. FIG. 95F shows % IA/g in the pancreas. FIG. 95G shows % IA/g in the liver. FIG. 95H shows % IA/g in the kidneys. Statistical analysis performed using non-parametric T-tests: *P<0.05, **P<0.01, ***P<0.001, ****P<0.0001.

FIGS. 96A to 96D show a comparison of the routes of injection for in vivo IgG painting using radionuclides, in WT Swiss mice. FIG. 96A shows structures of the radiolabeled affinity peptides [⁸⁹Zr]Zr-2,3. FIG. 96B shows experimental design: female WT Swiss mice were injected, either IV, IP, or SC, with 1.2-1.5 MBq of [⁸⁹Zr]Zr-2,3. FIG. 96C shows whole-body biodistribution of [⁸⁹Zr]Zr-2 (left) and [⁸⁹Zr]Zr-3 (right) following IV, Ip, or SC, at 144 h post injection. FIG. 96D shows whole-body biodistribution of free [⁸⁹Zr]Zr at h following IV, IP, or SC injection (˜1 MBq).

FIGS. 97A and 97B show in vitro reactivity of selected electrophile affinity peptides for human IgG1 Trastuzumab (Tmab). FIG. 97A shows percentage of modification on heavy and light chains of Tmab depending on the amino acid sequence of the affinity peptide and structure of the electrophiles. The conjugation site on the affinity peptide is highlighted in gray. “Heavy chain reaction selectivity” was calculated based on the percentage of lysine residues on the heavy chain with at least one modification. FIG. 97B shows mass spectra (MS) post reaction. Tmab was deglycosylated and reduced beforehand. Left: MS for the light chain; Right: MS for the heavy chain.

FIGS. 98A and 98B show identification of the sites of modification on IgG1 Trastuzumab (Tmab) after reaction with N₃-8-VI. FIG. 98A shows sequencing of Tmab after digestion with trypsin and analysis by LC-MS/MS. Lysines are underlined. Gray bars below the sequence indicate the observed fragments. The lysines exhibiting modifications are boxed. FIG. 98B shows peak areas corresponding to the lysines modified by the affinity peptides.

FIGS. 99A and 99B show in vitro reactivity of selected electrophile affinity peptides for human IgG1 Trastuzumab (Tmab). FIG. 99A shows percentage of modification on heavy and light chains of Tmab depending on the amino acid sequence of the affinity peptide and structure of the electrophiles. The conjugation site on the affinity peptide is highlighted in gray. “Heavy chain reaction selectivity” was calculated based on the percentage of lysine residues on the heavy chain with at least one modification. FIG. 99B shows mass spectra (MS) post reaction. Tmab was deglycosylated and reduced beforehand. Left: MS for the light chain; Right: MS for the heavy chain.

FIG. 100 shows dual modification of Trastuzumab heavy chain by stepwise addition of compound 2 and N₃-8-VI.

FIGS. 101A to 101D show dual in vivo IgG painting with radiolabeled GLP-1a analogs after SC injection. FIG. 101A shows synthesis and radiolabeling of [⁸⁹Zr]Zr-12,13. FIG. 101B shows synthesis and radiolabeling of [⁸⁹Zr]Zr-GLP-1a. FIG. 101C shows experimental design: female WT Swiss mice were SC injected with 1.2-1.5 MBq of [⁸⁹Zr]Zr-12a, 13a or [⁸⁹Zr]Zr-GLP-1a then sacrificed at several time points post injection. FIGS. 101D to 101E show whole-body biodistribution of [⁸⁹Zr]Zr-GLP-1a (FIG. 101D), [⁸⁹Zr]Zr-12a (FIG. 101E, left), and [⁸⁹Zr]Zr-13a (FIG. 101E, right).

FIGS. 102A and 102B show in vitro reactivity of MMAE-attached electrophile affinity peptides for human IgG1 Trastuzumab (Tmab). FIG. 102A shows percentage of modification on heavy and light chains of Tmab depending on the amino acid sequence of the 20 affinity peptide and structure of the electrophiles. The conjugation site on the affinity peptide is highlighted in gray. “Heavy chain reaction selectivity” was calculated based on the percentage of lysine residues on the heavy chain with at least one modification. FIG. 102B shows mass spectra (MS) post reaction. Tmab was deglycosylated and reduced beforehand.

FIG. 103 shows a structure of peptide 1. LC-MS analysis: Method A; Calculated Mass: 4078.6 Da; Observed Mass: 4078.6 Da.

FIG. 104 shows a structure of peptide 2. LC-MS analysis: Method D; Calculated Mass: 4092.6 Da; Observed Mass: 4093.1 Da.

FIG. 105 shows a structure of peptide 3. LC-MS analysis: Method C; Calculated Mass: 4516.1 Da; Observed Mass: 4516.2 Da.

FIG. 106 shows a structure of peptide 4. LC-MS analysis: Method E; Calculated Mass: 4092.6 Da; Observed Mass: 4092.5 Da.

FIG. 107 shows a structure of peptide 5. LC-MS analysis: Method E; Calculated Mass: 4092.6 Da; Observed Mass: 4092.5 Da.

FIG. 108 shows a structure of peptide 6. LC-MS analysis: Method E; Calculated Mass: 4092.6 Da; Observed Mass: 4092.6 Da.

FIG. 109 shows a structure of peptide BB-1. LC-MS analysis: Method D; Calculated Mass: 4078.6 Da; Observed Mass: 4078.5 Da.

FIG. 110 shows a structure of peptide BB-3. LC-MS analysis: Method A; Calculated Mass: 4090.5 Da; Observed Mass: 4090.7 Da.

FIG. 111 shows a structure of peptide BB-4. LC-MS analysis: Method E; Calculated Mass: 4051.5 Da; Observed Mass: 4052.3 Da.

FIG. 112 shows a structure of peptide BB-5. LC-MS analysis: Method A; Calculated Mass: 4092.6 Da Observed Mass: 4092.7 Da.

FIG. 113 shows a structure of peptide BB-6. LC-MS analysis: Method E; Calculated Mass: 4106.6 Da; Observed Mass: 4106.5 Da.

FIG. 114 shows a structure of peptide N₃-1-I. LC-MS analysis: Method A; Calculated Mass: 4295.8 Da; Observed Mass: 4296.1 Da.

FIG. 115 shows a structure of peptide N₃-2-I. LC-MS analysis: Method A; Calculated Mass: 4309.8 Da; Observed Mass: 4310.0 Da.

FIG. 116 shows a structure of peptide N₃-3-I. LC-MS analysis: Method D; Calculated Mass: 4733.3 Da; Observed Mass: 4733.3 Da.

FIG. 117 shows a structure of peptide N₃-4-I. LC-MS analysis: Method E; Calculated Mass: 4309.8 Da; Observed Mass: 4309.7 Da.

FIG. 118 shows a structure of peptide N₃-5-I. LC-MS analysis: Method E; Calculated Mass: 4309.8 Da; Observed Mass: 4309.7 Da.

FIG. 119 shows a structure of peptide N₃-6-I. LC-MS analysis: Method E; Calculated Mass: 4309.8 Da; Observed Mass: 4309.7 Da.

FIG. 120 shows a structure of peptide BB-21. LC-MS analysis: Method E; Calculated Mass: 4345.9 Da; Observed Mass: 4345.7 Da.

FIG. 121 shows a structure of peptide BB-22. LC-MS analysis: Method E; Calculated Mass: 4309.8 Da; Observed Mass: 4309.7 Da.

FIG. 122 shows a structure of peptide BB-23. LC-MS analysis: Method E; Calculated Mass: 4334.8 Da; Observed Mass: 4334.7 Da.

FIG. 123 shows a structure of peptide N₃-12-VIII. LC-MS analysis: Method A; Calculated Mass: 4678.3 Da; Observed Mass: 4678.4 Da.

FIG. 124 shows a structure of peptide N₃-7-VI. LC-MS analysis: Method A; Calculated Mass: 4429.9 Da; Observed Mass: 4430.1 Da.

FIG. 125 shows a structure of peptide N₃-8-VI. LC-MS analysis: Method E; 2 Calculated Mass: 4443.9 Da; Observed Mass: 4443.8 Da.

FIG. 126 shows a structure of peptide N₃-7-VII. LC-MS analysis: Method E; Calculated Mass: 4429.9 Da; Observed Mass: 4430.6 Da.

FIG. 127 shows a structure of peptide N₃-8-VII. LC-MS analysis: Method E; 6 Calculated Mass: 4443.9 Da; Observed Mass: 4444.4 Da.

FIG. 128 shows a structure of peptide N₃-9-VII. LC-MS analysis: Method B; Calculated Mass: 4404.9 Da; Observed Mass: 4405.8 Da.

FIG. 129 shows a structure of peptide N₃-10-VII. LC-MS analysis: Method B; Calculated Mass: 4446.0 Da; Observed Mass: 4445.7 Da.

FIG. 130 shows a structure of peptide N₃-11-VII. LC-MS analysis: Method E; Calculated Mass: 4460.0 Da; Observed Mass: 4460.2 Da.

FIG. 131 shows a structure of peptide N₃-13-VIII. LC-MS analysis: Method A; Calculated Mass: 5101.8 Da; Observed Mass: 5102.2 Da.

FIG. 132 shows a structure of peptide 12a. LC-MS analysis*: Method B (* This data was obtained using crude reaction mixture.); Calculated Mass: 9490.7 Da; Observed Mass: 9491.0 Da.

FIG. 133 shows a structure of peptide 13a. LC-MS analysis: Method B; Calculated Mass: 9914.2 Da; Observed Mass*: 9914.7 Da (*The peak observed around 5 min elution time was also detected in the blank measurement, suggesting its origin as an unidentified compound retained in the column.)

FIG. 134 shows a structure of peptide GLP-1a. LC-MS analysis: Method D; Calculated Exact Mass: 3396.8 Da; Observed Mass: 3397.3 Da.

FIG. 135 shows a structure of peptide 2a. LC-MS analysis: Method B; Calculated Mass: 9122.3 Da; Observed Mass: 9122.7 Da.

FIG. 136 shows a structure of peptide 3a. LC-MS analysis: Method D; Calculated Mass: 9545.8 Da; Observed Mass: 9545.8 Da.

FIG. 137 shows a structure of peptide N₃-GLP-1a. LC-MS analysis: Method B; Calculated Exact Mass: 3670.1 Da; Observed Mass*: 3670.4 Da (*The peak observed around 5 min elution time was also detected in the blank measurement, suggesting its origin as an unidentified compound retained in the column.)

FIG. 138 shows a structure of peptide DBCO-PEG24-GLP-1a. LC-MS analysis: Method B; Calculated Mass: 4812.4 Da; Observed Mass: 4812.5 Da.

FIG. 139 shows a structure of peptide GLP-1b. LC-MS analysis: Method D; Calculated Mass: 3382.7 Da; Observed Mass: 3383.3 Da.

FIG. 140 shows a structure of peptide GLP-1c. LC-MS analysis: Method B; Calculated Mass: 4095.6 Da; Observed Mass: 4095.7 Da.

FIG. 141 shows a structure of peptide Z33-GLP-1c. LC-MS analysis: Method B; Calculated Mass: 10056 Da; Observed Mass: 10057 Da.

### DETAILED DESCRIPTION OF CERTAIN EMBODIMENTS OF THE DISCLOSURE

A recent success for chemical modifications leading to long-acting peptide therapeutics concerns the glucagon-like peptide-1 agonist receptors (GLP1 RAs). The incretin hormone GLP1, a 37-amino acid peptide, binds the GLP1 receptor (GLP1-R) that regulates glucogenesis by inducing glucose-dependent insulin secretion. In vivo, native GLP1 possess a very short plasmatic half-life, about few minutes, being quickly degraded through the kidneys by dipeptidyl peptidase-4 (DPP-4) and neutral endopeptidase 24.11 (NEP 24.11). However, the stability and pharmacological effects of synthetic GLP1 can be significantly improved after modifying its amino-acid sequence and conjugation to half-life extension moieties, leading to the development of several top-selling peptide drugs for the treatment of type II diabetes Mellitus (TDM) and obesity. For example, long-acting Semaglutide (Ozempic®, Wegovy®, Rybelsus®), FDA-approved in 2017, shows a 7-day half-life with a duration of efficacy of 3 days, allowing its administration once a week (refs), while Dulaglutide (Trulicity®) is covalently fused to the Fc fragment of an IgG₁, with extended drug efficacy and stability. However, the first generations of synthetic GLP1 agonists require extensive chemical or biological engineering, Fc fusion requiring humanization to reducing the risks for immunogenicity. New technologies are thereby needed to manufacture the next generation of long-acting GLP1 or other drugs.

The present disclosure provides, in one aspect, a platform to “paint” antibodies (e.g., native antibodies) with pharmaceutical agents (payloads, e.g., therapeutic agents), after in vivo administration, to use the circulating antibodies as a vehicle to extend the PK/PD of payloads. First, IgG-binder electrophile peptides derived from the structure of Z33 (an affinity peptide) were synthesized and their affinity towards human IgG1,2,4 and mouse IgG1,2a,2b investigated. It was discovered that these electrophile peptides possess a high affinity and selectivity towards IgG in mouse sera.

Further investigations using the affinity peptides highlighted the IgG-binder electrophilic peptides are capable of “painting” native antibodies (IgG1,2,4) directly inside the body of a subject, through the covalent transfer of either a small molecule, a radionuclide, or a bioactive long peptide. The transfer reaction is biocompatible, occurs without catalyzers, and/or does not require biological engineering or production of antibody-pharmaceutical agent conjugates beforehand as the payload is attached on native circulating IgGs that are already naturally produced by the organism. The ability of the antibody-pharmaceutical agent conjugates to extend the PK/PD of the pharmaceutical agent (e.g., GLP1 agonists) was successfully demonstrated up to 6 days for blood glucose control along with a body weight loss sustained for about 10 days after one single injection (vs 3 days for Semaglutide). The results show promising outcomes for making the next generation pharmaceutical agents (e.g., antidiabetic agents and antiobesity agents), and highlight the technology versatility (e.g., by enabling the transfer of not only bioactive peptides, but also potent chemicals). The antibody-pharmaceutical agent conjugates may be useful in developing of long-acting drugs.

### First Modified Affinity Peptides, Second Modified Affinity Peptides, and Antibody-Pharmaceutical Agent Conjugates

In one aspect, the present disclosure provides a first modified affinity peptide, wherein:

n1 instances of the amino acid residues of the affinity peptide are independently modified with a moiety of Formula A′:

-L¹-E²-L²-(E³)ₙ₃ (A′);
n1 is 1, 2, or 3;
each instance of L¹ is independently substituted or unsubstituted, C₁₋₁₈ heteroalkylene, substituted or unsubstituted, C₁₋₁₈ alkylene, substituted or unsubstituted, C₂₋₁₈ alkenylene, substituted or unsubstituted, C₂₋₁₈ alkynylene, substituted or unsubstituted, C₂₋₁₈ heteroalkenylene, substituted or unsubstituted, C₂₋₁₈ heteroalkynylene, substituted or unsubstituted arylene, substituted or unsubstituted carbocyclylene, substituted or unsubstituted heterocyclylene, or substituted or unsubstituted heteroarylene, optionally wherein one, two, or three backbone atoms of the C₁₋₁₈ heteroalkylene, C₁₋₁₈ alkylene, C₂₋₁₈ alkenylene, C₂₋₁₈ alkynylene, C₂₋₁₈ heteroalkenylene, or C₂₋₁₈ heteroalkynylene are independently replaced with substituted or unsubstituted arylene, substituted or unsubstituted carbocyclylene, substituted or unsubstituted heterocyclylene, or substituted or unsubstituted heteroarylene, as valency permits;
each instance of E² is independently —O—C(═O)—, —O—S(═O)—, —O—S(═O)₂—, —O—C(═O)—NR^{a}—, —O—S(═O)—NR^{a}—, —O—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of n3 is independently 1, 2, 3, or 4; and
each instance of E³ is independently a first reactive moiety.

In certain embodiments, the first modified affinity peptide is a first modified affinity peptide, wherein:
n1 instances of the amino acid residues of the affinity peptide are independently modified with a moiety of Formula A:

-L¹-E²-L²-E³ (A);
n1 is 1, 2, or 3;
each instance of L¹ is independently substituted or unsubstituted, C₁₋₁₈ heteroalkylene, substituted or unsubstituted, C₁₋₁₈ alkylene, substituted or unsubstituted, C₂₋₁₈ alkenylene, substituted or unsubstituted, C₂₋₁₈ alkynylene, substituted or unsubstituted, C₂₋₁₈ heteroalkenylene, substituted or unsubstituted, C₂₋₁₈ heteroalkynylene, substituted or unsubstituted arylene, substituted or unsubstituted carbocyclylene, substituted or unsubstituted heterocyclylene, or substituted or unsubstituted heteroarylene, optionally wherein one, two, or three backbone atoms of the C₁₋₁₈ heteroalkylene, C₁₋₁₈ alkylene, C₂₋₁₈ alkenylene, C₂₋₁₈ alkynylene, C₂₋₁₈ heteroalkenylene, or C₂₋₁₈ heteroalkynylene are independently replaced with substituted or unsubstituted arylene, substituted or unsubstituted carbocyclylene, substituted or unsubstituted heterocyclylene, or substituted or unsubstituted heteroarylene, as valency permits;
each instance of E² is independently —O—C(═O)—, —O—S(═O)—, —O—S(═O)₂—, —O—C(═O)—NR^{a}—, —O—S(═O)—NR^{a}—, —O—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits; and
each instance of E³ is independently a first reactive moiety.

In certain embodiments, Formula A′ is Formula A:

-L¹-E²-L²-E³ (A).

In another aspect, the present disclosure provides a second modified affinity peptide, wherein:
n1 instances of the amino acid residues of the affinity peptide are independently modified with a moiety of Formula B′:

-L¹-E²-L²-(E³⁴-L³-M)ₙ₃ (B′);

wherein:
n1 is 1, 2, or 3;
each instance of L¹ is independently substituted or unsubstituted, C₁₋₁₈ heteroalkylene, substituted or unsubstituted, C₁₋₁₈ alkylene, substituted or unsubstituted, C₂₋₁₈ alkenylene, substituted or unsubstituted, C₂₋₁₈ alkynylene, substituted or unsubstituted, C₂₋₁₈ heteroalkenylene, substituted or unsubstituted, C₂₋₁₈ heteroalkynylene, substituted or unsubstituted arylene, substituted or unsubstituted carbocyclylene, substituted or unsubstituted heterocyclylene, or substituted or unsubstituted heteroarylene, optionally wherein one, two, or three backbone atoms of the C₁₋₁₈ heteroalkylene, C₁₋₁₈ alkylene, C₂₋₁₈ alkenylene, C₂₋₁₈ alkynylene, C₂₋₁₈ heteroalkenylene, or C₂₋₁₈ heteroalkynylene are independently replaced with substituted or unsubstituted arylene, substituted or unsubstituted carbocyclylene, substituted or unsubstituted heterocyclylene, or substituted or unsubstituted heteroarylene, as valency permits;
each instance of E² is independently —O—C(═O)—, —O—S(═O)—, —O—S(═O)₂—, —O—C(═O)—NR^{a}—, —O—S(═O)—NR^{a}—, —O—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of n3 is independently 1, 2, 3, or 4;
each instance of E³⁴ is independently a moiety formed by reacting E³ with E⁴;
each instance of E³ is independently a first reactive moiety;
each instance of E⁴ is independently a second reactive moiety;
each instance of the first reactive moiety is capable of reacting with each instance of the second reactive moiety;
each instance of L³ is independently substituted or unsubstituted, C₁₋₂₀₀ heteroalkylene, substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkenylene, substituted or unsubstituted, C₂₋₂₀₀ heteroalkynylene, substituted or unsubstituted heterocyclylene, substituted or unsubstituted arylene, substituted or unsubstituted heteroarylene, substituted or unsubstituted carbocyclylene, or a bond, optionally wherein one or more backbone atoms of the C₁₋₂₀₀ heteroalkylene, C₁₋₂₀₀ alkylene, C₂₋₂₀₀ alkenylene, C₂₋₂₀₀ alkynylene, C₂₋₂₀₀ heteroalkenylene, or C₂₋₂₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits; and
each instance of M is independently a radical of a pharmaceutical agent or absent.

In certain embodiments, the second modified affinity peptide is a second modified affinity peptide, wherein:
n1 instances of the amino acid residues of the affinity peptide are independently modified with a moiety of Formula B:

-L¹-E²-L²-E³⁴-L³-M (B);

wherein:
n1 is 1, 2, or 3;
each instance of L¹ is independently substituted or unsubstituted, C₁₋₁₈ heteroalkylene, substituted or unsubstituted, C₁₋₁₈ alkylene, substituted or unsubstituted, C₂₋₁₈ alkenylene, substituted or unsubstituted, C₂₋₁₈ alkynylene, substituted or unsubstituted, C₂₋₁₈ heteroalkenylene, substituted or unsubstituted, C₂₋₁₈ heteroalkynylene, substituted or unsubstituted arylene, substituted or unsubstituted carbocyclylene, substituted or unsubstituted heterocyclylene, or substituted or unsubstituted heteroarylene, optionally wherein one, two, or three backbone atoms of the C₁₋₁₈ heteroalkylene, C₁₋₁₈ alkylene, C₂₋₁₈ alkenylene, C₂₋₁₈ alkynylene, C₂₋₁₈ heteroalkenylene, or C₂₋₁₈ heteroalkynylene are independently replaced with substituted or unsubstituted arylene, substituted or unsubstituted carbocyclylene, substituted or unsubstituted heterocyclylene, or substituted or unsubstituted heteroarylene, as valency permits;
each instance of E² is independently —O—C(═O)—, —O—S(═O)—, —O—S(═O)₂—, —O—C(═O)—NR^{a}—, —O—S(═O)—NR^{a}—, —O—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of E³⁴ is independently a moiety formed by reacting E³ with E⁴;
each instance of E³ is independently a first reactive moiety;
each instance of E⁴ is independently a second reactive moiety;
each instance of the first reactive moiety is capable of reacting with each instance of the second reactive moiety;
each instance of L³ is independently substituted or unsubstituted, C₁₋₂₀₀ heteroalkylene, substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkenylene, substituted or unsubstituted, C₂₋₂₀₀ heteroalkynylene, substituted or unsubstituted heterocyclylene, substituted or unsubstituted arylene, substituted or unsubstituted heteroarylene, substituted or unsubstituted carbocyclylene, or a bond, optionally wherein one or more backbone atoms of the C₁₋₂₀₀ heteroalkylene, C₁₋₂₀₀ alkylene, C₂₋₂₀₀ alkenylene, C₂₋₂₀₀ alkynylene, C₂₋₂₀₀ heteroalkenylene, or C₂₋₂₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits; and
each instance of M is independently a radical of a pharmaceutical agent or absent.

In certain embodiments, Formula B′ is Formula B:

-L¹-E²-L²-E³⁴-L³-M (B).

In another aspect, the present disclosure provides antibody-pharmaceutical agent conjugate, wherein:
n2 instances of the lysine residues of the antibody are independently modified with a moiety of Formula C′:

—(CH₂)₄-E¹²-L²-(E³⁴-L³-M)ₙ₃ (C′);
n2 is 1, 2, 3, 4, 5, or 6;
each instance of E¹² is independently —NH—C(═O)—, —NH—S(═O)—, —NH—S(═O)₂—, —NH—C(═O)—NR^{a}—, —NH—S(═O)—NR^{a}—, or —NH—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of n3 is independently 1, 2, 3, or 4;
each instance of E³⁴ is independently a moiety formed by reacting E³ with E⁴;
each instance of E³ is independently a first reactive moiety;
each instance of E⁴ is independently a second reactive moiety;
each instance of the first reactive moiety is capable of reacting with each instance of the second reactive moiety;
each instance of L³ is independently substituted or unsubstituted, C₁₋₂₀₀ heteroalkylene, substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkenylene, substituted or unsubstituted, C₂₋₂₀₀ heteroalkynylene, substituted or unsubstituted heterocyclylene, substituted or unsubstituted arylene, substituted or unsubstituted heteroarylene, substituted or unsubstituted carbocyclylene, or a bond, optionally wherein one or more backbone atoms of the C₁₋₂₀₀ heteroalkylene, C₁₋₂₀₀ alkylene, C₂₋₂₀₀ alkenylene, C₂₋₂₀₀ alkynylene, C₂₋₂₀₀ heteroalkenylene, or C₂₋₂₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits; and
each instance of M is independently a radical of a pharmaceutical agent.

In certain embodiments, the antibody-pharmaceutical agent conjugate is an antibody-pharmaceutical agent conjugate, wherein:
n2 instances of the lysine residues of the antibody are independently modified with a moiety of Formula C:

—(CH₂)₄-E¹²-L²-E³⁴-L³-M (C);
n2 is 1, 2, 3, 4, 5, or 6;
each instance of E¹² is independently —NH—C(═O)—, —NH—S(═O)—, —NH—S(═O)₂—, —NH—C(═O)—NR^{a}—, —NH—S(═O)—NR^{a}—, or —NH—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of E³⁴ is independently a moiety formed by reacting E³ with E⁴;
each instance of E³ is independently a first reactive moiety;
each instance of E⁴ is independently a second reactive moiety;
each instance of the first reactive moiety is capable of reacting with each instance of the second reactive moiety;
each instance of L³ is independently substituted or unsubstituted, C₁₋₂₀₀ heteroalkylene, substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkenylene, substituted or unsubstituted, C₂₋₂₀₀ heteroalkynylene, substituted or unsubstituted heterocyclylene, substituted or unsubstituted arylene, substituted or unsubstituted heteroarylene, substituted or unsubstituted carbocyclylene, or a bond, optionally wherein one or more backbone atoms of the C₁₋₂₀₀ heteroalkylene, C₁₋₂₀₀ alkylene, C₂₋₂₀₀ alkenylene, C₂₋₂₀₀ alkynylene, C₂₋₂₀₀ heteroalkenylene, or C₂₋₂₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits; and
each instance of M is independently a radical of a pharmaceutical agent.

In certain embodiments, Formula C′ is Formula C:

—(CH₂)₄-E¹²-L²-E³⁴-L³-M (C).

In certain embodiments, the affinity peptide has at least 80% identity to an amino acid sequence of SEQ ID NO.: 1. In certain embodiments, the affinity peptide has at least 85%, at least 90%, at least 95%, at least 98%, or at least 99% identity to the amino acid sequence of SEQ ID NO.: 1. In certain embodiments, Z33 is a peptide of an amino acid sequence of SEQ ID NO.: 1.

In certain embodiments, the affinity peptide has at least 80% identity to an amino acid sequence of SEQ ID NO.: 2, 3, 4, 5, 6, 7, 8, 9, or 10. In certain embodiments, the affinity peptide has at least 85%, at least 90%, at least 95%, at least 98%, or at least 99% identity to an amino acid sequence of SEQ ID NO.: 2, 3, 4, 5, 6, 7, 8, 9, or 10.

In certain embodiments, the affinity peptide comprises at least one cysteine or homocysteine. In certain embodiments, the affinity peptide comprises at least one cysteine or homocysteine, wherein at least one cysteine or homocysteine is modified with a moiety of Formula A′ or B′.

TABLE 15
Affinity peptide sequences.

| SEQ | |
|---|---|
| ID | |
| NO. | Sequence |
| 1 | FNMQQQRRFYEALHDPNLNEEQRNAKIKSIRDD |
| 2 | FNMQCQRRFYEALHDPNLNEEQRNAKIKSIRDDC |
| 3 | DCAWHLGELVWCT |
| 4 | RGNCAYHRGQLVWCTYH |
| 5 | KMADPNRFRGKDL |
| 6 | RGNCAYHRGKLVWCTYH |
| 7 | ADNKFNKEQQNAFYEILHLPNLNEEQRNGFIQSL |
| | KDDPSQSANLLAEAKKLNDAQXXK |
| 8 | RGNCAYHKGQLVWCTYH |
| 9 | TTYKLVINGKTLKGETTTEAVDAATAEKVFKQYA |
| | NDNGVDGEWTYDDATKTFTVTE |
| 10 | GPDCAYHRGELVWCTFH |

In certain embodiments, n1 instances of the amino acid residues of the affinity peptide are independently replaced with a moiety of Formula A. In certain embodiments, n1 instances of the amino acid residues of the affinity peptide are independently substituted with a moiety of Formula A. In certain embodiments, n1 instances of the amino acid residues of the affinity peptide are independently replaced with a moiety of Formula B. In certain embodiments, n1 instances of the amino acid residues of the affinity peptide are independently substituted with a moiety of Formula B. In certain embodiments, at least one residue that is modified (e.g., replaced or substituted) is the residue of the amino acid at the N or C terminus of the affinity peptide. In certain embodiments, at least one residue that is modified (e.g., replaced or substituted) is the residue of an amino acid at an internal position of the affinity peptide. In certain embodiments, at least one residue that is modified (e.g., replaced or substituted) is the residue of the amino acid at the 2^{nd}, 3^{rd}, 4^{th}, 5^{th}, 6^{th}, 7^{th}, 8^{th}, 9^{th}, 10^{th}, 11^{th}, 12^{th}, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, or 36^{th} position of the affinity peptide. In certain embodiments, the position is counted from the 5′ terminus, the amino acid at the 5′ terminus is at the 1^{st} position, and as the total number of the amino acids of the affinity peptide permits. In certain embodiments, at least one residue that is modified (e.g., replaced or substituted) is the residue of the amino acid at the 3^{rd} position of the affinity peptide. In certain embodiments, at least one residue that is modified (e.g., replaced or substituted) is the residue of the amino acid at the 17^{th} position of the affinity peptide. In certain embodiments, at least one residue that is modified (e.g., replaced or substituted) is the residue of the amino acid at the 20^{th} position of the affinity peptide. In certain embodiments, at least one residue that is modified (e.g., replaced or substituted) is the residue of the amino acid at the 20^{th} position of the affinity peptide of SEQ ID NO.: 1. In certain embodiments, at least one residue that is modified (e.g., replaced or substituted) is the residue of the amino acid at the 21^{st} position of the affinity peptide. In certain embodiments, at least one residue that is modified (e.g., replaced or substituted) is the residue of the amino acid at the 24^{th} position of the affinity peptide. In certain embodiments, at least one residue that is modified (e.g., replaced or substituted) is the residue of the amino acid at the 31^{st} position of the affinity peptide.

In certain embodiments, n1 is 1. In certain embodiments, n1 is 2. In certain embodiments, n1 is 3.

In certain embodiments, at least one instance of L¹ is substituted or unsubstituted, C₁₋₁₈ heteroalkylene, optionally wherein one, two, or three backbone atoms of the C₁₋₁₈ heteroalkylene are independently replaced with substituted or unsubstituted arylene, substituted or unsubstituted carbocyclylene, substituted or unsubstituted heterocyclylene, or substituted or unsubstituted heteroarylene, as valency permits. In certain embodiments, at least one instance of L¹ is substituted or unsubstituted, C₁₋₆ heteroalkylene, optionally wherein one, two, or three backbone atoms of the C₁₋₆ heteroalkylene are independently replaced with substituted or unsubstituted arylene, substituted or unsubstituted carbocyclylene, substituted or unsubstituted heterocyclylene, or substituted or unsubstituted heteroarylene, as valency permits. In certain embodiments, at least one instance of L¹ is substituted or unsubstituted, C₇₋₁₂ heteroalkylene, optionally wherein one, two, or three backbone atoms of the C₇₋₁₂ heteroalkylene are independently replaced with substituted or unsubstituted arylene, substituted or unsubstituted carbocyclylene, substituted or unsubstituted heterocyclylene, or substituted or unsubstituted heteroarylene, as valency permits. In certain embodiments, at least one instance of L¹ is substituted or unsubstituted, C₁₃₋₁₈ heteroalkylene, optionally wherein one, two, or three backbone atoms of the C₁₃₋₁₈ heteroalkylene are independently replaced with substituted or unsubstituted arylene, substituted or unsubstituted carbocyclylene, substituted or unsubstituted heterocyclylene, or substituted or unsubstituted heteroarylene, as valency permits.

The first modified affinity peptide or second modified affinity peptide, wherein at least one instance of L¹ is substituted or unsubstituted, C₁₋₁₈ heteroalkylene, optionally wherein one, two, or three backbone atoms of the C₁₋₁₈ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. The first modified affinity peptide or second modified affinity peptide, wherein at least one instance of L¹ is substituted or unsubstituted, C₁₋₆ heteroalkylene, optionally wherein one, two, or three backbone atoms of the C₁₋₆ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. The first modified affinity peptide or second modified affinity peptide, wherein at least one instance of L¹ is substituted or unsubstituted, C₇-12 heteroalkylene, optionally wherein one, two, or three backbone atoms of the C₇-12 heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. The first modified affinity peptide or second modified affinity peptide, wherein at least one instance of L¹ is substituted or unsubstituted, C₁₃₋₁₈ heteroalkylene, optionally wherein one, two, or three backbone atoms of the C₁₃₋₁₈ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits.

In certain embodiments, at least one instance of L¹ is substituted or unsubstituted, C₁₋₁₀ heteroalkylene, wherein one or two backbone atoms of the C₁₋₁₀ heteroalkylene are independently replaced with substituted or unsubstituted phenylene. In certain embodiments, at least one instance of L¹ is substituted or unsubstituted, C₁₋₃ heteroalkylene, wherein one or two backbone atoms of the C₁₋₃ heteroalkylene are independently replaced with substituted or unsubstituted phenylene. In certain embodiments, at least one instance of L¹ is substituted or unsubstituted, C₄₋₇ heteroalkylene, wherein one or two backbone atoms of the C₄₋₇ heteroalkylene are independently replaced with substituted or unsubstituted phenylene. In certain embodiments, at least one instance of L¹ is substituted or unsubstituted, C₈₋₁₀ heteroalkylene, wherein one or two backbone atoms of the C₈₋₁₀ heteroalkylene are independently replaced with substituted or unsubstituted phenylene.

In certain embodiments, at least one instance of L¹ is -(substituted or unsubstituted, C₁₋₆ heteroalkylene)₀₋₁-(substituted or unsubstituted arylene or substituted or unsubstituted heteroarylene)-(substituted or unsubstituted, C₁₋₆ heteroalkylene)₀₋₁-(substituted or unsubstituted arylene or substituted or unsubstituted heteroarylene)₀₋₁-. In certain embodiments, at least one instance of L¹ is -(substituted or unsubstituted, C₁₋₆ heteroalkylene)₀₋₁-(substituted or unsubstituted arylene or substituted or unsubstituted heteroarylene)-(substituted or unsubstituted, C₁₋₆ heteroalkylene)₀₋₁-(substituted or unsubstituted arylene or substituted or unsubstituted heteroarylene)₀₋₁-*, wherein bond * is attached to E². In certain embodiments, at least one instance of L¹ is -(substituted or unsubstituted arylene or substituted or unsubstituted heteroarylene)-. In certain embodiments, at least one instance of L¹ is -(substituted or unsubstituted, C₁₋₆ heteroalkylene)-(substituted or unsubstituted arylene or substituted or unsubstituted heteroarylene)-*, wherein bond * is attached to E². In certain embodiments, at least one instance of L¹ is -(substituted or unsubstituted, C₁₋₆ heteroalkylene)-(substituted or unsubstituted arylene or substituted or unsubstituted heteroarylene)-(substituted or unsubstituted, C₁₋₆ heteroalkylene)-(substituted or unsubstituted arylene or substituted or unsubstituted heteroarylene)-*, wherein bond * is attached to E².

In certain embodiments, at least one instance of L¹ is -(substituted or unsubstituted, C₁₋₆ heteroalkylene)₀₋₁-(substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene)-(substituted or unsubstituted, C₁₋₆ heteroalkylene)₀₋₁-(substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene)₀₋₁-*, wherein bond * is attached to E². In certain embodiments, at least one instance of L¹ is -(substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene)-. In certain embodiments, at least one instance of L¹ is -(substituted or unsubstituted, C₁₋₆ heteroalkylene)-(substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene)₀₋₁-*, wherein bond * is attached to E². In certain embodiments, at least one instance of L¹ is -(substituted or unsubstituted, C₁₋₆ heteroalkylene)-(substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene)-(substituted or unsubstituted, C₁₋₆ heteroalkylene)-(substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene)-*, wherein bond * is attached to E².

In certain embodiments, each atom in the backbone (e.g., shortest backbone) of at least one instance of L¹ is independently C, O, or S.

In certain embodiments, each substituent in at least one instance of L¹ is independently F; unsubstituted C₁₋₆ alkyl; C₁₋₆ alkyl substituted with one or more F; —O-(unsubstituted C₁₋₆ alkyl); —O—(C₁₋₆ alkyl substituted with one or more F); or oxo.

In certain embodiments, the shortest backbone length of at least one instance of L¹ is between 1 and 3, between 3 and 5, between 5 and 7, between 7 and 10, between 10 and 12, between 12 and 15, between 15 and 20, between 20 and 25, or between 25 and 30, inclusive, atoms. In certain embodiments, the shortest backbone length of at least one instance of L¹ is between 5 and 15, inclusive, atoms. In certain embodiments, the shortest backbone length of at least one instance of L¹ is between 7 and 12, inclusive, atoms.

In certain embodiments, at least one instance of L¹ is

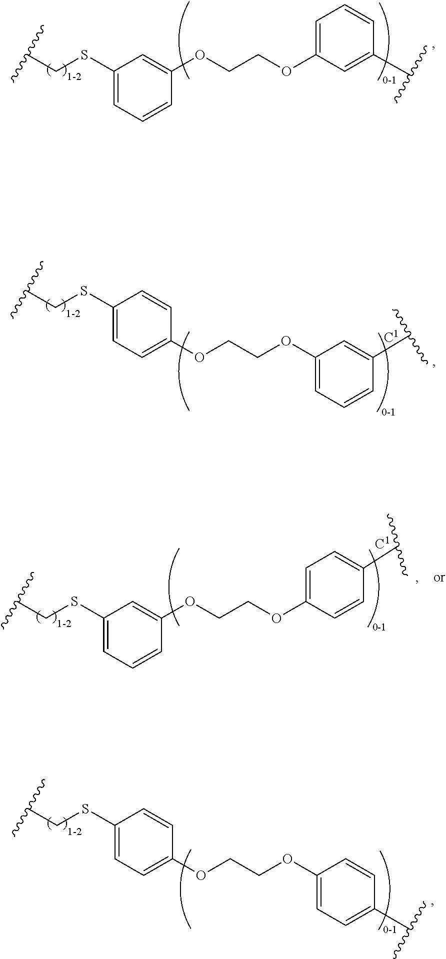

wherein bond C¹ is attached to E².

In certain embodiments, at least one instance of L¹ is

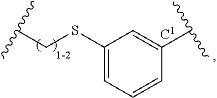

wherein bond C¹ is attached to E².

In certain embodiments, at least one instance of E² is —O—C(═O)—. In certain embodiments, at least one instance of E² is —O—C(═O)—NR^{a}—. In certain embodiments, at least one instance of E² is —O—C(═O)—NH—. In certain embodiments, at least one instance of E² is —O—S(═O)—NR^{a}— or —O—S(═O)₂—NR^{a}—. In certain embodiments, at least one instance of E² is —O—S(═O)—NH— or —O—S(═O)₂—NH—.

In certain embodiments, at least one instance of R^{a} is hydrogen. In certain embodiments, at least one instance of R^{a} is substituted or unsubstituted, C₁₋₆ alkyl. In certain embodiments, at least one instance of R^{a} is —CH₃. In certain embodiments, at least one instance of R^{a} is —C₂H₅, n-C₃H₇, or i-C₃H₇. In certain embodiments, at least one instance of R^{a} is C₁₋₆ alkyl substituted with one or more F. In certain embodiments, at least one instance of R^{a} is —CF₃. In certain embodiments, at least one instance of R^{a} is a nitrogen protecting group.

In certain embodiments, n2 is 1. In certain embodiments, n2 is 2. In certain embodiments, n2 is 3. In certain embodiments, n2 is 4, 5, or 6.

In certain embodiments, at least one instance of E¹² is —NH—C(═O)—. In certain embodiments, at least one instance of E¹² is —NH—C(═O)—NR^{a}—. In certain embodiments, at least one instance of E¹² is —NH—C(═O)—NH—. In certain embodiments, at least one instance of E¹² is —NH—S(═O)—NR^{a}— or —NH—S(═O)₂—NR^{a}—. In certain embodiments, at least one instance of E¹² is —NH—S(═O)—NH— or —NH—S(═O)₂—NH—.

In certain embodiments, the antibody is an immunoglobulin G (IgG). In certain embodiments, the antibody is an IgG1, IgG2, IgG3, or IgG4. In certain embodiments, the antibody is an immunoglobulin A (IgA), immunoglobulin D (IgD), immunoglobulin E (IgE), or immunoglobulin M (IgM). In certain embodiments, the antibody is IgA1 antibody or IgA2. In certain embodiments, the antibody is an anti-HER2 antibody (e.g., trastuzumab). In certain embodiments, the antibody is an anti-MUC1 antibody. In certain embodiments, the antibody is an anti-oncoprotein antibody, and the oncoprotein is an oncoprotein of the cancer described herein. In certain embodiments, the antibody is a monoclonal antibody. In certain embodiments, the antibody is a polyclonal antibody. In certain embodiments, the antibody is a humanized antibody. In certain embodiments, the antibody is a peptide or protein. In certain embodiments, the antibody is a peptide or protein and comprises between 10 and 30, between 30 and 100, between 100 and 300, between 300 and 1,000, between 1,000 and 3,000, or between 3,000 and 10,000, inclusive, amino acids.

In certain embodiments, n2 instances of the lysine residues of the antibody are independently substituted with a moiety of Formula C. In certain embodiments, n2 instances of the lysine residues of the antibody are independently replaced with a moiety of Formula C. In certain embodiments, at least one instance of the lysine residues that are modified (e.g., substituted or replaced) is at the heavy chain of the antibody. In certain embodiments, at least one instance of the lysine residues that are modified (e.g., substituted or replaced) is at a surface domain of the antibody. In certain embodiments, the antibody is an unmodified antibody.

In certain embodiments, at least one instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits.

In certain embodiments, at least one instance of L² is independently substituted or unsubstituted, C₁₋₁₂ alkylene, substituted or unsubstituted, C₂₋₁₂ alkenylene, substituted or unsubstituted, C₂₋₁₂ alkynylene, substituted or unsubstituted, C₁₋₁₂ heteroalkylene, substituted or unsubstituted, C₂₋₁₂ heteroalkenylene, substituted or unsubstituted, C₂₋₁₂ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₂ alkylene, C₂₋₁₂ alkenylene, C₂₋₁₂ alkynylene, C₁₋₁₂ heteroalkylene, C₂₋₁₂ heteroalkenylene, or C₂₋₁₂ heteroalkynylene are independently replaced with substituted or unsubstituted heterocyclylene, substituted or unsubstituted arylene, substituted or unsubstituted heteroarylene, or substituted or unsubstituted carbocyclylene, as valency permits.

In certain embodiments, at least one instance of L² is independently substituted or unsubstituted, C₁₂₋₄₀ alkylene, substituted or unsubstituted, C₁₂₋₄₀ alkenylene, substituted or unsubstituted, C₁₂₋₄₀ alkynylene, substituted or unsubstituted, C₁₂₋₄₀ heteroalkylene, substituted or unsubstituted, C₁₂₋₄₀ heteroalkenylene, substituted or unsubstituted, C₁₂₋₄₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₂₋₄₀ alkylene, C₁₂₋₄₀ alkenylene, C₁₂₋₄₀ alkynylene, C₁₂₋₄₀ heteroalkylene, C₁₂₋₄₀ heteroalkenylene, or C₁₂₋₄₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits.

In certain embodiments, at least one instance of L² is independently substituted or unsubstituted, C₄₀₋₁₀₀ alkylene, substituted or unsubstituted, C₄₀₋₁₀₀ alkenylene, substituted or unsubstituted, C₄₀₋₁₀₀ alkynylene, substituted or unsubstituted, C₄₀₋₁₀₀ heteroalkylene, substituted or unsubstituted, C₄₀₋₁₀₀ heteroalkenylene, substituted or unsubstituted, C₄₀₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₄₀₋₁₀₀ alkylene, C₄₀₋₁₀₀ alkenylene, C₄₀₋₁₀₀ alkynylene, C₄₀₋₁₀₀ heteroalkylene, C₄₀₋₁₀₀ heteroalkenylene, or C₄₀₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits.

In certain embodiments, at least one instance of L² is substituted or unsubstituted, C₁₋₁₀₀ alkylene or substituted or unsubstituted, C₁₋₁₀₀ heteroalkylene, optionally wherein one, two, or three backbone atoms of the C₁₋₁₀₀ alkylene or C₁₋₁₀₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L² is substituted or unsubstituted, C₁₋₁₀₀ alkylene or substituted or unsubstituted, C₁₋₁₀₀ heteroalkylene. In certain embodiments, at least one instance of L² is substituted or unsubstituted, C₁₋₁₂ alkylene or substituted or unsubstituted, C₁₋₁₂ heteroalkylene. In certain embodiments, at least one instance of L² is substituted or unsubstituted, C₁₂₋₄₀ alkylene or substituted or unsubstituted, C₁₂₋₄₀ heteroalkylene. In certain embodiments, at least one instance of L² is substituted or unsubstituted, C₄₀₋₁₀₀ alkylene or substituted or unsubstituted, C₄₀₋₁₀₀ heteroalkylene.

In certain embodiments, each atom in the backbone (e.g., shortest backbone) of at least one instance of L² is independently C, O, S, or N. In certain embodiments, each atom in the backbone (e.g., shortest backbone) of at least one instance of L² is independently C, O, or S.

In certain embodiments, each substituent in at least one instance of L² is independently F; unsubstituted C₁₋₆ alkyl; C₁₋₆ alkyl substituted with one or more F; —O— (unsubstituted C₁₋₆ alkyl); —O—(C₁₋₆ alkyl substituted with one or more F); or oxo.

In certain embodiments, the shortest backbone length of at least one instance of L² is between 1 and 3, between 3 and 5, between 5 and 7, between 7 and 10, between 10 and 12, between 12 and 15, between 15 and 20, between 20 and 25, or between 25 and 30, inclusive, atoms. In certain embodiments, the shortest backbone length of at least one instance of L² is between 2 and 10, inclusive, atoms. In certain embodiments, the shortest backbone length of at least one instance of L² is between 2 and 8, inclusive, atoms. In certain embodiments, the shortest backbone length of at least one instance of L² is between 3 and 6, inclusive, atoms.

In certain embodiments, at least one instance of L² is —(CH₂)₂₋₈—. In certain embodiments, at least one instance of L² is —(CH₂)₃₋₆—.

In certain embodiments, at least one instance of L² is substituted or unsubstituted, C₂₋₂₀ heteroalkylene, optionally wherein one or two backbone atoms of the C₂₋₂₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L² is substituted or unsubstituted, C₂₀₋₅₀ heteroalkylene, optionally wherein one, two, or three backbone atoms of the C₂₀₋₅₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L² is substituted or unsubstituted, C₅₀₋₁₀₀ heteroalkylene, optionally wherein one, two, three, or four backbone atoms of the C₅₀₋₁₀₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L² is substituted or unsubstituted, C₂₋₂₀ heteroalkylene, optionally wherein one or two backbone atoms of the C₂₋₂₀ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. In certain embodiments, at least one instance of L² is substituted or unsubstituted, C₂₀₋₅₀ heteroalkylene, optionally wherein one, two, or three backbone atoms of the C₂₀₋₅₀ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. In certain embodiments, at least one instance of L² is substituted or unsubstituted, C₅₀₋₁₀₀ heteroalkylene, optionally wherein one, two, three, or four backbone atoms of the C₅₀₋₁₀₀ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits.

In certain embodiments, at least one instance of L² is

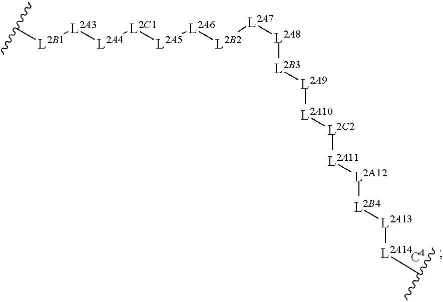

each of -L^{2A3}-L^{2A4}-, -L^{2A5}-L^{2A6}-, -L^{2A7}-L^{2A8}-, -L^{2A9}-L^{2A10}-, -L^{2A11}-L^{2A12}-, and -L^{2A13}-L^{2A14}- is independently a single bond, —O—, —S—, —S—S—, —NR^{b}—, —C(═O)O—, —C(═NR^{b})O—, —S(═O)O—, —S(═O)₂O—, —C(═O)NR^{b}—, —C(═NR^{bb})NR^{b}—, —S(═O)NR^{b}—, —S(═O)₂NR^{b}—, —OC(═O)—, —OC(═NR^{b})—, —OS(═O)—, —OS(═O)₂—, —NR^{b}C(═O)—, —NR^{b}C(═NR^{b})—, —NR^{b}S(═O)—, —NR^{b}S(═O)₂—, —OC(═O)O—, —OC(═NR^{bb})O—, —OS(═O)O—, —OS(═O)₂O—, —NR^{b}C(═O)O—, —NR^{b}C(═NR^{b})O—, —NR^{b}S(═O)O—, —NR^{b}S(═O)₂O—, —OC(═O)NR^{b}—, —OC(═NR^{b})NR^{b}—, —OS(═O)NR^{b}—, —OS(═O)₂NR^{b}—, —NR^{b}C(═O)NR^{b}—, —NR^{b}C(═NR^{b})NR^{b}—, —NR^{b}S(═O)NR^{b}—, —NR^{b}S(═O)₂NR^{b}—, —C(═O)—, —C(═NR^{b})—, —S(═O)—, —S(═O)₂—, —OP(═O)(OR^{b})O—, —SP(═O)(OR^{b})O—, —OP(═O)(OR^{b})S—, or —OP(═O)(SR^{b})O—;
each instance of R^{b} is independently hydrogen, substituted or unsubstituted, C₁₋₆ alkyl, a nitrogen protecting group when attached to a nitrogen atom, an oxygen protecting group when attached to an oxygen atom, or a sulfur protecting group when attached to a sulfur atom, or two instances of R^{b} attached to a nitrogen atom are joined with the nitrogen atom to form substituted or unsubstituted heterocyclyl or substituted or unsubstituted heteroaryl;
each of L^{2B1}, L^{2B2}, L^{2B3}, and L^{2B4} is independently a single bond, substituted or unsubstituted, C₁₋₁₀₀ alkylene, or substituted or unsubstituted, C₁₋₁₀₀ heteroalkylene;
each of L^{2C1} and L^{2C2} is independently a single bond, substituted or unsubstituted heterocyclylene or substituted or unsubstituted heteroarylene; and
bond C⁴ is attached to E³ or E³⁴.
In certain embodiments, at least one instance of L² is

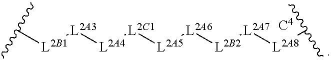

In certain embodiments, at least one instance of L² is

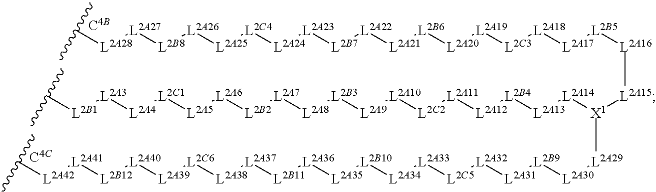

X¹ is CH or N;
each of -L^{2A3}-L^{2A4}-, -L^{2A5}-L^{2A6}-, -L^{2A7}-L^{2A8}-, -L^{2A9}-L^{2A10}-, -L^{2A11}-L^{2A12}-, -L^{2A13}-L^{2A14}-, -L^{2A15}-L^{2A16}-, -L^{2A17}-L^{2A18}-, -L^{2A19}-L^{2A20}-, -L^{2A21}-L^{2A22}-, -L^{2A23}-L^{2A24}-, -L^{2A25}-L^{2A26}-, -L^{2A27}-L^{2A28}-, -L^{2A29}-L^{2A30}-, -L^{2A31}-L^{2A32}-, -L^{2A33}-L^{2A34}-, -L^{2A35}-L^{2A36}-, -L^{2A37}-L^{2A38}-, -L^{2A39}-L^{2A40}-, and -L^{2A41}-L^{2A42}- is independently a single bond, —O—, —S—, —S—S—, —NR^{b}—, —C(═O)O—, —C(═NR^{b})O—, —S(═O)O—, —S(═O)₂O—, —C(═O)NR^{b}—, —C(═NR^{b})NR^{b}—, —S(═O)NR^{b}—, —S(═O)₂NR^{b}—, —OC(═O)—, —OC(═NR^{b})—, —OS(═O)—, —OS(═O)₂—, —NR^{b}C(═O)—, —NR^{b}C(═NR^{b})—, —NR^{b}S(═O)—, —NR^{b}S(═O)₂—, —OC(═O)O—, —OC(═NR^{b})O—, —OS(═O)O—, —OS(═O)₂O—, —NR^{b}C(═O)O—, —NR^{b}C(═NR^{b})O—, —NR^{b}S(═O)O—, —NR^{b}S(═O)₂O—, —OC(═O)NR^{b}—, —OC(═NR^{b})NR^{b}—, —OS(═O)NR^{b}—, —OS(═O)₂NR^{b}—, —NR^{b}C(═O)NR^{b}—, —NR^{b}C(═NR^{b})NR^{b}—, —NR^{b}S(═O)NR^{b}—, —NR^{b}S(═O)₂NR^{b}—, —C(═O)—, —C(═NR^{b})—, —S(═O)—, —S(═O)₂—, —OP(═O)(OR^{b})O—, —SP(═O)(OR^{b})O—, —OP(═O)(OR^{b})S—, or —OP(═O)(SR^{b})O—;
each instance of R^{b} is independently hydrogen, substituted or unsubstituted, C₁₋₆ alkyl, a nitrogen protecting group when attached to a nitrogen atom, an oxygen protecting group when attached to an oxygen atom, or a sulfur protecting group when attached to a sulfur atom, or two instances of R^{b} attached to a nitrogen atom are joined with the nitrogen atom to form substituted or unsubstituted heterocyclyl or substituted or unsubstituted heteroaryl;
each of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is independently a single bond, substituted or unsubstituted, C₁₋₉₉ alkylene, or substituted or unsubstituted, C₁₋₉₉ heteroalkylene;
each of L^{2C1}, L^{2C2}, L^{2C3}, L^{2C4}, L^{2C5}, and L^{2C6} is a single bond, substituted or unsubstituted heterocyclylene, or substituted or unsubstituted heteroarylene;
bond C^{4B} is attached to a first instance of: E³ or E³⁴, and
bond C^{4C} is attached to a second instance of: E³ or E³⁴.

In certain embodiments, at least one instance of L² is

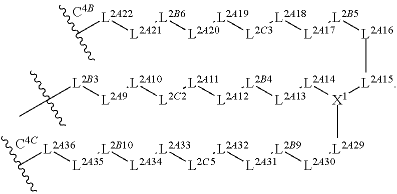

In certain embodiments, at least one instance of

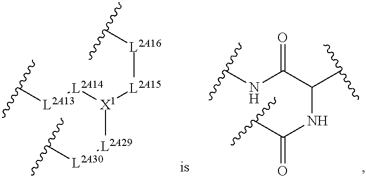

which is attached in any direction. In certain embodiments, at least one instance of

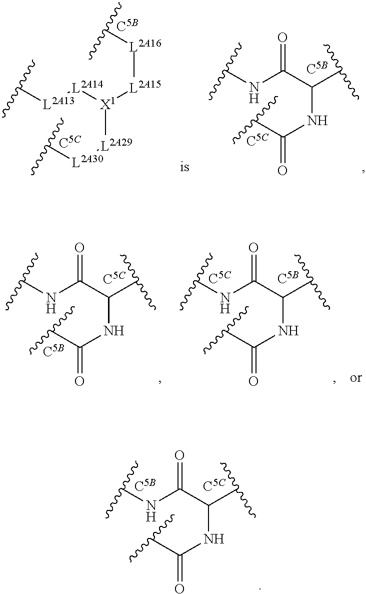

In certain embodiments, at least one of -L^{2A3}-L^{2A4}-, -L^{2A5}-L^{2A6}-, -L^{2A7}-L^{2A8}-, -L^{2A9}-L^{2A10}-, -L^{2A11}-L^{2A12}-, -L^{2A13}-L^{2A14}-, -L^{2A15}-L^{2A16}-, -L^{2A17}-L^{2A18}-, -L^{2A19}-L^{2A20}-, -L^{2A21}-L^{2A22}-, -L^{2A23}-L^{2A24}-, -L^{2A25}-L^{2A26}-, -L^{2A27}-L^{2A28}-, -L^{2A29}-L^{2A30}-, -L^{2A31}-L^{2A32}-, -L^{2A33}-L^{2A34}-, -L^{2A35}-L^{2A36}-, -L^{2A37}-L^{2A38}-, -L^{L2A39}-L^{2A40}-, and -L^{2A41}-L^{2A42}- is a single bond, —O—, —C(═O)NR^{b}—, —NR^{b}C(═O)—, —NR^{b}C(═O)O—, —OC(═O)NR^{b}—, —NR^{b}C(═O)NR^{b}—, or —C(═O)—. In certain embodiments, at least one of -L^{2A3}-L^{2A4}-, -L^{2A5}-L^{2A6}-, -L^{2A7}-L^{2A8}-, -L^{2A9}-L^{2A10}-, -L^{2A11}-L^{2A12}-, -L^{2A13}-L^{2A14}-, -L^{2A15}-L^{2A16}-, -L^{2A17}-L^{2A18}-, -L^{2A19}-L^{2A20}-, -L^{2A21}-L^{2A22}-, -L^{2A23}-L^{2A24}-, -L^{2A25}-L^{2A26}-, -L^{2A27}-L^{2A28}-, -L^{2A29}-L^{2A30}-, -L^{2A31}-L^{2A32}-, -L^{2A33}-L^{2A34}-, -L^{2A35}-L^{2A36}-, -L^{2A37}-L^{2A38}-, -L^{2A39}-L^{2A40}-, and -L^{2A41}-L^{2A42}- is a single bond, —O—, —C(═O)NR^{b}—, —NR^{b}C(═O)—, or —C(═O)—. In certain embodiments, at least one of -L^{2A3}-L^{2A4}-, -L^{2A5}-L^{2A6}-, -L^{2A7}-L^{2A8}-, -L^{2A9}-L^{2A10}-, -L^{2A11}-L^{2A12}-, -L^{2A13}-L^{2A14}-, -L^{2A15}-L^{2A16}-, -L^{2A17}-L^{2A18}-, -L^{2A19}-L^{2A20}-, -L^{2A21}-L^{2A22}-, -L^{2A23}-L^{2A24}-, -L^{2A25}-L^{2A26}-, -L^{2A27}-L^{2A28}-, -L^{2A29}-L^{2A30}-, -L^{2A31}-L^{2A32}-, -L^{2A33}-L^{2A34}-, -L^{2A35}-L^{2A36}-, -L^{2A37}-L^{2A38}-, -L^{2A39}-L^{2A40}-, and -L^{2A41}-L^{2A42}- is a single bond, —O—, —C(═O)NH—, —NHC(═O)—, —NHC(═O)O—, —OC(═O)NH—, —NHC(═O)NH—, or —C(═O)—. In certain embodiments, at least one of -L^{2A3}-L^{2A4}-, -L^{2A5}-L^{2A6}-, -L^{2A7}-L^{2A8}-, -L^{2A9}-L^{2A10}-, -L^{2A11}-L^{2A12}, -L^{2A13}-L^{2A14}-, -L^{2A15}-L^{2A16}-, -L^{2A17}-L^{2A18}-, -L^{2A19}-L^{2A20}-, -L^{2A21}-L^{2A22}-, -L^{2A23}-L^{2A24}-, -L^{2A25}-L^{2A26}-, -L^{2A27}-L^{2A28}-, -L^{2A29}-L^{2A30}-, -L^{2A31}-L^{2A32}-, -L^{2A33}-L^{2A34}-, -L^{2A35}-L^{2A36}-, -L^{L2A37}-L^{2A38}-, -L^{2A39}-L^{2A40}- and -L^{2A41}-L^{2A42}- is a single bond, —O—, —C(═O)NH—, —NHC(═O)—, or —C(═O)—.

In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is a single bond, unsubstituted C₁₋₁₀₀ alkylene, —(OCH₂CH₂)₁₋₅₀—, —(CH₂CH₂O)₁₋₅₀—, or —(CH₂OCH₂)₁₋₅₀—.

In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is unsubstituted C₁₋₁₂ alkylene. In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is unsubstituted C₁₂₋₄₀ alkylene. In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is unsubstituted C₁₂₋₄₀ alkylene. In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is unsubstituted C₄₀₋₉₉ alkylene. In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is —(OCH₂CH₂)₁₋₆—, —(CH₂CH₂O)₁₋₆—, or —(CH₂OCH₂)₁₋₆—. In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is —(OCH₂CH₂)₆₋₂₀—, —(CH₂CH₂O)₆₋₂₀—, or —(CH₂OCH₂)₆₋₂₀—. In certain embodiments, at least one instance of L^{2B1}, L^{2B2}, L^{2B3}, L^{2B4}, L^{2B5}, L^{2B6}, L^{2B7}, L^{2B8}, L^{2B9}, L^{2B10}, L^{2B11}, and L^{2B12} is —(OCH₂CH₂)₂₀₋₄₉—, —(CH₂CH₂O)₂₀₋₄₉—, or —(CH₂OCH₂)₂₀₋₄₉—.

In certain embodiments, at least one instance of L^{2C1} and L^{2C2} is a single bond,

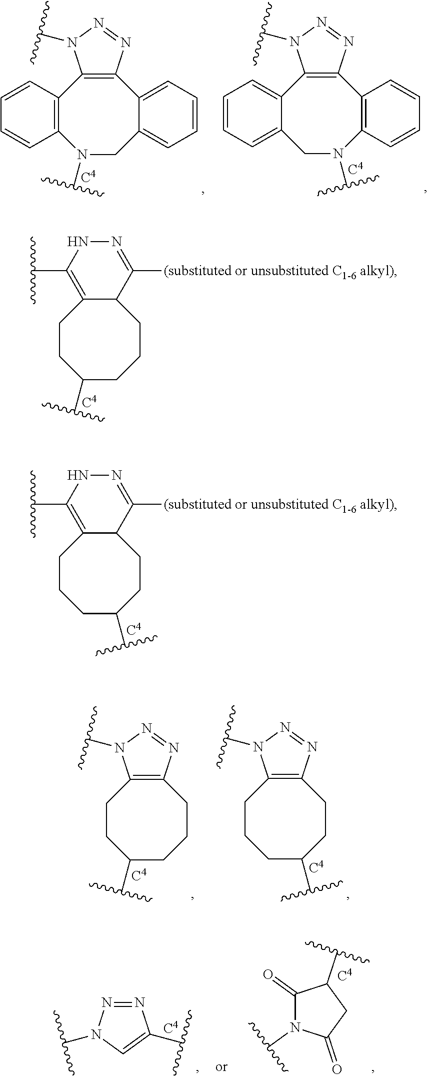

wherein bond C⁴ is attached to L^{2A4}, L^{2A5}, L^{2A10}, or L^{2A11}.

In certain embodiments, at least one instance of n3 is 1. In certain embodiments, at least one instance of n3 is 2. In certain embodiments, at least one instance of n3 is 3 or 4. When n3 is 2, 3, or 4, any 2 instances of E³ may be attached to the same atom (e.g., N or C) or different atoms of L² as valency permits.

In certain embodiments, at least one instance of E³ is an electrophile. In certain embodiments, at least one instance of E³ is a first click-chemistry handle. Any “click chemistry” reaction known in the art can be used to this end. Click chemistry is a chemical approach introduced by Sharpless in 2001 and describes chemistry tailored to generate substances quickly and reliably by joining small units together. See, e.g., Kolb, Finn and Sharpless Angewandte Chemie International Edition (2001) 40: 2004-2021; Evans, Australian Journal of Chemistry (2007) 60: 384-395). Exemplary coupling reactions (some of which may be classified as “click chemistry”) include formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition; thiol-yne addition; imine formation; Michael additions (e.g., maleimide addition); and Diels-Alder reactions (e.g., tetrazine [4+2] cycloaddition). Examples of click chemistry reactions can be found in, e.g., Kolb, H. C.; Finn, M. G. and Sharpless, K. B. Angew. Chem. Int. Ed. 2001, 40, 2004-2021. Kolb, H. C. and Shrapless, K. B. Drug Disc. Today, 2003, 8, 112-1137; Rostovtsev, V. V.; Green L. G.; Fokin, V. V. and Shrapless, K. B. Angew. Chem. Int. Ed. 2002, 41, 2596-2599; Tomoe, C. W.; Christensen, C. and Meldal, M. J. Org. Chem. 2002, 67, 3057-3064. Wang, Q. et al. J. Am. Chem. Soc. 2003, 125, 3192-3193; Lee, L. V. et al. J. Am. Chem. Soc. 2003 125, 9588-9589; Lewis, W. G. et al. Angew. Chem. Int. Ed. 2002, 41, 1053-41057; Manetsch, R. et al., J. Am. Chem. Soc. 2004, 126, 12809-12818; Mocharla, V. P. et al. Angew. Chem., Int. Ed. 2005, 44, 116-120. In certain embodiments, at least one instance of E³ is —N₃ or substituted or unsubstituted 1,2,4,5-tetrazinyl. In certain embodiments, at least one instance of E³ is —N₃. In certain embodiments, at least one instance of E³ is

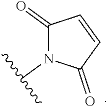 [SMILES: *N1C(=O)C=CC1=O]

In certain embodiments, at least one instance of E³ is —C(═O)-(a leaving group), —S(═O)-(a leaving group), —S(═O)₂-(a leaving group), or —P(═O)(R^{c})-(a leaving group), wherein R^{c} is substituted or unsubstituted, C₁₋₆ alkyl, substituted or unsubstituted phenyl, —O-(substituted or unsubstituted, C₁₋₆ alkyl), or —O-(substituted or unsubstituted phenyl).

In certain embodiments, at least one instance of E³ comprises non-aromatic, C≡C or C≡C. In certain embodiments, at least one instance of E³ is —C≡CH, substituted or unsubstituted cyclooctynyl optionally fused independently with one or more instances of substituted or unsubstituted phenyl, substituted or unsubstituted cyclopropenyl, substituted or unsubstituted cyclobutenyl, substituted or unsubstituted trans-cyclooctenyl optionally fused independently with one or more instances of substituted or unsubstituted phenyl, or substituted or unsubstituted

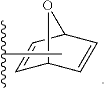

In certain embodiments, at least one instance of E³ is substituted or unsubstituted

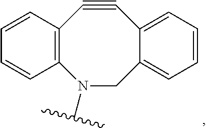 [SMILES: *N1Cc2ccccc2C#Cc2ccccc21]

substituted or unsubstituted

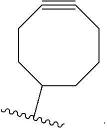 [SMILES: *C1CCC#CCCC1]

substituted or unsubstituted trans-cyclooctenyl, or —CH≡CH. In certain embodiments, at least one instance of E³ is

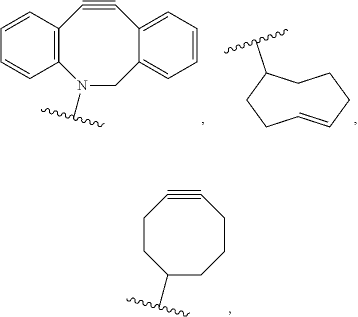 [SMILES: *N1Cc2ccccc2C#Cc2ccccc21 ; *C1CC/C=C/CCC1 ; *C1CCC#CCCC1]

### or —CH≡CH.

In certain embodiments, at least one instance of E³ is —SH.

In certain embodiments, at least one instance of E³ is a nucleophile. In certain embodiments, at least one instance of E⁴ is a second click-chemistry handle. In certain embodiments, the second click-chemistry handle is orthogonal to the first click-chemistry handle (e.g., the second click-chemistry handle is capable of undergoing a click-chemistry reaction with the first click-chemistry handle (e.g., under suitable (e.g., ambient or physiological) conditions). In certain embodiments, at least one instance of E⁴ comprises non-aromatic, C≡C or C═C. In certain embodiments, at least one instance of E⁴ is —C≡CH, substituted or unsubstituted cyclooctynyl optionally fused independently with one or more instances of substituted or unsubstituted phenyl, substituted or unsubstituted cyclopropenyl, substituted or unsubstituted cyclobutenyl, substituted or unsubstituted trans-cyclooctenyl optionally fused independently with one or more instances of substituted or unsubstituted phenyl, or substituted or unsubstituted

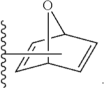

In certain embodiments, at least one instance of E⁴ is substituted or unsubstituted

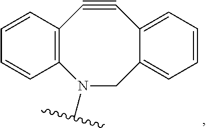 [SMILES: *N1Cc2ccccc2C#Cc2ccccc21]

substituted or unsubstituted

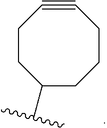 [SMILES: *C1CCC#CCCC1]

substituted or unsubstituted trans-cyclooctenyl, or —CH≡CH. In certain embodiments, at least one instance of E⁴ is

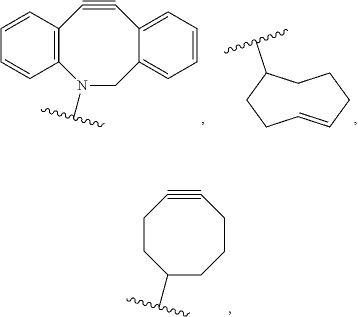 [SMILES: *N1Cc2ccccc2C#Cc2ccccc21 ; *C1CC/C=C/CCC1 ; *C1CCC#CCCC1]

or —CH≡CH. In certain embodiments, at least one instance of E⁴ is —SH. In certain embodiments, at least one instance of E⁴ is —OH, —NH₂, or —NH-(substituted or unsubstituted, C₁₋₆ alkyl).

In certain embodiments, at least one instance of E⁴ is —N₃ or substituted or unsubstituted 1,2,4,5-tetrazinyl. In certain embodiments, at least one instance of E⁴ is —N₃. In certain embodiments, at least one instance of E⁴ is

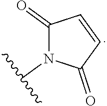 [SMILES: *N1C(=O)C=CC1=O]

In certain embodiments, at least one instance of E³ is —N₃ or substituted or unsubstituted 1,2,4,5-tetrazinyl, and at least one instance of E⁴ is —C≡CH, substituted or unsubstituted cyclooctynyl optionally fused independently with one or more instances of substituted or unsubstituted phenyl, substituted or unsubstituted cyclopropenyl, substituted or unsubstituted cyclobutenyl, substituted or unsubstituted trans-cyclooctenyl optionally fused independently with one or more instances of substituted or unsubstituted phenyl, or substituted or unsubstituted

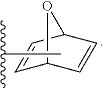

In certain embodiments, at least one instance of E³ is —N₃ or substituted or unsubstituted 1,2,4,5-tetrazinyl, and at least one instance of E⁴ is substituted or unsubstituted

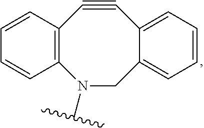 [SMILES: *N1Cc2ccccc2C#Cc2ccccc21]

substituted or unsubstituted

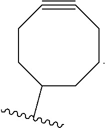 [SMILES: *C1CCC#CCCC1]

substituted or unsubstituted trans-cyclooctenyl, or —CH≡CH. In certain embodiments, at least one instance of E³ is

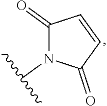 [SMILES: *N1C(=O)C=CC1=O]

and at least one instance of E⁴ is —SH.

In certain embodiments, at least one instance of E⁴ is —N₃ or substituted or unsubstituted 1,2,4,5-tetrazinyl, and at least one instance of E³ is —C≡CH, substituted or unsubstituted cyclooctynyl optionally fused independently with one or more instances of substituted or unsubstituted phenyl, substituted or unsubstituted cyclopropenyl, substituted or unsubstituted cyclobutenyl, substituted or unsubstituted trans-cyclooctenyl optionally fused independently with one or more instances of substituted or unsubstituted phenyl, or substituted or unsubstituted

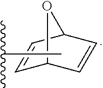

In certain embodiments, at least one instance of E⁴ is —N₃ or substituted or unsubstituted 1,2,4,5-tetrazinyl, and at least one instance of E³ is substituted or unsubstituted

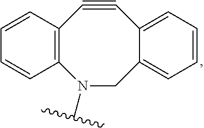 [SMILES: *N1Cc2ccccc2C#Cc2ccccc21]

substituted or unsubstituted

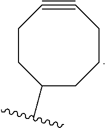 [SMILES: *C1CCC#CCCC1]

substituted or unsubstituted trans-cyclooctenyl, or —CH≡CH. In certain embodiments, at least one instance of E⁴ is

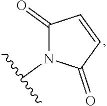 [SMILES: *N1C(=O)C=CC1=O]

and at least one instance of E³ is —SH.

When n3 is 2, 3, or 4, any 2 instances of E³⁴ may be attached to the same atom (e.g., N or C) or different atoms of L² as valency permits.

In certain embodiments, at least one instance of E³⁴ is

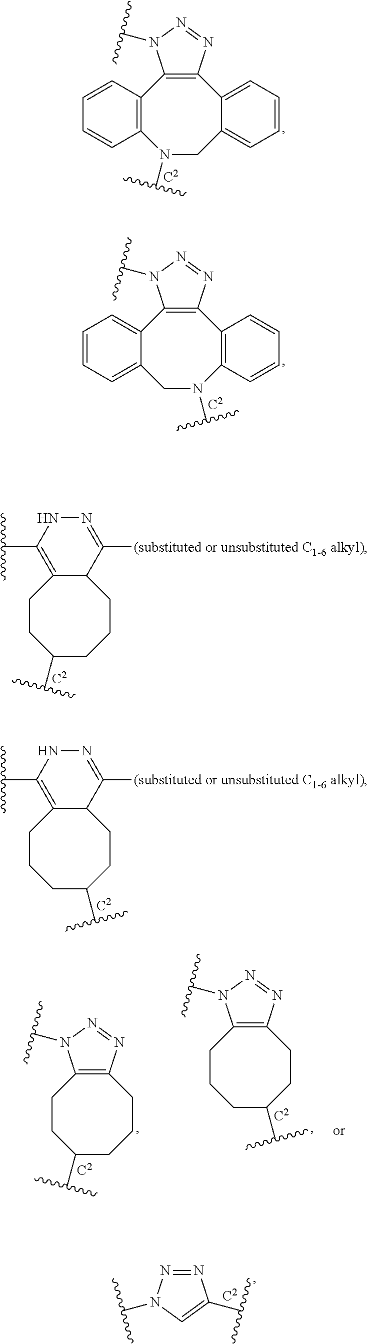

wherein bond C² is attached to L² or L³. In certain embodiments, at least one instance of E³⁴ is

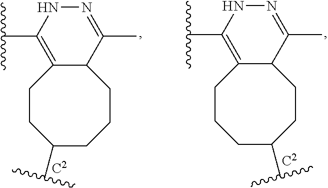 [SMILES: *C1=C2CCC(*)CCCC2C(C)=NN1 ; *C1=C2CCCC(*)CCC2C(C)=NN1]

wherein bond C² is attached to L² or L³. In certain embodiments, at least one instance of E³⁴ is

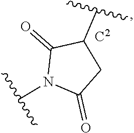 [SMILES: *C1CC(=O)N(*)C1=O]

wherein bond C² is attached to L³ or L². In certain embodiments, bond C² is attached to L³.

In certain embodiments, at least one instance of L³ is

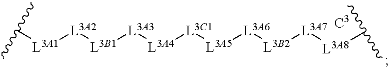

each of -L^{3A1}-L^{3A2}-, -L^{3A3}-L^{3A4}-, -L^{3A5}-L^{3A6}-, and -L^{3A7}-L^{3A8}- is independently a single bond, —O—, —S—, —S—S—, —NR^{b}—, —C(═O)O—, —C(═NR^{b})O—, —S(═O)O—, —S(═O)₂O—, —C(═O)NR^{b}—, —C(═NR^{b})NR^{b}—, —S(═O)NR^{b}—, —S(═O)₂NR^{b}—, —OC(═O)—, —OC(═NR^{b})—, —OS(═O)—, —OS(═O)₂—, —NR^{b}C(═O)—, —NR^{b}C(═NR^{b})—, —NR^{b}S(═O)—, —NR^{b}S(═O)₂—, —OC(═O)O—, —OC(═NR^{b})O—, —OS(═O)O—, —OS(═O)₂O—, —NR^{b}C(═O)O—, —NR^{b}C(═NR^{b})O—, —NR^{b}S(═O)O—, —NR^{b}S(═O)₂O—, —OC(═O)NR^{b}—, —OC(═NR^{b})NR^{b}—, —OS(═O)NR^{b}—, —OS(═O)₂NR^{b}—, —NR^{b}C(═O)NR^{b}—, —NR^{b}C(═NR^{b})NR^{b}—, —NR^{b}S(═O)NR^{b}—, —NR^{b}S(═O)₂NR^{b}—, —C(═O)—, —C(═NR^{b})—, —S(═O)—, —S(═O)₂—, —OP(═O)(OR^{b})O—, —SP(═O)(OR^{b})O—, —OP(═O)(OR^{b})S—, or —OP(═O)(SR^{b})O—;
each instance of R^{b} is independently hydrogen, substituted or unsubstituted, C₁₋₆ alkyl, a nitrogen protecting group when attached to a nitrogen atom, an oxygen protecting group when attached to an oxygen atom, or a sulfur protecting group when attached to a sulfur atom, or two instances of R^{b} attached to a nitrogen atom are joined with the nitrogen atom to form substituted or unsubstituted heterocyclyl or substituted or unsubstituted heteroaryl;
each of L^{3B1} and L^{3B2} is independently a single bond, substituted or unsubstituted, C₁₋₁₀₀ alkylene, or substituted or unsubstituted, C₁₋₁₀₀ heteroalkylene;
L^{3C1} is a single bond, substituted or unsubstituted heterocyclylene or substituted or unsubstituted heteroarylene; and
bond C³ is attached to M.

In certain embodiments, at least one instance of L³ is

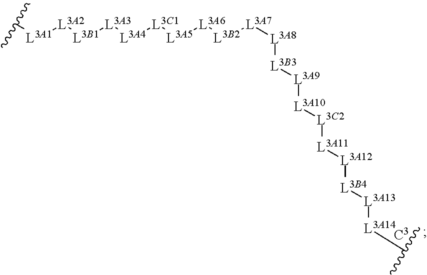

each of -L^{3A1}-L^{3A2}-, -L^{3A3}-L^{3A4}-, -L^{3A5}-L^{3A6}-, -L^{3A7}-L^{3A8}-, -L^{3A9}-L^{3A10}-, -L^{3A11}-L^{3A12}-, and -L^{3A13}-L^{3A14}- is independently a single bond, —O—, —S—, —S—S—, —NR^{b}—, —C(═O)O—, —C(═NR^{b})O—, —S(═O)O—, —S(═O)₂O—, —C(═O)NR^{b}—, —C(═NR^{b})NR^{b}—, —S(═O)NR^{b}—, —S(═O)₂NR^{b}—, —OC(═O)—, —OC(═NR^{b})—, —OS(═O)—, —OS(═O)₂—, —NR^{b}C(═O)—, —NR^{b}C(═NR^{b})—, —NR^{b}S(═O)—, —NR^{b}S(═O)₂—, —OC(═O)O—, —OC(═NR^{b})O—, —OS(═O)O—, —OS(═O)₂O—, —NR^{b}C(═O)O—, —NR^{b}C(═NR^{b})O—, —NR^{b}S(═O)O—, —NR^{b}S(═O)₂O—, —OC(═O)NR^{b}—, —OC(═NR^{b})NR^{b}—, —OS(═O)NR^{b}—, —OS(═O)₂NR^{b}—, —NR^{b}C(═O)NR^{b}—, —NR^{b}C(═NR^{b})NR^{b}—, —NR^{b}S(═O)NR^{b}—, —NR^{b}S(═O)₂NR^{b}—, —C(═O)—, —C(═NR^{b})—, —S(═O)—, —S(═O)₂—, —OP(═O)(OR^{b})O—, —SP(═O)(OR^{b})O—, —OP(═O)(OR^{b})S—, or —OP(═O)(SR^{b})O—;
each instance of R^{b} is independently hydrogen, substituted or unsubstituted, C₁₋₆ alkyl, a nitrogen protecting group when attached to a nitrogen atom, an oxygen protecting group when attached to an oxygen atom, or a sulfur protecting group when attached to a sulfur atom, or two instances of R^{b} attached to a nitrogen atom are joined with the nitrogen atom to form substituted or unsubstituted heterocyclyl or substituted or unsubstituted heteroaryl;
each of L^{3B1}, L^{3B2}, L^{3B3}, and L^{3B4} is independently a single bond, substituted or unsubstituted, C₁₋₁₀₀ alkylene, or substituted or unsubstituted, C₁₋₁₀₀ heteroalkylene;
each of L^{3C1} and L^{3C2} is independently a single bond, substituted or unsubstituted heterocyclylene or substituted or unsubstituted heteroarylene; and
bond C³ is attached to M.

In certain embodiments, at least one of -L^{3A1}-L^{3A2}-, -L^{3A3}-L^{3A4}-, -L^{3A5}-L^{3A6}-, -L^{3A7}-L^{3A8}-, -L^{3A9}-L^{3A10}-, -L^{3A11}-L^{3A12}-, and -L^{3A13}-L^{3A14}- is a single bond, —O—, —C(═O)NR^{b}—, —NR^{b}C(═O)—, —NR^{b}C(═O)O—, —OC(═O)NR^{b}—, —NR^{b}C(═O)NR^{b}—, or —C(═O)—. In certain embodiments, at least one of -L^{3A1}-L^{3A2}-, -L^{3A3}-L^{3A4}-, -L^{3A5}-L^{3A6}-, -L^{3A7}-L^{3A8}-, -L^{3A9}-L^{3A10}-, -L^{3A11}-L^{3A12}-, and -L^{3A13}-L^{3A14}- is a single bond, —O—, —C(═O)NR^{b}—, —NR^{b}C(═O)—, or —C(═O)—. In certain embodiments, at least one of -L^{3A1}-L^{3A2}-, -L^{3A3}-L^{3A4}-, -L^{3A5}-L^{3A6}-, -L^{3A7}-L^{3A8}-, -L^{3A9}-L^{3A10}-, -L^{3A11}-L^{3A12}- and -L^{3A13}-L^{3A14}- is a single bond, —O—, —C(═O)NH—, —NHC(═O)—, —NHC(═O)O—, —OC(═O)NH—, —NHC(═O)NH—, or —C(═O)—. In certain embodiments, at least one of -L^{3A1}-L^{3A2}-, -L^{3A3}-L^{3A4}-, -L^{3A5}-L^{3A6}-, -L^{3A7}-L^{3A8}-, -L^{3A9}-L^{3A10}-, -L^{3A11}-L^{3A12}-, and -L^{3A13}-L^{3A14}- is a single bond, —O—, —C(═O)NH—, —NHC(═O)—, or —C(═O)—. In certain embodiments, each of -L^{3A1}-L^{3A2}-, -L^{3A3}-L^{3A4}-, -L^{3A5}-L^{3A6}-, -L^{3A7}-L^{3A8}-, -L^{3A9}-L^{3A10}-, -L^{3A11}-L^{3A12}- and -L^{3A13}-L^{3A14}- is independently a single bond, —O—, —NR^{b}—, —C(═O)NR^{b}—, or —NR^{b}C(═O)—. In some embodiments, each of -L^{3A1}-L^{3A2}-, -L^{3A3}-L^{3A4}-, -L^{3A5}-L^{3A6}-, -L^{3A7}-L^{3A8}-, -L^{3A9}-L^{3A10}-, -L^{3A11}-L^{3A12}-, and -L^{3A13}-L^{3A14}- is independently a single bond, —O—, —NH—, —C(═O)NH—, or —NHC(═O)—.

In certain embodiments, at least one instance of R^{b} is hydrogen. In certain embodiments, at least one instance of R^{b} is substituted or unsubstituted, C₁₋₆ alkyl. In certain embodiments, at least one instance of R^{b} is —CH₃. In certain embodiments, at least one instance of R^{b} is —C₂H₅, n-C₃H₇, or i-C₃H₇. In certain embodiments, at least one instance of R^{b} is C₁₋₆ alkyl substituted with one or more F. In certain embodiments, at least one instance of R^{b} is —CF₃. In certain embodiments, at least one instance of R^{b} is a nitrogen protecting group.

In certain embodiments, each of L^{3B1}, L^{3B2}, L^{3B3}, and L^{3B4} is independently a single bond, substituted or unsubstituted, C₁₋₂₀ alkylene or substituted or unsubstituted, C₁₋₂₀ heteroalkylene. In certain embodiments, each of L^{3B1}, L^{3B2}, L^{3B3}, and L^{3B4} is independently substituted or unsubstituted, C₁₋₁₀ alkylene or substituted or unsubstituted, C₁₋₁₀ heteroalkylene. In certain embodiments, each L^{3B1}, L^{3B2}, L^{3B3}, and L^{3B4} is independently unsubstituted C₁₋₁₀ alkylene. In certain embodiments, each of L^{3B1}, L^{3B2}, L^{3B3}, and L^{3B4} independently consists of one, two, three, four, five, six, seven, eight, nine, or ten PEG repeats. In certain embodiments, at least one of L^{3B1}, L^{3B2}, L^{3B3}, and L^{3B4} is a single bond, unsubstituted C₁₋₂₀₀ alkylene, —(OCH₂CH₂)₁₋₁₀₀—, —(CH₂CH₂O)₁₋₁₀₀—, or —(CH₂OCH₂)₁₋₁₀₀—. In certain embodiments, at least one of L^{3B1}, L^{3B2}, L^{3B3}, and L^{3B4} is unsubstituted C₁₋₁₂ alkylene. In certain embodiments, at least one of L^{3B1}, L^{3B2}, L^{3B3}, and L^{3B4} is unsubstituted C₁₂₋₄₀ alkylene. In certain embodiments, at least one of L^{3B1}, L^{3B2}, L^{3B3}, and L^{3B4} is unsubstituted C₄₀₋₁₀₀ alkylene. In certain embodiments, at least one of L^{3B1}, L^{3B2}, L^{3B3}, and L^{3B4} is unsubstituted C₁₀₀₋₂₀₀ alkylene. In certain embodiments, at least one of L^{3B1}, L^{3B2}, L^{3B3}, and L^{3B4} is —(OCH₂CH₂)₁₋₆—, —(CH₂CH₂O)₁₋₆—, or —(CH₂OCH₂)₁₋₆—. In certain embodiments, at least one of L^{3B1}, L^{3B2}, L^{3B3}, and L^{3B4} is —(OCH₂CH₂)₆₋₂₀—, —(CH₂CH₂O)₆₋₂₀—, or —(CH₂OCH₂)₆₋₂₀—. In certain embodiments, at least one of L^{3B1}, L^{3B2}, L^{3B3}, and L^{3B4} is —(OCH₂CH₂)₂₀₋₅₀—, —(CH₂CH₂O)₂₀₋₅₀—, or —(CH₂OCH₂)₂₀₋₅₀—. In certain embodiments, at least one of L^{3B1}, L^{3B2}, L^{3B3}, and L^{3B4} is —(OCH₂CH₂)₅₀₋₁₀₀—, —(CH₂CH₂O)₅₀₋₁₀₀—, or —(CH₂OCH₂)₅₀₋₁₀₀—.

In certain embodiments, at least one instance of L^{3C1} and L^{3C2} is a single bond,

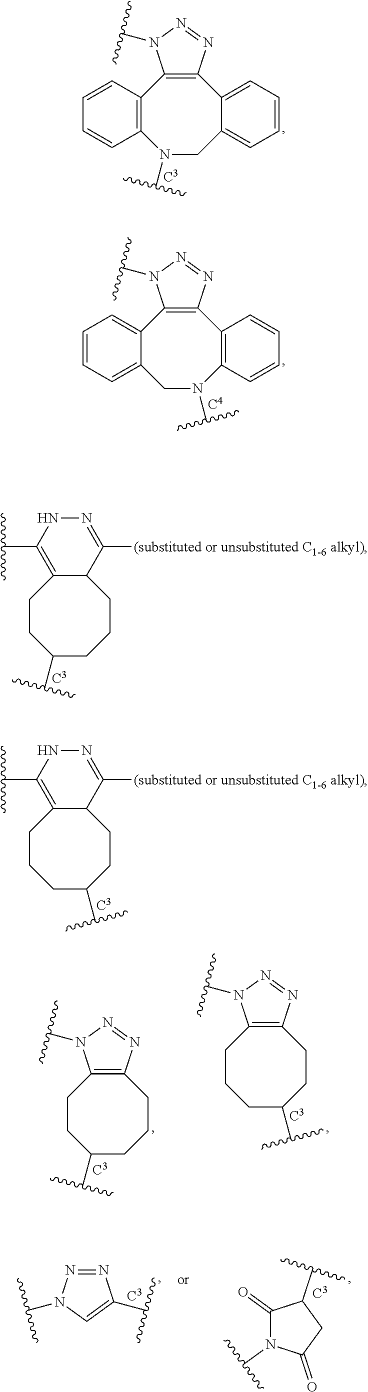

wherein bond C³ is attached to L^{3A4}, L^{3A5}, L^{3A10}, or L^{3A11}.

In certain embodiments, at least one instance of L³ is substituted or unsubstituted, C₁₂₋₂₀₀ alkylene or substituted or unsubstituted, C₁₂₋₂₀₀ heteroalkylene, optionally wherein one, two, or three backbone atoms of the C₁₂₋₂₀₀ alkylene or C₁₂₋₂₀₀ heteroalkylene are 16 independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L³ is substituted or unsubstituted, C₂₋₂₀ heteroalkylene, optionally wherein one or two backbone atoms of the C₂₋₂₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L³ is substituted or unsubstituted, C₂₀₋₅₀ heteroalkylene, optionally wherein one, two, or three backbone atoms of the C₂₀₋₅₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L³ is substituted or unsubstituted, C₅₀₋₁₀₀ heteroalkylene, optionally wherein one, two, three, or four backbone atoms of the C₅₀₋₁₀₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L³ is substituted or unsubstituted, C₁₀₀₋₂₀₀ heteroalkylene, optionally wherein one, two, three, four, or five backbone atoms of the C₁₀₀₋₂₀₀ heteroalkylene are independently replaced with substituted or unsubstituted heterocyclylene, substituted or unsubstituted arylene, substituted or unsubstituted heteroarylene, or substituted or unsubstituted carbocyclylene, as valency permits. In certain embodiments, at least one instance of L³ is substituted or unsubstituted, C₂₋₂₀ heteroalkylene, optionally wherein one or two backbone atoms of the C₂₋₂₀ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. In certain embodiments, at least one instance of L³ is substituted or unsubstituted, C₂₀₋₅₀ heteroalkylene, optionally wherein one, two, or three backbone atoms of the C₂₀₋₅₀ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. In certain embodiments, at least one instance of L³ is substituted or unsubstituted, C₅₀₋₁₀₀ heteroalkylene, optionally wherein one, two, three, or four backbone atoms of the C₅₀₋₁₀₀ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits. In certain embodiments, at least one instance of L³ is substituted or unsubstituted, C₁₀₀₋₂₀₀ heteroalkylene, optionally wherein one, two, three, four, or five backbone atoms of the C₁₀₀₋₂₀₀ heteroalkylene are independently replaced with substituted or unsubstituted phenylene, substituted or unsubstituted naphthylene, substituted or unsubstituted, monocyclic, 5- or 6-membered heteroarylene, or substituted or unsubstituted, bicyclic, 8- to 10-membered heteroarylene, as valency permits.

In certain embodiments, at least one instance of L³ is substituted or unsubstituted, C₁₋₁₂ heteroalkylene. In certain embodiments, at least one instance of L³ is substituted or unsubstituted, C₁₂₋₄₀ heteroalkylene. In certain embodiments, at least one instance of L³ is substituted or unsubstituted, C₄₀₋₁₀₀ heteroalkylene. In certain embodiments, at least one instance of L³ is substituted or unsubstituted, C₁₀₀₋₁₇₀ heteroalkylene. In certain embodiments, at least one instance of L³ is substituted or unsubstituted, C₁₇₀₋₂₀₀ heteroalkylene. In certain embodiments, at least one instance of L³ is substituted or unsubstituted, C₄₀₋₁₇₀ heteroalkylene.

The second modified affinity peptide or antibody-pharmaceutical agent conjugate, wherein at least one instance of L³ is —CH₂—, —O—, —NH—, —N(CH₃)—, —CH₂CH₂O—, —OCH₂CH₂—, —C(═O)—, —C(═O)NH—, —C(═O)N(CH₃)—, —NHC(═O)—, or —N(CH₃)C(═O)—, or a combination thereof, provided that:
the number of backbone atoms of the combination is between 10 and 100, inclusive; and
the combination does not comprise O—O, O—N, N—O, N—N, or C(═O)—C(═O). In certain embodiments, the combination described in this paragraph includes all permutations, e.g., —CH₂—CH₂—, —CH₂—O—, —O—CH₂—O—, and —O—CH₂—CH₂—O—.

In certain embodiments, each atom in the backbone (e.g., shortest backbone) of at least one instance of L³ is independently C, O, S, or N. In certain embodiments, each atom in the backbone (e.g., shortest backbone) of at least one instance of L³ is independently C, O, or N.

In certain embodiments, each substituent in at least one instance of L³ is independently F; unsubstituted C₁₋₆ alkyl; C₁₋₆ alkyl substituted with one or more F; —O— (unsubstituted C₁₋₆ alkyl); —O—(C₁₋₆ alkyl substituted with one or more F); or oxo.

In certain embodiments, the shortest backbone length of at least one instance of L³ is between 1 and 6, between 6 and 12, between 12 and 40, between 40 and 70, between 70 and 100, between 100 and 130, between 130 and 170, or between 170 and 200, inclusive, atoms. In certain embodiments, the shortest backbone length of at least one instance of L³ is between 40 and 170, inclusive, atoms. In certain embodiments, the shortest backbone length of at least one instance of L³ is between 50 and 150, inclusive, atoms. In certain embodiments, the shortest backbone length of at least one instance of L³ is between 60 and 130, inclusive, atoms.

In certain embodiments, at least one instance of M is a radical of a pharmaceutical agent. In certain embodiments, at least one instance of the pharmaceutical agent is a peptide, protein, polynucleotide, or small molecule. In certain embodiments, at least one instance of the pharmaceutical agent is a therapeutic agent, prophylactic agent, or diagnostic agent. In certain embodiments, at least one instance of the pharmaceutical agent is a therapeutic agent. In certain embodiments, at least one instance of the pharmaceutical agent is an actoprotector, Addison disease medication, alcohol deterrent, anti-aging agent, anti-hypoxic agent, anti-infective agent, anti-inflammatory agent, anti-neurodegenerative agent, antidiabetic agent, antidote, antifibrotic agent, antigout agent, antihistamine, antiobesity agent, antiosteoporotic agent, antitumor agent, antiulcer agent, antivenom, autoimmune disease medication, biopharmaceutical, COPD medication, calmidazolium, calmidazolium chloride, cardiovascular agent, Celiac disease medication, Crohn disease medication, cytoprotective agent, demulcent, dermatological agent, epignetic drug, G-quadruplex stabilizer, gastrointestinal agent, Graves disease medication, hematologic agent, hormone antagonist, lysosomotropic agent, Meniere disease medication, metabolic agent, natriuretic, nervous system agent, neuromuscular agent, ophthalmic agent, osmotherapy agent, oxytocic agent, pharmaceutical immune agent, pharmaceutical chemosensitizer, pharmaceutical photosensitizer, pharmacoenhancer, photothermal agent, pulmonary surfactant, radiomimetic, radiopharmaceutical, renal agent, reproductive control agent, respiratory system agent, secretagogue, sequestering agent, sialagogue, sodium-iodide symporter inhibitor, sonosensitizer, vilaprisan, or wound healing promoter. In certain embodiments, at least one instance of the pharmaceutical agent is an antidiabetic agent or antiobesity agent. In certain embodiments, at least one instance of the pharmaceutical agent is a glucagon-like peptide-1 receptor agonist, β-secretase 2 inhibitor, insulin mimetic, insulin secretagogue, or insulin sensitizer. In certain embodiments, at least one instance of the pharmaceutical agent is a glucagon-like peptide-1 receptor agonist. In certain embodiments, at least one instance of the pharmaceutical agent is semaglutide, tirzepatide, AC 163794, albiglutide, danuglipron, danuglipron tromethamine, dapiglutide, dulaglutide, ecnoglutide, efinopegdutide, efocipegtrutide, exenatide, liraglutide, lixisenatide, mazdutide, pemvidutide, sitagliptin, taspoglutide, utreglutide, or vurolenatide, or a pharmaceutically acceptable salt thereof. In certain embodiments, at least one instance of the pharmaceutical agent is semaglutide or tirzepatide. In certain embodiments, at least one instance of the pharmaceutical agent is liraglutide.

In certain embodiments, at least one instance of the pharmaceutical agent is an anti-cancer agent. In certain embodiments, at least one instance of the pharmaceutical agent is monomethyl auristatin E, abiraterone acetate, ABVD, ABVE, ABVE-PC, AC, AC-T, ADE, ado-trastuzumab emtansine, afatinib dimaleate, aldesleukin, alemtuzumab, anastrozole, arsenic trioxide, asparaginase Erwinia chrysanthemi, axitinib, azacitidine, BEACOPP, belinostat, bendamustine hydrochloride, BEP, bevacizumab, bicalutamide, bleomycin, blinatumomab, bortezomib, bosutinib, brentuximab vedotin, busulfan, cabazitaxel, cabozantinib-s-malate, CAF, capecitabine, CAPOX, carboplatin, carboplatin-taxol, carfilzomibcarmustine, carmustine implant, ceritinib, cetuximab, chlorambucil, chlorambucil-prednisone, CHOP, cisplatin, clofarabine, CMF, COPP, COPP-ABV, crizotinib, CVP, cyclophosphamide, cytarabine, dabrafenib, dacarbazine, dactinomycin, dasatinib, daunorubicin hydrochloride, decitabine, degarelix, denileukin diftitox, denosumab, Dinutuximab, docetaxel, doxorubicin hydrochloride, doxorubicin hydrochloride liposome, enzalutamide, epirubicin hydrochloride, EPOCH, erlotinib hydrochloride, etoposide, etoposide phosphate, everolimus, exemestane, FEC, fludarabine phosphate, fluorouracil, FOLFIRI, FOLFIRI-BEVACIZUMAB, FOLFIRI-CETUXIMAB, FOLFIRINOX, FOLFOX, FU-LV, fulvestrant, gefitinib, gemcitabine hydrochloride, gemcitabine-cisplatin, gemcitabine-oxaliplatin, goserelin acetate, Hyper-CVAD, ibritumomab tiuxetan, ibrutinib, ICE, idelalisib, ifosfamide, imatinib mesylate, imiquimod, ipilimumab, irinotecan hydrochloride, ixabepilone, lanreotide acetate, lapatinib ditosylate, lenalidomide, lenvatinib, letrozole, leucovorin calcium, leuprolide acetate, liposomal cytarabine, lomustine, mechlorethamine hydrochloride, megestrol acetate, mercaptopurine, methotrexate, mitomycin c, mitoxantrone hydrochloride, MOPP, nelarabine, nilotinib, nivolumab, obinutuzumab, OEPA, ofatumumab, OFF, olaparib, omacetaxine mepesuccinate, OPPA, oxaliplatin, paclitaxel, paclitaxel albumin-stabilized nanoparticle formulation, PAD, palbociclib, pamidronate disodium, panitumumab, panobinostat, pazopanib hydrochloride, pegaspargase, peginterferon alfa-2b, pembrolizumab, pemetrexed disodium, pertuzumab, plerixafor, pomalidomide, ponatinib hydrochloride, pralatrexate, prednisone, procarbazine hydrochloride, radium 223 dichloride, raloxifene hydrochloride, ramucirumab, R-CHOP, recombinant HPV bivalent vaccine, recombinant human papillomavirus, nonavalent vaccine, quadrivalent vaccine, recombinant interferon alfa-2b, regorafenib, rituximab, romidepsin, ruxolitinib phosphate, siltuximab, sipuleucel-t, sorafenib tosylate, STANFORD V, sunitinib malate, TAC, tamoxifen citrate, temozolomide, temsirolimus, thalidomide, thiotepa, topotecan hydrochloride, toremifene, tositumomab and iodine I 131, tositumomab, TPF, trametinib, trastuzumab, VAMP, vandetanib, VEIP, vemurafenib, vinblastine sulfate, vincristine sulfate, vincristine sulfate liposome, vinorelbine tartrate, vismodegib, vorinostat, XELIRI, XELOX, ziv-aflibercept, or zoledronic acid, or a pharmaceutically acceptable salt thereof.

In certain embodiments, at least one instance of the pharmaceutical agent is a prophylactic agent. In certain embodiments, at least one instance of the pharmaceutical agent is an antibiotic, nutritional supplement, vaccine, interleukin, interferon, or cytokine. Vaccines may comprise isolated proteins or peptides, inactivated organisms and viruses, dead organisms and viruses, genetically altered organisms or viruses, and cell extracts.

In certain embodiments, at least one instance of the pharmaceutical agent is a diagnostic agent. In certain embodiments, at least one instance of the pharmaceutical agent is selected from the group consisting of fluorescent molecules; gases; metals; imaging agents, such as commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials. In certain embodiments, at least one instance of the pharmaceutical agent is used in magnetic resonance imaging (MRI), such as iron oxide particles or gadolinium complexes. Gadolinium complexes that have been approved for clinical use include gadolinium chelates with DTPA, DTPA-BMA, DOTA and HP-DO3A which are reviewed in Aime, et al. (Chemical Society Reviews (1998), 27:19-29).

In certain embodiments, at least one instance of the pharmaceutical agent is a metal, inorganic compound, organometallic compound, organic compound, or salt thereof. In certain embodiments, the imaging agent contains a metal selected from the group consisting of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, dubnium, seaborgium, bohrium, hassium, meitnerium, gadolinium, gallium, thallium, and barium. In certain embodiments, the imaging agent is a magnetic resonance imaging (MRI) agent. In certain embodiments, the MRI agent is gadolinium. In certain embodiments, the MRI agent is a nitroxide radical-containing compound. In certain embodiments, the imaging agent is a nuclear medicine imaging agent. In certain embodiments, the nuclear medicine imaging agent is selected from the group consisting of ⁶⁴Cu diacetyl-bis(N⁴-methylthiosemicarbazone) (⁶⁴Cu-ASTM), ¹⁸F-fluorodeoxyglucose (FDG), ¹⁸F-fluoride, 3′-deoxy-3′-[¹⁸F]fluorothymidine (FLT), ¹⁸F-fluoromisonidazole (FMISO), gallium, technetium-99m, and thallium. In certain embodiments, the imaging agent is radiographic imaging agent. In certain embodiments, the radiographic imaging agent is selected from the group consisting of barium, gastrografin, and iodine contrast agent. In certain embodiments, at least one instance of the pharmaceutical agent comprises a fluorescent molecule, a metal chelate, a contrast agent, a radionuclide, or a positron emission tomography (PET) imaging agent, an infrared imaging agent, a near-IR imaging agent, a computer assisted tomography (CAT) imaging agent, a photon emission computerized tomography imaging agent, an X-ray imaging agent, or a magnetic resonance imaging (MRI) agent. In certain embodiments, at least one instance of the pharmaceutical agent comprises a radionuclide. In some embodiments, at least one instance of the pharmaceutical agent is a fluorescent molecule. In some embodiments, the fluorescent molecule comprises an acridine dye, a cyanine dye, a rhodamine dye, a BODIPY dye, a fluorescein dye, a dansyl dye, an Alexa dye, an atto dye, a quantum dot, or a fluorescent protein. In some embodiments, the fluorescent molecule is a cyanine dye (e.g., Cy3, Cy 3.5, Cy5, Cy5.5, Cy7, or Cy7.5). In some embodiments, at least one instance of the pharmaceutical agent is an MRI agent (e.g., a contrast agent). Examples of suitable materials for use as MRI agents (e.g., contrast agents) include gadolinium chelates, as well as iron, magnesium, manganese, copper, and chromium. In some embodiments, at least one instance of the pharmaceutical agent is a CAT imaging agent or an X-ray imaging agent. Examples of materials useful for CAT and X-ray imaging include iodine-based materials. In some embodiments, at least one instance of the pharmaceutical agent is a PET imaging agent. Examples of suitable PET imaging agents include compounds and compositions comprising the positron emitting radioisotopoes ¹⁸F, ¹⁵O, ¹³N, ¹¹C, ⁸²Rb, ⁶⁴Cu, and ⁶⁸Ga, e.g., fludeoxyglucose (¹⁸F-FDG), ⁶⁸Ga-DOTA-psuedopeptides (e.g., ⁶⁸Ga-DOTA-TOC), ¹¹C-metomidate, ¹¹C-acetate, ¹¹C-methionine, ¹¹C-choline, ¹⁸F-fluciclovine, ¹⁸F-fluorocholine, ¹⁸F-fluorodeoxysorbitol, ¹⁸F-3′-fluoro-3′-deoxythymidine, ¹¹C-raclopride, and ¹⁸F-desmethoxyfallypride. In some embodiments, at least one instance of the pharmaceutical agent is a near-IR imaging agent. Examples of near-IR imaging agents include Pz 247, DyLight 750, DyLight 800, cyanine dyes (e.g., Cy5, Cy5.5, Cy7), AlexaFluor 680, AlexaFluor 750, IRDye 680, IRDye 800CW, and Kodak X-SIGHT dyes. In certain embodiments, at least one instance of the pharmaceutical agent is a contrast agent. In certain embodiments, at least one instance of the contrast agent is a magnetic-resonance signal enhancing agent, X-ray attenuating agent, ultrasound scattering agent, or ultrasound frequency shifting agent.

In some embodiments, at least one instance of the pharmaceutical agent is a radionuclide. Among the radionuclides used, gamma-emitters, positron-emitters, and X-ray emitters may be suitable for diagnostic and/or therapy, while beta emitters and alpha-emitters may also be used for therapy. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Sc, ⁹⁰Y, ⁸⁸Y, ⁹⁷Ru, ¹⁰⁰Pd, ^{101m}Rh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁷Cu, ⁷⁵Br, ⁷⁷Br, ^{99m}Tc, ¹⁴C, ¹³N, ¹⁵O, ³²P, ³³P, and ¹⁸F.

In certain embodiments, each instance of the pharmaceutical agent is a therapeutic agent. In certain embodiments, each instance of the pharmaceutical agent is a prophylactic agent. In certain embodiments, each instance of the pharmaceutical agent is a diagnostic agent. In certain embodiments, at least one instance of the pharmaceutical agent is a therapeutic agent, and at least one instance of the pharmaceutical agent is a diagnostic agent. In certain embodiments, each instance of the pharmaceutical agent is the same. In certain embodiments, at least two instances of the pharmaceutical agent are different from each other.

### Compositions, Kits, and Methods of Use

In another aspect, the present disclosure provides a composition comprising a first modified affinity peptide, second modified affinity peptide, or antibody-pharmaceutical agent conjugate; and optionally one or more excipients.

In certain embodiments, the composition comprises an effective amount of the antibody-pharmaceutical agent conjugate. In certain embodiments, the composition is a pharmaceutical composition. In certain embodiments, the excipients are pharmaceutically acceptable excipients.

In certain embodiments, the compositions are useful for delivering a pharmaceutical agent to a subject in need thereof, cell, tissue, or biological sample. In certain embodiments, the compositions are useful for treating a disease in a subject in need thereof. In certain embodiments, the compositions are useful for preventing a disease in a subject in need thereof. In certain embodiments, the compositions are useful for diagnosing a disease in a subject in need thereof.

In certain embodiments, the subject is an animal. In certain embodiments, the subject is a mammal. In certain embodiments, the subject is a human. In certain embodiments, the subject is a human two-years and older. In certain embodiments, the subject is a human eighteen-years and older.

In certain embodiments, the cell, tissue, or biological sample is in vitro. In certain embodiments, the cell is in vivo.

Pharmaceutical compositions described herein can be prepared by any method known in the art of pharmacology. In general, such preparatory methods include bringing the antibody-pharmaceutical agent conjugate (which includes a pharmaceutical agent (the “active ingredient”)) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly (vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly (vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the antibody-pharmaceutical agent conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the antibody-pharmaceutical agent conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an antibody-pharmaceutical agent conjugate described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the antibody-pharmaceutical agent conjugate in powder form through the outer layers of the skin to the dermis are suitable.

Formulations suitable for topical administration include liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops May further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The antibody-pharmaceutical agent conjugates are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The antibody-pharmaceutical agent conjugates and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the antibody-pharmaceutical agent conjugate or pharmaceutical composition described herein is suitable for topical administration to the eye of a subject.

The exact amount of an antibody-pharmaceutical agent conjugate required to achieve an effective amount will vary from subject to subject, depending, for example, on species, age, and general condition of a subject, severity of the side effects or disorder, identity of the particular antibody-pharmaceutical agent conjugate, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an antibody-pharmaceutical agent conjugate described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an antibody-pharmaceutical agent conjugate described herein. In certain embodiments, a dose described herein includes independently between 1 mg and 3 mg, inclusive, of an antibody-pharmaceutical agent conjugate described herein. In certain embodiments, a dose described herein includes independently between 3 mg and 10 mg, inclusive, of an antibody-pharmaceutical agent conjugate described herein. In certain embodiments, a dose described herein includes independently between 10 mg and 30 mg, inclusive, of an antibody-pharmaceutical agent conjugate described herein. In certain embodiments, a dose described herein includes independently between 30 mg and 100 mg, inclusive, of an antibody-pharmaceutical agent conjugate described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, the composition further comprises one or more additional pharmaceutical agents. The antibody-pharmaceutical agent conjugate or composition can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including an antibody-pharmaceutical agent conjugate described herein and an additional pharmaceutical agent shows a synergistic effect that is absent in a pharmaceutical composition including one of the antibody-pharmaceutical agent conjugate and the additional pharmaceutical agent, but not both.

The antibody-pharmaceutical agent conjugate or composition can be administered concurrently with, prior to, or subsequent to one or more additional pharmaceutical agents, which are different from the pharmaceutical agent included in the antibody-pharmaceutical agent conjugate or composition. This may be useful as, e.g., combination therapies. In certain embodiments, the additional pharmaceutical agent is a pharmaceutical agent described in the “First modified affinity peptides, second modified affinity peptides, and antibody-pharmaceutical agent conjugates” section of the present disclosure. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the antibody-pharmaceutical agent conjugate or composition described herein in a single dose or administered separately in different doses. The particular combination to employ in a regimen will take into account compatibility of the antibody-pharmaceutical agent conjugate described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, and pain-relieving agents. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is a binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the antibody-pharmaceutical agent conjugate described herein or pharmaceutical composition can be administered in combination with an anti-cancer therapy including surgery, radiation therapy, transplantation (e.g., stem cell transplantation, bone marrow transplantation), immunotherapy, and chemotherapy.

In another aspect, the present disclosure provides a kit comprising:
a first modified affinity peptide, second modified affinity peptide, antibody-pharmaceutical agent conjugate, or composition; and
instructions for using the first modified affinity peptide, second modified affinity peptide, antibody-pharmaceutical agent conjugate, or composition.

In certain embodiments, the kit comprises a first container. In certain embodiments, the first container comprises the first modified affinity peptide, second modified affinity peptide, antibody-pharmaceutical agent conjugate, or composition. In some embodiments, the kit further comprises a second container. In certain embodiments, the second container comprises the instructions. In certain embodiments, the instructions comprise information required by a regulatory agency, such as the U.S. Food and Drug Administration (FDA) or European Medicines Agency (EMA). In certain embodiments, the instructions comprise prescribing information. In certain embodiments, the second container comprises the first container. In some embodiments, the kit further comprises a third container. In certain embodiments, the third container comprises the excipients. In certain embodiments, the third container comprises the additional pharmaceutical agents. In certain embodiments, the second container comprises the third container. In certain embodiments, each of the first, second, and third containers is independently a vial, ampule, bottle, syringe, dispenser package, tube, or box.

In another aspect, the present disclosure provides a method comprising administering to a subject in need thereof an effective amount of a first modified affinity peptide, a second modified affinity peptide, an antibody-pharmaceutical agent conjugate, or a composition.

In another aspect, the present disclosure provides a method of delivering a pharmaceutical agent to a cell comprising contacting the cell with an effective amount of: the antibody-pharmaceutical agent conjugate, or a tautomer, isotopically labeled conjugate, or salt thereof; or the composition.

In another aspect, the present disclosure provides a method comprising contacting a cell, tissue, or biological sample with a first modified affinity peptide, second modified affinity peptide, antibody-pharmaceutical agent conjugate, or a composition.

In certain embodiments, the subject is in need of treatment of a disease. In certain embodiments, the subject is in need of prevention of a disease. In certain embodiments, the subject is in need of diagnosis of a disease.

In certain embodiments, the subject is in need of treatment of a disease, and at least one instance of the pharmaceutical agent is a therapeutic agent. In certain embodiments, the subject is in need of prevention of a disease, and at least one instance of the pharmaceutical agent is a prophylactic agent. In certain embodiments, the subject is in need of diagnosis of a disease, and at least one instance of the pharmaceutical agent is a diagnostic agent.

In certain embodiments, the disease is a metabolic disease, cancer, benign neoplasm, pathologic angiogenesis, inflammatory disease, autoimmune disease, hematological disease, genetic disease, neurological disease, painful condition, or psychiatric disease.

In certain embodiments, the disease is a metabolic disease. In certain embodiments, the disease is diabetes. In certain embodiments, the disease is Type I diabetes. In certain embodiments, the disease is Type II diabetes. In certain embodiments, the disease is gestational diabetes. In certain embodiments, the disease is obesity. In certain embodiments, the disease is hyperglycemia. In certain embodiments, the disease is hyperinsulinemia. In certain embodiments, the disease is insulin resistance.

In certain embodiments, the disease is cancer. In certain embodiments, the disease is a solid tumor. In certain embodiments, the disease is a hematological malignancy. In certain embodiments, the disease is leukemia. In certain embodiments, the disease is lymphoma.

In certain embodiments, the administration is parenteral administration. In certain embodiments, the administration is intramuscular, subcutaneous, or intracerebroventricular administration. In certain embodiments, the administration is intrathecal administration. In certain embodiments, the administration is intravenous administration. In certain embodiments, the administration is intracerebroventricular administration. In certain embodiments, the administration is oral administration. In certain embodiments, the administration is topical administration.

In certain embodiments, the method further comprises administering to or implanting in the subject in need thereof an effective amount of an additional therapy. In certain embodiments, the additional therapy is an additional pharmaceutical agent. In certain embodiments, the additional therapy is surgery, radiation, or transplantation.

### EXAMPLES

In order that the embodiments described herein may be more fully understood, the following examples are set forth. The examples described in this application are offered to illustrate the oligonucleotides, pharmaceutical compositions, uses, and methods provided herein and are not to be construed in any way as limiting their scope.

### Example 1. A Platform to Paint Native Antibodies with Therapeutic Payloads

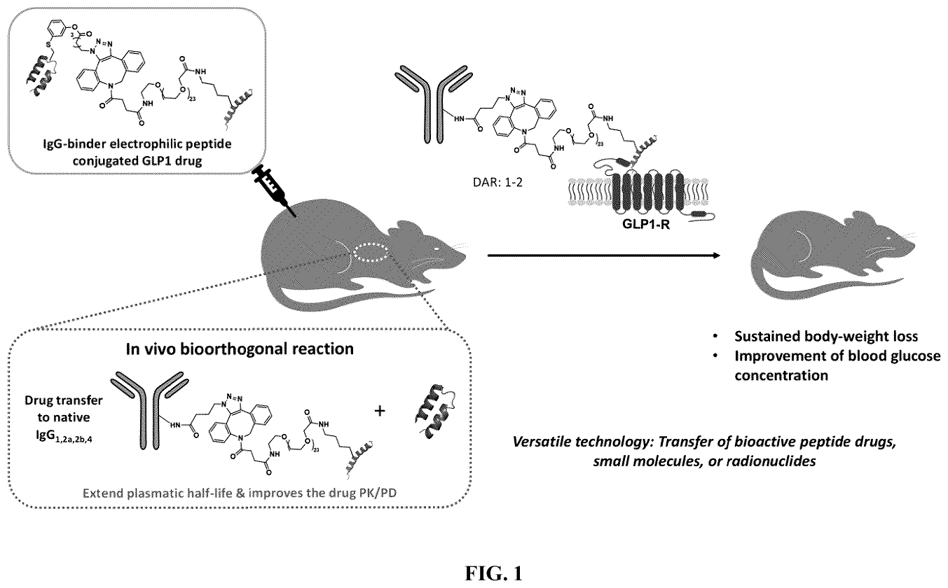

A platform to “paint” native antibodies with therapeutic payloads was developed and tested for the ability to use the circulating antibodies as a vehicle to extend the PK/PD of peptide drugs after in vivo administration in mice (FIG. 1). Briefly, in this method, the IgG-binder electrophile peptide is first injected in mice, either intravenously, subcutaneously, or intraperitoneally. Then, a selective biorthogonal transfer reaction occurs in vivo between the electrophile and the lysine K317 of the Fc fragment of native circulating IgGs. The covalent GLP1 drug transfer to IgGs results in long-acting GLP1 with enhanced pharmacokinetic (PK) and pharmacodynamic (PD) characteristics.

### In Vitro and In Vivo Azido Transfer to IgGs

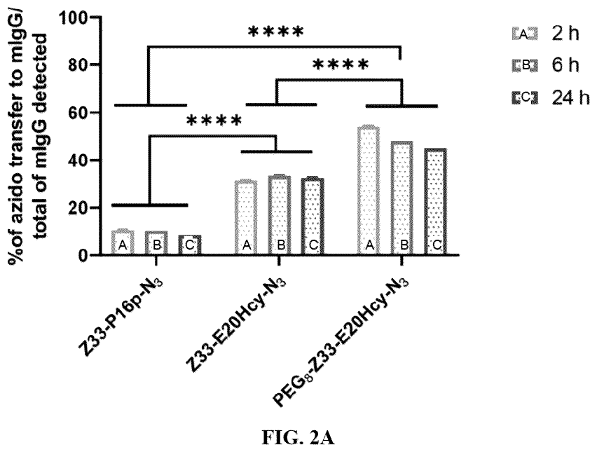

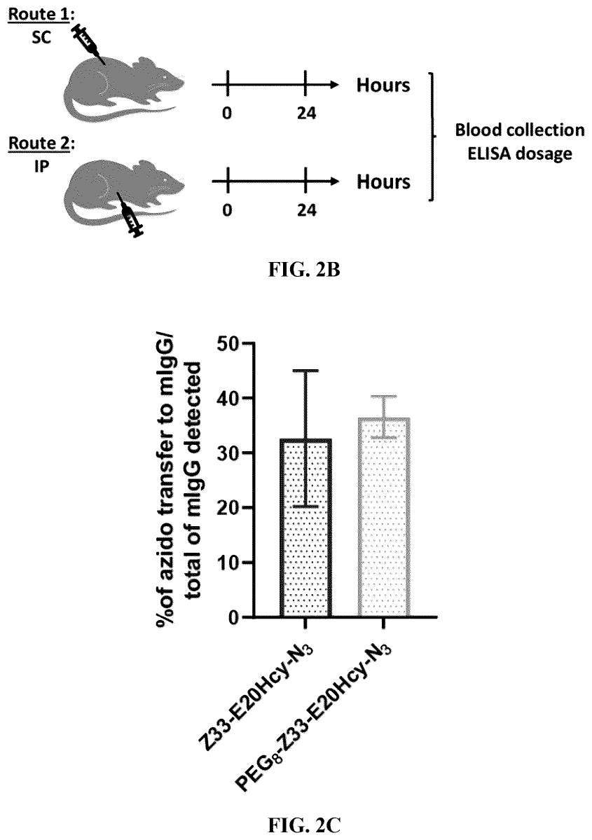

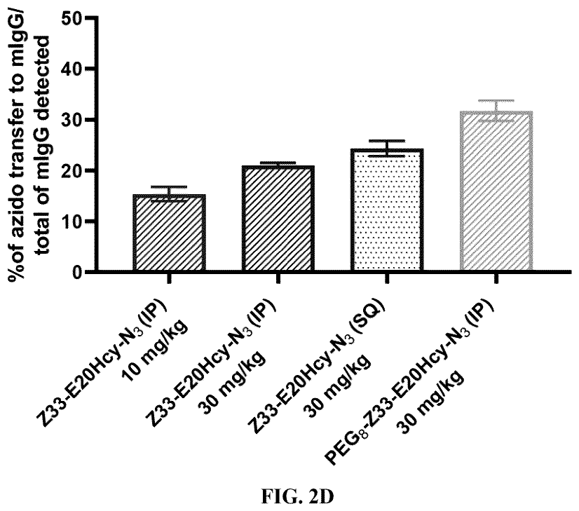

The structures of the IgG-binder peptides Z33-P16p, Z33-E20Hcy, PEG8-Z33-E20Hcy and their reactivity towards human IgG1, IgG2, and IgG3 and mouse IgG1, IgG2a, and IgG2b was first evaluated in vitro. The IgG-binder peptides were also incubated in mouse sera for 2-, 6-, and 24-hours to evaluate the percentage of in vitro azido transfer to mouse IgG (FIG. 2A). The electrophile peptides were found to possess a high affinity and selectivity towards IgG in mouse sera.

To evaluate the in vivo azido transfer in mice, two groups of mice were injected subcutaneously or intraperitoneally with the IgG-binder peptides. Mice were sacrificed at 24 hours post-injection, and the blood was collected for ELISA analysis. Sandwich ELISA assays. ELISA analysis of the azido transfer to mouse IgG following the subcutaneous injection of 30 mg/kg of Z33-E20Hcy or pegylated-Z33-E20Hcy indicated that both performed similarly (mean 30-40% azido transfer), with the pegylated-Z33-E20Hcy performing moderately better (FIG. 2C). The percentage azido transfer was also compared with intraperitoneal injection of 10 mg/kg Z33-E20Hcy, intraperitoneal injection of 30 mg/kg of Z33-E20Hcy, subcutaneous injection of 30 mg/kg Z33-E20Hcy, and intraperitoneal injection of 30 mg/kg pegylated-Z33-E20Hcy (FIG. 2D).

### IgG Painting Using GLP1 Drugs for Improving Drug PK/PD

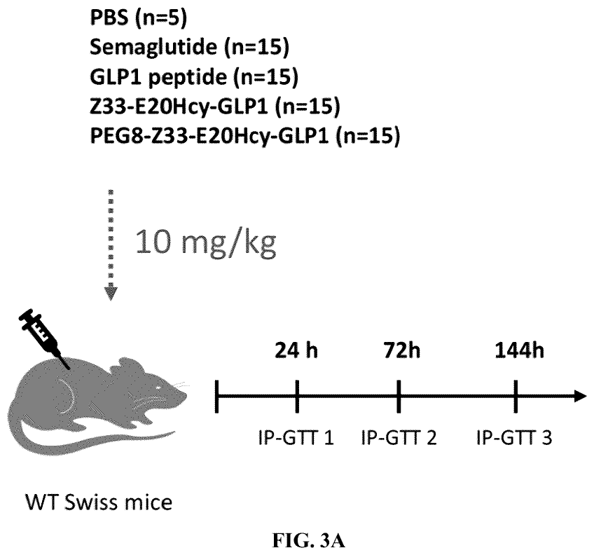

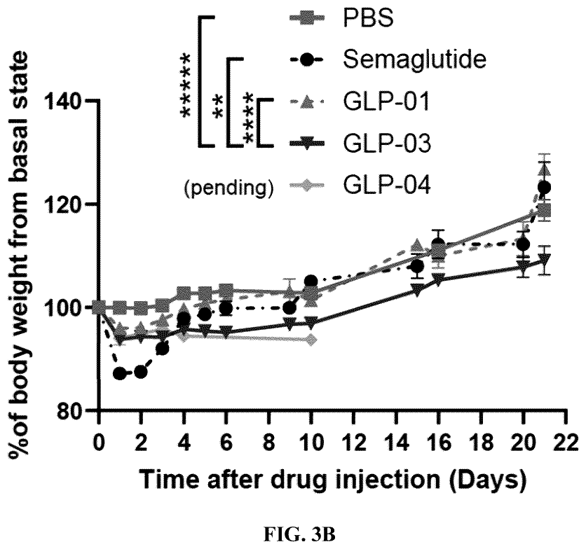

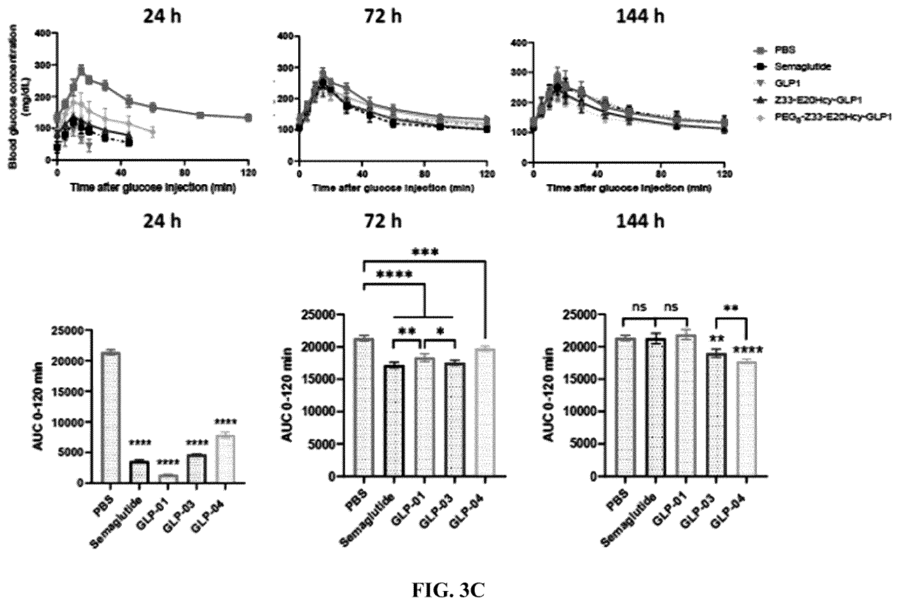

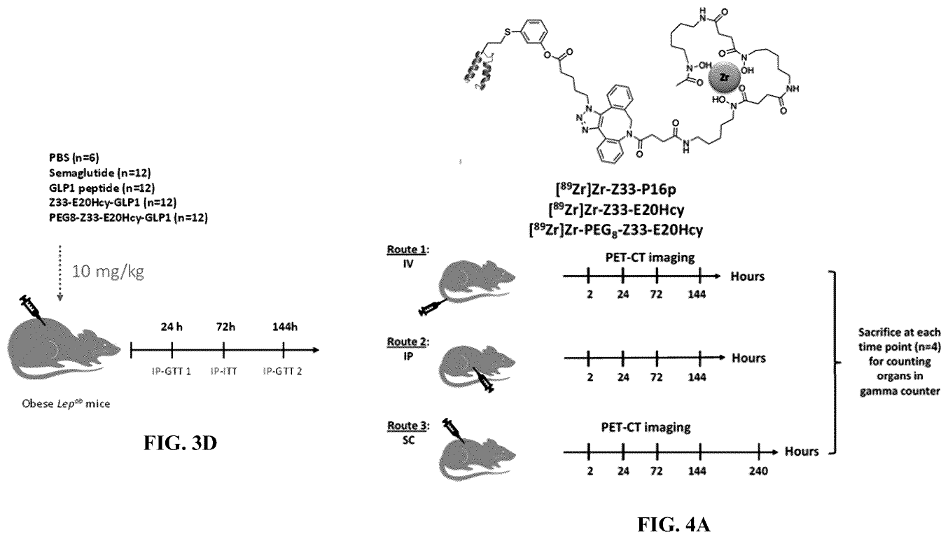

The ability of the IgG-binder electrophilic peptides to extend the PK/PD of GLP1 drugs was then evaluated. A cell experiment was first step up to evaluate GLP1 analog binding to the GLP1 receptor and evaluate the functionality of GLP1 after conjugation to IgG. After in vitro confirmation, an in vivo experiment was designed to evaluate GLP1 transfer to mouse IgG in wile-type Swiss mice. Mice were injected intraperitoneally with PBS (n=5), semaglutide (n=15), GLP1 peptide (n=15), Z33-E20Hcy-GLP1 (n=15), or pegylated-Z33-E20Hcy-GLP1 (n=15). An intraperitoneal glucose tolerance test (IP-GTT) was performed 24-, 72-, and 144 hours post-injection (FIG. 3A). Mice were injected with 20% dextrose and their blood glucose concentration was evaluated for up to 120 hours after glucose injection. Body weight follow-up was performed for 22 days after the single injection and demonstrated that the IgG-binder electrophilic peptides significantly decreased the percentage body weight from basal state over PBS or semaglutide alone (about 5-7%; FIG. 3B). These results demonstrate the ability of the IgG-binder electrophilic peptides to extend the PK/PD of GLP1 drugs up to 6 days for blood glucose control along with a body weight loss sustained for about 10 days after one single injection (versus 3 days for the commercial Semaglutide; FIG. 3C). Moreover, Z33-E20Hcy-GLP1 significantly slows down body weight intake up to 21 days after one single injection of 10 mg/kg compared to all of the controls and commercial Semaglutide.

The same study was repeated in obese mice models, which are deficient in leptin (Lep^{ob}) and present spontaneous type II diabetes and cardiometabolic diseases. Obese mice were injected peritoneally with PBS (n=6), semaglutide (n=12), GLP1 peptide (n=12), Z33-E20Hcy-GLP1 (n=12), or pegylated-Z33-E20Hcy-GLP1 (n=12). An intraperitoneal glucose tolerance test (IP-GTT) was performed 24-, 72-, and 144 hours post-injection (FIG. 3D).

TABLE 1
Toxicity profiles of GLP1 conjugates in WT Swiss mice. All GLP1 drugs were injected subcutaneously.
Every cohort contain 15 mice total, except for Semaglutide at 0.5 mg/kg and 3 mg/kg, and
Z33-E20Hcy-GLP1 stacking dose (3 × 10 mg/kg) which contain 5 mice each.

| | | | Number of mice with | | | | | | | |
|---|---|---|---|---|---|---|---|---|---|---|
| | | Mean | Hypoglycemia* | | | | | | | |
| | | basal | Mild | Moderate | Extreme | Number of mice with | | | | |
| | Dose | glycemia | (40-60 | (20-40 | (≤20 | Piloerection | | | | |
| Compound | (mg/kg) | (mg/dL)* | mg/dL) | mg/dL) | mg/dL) | Mild | Moderate | Severe | Aggressiveness | MTD |
| Semaglutide | 0.5 | 60.8 ± | 2 | 0 | 0 | 0 | 0 | 0 | 0 | 10 |
| (Ozempic ®) | | 9.44 | | | | | | | | mg/kg |
| | 3 | 61.4 ± | 1 | 1 | 0 | 0 | 2^{¤} | 3^{¤} | 5^{¤} | |
| | | 13.52 | | | | | | | | |
| | 10 | 40.4 ± | 1 | 2 | 1 | 0 | 0 | 15^{£} | 15^{£} | |
| | | 12.08 | | | | | | | | |
| GLP1 | 10 | 40.8 ± | 0 | 3 | 1 | 0 | 15^{¤} | 0 | 15^{¤} | 10 |
| peptide | | 11.76 | | | | | | | | mg/kg |
| PEG₈-Z33- | 10 | 82.4 ± | 0 | 0 | 0 | 0 | 0 | 0 | 0 | >10 |
| E20Hcy- | | 9.7 | | | | | | | | mg/kg |
| GLP1 | | | | | | | | | | |
| Z33- | 10 | 81.8 ± | 0 | 0 | 0 | 0 | 0 | 0 | 0 | ≥30 |
| E20Hcy- | | 9.44 | | | | | | | | mg/kg |
| GLP1 | 3 × 10, | 93.2 ± | 0 | 0 | 0 | 0 | 0 | 0 | 0 | |
| | over 3 | 11.4 | | | | | | | | |
| | weeks | | | | | | | | | |
| | 30 | 94.0 ± | 0 | 0 | 0 | 2* | 0 | 0 | 0 | |
| | | 4.7 | | | | | | | | |

*At 24 h post drug injection
^{¤}Over 48 h post injection
^{£}Over 72 h post injection

Mouse IgG Painting with Radionuclides

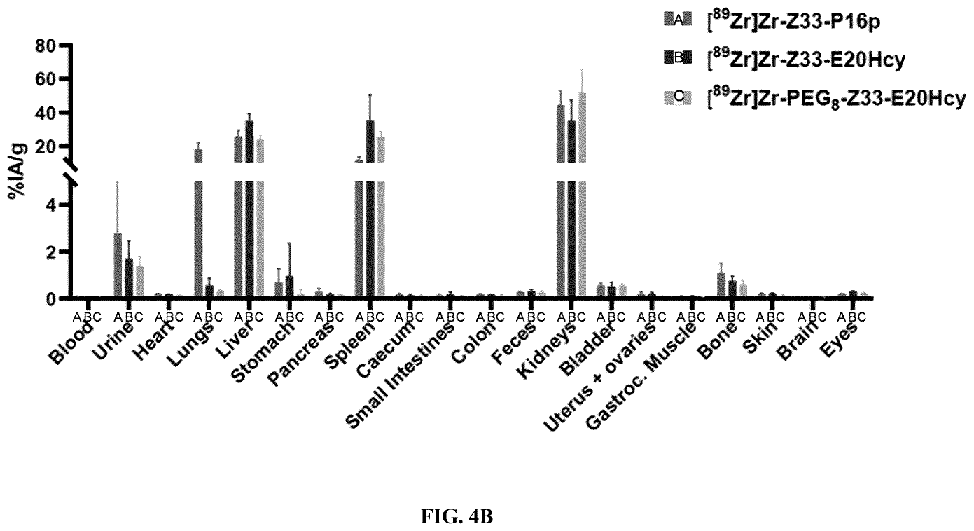

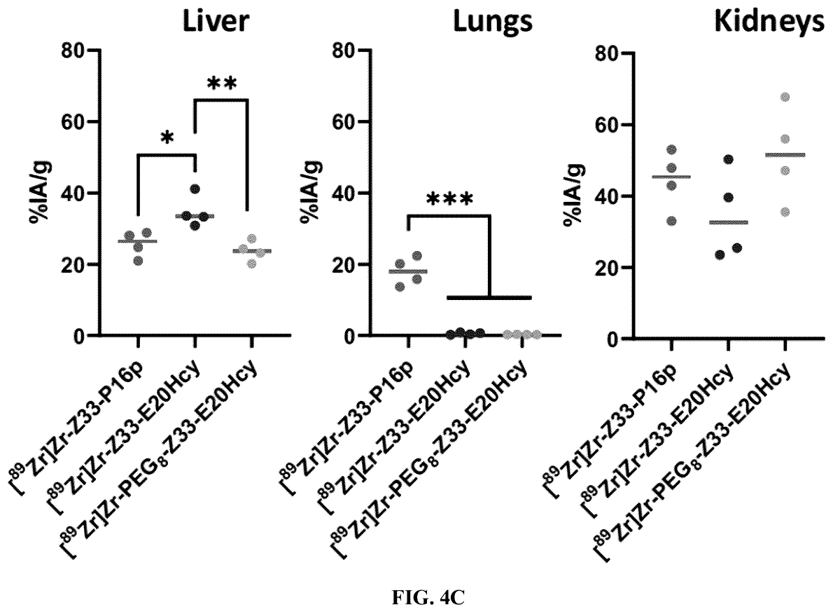

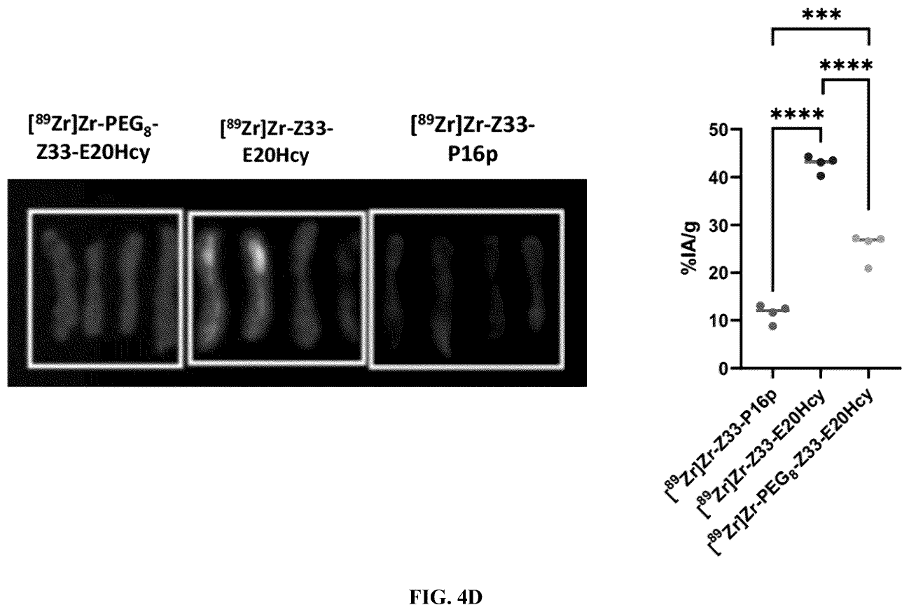

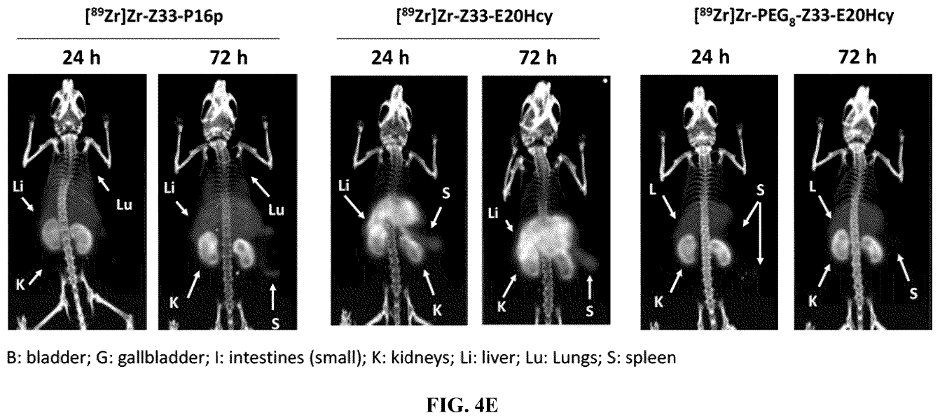

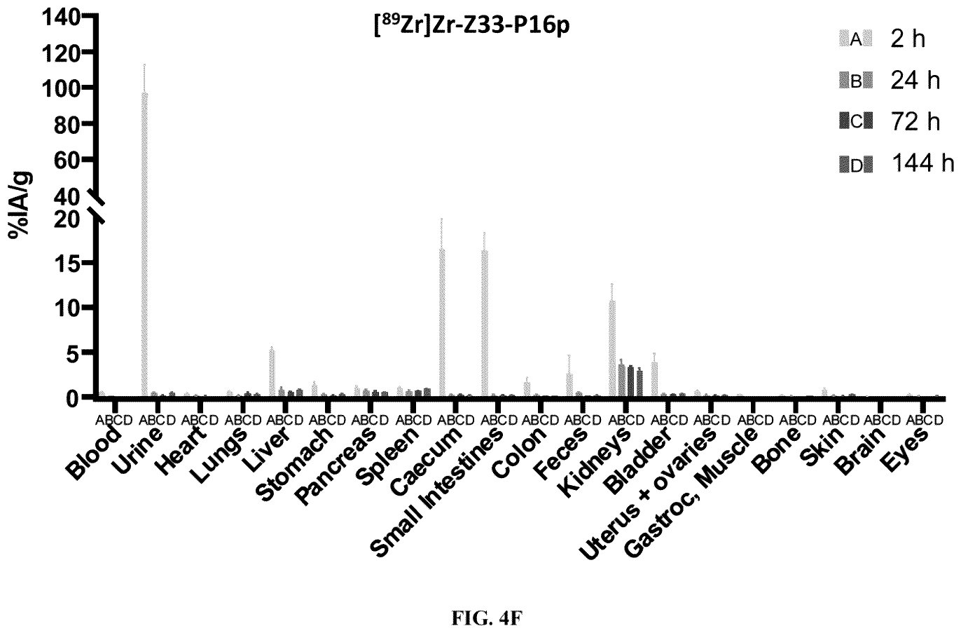

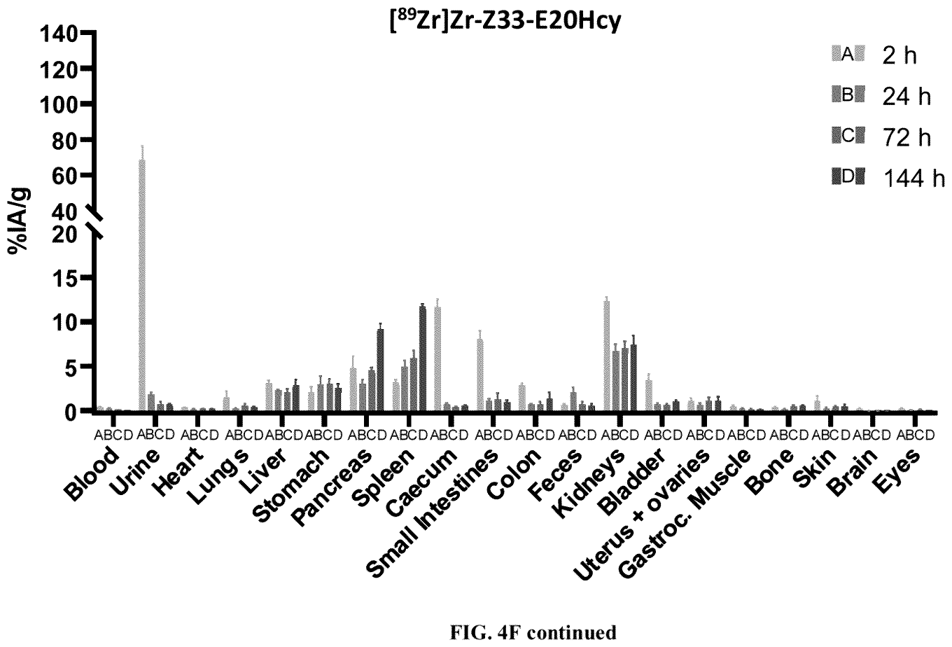

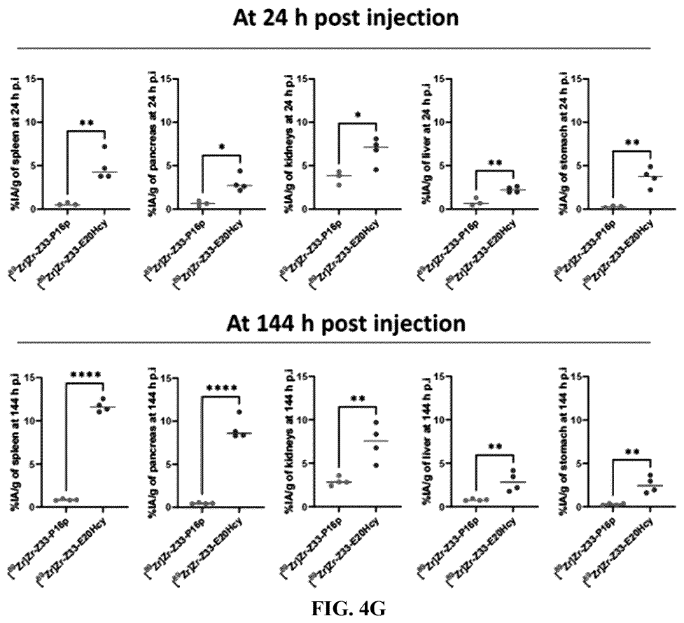

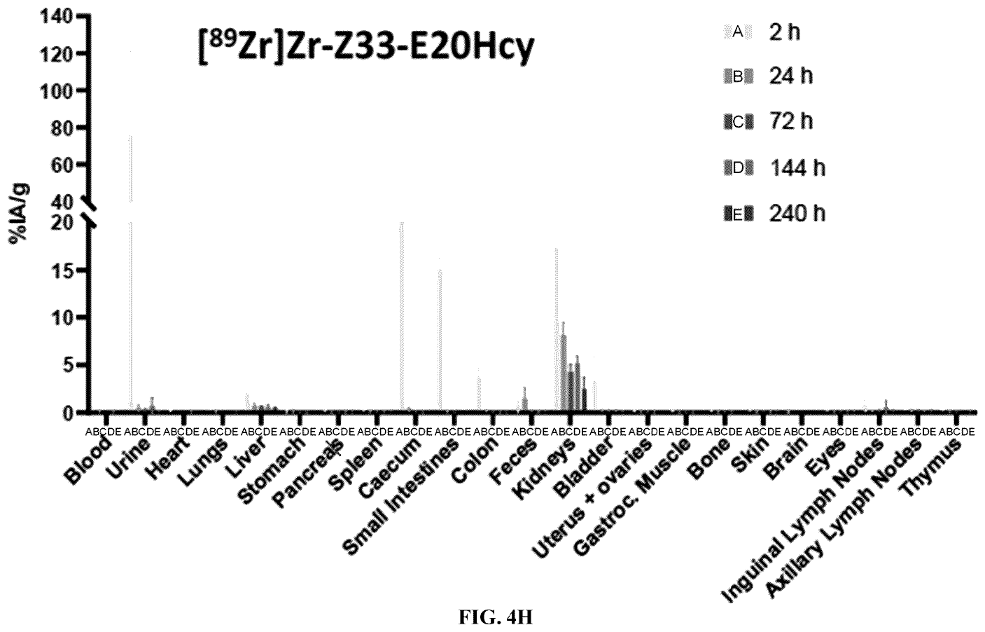

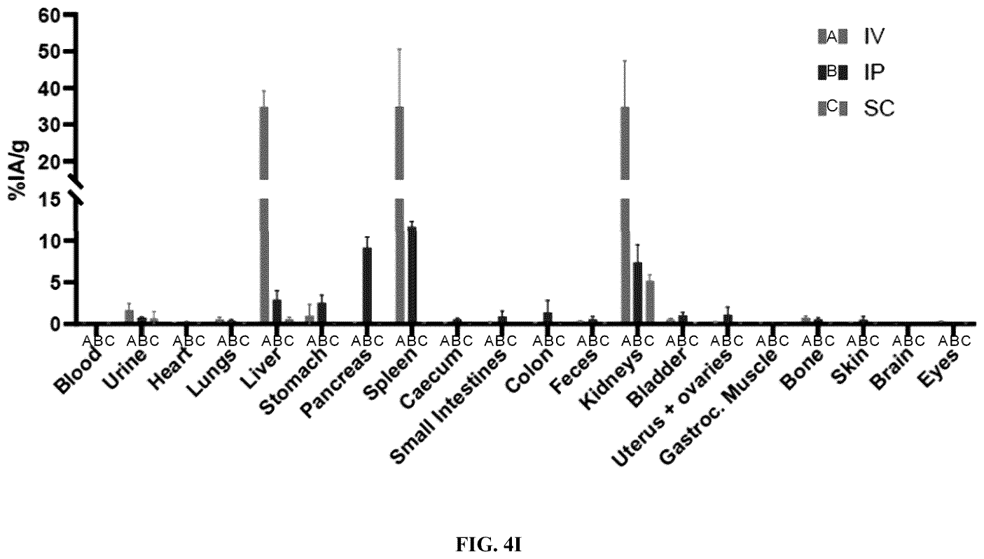

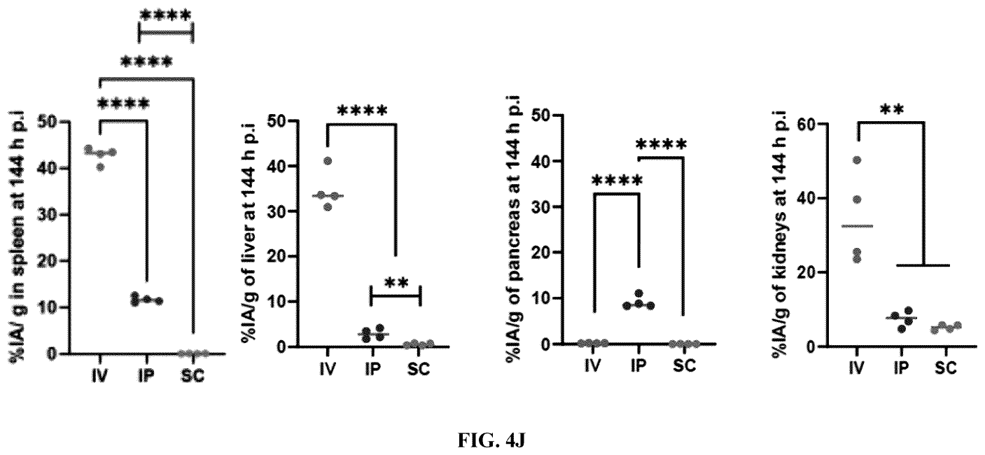

Further investigations using the most efficient electrophilic peptides highlighted that the IgG-binder electrophilic peptides could be capable of “painting” native antibodies (IgG1, IgG2, and IgG3) directly inside the body through the covalent transfer of either a small molecule, a radionuclide, or a bioactive long peptide. To evaluate this, wile-type Swiss mice were injected with about 1 MBq of Zr-89 labelled Z33-P16p, Z33-E20Hcy, or pegylated-Z33-E20Hcy intravenously, intraperitoneally, or subcutaneously (FIG. 4A). PET-CT imaging was performed at 2-, 24-, 72-, 144- and/or 240-hours post-injection. Additionally, 4 mice were sacrificed at each of these time points for determining the distribution in each organ using a gamma counter.

The whole blood biodistribution of the radiolabeled electrophilic peptides was evaluated at 144 hours post-intravenous injection (FIG. 4B). A detectable percentage of azido transfer was found in kidneys, spleen, liver, lungs, and urine. The injected activity was further compared between lungs, liver, and kidneys (FIG. 4C). No significant difference was found in the injected activity in the kidneys between the three electrophilic peptides. In the lungs, Z33-P16p had higher injected activity than both Z33-E20Hcy and pegylated-Z33-E20Hcy. In the liver, Z33-E20Hcy was found to have higher injected activity than Z33-P16p and pegylated-Z33-E20Hcy.

PET imaging and uptake in the spleen at 144 hours post-intravenous injection was also evaluated (FIG. 4D). Analysis of the percentage azido transfer demonstrated that Z33-E20Hcy had significantly higher activity than pegylated-Z33-E20Hcy, which in turn had significantly higher activity than Z33-P16p. PET-CT imaging was also done on the whole mouse body at 24- and 144-hours post-intravenous injection (FIG. 4E). These analyses confirmed sequestration of the electrophilic peptides to the kidneys, spleen, liver, and lungs.

Finally, a comparison between Z33-P16p and Z33-E20Hcy was performed. Whole-body biodistribution at 2-, 24-, 72-, and 144-hours post-injection showed markedly different biodistributions between the two electrophilic peptides (FIG. 4F). Z33-E20Hcy was found to be more evenly distributed across multiple organs, whereas Z33-P16p was primarily detected in the urine, kidneys, small intestines, and caecum. Across the spleen, pancreas, kidneys, liver, and stomach and at both 24-hours and 144-hours post-injection, Z33-E20Hcy was found to have significantly higher percentage azido transfer than Z33-P16p (FIG. 4G). As would be expected, the whole body biodistribution of Z33-E20Hcy decreases overtime, but was found to remain detectable up to 240 hours post-injection in the kidneys (FIG. 4H). When comparing the routes of administration at 144-hours post-injection, the percentage of azido transfer after injection with Z33-E20Hcy was found to be highest when administered intravenously than when administered intraperitoneally or subcutaneously (FIG. 4I). This was found to be significant in the spleen, liver, and kidneys but not the pancreas, where intraperitoneal injection was shown to result in significantly higher azido transfer activity than intravenous or subcutaneous routes of administration (FIG. 4J).

The transfer reaction is fully biocompatible, occurs without catalyzers, and does not require any biological engineering or production of antibody-drug conjugates beforehand as the payload is attached on native circulating IgGs that are already naturally produced by the organism. Taken together, these results show promising outcomes for making the next generation of weight loss and TDM drugs, and highlight the technology versatility, by enabling the transfer of not only bioactive peptides, but also potent chemicals. The IgG peptide-transfer technology represents a novel and powerful alternative for the manufacture of long-acting drugs.

### Example 2. Methods and Materials Materials

Fmoc-L-Ala-OH, Fmoc-L-Arg(Pbf)-OH, Fmoc-L-Asn(Trt)-OH, Fmoc-L-Asp-(Ot-Bu)-OH, Fmoc-L-Cys(Trt)-OH, Fmoc-L-Gln(Trt)-OH, Fmoc-L-Glu(Ot-Bu)-OH, Fmoc-L-Gly-OH, Fmoc-L-His(Trt)-OH, Fmoc-L-Ile-OH, Fmoc-L-Leu-OH, Fmoc-L-Lys(Boc)-OH, Fmoc-L-Met-OH, Fmoc-L-Phe-OH, Fmoc-L-Pro-OH, Fmoc-L-Ser(But)-OH, Fmoc-L-Thr(t-Bu)-OH, Fmoc-L-Trp(Boc)-OH, Fmoc-L-Tyr(t-Bu)-OH, and Fmoc-L-Val-OH were purchased from Novabiochem, Millipore Sigma, or Chem-Impex Inc. Fmoc-alpha-methylalanine (Fmoc-Aib-OH) and Fmoc-L-HomoCys(Trt)-OH (Fmoc-Hcy-OH) were purchased from Combi-Blocks Inc. 5-azidopentanoic acid was purchased from ChemPep®. 1-(9H-Fluoren-9-yl)-3-oxo-2,7,10,13,16,19,22,25,28-nonaoxa-4-azahentriacontan-31-oic acid (Fmoc-PEG8-CO2H) was purchased by AmBeed. O-(7-azabenzotriazol-1-yl)-N,N,N′,N′-tetramethyluronium hexafluorophosphate (HATU), and (7-azabenzotriazol-lyloxy)tripyrrolidinophosphonium hexafluorophosphate (PyAOP) were purchased from P3 Biosystems. H-Rink Amide-ChemMatrix resin was purchased from PCAS BioMatrix Inc. Polyethylene filter paper for peptide synthesis (0.60 mm thick, pore size 7-12 μm) was purchased from Interstate Specialty Products. N,N-dimethylformamide (OmniSolv® for Biosynthesis, DMF, stored with an AldraAmine trapping packet), diethyl ether (≥99.0% stabilized by BHT, Et2O), acetonitrile (≥99.9%, CH3CN), dichloromethane (≥99.8% stabilized by amylene, CH2Cl2), 1,4-dioxan (≥99.5% stabilized by BHT), 2-propanol (99.9%), 2-methyl-tetrahydrofuran (≥99.5% stabilized by BHT, 2-Me-THF), dimethylsulfide (≥99.5%, BioUltra for molecular biology, DMSO), and pentane (98% reagent grade) were obtained from Millipore-Sigma. Ethyl acetate (≥99.5%), hexane (≥98.5%, mixture of isomers), Methanol (≥99.8%), and acetone (≥99.5%) were purchased from VWR. The water used in all reactions on proteins, in the preparation of buffers, and in the preparation of mobile phases for purification was obtained via filtration of deionized water through a Millipore Sigma Milli-Q™ Ultrapure Water System. AldraAmine trapping packets and piperidine were purchased from Millipore Sigma. Amine-free DMF refers to DMF stored over AldraAmine trapping packets for a minimum of 24 hours prior to use. LC/MS grade water, acetonitrile, and formic acid were purchased from Thermo Fisher Scientific Inc and were used for liquid chromatography-mass spectrometry (LC-MS). All small compounds were purchased from TCI America, Millipore Sigma, AmBeed, Matrix Scientific, or Combi-Blocks in purities ≥95%, and were used without further purification (unless otherwise noted). PBS buffer (1× and 10×) were purchased from CORNING®. Reactions monitored by analytical thin-layer chromatography (TLC) were carried out using glass-backed plates pre-coated with silica gel impregnated with a fluorescent indicator (254 nm). All deuterium solvents were purchased from Cambridge Isotope Laboratories, Inc. C18-ZipTips (0.6 μL) were purchased from Millipore Sigma. Zeba spin desalting columns were purchased from Thermo Fisher Scientific Inc. Trastuzumab and its biosimilar were purchased from Syd Labs Inc., MedChemExpress LLC., and Bio X Cell.

### NMR

NMR spectra were recorded on Bruker Avance-III HD 400 MHz spectrometer. All 1H NMR chemical shifts are expressed in parts per million (ppm, δ scale) and are referenced to the residual proton in the NMR solvent (CDCl3-d3: 7.26, DMF-d7: 8.03, DMSO-d6: 2.50). All 13C spectra recorded are proton decoupled. The 13C NMR chemical shifts are expressed in part per million (ppm, δ scale) and are referenced to the carbon resonance of the NMR solvent (CDCl3-d3: 77.16, DMF-d7: 163.15, DMSO-d6: 39.52). All 31P chemical shifts are expressed in parts per million (ppm, δ scale). 1H NMR spectroscopic data are reported as follows: a chemical shift in ppm (multiplicity, coupling constants J (Hz), integration intensity, assigned number of protons in molecule). The multiplicities are abbreviated with s (singlet), br. s (broad singlet), d (doublet), t (triplet), q (quartet), and m (multiplet). In the case of combined multiplicities, the multiplicity with the larger coupling constant is stated first. The chemical shift of all signals is reported as the center of the resonance range, except in the case of multiplets, which are reported as ranges in chemical shift. All raw fid files were processed, and the spectra analyzed using the program MestReNova 14.2.3 from Mestrelab Research S. L.

### Liquid Chromatography-Mass Spectrometry (LC-MS) Using Quadrupole Time-of-Flight (Q-ToF)

LC-MS chromatograms and associated mass spectra were acquired using an Agilent Technologies 6550 Q-ToF LC-MS system. Solvent compositions are 0.1% formic acid in H2O (solvent A) and 0.1% formic acid in acetonitrile (solvent B). For methods A, B, and C, a calibration solution containing mass 922.009798 constantly flowed through the system. The following LC-MS methods were used:

Q-ToF LC-MS Method A: LC conditions: Zorbax 300SB C3 column: 2.1×150 mm, 5 μm, column temperature: 40° C., gradient: 0-1 minutes 1% B, 1-6 minutes 1-61% B, flow rate: 0.8 mL/minute. MS conditions: positive electrospray ionization (ESI) extended dynamic mode in mass range 300-3000 m/z, temperature of drying gas=350° C., flow rate of drying gas=11 L/minute, pressure of nebulizer gas=60 psi, the capillary, fragmentor, and octapole voltages were set at 4000, 175, and 750 V, respectively.

Q-ToF LC-MS Method B: LC conditions: Zorbax 300SB C3 column: 2.1×150 mm, 5 μm, column temperature: 40° C., gradient: 0-1 minutes 1% B, 1-7 minutes 1-91% B, flow rate: 0.7 mL/minute. MS conditions: positive electrospray ionization (ESI) extended dynamic mode in mass range 300-3000 m/z, temperature of drying gas=350° C., flow rate of drying gas=11 L/minute, pressure of nebulizer gas=60 psi, the capillary, fragmentor, and octapole voltages were set at 4000, 175, and 750 V, respectively.

Q-ToF LC-MS Method C: LC conditions: Zorbax 300SB C3 column: 2.1×150 mm, 5 μm, column temperature: 40° C., gradient: 0-1 minutes 1% B, 1-6 minutes 1-41% B, flow rate: 0.8 mL/minute. MS conditions: positive electrospray ionization (ESI) extended dynamic mode in mass range 300-3000 m/z, temperature of drying gas=350° C., flow rate of drying gas=11 L/minute, pressure of nebulizer gas=60 psi, the capillary, fragmentor, and octapole voltages were set at 4000, 175, and 750 V, respectively.

LC-MS Method D: LC conditions: Aeris WIDEPORE C4 200 column: 2.1×150 mm, 3.6 μm, column temperature: 40° C., gradient: 0-2 minutes 1% B, 2-8 minutes 1-91% B, 8-10 minutes 91-95% B, flow rate: 0.3 mL/minute. MS conditions: positive electrospray ionization (ESI) extended dynamic mode in mass range 100-1700 m/z, temperature of drying gas=200° C., flow rate of drying gas=14 L/minute, pressure of nebulizer gas=55 psi, the capillary, fragmentor, and octapole voltages were set at 3500, 175, and 750 V, respectively.

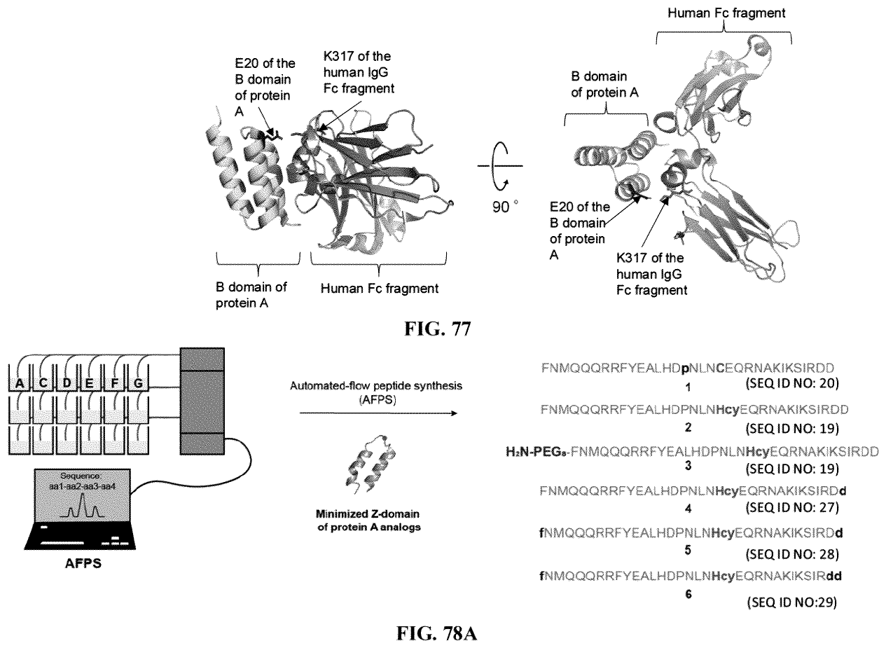

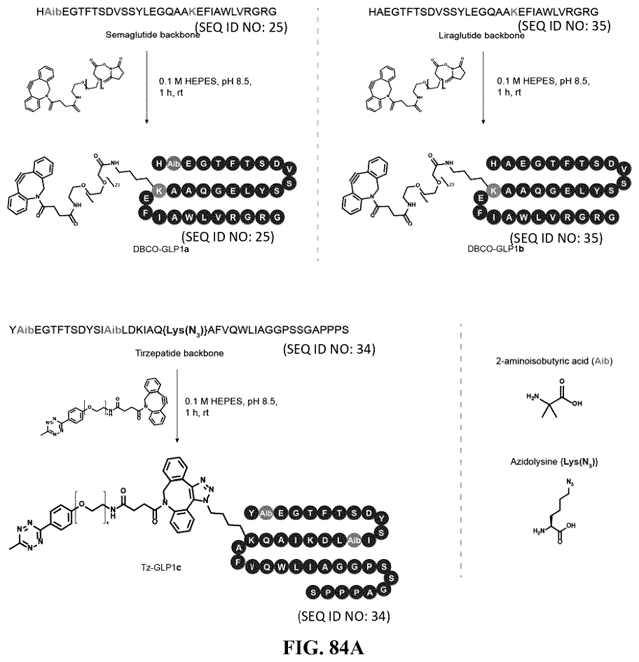

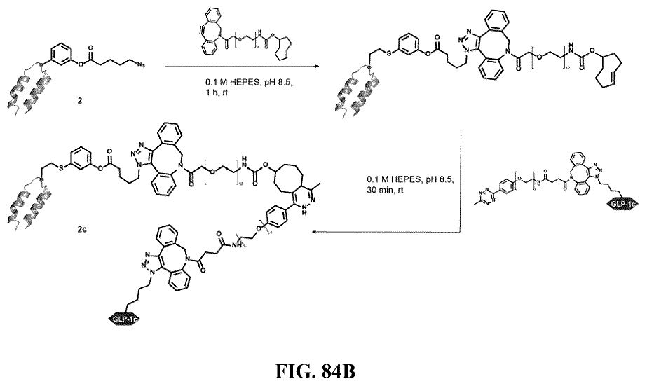

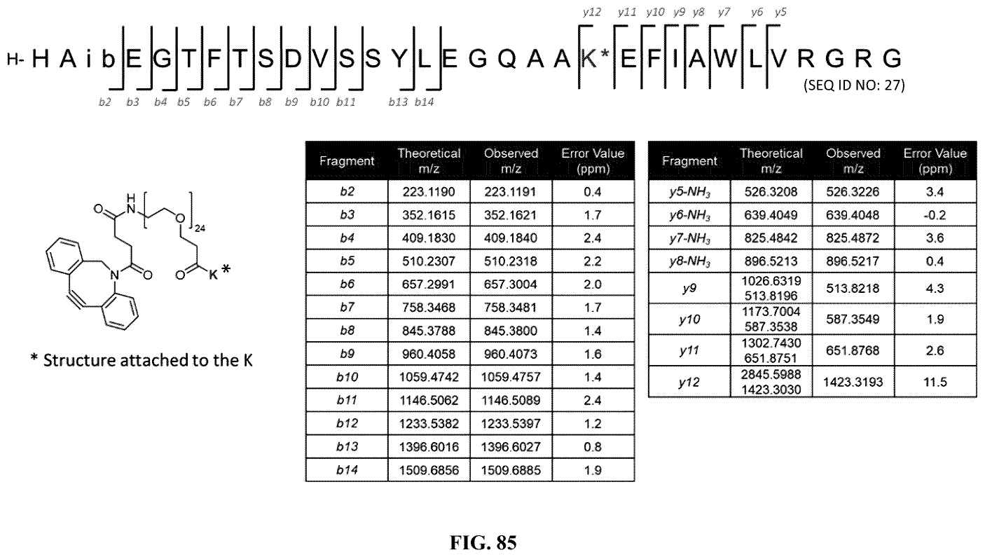

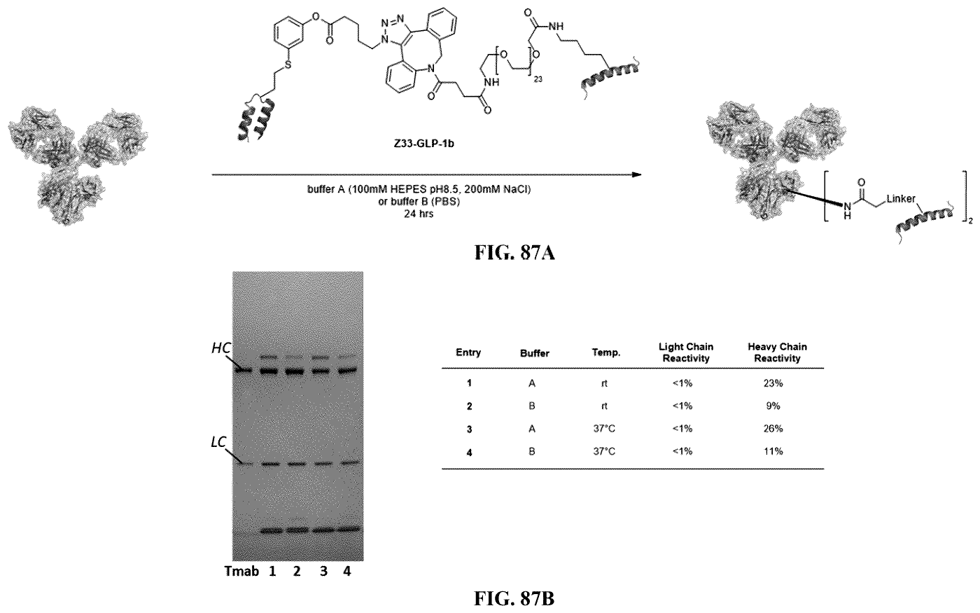

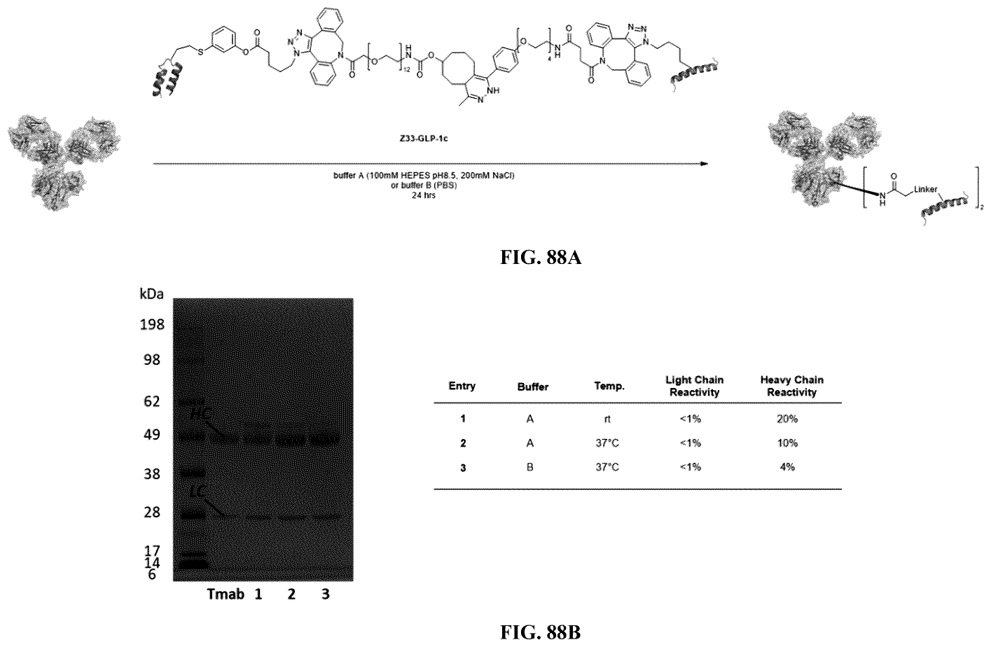

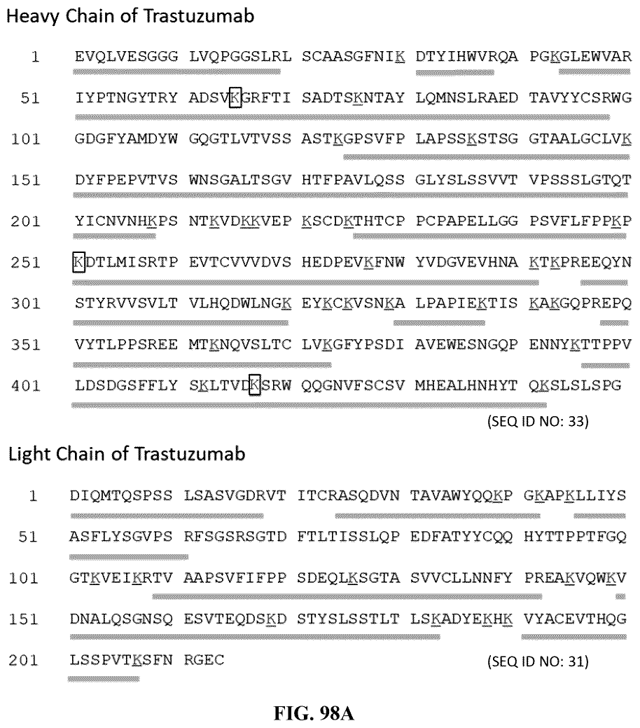

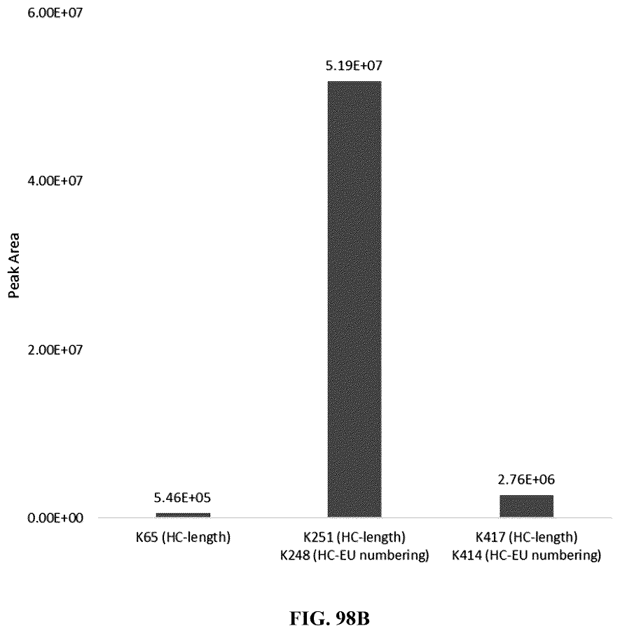

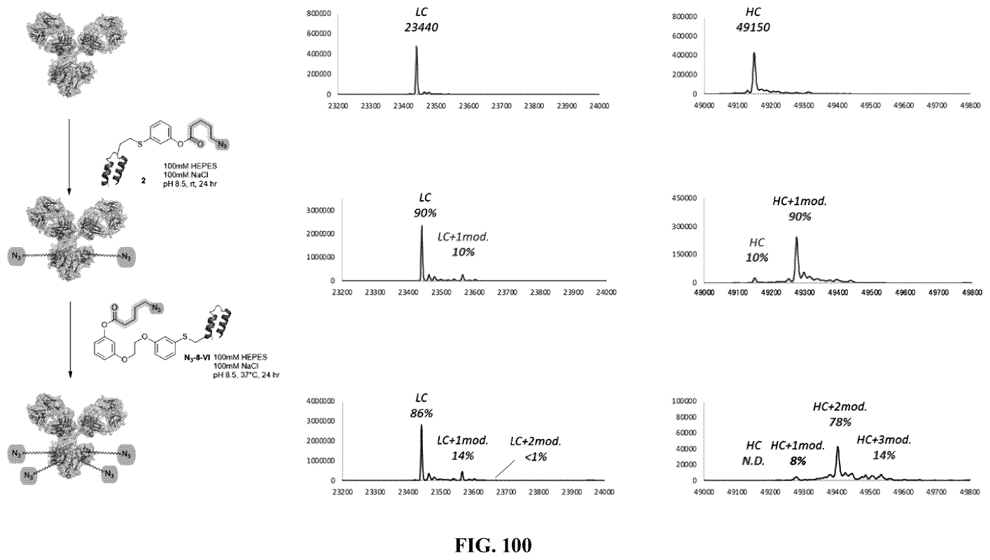

Data were processed using Agilent MassHunter Workstation Qualitative Analysis Version B.06.00 Software or Agilent MassHunter BioConfirm Software B.10.00. Deconvoluted masses of proteins were obtained using a maximum entropy algorithm. Unless otherwise depicted, the following parameters were used for deconvolution: Mass range is given in the experimental section for each peptide; Mass step was set to 1.00 Daltons; Baseline was set to Subtract baseline, Baseline factor as 7.00. The y-axis of all chromatograms shown in the FIGS. 77 to 102B represent the total ion current (TIC), and the inset of the mass spectrum corresponds to the deconvolution of the entire protein including peaks.

### LC-MS Using Single Quadrupole Mass Spectrometry

Mass spectra were obtained on an Agilent 6125B mass spectrometer attached to an Agilent 1260 Infinity LC. Solvent compositions are 0.1% formic acid in H2O (solvent A) and 0.1% formic acid in acetonitrile (solvent B). The following LC-MS method was used:

LC conditions: Poroshell 120 SB C18: 2.1×50 mm, 2.7 μm, column temperature: 40° C., gradient: 0-1 minutes 10% B, 1-5 minutes 10-100% B, 5-6 minutes 100% B, 6-7 minutes 100-10% B, flow rate: 0.4 mL/minute. MS conditions: positive electrospray ionization (ESI) extended dynamic mode in mass range 100-1500 m/z, temperature of drying gas=350° C., flow rate of drying gas=13 L/minute, pressure of nebulizer gas=35 psi, the capillary, fragmentor, and octapole voltages were set at 4000, 70, and 650 V, respectively.

Nano-Liquid Chromatography-Tandem Mass Spectrometry (nLC-MS/MS)

Analysis was performed on an EASY-nLC 1200 nano-liquid chromatography system connected to an Orbitrap Fusion Lumos Tribrid Mass Spectrometer or to an Orbitrap Fusion Eclipse Tribrid Mass Spectrometer (Thermo Fisher Scientific). Samples were run on a PepMap RSLC C18 column (C18, 50 um×15 cm, 2 um, 100 Å, Thermo Fisher Scientific, P/N ES901). An Acclaim PepMap 100 Trap column (C18, 75 um×2 cm, 3 um, 100 Å, Thermo Fisher Scientific, P/N 164946) was used for desalting. Solvent compositions are 0.1% formic acid in H2O (solvent A) and 0.1% formic acid in 80% acetonitrile and 19.9% H2O (solvent B). The following conditions were used for each sample measurement:

nLC-MS/MS Method A: LC conditions: column temperature: 40° C., gradient: 0-30 minutes 30-95% B, 30-40 minutes 95% B, flow rate: 300 nL/minute. MS conditions: positive ion spray voltage was set to 2200 V. Orbitrap detection was used for primary MS, with the following parameters: Application mode=peptide, cycle time=3 s, resolution=120000, mass range=normal, scan range=250-2000 m/z, RF Lens=30%, AGC target=standard, maximum injection time mode=auto, 1 microscan, data type-profile, polarity=positive, The following filters were applied for MS2 precursor selection: monoisotopic peak determination=peptide, filter type=threshold, Intensity threshold=5.0e5, include charge state=2-6, mass list type=m/z, target mass*, mass tolerance=25 ppm. Fragmentation was induced by higher-energy collisional dissociation (HCD) and electron-transfer dissociation with higher-energy collision (EThcD). Specifications HCD: isolation mode=quadrupole, isolation window=1.2 m/z, isolation offset=0.6 m/z, activation type=HCD, collision energy mode=fixed (30%), detection type=orbitrap, resolution=30000, mass range=normal, first mass=120 m/z, AGC target=standard, maximum injection time mode=dynamic, 1 microscan, data type=profile. Specifications EThcD: isolation mode=quadrupole, isolation window=1.2 m/z, isolation offset=0.6 m/z, activation type=ETD, use calibrated charge-dependent ETD parameters, ETD supplemental activation=EThcD, SA collision energy=30%, detection=orbitrap, resolution=30000, mass range=normal, first mass=120 m/z, normalized AGC target=standard, maximum injection time=dynamic, 1 microscans, data type=profile. A list of target masses was created by calculating the intrinsic masses of the measured samples.

nLC-MS/MS Method B: LC conditions: column temperature: 40° C., gradient: 0-30 minutes 1-10% B, 30-120 minutes 10-81% B, 120-125 minutes 81-90% B, 125-135 minutes 90% B, flow rate: 300 nL/minute. MS conditions: positive ion spray voltage was set to 2200 V, and negative ion spray voltage was set to 600 V. Orbitrap detection was used for primary MS, with the following parameters: Application mode=peptide, cycle time=3 s, resolution=120000, mass range=normal, scan range=200-1400 m/z, RF Lens=30%, AGC target=custom, normalized AGC target (%)=250, maximum injection time mode=auto, 1 microscan, data type=profile, polarity=positive, The following filters were applied for MS2 precursor selection: monoisotopic peak determination=peptide, exclude after 4 times, If occurs within 30 seconds, exclusion duration=30 seconds, mass tolerance=10 ppm, exclude isotope, include charge state=2-10, filter type=threshold, Intensity threshold=5.0e4, precursor selection range=200-1400, Fragmentation was induced by collision-induced dissociation (CID), higher-energy collisional dissociation (HCD), and electron-transfer dissociation with higher-energy collision (EThcD). Specifications CID: isolation mode=quadrupole, isolation window=1.3 m/z, activation type=CID, collision energy mode=fixed (30%), CID activation time=10 milliseconds, activation Q=0.25, detection type=orbitrap, resolution=30000, mass range=normal, AGC target=custom, normalized AGC target=40%, maximum injection time mode=auto, 1 microscan, data type=centroid. Specifications HCD: isolation mode=quadrupole, isolation window=1.3 m/z, activation type=HCD, detection type=orbitrap, resolution=30000, mass range=normal, scan range mode=auto, AGC target=custom, normalized AGC target=40%, maximum injection time mode=auto, 1 microscan, data type=centroid. Specifications EThcD: isolation mode=quadrupole, isolation window=1.3 m/z, activation type=ETD, use calibrated charge-dependent ETD parameters, ETD supplemental activation=EThcD, SA collision energy=25%, detection=orbitrap, resolution=30000, mass range=normal, scan range mode=auto, normalized AGC target=40%, normalized AGC target=custom, maximum injection time=auto, 1 microscans, data type=profile.

### Ultra High-Performance Liquid Chromatography (UHPLC)

The samples were analyzed using an Agilent Technologies 1290 Infinity II LC system which was computer-controlled through Agilent ChemStation software. The following methods were used:

UHPLC Method A (for peptide/protein analysis excluding IgG): Solvent compositions used in the UHPLC are 0.1% TFA in H2O (solvent A) and 0.1% TFA in acetonitrile (solvent B). ACQUITY UPLC Protein BEH C4 column: 2.1×50 mm, 1.7 μm (Waters, P/N 186004495), ACQUITY UPLC Protein BEH C4 VanGurde pre-column: 2.1×5 mm, 1.7 μm (Waters, P/N 186004623), column temperature: 27° C., gradient: 0-3 minutes 1% B, 3-13 minutes 1-61% B, flow rate: 0.5 mL/minute.

UHPLC Method B (For IgGs analysis): Solvent compositions used in the UHPLC are 0.1% TFA in 98% H2O and 2% 2-propanol (solvent A) and 70% 2-propanol, 20% acetonitrile, and 10% solvent A (solvent B). AdvanceBio RP-mAb Diphenyl USP L11 column: 2.1×150 mm, 3.5 μm (Agilent, P/N 793775-944), column temperature: 80° C., gradient: 0-1 minutes 15% B, 1-3 minutes 15-30% B, 3-15 minutes 30-40% B, 15-16 minutes 40-60% B, 16-20 minutes 60-15% B, flow rate: 0.5 mL/minute.

### Automated Liquid Chromatography Purification

A Biotage® Selekt automated flash chromatography system was used for the purification of small molecules and peptides. The columns, solvents, and solvent gradients employed for the purification of individual compounds are written in the synthesis section of each compound. Sodium Dodecyl Sulfate-polyacrylamide Gel Electrophoresis (SDS-PAGE)

Bolt 4-12% Bis-Tris Plus 1.0 mm×15 well or 1.0 mm×10 well plates (Invitrogen), Mini Gel Tank (Invitrogen) along with the PowerPac HC (BIO-RAD) were used for SDS-PAGE analysis. SeeBlue® Plus2 standard (Invitrogen) was used as the molecular weight standard. Gels were run using Bolt MOPS SDS Running Buffer (1×, Invitrogen) under the conditions of 135V for 55 minutes. After electrophoresis, the running buffer was discarded, and the gel was placed in deionized water. Subsequently, the water with gel was heated in a microwave for 1 minute and seconds. The heated water was replaced, and the gel was thoroughly rinsed. Gel staining was performed for 10 minutes using SimpleBlue SafeStain (Invitrogen). The stain was removed following staining, and the gel was immersed in deionized water. The tray with gel and deionized water was placed on a shaker overnight to remove the stain from the gels. Then, ChemDoc MP Imaging System (BIO-RAD) was used to analyze the stained gels.

### Determination of the Reaction Conversion of IgGs

The conversion rate for each reaction was calculated by one of the following methods. Determination by LC-MS: Reported yields based on LC-MS spectra were determined by extracting the total ion current (TIC) spectra of all protein-containing species in the chromatogram utilizing Agilent MassHunter 6.0 software with BioConfirm package. These extracted chromatograms were deconvoluted utilizing a maximum entropy algorithm and abundance of each species determined using total ion count.

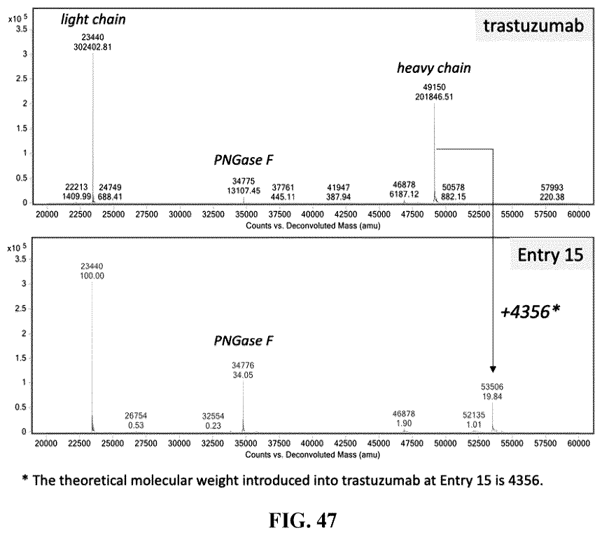

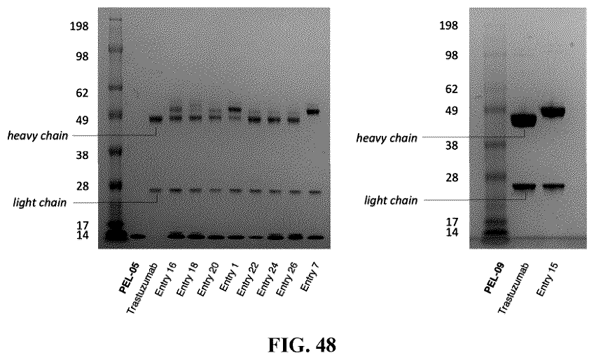

Determination by SDS-PAGE: Band densitometry was calculated using ImageJ software. Densitometry-based yields were calculated based on the product: starting material protein ratio in lanes and standardized based on molecular weight. An example is shown in FIG. 47. After plotting bands and integration using ImageJ software, the value of each band was obtained as 7122.93 and 10408.80, respectively.
Starting material: right peak standardization (MW 49151) 10408.80/49151=0.21177
Modified product: left peak standardization (MW 53381) 7122.93/53381=0.13344
Thus, the determined conversion (%) would be Standardized Product/(Standardized Total): 0.13344/(0.21177+0.13344)*100=39%

### Animal Experiments

All animal experiments were performed under an MIT institute approved IACUC protocol following federal, state, and local guidelines for the care and use of animals (protocol number #0821-058-24). The studies were performed on 30-35 g (5-8 weeks-old) healthy female Swiss mice, purchased from The Jackson Laboratory (MA, USA). Mice were housed with free access to food and water ad libitum unless stated otherwise for the need of the experiment. For subcutaneous (SC) injections, the mice were shortly anesthetized beforehand with 2-3% isoflurane along with O₂, then shaved on the right flank. For intravenous injections (IV), the mice were anesthetized with 2-3% isoflurane along with O₂, then a catheter was inserted in the lateral tail vein to ensure the injection was properly done. Intraperitoneal injections (IP) were performed on vigil mice, in the lower right abdominal quadrant. For blood collection, mice were either punctured in the heart (terminal procedure), or gently pressed at the tail following tail pricks (vigil). For multiple blood collections following the injection of radionuclides, the mice were first anesthetized with 2-3% isoflurane along with O₂, before performing tail pricks, for radiation safety matter. The animals were sacrificed by CO₂ inhalation followed by cervical dislocation. All compounds injected in mice were either USP grade, sterile, or filtered using 0.22 μm.

### Example 3. Chemical Synthesis of IgG Binding Peptide Z33 Analogs

### Automated Flow-Based Peptide Synthesis System

The synthesis of the IgG binding peptide Z33 (Proc. Natl. Acad. Sci. U.S.A. 93, 5688-5692 (1996)) analogs were performed using the Automated-flow solid phase peptide synthesis (AFPS) system (Science, 368, 980-987 (2020)). The synthesis conditions are summarized in

TABLE 2
IgG binding peptide Z33 analog synthesis conditions.

| Parameter | Conditions |
|---|---|
| temperature | 85-90° C. in reactor, 60° C. in 5′ activation loop (for C |
| | and H), 90° C. in 10' activation loop (for all other |
| | amino acids) |
| Flow Rate | 40 mL/min |
| Coupling Step | 0.40 M amino acids stocks in DMF |
| | 0.38 M activator stocks in DMF |
| | Coupling conditions: HATU (13 pump strokes) except |
| | S and A with HATU (26 pump strokes) and C, H, N, |
| | Q, V, R, T with PyAOP (26 pump strokes) |
| Deprotection | 40% piperidine in DMF with 2% formic acid (13 pump |
| Step | strokes) |
| Washing Steps | DMF (40 pump strokes) |

### Synthesis of Z33 Variants Composed of Natural Amino Acids

A plastic fritted syringe (6 mL) was equipped with polyethylene filter paper, and ChemMatrix® H-Rink Amide resin (0.49 mmol/g, 150 mg) was loaded onto it. The syringe assembly was then placed on a manifold. The resin was swollen in DMF, and subsequently, a homogeneous resin slurry was prepared by repeatedly drawing back 500 μL of the slurry using a 1 mL pipette. Next, the syringe was set to the AFPS system for peptide synthesis. After completion of the synthesis, the syringe was transferred from the AFPS system to the manifold and washed three times with CH2C12. To minimize methionine oxidation in the peptide sequence, drying of the resin was not conducted using air flow on the manifold but rather using nitrogen flow. Specifically, the syringe was capped with a rubber septum, and a needle connected to a nitrogen flow line was inserted into the septum for 15 minutes. The resulting peptide-bound dry resin was subjected to subsequent steps of cleavage and purification.

### Synthesis of Z33 Variants Containing Unnatural Amino Acids

The peptide was synthesized using the method described in the “Synthesis of Z33 Variants Composed of Natural Amino Acids” section from the C-terminus of the sequence to one unnatural amino acid before the sequence. The resin in a plastic fritted syringe was transferred from the AFPS system to a manifold. Fmoc-protected unnatural amino acid (10 equivalents for the peptide on the resin) in HATU/DMF (0.38 M, 9.5 equivalents) solution and DIEA (15 equivalents) were added to the resin and allowed to react for 50 minutes at room temperature.

The reaction solution was stirred with a spatula every 10 minutes. After the reaction, the reaction solution was removed, and the resin in the syringe was washed three times with 5 mL of DMF. Then, 3 mL of 40% piperidine/DMF solution (+2% formic acid) was added and allowed to react for 10 minutes. The reaction solution was removed, and 3 mL of 40% piperidine/DMF solution (+2% formic acid) was added again and allowed to react for 10 minutes. The reaction solution was removed, and the resin was washed three times with 4 mL of DMF. Subsequent synthesis of the sequence using natural amino acids was again performed according to the procedure previously described.

### Capping of the N-Terminus of Z33 Variants

After synthesis of the peptide by AFPS, the fritted syringe with resin was placed on a manifold. A HATU/DMF solution (0.38 M, 9.5 equivalents) of the carboxylic acid corresponding to the acyl group (10 equivalents for the peptide on the resin) and DIEA (15 equivalents) were added and allowed to react for 50 minutes at room temperature. During that time, the reaction solution was stirred with a spatula every 10 minutes. After the reaction, the reaction solution was removed, and the resin in the syringe was washed three times with 10 mL of DMF. If the introduced acyl group contained the Fmoc protecting group, 3 mL of 40% piperidine/DMF solution (+2% formic acid) was added and allowed to react for 10 minutes. The reaction solution was removed, and 3 mL of 40% piperidine/DMF solution (+2% formic acid) was added again and allowed to react for 10 minutes. The reaction solution was removed and washed three times with 4 mL of DMF and three times with CH₂Cl₂. To minimize methionine oxidation in the peptide sequence, the resin was not dried using airflow on the manifold but rather using nitrogen flow. Specifically, the syringe was capped with a rubber septum, and a needle connected to a nitrogen flow line was inserted into the septum for 15 minutes. The resulting peptide-bound dry resin was subjected to subsequent steps of cleavage and purification.

Cleavage of Z33 Variants from Resin

The peptide-bound resin was transferred to a 15 mL centrifuge tube. Subsequently, a 7.5 mL peptide cleavage solution (a mixture of TFA, water, EDT, and TIPS in a ratio of 94:2.5:2.5:1) was added to the centrifuge tube, and the reaction was allowed to proceed at room temperature for 2 hours on the shaker. Afterward, 5 mL of a plastic fritted syringe was placed on top of a 50 mL centrifuge tube, and the reaction solution containing the resin was filtered. The resin inside the syringe was washed twice with 3 mL of TFA. To the resulting solution, diethyl ether pre-cooled at −80° C. was added until a total volume of 45 mL was reached. The centrifuge tube was then capped, and the reaction solution was vortexed for 5 seconds, followed by centrifugation at 3220 rcf (relative centrifugal force) for 4 minutes. Subsequently, the supernatant was removed by decantation. The obtained crude peptide was dissolved in 10 mL of 50:50 water and acetonitrile solution (+0.1% TFA), followed by freezing the solution with liquid nitrogen and subjecting it to lyophilization. When the peptide contained the azido moiety, another peptide cleavage solution (a mixture of TFA, water, thioanisole, and TIPS in a ratio of 94:2.5:2.5:1) was used instead of the solution described above.

Purification of the Z33 Variants from Resin

The crude Z33 analog was dissolved in a 95:5 water-acetonitrile solution and loaded onto 12 g of Biotage® Sfär C18 column. The gradient used for purification was as follows: 3 CV (column volume) 5% B, 1 CV 5-10% B, 30 CV 10-40% B. Fractions containing the desired product were collected in 50 mL centrifuge tubes, and the solution was frozen using liquid nitrogen. The target Z33 variant was obtained as white solids by removing the solvent through lyophilization. The purified Z33 analogs are characterized in Table 3

TABLE 3
Synthesized Z33 analogs.

| | | | SEQ | | | |
|---|---|---|---|---|---|---|
| | | | ID | | Calculated | Observed |
| Code | N-term | Sequence | NO: | C-term | Mass^{a} | Mass^{b} |
| PEP-01 | Free | CFNMQQQRRFYEALHDPNLNEEQRNAKIKSIRDD | 11 | Amide | 4207.7 | 4207.7 |
| PEP-02 | Free | FNCQQQRRFYEALHDPNLNEEQRNAKIKSIRDD | 12 | Amide | 4076.5 | 4077.1 |
| PEP-03 | Free | FNMQQQRRFYEALHDPCLNEEQRNAKIKSIRDD | 13 | Amide | 4093.6 | 4093.7 |
| PEP-04 | Free | FNMQQQRRFYEALHDPNLNCEQRNAKIKSIRDD | 14 | Amide | 4078.6 | 4078.6 |
| PEP-05 | Free | FNMQQQRRFYEALHDPNLNECQRNAKIKSIRDD | 15 | Amide | 4078.6 | 4078.7 |
| PEP-06 | Free | FNMQQQRRFYEALHDPNLNEEQRCAKIKSIRDD | 16 | Amide | 4093.6 | 4093.7 |
| PEP-07 | Free | FNMQQQRRFYEALHDPNLNEEQRNAKIKSICDD | 17 | Amide | 4051.5 | 4051.6 |
| PEP-08 | Free | FNMQQQRRFYEALHDPNLNEEQRNAKIKSIRCD | 18 | Amide | 4092.6 | 4092.7 |
| PEP-09 | Free | FNMQQQRRFYEALHDPNLNHcyEQRNAKIKSIRDD | 19 | Amide | 4092.4 | 4092.8 |
| PEP-10 | 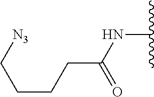 [SMILES: *NC(=O)CCCCN=[N+]=[N-]] | FNMQQQRRFYEALHDPNLNHcyEQRNAKIKSIRDD | 19 | Amide | 4217.5 | 4218.4 |
| PEP-11 | 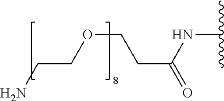 | FNMQQQRRFYEALHDPNLNHcyEQRNAKIKSIRDD | 19 | Amide | 4516.0 | 4516.1 |
| PEP-12 | Free | FNMQQQRRFYEALHDpNLNCEQRNAKIKSIRDD | 20 | Amide | 4078.6 | 4078.6 |
| PEP-13 | Free | FNMQQQRRFYEALHDPNLNEEQRNAKIKSIRHcyD | 21 | Amide | 4106.6 | 4106.7 |
| PEP-14 | Free | FNHcyQQQRRFYEALHDPNLNEEQRNAKIKSIRDD | 22 | Amide | 4090.5 | 4090.9 |

^{a}average molecular mass,
^{b}after deconvolution

### Example 4. Synthesis of Small Molecule Reagents

Synthesis of Electrophiles with Allyl Halides

### Synthesis of AHL-01

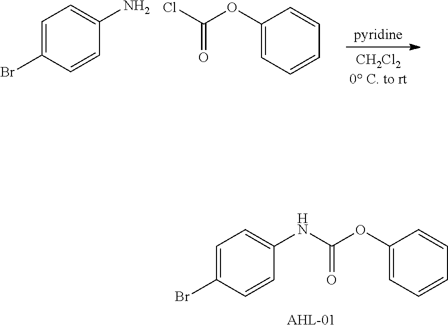

A 50 mL round-bottom flask equipped with a stir bar was charged with 4-bromoaniline (1000 mg, 5.81 mmol) followed by the addition of 20 mL of dichloromethane to provide a clear solution. The flask was cooled in an ice bath, and then phenyl chloroformate (803 μL, 6.40 mmol) and pyridine (562 μL, 6.98 mmol) were added, and the resulting mixture was stirred at room temperature for 2 hours. The resulting mixture was quenched with NaHCO₃ (100 mL) and partitioned between ethyl acetate (300 mL) and water (200 mL). The organic phase was collected, and the aqueous phase was extracted with ethyl acetate (100 mL) twice. The combined organic extract was washed with 1M HCl aq. (100 mL) and brine (50 mL), dried over magnesium sulfate, filtered, and concentrated under reduced pressure. 500 mg of the 1.61 g residue obtained was then treated with a mixture of hexane and diethyl ether (10:1, 15 mL) and subjected to sonication for approximately 30 seconds. The reaction solution was filtered using a Büchner funnel, and the resulting solid was washed with hexane and diethyl ether (10:1, 5 mL). Finally, the solid was dried under reduced pressure, yielding the desired compound as a white solid (460 mg, 87%). The 1H and mass spectra of the obtained material were identical to those reported in the patent (WO2017205709).

### Synthesis of AHL-02

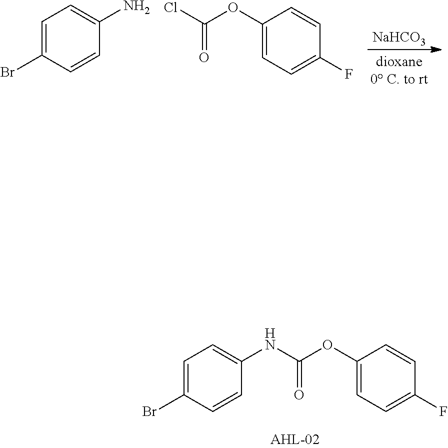

A 50 mL round-bottom flask equipped with a stir bar was charged with 4-bromoaniline (250 mg, 1.45 mmol) followed by the addition of 4 mL of dioxane to provide a clear solution. The flask was cooled in an ice bath, and then 4-fluorophenyl chloroformate (279 mg, 1.60 mmol) and NaHCO3 (244 mg, 2.91 mmol) were added, and the resulting mixture was stirred at room temperature for 2 hours. The resulting mixture was quenched with water (100 mL) and partitioned between ethyl acetate (300 mL) and water (200 mL). The organic phase was collected, and the aqueous phase was extracted with ethyl acetate (100 mL) twice. The combined organic extract was washed with brine (50 mL), dried over magnesium sulfate, filtered, and concentrated under reduced pressure. The obtained residue was then treated with a mixture of hexane and diethyl ether (10:1, 15 mL) and subjected to sonication for approximately 30 seconds. The reaction solution was filtered using a Büchner funnel, and the resulting solid was washed with a solution of hexane and diethyl ether (10:1, 5 mL). Finally, the solid was dried under reduced pressure, yielding the desired compound as a white solid (183 mg, 41%). ¹H NMR (400 MHZ, CDCl3): δ 7.46 (d, J=8.8 Hz, 2H), 7.34 (d, J=8.4 Hz, 2H), 7.15 (dd, J=9.1, 4.5 Hz, 2H), 7.08 (dd, J=9.1, 8.0 Hz, 2H), 6.90 (brs, ¹H).

### Synthesis of AHL-03

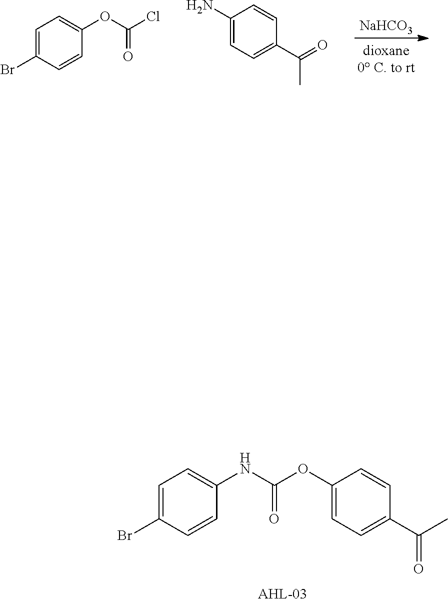

A 50 mL round-bottom flask equipped with a stir bar was charged with 4′-Aminoacetophenone (110 mg, 0.815 mmol), followed by the addition of 4 mL of dioxane to provide a clear solution. The flask was cooled in an ice bath, and then 4-bromophenyl chloroformate (230 mg, 0.978 mmol) and NaHCO3 (137 mg, 1.63 mmol) were added, and the resulting mixture was stirred at room temperature for 2 hours. The resulting mixture was quenched with water (100 mL) and partitioned between ethyl acetate (200 mL) and water (100 mL). The organic phase was collected, and the aqueous phase was extracted with ethyl acetate (100 mL) twice. The combined organic extract was washed with brine (50 mL), dried over magnesium sulfate, filtered, and concentrated under reduced pressure. The obtained residue was then treated with a mixture of hexane and diethyl ether (10:1, 15 mL) and subjected to sonication for approximately 30 seconds. The reaction solution was filtered using a Büchner funnel, and the resulting solid was washed with a solution of hexane and diethyl ether (10:1, 5 mL). Finally, the solid was dried under reduced pressure, yielding the desired compound as a white solid (183 mg, 67%).

1H NMR (400 MHZ, DMSO): δ 10.68 (s, 1H), 7.95 (d, J=8.8 Hz, 2H), 7.69-7.59 (m, 4H), 7.25 (d, J=8.8 Hz, 2H), 2.53 (s, 3H).

### Synthesis of AHL-04

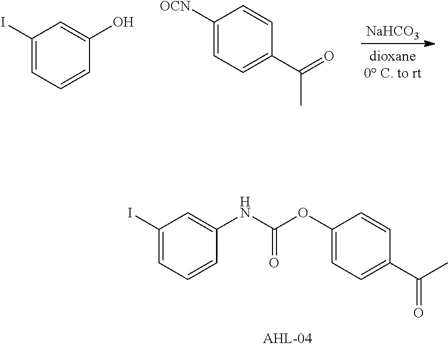

A 50 mL round-bottom flask equipped with a stir bar was charged with 3-iodophenol (324 mg, 1.47 mmol), followed by the addition of 7 mL of CH2Cl2 to provide a clear solution. Next, 4-Acetylphenyl Isocyanate (250 mg, 1.55 mmol) and DIEA (330 μL, 1.86 mmol) were added, and the resulting mixture was stirred at room temperature for 1 hour. The resulting mixture was then treated with diethyl ether (4 mL) and subjected to sonication for approximately seconds. The reaction solution was filtered using a Büchner funnel, and the resulting solid was washed with dichloromethane (1 mL). Finally, the solid was dried under reduced pressure, yielding the desired compound as a white solid (241 mg, 43%).

1H NMR (400 MHZ, DMSO): δ 10.68 (s, 1H), 7.95 (d, J=8.8 Hz, 2H), 7.72-7.59 (m, 4H), 7.34-7.20 (m, 2H), 2.53 (s, 3H).

### Synthesis of AHL-05

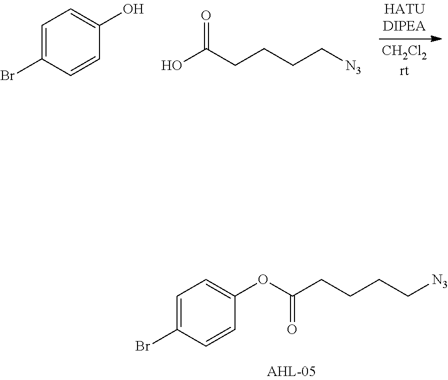 [SMILES: Oc1ccc(Br)cc1 ; [N-]=[N+]=NCCCCC(=O)O ; [N-]=[N+]=NCCCCC(=O)Oc1ccc(Br)cc1]

A 100 mL round-bottom flask equipped with a stir bar was charged with 4-bromophenol (314 mg, 1.82 mmol), followed by the addition of 8 mL of dioxane to provide a clear solution. Next, 5-azidopentanoic acid (200 mg, 1.40 mmol), DIEA (0.973 μL, 5.59 mmol), and HATU (797 mg, 2.10 mmol) were added, and the resulting mixture was stirred at room temperature for hours. The resulting mixture was quenched with saturated NaHCO3 solution (100 mL) and partitioned between CH2Cl2 (200 mL) and water (100 mL). The organic phase was collected, and the aqueous phase was extracted with CH2Cl2 (100 mL) twice. The combined organic extract was washed with brine (50 mL), dried over sodium sulfate, filtered, and concentrated under reduced pressure. The obtained residue was purified by column chromatography (using Biotage® Sfär Silica 60 μm 25 g column, 95:5 to 3:1 hexane:ethyl acetate) and dried under reduced pressure, yielding the desired compound as a clear oil (338 mg, 81%).

1H NMR (400 MHZ, CDCl3): δ 7.49 (d, J=8.8 Hz, 2H), 6.97 (d, J=8.8 Hz, 2H), 3.35 (t, J=6.6 Hz, 2H), 2.60 (t, J=7.3 Hz, 2H), 1.90-1.78 (m, 2H), 1.77-1.67 (m, 2H).

### Synthesis of AHL-06

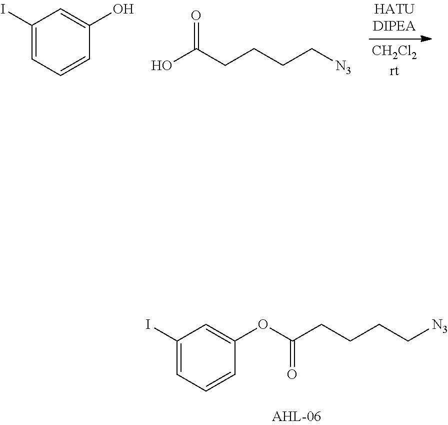 [SMILES: Oc1cccc(I)c1 ; [N-]=[N+]=NCCCCC(=O)O ; [N-]=[N+]=NCCCCC(=O)Oc1cccc(I)c1]

A 100 mL round-bottom flask equipped with a stir bar was charged with 3-iodophenol (400 mg, 1.82 mmol), followed by the addition of 8 mL of CH2Cl2 to provide a clear solution. Next, 5-azidopentanoic acid (200 mg, 1.40 mmol), DIEA (0.973 μL, 5.59 mmol), and HATU (797 mg, 2.10 mmol) were added, and the resulting mixture was stirred at room temperature for hours. The resulting mixture was quenched with saturated NaHCO3 solution (100 mL) and partitioned between CH2Cl2 (200 mL) and water (100 mL). The organic phase was collected, and the aqueous phase was extracted with CH2Cl2 (100 mL) twice. The combined organic extract was washed with brine (50 mL), dried over sodium sulfate, filtered, and concentrated under reduced pressure. The obtained residue was purified by column chromatography (using Biotage® Sfär Silica 60 μm 25 g column, 95:5 to 3:1 hexane:ethyl acetate) and dried under reduced pressure, yielding the desired compound as a clear oil (269 mg, 78%).

1H NMR (400 MHZ, CDCl3): δ 7.57 (dt, J=7.5, 1.5 Hz, 1H), 7.46 (t, J=1.8 Hz, 1H), 7.15-7.03 (m, 2H), 3.35 (t, J=6.6 Hz, 2H), 2.60 (t, J=7.3 Hz, 2H), 1.90-1.78 (m, 2H), 1.79-1.66 (m, 2H).

### Synthesis of AHL-07

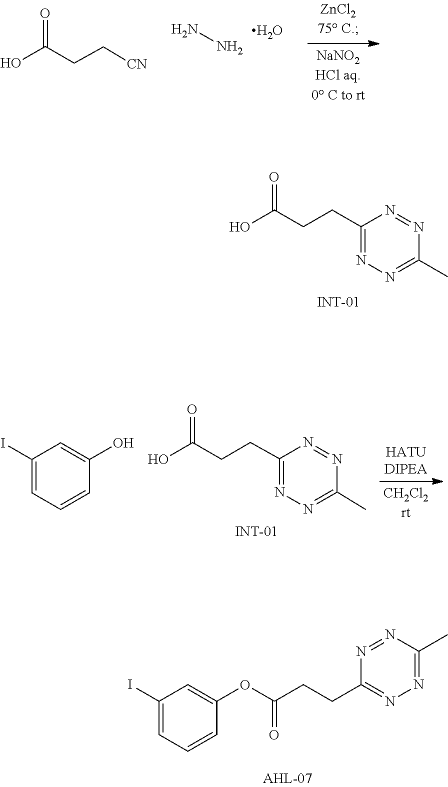

The INT-01 was synthesized as previously described (WO2014065860). The 1H NMR spectra of the obtained material were identical to those reported in the literature.

A 20 mL scintillation glass vial equipped with a stir bar was charged with 3-iodophenol (45.4 mg, 0.309 mmol), followed by the addition of 2 mL of CH2Cl2 to provide a clear solution. Next, INT-01 (40.0 mg, 0.238 mmol), DIEA (104 μL, 0.952 mmol), and HATU (90.5 mg, 0.357 mmol) were added, and the resulting mixture was stirred at room temperature for 12 hours. The resulting mixture was directly purified by column chromatography (using Biotage® Sfär Silica 60 μm 10 g column, 80:20 to 1:1 hexane:ethyl acetate) and dried under reduced pressure, yielding the desired compound as a pink oil (50 mg, 57%).

1H NMR (400 MHZ, CDCl3): δ 7.57 (ddd, J=6.0, 3.1, 1.6 Hz, 1H), 7.50-7.44 (m, 1H), 7.14-7.05 (m, 2H), 3.75 (t, J=6.9 Hz, 2H), 3.31 (t, J=6.9 Hz, 2H), 3.06 (s, 3H).

### Synthesis of AHL-08

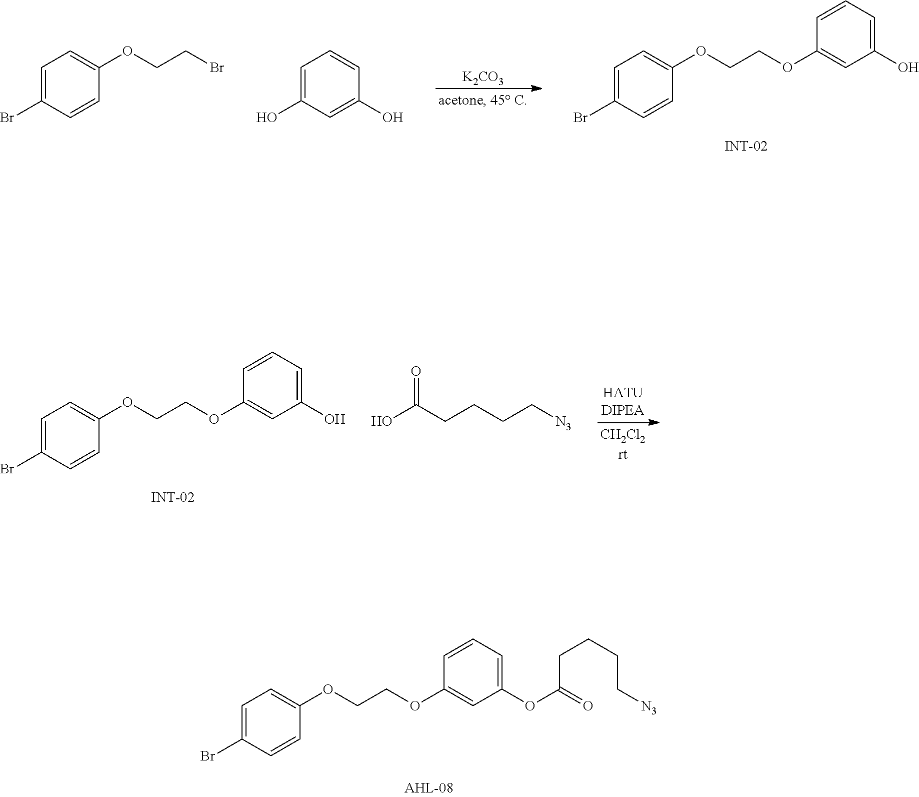

A 50 mL round-bottom flask equipped with a stir bar was charged with 1-bromo-4-(2-bromoethoxy)benzene (600 mg, 2.14 mmol), followed by the addition of 10 mL of acetone to provide a clear solution. Next, resorcinol (1.18 g, 10.7 mmol) and K2CO3 (1.48 g, 10.7 mmol) were added, and the resulting mixture was stirred at 45° C. for 12 hours. The resulting mixture was cooled to room temperature, followed by filtration using a Büchner funnel. The collected solid was then washed once with acetone (5 mL), and the resulting solution was concentrated under reduced pressure. The obtained residue was purified by column chromatography (using Biotage® Sfär Silica 60 μm 25 g column, 95:5 to 3:1 hexane:ethyl acetate) and dried under reduced pressure, yielding the desired compound as a clear oil (415 mg, 63%).

1H NMR (400 MHz, CDCl3): δ 7.39 (d, J=9.0 Hz, 2H), 7.14 (t, J=8.5 Hz, 1H), 6.83 (d, J=9.0 Hz, 2H), 6.57-6.49 (m, 1H), 6.47-6.43 (m, 2H), 4.71 (s, 1H), 4.28 (s, 4H).

A 20 mL scintillation glass vial equipped with a stir bar was charged with INT-02 (150 mg, 0.485 mmol), followed by the addition of 2 mL of CH2Cl2 to provide a clear solution. Next, 5-azidopentanoic acid (63.1 mg, 0.441 mmol), DIEA (300 μL, 1.76 mmol), and HATU (252 mg, 0.662 mmol) were added, and the resulting mixture was stirred at room temperature for 12 hours. The resulting mixture was directly purified by column chromatography (using Biotage® Sfär Silica 60 μm 10 g column, 80:20 to 1:1 hexane:ethyl acetate) and dried under reduced pressure, yielding the desired compound as a pale pink solid (116 mg, 61%).

1H NMR (400 MHZ, CDCl3): δ 7.39 (d, J=9.0 Hz, 2H), 7.31-7.26 (m, 1H), 6.87-6.78 (m, 3H), 6.74-6.66 (m, 2H), 4.29 (s, 4H), 3.35 (t, J=6.7 Hz, 2H), 2.60 (t, J=7.2 Hz, 2H), 1.91-1.79 (m, 2H), 1.78-1.67 (m, 2H).

### Synthesis of AHL-09

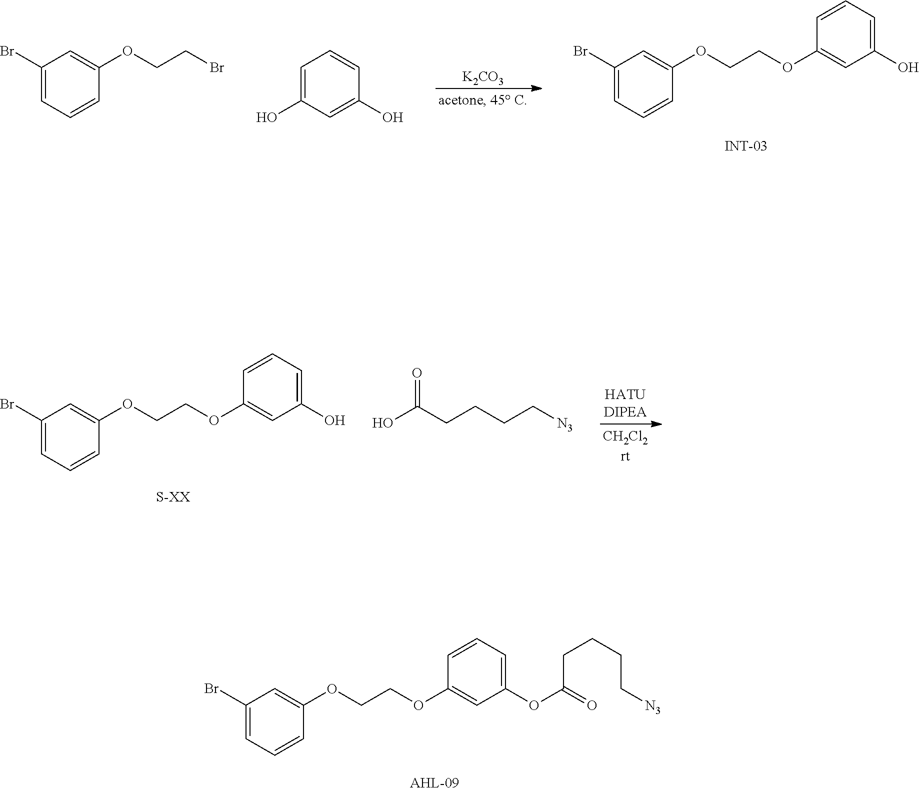

A 50 mL round-bottom flask equipped with a stir bar was charged with 1-bromo-3-(2-bromoethoxy)benzene (600 mg, 2.14 mmol), followed by the addition of 10 mL of acetone to provide a clear solution. Next, resorcinol (1.18 g, 10.7 mmol) and K2CO3 (1.48 g, 10.7 mmol) were added, and the resulting mixture was stirred at 45° C. for 12 hours. The resulting mixture was cooled to room temperature, followed by filtration using a Büchner funnel. The collected solid was then washed once with acetone (5 mL), and the resulting solution was concentrated under reduced pressure. The obtained residue was purified by column chromatography (using Biotage® Sfär Silica 60 μm 25 g column, 95:5 to 3:1 hexane:ethyl acetate) and dried under reduced pressure, yielding the desired compound as a clear oil (415 mg, 63%).

1H NMR (400 MHZ, CDCl3): δ 7.20-7.07 (m, 4H), 6.91-6.86 (m, 1H), 6.53 (ddd, J=8.3, 2.3, 1.0 Hz, 1H), 6.48-6.42 (m, 2H), 4.72 (s, 1H), 4.29 (s, 4H).

A 20 mL scintillation glass vial equipped with a stir bar was charged with INT-03 (75 mg, 0.242 mmol), followed by the addition of 2 mL of CH2Cl2 to provide a clear solution. Next, 5-azidopentanoic acid (52.0 mg, 0.363 mmol), DIEA (158 μL, 0.968 mmol), and HATU (184 mg, 0.484 mmol) were added, and the resulting mixture was stirred at room temperature for 12 hours. The resulting mixture was directly purified by column chromatography (using Biotage® Sfär Silica 60 μm 10 g column, 80:20 to 1:1 hexane:ethyl acetate) and dried under reduced pressure, yielding the desired compound as a pale pink solid (74 mg, 70%).

1H NMR (400 MHZ, CDCl3): δ 7.29 (t, J=8.1 Hz, 1H), 7.20-7.09 (m, 3H), 6.92-6.79 (m, 2H), 6.75-6.66 (m, 2H), 3.35 (t, J=6.7 Hz, 2H), 2.60 (t, J=7.2 Hz, 2H), 1.91-1.79 (m, 2H), 1.78-1.68 (m, 2H).

### Synthesis of AHL-10

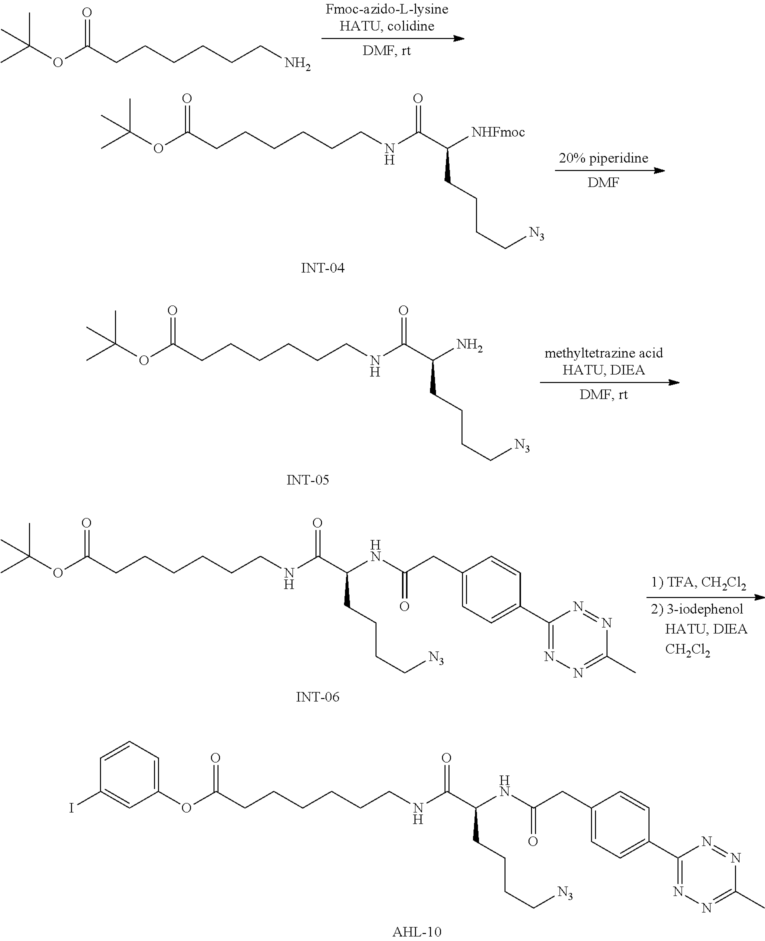

A 50 mL round-bottom flask equipped with a stir bar was charged with 7-amino-heptanoic acid t-butyl ester (346 mg, 1.72 mmol), followed by the addition of 9 mL of DMF to provide a clear solution. Next, Fmoc-L-azidolysine (746 mg, 1.89 mmol), 2,4,6-colidine (0.909 μL, 6.84 mmol), and HATU (781 mg, 2.06 mmol) were added, and the resulting mixture was stirred at room temperature for 12 hours. The resulting mixture was quenched with saturated NaHCO₃ solution (100 mL) and partitioned between ethyl acetate (200 mL) and water (100 mL). The organic phase was collected, and the aqueous phase was extracted with ethyl acetate (100 mL) twice. The combined organic extract was washed with saturated ammonium chloride solution (100 mL) twice and brine (50 mL), dried over sodium sulfate, filtered, and concentrated under reduced pressure. The obtained residue was purified by column chromatography (using Biotage® Sfär Silica 60 μm 25 g column, 95:5 to 2:1 hexane:ethyl acetate) and dried under reduced pressure, yielding INT-04 as a clear oil (868 mg, 87%).

1H NMR (400 MHZ, CDCl3): δ 7.77 (d, J=7.5 Hz, 2H), 7.58 (d, J=7.5 Hz, 2H), 7.41 (t, J=7.5 Hz, 2H), 7.32 (t, J=7.4 Hz, 2H), 5.90 (s, 1H), 5.32 (d, J=8.3 Hz, 1H), 4.42 (s, 2H), 4.21 (t, J=6.7 Hz, 1H), 3.34-3.17 (m, 4H), 2.19 (t, J=7.4 Hz, 2H), 1.57 (s, 8H), 1.43 (s, 9H), 1.43-1.40 (m, 2H), 1.31 (s, 4H).

A 50 mL round-bottom flask equipped with a stir bar was charged with INT-04 (400 mg, 0.69 mmol), followed by the addition of piperidine in DMF (3.4 mL, 20 v/v %) to provide a clear solution. The resulting mixture was stirred at room temperature for 2 hours. The resulting mixture was diluted with 20 mL of toluene and concentrated under reduced pressure. To remove DMF, 20 mL of toluene was added to the reaction mixture, and the solvent was subsequently evaporated under reduced pressure three times. The obtained residue was purified by column chromatography (using Biotage® Sfär Silica 60 μm 25 g column, 100:0 to 95:5 CH₂Cl2+0.5 v/v % trimethylamine: MeOH) and dried under reduced pressure, yielding INT-05 as a clear oil (162 mg, 66%).

1H NMR (400 MHZ, CDCl3): δ 7.30 (s, 1H), 3.43 (dd, J=7.7, 4.8 Hz, 1H), 3.34-3.19 (m, 4H), 2.20 (t, J=7.5 Hz, 2H), 2.11-1.69 (m, 4H), 1.69-1.44 (m, 8H), 1.44 (s, 9H), 1.33 (p, J=3.5 Hz, 4H).

A 20 mL scintillation glass vial equipped with a stir bar was charged with INT-05 (93 mg, 0.261 mmol), followed by the addition of 2 mL of CH2Cl2 to provide a clear solution. Next, Methyltetrazine acid (50 mg, 0.217 mmol), DIEA (154 μL, 0.868 mmol), and HATU (124 mg, 0.326 mmol) were added, and the resulting mixture was stirred at room temperature for 12 hours. The resulting mixture was directly purified by column chromatography (using Biotage® Sfär Silica 60 μm 10 g column, 80:20 to 1:1 hexane:ethyl acetate) and dried under reduced pressure, yielding INT-06 as a pale pink solid (101 mg, 82%).

1H NMR (400 MHZ, CDCl3): δ 8.58 (d, J=8.4 Hz, 2H), 7.50 (d, J=8.2 Hz, 2H), 6.19 (d, J=8.0 Hz, 1H), 5.98 (s, 1H), 4.36 (q, J=7.2 Hz, 1H), 3.68 (s, 2H), 3.23 (dq, J=7.3, 4.9 Hz, 4H), 3.10 (s, 3H), 2.19 (t, J=7.4 Hz, 2H), 1.90-1.77 (m, 1H), 1.66-1.41 (m, 7H), 1.44 (s, 9H), 1.40-1.24 (m, 6H).

A 10 mL round-bottom flask equipped with a stir bar was charged with INT-06 (30 mg, 0.053 mmol), followed by the addition of dichloromethane (2 mL) to provide a clear solution. Next, TFA (0.4 mL) was added, and the resulting mixture was stirred at room temperature for 3 hours. The reaction mixture was concentrated under reduced pressure, and the resulting residue was used in the next step without further purification.

Dichloromethane (1 mL) was added to a 10 mL round-bottom flask containing the residue obtained from the previous reaction, resulting in a clear solution. Next, 3-iodophenol (17.5 mg, 0.080 mmol), DIEA (55.0 μL, 0.318 mmol), and HATU (34.3 mg, 0.090 mmol) were added, and the resulting mixture was stirred at room temperature for 12 hours. The resulting mixture was directly purified by column chromatography (using Biotage® Sfär Silica 60 μm 10 g column, 80:20 to 1:1 hexane:ethyl acetate) and dried under reduced pressure, yielding AHL-10 as a pale pink solid (35.9 mg, 95%).

MS (m/z): C30H37IN9O4+ (M+H+) 714.2, found 714.0′

### Synthesis of Palladium Oxidative Addition Complexes

Synthesis of [(cod)Pd(CH2TMS)2] (cod=1,5 cyclooctadiene)

This compound was synthesized as previously described (Nature 526, 687-691 (2015)). The ¹H and ¹³C NMR spectra of the obtained material were identical to those reported in the literature.

Synthesis of Sodium 2′-dicyclohexylphosphino-2,6-dimethoxy-1,1′-biphenyl-3-sulfonate hydrate (sSPhos)

This compound was synthesized as previously described (Org. Lett. 23, 777-780 (2021)). The ¹H and ¹³C NMR spectra of the obtained material were identical to those reported in the literature.

### Synthesis of OAC-01 (General Procedure)

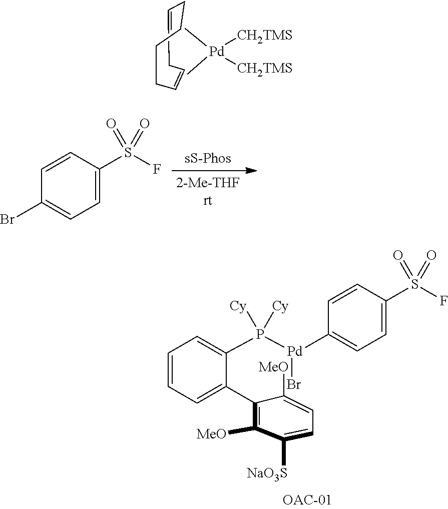

A 5 mL scintillation glass vial equipped with a stir bar was charged with 4-bromobenzenesulfonyl fluoride (20.4 mg, 0.085 mmol) and sSPhos (43.4 mg, 0.085 mmol), and 2-methyl tetrahydrofuran (0.51 mL). The reaction solution was sonicated for 1 minute until it became completely clear, and then the [(cod)Pd(CH2TMS)2] (30.0 mg, 0.077 mmol) was added, and the resulting mixture was stirred at room temperature for 2 hours. After the reaction time, pentane (1 mL) was added to the reaction mixture. The vial containing the white suspension was placed in a centrifuge and centrifuged at 3220 ref for 2 minutes. The cap was removed, and the supernatant was decanted. The stir bar was removed, and the solid was resuspended in 2-methyltetrahydrofuran (1.0 mL) and pentane (1.0 mL). The vial was capped and sonicated for 1 minute to obtain a homogeneous suspension. The vial was then centrifuged again at 3220 ref for 2 minutes. The cap was removed, and the supernatant was decanted. This sonication/centrifugation/decanting procedure was repeated twice. The resulting beige solid was dried under reduced pressure, yielding the desired compound as a pale brown solid (51 mg, 77%).

MS (m/z): C32H39FO7PPdS2+ (M−Br−Na+H+) 755.1, found 755.0.

### Synthesis of OAC-02

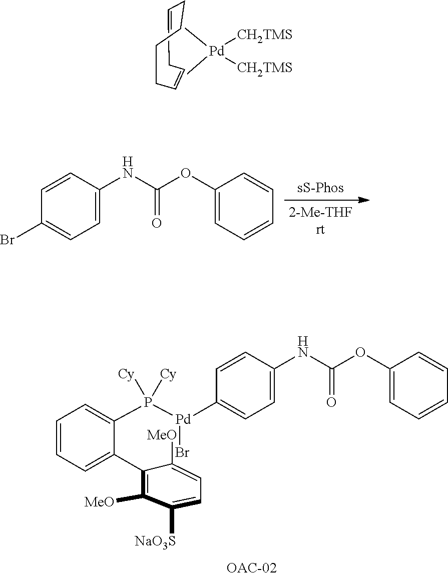

This compound was synthesized following the general procedure described in the section on the synthesis of OAC-01. AHL-01 (16.6 mg, 0.057 mmol), sSPhos (28.9 mg, 0.056 mmol), [(cod)Pd(CH2TMS)2] (20.0 mg, 0.052 mmol), and 2-methyltetrahydrofuran (0.34 mL) were used, and the desired compound was obtained as a light brown solid (11 mg, 25%).

MS (m/z): C39H45NO7PPdS+ (M−Br−Na+H+) 808.2, found 808.1.

### Synthesis of OAC-03

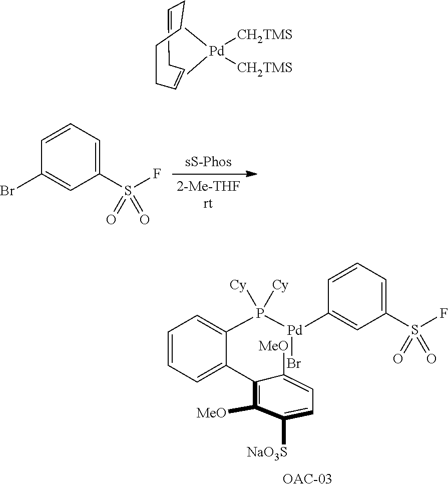

This compound was synthesized following the general procedure described in the section on the synthesis of OAC-01. 3-bromobenzenesulfonyl fluoride (6.8 mg, 0.029 mmol), sSPhos (14.5 mg, 0.028 mmol), [(cod)Pd(CH2TMS)2] (10.0 mg, 0.026 mmol), and 2-methyltetrahydrofuran (0.34 mL) were used, and OAC-03 was obtained as a light brown solid (21 mg, 95%).

MS (m/z): C32H39FO7PPdS2+ (M−Br−Na+H+) 755.1, found 755.0.

### Synthesis of OAC-04

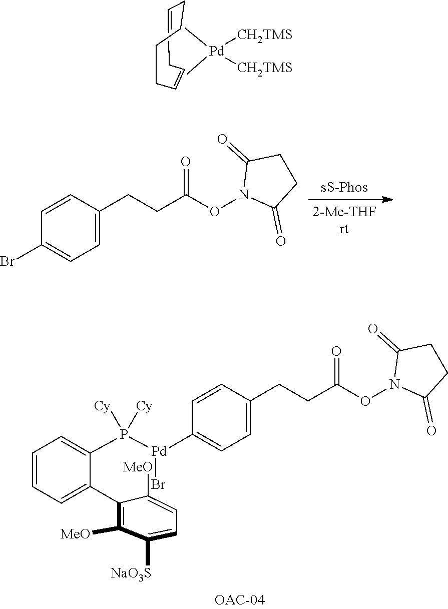

This compound was synthesized as previously described (J. Am. Chem. Soc., 142, 21237-21242 (2020)). The ¹H, ¹³C and ³¹P NMR spectra of the obtained material were identical to those reported in the literature.

### Synthesis of OAC-05

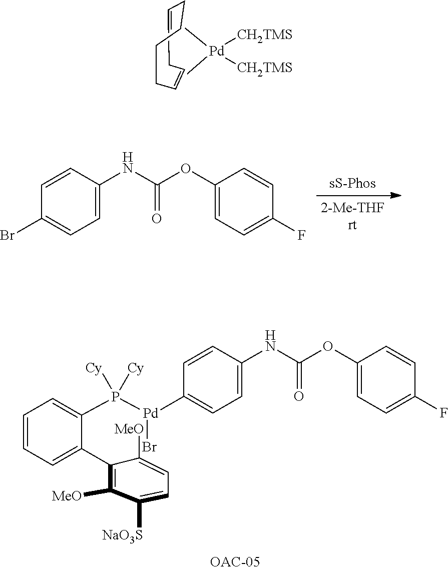

This compound was synthesized following the general procedure described in the section on the synthesis of OAC-01. AHL-02 (17.7 mg, 0.057 mmol), sSPhos (28.9 mg, 0.056 mmol), [(cod)Pd(CH2TMS)2] (20.0 mg, 0.052 mmol), and 2-methyltetrahydrofuran (0.5 mL) were used, and the desired compound was obtained as a light brown solid (41 mg, 87%).

MS (m/z): C39H44FNO7PPdS+ (M−Br−Na+H+) 826.2, found 826.1.

### Synthesis of OAC-06

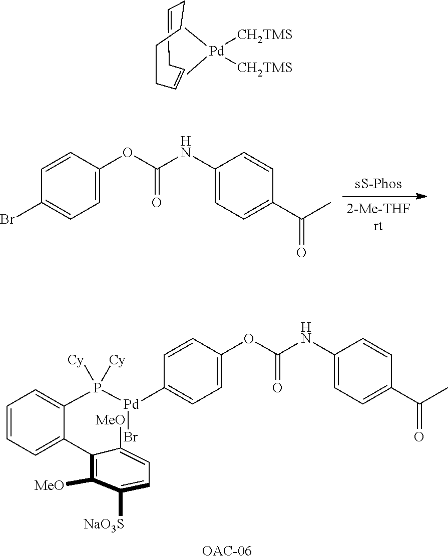

This compound was synthesized following the general procedure described in the section on the synthesis of OAC-01. AHL-03 (9.5 mg, 0.028 mmol), sSPhos (14.5 mg, 0.028 mmol), [(cod)Pd(CH2TMS)2] (10.0 mg, 0.026 mmol), and 2-methyltetrahydrofuran (0.5 mL) were used, and OAC-06 was obtained as a light brown solid (12 mg, 50%).

MS (m/z): C41H47NO8PPdS+ (M−Br−Na+H+) 850.2, found 850.1.

### Synthesis of OAC-07

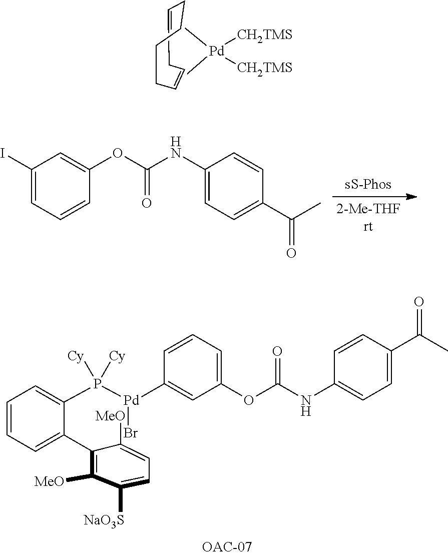

This compound was synthesized following the general procedure described in the section on the synthesis of OAC-01. AHL-04 (16.3 mg, 0.043 mmol), sSPhos (20.7 mg, 0.040 mmol), [(cod)Pd(CH2TMS)2] (15.0 mg, 0.039 mmol), and 2-methyltetrahydrofuran (0.5 mL) were used, and the desired compound was obtained as a light brown solid (39 mg, quantitative).

MS (m/z): C41H47NO8PPdS+ (M−I−Na+H+) 850.2, found 850.1.

### Synthesis of OAC-08

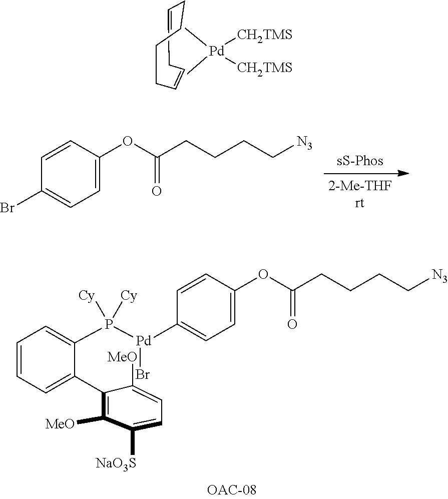

This compound was synthesized following the general procedure described in the section on the synthesis of OAC-01. AHL-05 (12.7 mg, 0.043 mmol), sSPhos (20.7 mg, 0.040 mmol), [(cod)Pd(CH2TMS)2] (15.0 mg, 0.039 mmol), and 2-methyltetrahydrofuran (0.5 mL) were used, and OAC-08 was obtained as a light brown solid (34 mg, 96%).

MS (m/z): C37H47NO7PPdS+ (M−N2−Br−Na+H+) 786.2, found 786.1.

### Synthesis of OAC-09

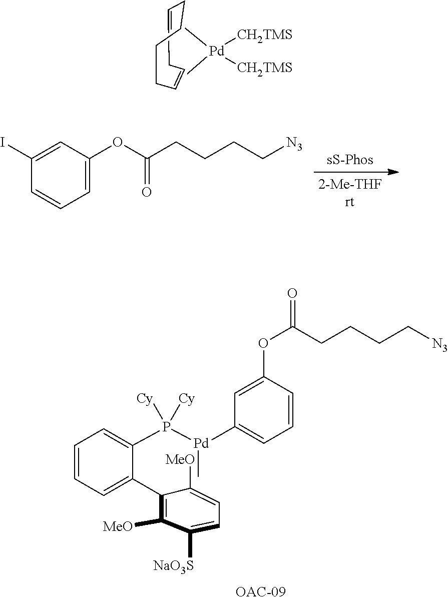

This compound was synthesized following the general procedure described in the section on the synthesis of OAC-01. ALH-06 (58.8 mg, 0.171 mmol), sSPhos (82.8 mg, 0.162 mmol), [(cod)Pd(CH2TMS)2] (60.0 mg, 0.154 mmol), and 2-methyltetrahydrofuran (1.0 mL) were used, and OAC-09 was obtained as a light brown solid (111 mg, 75%).

MS (m/z): C37H47N3O7PPdS+ (M−I−Na+H+) 814.2, found 814.1.

### Synthesis of OAC-10

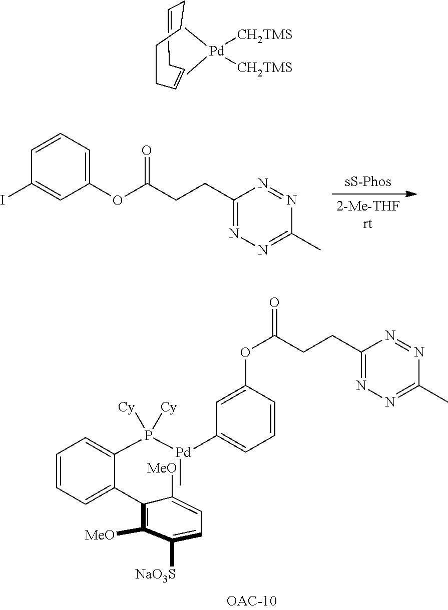

This compound was synthesized following the general procedure described in the section on the synthesis of OAC-01. ALH-07 (15.8 mg, 0.043 mmol), sSPhos (20.7 mg, 0.040 mmol), [(cod)Pd(CH2TMS)2] (15.0 mg, 0.039 mmol), and 2-methyltetrahydrofuran (0.5 mL) were used, and OAC-10 was obtained as a light brown solid (21 mg, 55%).

MS (m/z): C38H46N4O7PPdS+ (M−I−Na+H+) 839.2, found 839.1.

### Synthesis of OAC-11

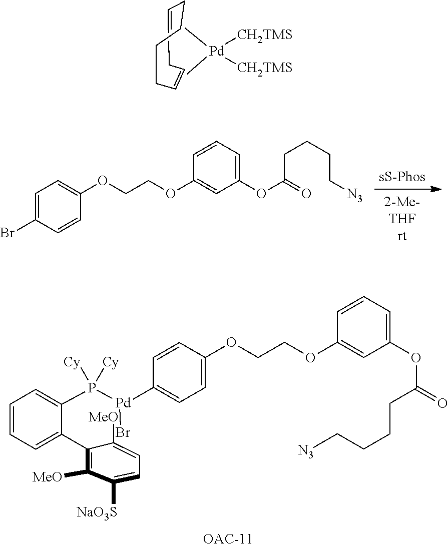

This compound was synthesized following the general procedure described previously. ALH-08 (18.5 mg, 0.043 mmol), sSPhos (20.7 mg, 0.040 mmol), [(cod)Pd(CH2TMS)2] (15.0 mg, 0.039 mmol), and 2-methyltetrahydrofuran (0.5 mL) were used, and OAC-11 was obtained as a light brown solid (32 mg, 80%).

MS (m/z): C45H55N3O9PPdS+ (M−Br−Na+H+) 950.0, found 950.1.

### Synthesis of OAC-12

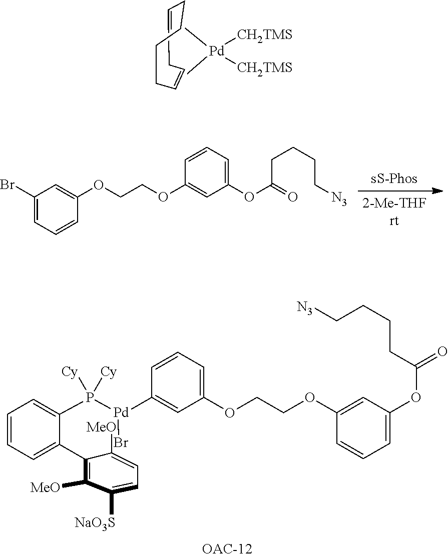

This compound was synthesized following the general procedure described previously. ALH-09 (17.0 mg, 0.039 mmol), sSPhos (19.0 mg, 0.037 mmol), [(cod)Pd(CH2TMS)2] (13.8 mg, 0.035 mmol), and 2-methyltetrahydrofuran (0.5 mL) were used, and OAC-12 was obtained as a light brown solid (26 mg, 71%).

MS (m/z): C45H55N3O9PPdS+ (M−Br−Na+H+) 950.0, found 950.1.

### Synthesis of OAC-13

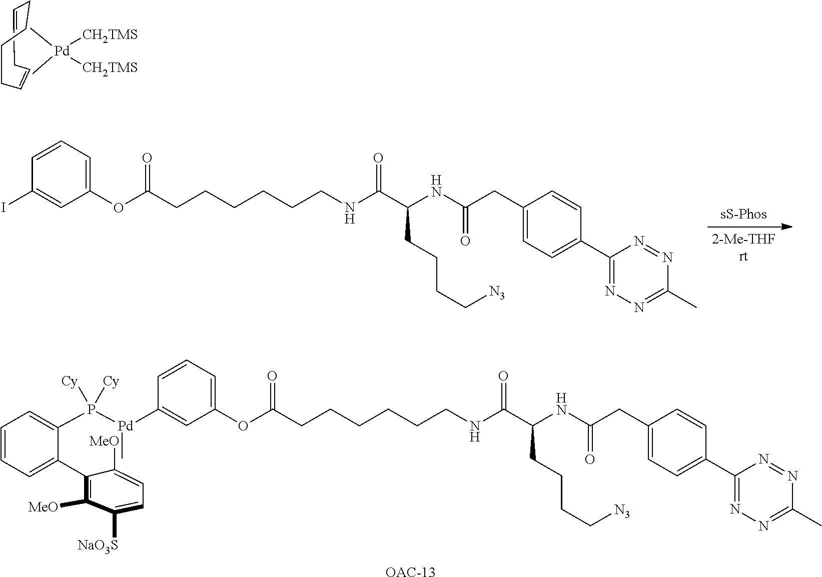

This compound was synthesized following the general procedure described previously ALH-10 (15.0 mg, 0.021 mmol), sSPhos (10.3 mg, 0.020 mmol), [(cod)Pd(CH2TMS)2] (7.5 mg, 0.019 mmol), and 2-methyltetrahydrofuran (0.4 mL) were used, and OAC-12 was obtained as a light brown solid (22 mg, 85%).

MS (m/z): C56H71N9O9PPdS+ (M−I−Na+H+) 1182.0, found 1182.0.

### Example 5. Synthesis of Electrophile-Attached Reagents

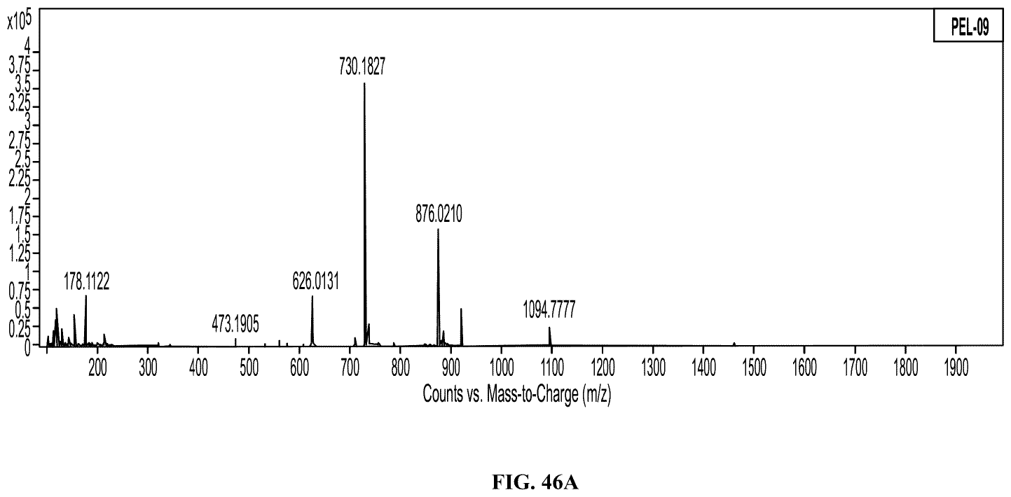

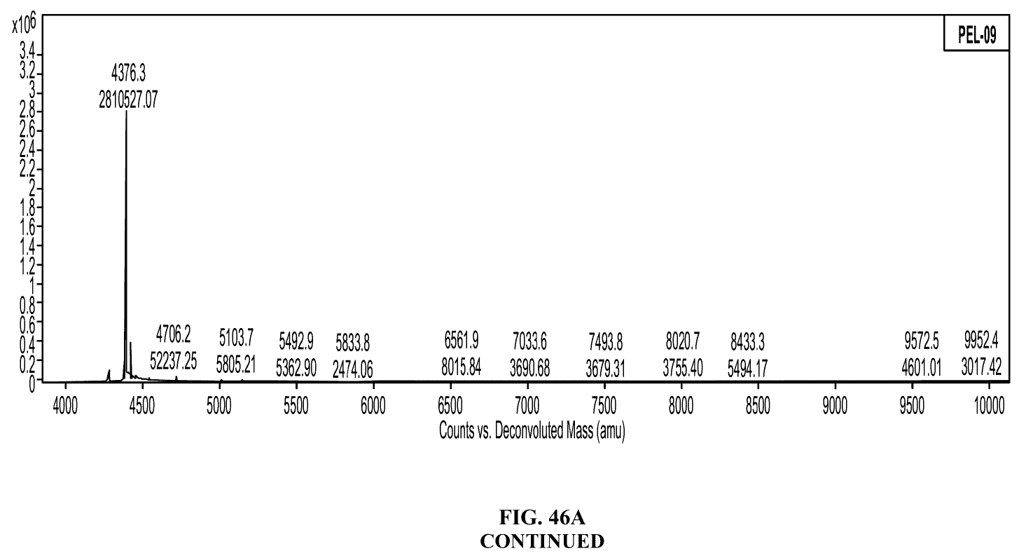

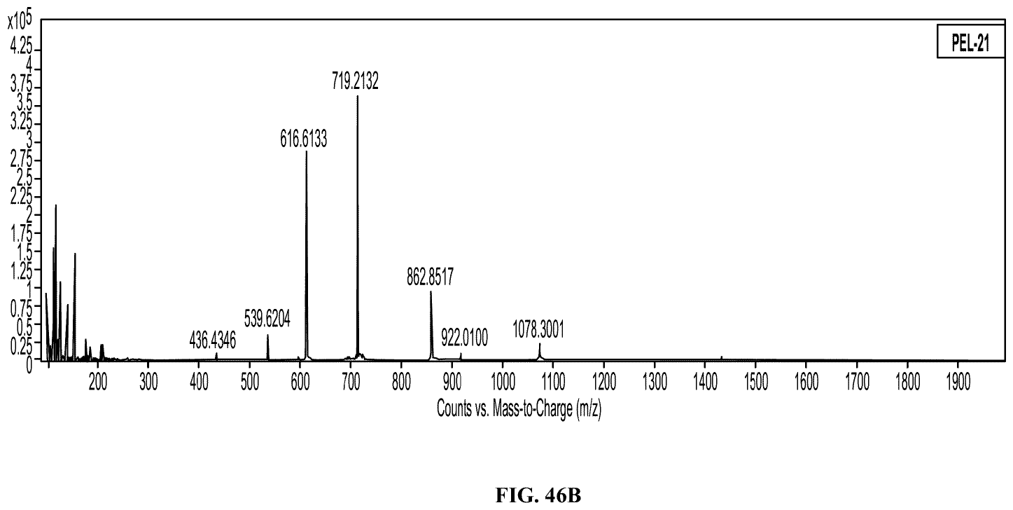

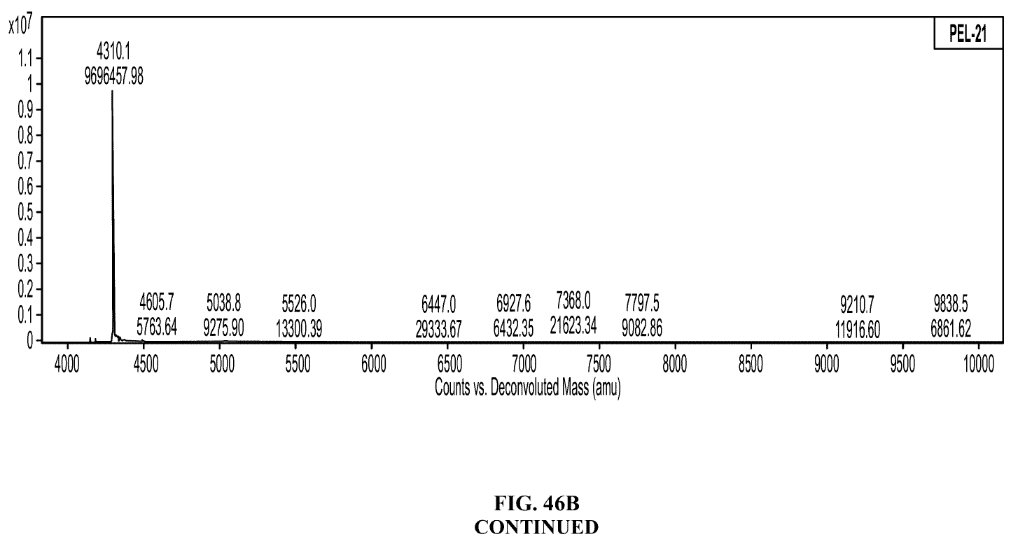

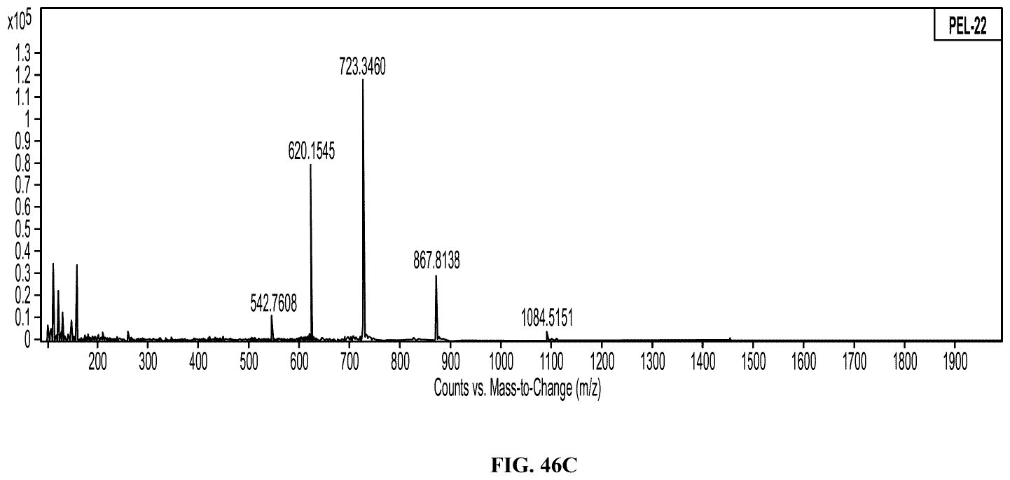

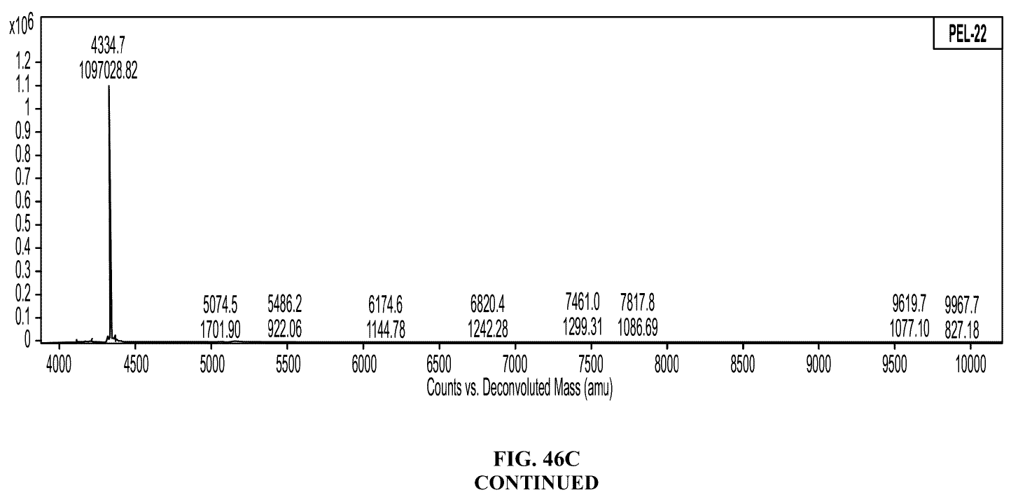

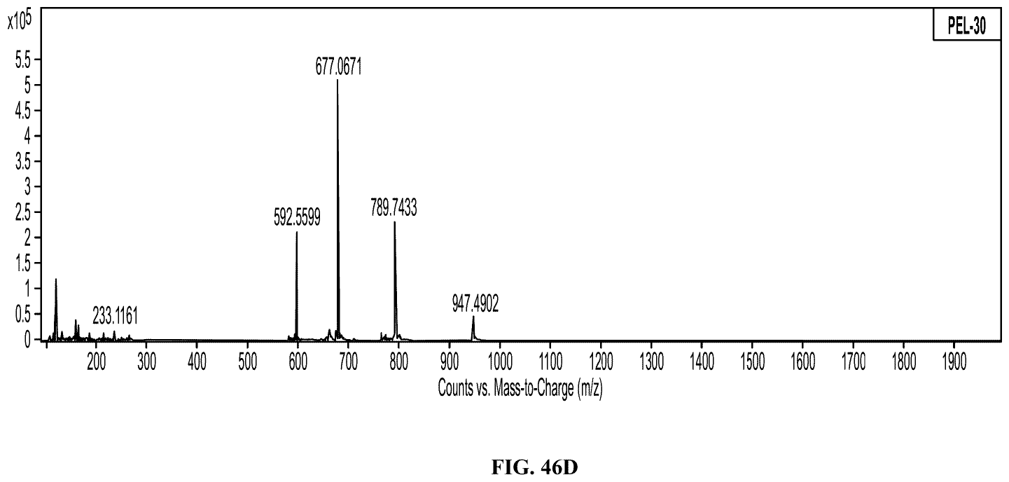

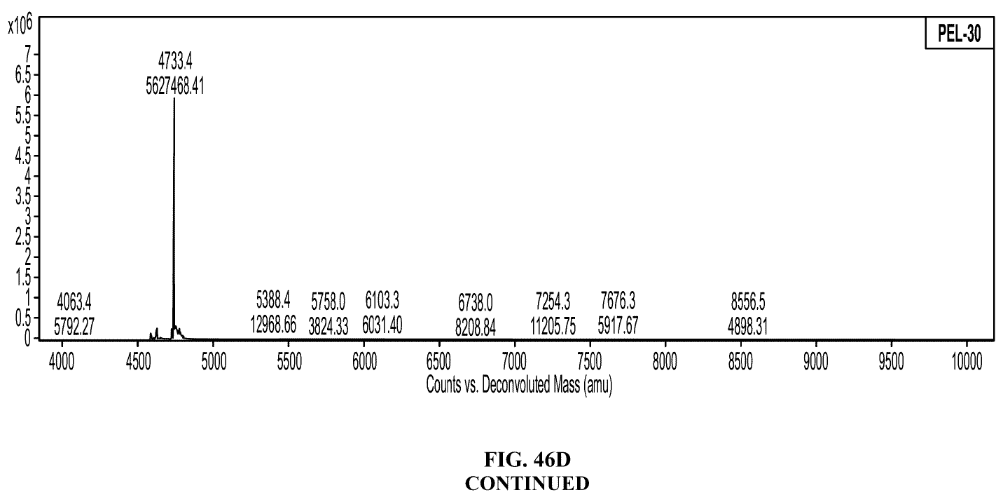

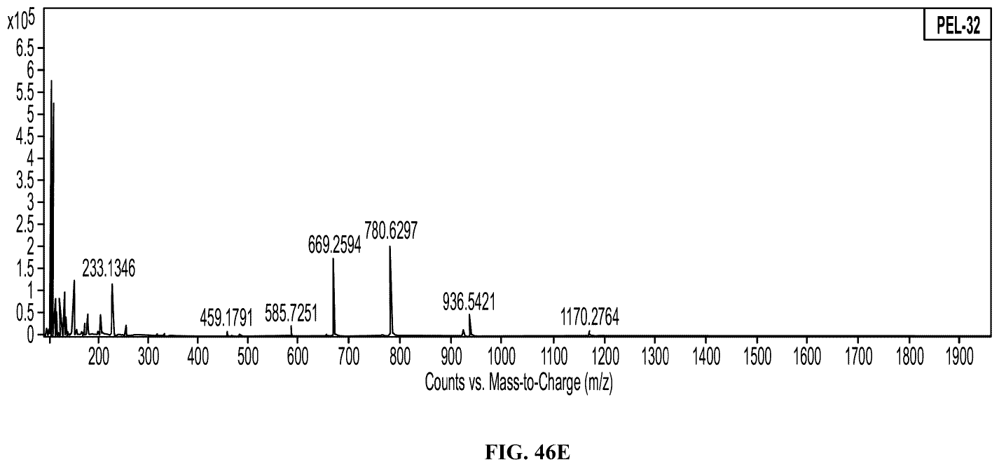

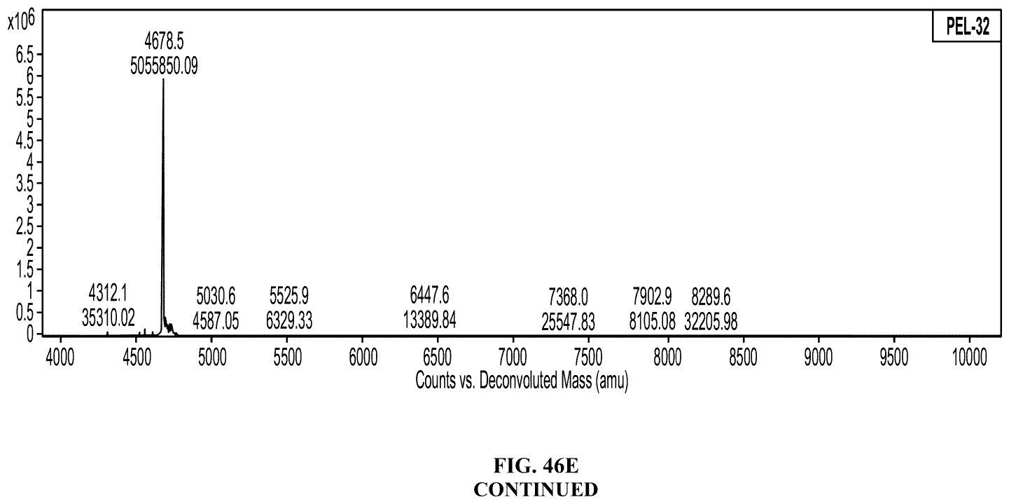

Synthesized electrophile-attached Z33 reagents are listed in Table 4 The ADC mass analysis results for the synthesized electrophile-attached Z33 reagents are shown in FIGS. 46A to 46E. “Hcy” refers to homocysteine.

TABLE 4
Characteristics of electrophile-attached Z33 reagents.

| | | SEQ ID | | Calculated | Observed |
|---|---|---|---|---|---|
| Code | Sequence^{a} | NO: | Electrophile | Mass | Mass^{c} |
| PEL- 01 | FNMQQQRRFYEALHD PNLNCEQRNAKIKSI RDD | 14 | 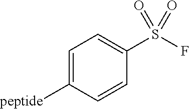 | 4236.7 | 4237.2 |
| PEL- 02 | FNMQQQRRFYEALHD PNLNCEQRNAKIKSI RDD | 14 | 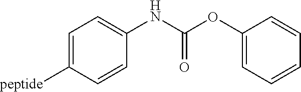 | 4289.8 | 4290.5 |
| PEL- 03 | FNMQQQRRFYEALHD PNLNCEQRNAKIKSI RDD | 14 | 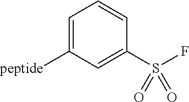 | 4236.7 | 4236.7 |
| PEL- 04 | FNMQQQRRFYEALHD PNLNCEQRNAKIKSI RDD | 14 | 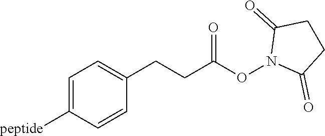 | 4323.8 | 4323.7 |
| PEL- 05 | FNMQQQRRFYEALHD PNLNHcyEQRNAKIKSI RDD | 19 | 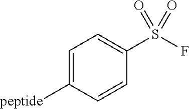 | 4250.7 | 4251.1 |
| PEL- 06 | FNMQQQRRFYEALHD PNLNHcyEQRNAKIKSI RDD | 19 | 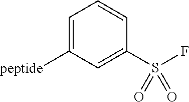 | 4250.7 | 4251.1 |
| PEL- 07 | FNMQQQRRFYEALHD PNLNHcyEQRNAKIKSI RDD | 19 | 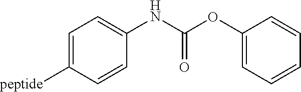 | 4303.6 | 4304.2 |
| PEL- 08 | FNMQQQRRFYEALHD PNLNHcyEQRNAKIKSI RDD | 19 | 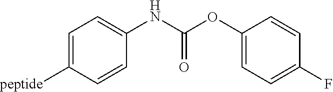 | 4321.6 | 4322.2 |
| PEL- 09 | 5-azido-pentanoyl- FNMQQQRRFYEALHD PNLNHcyEQRNAKIKSI RDD | 19 | 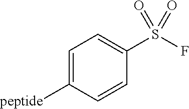 | 4375.6 | 4376.3 |
| PEL- 10 | CFNMQQQRRFYEALH DPNLNEEQRNAKIKS IRDD | 11 | 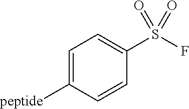 | 4365.8 | 4366.2 |
| PEL- 11 | FNCQQQRRFYEALHD PNLNEEQRNAKIKSI RDD | 12 | 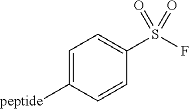 | 4234.6 | 4235.4 |
| PEL- 12 | FNMQQQRRFYEALHD PCLNEEQRNAKIKSI RDD | 13 | 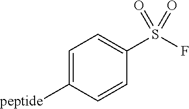 | 4251.7 | 4252.2 |
| PEL- 13 | FNMQQQRRFYEALHD PNLNECQRNAKIKSI RDD | 15 | 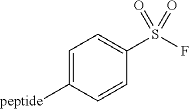 | 4236.7 | 4237.2 |
| PEL- 14 | FNMQQQRRFYEALHD PNLNEEQRCAKIKSI RDD | 16 | 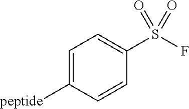 | 4251.7 | 4252.1 |
| PEL- 15 | FNMQQQRRFYEALHD PNLNEEQRNAKIKSI CDD | 17 | 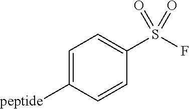 | 4209.6 | 4210.4 |
| PEL- 16 | FNMQQQRRFYEALHD PNLNCEQRNAKIKSI RDD | 14 | 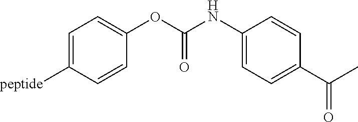 | 4331.9 | 4332.2 |
| PEL- 17 | FNMQQQRRFYEALHD PNLNHcyEQRNAKIKSI RDD | 19 | 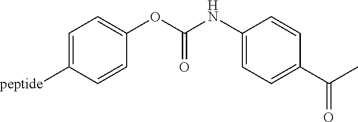 | 4345.7 | 4346.2 |
| PEL- 18 | FNMQQQRRFYEALHD PNLNHcyEQRNAKIKSI RDD | 19 | 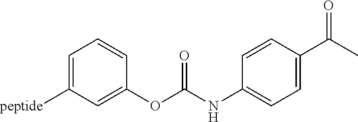 | 4345.7 | 4345.8 |
| PEL- 19 | FNMQQQRRFYEALHD PNLNCEQRNAKIKSI RDD | 14 | 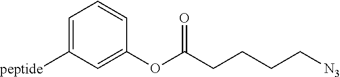 | 4295.8 | 4296.1 |
| PEL- 20 | FNMQQQRRFYEALHD PNLNHcyEQRNAKIKSI RDD | 19 | 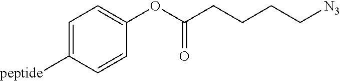 | 4309.6 | 4309.7 |
| PEL- 21 | FNMQQQRRFYEALHD PNLNHcyEQRNAKIKSI RDD | 19 | 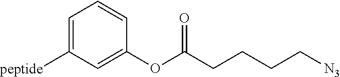 | 4309.6 | 4310.1 |
| PEL- 22 | FNMQQQRRFYEALHD PNLNHcyEQRNAKIKSI RDD | 19 | 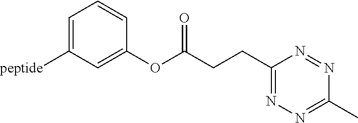 | 4334.6 | 4334.7 |
| PEL- 23 | FNCQQQRRFYEALHD PNLNEEQRNAKIKSI RDD | 12 | 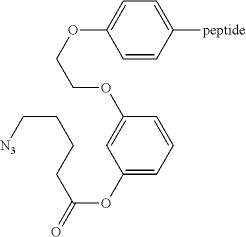 | 4429.9 | 4430.2 |
| PEL- 24 | FNHcyQQQRRFYEALHD PNLNEEQRNAKIKSI RDD | 22 | 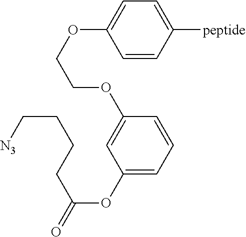 | 4443.9 | 4443.5 |
| PEL- 25 | FNCQQQRRFYEALHD PNLNEEQRNAKIKSI RDD | 12 | 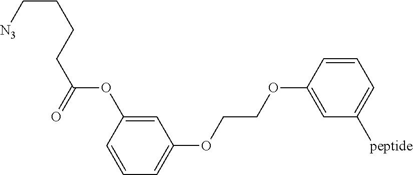 | 4429.9 | 4430.4 |
| PEL- 26 | FNHcyQQQRRFYEALHD PNLNEEQRNAKIKSI RDD | 2 | 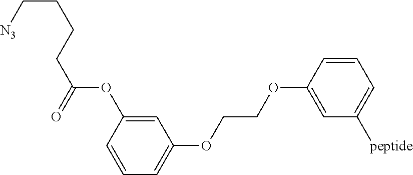 | 4443.9 | 4444.3 |
| PEL- 27 | FNMQQQRRFYEALHD PNLNEEQRNAKIKSI CDD | 17 | 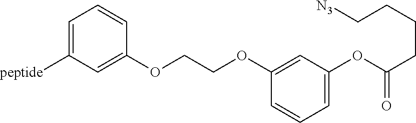 | 4404.9 | 4405.5 |
| PEL- 28 | FNMQQQRRFYEALHD PNLNEEQRNAKIKSI RCD | 18 | 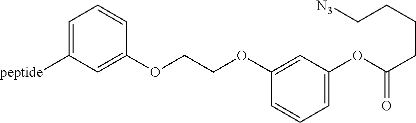 | 4446.0 | 4446.3 |
| PEL- 29 | FNMQQQRRFYEALHD PNLNEEQRNAKIKSI RHcyD | 21 | 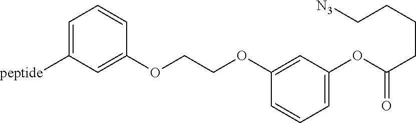 | 4460.0 | 4459.7 |
| PEL- 30 | H₂N-PEG8- FNMQQQRRFYEALHD PNLNHcyEQRNAKIKSI RDD | 19 | 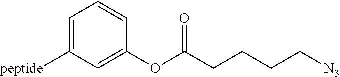 | 4733.1 | 4733.4 |
| PEL- 31 | FNMQQQRRFYEALHD pNLNCEQRNAKIKSI RDD | 14 | 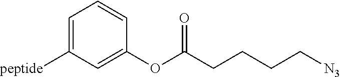 | 4295.8 | 4296.1 |
| PEL- 32 | FNMQQQRRFYEALHD PNLNHcyEQRNAKIKSI RDD | 19 | 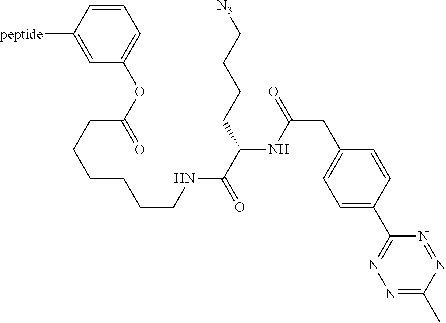 | 4678.1 | 4678.5 |

^{a}Electrophile-attached amino acids are highlighted in bold.,
^{b}average molecular mass,
^{c}after deconvolution

### Synthesis of PEL-01

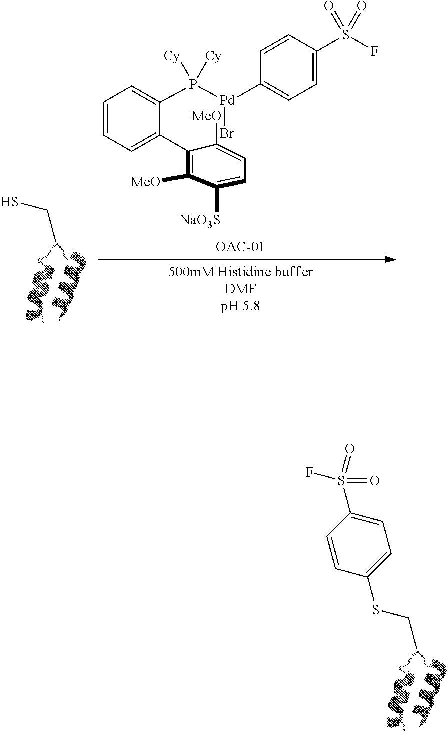

PEP-04 (5.0 mg) was weighed into a 50 mL centrifuge tube, and Histidine buffer (500 mM, pH 5.8, 6 mL) was added. The mixture was then stirred by hand to obtain a homogeneous solution. Additionally, OAC-01 (2.3 mg) was weighed into a 0.6 mL microcentrifuge tube, and DMF (0.18 mL) was added to prepare a separate brown solution using sonication. The OAC-01 solution was added to the PEP-04 solution, and the mixture was vortexed briefly and allowed to stand at room temperature for 2 hours. Subsequently, the reaction solution was directly loaded onto a Biotage® Sfär C18 D column (12 g) and purified using the following conditions: 5 CV 5% B, 1 CV 5-10% B, 20 CV 10-35% B. 1 μL of a 20-fold diluted solution of the obtained fractions was analyzed by LC-MS, and the fractions containing only the target product were collected and lyophilized to obtain PEL-01 (1.3 mg) as a white solid.

### Synthesis of PEL-02

PEP-04 (5.0 mg) and OAC-02 (2.2 mg) were used to synthesize PEL-02 following the same procedure as for PEL-01. PEL-02 (0.82 mg) was obtained as a white solid.

### Synthesis of PEL-03

PEP-04 (5.0 mg) and OAC-03 (2.1 mg) were used to synthesize PEL-03 following the same procedure as for PEL-01. PEL-03 (2.2 mg) was obtained as a white solid.

### Synthesis of PEL-04

PEP-04 (5.0 mg), OAC-04 (2.3 mg), and phosphate buffer (20 mM, pH 7.2) were used to synthesize PEL-04 following the same procedure as for PEL-01. PEL-04 (0.41 mg) was obtained as a white solid.

### Synthesis of PEL-05

PEP-09 (5.0 mg) and OAC-01 (2.1 mg) were used to synthesize PEL-05 following the same procedure as for PEL-01. PEL-05 (2.8 mg) was obtained as a white solid.

### Synthesis of PEL-06

PEP-09 (5.0 mg) and OAC-03 (2.1 mg) were used to synthesize PEL-06 following the same procedure as for PEL-01. PEL-06 (3.3 mg) was obtained as a white solid.

### Synthesis of PEL-07

PEP-09 (5.0 mg) and OAC-02 (2.2 mg) were used to synthesize PEL-07 following the same procedure as for PEL-01. PEL-07 (2.1 mg) was obtained as a white solid.

### Synthesis of PEL-08

PEP-09 (5.0 mg) and OAC-05 (2.3 mg) were used to synthesize PEL-08 following the same procedure as for PEL-01. PEL-08 (2.1 mg) was obtained as a white solid.

### Synthesis of PEL-09

PEP-11 (5.0 mg) and OAC-01 (2.3 mg) were used to synthesize PEL-09 following the same procedure as for PEL-01. PEL-09 (2.1 mg) was obtained as a white solid.

### Synthesis of PEL-10

PEP-01 (5.0 mg) and OAC-01 (2.3 mg) were used to synthesize PEL-10 following the same procedure as for PEL-01. PEL-10 (1.3 mg) was obtained as a white solid.

### Synthesis of PEL-11

PEP-02 (5.0 mg) and OAC-01 (2.3 mg) were used to synthesize PEL-11 following the same procedure as for PEL-01. PEL-11 (0.45 mg) was obtained as a white solid.

### Synthesis of PEL-12

PEP-03 (5.0 mg) and OAC-01 (2.3 mg) were used to synthesize PEL-12 following the same procedure as for PEL-01. PEL-12 (4.9 mg) was obtained as a white solid.

### Synthesis of PEL-13

PEP-05 (5.0 mg) and OAC-01 (2.3 mg) were used to synthesize PEL-13 following the same procedure as for PEL-01. PEL-13 (2.8 mg) was obtained as a white solid.

### Synthesis of PEL-14

PEP-06 (5.0 mg) and OAC-01 (2.3 mg) were used to synthesize PEL-14 following the same procedure as for PEL-01. PEL-14 (1.3 mg) was obtained as a white solid.

### Synthesis of PEL-15

PEP-07 (5.0 mg) and OAC-01 (2.3 mg) were used to synthesize PEL-15 following the same procedure as for PEL-01. PEL-15 (2.7 mg) was obtained as a white solid.

### Synthesis of PEL-16

PEP-04 (5.0 mg) and OAC-06 (2.3 mg) were used to synthesize PEL-16 following the same procedure as for PEL-01. PEL-16 (1.6 mg) was obtained as a white solid.

### Synthesis of PEL-17

PEP-09 (5.0 mg) and OAC-06 (2.3 mg) were used to synthesize PEL-17 following the same procedure as for PEL-01. PEL-17 (3.0 mg) was obtained as a white solid.

### Synthesis of PEL-18

PEP-09 (5.0 mg) and OAC-07 (2.5 mg) were used to synthesize PEL-18 following the same procedure as for PEL-01. PEL-18 (2.5 mg) was obtained as a white solid.

### Synthesis of PEL-19

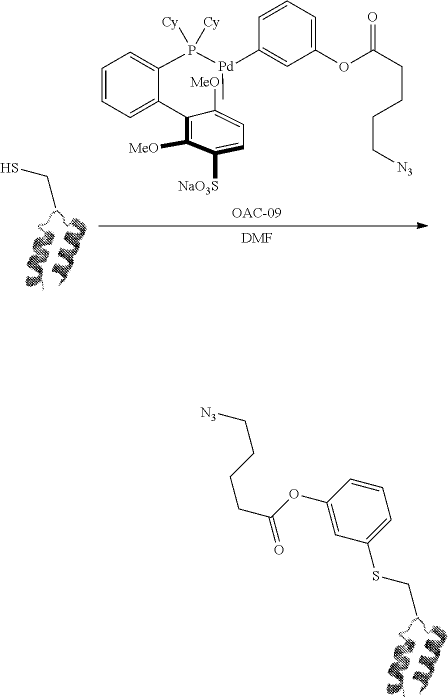

PEP-04 (5.0 mg) was weighed into a 1.5 mL microcentrifuge tube, and DMF (200 μL) was added. Additionally, OAC-09 (2.3 mg) was weighed into a 0.6 mL microcentrifuge tube, and DMF (0.18 mL) was added to prepare a separate brown solution using sonication. The OAC-solution was added to the PEP-04 solution, and the mixture was vortexed briefly and allowed to stand at room temperature for 30 minutes. Then the reaction mixture was transferred to a 50 mL centrifuge tube and diluted to 5 mL with water. Subsequently, the reaction solution was directly loaded onto a Biotage® Sfär C18 D column (12 g) and purified using the following conditions: 5 CV 5% B, 1 CV 5-10% B, 20 CV 10-35% B. 1 μL of a 20-fold diluted solution of the obtained fractions was analyzed by LC-MS, and the fractions containing only the target product were collected and lyophilized to obtain PEL-019 (2.8 mg) as a white solid.

### Synthesis of PEL-20

PEP-09 (5.0 mg) and OAC-08 (2.2 mg) were used to synthesize PEL-20 following the same procedure as for PEL-01. PEL-20 (3.4 mg) was obtained as a white solid.

### Synthesis of PEL-21

PEP-09 (15 mg) and OAC-09 (7.1 mg) were used to synthesize PEL-21 following the same procedure as for PEL-19. PEL-21 (10 mg) was obtained as a white solid.

### Synthesis of PEL-22

PEP-09 (5.0 mg) and OAC-10 (2.4 mg) were used to synthesize PEL-22 following the same procedure as for PEL-19. PEL-22 (1.2 mg) was obtained as a white solid.

### Synthesis of PEL-23

PEP-02 (5.0 mg) and OAC-11 (2.6 mg) were used to synthesize PEL-23 following the same procedure as for PEL-01. PEL-23 (3.2 mg) was obtained as a white solid.

### Synthesis of PEL-24

PEP-09 (5.0 mg) and OAC-11 (2.6 mg) were used to synthesize PEL-24 following the same procedure as for PEL-01. PEL-24 (3.1 mg) was obtained as a white solid.

### Synthesis of PEL-25

PEP-02 (5.0 mg) and OAC-12 (2.6 mg) were used to synthesize PEL-25 following the same procedure as for PEL-01. PEL-25 (3.2 mg) was obtained as a white solid.

### Synthesis of PEL-26

PEP-09 (5.0 mg) and OAC-12 (2.6 mg) were used to synthesize PEL-26 following the same procedure as for PEL-01. PEL-26 (4.5 mg) was obtained as a white solid.

### Synthesis of PEL-27

PEP-07 (5.0 mg) and OAC-12 (2.6 mg) were used to synthesize PEL-27 following the same procedure as for PEL-01. PEL-27 (3.5 mg) was obtained as a white solid.

### Synthesis of PEL-28

PEP-08 (5.0 mg) and OAC-12 (2.6 mg) were used to synthesize PEL-28 following the same procedure as for PEL-01. PEL-28 (0.5 mg) was obtained as a white solid.

### Synthesis of PEL-29

PEP-13 (5.0 mg) and OAC-12 (2.6 mg) were used to synthesize PEL-29 following the same procedure as for PEL-19. PEL-29 (0.48 mg) was obtained as a white solid.

### Synthesis of PEL-30

PEP-11 (15.7 mg) and OAC-09 (6.7 mg) were used to synthesize PEL-30 following the same procedure as for PEL-19. PEL-30 (9.5 mg) was obtained as a white solid.

### Synthesis of PEL-31

PEP-12 (15.0 mg) and OAC-09 (7.1 mg) were used to synthesize PEL-31 following the same procedure as for PEL-19. PEL-31 (6.7 mg) was obtained as a white solid.

### Synthesis of PEL-32

PEP-09 (5.0 mg) and OAC-13 (3.3 mg) were used to synthesize PEL-32 following the same procedure as for PEL-19. PEL-32 (0.32 mg) was obtained as a white solid.

### Example 6. Conjugation Studies on IgG

### Conjugation of Z33 Analog to Trastuzumab

### General Reaction Procedure

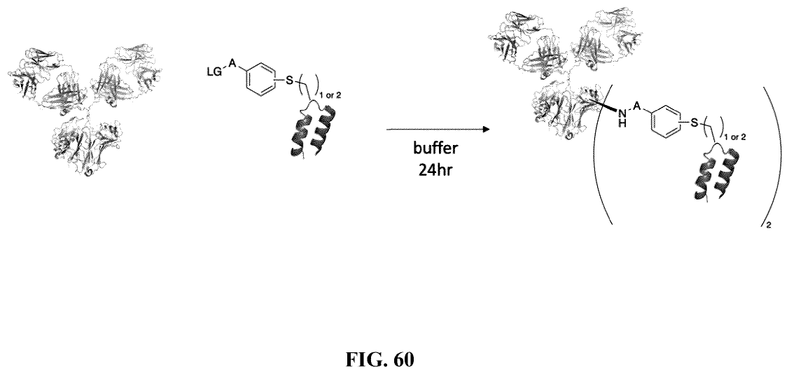

Reactions were performed on a 20 μL scale using 0.2 mL PCR tubes (FIG. 60). First, the electrophile-attached Z33 peptide reagent was dissolved in water to prepare a 1 mg/mL solution. Trastuzumab (Syd Labs Inc., 52 mg/mL), electrophile-attached Z33 peptide reagent (1 mg/mL), and buffer were added to achieve final concentrations of 10 uM, 200 uM, and values listed in the result table, respectively. The contents were mixed using a 10 μL pipette and incubated at room temperature in the dark for 24 hours. The resulting reaction solution was quenched with a 100 mM glycine solution and analyzed by SDS-PAGE.

### Large Scale Reaction Procedure for Entry 15

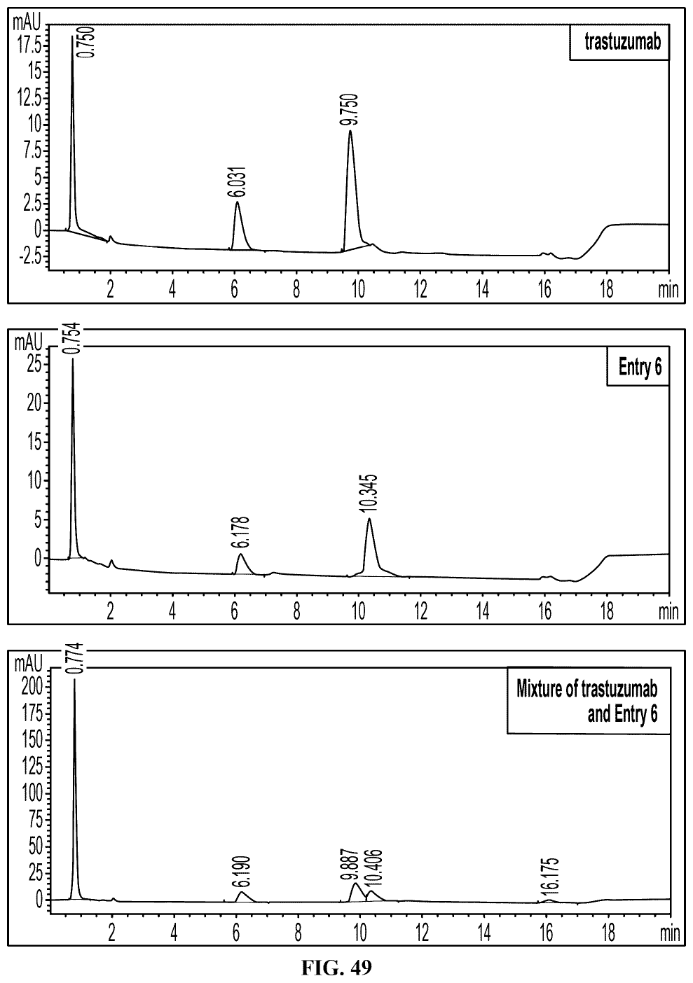

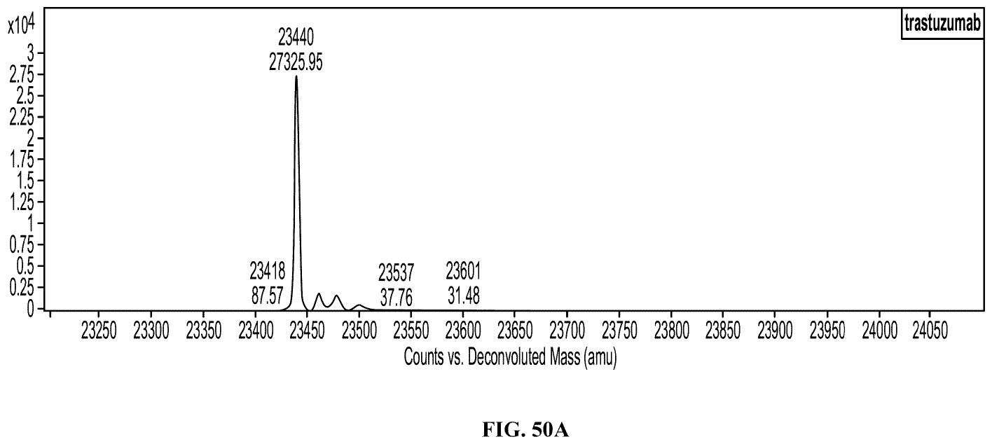

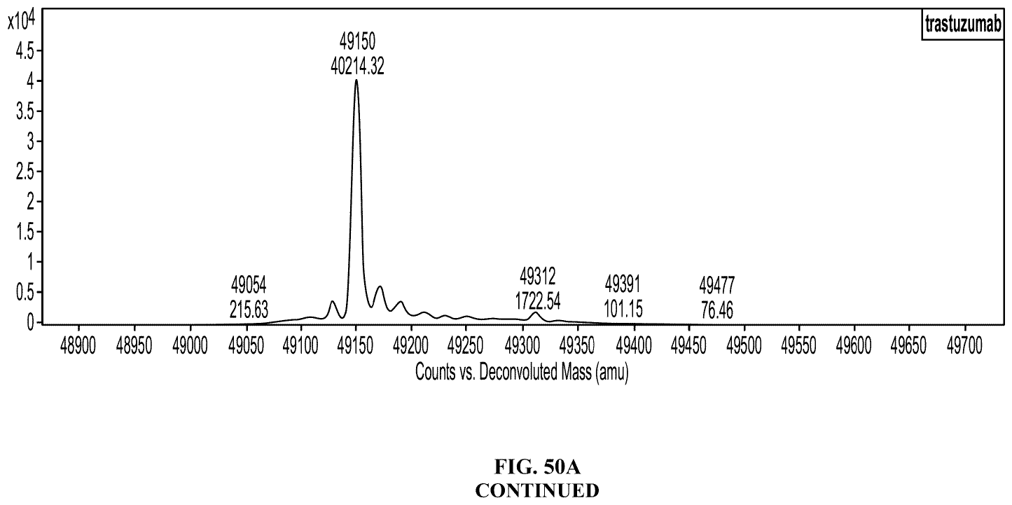

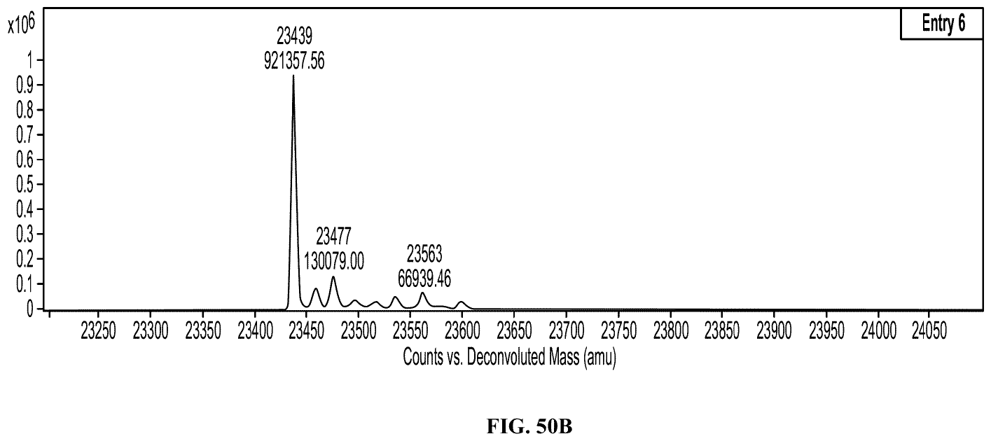

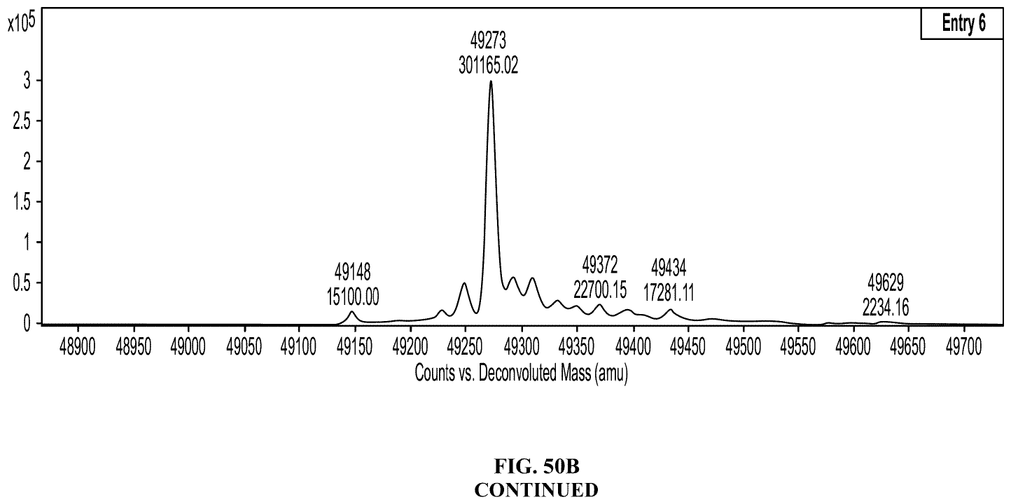

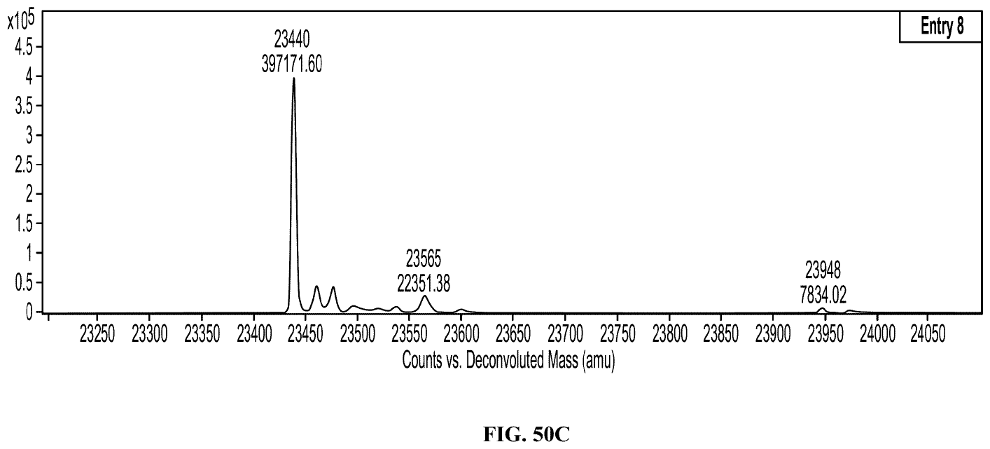

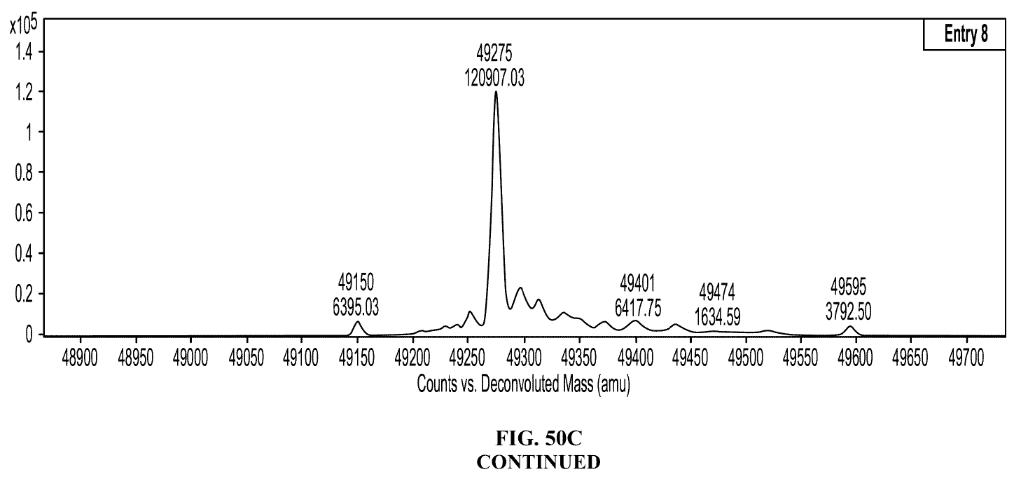

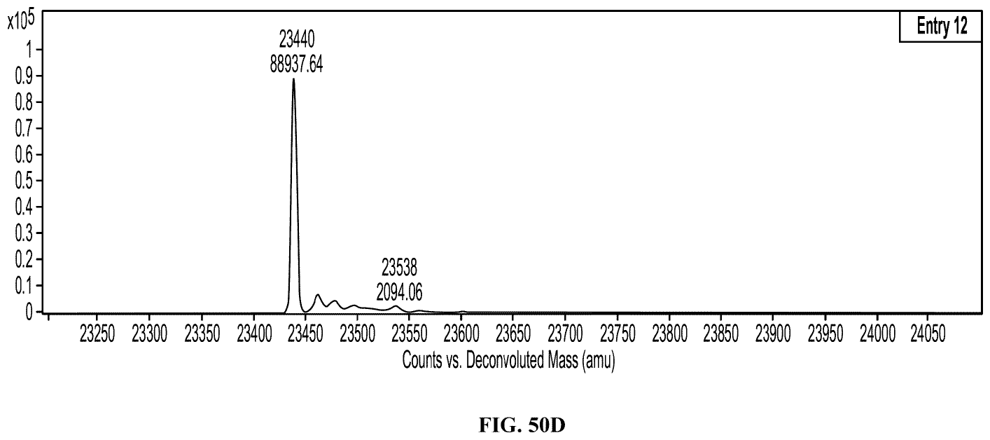

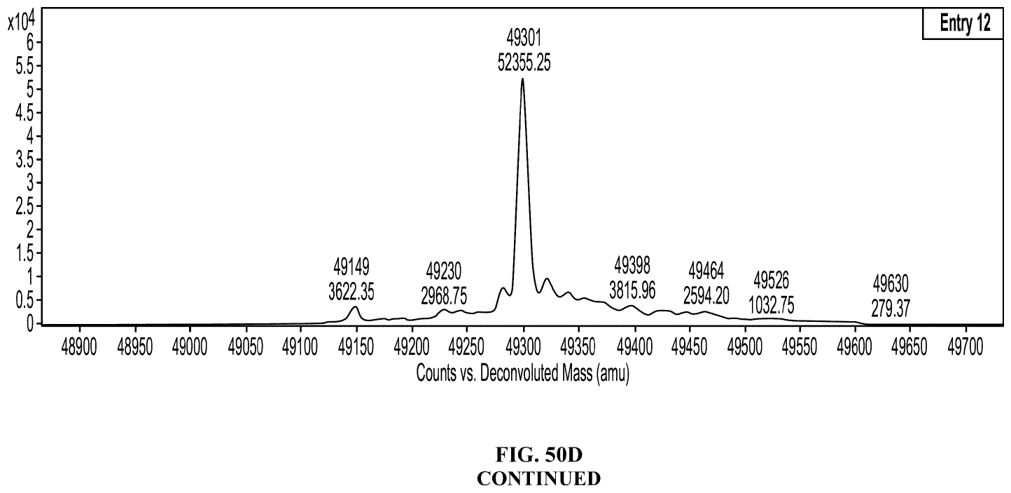

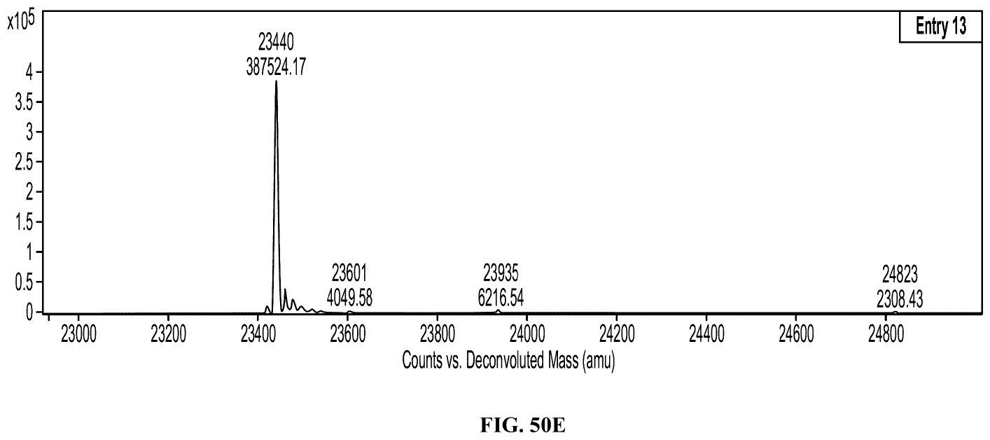

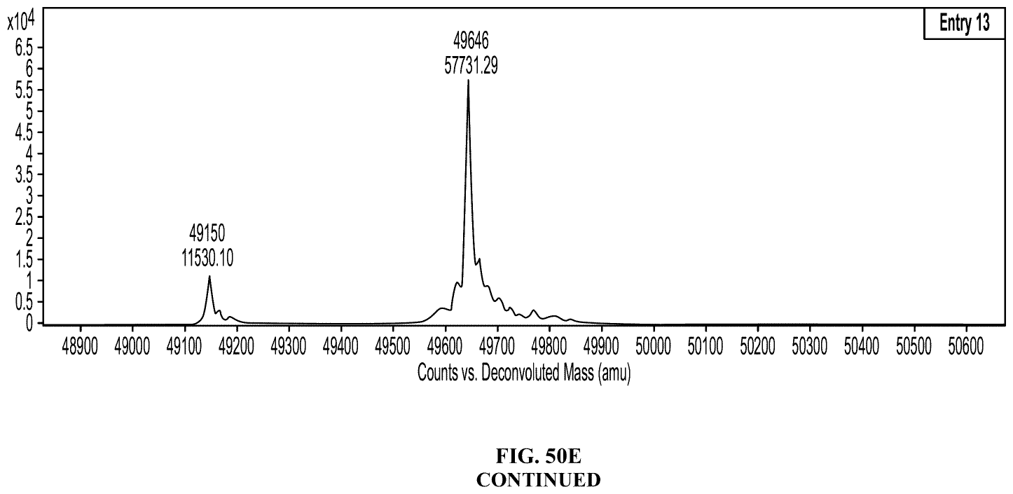

The reaction was performed on a 210 μL scale using a 1.5 mL microcentrifuge tube. First, PEL-09 was dissolved in water to prepare a 2 mg/mL solution. Trastuzumab (Bio X Cell, 9.2 mg/mL, 32.2 μL), PEL-09 (2 mg/mL, 43.8 μL), HEPES buffer (161 mM, 124 μL, pH 8.7), and NaCl solution (4M, 10 μL) were added and mixed using a 20 μL pipette, followed by incubation at room temperature in the dark for 24 hours. The reaction mixture was diluted to 400 μL with citrate buffer (100 mM, pH 2.7) and filtered through an Amicon® filter (0.5 mL, 50KMWCO) at 14000 ref for 7 minutes. After filtration, 400 μL of citrate buffer (100 mM, pH 2.68) was added and centrifuged again, followed by centrifugation with 400 μL of Tris (100 mM, pH 8.1) buffer (total of 3 centrifugations). Finally, the remaining solution (roughly 20 μL) on the Amicon® filter was transferred to a microcentrifuge tube, and the Amicon® filter was washed twice with 40 μL of Tris buffer (100 mM, pH 8.1). The obtained solution was analyzed by LC-MS (FIG. 49, Table 6) and SDS-PAGE (FIG. 50, Table 5).

### General Procedure of SDS-PAGE Analysis

The reaction solution corresponding to 3.5 μg of IgG was taken into a PCR tube and diluted with Tris buffer (100 mM, pH 8.1) to a final volume of 3 μL. Then, Laemmli sample buffer (BIO-RAD, 2×+5v/v % 2-mercaptoethanol, 3 μL) was added, and the mixture was heated at 70° C. for 5 minutes. After returning the reaction solution to room temperature, 2 μL was loaded onto an SDS-Gel. Subsequently, electrophoresis, washing, staining, and analysis were performed using the methods described in General Information Section.

### General Procedure of LC-MS Analysis

A reaction solution corresponding to 9.0 μg of IgG was taken into a PCR tube and diluted with Tris buffer (100 mM, pH 8.1) to a final volume of 6 μL. Separately, a mixed solution of PNGase F (New England BioLabs, 63 units) and Glycoprotein Denaturing Buffer (New England Biolabs, 1×, 4 μL) was prepared and added to the reaction solution, followed by incubation at 37° C. for 4 to 12 hours. Afterward, DTT (200 mM in water, 2 μL) was added, and the mixture was further incubated at 37° C. for 1 hour. Finally, the solution was diluted to a volume of 40 μL with a mixture of water and acetonitrile (95:5), and 8 μL of the diluted solution was injected into LC-MS.

TABLE 5
IgG conjugation study results analyzed by SDS-PAGE.

| | | | | | | Result analyzed by SDS-PAGE | | |
|---|---|---|---|---|---|---|---|---|
| | | | | | | | | Heavy |
| | | | | | | | | chain total |
| | | | | SEQ | | Light chain | Heavy | conversion |
| | PEL | | Z33 peptide | ID | reaction | total | chain total | ratio |
| Entry | # | Linkage structure | sequence | NO: | buffer | conversion | conversion | (+1/+2/+3) |
| 1 | 01 | 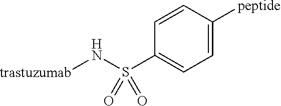 | FNMQQQRRFY EALHDPNLNC EQRNAKIKSI RDD | 14 | 100 mM HEPES pH8.5 | <1% | 77% | 94/6/0 |
| 2 | 01 | 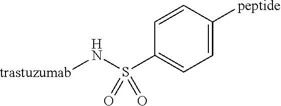 | FNMQQQRRFY EALHDPNLNC EQRNAKIKSI RDD | 14 | 100 mM phosphate pH7.2 | <1% | 49% | 100/0/0 |
| 3^{a} | 02 | 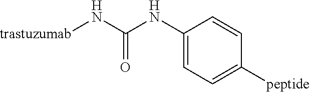 | FNMQQQRRFY EALHDPNLNC EQRNAKIKSI RDD | 14 | 20 mM HEPES pH8.5 | 20% | 50% | 77/23/0 |
| 4^{b} | 03 | 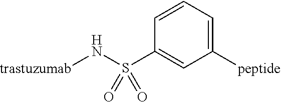 | FNMQQQRRFY EALHDPNLNC EQRNAKIKSI RDD | 14 | 20 mM HEPES pH8.5 | <1% | 81% | 83/17/0 |
| 5 | 04 | 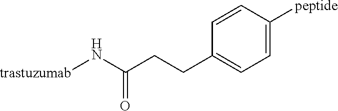 | FNMQQQRRFY EALHDPNLNC EQRNAKIKSI RDD | 14 | 100 mM HEPES pH8.5 | <1% | 49% | 100/0/0 |
| 6 | 04 | 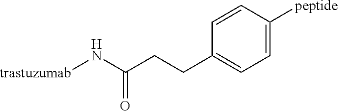 | FNMQQQRRFY EALHDPNLNC EQRNAKIKSI RDD | 14 | 20 mM phosphate pH7.2 | <1% | 37% | 100/0/0 |
| 7 | 05 | 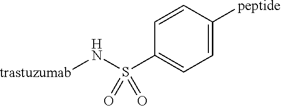 | FNMQQQRRFY EALHDPNLNHcy EQRNAKIKSI RDD | 19 | 100 mM HEPES pH8.5 | <1% | 95%< | 94/6/0 |
| 8 | 05 | 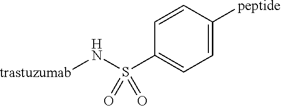 | FNMQQQRRFY EALHDPNLNHcy EQRNAKIKSI RDD | 19 | 100 mM phosphate pH7.2 | <1% | 97% | 99/1/0 |
| 9 | 06 | 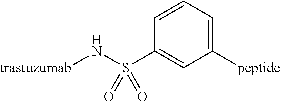 | FNMQQQRRFY EALHDPNLNHcy EQRNAKIKSI RDD | 19 | 100 mM HEPES pH8.5 | <1% | 95%< | 91/9/0 |
| 10 | 06 | 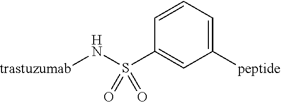 | FNMQQQRRFY EALHDPNLNHcy EQRNAKIKSI RDD | 19 | 100 mM phosphate pH7.2 | <1% | 88% | 99/1/0 |
| 11 | 07 | 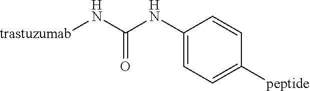 | FNMQQQRRFY EALHDPNLNHcy EQRNAKIKSI RDD | 19 | 100 mM HEPES pH8.5 | <1% | 89% | 97/3/0 |
| 12 | 07 | 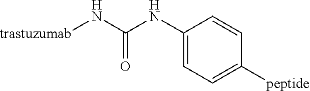 | FNMQQQRRFY EALHDPNLNHcy EQRNAKIKSI RDD | 19 | 100 mM phosphate pH7.2 | <1% | 17% | 100/0/0 |
| 13 | 08 | 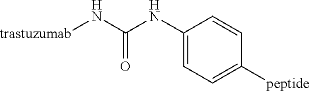 | FNMQQQRRFY EALHDPNLNHcy EQRNAKIKSI RDD | 19 | 100 mM HEPES pH8.5 | <1% | 89% | 97/3/0 |
| 14 | 08 | 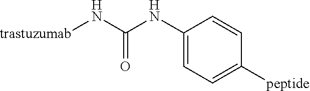 | FNMQQQRRFY EALHDPNLNHcy EQRNAKIKSI RDD | 19 | 100 mM phosphate pH7.2 | <1% | 17% | 100/0/0 |
| 15 | 09 | 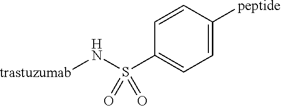 | 5-azido-pentanoyl- FNMQQQRRFY EALHDPNL NHcy EQRNAKIKSI RDD | 19 | 100 mM HEPES pH8.7 | <1% | 95%< | 98/2/0 |
| 16 | 10 | 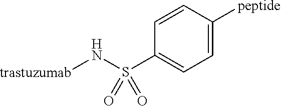 | CFNMQQQRRF YEALHDPNLN EEQRNAKIKS IRDD | 11 | 100 mM HEPES pH8.5 | <1% | 44% | 90/10/0 |
| 17 | 10 | 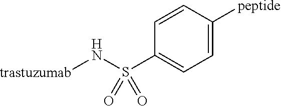 | CFNMQQQRRF YEALHDPNLN EEQRNAKIKS IRDD | 11 | 100 mM phosphate pH7.2 | <1% | 22% | 100/0/0 |
| 18 | 11 | 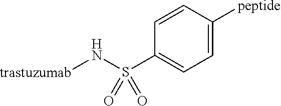 | FNCQQQRRFY EALHDPNLNE EQRNAKIKSI RDD | 12 | 100 mM HEPES pH8.5 | <1% | 32% | 42/44/14 |
| 19 | 11 | 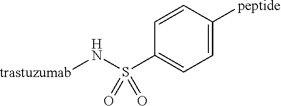 | FNCQQQRRFY EALHDPNLNE EQRNAKIKSI RDD | 12 | 100 mM phosphate pH7.2 | <1% | 12% | 57/43/0 |
| 20 | 12 | 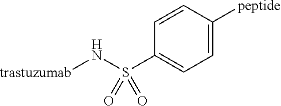 | FNMQQQRRFY EALHDPCLNE EQRNAKIKSI RDD | 13 | 100 mM HEPES pH8.5 | <1% | 30% | 100/0/0 |
| 21 | 12 | 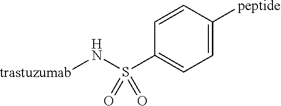 | FNMQQQRRFY EALHDPCLNE EQRNAKIKSI RDD | 13 | 100 mM phosphate pH7.2 | <1% | 10% | 100/0/0 |
| 22 | 13 | 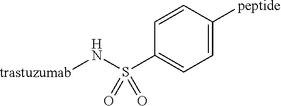 | FNMQQQRRFY EALHDPNLNE CORNAKIKSI RDD | 15 | 100 mM HEPES pH8.5 | <1% | 12% | 100/0/0 |
| 23 | 13 | 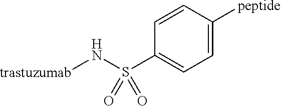 | FNMQQQRRFY EALHDPNLNE CORNAKIKSI RDD | 15 | 100 mM phosphate pH7.2 | <1% | 4% | 100/0/0 |
| 24 | 14 | 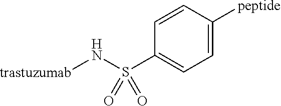 | FNMQQQRRFY EALHDPNLNE EQRCAKIKSI RDD | 16 | 100 mM HEPES pH8.5 | <1% | 8% | 100/0/0 |
| 25 | 14 | 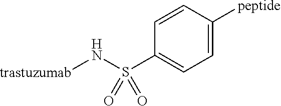 | FNMQQQRRFY EALHDPNLNE EQRCAKIKSI RDD | 16 | 100 mM phosphate pH7.2 | <1% | 4% | 100/0/0 |
| 26 | 15 | 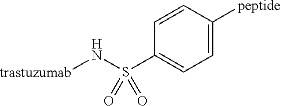 | FNMQQQRRFY EALHDPNLNE EQRNAKIKSI CDD | 17 | 100 mM HEPES pH8.5 | <1% | 9% | 100/0/0 |
| 27 | 15 | 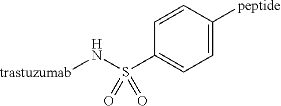 | EALHDPNLNE FNMQQQRRFY EQRNAKIKSI CDD | 17 | phosphate pH7.2 100 mM | <1% | 0% | |

^{a}Reaction was conducted at 37° C. using the 8 equivalents of the reagent.
^{b}200 mM NaCl was added to the reaction.

TABLE 6
IgG conjugation study results analyzed by q-ToF LC-MS.

| | | | | | Result analyzed by Q-ToF LC-MS | | |
|---|---|---|---|---|---|---|---|
| | | | | | | | Heavy |
| | | | | | Light | | chain total |
| | | | | | chain | Heavy | conversion |
| | PEL | | Z33 peptide | reaction | total | chain total | ratio |
| Entry | # | Linkage structure | sequence | buffer | conversion | conversion | (+1/+2/+3) |
| 15 | 09 | 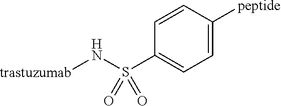 | 5-azido-pentanoyl- FNMQQQRRFY EALHDPNLNHcy EQRNAKIKSI RDD (SEQ ID NO: 19) | 100 mM HEPES pH8.7 | <1% | 95% | 100/0/0 |

### Small Bioconjugation Handles Installation to Trastuzumab—Using the Electrophile-Attached Z33E20C/Hcy Reagents

### General Reaction Procedure

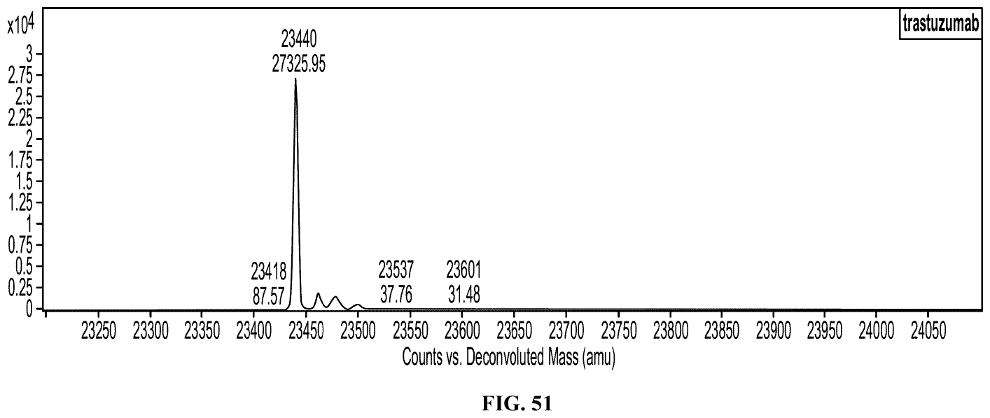

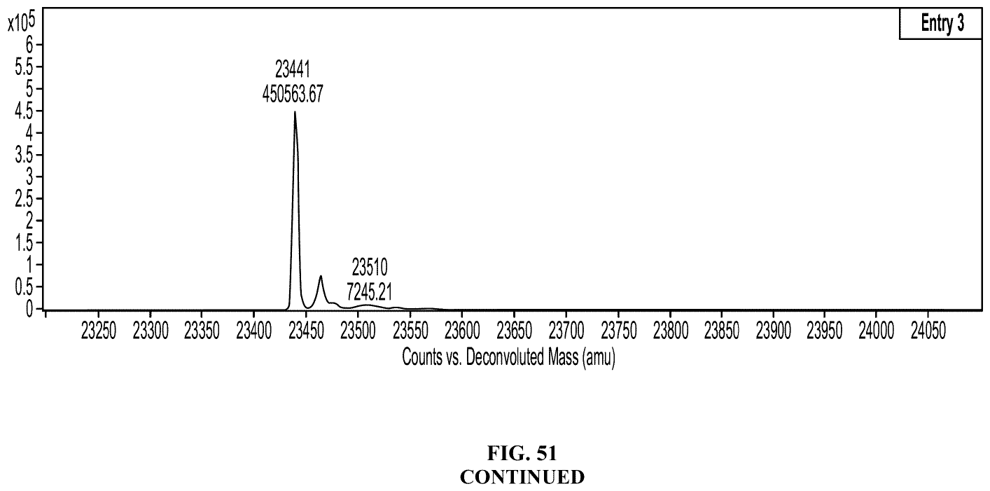

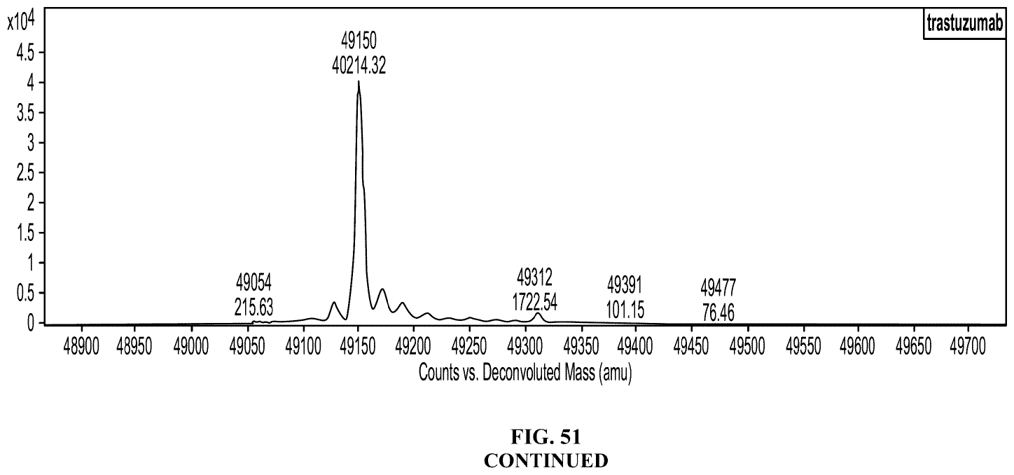

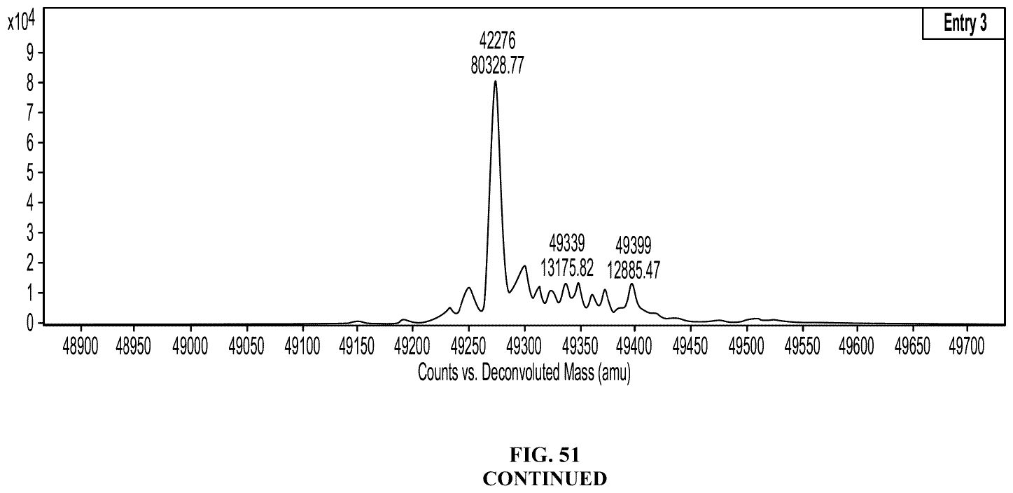

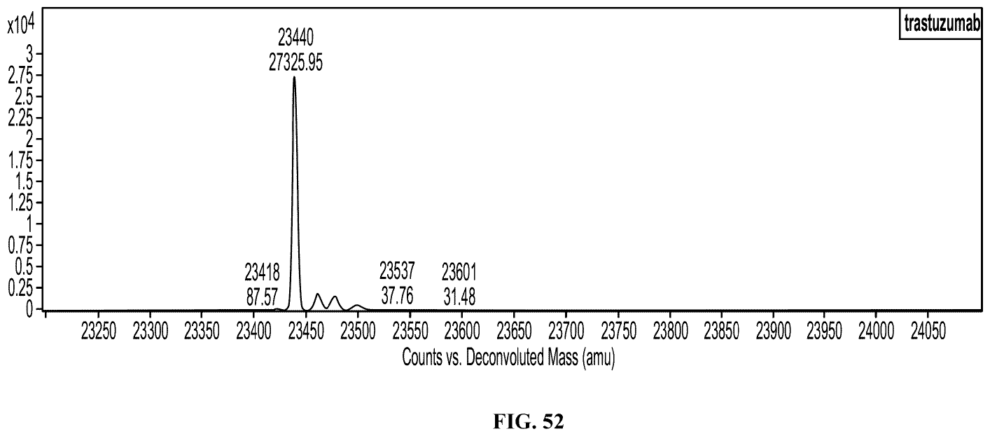

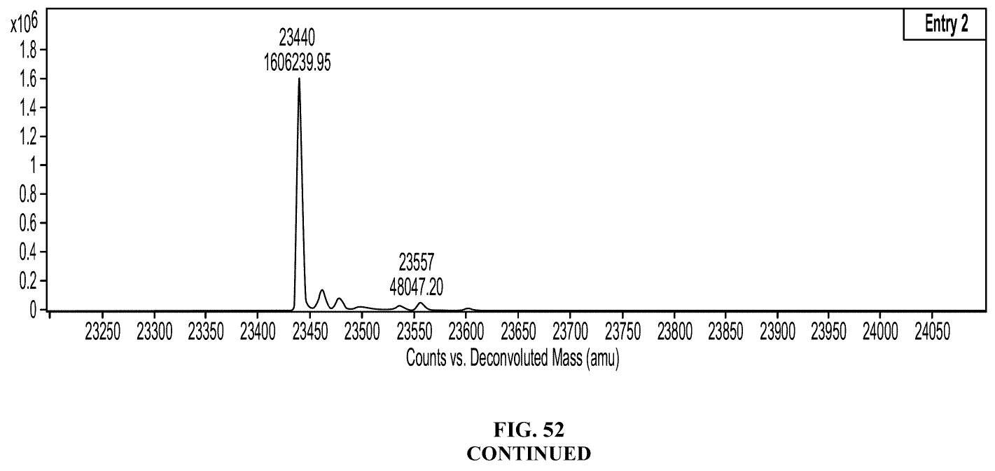

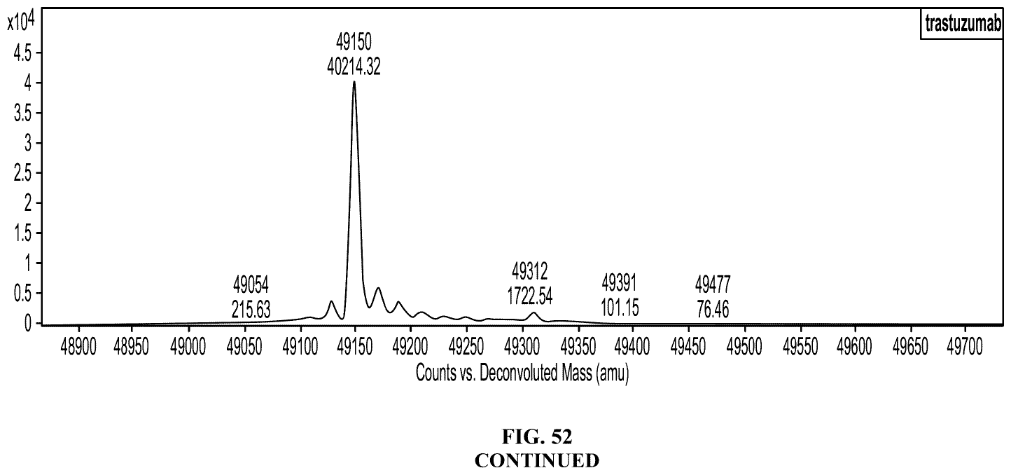

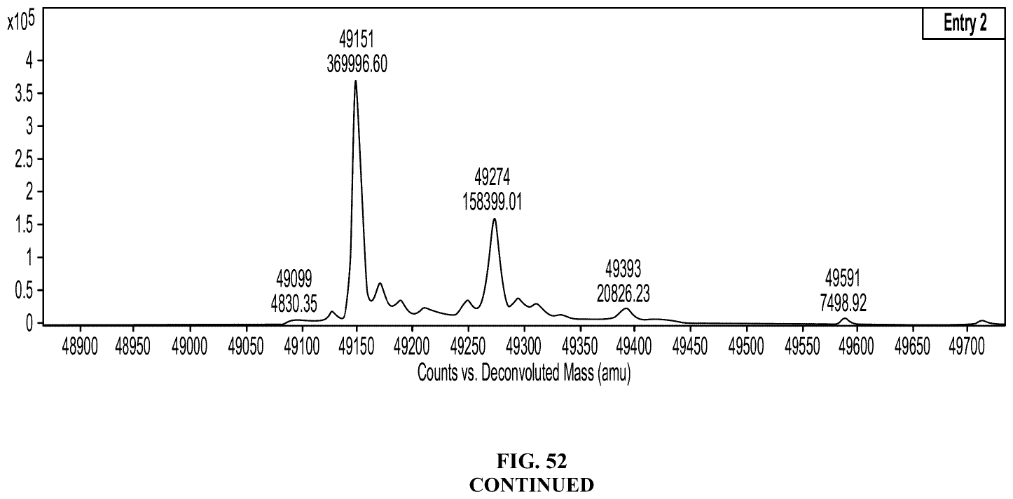

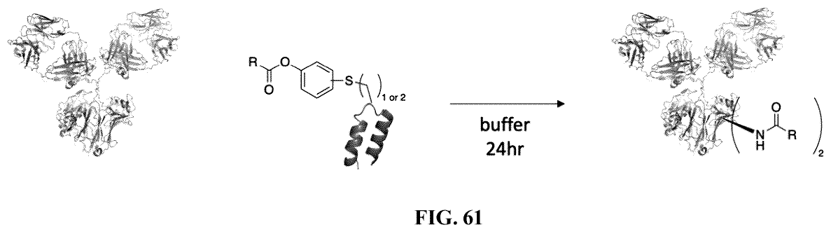

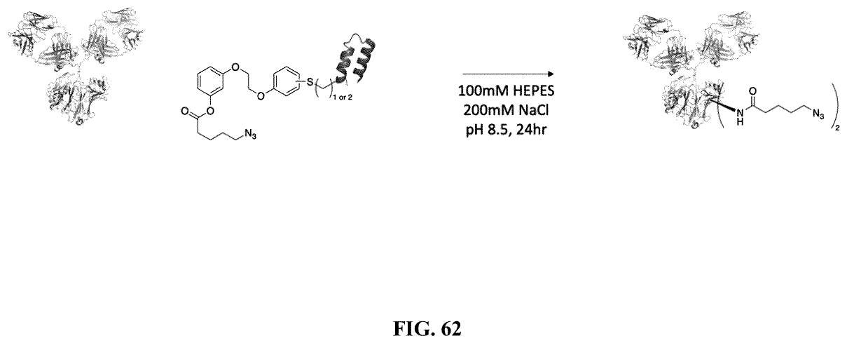

Reactions were performed on a 20 μL scale using 0.2 mL PCR tubes (FIG. 61). First, the electrophile-attached Z33 peptide reagent was dissolved in water to prepare a 2 mg/mL solution. Trastuzumab (MedChemExpress LLC. or Bio X Cell), electrophile-attached Z33 peptide reagent (2 mg/mL), and buffer were added to achieve final concentrations of 10 μM, 200 μM, and values listed in the following table, respectively. If the HEPES was used as a buffer, NaCl solution (4M, 1 uL) was added to the reaction mixture. The contents were mixed using a 10 μL pipette, followed by incubation at room temperature or 37° C. in the dark for 24 hours. The resulting reaction solution was quenched with a 100 mM glycine buffer of 100 mM Tris buffer and analyzed by LC-MS. Sample preparation for LC-MS analysis was performed in the same manner previously described. In entries 12 and 13, slight non-specific modifications were observed during the post-quenching incubation. This suggests that the quenching performed using the mentioned reagents may have been insufficient (Table 6). Therefore, a post-processing step was implemented to remove electrophiles using an Amicon® filter. The reaction mixture was diluted to 200 μL with citrate buffer (100 mM, pH 2.7) and filtered through an Amicon® filter (0.5 mL, 50KMWCO) at 14000 rcf for 7 minutes. After filtration, 400 μL of Tris buffer (100 mM, pH 8.1) was added and centrifuged again. Finally, the remaining solution (roughly 20 μL) on the Amicon® filter was transferred to a microcentrifuge tube, and the Amicon® filter was washed twice with 10 μL of Tris buffer (100 mM, pH 8.1). LC-MS results of electrophile-attached Z33E20C/Hcy reagents are shown in FIG. 51 to FIG. 52E and in Table 7. Unless otherwise provided, “E²⁰C” refers to that the residue of the 20^{th} amino acid of Z33 (which is an E) is replaced with a substituted or unsubstituted residue of cysteine. Unless otherwise provided, “E20Hcy” refers to that the residue of the 20^{th} amino acid of Z33 (which is an E) is replaced with a substituted or unsubstituted residue of homocysteine (“Hcy”).

### Large Scale Reaction Procedure for Entry 6

The reaction was performed on a 2.1 mL scale using a 1.5 mL microcentrifuge tube. First, PEL-21 was dissolved in water to prepare a 2 mg/mL solution. Trastuzumab (Bio X Cell, 9.2 mg/mL, 322 μL), PEL-21 (2 mg/mL, 429 μL), HEPES buffer (160 mM, 1249 μL, pH 8.5), and NaCl solution (4M, 100 μL) were added and mixed using a 200 μL pipette, followed by incubation at room temperature on the shaker for 24 hours. The reaction mixture was diluted to 4 mL with citrate buffer (100 mM, pH 2.7) and filtered through an Amicon® filter (4 mL, 50KMWCO) at 3220 rcf for 10 minutes. After filtration, 2 mL of citrate buffer (100 mM, pH 2.7) was added and centrifuged twice, followed by centrifugation with 2 mL of Tris buffer (50 mM, pH 8.0) three times (total of 6 centrifugations). The remaining solution on the Amicon® filter was transferred to a microcentrifuge tube, and the Amicon® filter was washed twice with 40 μL of Tris buffer (100 mM, pH 8.1). Finally, modified trastuzumab (1.79 mg/mL) was obtained in 75% isolation yield.

TABLE 7
IgG conjugation using electrophile-attached Z33E20C/Hcy reagents analyzed by Q-ToF LC-MS.

| | | | | | | Result analyzed by Q-ToF LC-MS | | |
|---|---|---|---|---|---|---|---|---|
| | | | | | | | | Heavy |
| | | | Equi- | | | Light | | chain total |
| | | | valent | | | chain | Heavy | conversion |
| | PEL | | of | Reaction | Reaction | total | chain total | ratio |
| Entry | # | R | reagent | buffer | Temp. | conversion | conversion | (+1/+2) |
| 1 | 16 | 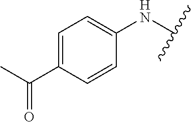 [SMILES: *Nc1ccc(C(C)=O)cc1] | 8 | 20 mM HEPES pH 8.4 | 37 | 36% | 61% | 67/33 |
| 2 | 17 | 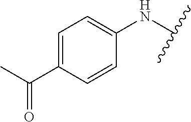 [SMILES: *Nc1ccc(C(C)=O)cc1] | 10 | 100 mM HEPES pH 8.5 | room temperature | 17% | 39% | 82/18 |
| 3 | 18 | 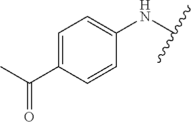 [SMILES: *Nc1ccc(C(C)=O)cc1] | 20 | 100 mM HEPES pH 8.5 | room temperature | 20% | 47% | 77/23 |
| 4 | 19 | 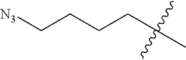 [SMILES: *CCCCN=[N+]=[N-]] | 20 | 100 mM HEPES pH 8.7 | room temperature | 6% | 61% | 90/10 |
| 5 | 20 | 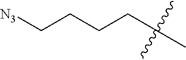 [SMILES: *CCCCN=[N+]=[N-]] | 10 | 100 mM HEPES pH 8.5 | room temperature | 10% | 47% | 100/0 |
| 6 | 21 | 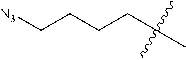 [SMILES: *CCCCN=[N+]=[N-]] | 10 | 100 mM HEPES pH 8.5 | room temperature | 5% | 99% | 100/0 |
| 7 | 21 | 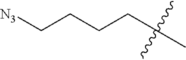 [SMILES: *CCCCN=[N+]=[N-]] | 20 | PBS | room temperature | 12% | 68% | 88/12 |
| 8 | 30 | 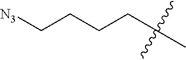 [SMILES: *CCCCN=[N+]=[N-]] | 10 | 100 mM HEPES pH 8.5 | room temperature | 5% | 95% | 95/5 |
| 9 | 30 | 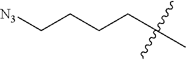 [SMILES: *CCCCN=[N+]=[N-]] | 10 | PBS | 37 | 4% | 86% | 95/5 |
| 10 | 31 | 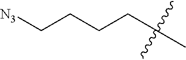 [SMILES: *CCCCN=[N+]=[N-]] | 10 | 100 mM HEPES pH 8.5 | room temperature | <1% | 17% | 100/0 |
| 11 | 31 | 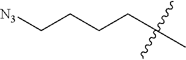 [SMILES: *CCCCN=[N+]=[N-]] | 10 | PBS | room temperature | <1% | 12% | 100/0 |
| 12 | 22 | 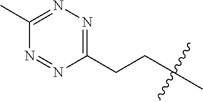 [SMILES: *CCc1nnc(C)nn1] | 10 + 10^{a} | 100 mM HEPES pH 8.7 | 37 | <1% | 93% | 100/0 |
| 13 | 32 | 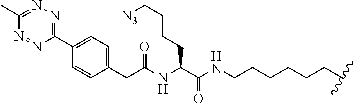 [SMILES: *CCCCCCNC(=O)[C@H](CCCCN=[N+]=[N-])NC(=O)Cc1ccc(-c2nnc(C)nn2)cc1] | 10 + 10^{a} | 100 mM HEPES pH 8.7 | 37 | <1% | 84% | 100/0 |

^{a}10 equivalents of the reagent were added at the beginning and after 4 hours of reaction, respectively.

### Small Bioconjugation Handles Installation to Trastuzumab—Using the Electrophile-Attached Z33M3C/Hcy Reagents

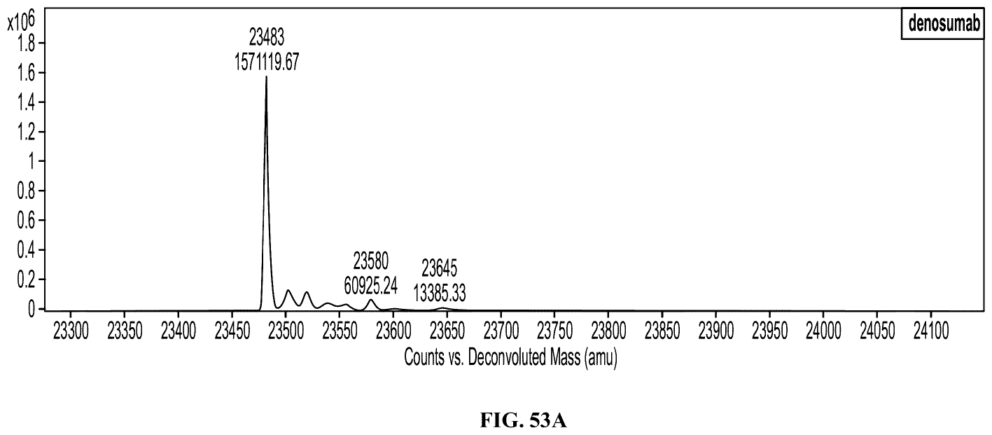

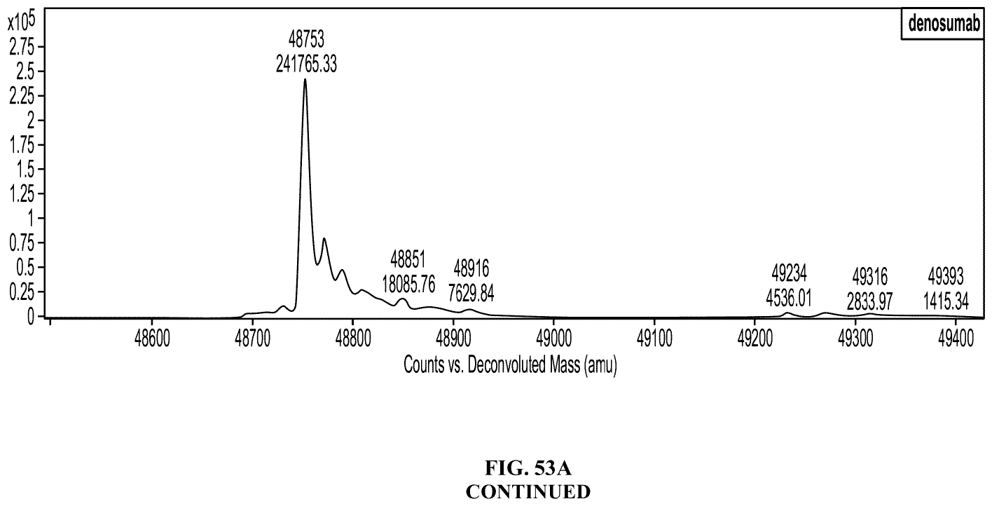

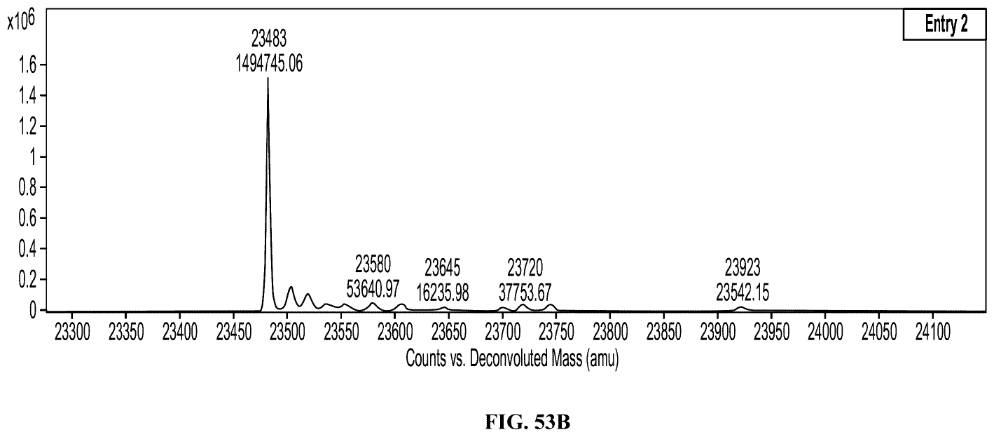

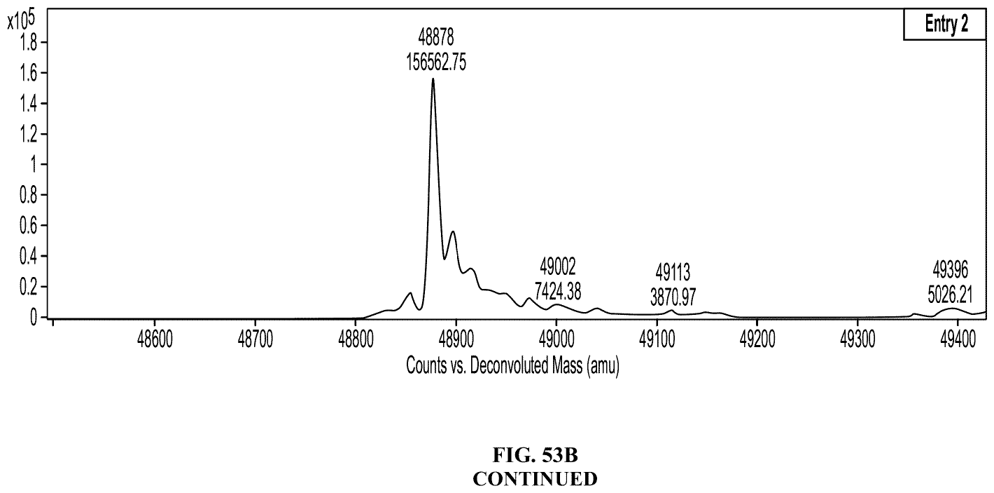

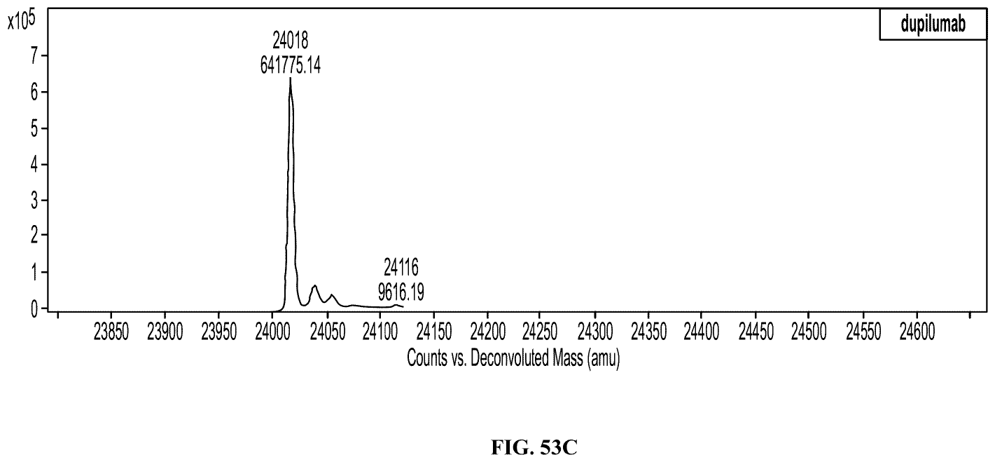

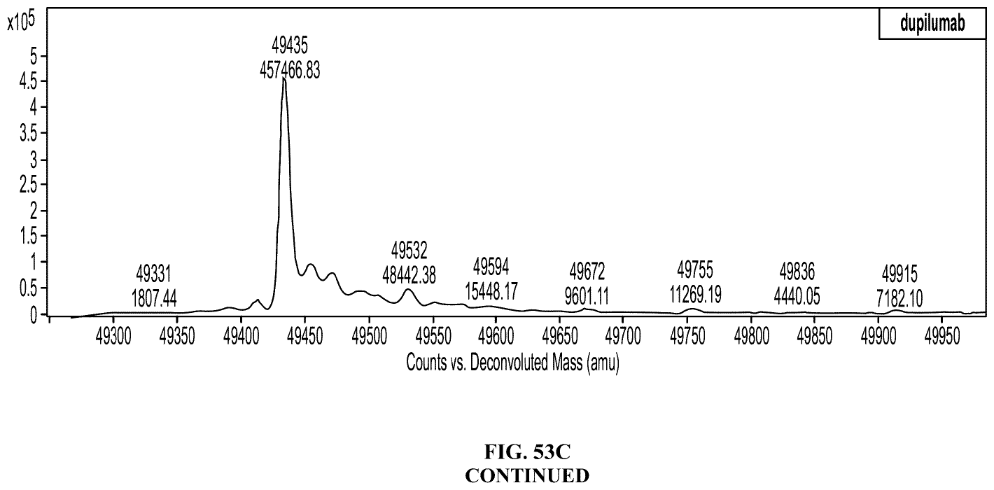

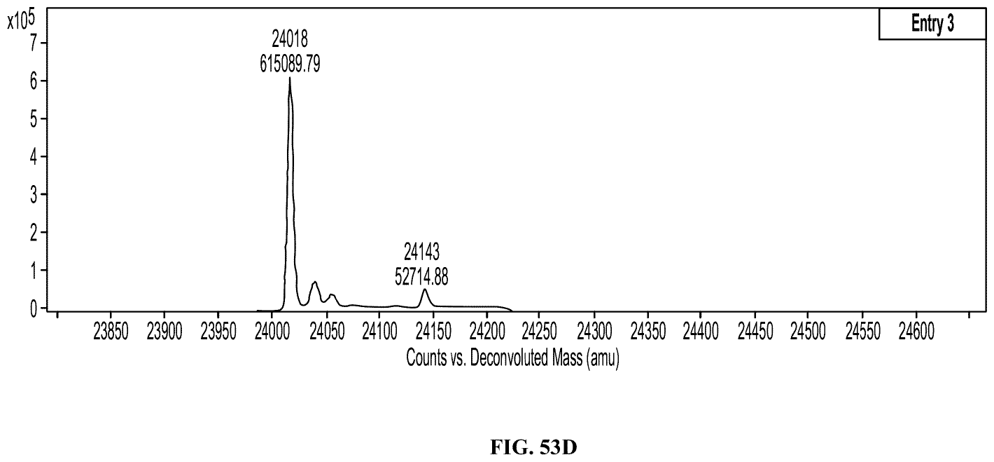

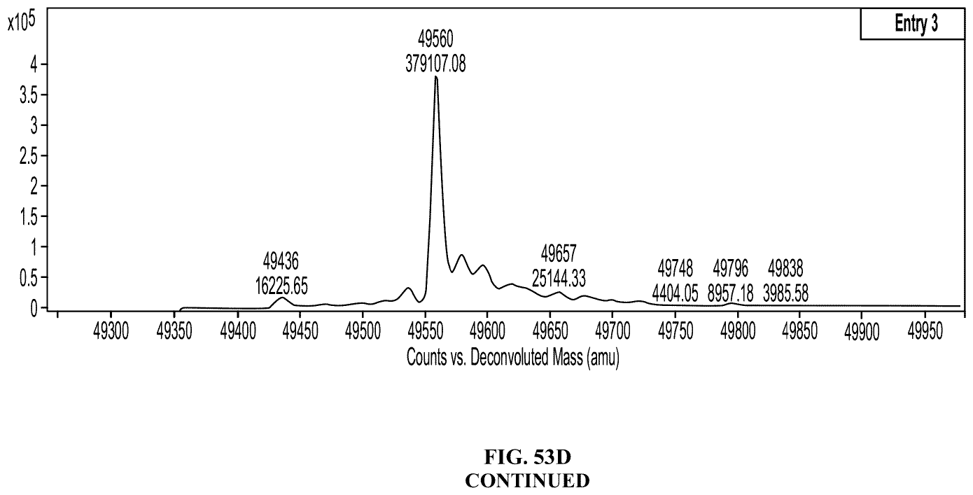

Reactions were performed on a 20 μL scale using 0.2 mL PCR tubes (FIG. 61). First, the electrophile-attached Z33 peptide reagent was dissolved in water to prepare a 2 mg/mL solution. Trastuzumab (Bio X Cell), electrophile-attached Z33 peptide reagent (2 mg/mL), HEPES buffer (pH 8.5), and NaCl solution were added to achieve final concentrations of 10 uM, 200 μM, 100 mM, and 200 mM. The contents were mixed using a 10 μL pipette, followed by incubation at room temperature or 37° C. in the dark for 24 hours. The resulting reaction solution was quenched with a 100 mM Tris buffer and analyzed by LC-MS (FIG. 53, Table 8). Sample preparation for LC-MS analysis was performed in the same manner as previously described.

TABLE 8
IgG conjugation using electrophile-attached
Z33M3C/Hcy reagents analyzed by Q-ToF LC-MS.

| | | | | Result analyzed by Q-ToF LC-MS | | |
|---|---|---|---|---|---|---|
| | | | | | | Heavy |
| | | | | | | chain |
| | | | | Light | Heavy | total |
| | | Equiv- | | chain | chain | conversion |
| | PEL | alent of | Reaction | total | total | ratio |
| Entry | # | reagent | Temp. | conversion | conversion | (+1/+2) |
| 1 | 23 | 10 | room | <1% | 44% | 100/0 |
| | | | temperature | | | |
| 2 | 24 | 20 | room | <1% | 27% | 82/18 |
| | | | temperature | | | |
| 3 | 25 | 10 | 37° C. | <1% | 99% | 85/15 |
| 4 | 26 | 20 | room | <1% | 38% | 88/12 |
| | | | temperature | | | |

### Small Bioconjugation Hands Installation to Trasuzumab—Using the Electrophile-Attached Z33R31C/Hcy and D32C/Hcy Reagents

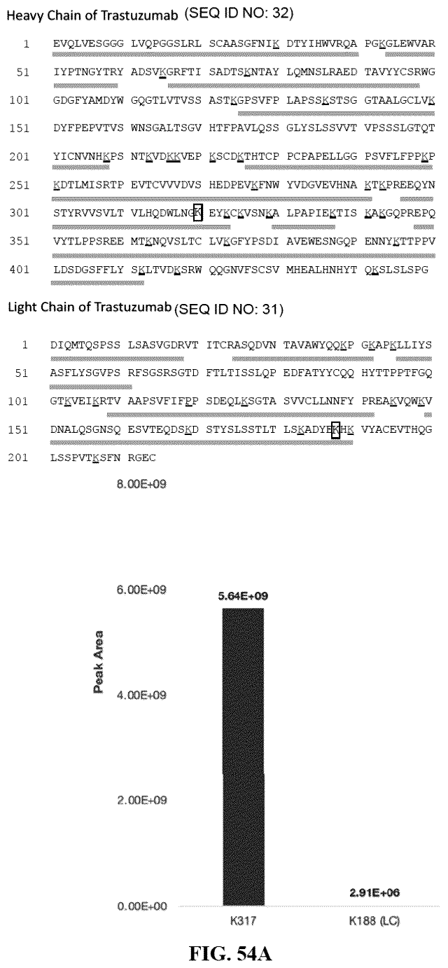

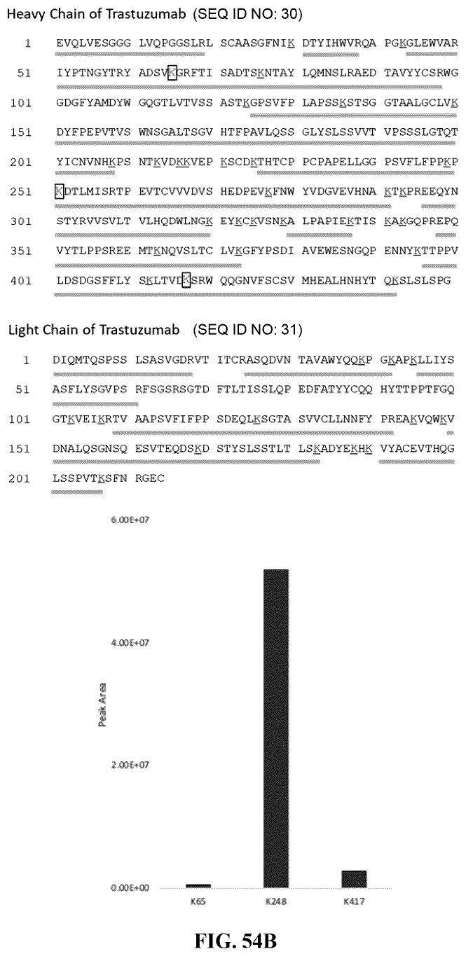

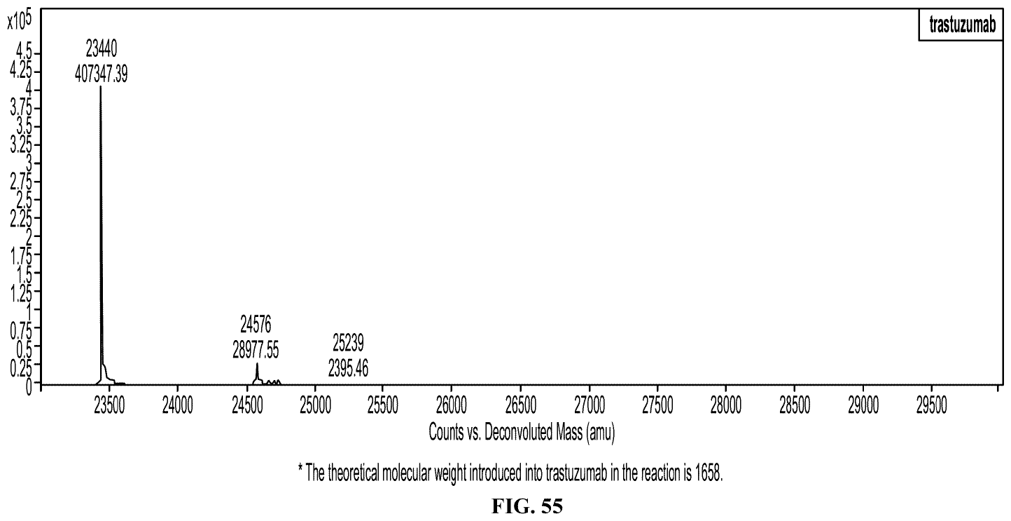

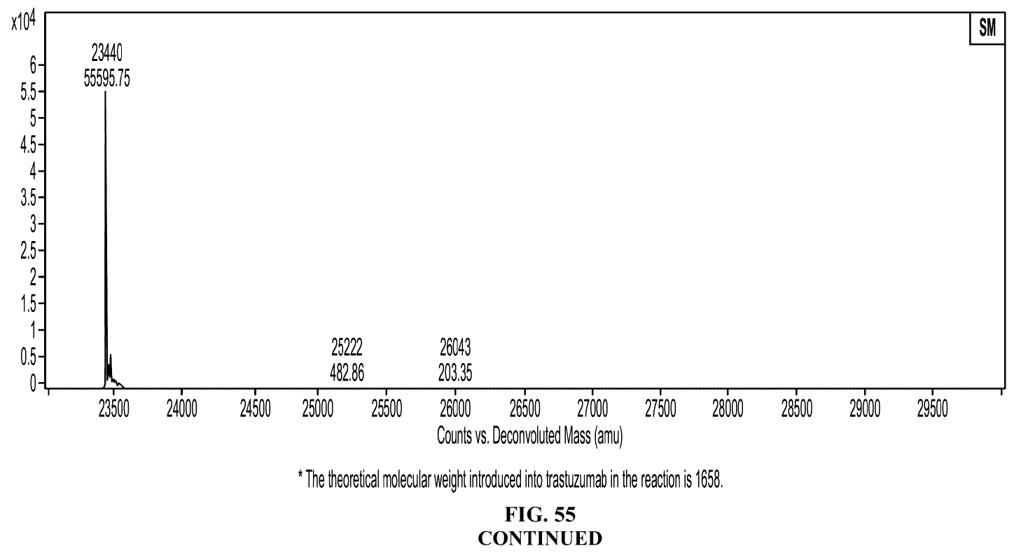

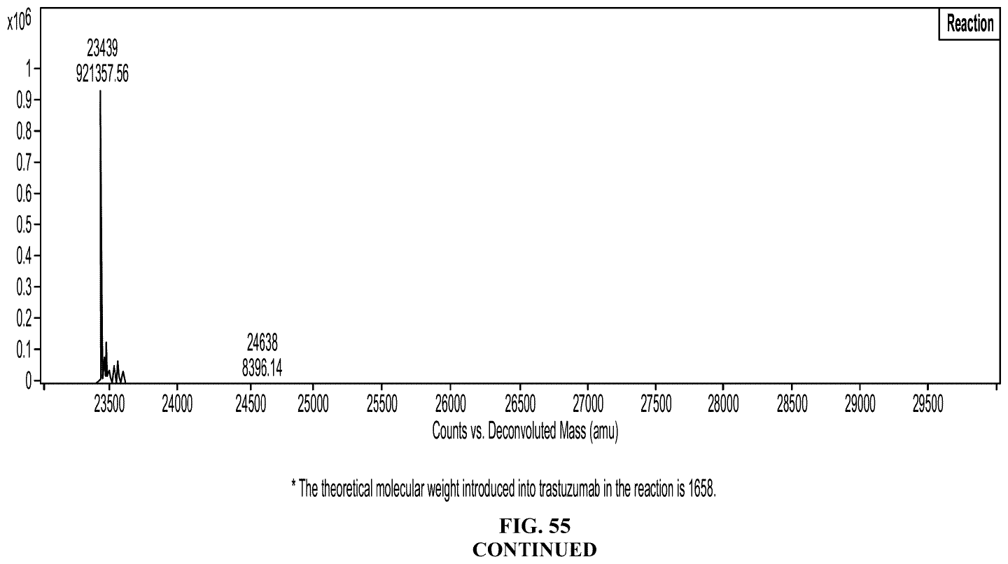

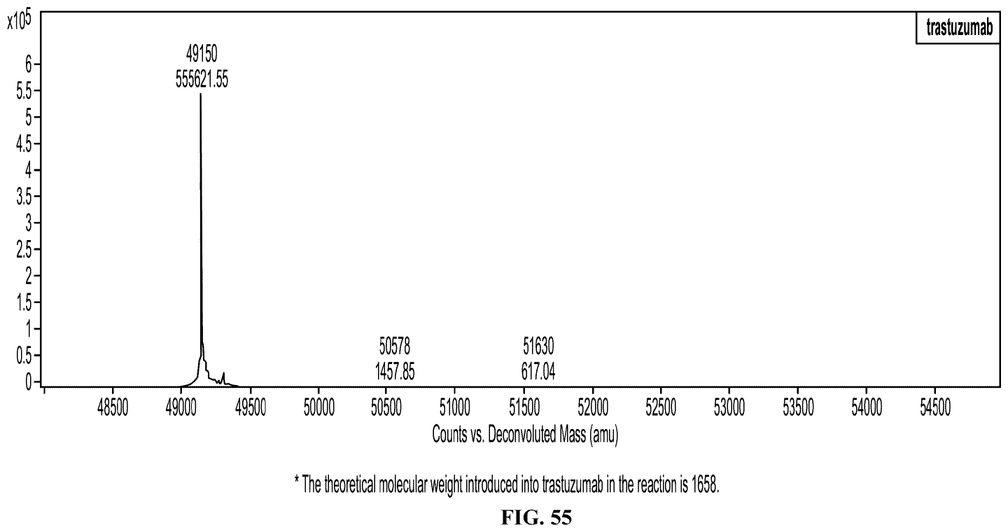

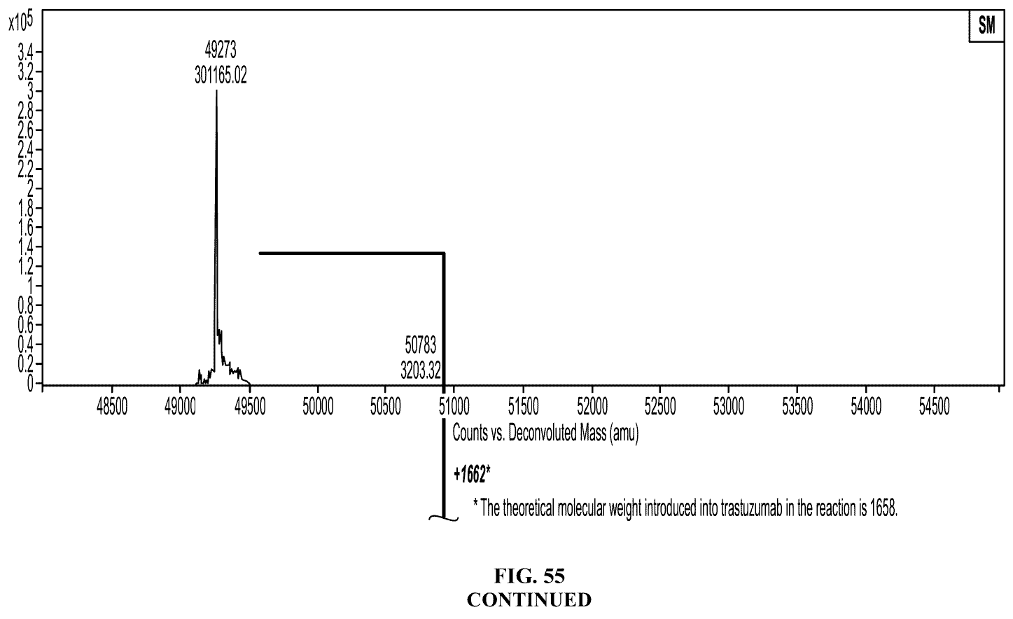

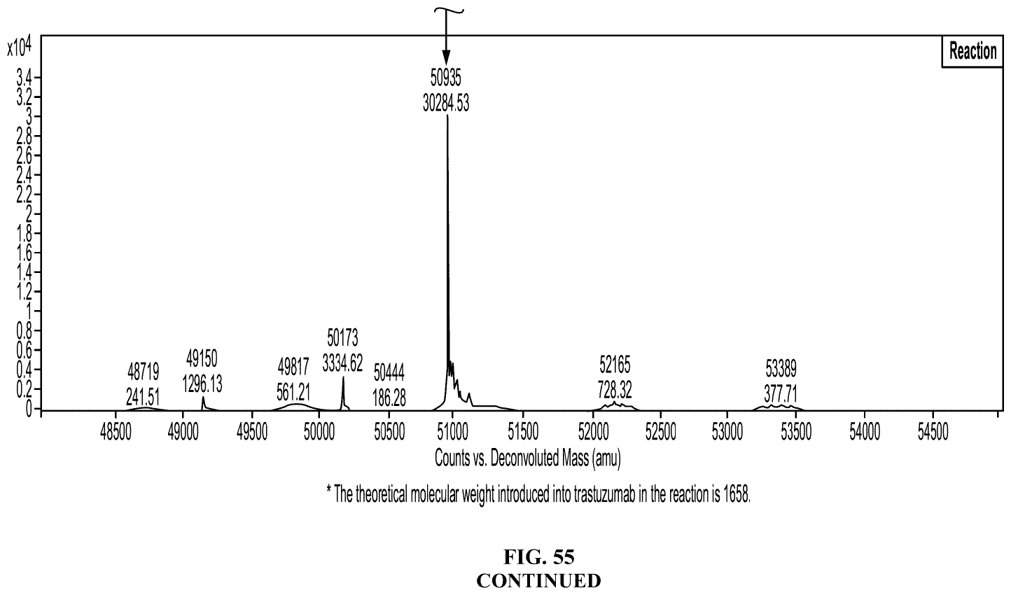

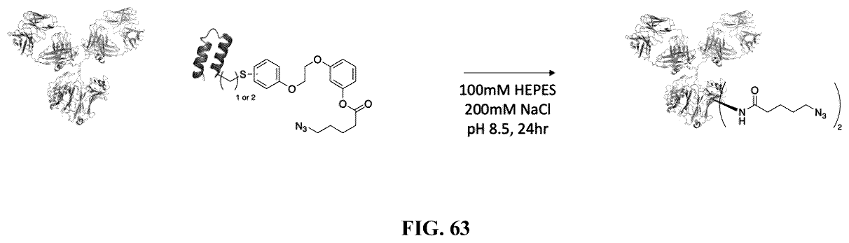

Reactions were performed on a 20 μL scale using 0.2 mL PCR tubes (FIG. 63). First, the electrophile-attached Z33 peptide reagent was dissolved in water to prepare a 2 mg/mL solution. Trastuzumab (Bio X Cell), electrophile-attached Z33 peptide reagent (2 mg/mL), HEPES buffer (pH 8.5), and NaCl solution were added to achieve final concentrations of 10 uM, 200 μM, 100 mM, and 200 mM. The contents were mixed using a 10 μL pipette, followed by incubation at room temperature or 37° C. in the dark for 24 hours. The resulting reaction solution was quenched with a 100 mM Tris buffer and analyzed by LC-MS (FIG. 54, Table 9). Sample preparation for LC-MS analysis was performed in the same manner as previously described.

TABLE 9
IgG conjugation using electrophile-attached Z33R31C/Hcy
and D32C/Hcy reagents analyzed by Q-ToF LC-MS.

| | | | | Result analyzed by Q-ToF LC-MS | | |
|---|---|---|---|---|---|---|
| | | | | | | Heavy |
| | | | | | | chain |
| | | | | Light | Heavy | total |
| | | Equiv- | | chain | chain | conversion |
| | PEL | alent of | Reaction | total | total | ratio |
| Entry | # | reagent | Temp. | conversion | conversion | (+1/+2) |
| 1 | 27 | 10 | room | <1% | 21% | 100/0 |
| | | | temperature | | | |
| 2 | 28 | 20 | room | <1% | 32% | 100/0 |
| | | | temperature | | | |
| 3 | 29 | 10 | 37° C. | <1% | 27% | 100/0 |

### Small Bioconjugation Handles Installation to Other IgGs

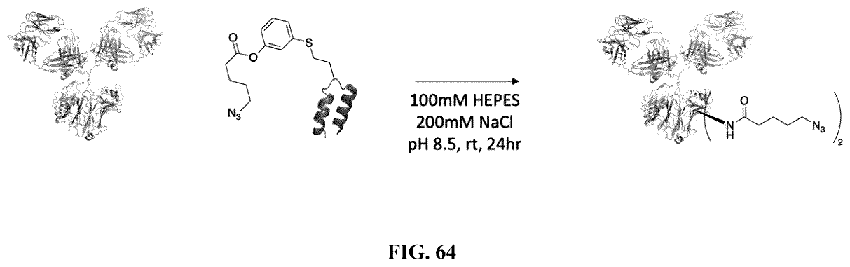

Reactions were performed on a 10 μL scale using 0.2 mL PCR tubes (FIG. 64). First, the electrophile-attached Z33 peptide reagent was dissolved in water to prepare a 2 mg/mL solution. IgG (human WN1 (prepared as previously described (ChemBioChem, 19, 2039-2044 (2018)), denosumab (Invitrogen), dupilumab (Invitrogen), or mouse IgG1 (Invitrogen, subtype controlled)), electrophile-attached Z33 peptide reagent (2 mg/mL), HEPES buffer (pH 8.5), and NaCl solution were added to achieve final concentrations of 5 μM, 100 μM, 100 mM, and 200 mM. The contents were mixed using a 5 μL pipette and incubated at room temperature in the dark for 24 hours. The resulting reaction solution was quenched with a 100 mM Tris buffer and analyzed by LC-MS (FIGS. 53A to 53D, Table 10). Sample preparation for LC-MS analysis was performed in the same manner as previously described.

TABLE 10
Conjugation of electrophile-attached Z33 reagent
to other IgGs analyzed by Q-ToF LC-MS.

| | | | | | Result analyzed by Q-ToF LC-MS | | |
|---|---|---|---|---|---|---|---|
| | | | | | Light | Light | Light |
| | | PEL | Equivalent | Reaction | chain total | chain total | chain total |
| Entry | IgG | # | of reagent | temp. | conversion | conversion | conversion |
| 1 | human WN1 | 21 | 20 | room | 2% | 99%< | 100/0 |
| | | | | temperature | | | |
| 2 | Denosumab | 21 | 20 | room | 1% | 99%< | 100/0 |
| | (hIgG2) | | | temperature | | | |
| 3 | Dupilumab | 21 | 20 | room | 13% | 96% | 100/0 |
| | (hIgG4) | | | temperature | | | |
| 4 | Mouse IgG1 | 21 | 20 | room | 2% | 69% | 100/0 |
| | | | | temperature | | | |

### Determination of the Modification Sites

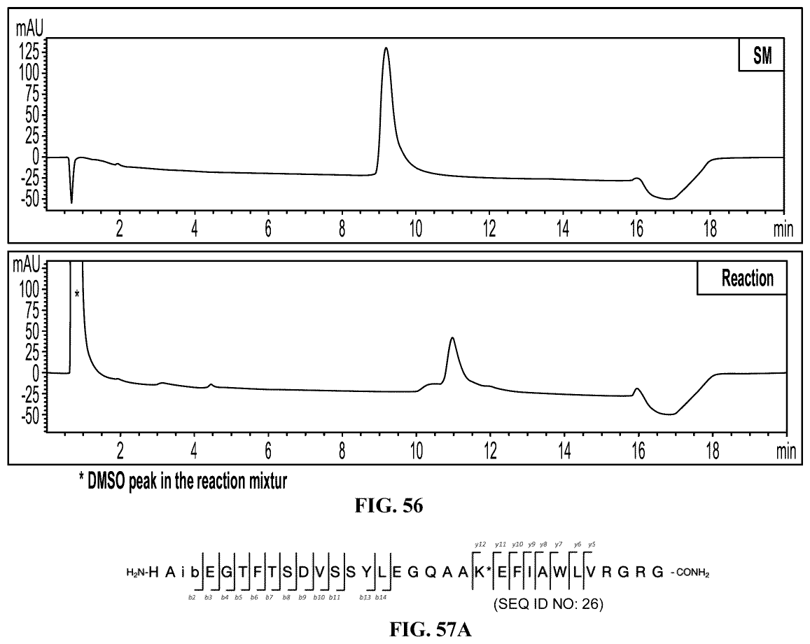

The modified trastuzumab (30 μg) from entry 6 of 5.2.1 was diluted to 36 μL using Tris buffer (50 mM, pH 8.0). PNGase F (New England BioLabs, 200 units) was added to the solution and incubated at 37° C. for 4 hours. The reaction solution was then brought to room temperature, and urea solution (6M in 50 mM Tris pH 8.0, 14 μL) and DTT solution (200 mM, 1.5 μL) were added and incubated at 37° C. for 1 hour. After that, iodoacetamide (800 mM, 1 μL) was added and incubated at room temperature for 30 minutes. The resulting solution was diluted 2-fold using Tris buffer (50 mM, pH 8.0), and then Trypsin/Lys-C mix (Promega, 0.2 μg/μL in solution, 6 μL) was added and incubated at 37° C. for 18 hours. The reaction solution was then purified by pipetting with Ziptip, lyophilized, and analyzed by nLC-MS/MS (using nLC-MS/MS method B). Obtained raw data were analyzed using Thermo Scientific FreeStyle™ 1.6. De novo sequencing was performed with PEAKS 8.5 with the following search parameters: Parent Mass Error Tolerance=15.0 ppm; Fragment Mass Error Tolerance=0.02 Da, Enzyme=None; modification setting=table below; Max Variable PTM Per Peptide=10; Report #Peptides=10. Sequencing results were exported as .csv reports for all de novo candidates. Finally, fragments corresponding to trastuzumab sequences were manually searched from the .csv file, and the main modification sites were estimated using each fragment's peak area (Table 11). Since the mass of the modified lysine is the same as the mass of Pro-Arg (271.1644), both possibilities were considered in the fragment search (FIGS. 56A to 56B).

TABLE 11
Modification sites estimated using each fragment's peak area.

| | Amino | Molecular | |
|---|---|---|---|
| PTM type | acids | weight | Setting Reason |
| Fixed | C | 57.0214 | Capped by iodoacetamide |
| Variable | K | 125.0589 | Modified by the electrophile |
| | | | attached peptide reagents |
| variable | M | 15.9949 | Unintentional natural oxidation |

### Example 7. Antibody-Drug Conjugate (ADC) Preparation

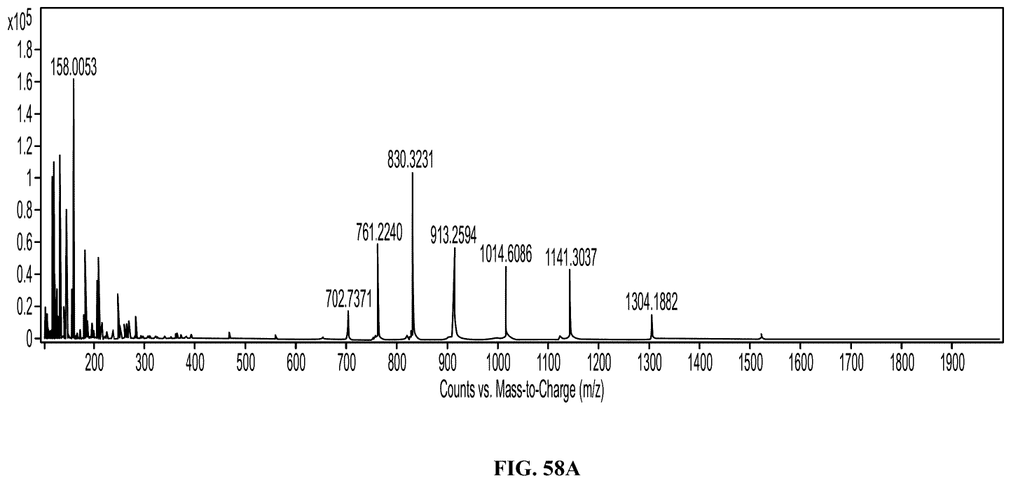

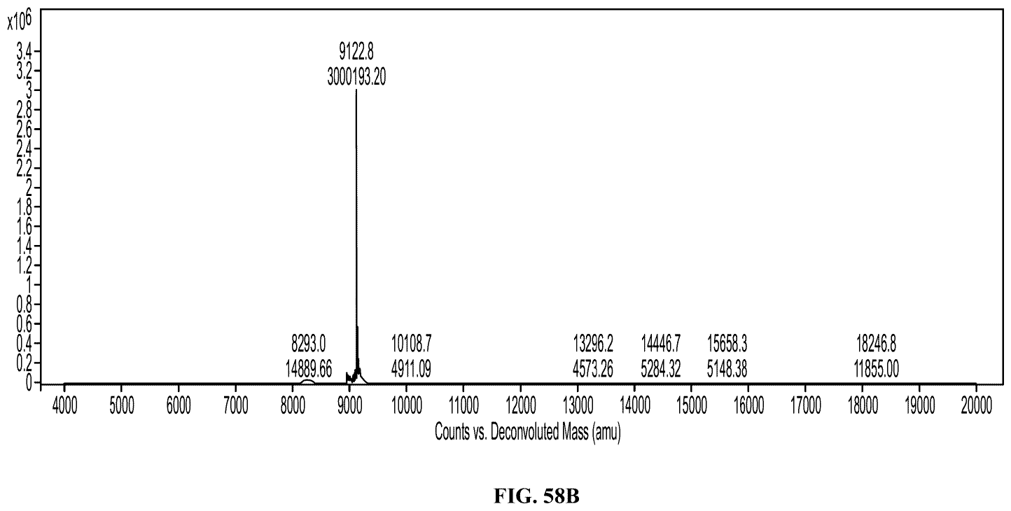

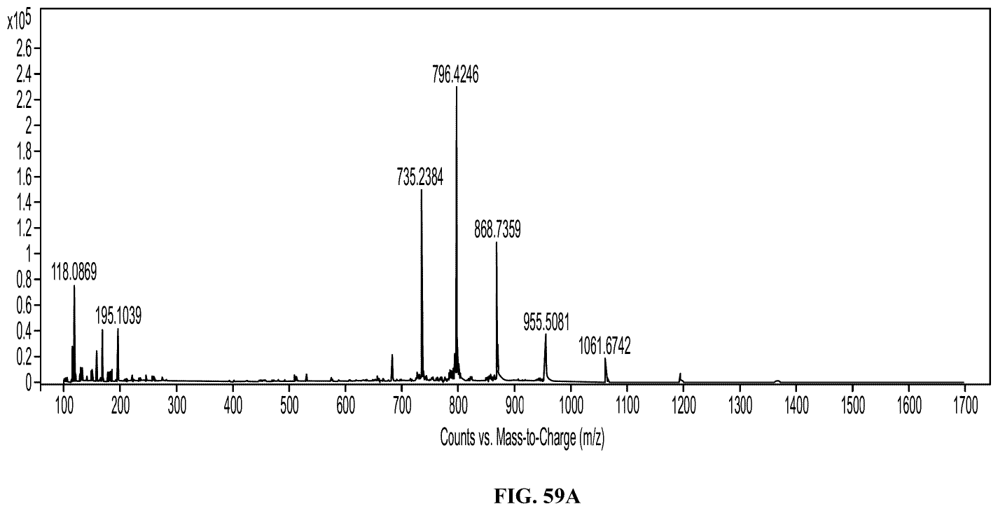

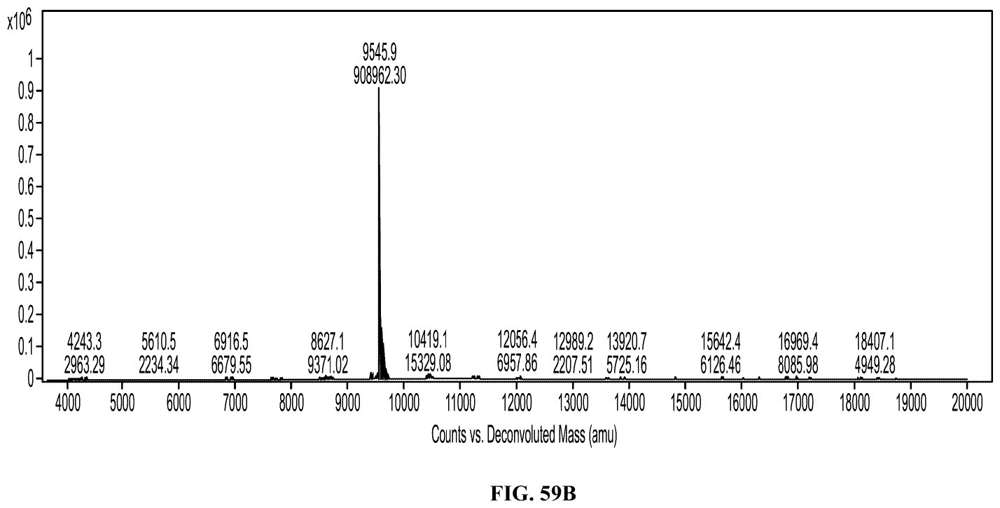

Reactions were performed on a 10 μL scale using 0.2 mL PCR tubes. Modified trastuzumab (7.26 μg) obtained from entry 6 of 5.2.1 was mixed with DBCO-PEG4-VC-PAB-MMAE (MedChemExpress, 50 mM in DMSO), Tris buffer (pH 8.0), and NaCl solution to achieve final concentrations of 5 uM, 100 μM, 50 mM, and 200 mM, respectively. The reaction mixture was then incubated at room temperature for 24 hours. After incubation, the reaction solution was divided into two portions: 2 μL for analysis by LC-MS and 8 μL for analysis by HPLC. The 2 μL sample was prepared for LC-MS analysis in a similar manner as described in the “Mouse IgG Painting with Azido Moieties” section. The 8 μL sample was diluted to 40 μL using a 95:5 water-acetonitrile solution (+0.1% TFA), and 20 μL of this diluted sample was injected into the HPLC system for analysis (FIG. 58 and FIG. 59).

### Example 8. Mouse IgG Painting with Azido Moieties

### Enzyme-Linked Immunosorbent Assay (ELISA)

Two sandwich ELISA assays have been developed in-house, one for the detection of the azido moiety conjugated mouse IgG (mIgG, E1), the other one for the detection of the whole mIgGs (modified and unmodified) in the sample (E2). Both E1 and E2 were run simultaneously on a same plate, using the same initial stock sample, and were processed in parallel at the same time for every steps. The same azido-free capture goat anti-mouse polyclonal antibody (Creative Diagnostics) was used for both E1-E2 assays at 1 μg/mL (100 μL per well) for incubation overnight at 4° C. Washing steps were performed thoroughly between each incubation by using 0.02% Tween 20-PBS. Saturation step (1 h, ROOM TEMPERATURE) and sample dilutions were all carried in 5% milk-PBS. For E1, the detection was performed using 1:150 dilution of DBCO-PEG4-Biotin (Jena Bioscience) (in 5% milk-PBS) while for E2 the detection was made using 1:2000 azido-free biotin-conjugated donkey anti-mouse polyclonal antibody (Creative Diagnostics) in 5% milk-PBS, both for 150 minutes at ROOM TEMPERATURE, in the dark. A dilution of 1:500 HRP-conjugated streptavidin (Thermo Fisher Scientific Inc) in 5% milk-PBS was then added for 1 h, ROOM TEMPERATURE, in the dark. TMB substrate (Thermo Fisher Scientific Inc) was added (100 μL per well, ROOM TEMPERATURE) simultaneously in E1 and E2 wells, and the reaction was stopped with 2N H2SO4 stop solution (Thermo Fisher Scientific Inc) after 5 to 10 minutes. Absorbances were measured at 450 nm and 540 nm (A0=A450 nm-A540 nm) using a Spark microplate reader (Tecan, USA). The background noise (Ablank) from the serum was subtracted to get the final absorbance of the sample (Asample=A0-Ablank). The ratio Asample (E1)/Asample (E2) was calculated to estimate the percentage of azido transfer on the total amount of IgG in the sample.

### Qualitative Assessment of Azido-Transfer to Mouse IgG in Serum

Reactions were performed on a 40 μL scale using 0.2 mL PCR tubes. First, the electrophile-attached Z33 peptide reagent PEL-21, PEL-30 or PEL-31 was dissolved in water to prepare a 2 mg/mL solution. Mouse serum (Invitrogen, S/N 24-5544-94, 31.2 μL) and electrophile-attached Z33 peptide reagent (2 mg/mL, 8.8 μL) were added to the PCR tubes to a final concentration of 100 mM of electrophile-attached Z33 reagent. The contents were mixed using a 10 μL pipette, followed by incubation at 37° C. in the dark for 2, 6, or 24 hours. To remove the excess amount of the remaining electrophile-attached Z33 reagent, the reaction mixture was filtered using an Amicon® filter. The reaction mixture was diluted to 200 μL with citrate buffer (100 mM, pH 2.7) and filtered through an Amicon® filter (0.5 mL, 30KMWCO) at rcf for 7 minutes. After filtration, 400 μL of PBS buffer was added and centrifuged again. Finally, the remaining solution (roughly 20 μL) on the Amicon® filter was transferred to a microcentrifuge tube. Obtained samples were then diluted 1:100 to 1:2000 in 5% milk-PBS for dosage using E1/E2 assays.

### Example 9. Mouse IgG Painting with Radionuclides

Preparation of Deferoxamine B-Attached Z33 Reagents and Radiolabeling with Zirconium-89

First, PEL-21 or PEL-31 were dissolved in HEPES buffer (100 mM in ultra-trace elemental water; Fischer scientific, pH 6.7) to prepare a 2 mg/mL solution of each. Deferoxamine-DBCO (Macrocycles™, 1 mM in DMSO, 11.1 μL) and PEL-21 or PEL-31 (360 μL) solutions were then added to the tube, stirred gently by hand, and the tube was placed on ice for 1 hour.

The obtained samples were incubated with 37 MBq of [⁸⁹Zr]Zr-oxalate (dilution in 0.5M HEPES, pH 6.7) for 1 hour in the wet ice under mild agitation. Purification was then performed using a Zeba spin column (0.5 mL, 7KMWCO) to remove the non-complexed [⁸⁹Zr]Zr-oxalate, for 5 min at 1,200 rcf. Instant-layer thin chromatography (iTLC) was performed to verify the success of the radiolabeling with elution in 0.1 M citrate buffer.

Pharmacokinetic of Radionuclide Transfer to mIgGs and Whole-Body Biodistribution

For injection in animals, samples were incubated with 55.5 MBq of [⁸⁹Zr]Zr-oxalate (dilution in 0.5M HEPES, pH 6.7) for 1.5 hours in wet ice under mild agitation. No purification step was performed as a previous iTLC analysis showed no big purity difference between the pre and post purification of samples.

Swiss mice (n=4 per group) were IP injected with 1.2 to 1.5 MBq of [⁸⁹Zr]Zr-PEL-21 or [⁸⁹Zr]Zr-PEL-31. After 2 h, 24 h, 72 h, and 144 hours post injection, the mice were sacrificed to collect the following fluids and organs: blood, urine, feces, heart, lungs, liver, spleen, stomach, small intestines, colon, kidneys, bladder, pancreas, caecum, uterus+ovaries, gastrocnemius muscle, bone, skin, brain, and eyes. Each sample was weighted and count in a gamma counter (Wizard2, Perkin Elmer LLC, USA) to get the counts per minutes (cpm) for determining the percentage of injected activity per gram (% IA/g) in each organ. Cpm were decay-corrected by using the following formula: A=A₀ e((−(ln2)Δt)/T) with A=decay-corrected cpm, A₀=raw cpm, Δt=time difference between the injection and the cpm measurement, and T=half-life of the radionuclide (i.e. 78.41 h for Zr-89).

### Example 10. Mouse IgG Painting with Bioactive GLP-1 Peptide Analogs

Preparation of the GLP1 analog-attached Z33 transfer reagents

GLP1 analog GLP-01 was synthesized according to the previously described method, except for the following conditions: ChemMatrix® H-Rink Amide resin (0.19 mmol/g, 150 mg) was used, and the noncanonical amino acid Fmoc-Aib-OH was prepared as a 0.40 M DMF solution and incorporated into the sequence by AFPS. For the introduction of Aib and N-terminal H, the condensation reagent was changed to PyAOP, and pump stroke number was changed to 35. A mixture of TFA, water, thioanisole, phenol, and TIPS in a ratio of 33:2:2:1 was used as the cleavage solution from the resin. Additionally, if CO₂ adducts were observed in the crude product, the crude product was dissolved in a 95:5 water-acetonitrile solution (5 mL), and 200 μL of acetic acid was added before purification. The mixture was incubated at 37° C. for 1 hour and then loaded into a Biotage for purification (Table 12).

TABLE 12
Characteristics of GLP analog GLP-01.

| | N- | | C- | Calculated | Observed |
|---|---|---|---|---|---|
| Code | term | Sequence | term | Mass (Da)^{a} | Mass (Da) |
| GLP- | Free | HAibEGTFTSD | Amide | 3394.7 | 1132.9 |
| 01 | | VSSYLEGQAAK | | | (+3H⁺) |
| | | EFIAWLVRGRG | | | 850.2 |
| | | (SEQ ID | | | (+4H⁺) |
| | | NO: 25) | | | 680.4 |
| | | | | | (+5H⁺) |

^{a}monoisotopic mass

### Synthesis of GLP-03

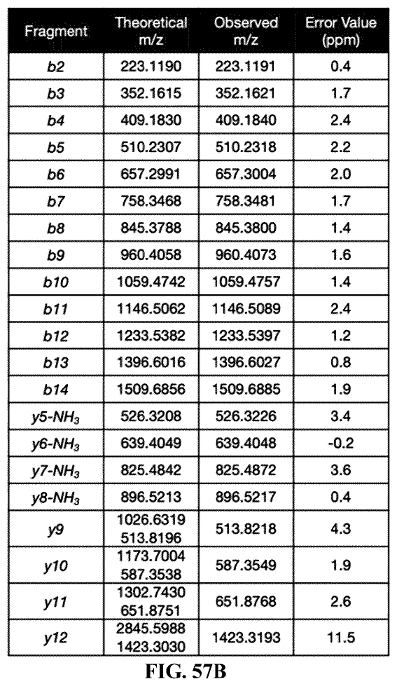

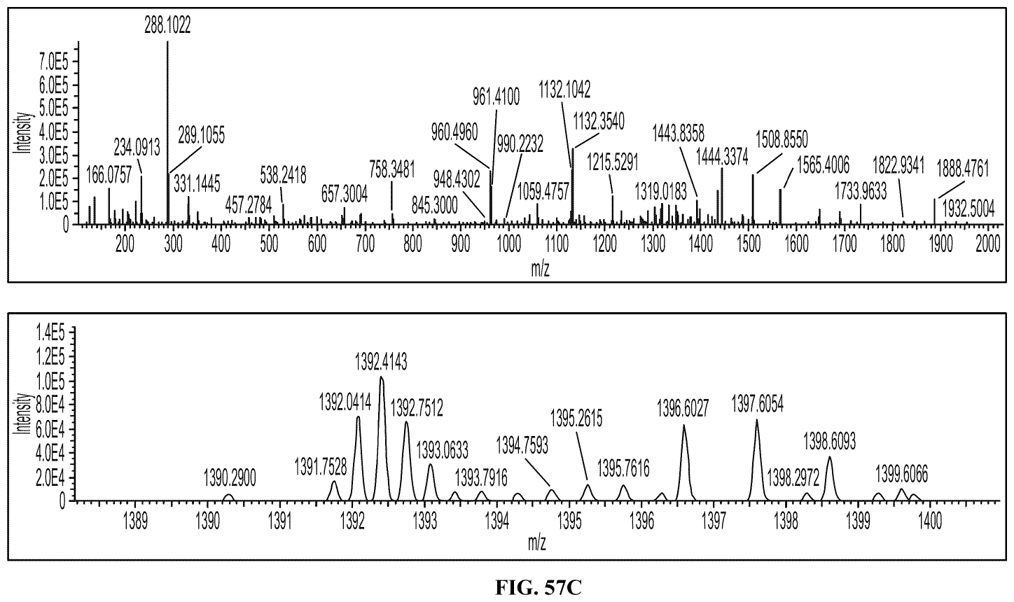

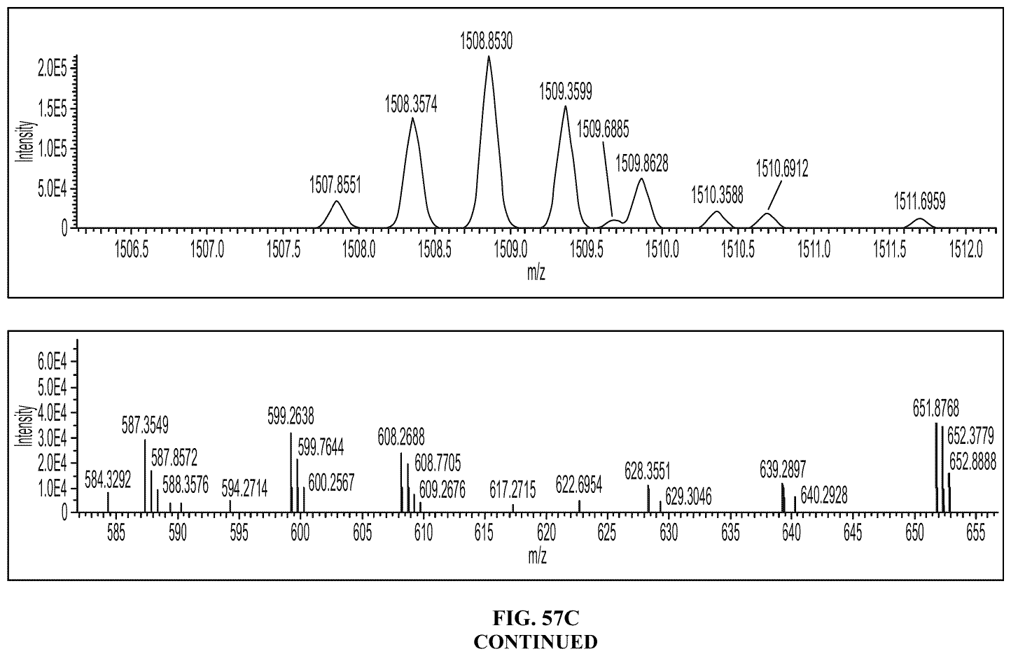

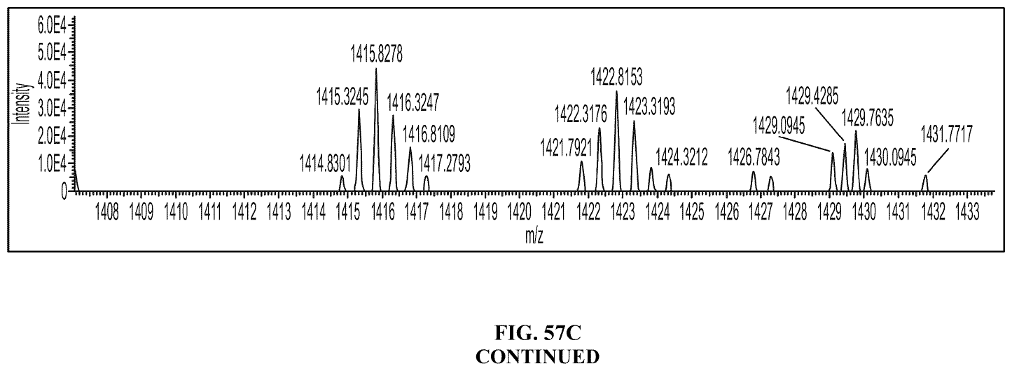

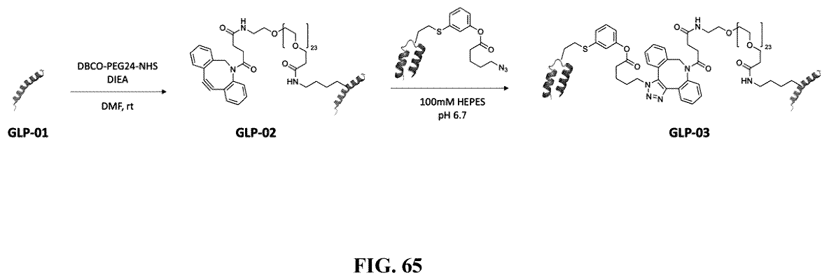

The reaction was performed using a 1.5 mL microcentrifuge tube (FIG. 65). The GLP-01 (18.25 mg) was dissolved in 600 μL of DMF. After adding 9 μL of DIEA, a separately prepared solution of DBCO-PEG24-NHS (BROAD PHARMA®, 30 mg/mL in DMF, 300 μL) was added. The mixture was incubated at room temperature for 30 minutes, and the reaction was quenched with 1-naphthylmethylamine (9 μL). Then the reaction mixture was transferred to a 50 mL centrifuge tube and diluted to 5 mL with water. Subsequently, the reaction solution was directly loaded onto a Biotage® Sfär C18 D column (12 g) and purified using the following conditions: 5CV 5% B, 1 CV 5-35% B, 6 CV 35% B, and 32 CV 35-50% B. 1 μL of a 5-fold diluted solution of the obtained fractions was analyzed by LC-MS (FIGS. 57A to 57C), and the fractions containing only the target product were collected and lyophilized to obtain GLP-02 (9.56 mg) as a white solid.

The modification site of GLP-02 was inferred from the measurement results using nLC-MS/MS (method A). GLP-02 (100 nmol, 1 μL) was injected into the nLC-MS/MS system, and nLC-MS/MS method B was used for this experiment (Table 13).

TABLE 13
Characteristics of GLP analog GLP-02.

| | N- | | C- | Calculated | Observed |
|---|---|---|---|---|---|
| Code | term | Sequence | term | Massª | Mass^{b} |
| GLP- | Free | HAibEGTFTSDV | Amide | 4812.4 | 4812.9 |
| 02 | | SSYLEGQAAK* | | | |
| | | EFIAWLVRGRG | | | |
| | | (SEQ ID NO: 26) | | | |

^{a}average mass, ^{b}after deconvolution, *modified lysine

The reaction was performed using a 15 mL centrifuge tube. First, PEL-21 (4 mg) and GLP-(4.9 mg) were dissolved in HEPES buffer (100 mM, pH 6.7) to prepare a 2 mg/mL solution of each. The PEL-21 and GLP-02 solutions were then added to the centrifuge tube, stirred gently by hand, and the tube was placed on ice for 30 minutes. Then, the reaction solution was directly loaded onto a Biotage® Sfär C18 D column (12 g) and purified using the following conditions: 5 CV 5% B, 1CV 5-20% B, 25CV 20-60% B. 1 μL of a 5-fold diluted solution of the obtained fractions was analyzed by LC-MS, and the fractions containing only the target product were collected and lyophilized to obtain GLP-03 (6.22 mg) as a white solid (FIGS. 58A to 58B). The observed mass after deconvolution was 9123. The calculated average mass was 9122.

### Synthesis of GLP-04

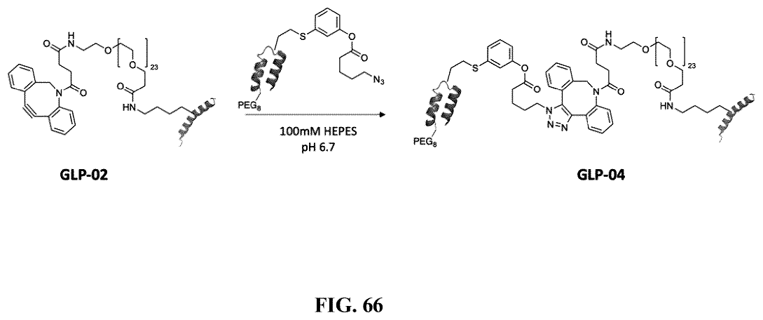

PEL-30 (7.0 mg) and GLP-02 (7.8 mg) were used to synthesize GLP-04 following the same procedure as GLP-03 (FIG. 66). GLP-04 (10.2 mg) was obtained as a white solid. The observed mass after deconvolution was 9545.9 (FIGS. 59A to 59B). The calculated average mass was 9545.4.

### Assessment of GLP1 Transfer Using Human IgG1 Trastuzumab

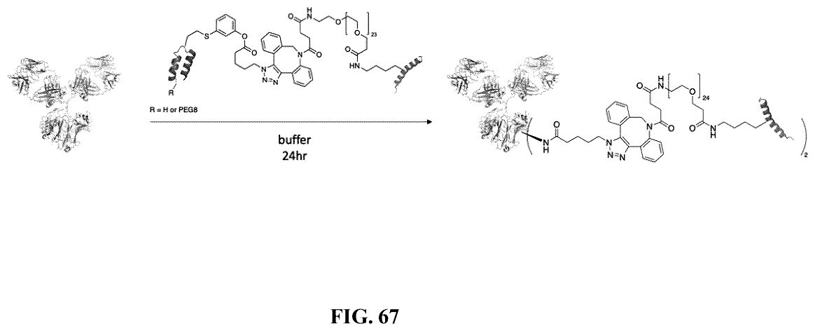

Reactions were performed on a 20 μL scale using 1.5 mL microcentrifuge tubes (FIG. 67). First, GLP-03 or GLP-04 was dissolved in water to prepare a 2 mg/mL solution. A mixture of HEPES buffer and NaCl solution or PBS was added to the tubes, followed by trastuzumab (Bio X Cell, 9.2 mg/mL, 1.61 μL), GLP-03 (2 mg/mL, 9.12 μL) or GLP-04 (2 mg/mL, 9.55 μL) were added, mixed using a 10 μL pipette, and incubated at room temperature or 37° C. for 24 hours. The final concentrations of HEPES buffer, NaCl solution, and PBS buffer were 100 mM, 200 mM, and 1×, respectively. The reaction mixture was then diluted to 200 μL with citrate buffer (100 mM, pH 2.7) and filtered through an Amicon® filter (0.5 mL, 50KMWCO) at 14000 rcf for 7 minutes. After filtration, 400 μL of Tris buffer (100 mM, pH 8.1) was added and centrifuged again. Finally, the remaining solution (roughly 20 μL) on the Amicon® filter was transferred to a microcentrifuge tube, and the Amicon® filter was washed twice with 10 μL of Tris buffer (100 mM, pH 8.1). Sample preparation for LC-MS analysis was performed in the same manner as previously described (Table 14).

TABLE 14
Conjugation of GLP1 analog-attached Z33 transfer
reagents to human IgG₁ analyzed by Q-ToF LC-MS.

| | | | | | Result analyzed by Q-ToF LC-MS | | |
|---|---|---|---|---|---|---|---|
| | | | | | Light | Heavy | Heavy chain |
| | GLP | Equivalent | | Reaction | chain total | chain total | total conversion |
| Entry | # | of reagent | Buffer | Temp. | conversion | conversion | ratio (+1/+2) |
| 1 | 3 | 20 | HEPES | room | <1% | 27% | 100/0 |
| | | | | temperature | | | |
| 2 | 3 | 20 | HEPES | 37° C. | <1% | 35% | 94/6 |
| 3 | 4 | 20 | HEPES | room | <1% | 26% | 100/0 |
| | | | | temperature | | | |
| 4 | 4 | 20 | HEPES | 37° C. | <1% | 28% | 100/0 |
| 5 | 4 | 20 | PBS | 37° C. | <1% | 16% | 100/0 |

Pharmacodynamic Studies of Native IgG Painting with GLP1 Analogs in Living Mice

Swiss mice were SC injected, with either PBS (n=5), or with 10 mg/kg of semaglutide Ozempic® (Adipogen Inc.) (n=15), GLP-01 (n=15), GLP-03 (n=15), or GLP-04 (n=15). Body weight was monitored over 21 days for the entire cohort. At 24 h, 72 h, or 144 hours post injection, an IP-glucose tolerance test (Ip-GTT) was performed on the mice. Six hours before the Ip-GTT, the mice were fasted by removing the food, the enrichment, and by placing them in a new cage to avoid coprophagy. Access to water was, however kept free and ad libitum for the entire experiment. Five minutes before the Ip-GTT, basal glycemia was measured by taking a small drop of blood using tail pricks. Then, 2 g/kg of a 20% solution of dextrose (Sigma Aldrich Inc.) was IP injected followed by glycemia measurement over 120 min using a glucometer (Contour®). NB: At the 24 hours time-point, the test was stopped after 30-45 minutes as the glycemia of the mice was severely dropping and started showing moderate to severe hypoglycemia. The results are shown in FIGS. 61A to 61C.

### Example 11. Tirzepatide-Transfer Reagent Preparation and Analysis

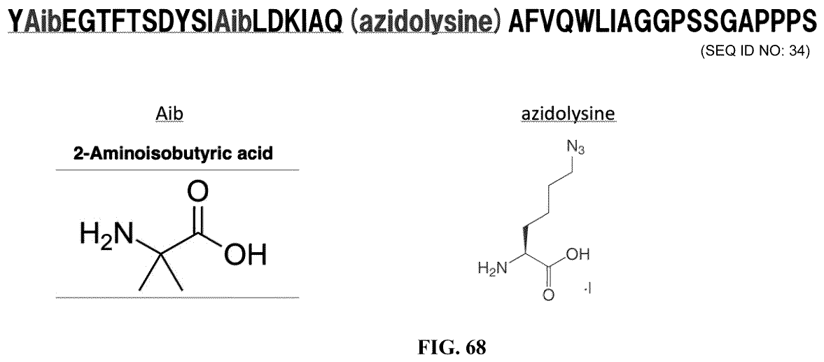

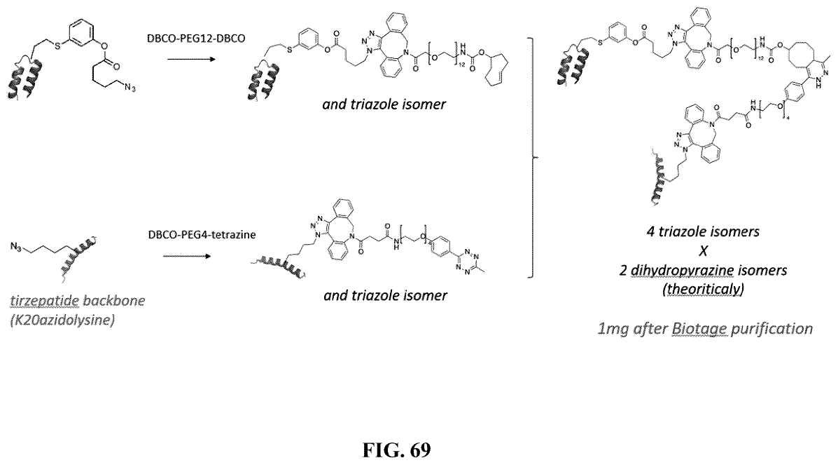

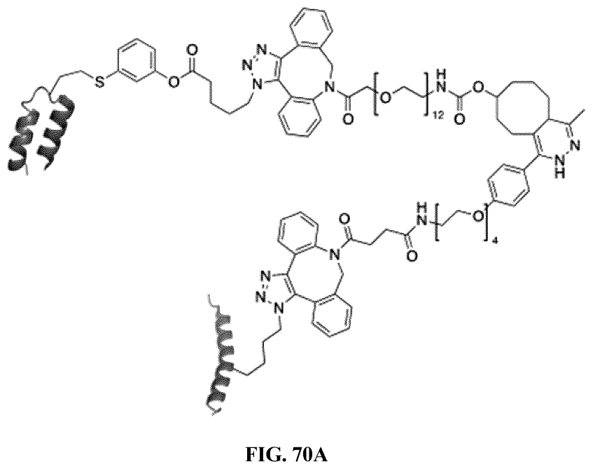

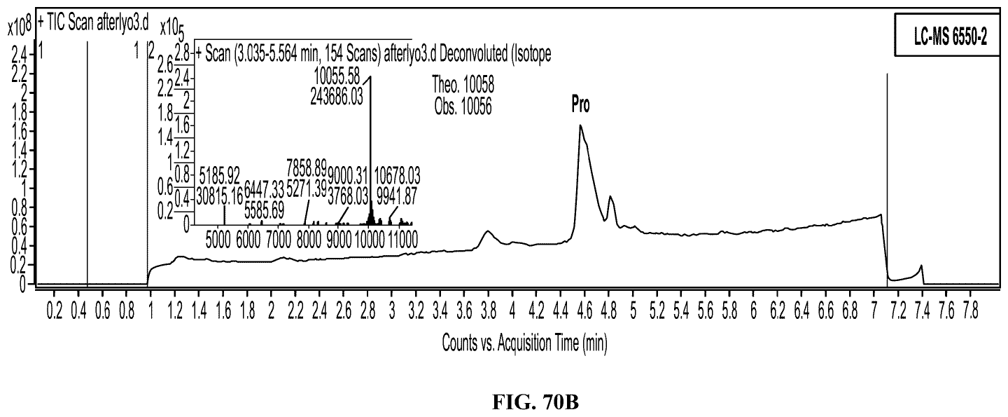

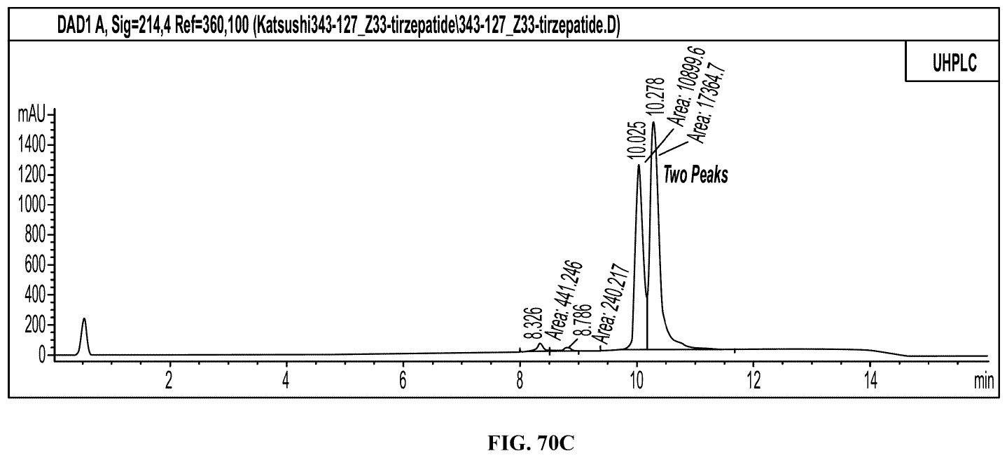

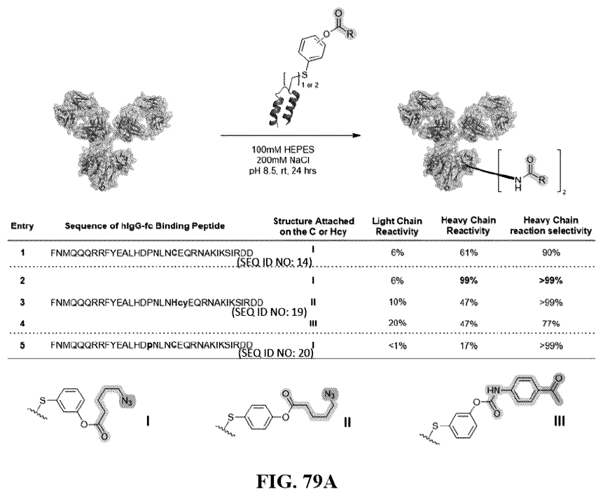

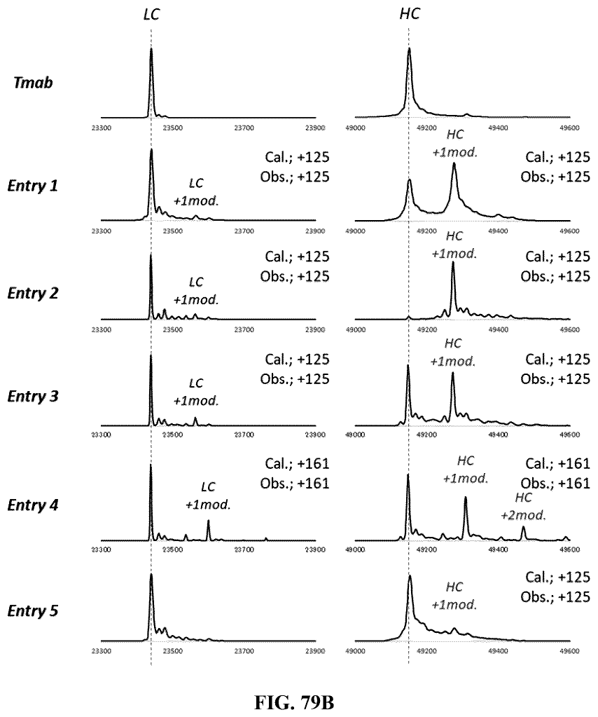

A tirzepatide-transfer reagent was synthesized as described previously (FIG. 68 and FIG. 79). The purified tirzepatide-transfer reagent (FIG. 70A) was characterized using LC-MS (FIG. 70B) and UHPLC (FIG. 70C), as previously described. LC-MS analysis confirmed the presence of the tirzepatide-transfer reagent (theoretical: 10058; observed: 10056). The initial purification conditions yielded a ˜50% purity.

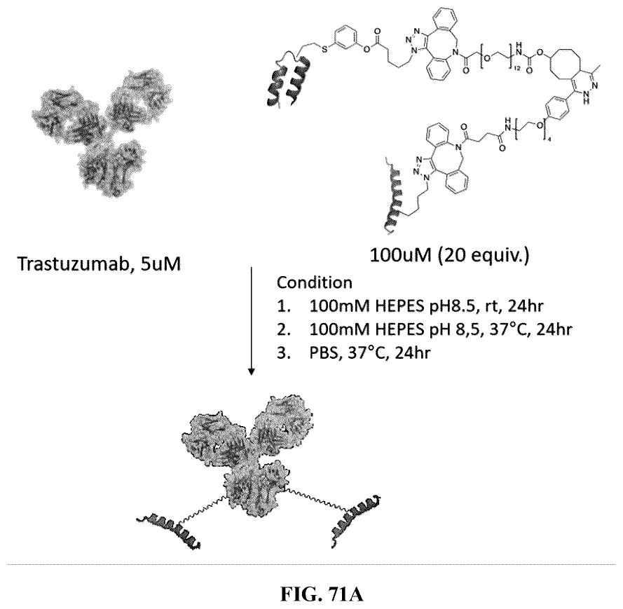

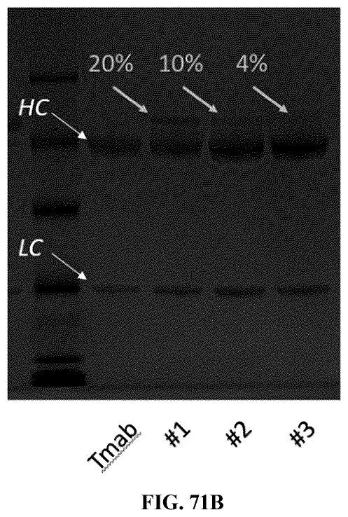

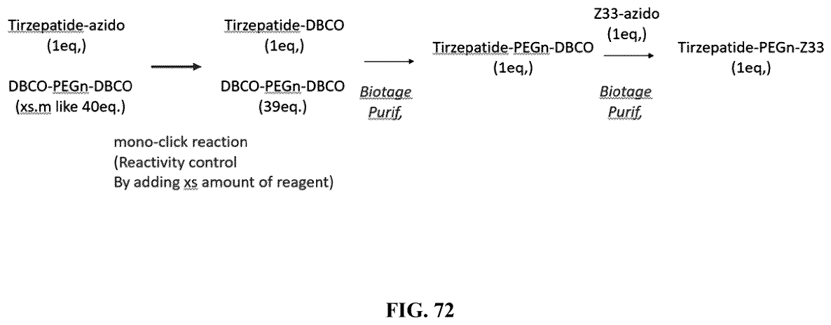

The purified tirzepatide-transfer reagent was then evaluated for its reactivity against human IgG trastuzumab. Briefly, 5 μM of trastuzumab was reacted with 100 μM (20 equivalents) of the purified tirzepatide-transfer reagent under one of three conditions: 1) 100 mM HEPES pH 8.5 at room temperature for 24 hours; 2) 100 mM HEPES pH 8.5 at 37° C. for 24 hours; and, 3) PBS at 37° C. for 24 hours (FIG. 71A). The resulting reaction was evaluated using gel electrophoresis, which confirmed that all three conditions yielded a reacted product (FIG. 71B). However, the conversion ratio was low at around 4-20%. A new synthetic plan to obtain pure tirzepatide-transfer reagent is shown in FIG. 72).

### Example 12. mAB Binding Peptide Z33 and its Cysteine Variants

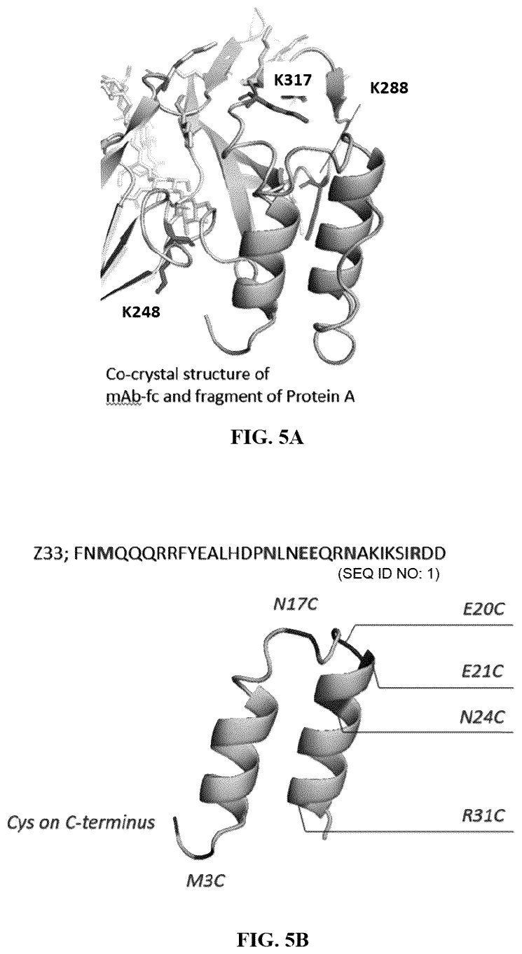

Synthesis of IgG binding peptide Z33 analogs were performed as described in Example 3. A co-crystal structure of mAB-FC and Z33 was used to determine lysine residues in the active site (FIG. 5A). Based on this information, seven Z33-cysteine variants were designed and synthesized (FIG. 5B). The seven mutation sites chosen were: Cys on C-terminus, M3C, N17C, E20C, E21C, N24C, and R31C.

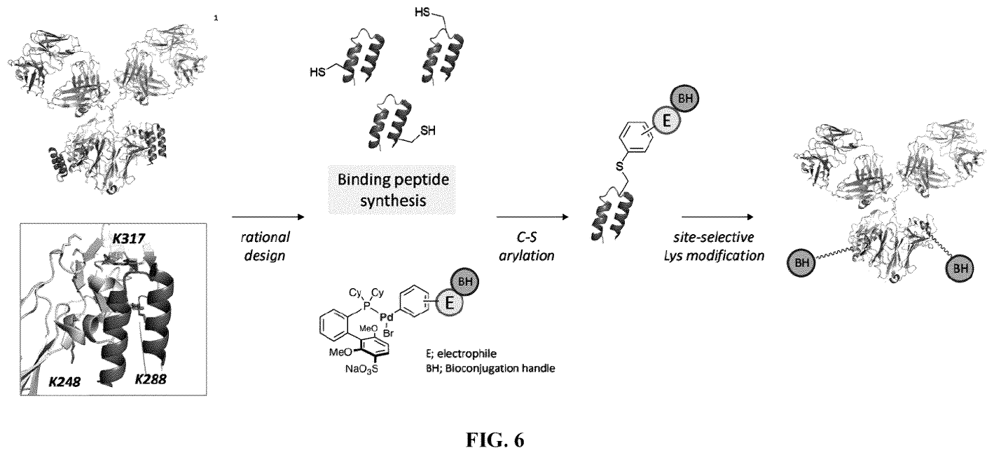

Each of the Z33 analogs containing cysteine were designed with the suitable distance for each lysine in mind. At the same time, palladium oxidative addition complex with finely designed electrophilic positions were prepared. The electrophile-attached IgG binding peptides were prepared by C-S arylation for selective IgG modification, as described previously (FIG. 6).

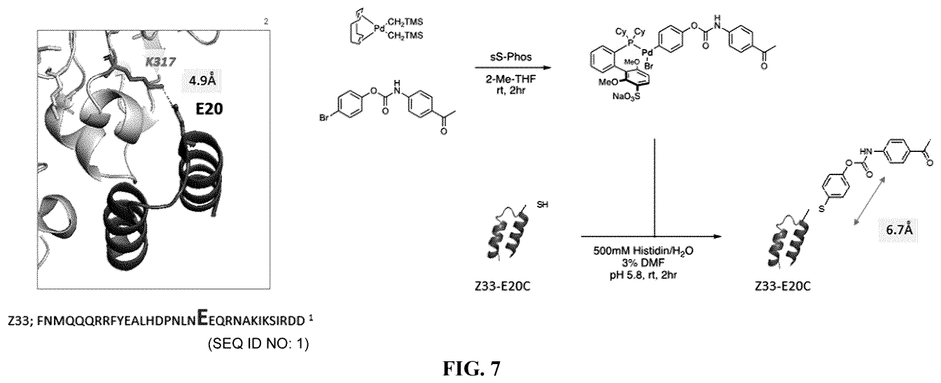

The first target was K317 modification using the Z33 peptide as the IgG binding peptide (FIG. 7). The Z33 is part of the protein A and consists of 33 amino acids. The glutamic acid at position 20 was determined by co-crystal structure to be located close to this K317 residue. To synthesize an aryl-carbamate attached Z33, Pd OAC was prepared from carbamate-containing aryl bromide, followed by C-S arylation to Z33-E20C.

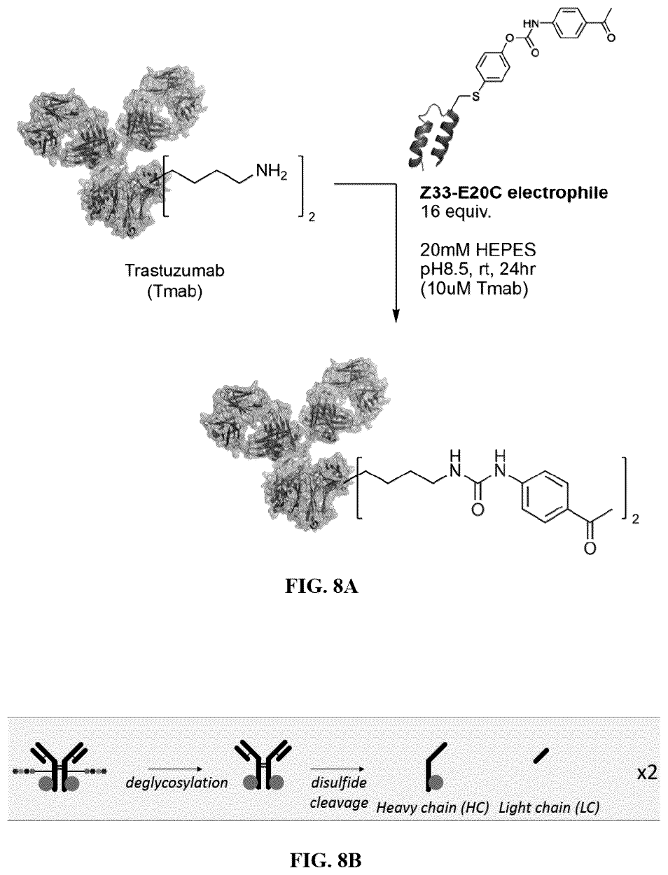

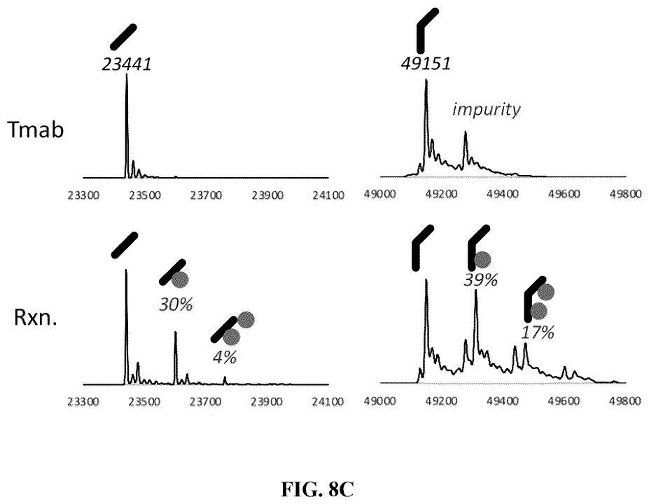

The reaction was set up using trastuzumab as the IgG under basic pH conditions (FIG. 8A) and analyzed using mass spectrometry. The glycan on the IgG makes it difficult to analyze the data because of its heterogeneity. Additionally, IgG has a low ionization ability due to its large molecular weight. Thus, to analyze the trastuzumab modification, a de-glycoslyation and disulfide cleavage reaction was performed (FIG. 8B). If the modification occurs on the heavy chain, as expected, only one modified heavy chain is detected. FIG. 8C shows the Tmab light chain (top left) and Tmab heavy chain (top right), which contains the desired modified heavy chain (49151) as well as undesired peaks.

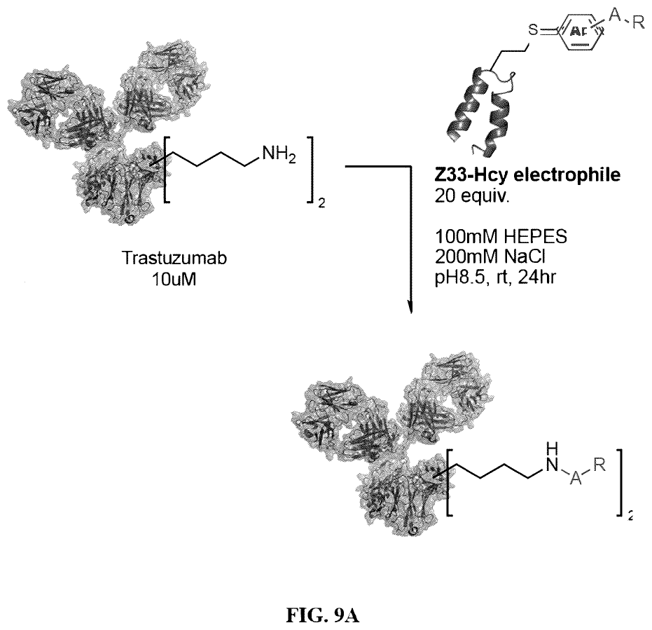

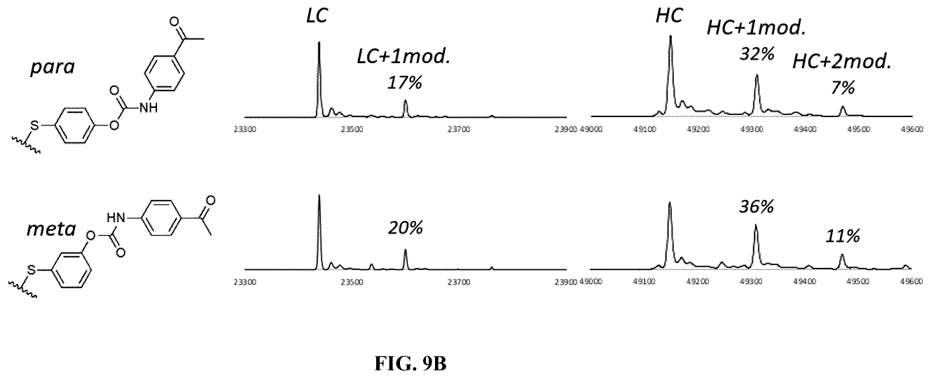

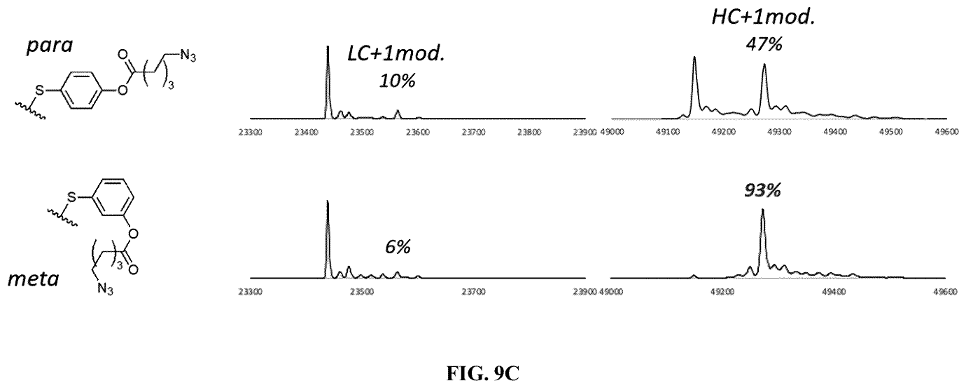

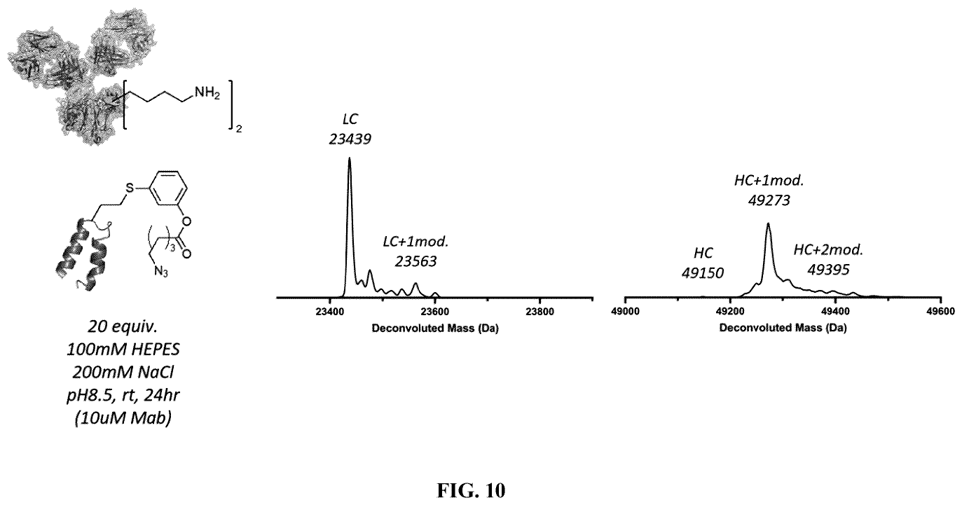

To evaluate whether quantitative modification could be achieved by changing the electrophile, a homocysteine was introduced in place of cysteine to optimize the distance between IgG and Z33 (FIG. 9A). A small improvement in selectivity was observed (FIG. 9B). Meta-substituted electrophiles were also synthesized, but there was not a significant improvement in the selectivity. To reduce steric hindrance around the carbonyl group and to reduce the nonspecific reactivity, a phenyl ester was used as the electrophile. Finally, a met-substituted phenyl ester was used and resulted in 93% conversion rate and high selectivity (FIG. 9C). Most of the heavy chain was found to be modified by the 5-aazido-pentanoic acid-3-phenylester reagent. A detailed LC-MS analysis of the light and heavy chain is shown in FIG. 10, which shows that in addition to the high conversion rate, only a small percent of undesired light chain modification and over-modification was detected.

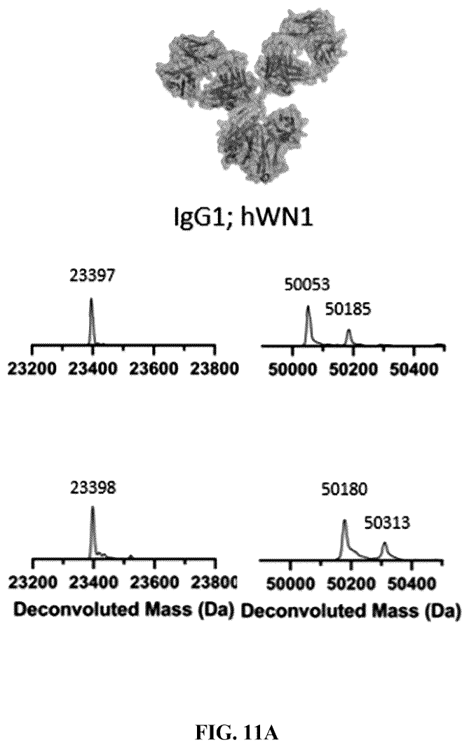

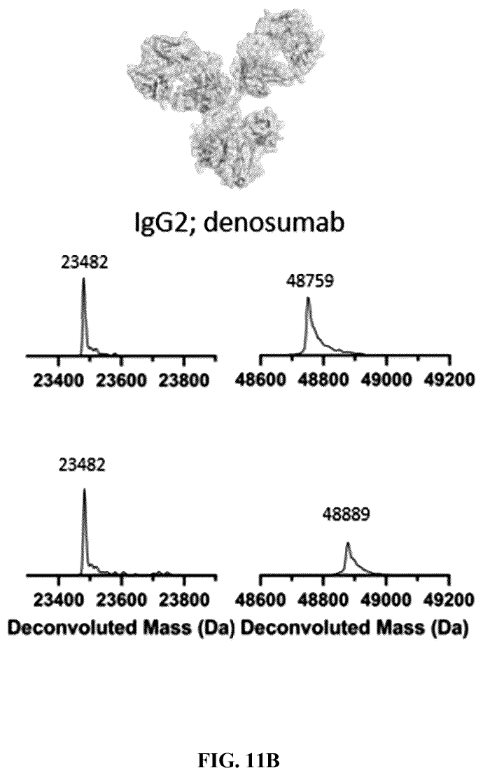

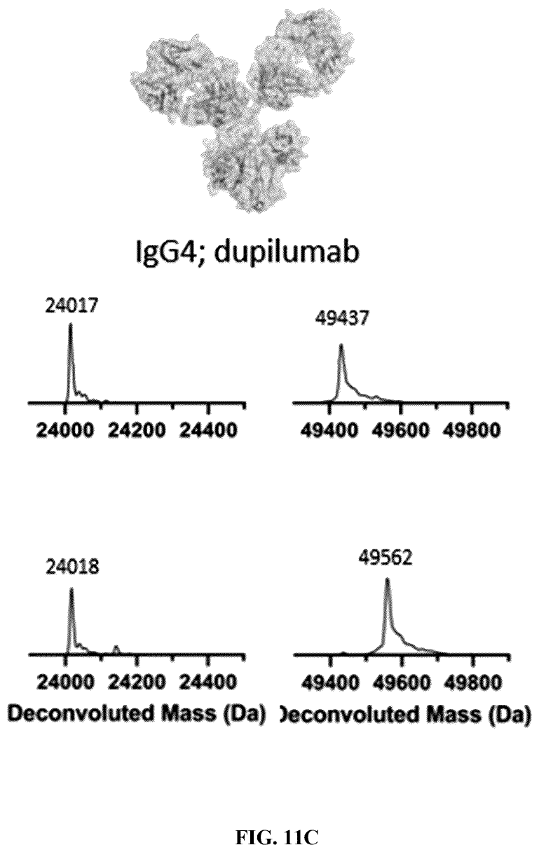

To demonstrate the generality of this reaction, other IgGs were used to determine reactivity with Z33. WN1 was used as another IgG1 (FIG. 11A), denosumab as IgG2 (FIG. 11B), and dupilumab as IgG4 (FIG. 11C). The LC-MS results showed that in all cases, the majority of the heavy chain was modified.

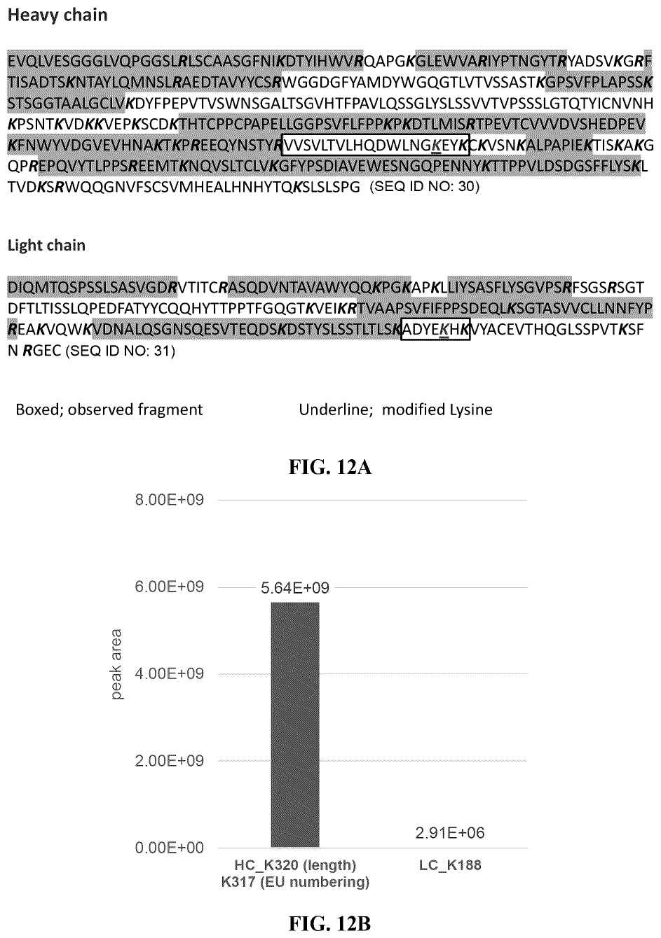

Samples were then evaluated using Orbitrap LC-MS/MS to confirm the modification site and determine which lysine reacted with the reagent. A number of fragments were detected (FIG. 12A), and the peptide mapping result indicated that the modification mainly proceeded on the heavy chain at K317 (FIG. 12B).

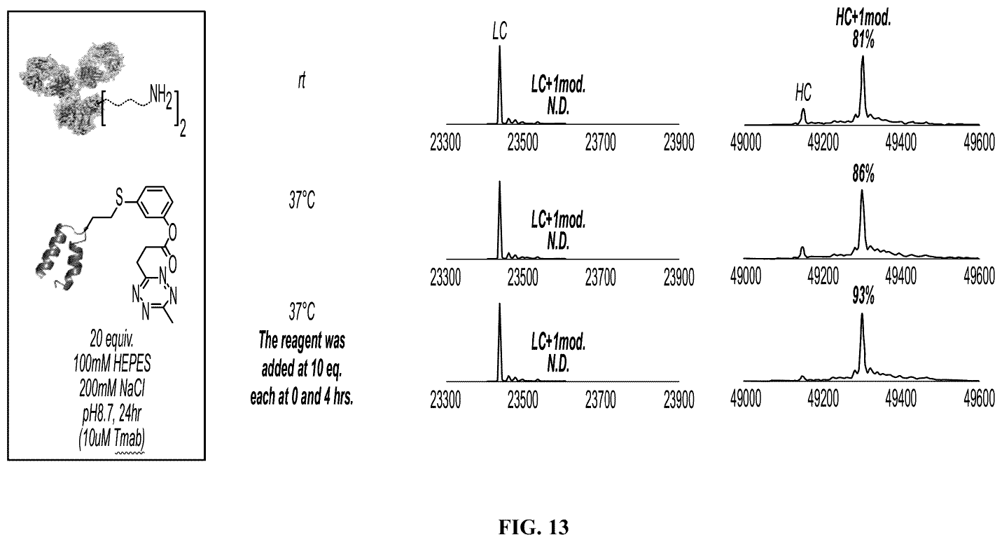

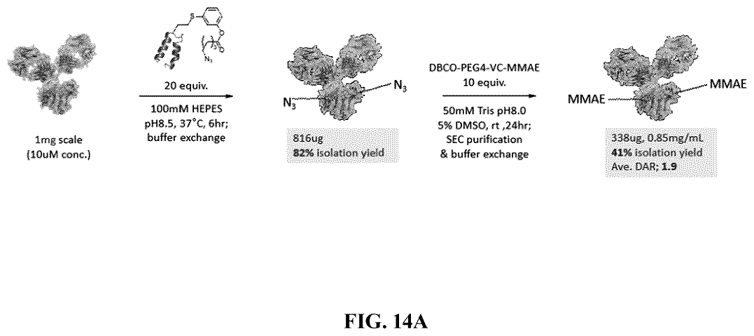

Next, other bioconjugation handles were tested to further demonstrate the generality of this reaction. Tetrazine was reacted as described above. LC-MS analysis of the reaction at three different conditions demonstrated that the reagent with tetrazine had a similar reactivity to that with azido under similar conditions (FIG. 13). By running the reaction at 37° C. and adding the reagent in two portions (at 0 and 4 hours), a 93% conversion rate was achieved.

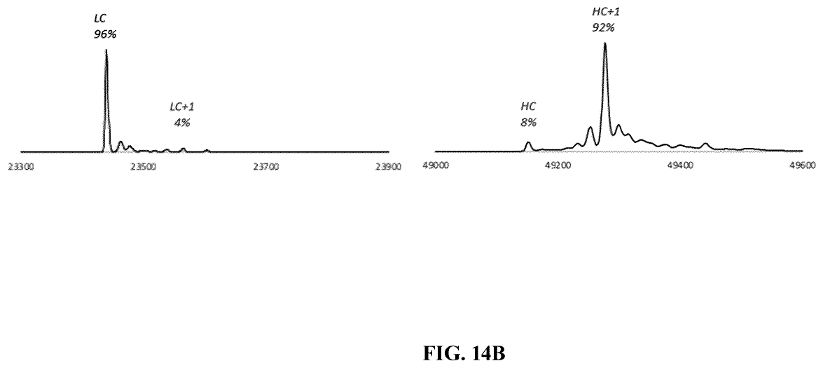

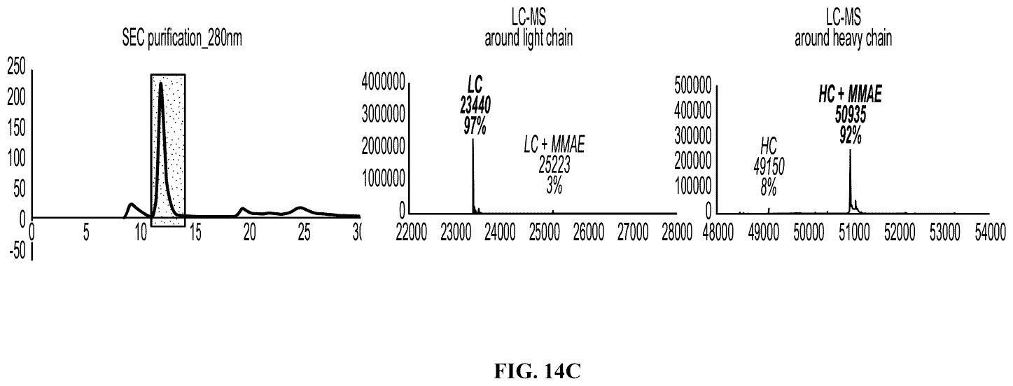

One of the purposes of this technology is to create selective ADCs. The reaction was optimized and demonstrated that selective modification worked well even at the 1 mg scale (FIG. 14A). A click-reaction with DBCO-attached anticancer drug MMAE proceeded quantitatively. After purification with SEC, the desired ADC was obtained in good isolation yield and its DAR (at 1.9) was highly controllable (FIGS. 14B to 14C).

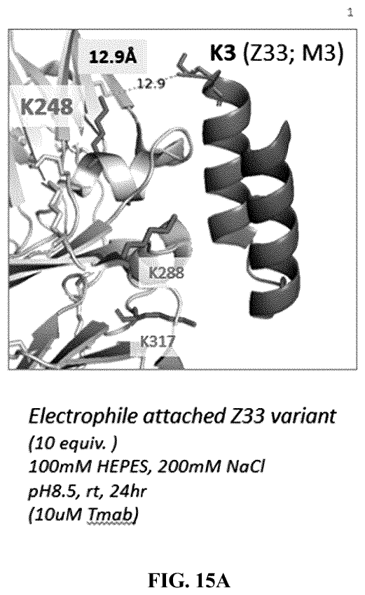

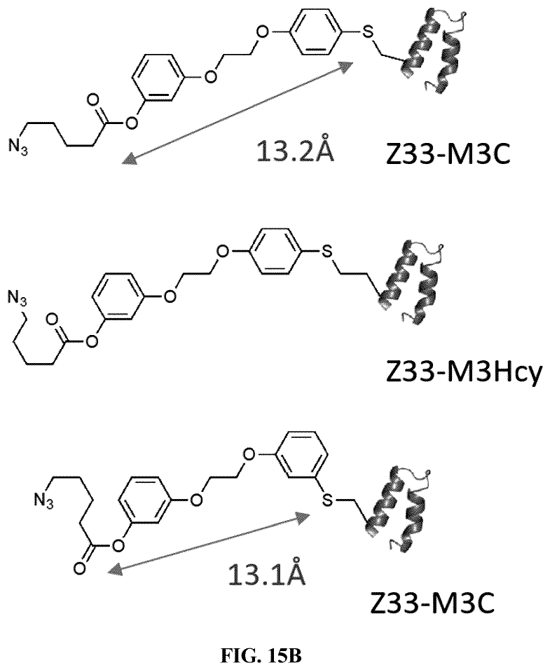

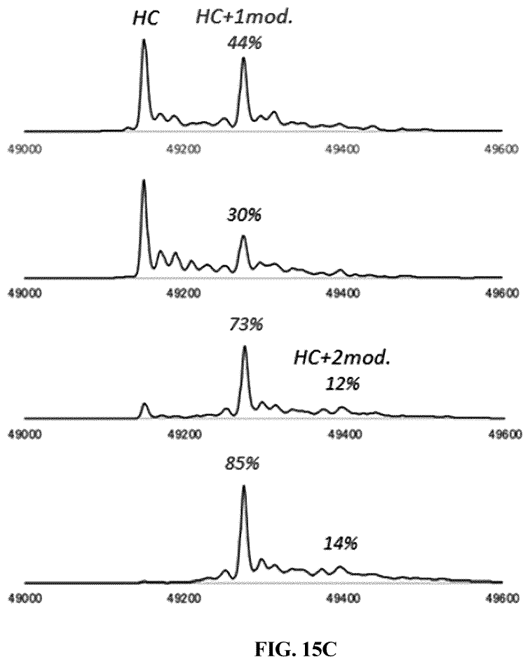

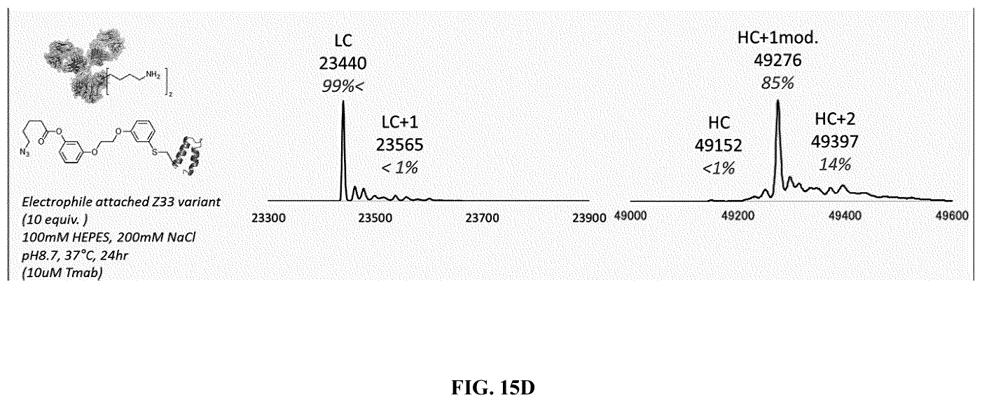

A second lysine modification was then performed to evaluate whether the electrophile with a longer linker could react with lysine 248. The second lysine targeted was K248, which is located close to the M3 in Z33 (FIG. 15A). The distance between these two residues is 12.9 angstroms, so a long linker was required. This electrophilic Z33 reagent was designed (FIG. 15B) and found to have a moderate conversion rate (FIG. 15C). When the homocysteine variant of Z33 was used, the conversion rate decreased. The conversion rate increased to 73% when using a meta-substituted electrophile in the cysteine variant. The conversion rate was further increased to 85% by performing the reaction at 37 degrees. A small amount of over-modification was observed, but the primary reaction result was mono modification of the heavy chain (FIG. 15D), indicating that the flexible linker design worked well and produced great reactivity.

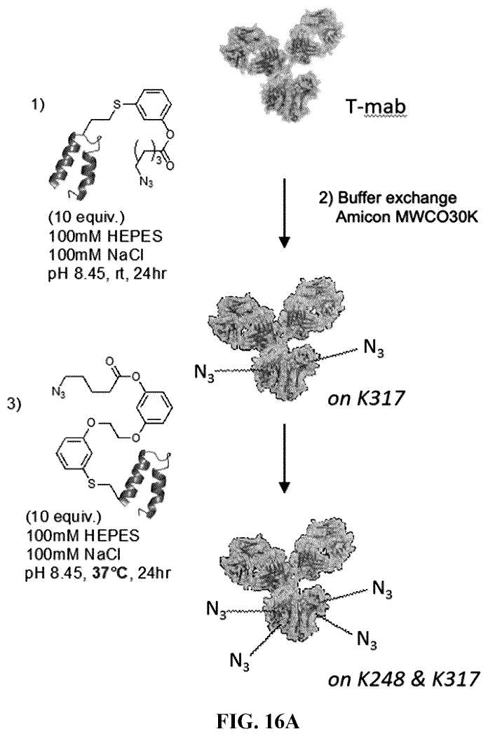

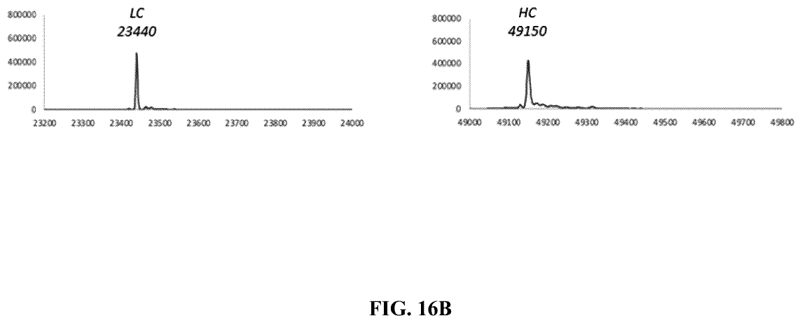

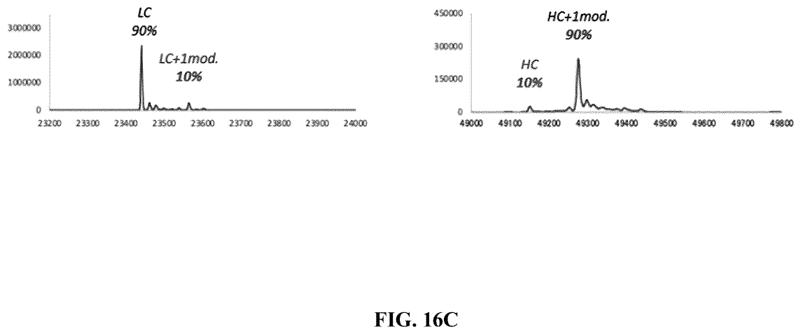

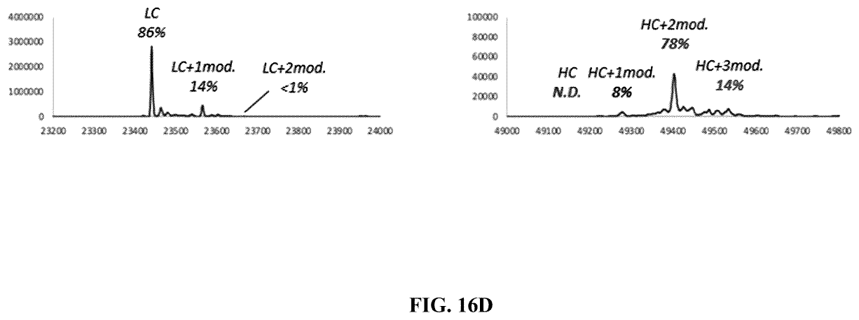

The next step was to achieve double modification of the K317 and K248 on the same IgG. When the previous reagents were reacted sequentially with IgG, the double modified IgG was obtained as the main product and each modification reactivity was similar to the reactivity in the single modification (FIGS. 16A to 16D).

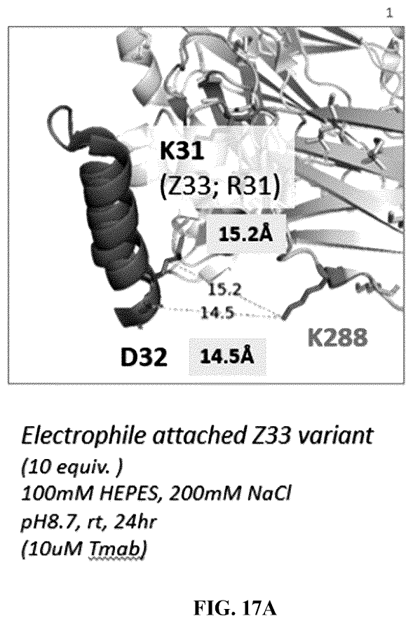

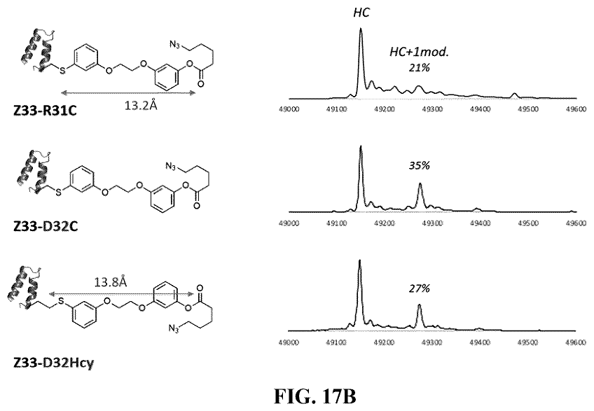

Finally, a third lysine modification K288 was evaluated using a long linker. The R31 and D32 were shown to be located close to the K288 (FIG. 17A), so the electrophilic Z33 was prepared as previously described. Though a clear peak was not observed using the R31-Z33 variant, 35% of the modified heavy chain was observed in the D32 variant (FIG. 17B). No significant improvement was observed using the homocysteine Z33 variant.

### Example 13. In Vivo IgG Functionalization

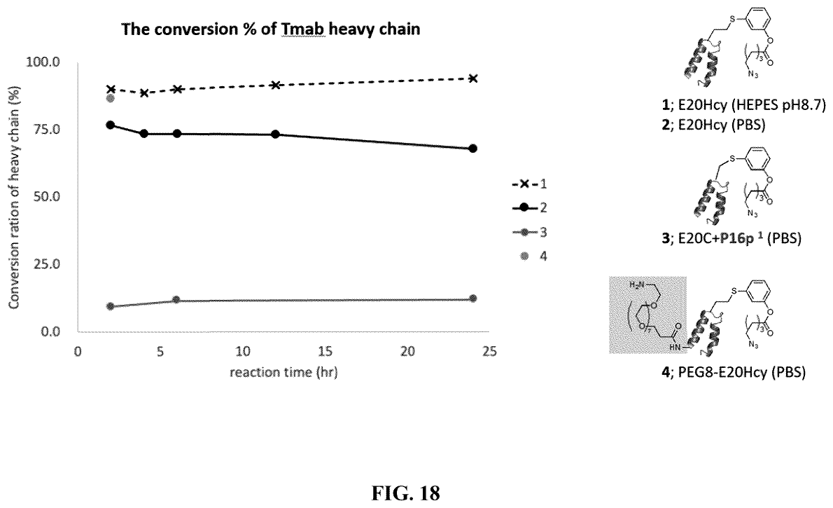

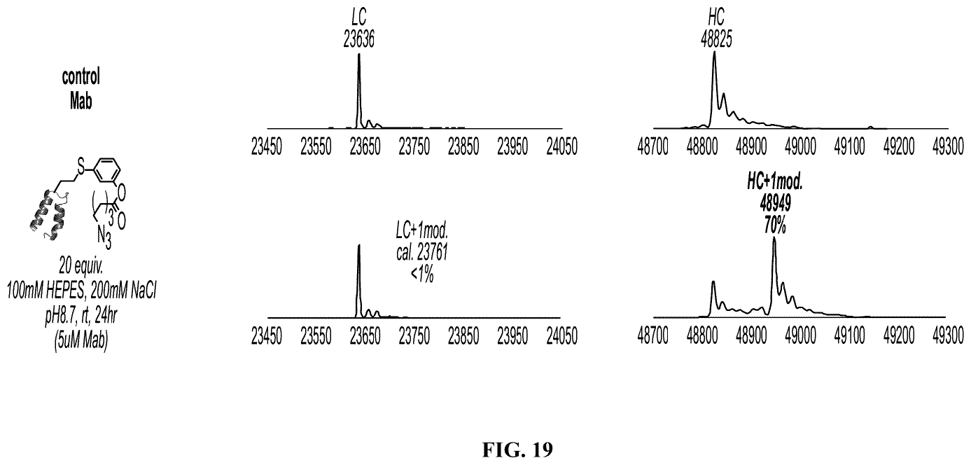

To modify IgG in vivo, the reagents need to react in neutral pH conditions. The reactivity of the reagents was evaluated using PBS and found that more than about 70% of Tmab was modified (FIG. 18). Additionally, the reagent containing D-proline instead of proline showed less conversion (at around 10%), indicating that it could be used as a negative control in further experiments. Additionally, species differences need to be considered for future mouse experiments, so the reactivity was evaluated in mouse IgG. LC-MS analysis confirmed that the heavy chain mouse IgG was selectively modified (FIG. 19).

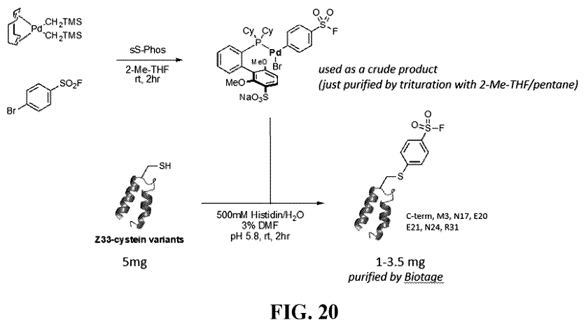

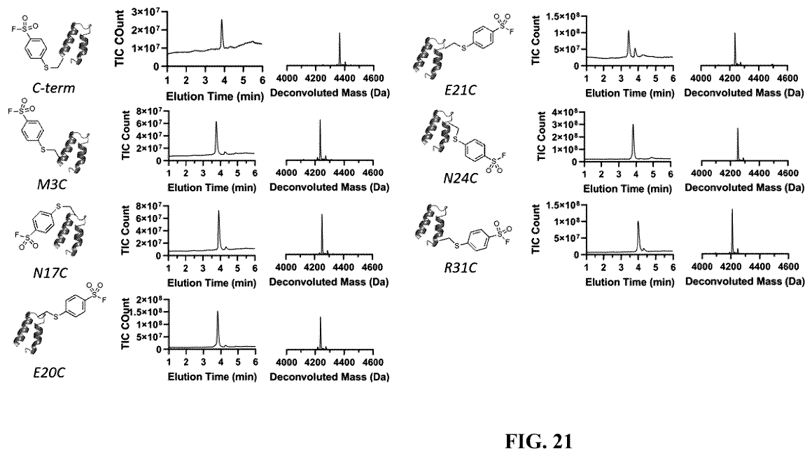

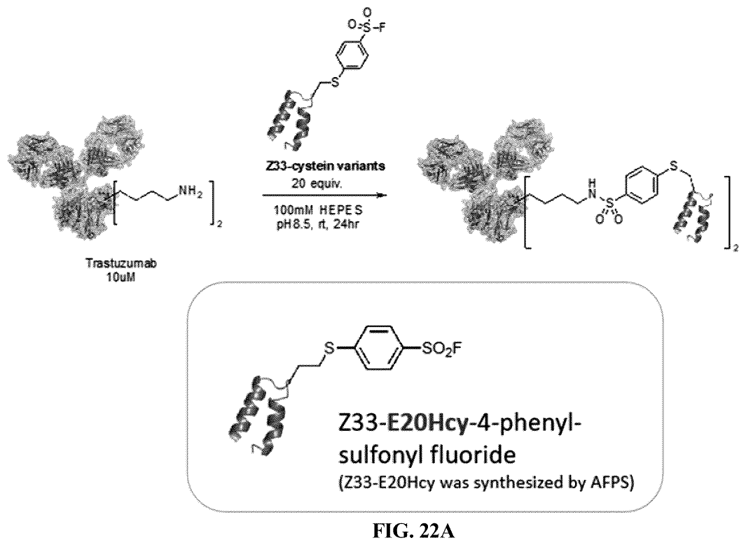

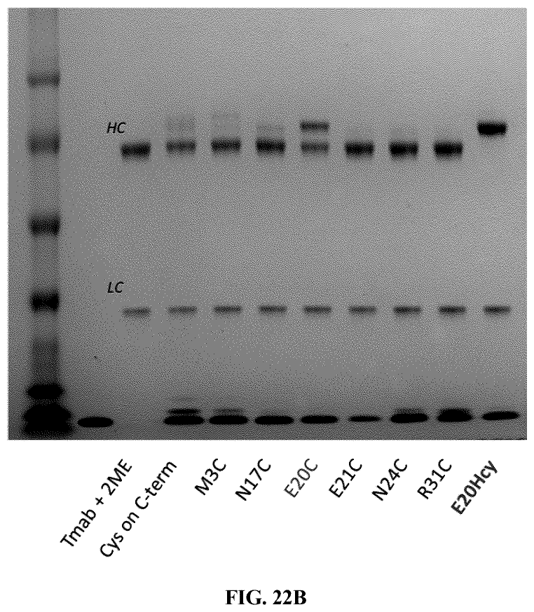

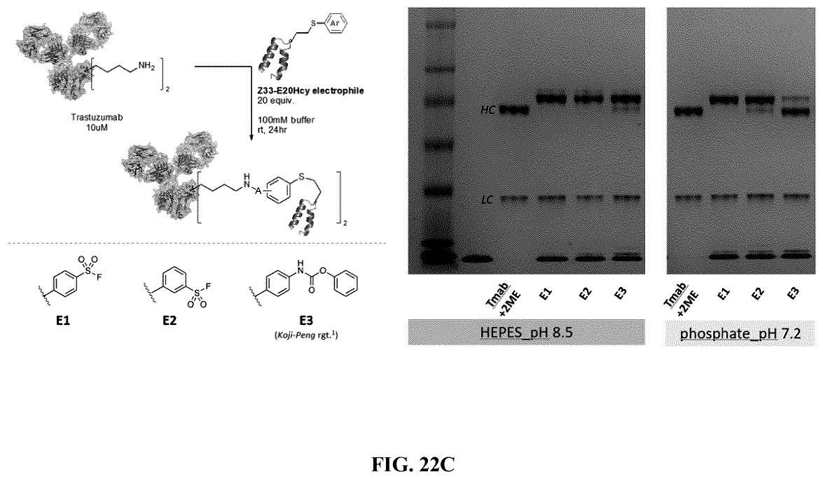

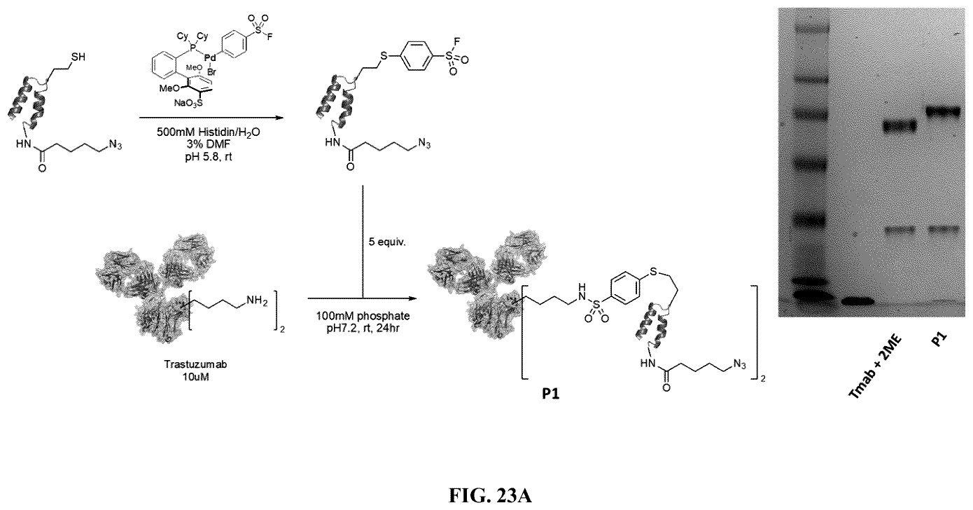

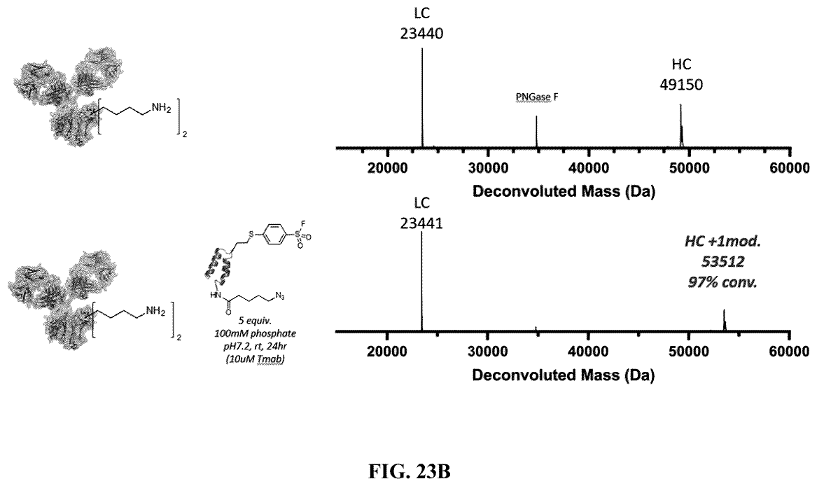

The nucleophilic Z33 variants were each prepared in a single step (FIG. 20) and evaluated using LC-MS (FIG. 21). Each variant was screened to determine which residue was important to attach the electrophile. Quantitative modification was observed using the Z33-E20Hcy variant (FIGS. 22A to 22B). Further, for antibody modification, meta and para SO2F were shown yield the best results (FIG. 22C). Next, azide group incorporation of Z33-E20Hcy was evaluated and found that quantitative modification was observed (FIG. 23A). LC-MS analysis revealed that azide-attached Z33-E20Hcy yielded a 97% conversion rate (FIG. 23B).

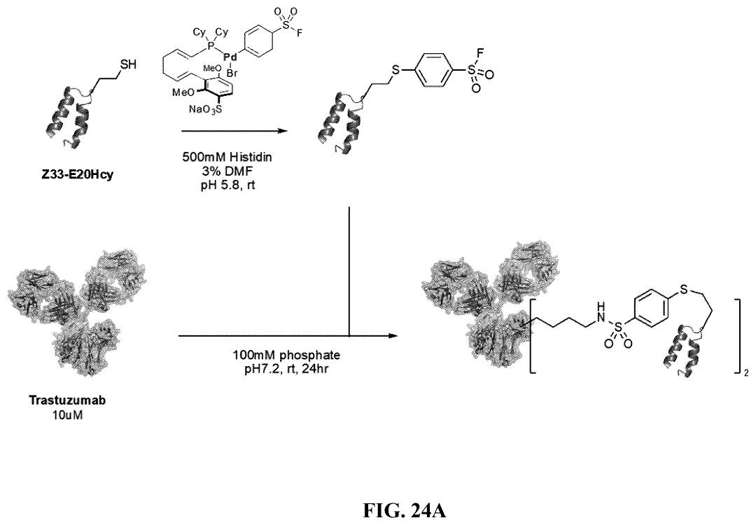

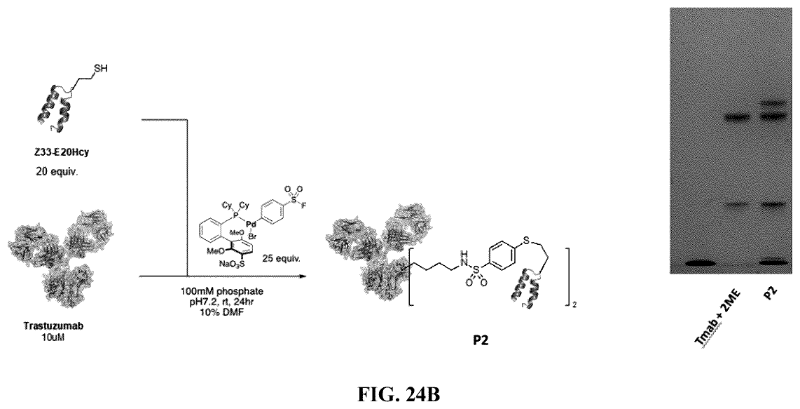

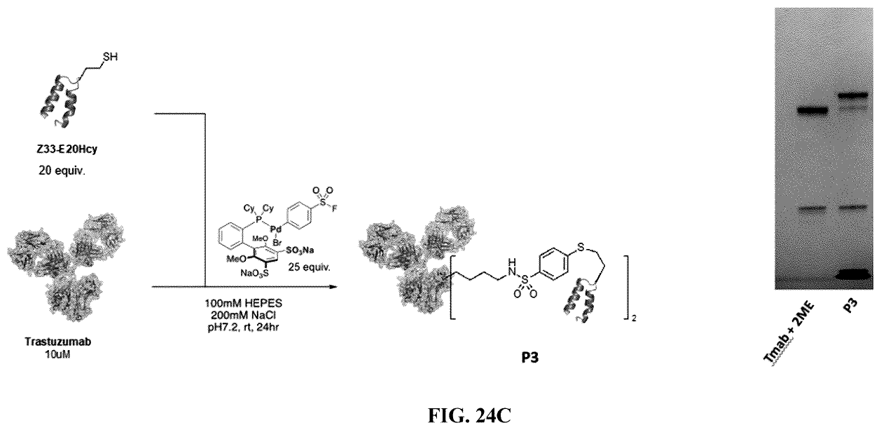

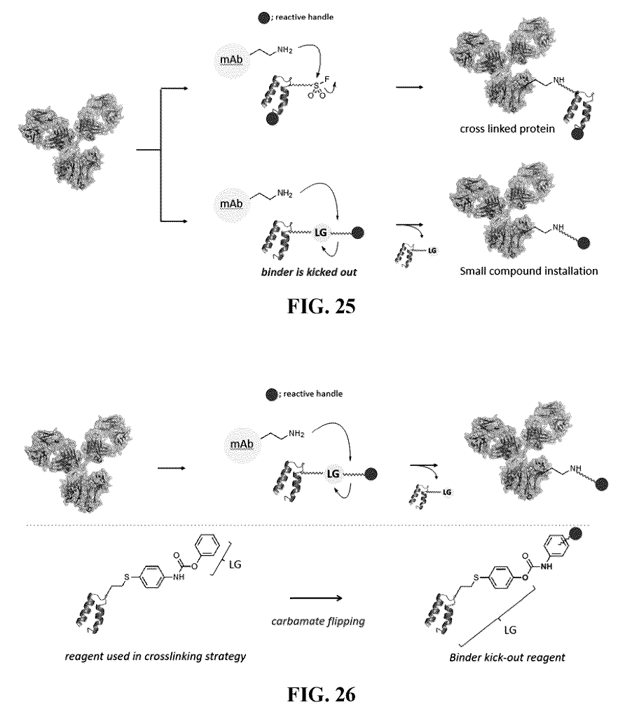

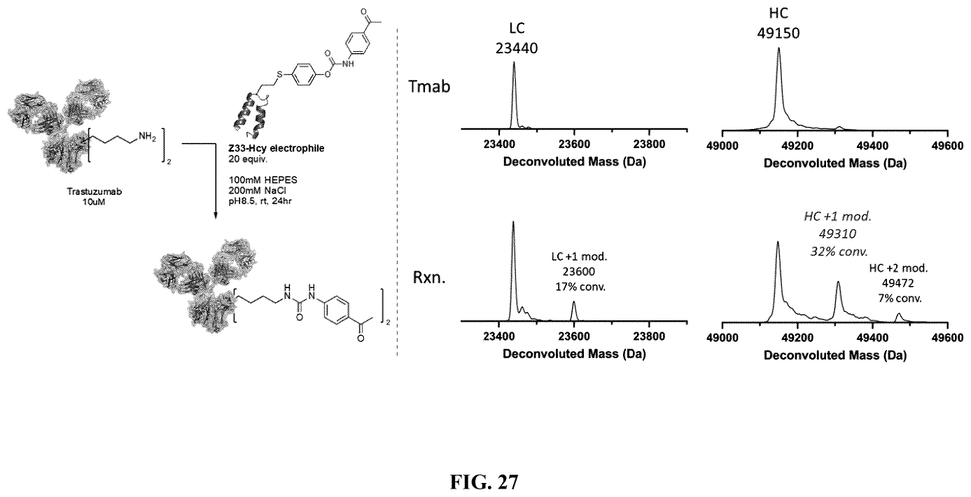

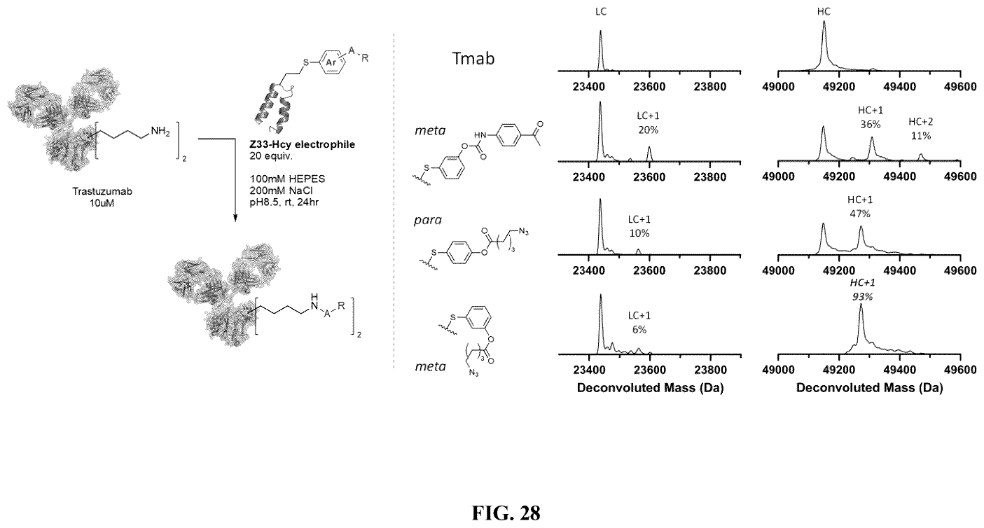

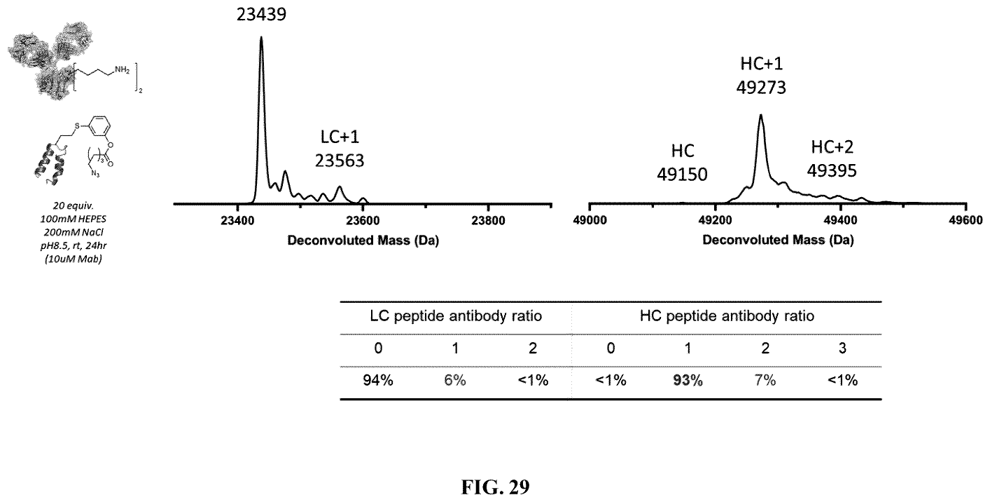

Next, a one-pot procedure was tested to determine whether it was necessary to isolate and purify the electrophilic Z33 (FIG. 24A). FIGS. 24B to 24C show that most of the heavy chain was modified by just mixing the reagents. Another modification approach was proposed using a mAB binder kick-out strategy, which could be useful for small compound installation (FIG. 25). The reagent was designed by flipping the carbamate of the protein crosslinking agent (FIG. 26). LC-MS analysis revealed that the flipped carbamate reagent worked, but its reactivity and selectivity were not sufficient (FIG. 27). To further evaluate this, the electrophile was changed to determine if quantitative modification could be achieved. FIG. 28 and FIG. 29 show that most of the heavy chain was modified by 5-azido-pentanoic acid-3-phenylester reagent.

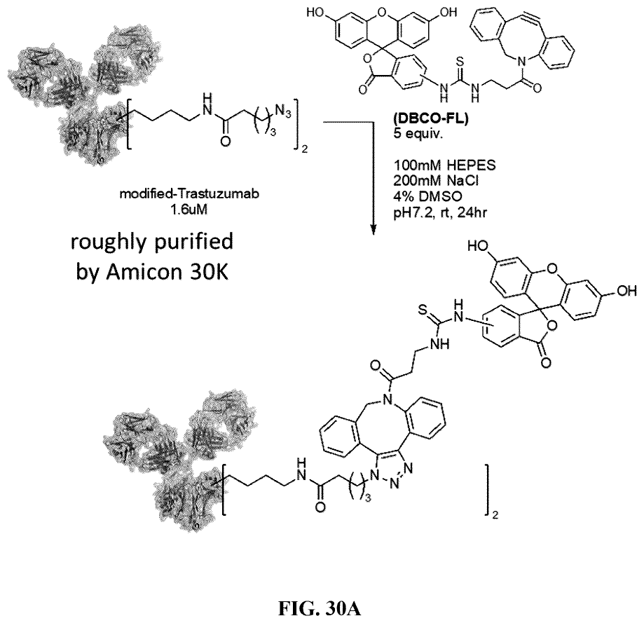

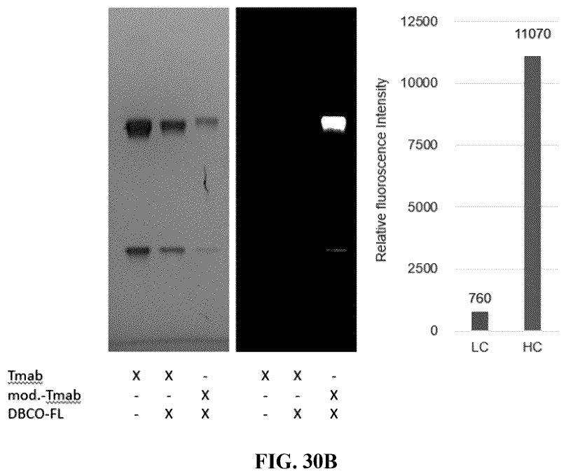

Small compound conjugation via the azide group was evaluated for its reactivity with DBCO (FIG. 30A). Gel electrophoresis analysis indicated that the click reaction occurred and verified that azide would work as a future reactive handle for drug conjugation (FIG. 30B).

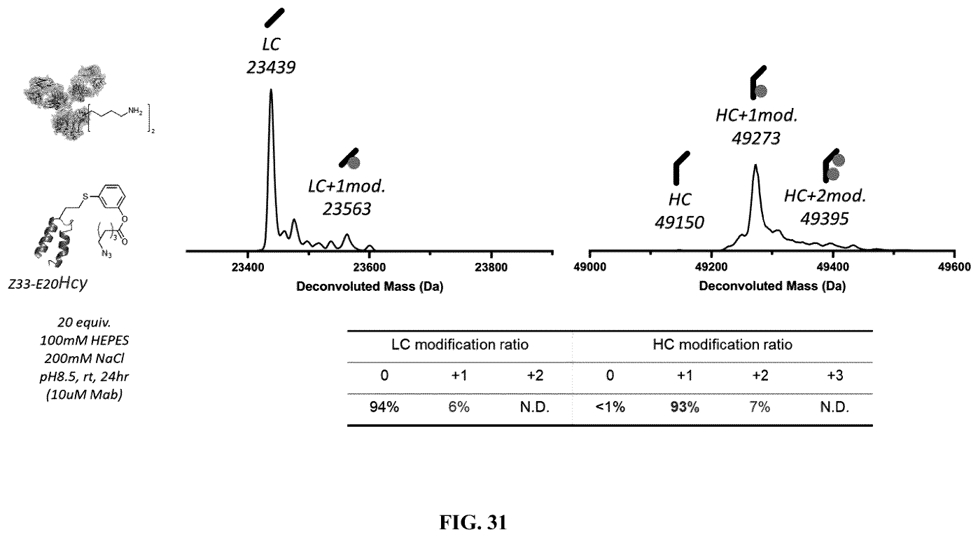

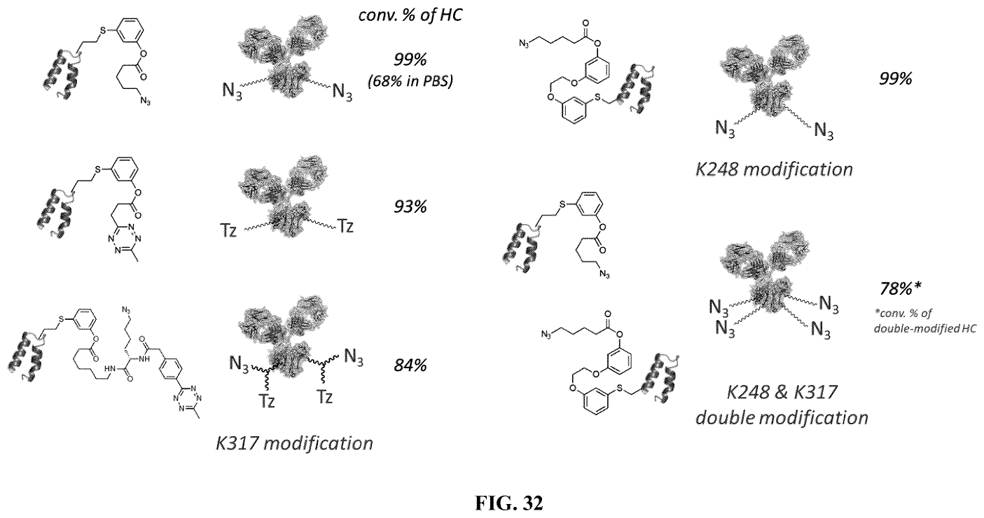

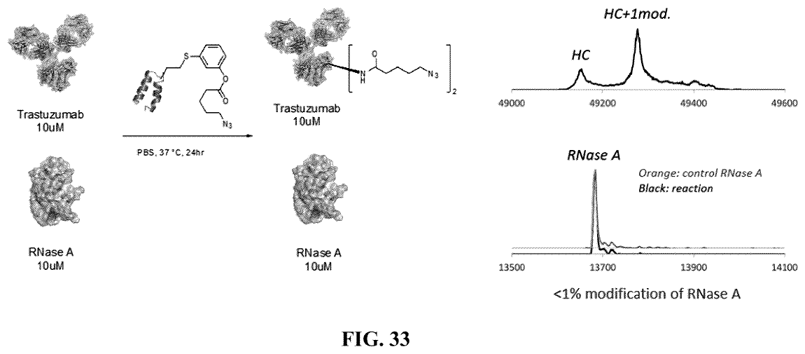

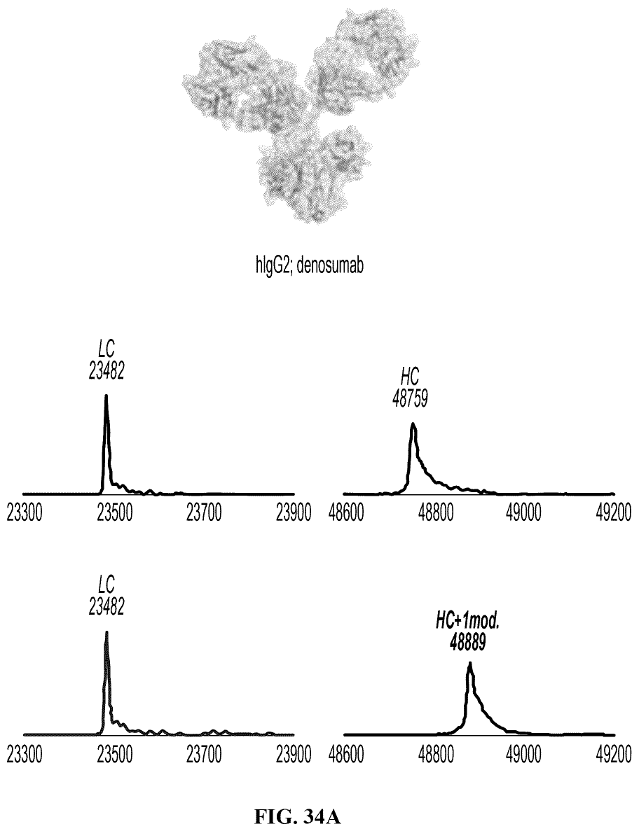

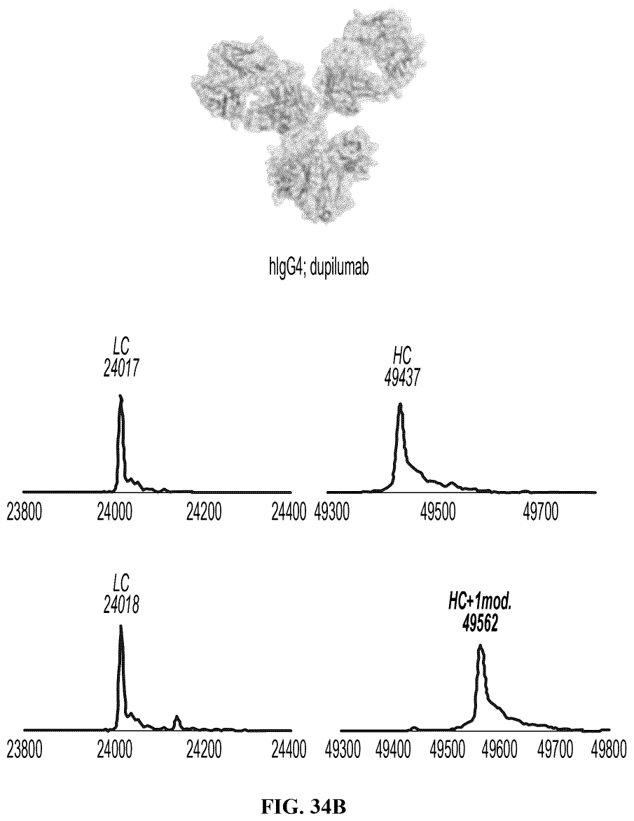

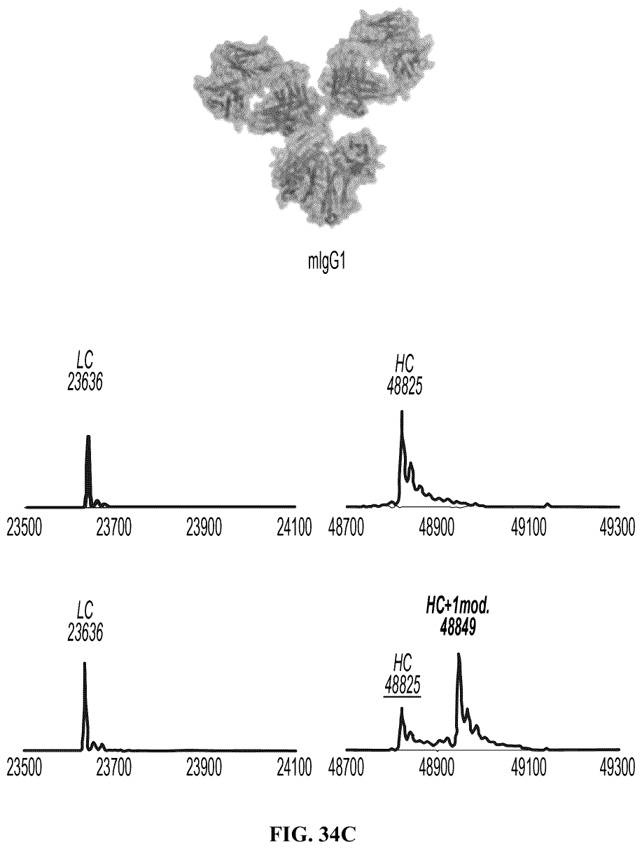

Thus, successful IgG modification was demonstrated in vitro and showed that most of the heavy chain could be modified by 5-azido-penatnoic acid-3-phenylester reagent (FIG. 31). The results indicate a number of successful examples of site-selective IgG modification and double modification (FIG. 32). Specific reactivity towards IgG was also demonstrated and showed that IgG1 trastuzumab was selectively modified by the reagent (FIG. 33). Further, human IgG1, human IgG2, human IgG4, and mouelgG1 were also successfully modified by Z33 electrophiles (FIGS. 34A to 34C).

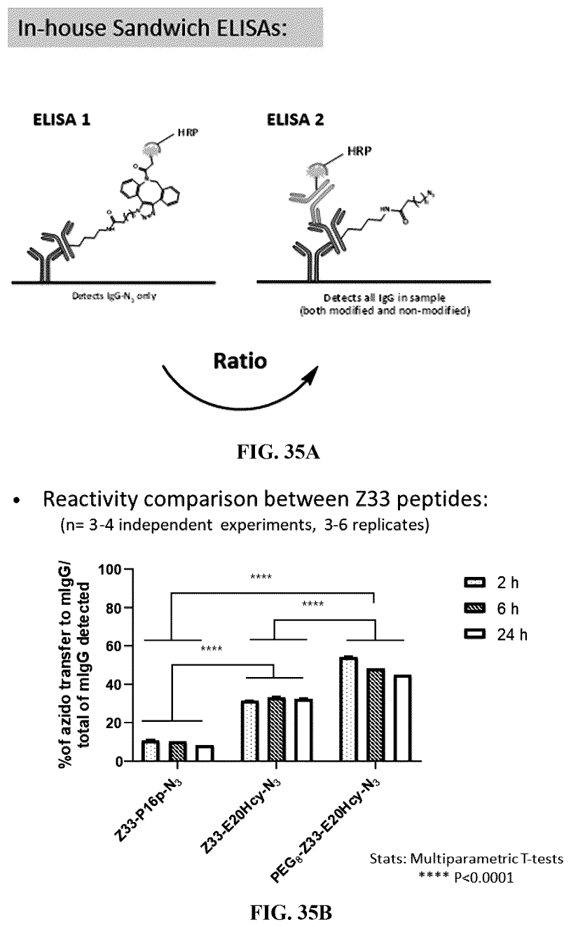

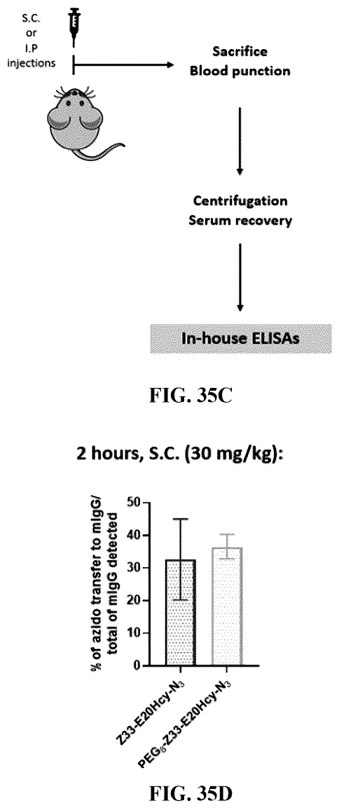

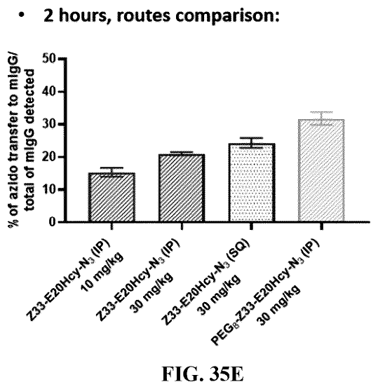

An in-house sandwich ELISA was used to evaluate whether Z33 peptides react to mouse IgG when incubated in mouse serum (FIG. 35A). The results demonstrated that azido transfer was effective on mouse IgG in vitro, and was significantly more efficient with pegylated Z33 (FIG. 35B). When comparing the routes of administration (FIG. 35C), slight differences were found between subcutaneous and intraperitoneal routes (FIGS. 35D to 35E) however azido transfer was effective in both in vivo.

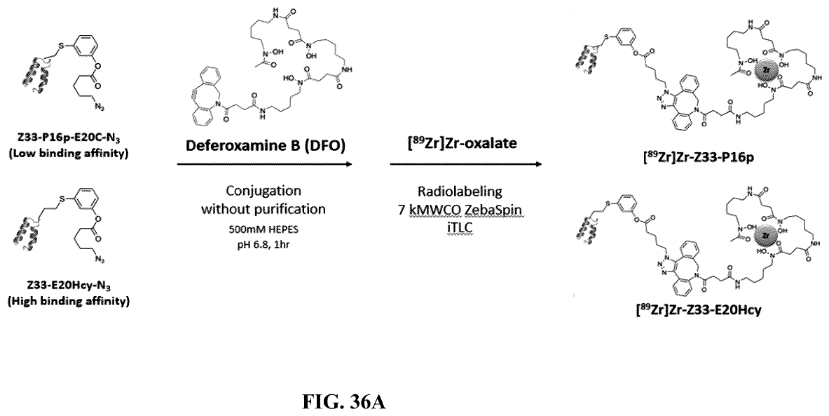

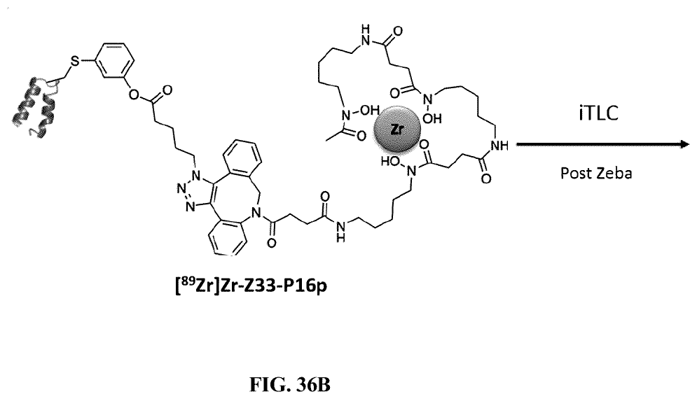

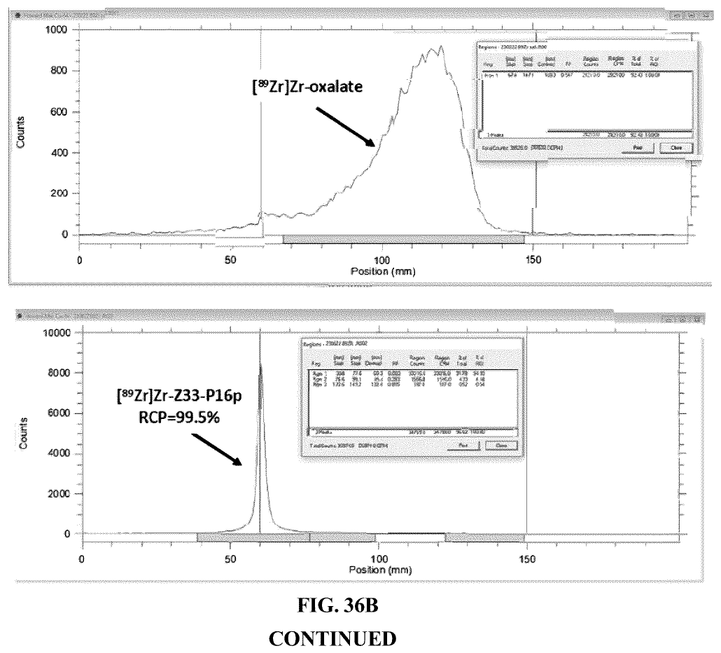

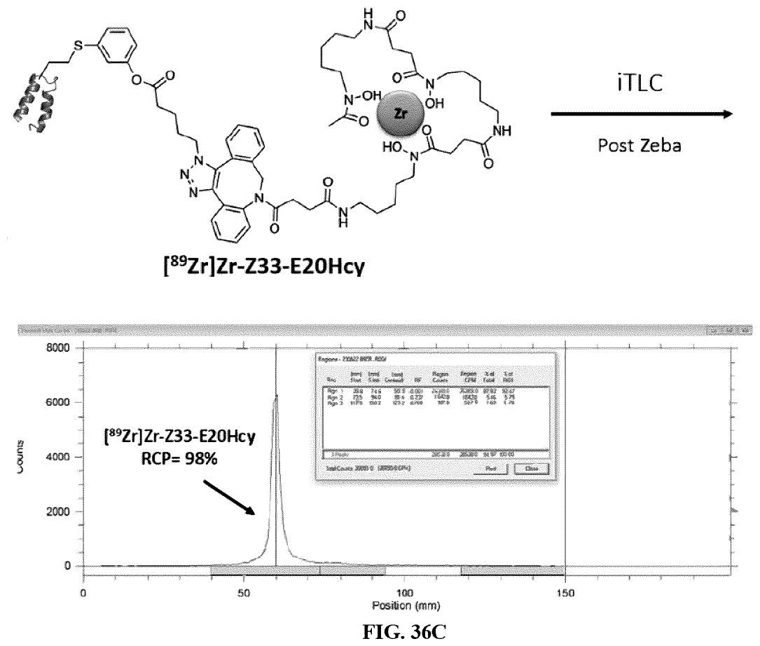

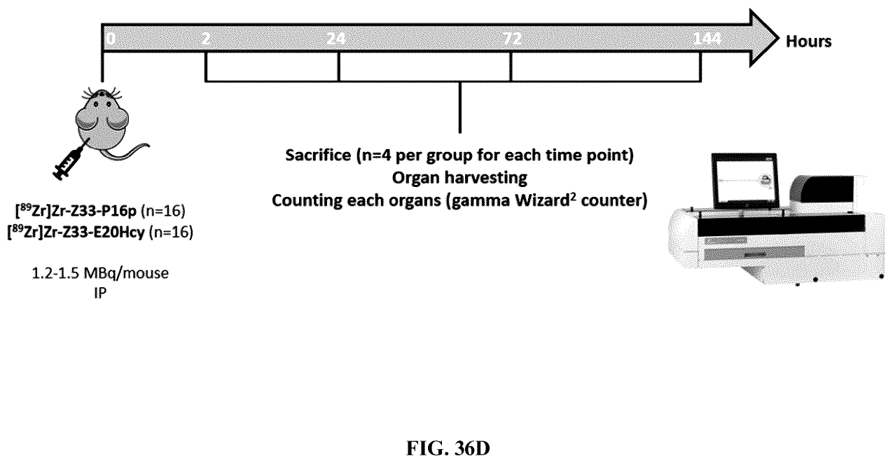

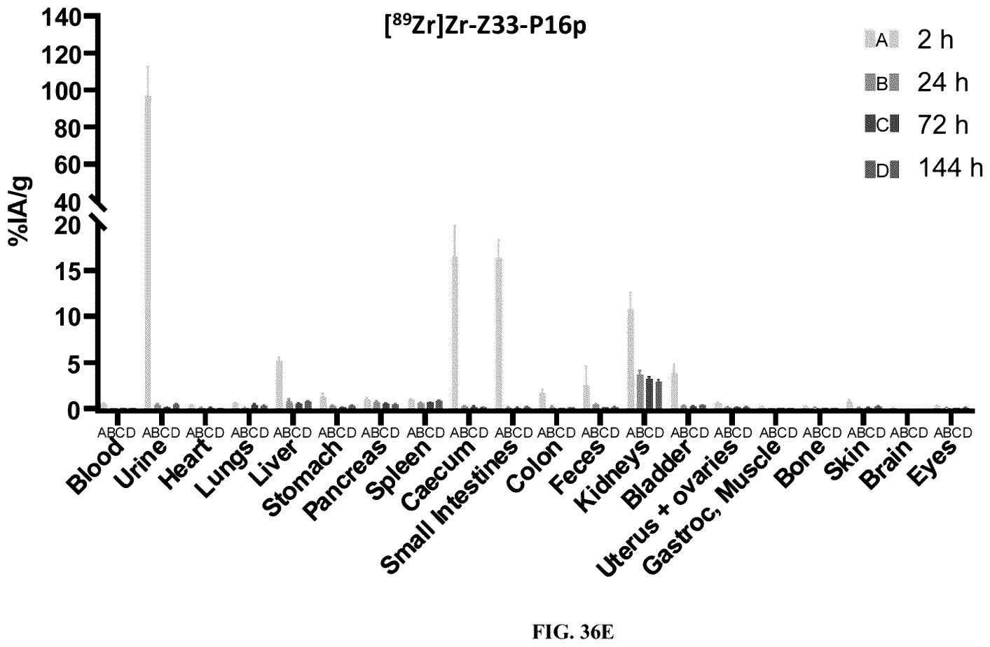

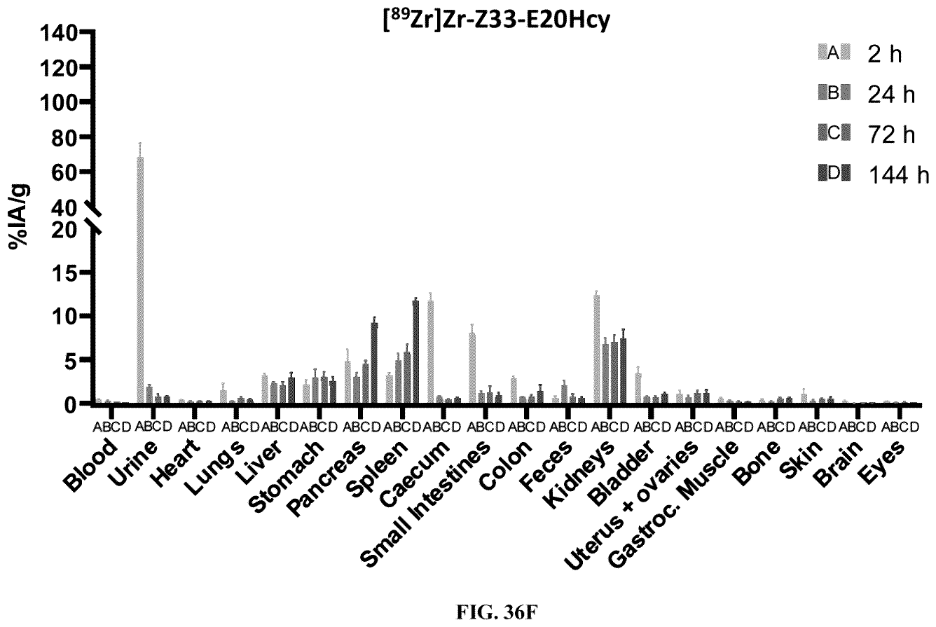

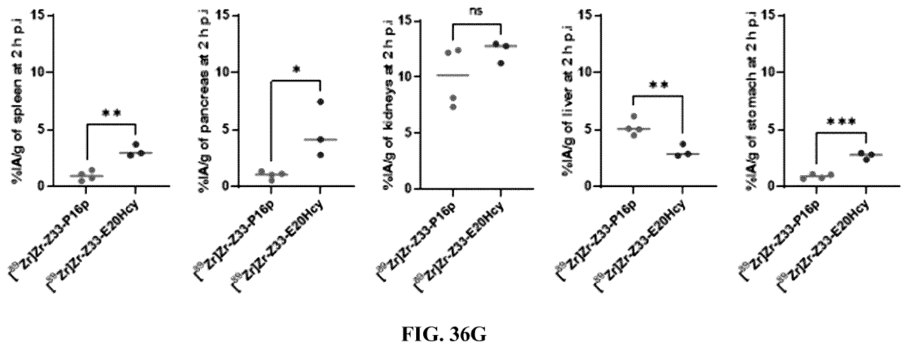

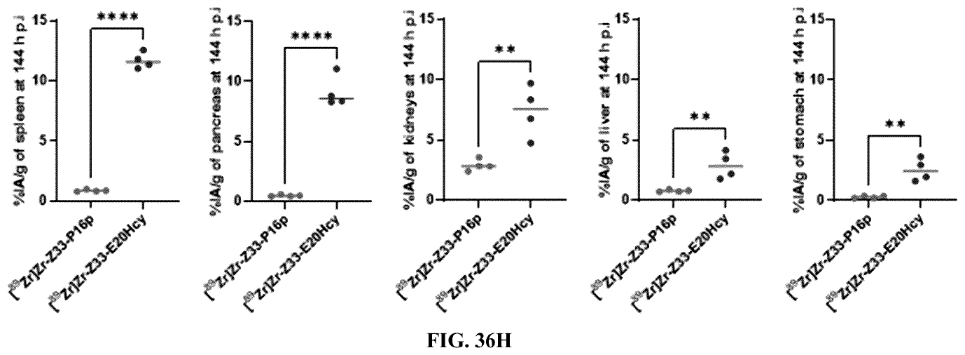

To evaluate the whole-body biodistribution in mice, Z33 peptides were radiolabeled. Z33-P16p and Z33-E20Hcy were both successfully conjugated to DFO and used for radiolabeling (FIGS. 36A to 36C). An experiment to determine the biodistribution and clearance mechanisms of radiolabeled Z33-peptides, the transfer of cargo to mouse IgG, and the length of detection after transfer was evaluated in a mouse model (FIG. 36D). Briefly, 16 mice were labeled with one of the Z33 peptides and at 2, 24, 72, and 144 hours post-intraperitoneal injection, 4 mice were sacrificed. Their organs were harvested and counted.

For both peptides, quick renal clearance was discovered. The control Z33-P16p was found to be cleared in about 2 hours, while the Z33-H20Hcy peptide was found to remain in specific organs for up to 144 hours post-injection (FIGS. 36E to 36F). Further, significant and persistent uptake was over 144 hours post-injection was found in tissues containing FcRn receptors, such as macrophages, monocytes, and dendritic cells. Uptake in the gut epithelium was also found (FIGS. 36G to 36H).

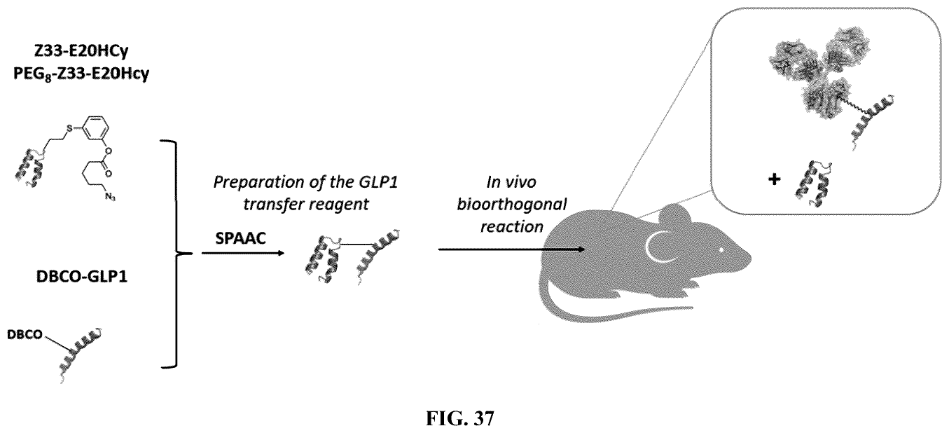

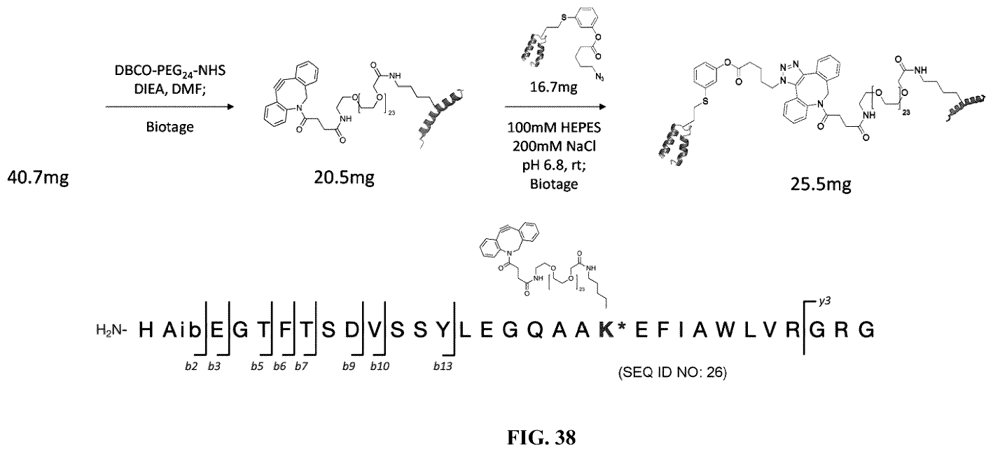

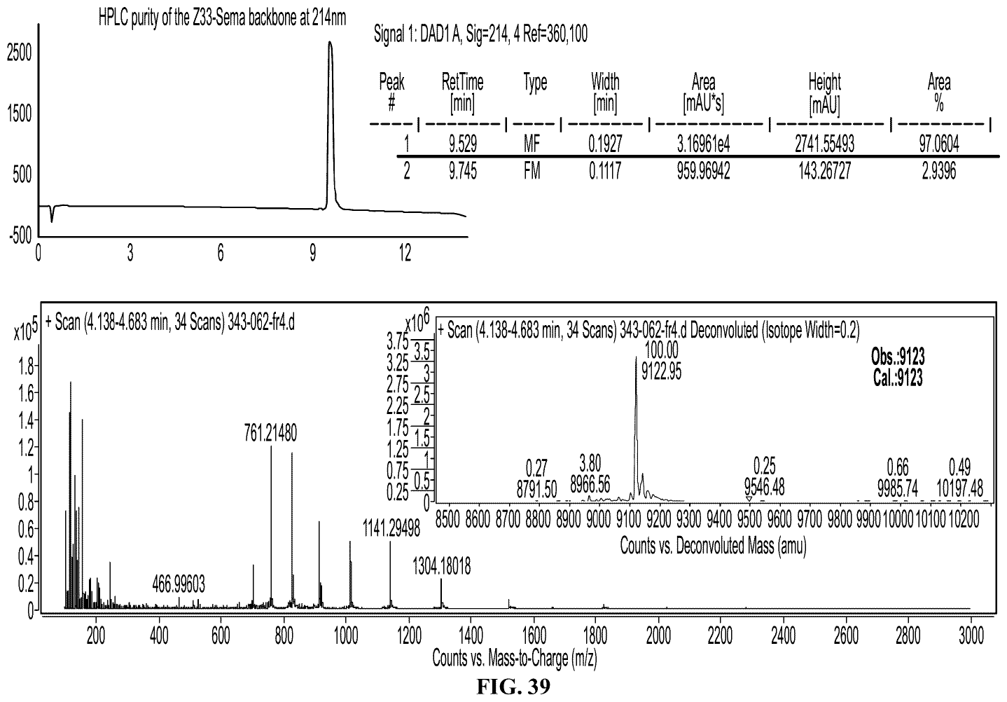

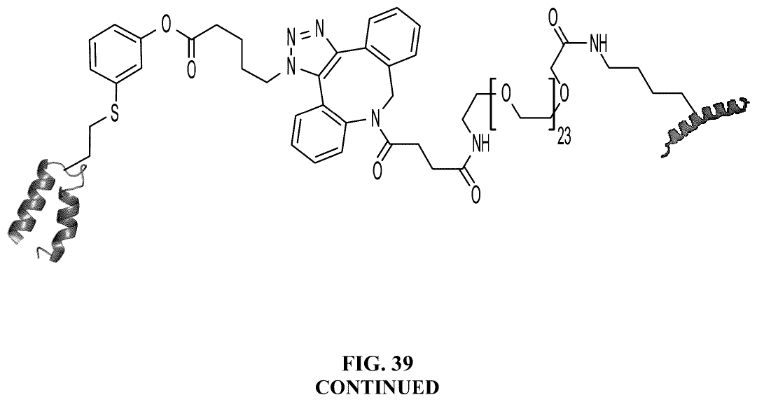

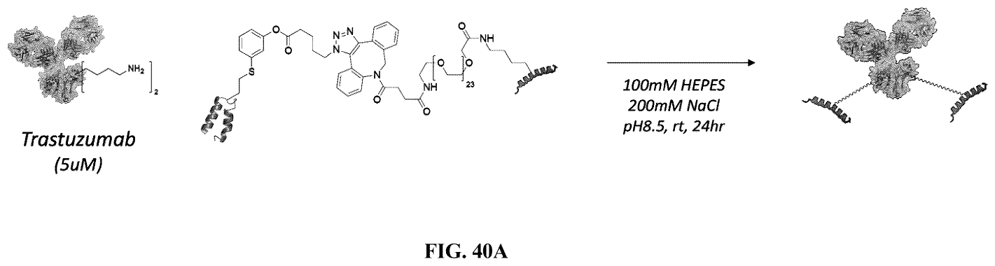

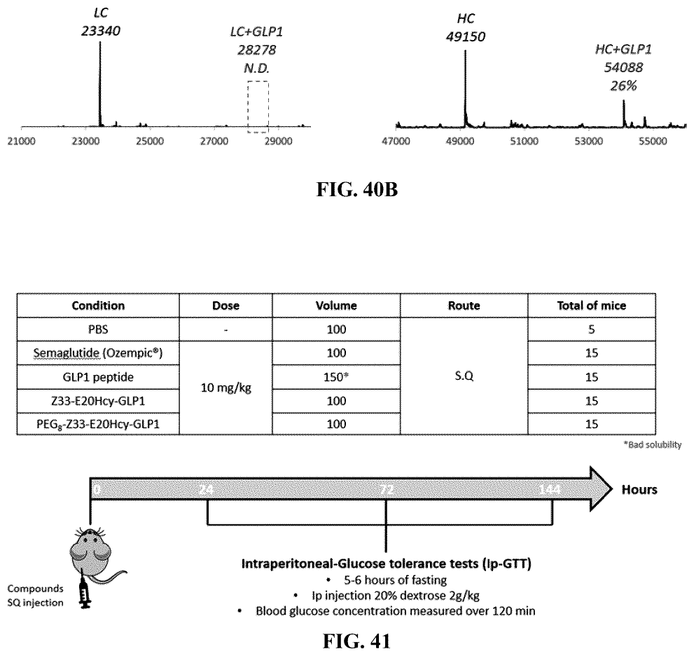

Finally, a proof-of-concept was developed to evaluate whether the PK/PD of GLP1 peptides using the Z33 transfer technology could be increased. An in vivo mouse model was set up (FIG. 37) as previously described. The GLP1 analog transfer reagent was synthesized from the lysine of the GLP1 analog (FIG. 38). Analysis of the purified reagent showed 97.1% purification using HPLC, and the pegylated compound was selected as a result of the reactivity screening (FIG. 39), which showed good reactivity and selectivity for IgG in vitro even with the bulky GLP1 (FIGS. 40A to 40B).

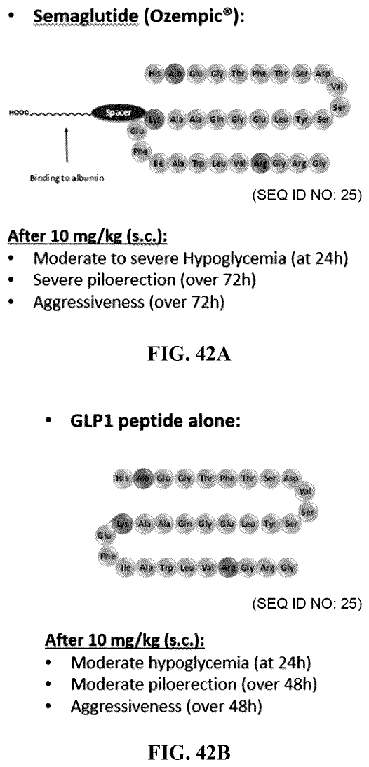

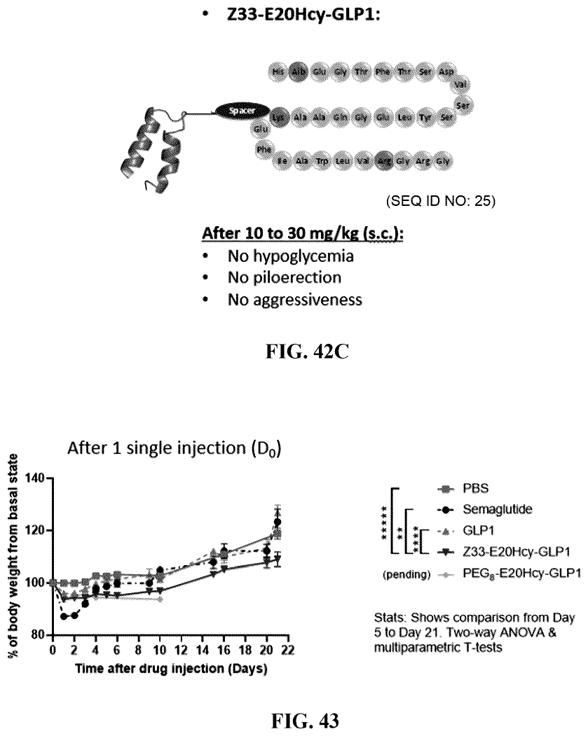

An in vivo experiment was set up, wherein mice were administered PBS, semaglutide, GLP1 peptide, Z33-E20Hcy-GLP1, or pegylated Z33-E20Hcy-GLP1 subcutaneously. At 24, 72, and 144 hours post-injection, an intraperitoneal glucose tolerance test (Ip-GTT) was performed with 20% dextrose (FIG. 41). The peptides evaluated are shown in FIGS. 42A to 42C. The maximum tolerated dose for semaglutide and GLP1 peptide alone was found to be 10 mg/kg. In contrast, Z33-E20Hcy-GLP1 and pegylated-Z33-E20Hcy-GLP1 showed no toxicity at 30 mg/kg. Body weight measurements showed that Z33-E20Hcy-GLP1 was effective for body weight management after a single injection (FIG. 43). Mice lost ˜5-7% body weight with Z33-GLP1 peptides for 10 days, in comparison to only 3 days with semaglutide. Further, the body weight gain was found to be inhibited for at least 21 days after the injection of Z33-E20Hcy-GLP1.

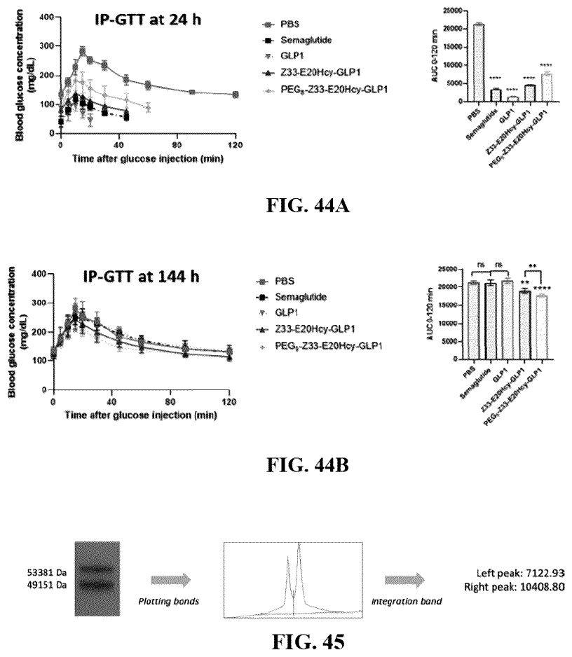

The pharmacodynamics of blood glucose management were also evaluated to determine for how long Z33-GLP1 peptide transfer reagents could regulate glucogenesis. Both Z33-GLP1 peptides were found to have an extended effect of around 6 days, whereas GLP1 alone and semaglutide were found to induce hypoglycemia (FIGS. 44A to 44B). The Z33-GLP1 peptides were found to work slower than the commercial semaglutide, but lasted up to 144 hours as opposed to only 72 hours. Finally, the pegylated Z33-E20Hcy0GLP1 was found to have better efficacy than the non-pegylated Z33.

Thus, the results demonstrate a biotechnology platform for in vivo biorthogonal site-selective modification of IgGs.

### REFERENCES FOR EXAMPLES 1 TO 13

1. Clin Cancer Res. 2004 Oct. 15; 10(20):7063-70.
2. Bioconjugate Chem. 2018, 29, 3522-3526.
3. Bioconjugate Chem. 2019, 30, 698-702.
4. Angew. Chem. Int. Ed. 2019, 58, 5592-5597.
5. J. Med. Chem. 2022, 65, 5751-5759.
6. Sci Rep. 2021 Nov. 18; 11(1):22521.
7. Nat Rev Drug Discov. 2021 April; 20(4):309-325.
8. Nature, 2015, 526, 687-6912.
9. Science, 2020, 368, 980-987.
10. Proc. Natl. Acad. Sci. U.S.A. 1996, 93 (June), 5688.
11. Org. Lett. 2017, 19, 4263-4266.
12. Proc. Natl. Acad. Sci. U.S.A.
13. 1996, 93 (June), 5688.
14. J. Am. Chem. Soc. 2016, 138, 12099-12111.
15. J. Am. Chem. Soc. 2018, 140, 3128-3133.
16. Physiol Rev, 2007, 87:1409-39.
17. Nat Rev Endocrinol, 2023, 19:201-216.
18. Nat Rev Endocrinol, 2012, 8:728-742.
19. Nature, 2023, 619:19.

### Example 14. In Vivo Antibody Painting for Next Generation Weight Loss Drugs

Peptide drugs are advantageous due to their high target selectivity, high efficacy, safety, and low cost (1-3). However, they also suffer from poor in vivo stability, short plasma half-life (a few minutes), and poor oral availability. Recent efforts in biotechnology focused on improving peptide pharmacokinetic (PK) and pharmacodynamic (PD) profiles using chemical modifications, to diminish renal clearance (4), increase chemical stability (5, 6), and enhance bioavailability (7).

One of the remarkable recent successes in the development of long-acting peptide drugs is exemplified by glucagon-like peptide-1 receptor agonists (GLP-1 RAs) (8-10). While native GLP-1 has a very short plasma half-life, of a few minutes, long-acting GLP-1 RAs have been proven efficient for the treatment of type II diabetes mellitus (T2DM) and obesity (11-16). GLP-RAs conjugation to neonatal Fc receptor (FcRn) binders, such as serum albumin or IgGs, leads to endosomal internalization, transportation and recycling to the blood (17, 18). Despite great achievements, the first generations of long-acting GLP-1 RAs are associated with high costs for IgG production and purification. New technologies that leverage native antibodies, obviate in vitro IgG production, and enable the assembly of the IgG-GLP-1 conjugate in vivo would greatly streamline the manufacturing process and reduce costs of next-generation long-acting GLP-1 RAs.

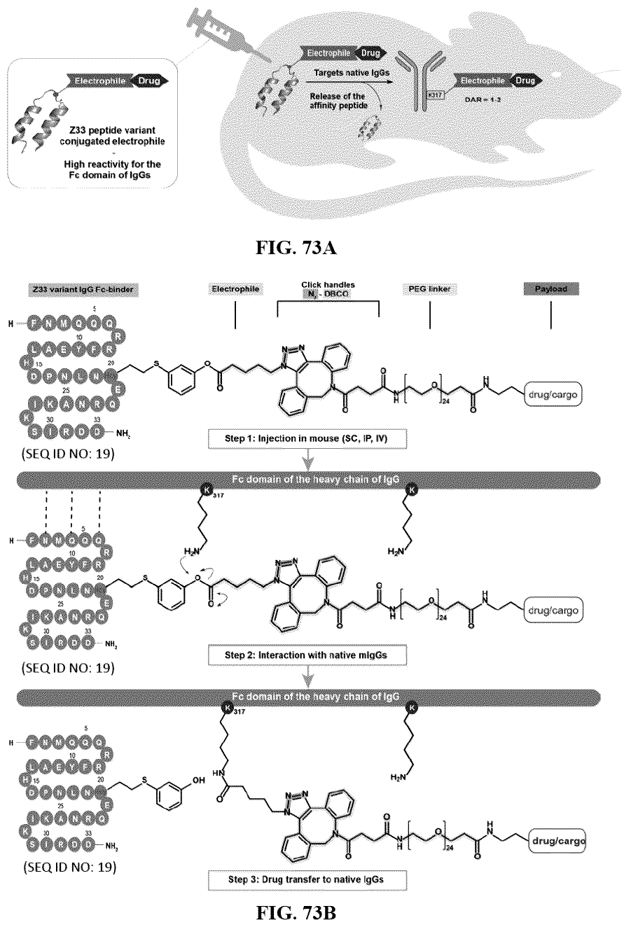

Here, a novel drug delivery platform to attach therapeutic payloads specifically to IgGs directly in vivo, aiming to extend the PK/PD of both existing and candidate drugs via FcRn binding, is described. The technology is at times referred to as “in vivo antibody painting” as different lysine (Lys) residues from the Fc domain of native IgGs can be subsequently modified site-selectively to incorporate multiple drugs (FIGS. 73A and 73B). To achieve specific targeting of IgGs, several electrophilic affinity peptides composed of three parts: (i) an Fc-binder peptide, (ii) a reactive electrophilic function, and (iii) a therapeutic payload, were designed. Through proximity-induced effect (19), the electrophilic payload is covalently conjugated to the heavy chain of the IgG Fc domain. The reaction is biocompatible, occurs at physiological pH and temperature (37° C.) without any catalyst, and does not require incorporation of any reactive handle to IgGs beforehand.

Fc-binder affinity peptides were synthesized by Fmoc-based solid-phase peptide synthesis (Fmoc-SPPS) using previously reported automated fast-flow peptide synthesis (AFPS) instrumentation (20). Their safety and duration of efficacy was assessed in two different mice models, WT lean Swiss and Lep^{ob/ob}, the latter having spontaneous type II diabetes mellitus (T2DM) related obesity. The results show promising outcomes for advancing the next generation of weight loss and T2DM drugs. Furthermore, the applicability of IgG painting to a variety of payloads (small molecules, peptides, and radionuclides) is demonstrated, highlighting its versatility and potential for numerous applications. This work thereby represents a novel impactful insight for designing long-acting drugs and antibody-drug conjugates.

### Synthesis of Fc-Reactive Derivatives of the Minimal Z-Domain of Protein A

The minimal Z-domain of protein A, a 33-mer peptide (Z33), was shown to bind the Fc fragment of IgG1 with an apparent dissociation constant (K_{D}) of 43 nM (21). In a previous study, it was determined that specific amino acids in the sequence of the Z33 peptide have little to no influence on its binding to IgGs while other residues play a crucial role (22). The interaction between Glu-20 (E20) of protein A and Lys-317 (K317, (23, 24)) of the Fc domain of IgG1 was previously confirmed using co-crystal structure (FIG. 77). Selective targeting of the Fc fragment of native IgGs, using affinity peptides, was already successfully reported for manufacturing antibody-drug conjugates (ADCs), via the AJICAP® technology (25, 26). However, the first generation required redox treatment for reagent conjugation to thiol groups and was thereby associated to high risks of aggregation. While the second generation brought significant improvements, and enabled to maintain aggregation under 10%, IgG modification was still achieved following multi-steps chemical reactions, making the technology non-suitable for direct in vivo targeting (27). Following this evidence, a novel drug delivery platform was developed, derived from Z33 peptide, for targeting and modifying native IgGs directly in vivo, in one single step. Here, it is expected that i) aggregation and immunogenicity can be avoided by using fully biocompatible reagents, ii) the half-life of peptide drugs can be increased, and iii) lower doses can be administered.

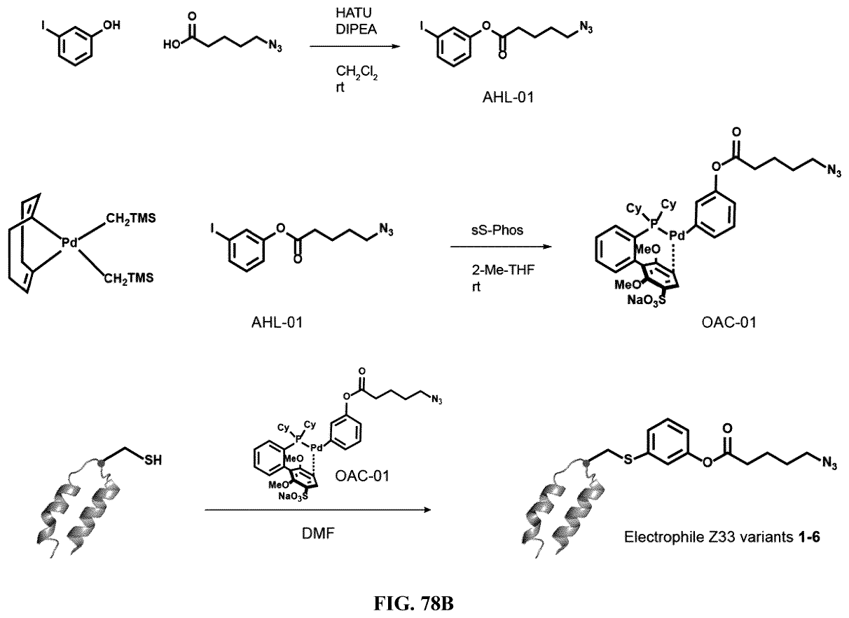

First several variants of Z33 were designed and synthesized using previously reported AFPS technology (FIG. 78A and Table 16) (20). Based on previous findings (22), E20 was substituted with either L-Cys (peptide 1), or L-homocysteine (L-Hcy, peptides 2-6) to introduce a handle for the site-specific conjugation of an electrophile function bearing a free azido click moiety. The azido electrophile was conjugated by S-arylation using Pd-oxidative addition complexes (Pd-OAC) (28), leading to the final electrophile-containing affinity peptide constructs (N₃-1-6, FIG. 78B). N₃-1 was used as a negative control as the L-Pro-16 substitution with a D-Pro resulted in a loss of binding affinity (˜3 μM). N₃-3 was based on the same structure as N₃-2 (binding affinity ˜60 nM) except its N-terminus is capped using 8 repeated units of polyethylene glycol (PEG₈).

TABLE 16
Sequences and mass of the synthesized Z33 variants.

| | | | | Calcul | |
|---|---|---|---|---|---|
| | | SEQ | | ated | Observed |
| | | ID | | Mass | Mass |
| Code | Sequence | NO: | Electrophile | (Da) | (Da)^{c} |
| N₃-1-I | FNMQQQRRFYEALHD pNLNCEQRNAKIKSI RDD | 20 | 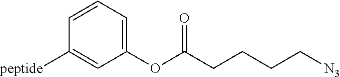 | 4295.8 | 4296.1 |
| N₃-2-I | FNMQQQRRFYEALHD PNLNHcyEQRNAKIKSI RDD | 19 | 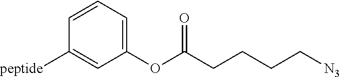 | 4309.8 | 4310.0 |
| N₃-3-I | H2N-PEG8- FNMQQQRRFYEALHD PNLNHcyEQRNAKIKSI RDD | 19 | 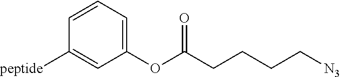 | 4733.3 | 4733.3 |
| N₃-4-I | FNMQQQRRFYEALHD PNLNHcyEQRNAKIKSI RDd | 18 | 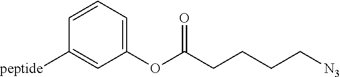 | 4309.8 | 4309.7 |
| N₃-5-I | fNMQQQRRFYEALHD PNL NHcyEQRNAKIKSI RDd | 28 | 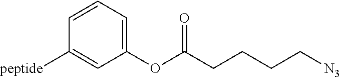 | 4309.8 | 4309.7 |
| N₃-6-I | fNMQQQRRFYEALHD PNLNHcyEQRNAKIKSI Rdd | 29 | 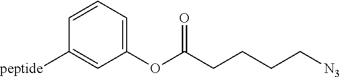 | 4309.8 | 4309.7 |
| N₃-7- VI | FNCQQQRRFYEALHD PNLNEEQRNAKIKSI RDD | 12 | 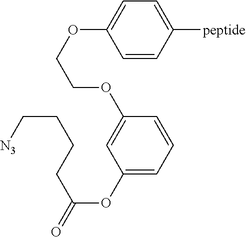 | 4429.9 | 4430.1 |
| N₃-8- VI | FNHcyQQQRRFYEALH D PNLNEEQRNAKIKSI RDD | 22 | 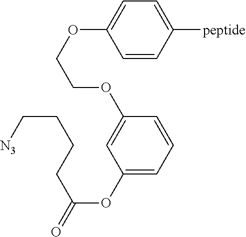 | 4443.9 | 4443.8 |
| N₃-7- VII | FNCQQQRRFYEALHD PNLNEEQRNAKIKSI RDD | 12 | 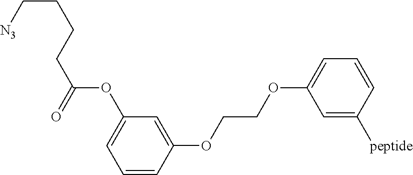 | 4429.9 | 4430.6 |
| N₃-8- VII | FNHcyQQQRRFYEALH D PNLNEEQRNAKIKSI RDD | 22 | 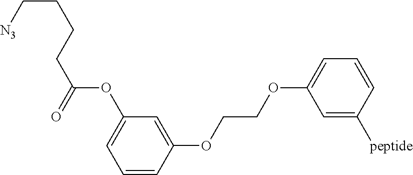 | 4443.9 | 4444.4 |
| N₃-9- VII | FNMQQQRRFYEALHD PNLNEEQRNAKIKSI CDD | 17 | 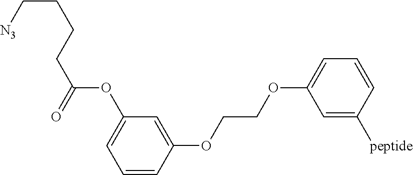 | 4404.9 | 4405.8 |
| N₃-10- VII | FNMQQQRRFYEALHD PNLNEEQRNAKIKSI RCD | 18 | 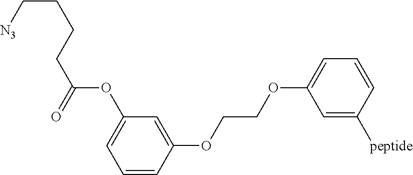 | 4446.0 | 4445.7 |
| N₃-11- VII | FNMQQQRRFYEALHD PNLNEEQRNAKIKSI RHcyD | 21 | 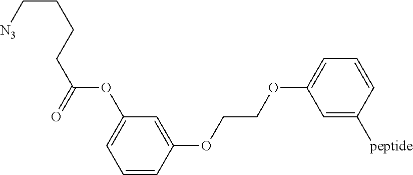 | 4460.0 | 4460.2 |
| N₃-12- VIII | FNMQQQRRFYEALHD PNLNHcyEQRNAKIKSI RDD | 19 | 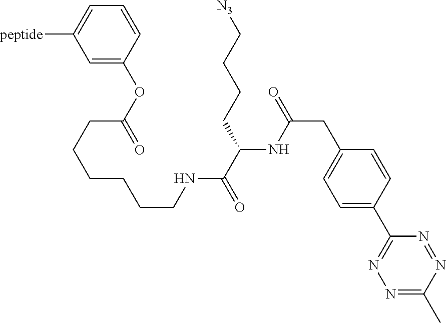 | 4678.1 | 4678.5 |
| N₃-13- VIII | H2N-PEG8- FNMQQQRRFYEALHD PNLNHcyEQRNAKIKSI RDD | 19 | 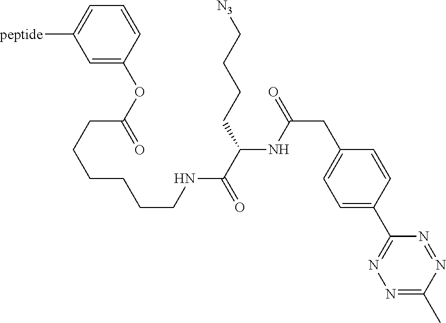 | 5101.8 | 5102.2 |

In vitro selective transfer of azido moieties onto human and mouse IgGs

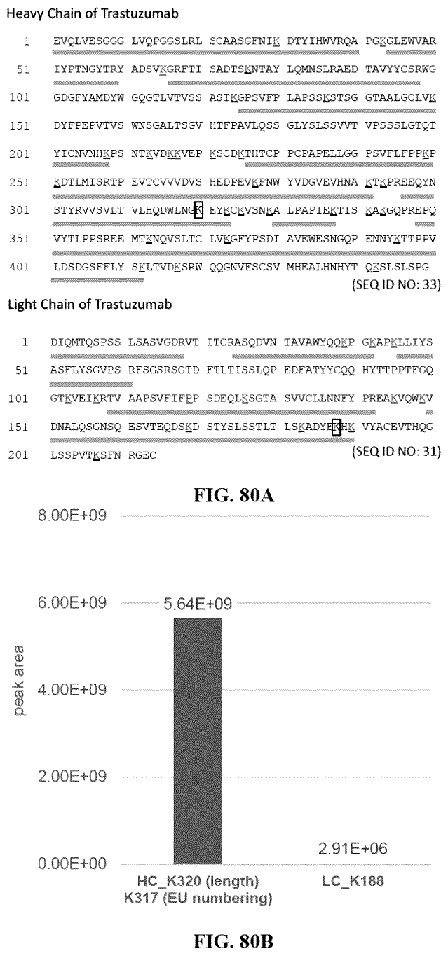

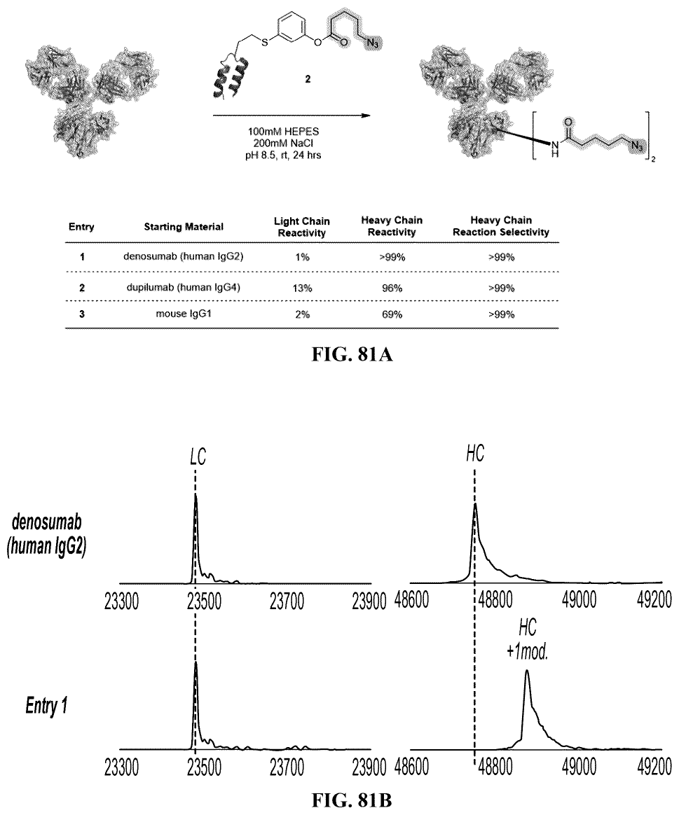

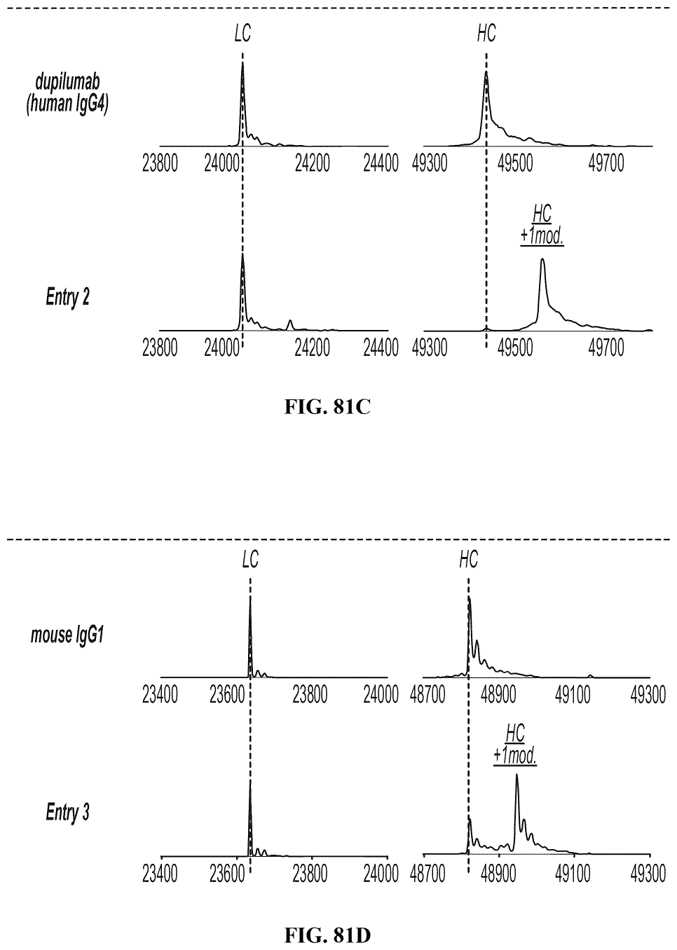

Several electrophiles (I, II, III, IV, V) were synthesized, and their reactivity after 24 h was assessed in vitro, using hIgG1 trastuzumab (Tmab; 1 eq.) (FIGS. 79A and 79B). The highest reaction transfer was observed for electrophile I, with up to 99% of modifications incorporated into the heavy chain of Tmab after reaction with peptide 2 (20 eq.) compared to only 47% with electrophiles II or III. Through a sequencing experiment using tandem liquid chromatography-mass spectrometry (LC-MS/MS), it was identified that the electrophile moiety was covalently transferred to the Lys-317 side chain of the heavy chain of the Fc domain of Tmab (FIGS. 80A and 80B). Further investigations showed conversions of ˜17%, 99%, and 95%, after reaction with peptides N₃-1-3 bearing electrophile I, respectively, confirming the highest reactivity associated with N₃-2 (Table 17). In vitro reactivity of peptide N₃-2-I (20 eq.) was then assessed in vitro for other IgG subtypes and species and showed the highest reactivity was achieved using hIgG1-2 and excess amount of N₃-2-I, with 98% conversion on the heavy chain Fc domain associated to a high selectivity with less than 2% of the light chains modified with the electrophile (FIGS. 81A to 81D and Table 18).

TABLE 17
Quantification of azido transfer on pure human IgG1 Trastuzumab.

| | | | | | | Result analyzed by Q-ToF LC-MS | | |
|---|---|---|---|---|---|---|---|---|
| | | | | | | Light | Heavy | Heavy |
| | | | | | | chain | chain | chain total |
| | | | Equivalent | Re- | Re- | total | total | conversion |
| | Electro- | | of | action | action | con- | con- | ratio |
| Peptide | phile | R | reagent | buffer | Temp | version | version | (+1/+2) |
| N₃-1 | I | 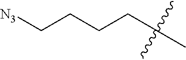 [SMILES: *CCCCN=[N+]=[N-]] | 10 | 100 mM HEPE S pH 8.5 | rt | <1% | 17% | >99%/<1% |
| N₃-1 | I | 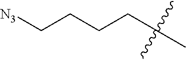 [SMILES: *CCCCN=[N+]=[N-]] | 10 | PBS | rt | <1% | 12% | >99%/<1% |
| N₃-2 | I | 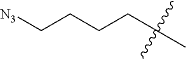 [SMILES: *CCCCN=[N+]=[N-]] | 10 | 100 mM HEPE S pH 8.5 | rt | 5% | 99% | >99%/<1% |
| N₃-2 | I | 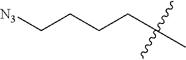 [SMILES: *CCCCN=[N+]=[N-]] | 20 | PBS | rt | 12% | 68% | 88/12 |
| N₃-3 | I | 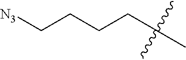 [SMILES: *CCCCN=[N+]=[N-]] | 10 | 100 mM HEPE S pH 8.5 | rt | 5% | 95% | 95/5 |
| N₃-3 | I | 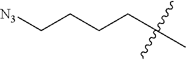 [SMILES: *CCCCN=[N+]=[N-]] | 10 | PBS | 37 | 4% | 86% | 95/5 |
| N₃-12 | I | 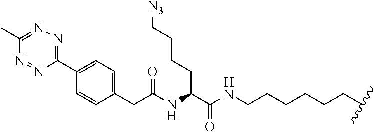 [SMILES: *CCCCCCNC(=O)[C@H](CCCCN=[N+]=[N-])NC(=O)Cc1ccc(-c2nnc(C)nn2)cc1] | 10 + 10^{a} | 100 mM HEPE S pH 8.7 | 37 | <1% | 84% | >99%/<1% |

^{a}10 equivalents of the reagent were added at the beginning and after 4 hours of reaction, respectively.

TABLE 18
Quantification of azido transfer on different pure human and mouse IgG subtypes.

| | | | | | Result analyzed by Q-ToF LC-MS | | |
|---|---|---|---|---|---|---|---|
| | | | | | Light | Light | Heavy chain |
| | | | Equivalent | Reaction | chain total | chain total | total conversion |
| IgG | Peptide | Electrophile | of reagent | temp. | conversion | conversion | ratio (+1/+2) |
| Denosumab | N₃-2 | I | 20 | rt | 1% | >99% | >99%/<1% |
| (hIgG2) | | | | | | | |
| Dupilumab | N₃-2 | I | 20 | rt | 13% | 96% | >99%/<1% |
| (hIgG4) | | | | | | | |
| Mouse IgG1 | N₃-2 | I | 20 | rt | 2% | 69% | >99%/<1% |

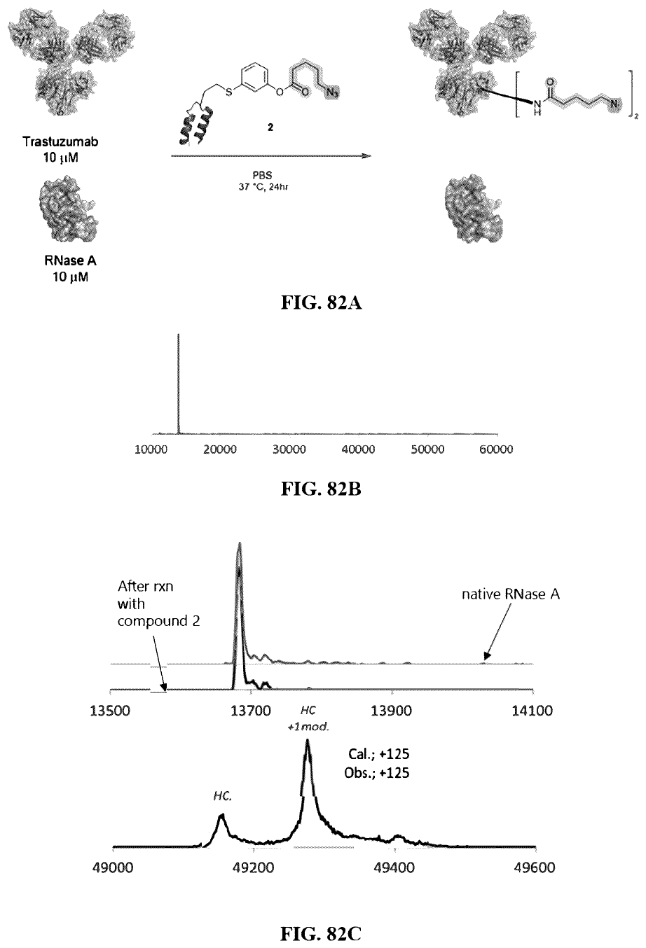

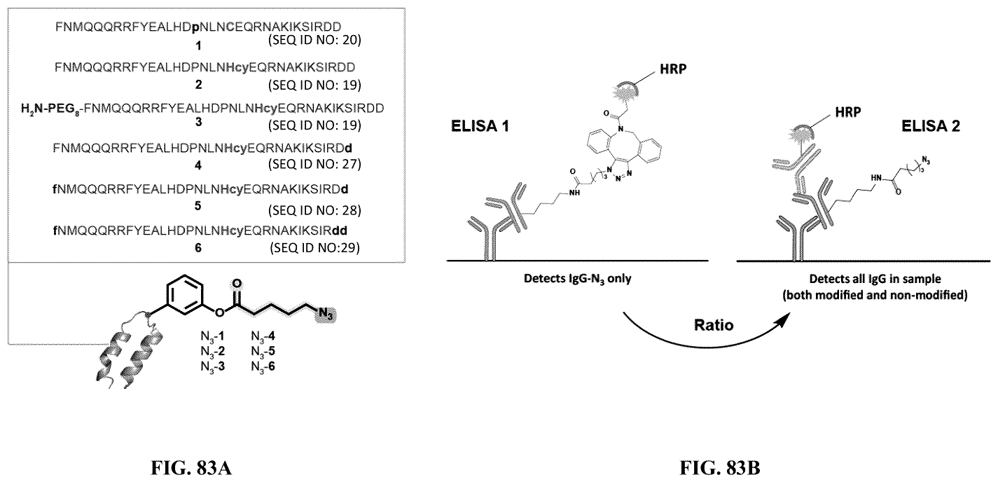

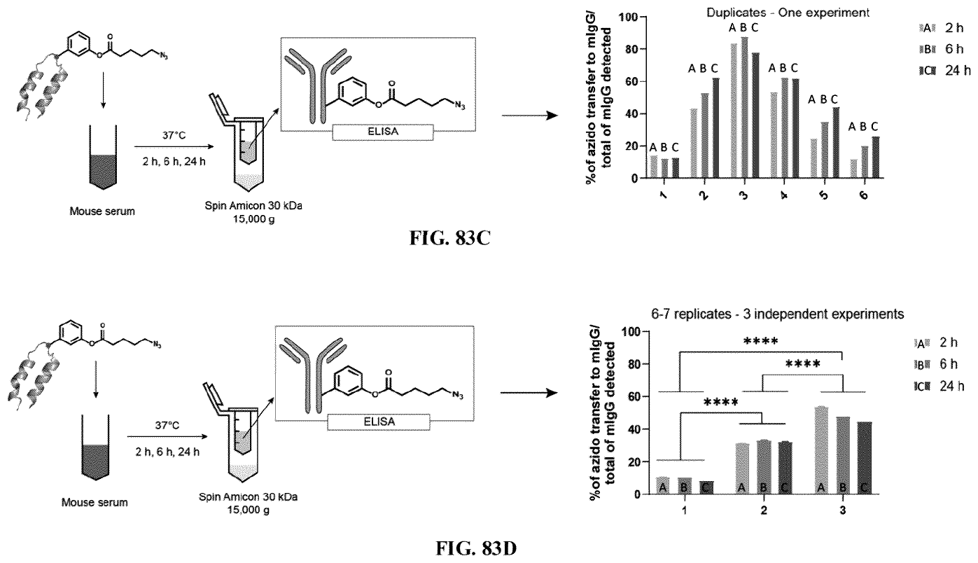

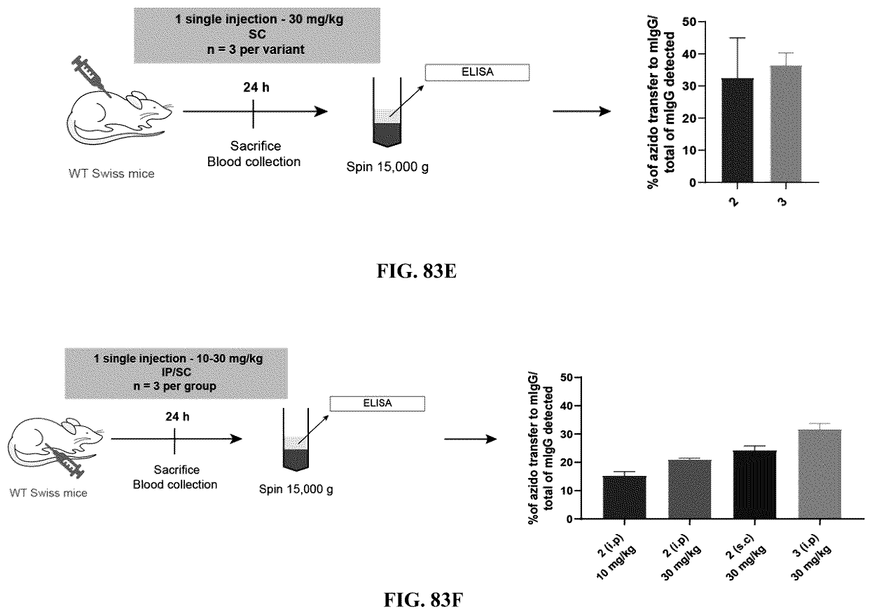

Peptide N₃-2-I (20 eq.) incubated with a mixture of Tmab (1 eq.) and RNase A (1 eq.) showed that only Tmab ended being decorated with azido moieties (DAR 1-2) (FIGS. 82A to 82C). In addition, incubation of N₃-1-6-I in mouse sera confirmed the absence of binding to other biomolecules circulating in blood (FIGS. 83A to 83D). The highest IgG conversion rate was achieved using N₃-2-I and N₃-3-I, with 33±1% and 49±3%, respectively, after 2 h of incubation, versus 10±1% for the low binder N₃-1-I Given these good conversion rates, N₃-2,3-I were taken forward for investigations in WT Swiss mice (FIGS. 83E to 83F). It was calculated that at least 30 mg/kg of N₃-2,3 (˜280 nmol) would be required to modify ˜100% of the mIgGs circulating in blood (˜3 mg/mL of mIgGs), with a DAR of at least 1 and a conversion rate of 100% (29, 30). However, as N₃-2-3 only induced up to 50% of electrophile transfer, in vitro, only 50% of the mice native IgGs in vivo were expected to be modified, at best, after a single dose of 30 mg/kg. The dose of 30 mg/kg of N₃-2-3-I was well tolerated after either subcutaneous (SC) or intraperitoneal (IP) injection, no significant adverse effects being observed. N₃-2-I and N₃-3-I showed similar in vivo reactivity, with 33±12% and 37±4% conversion rate, respectively, after SC injection. Considering that the two heavy chain Lys-317 side chains of the Fc domain can be modified using N₃-2-3-I reagents, the painting of mIgGs resulted in a distribution of DAR from 0 to 2. This methodology does not enable to quantify how many modifications are carried on mIgG; therefore, it is assumed that the conversion rate quantified here is probably underestimated.

In Vitro Selective Transfer of GLP-1 Conjugates onto Mouse IgGs

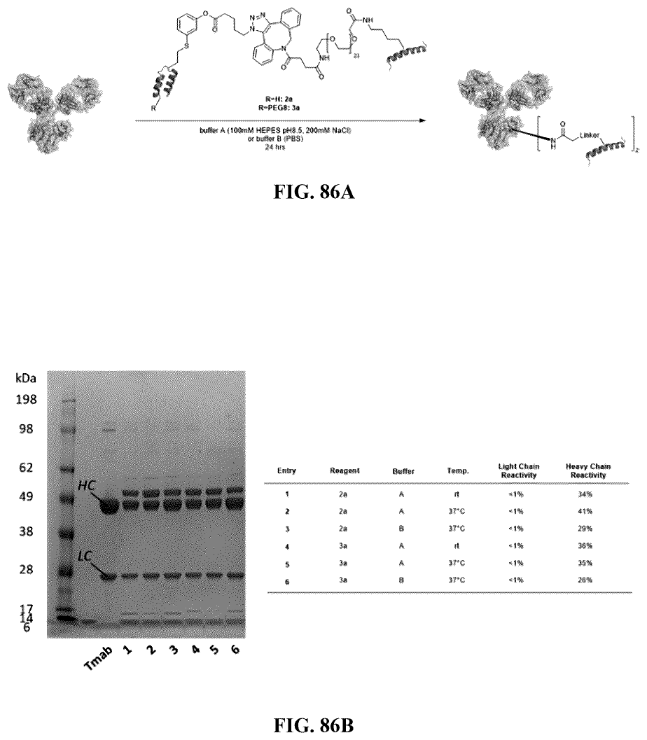

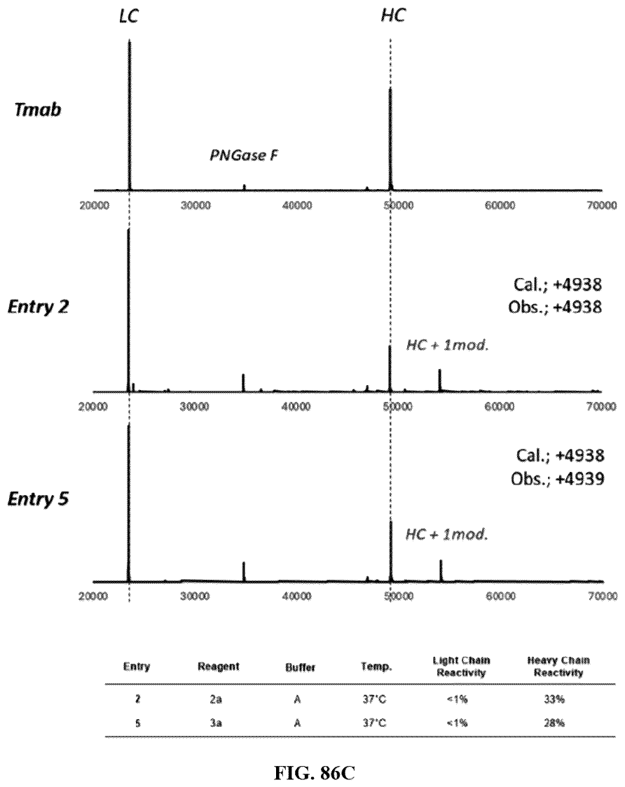

Motivated by the encouraging results obtained with azido transfer, the IgG painting technology was applied to larger biomolecular cargos, such as GLP-1 RAs for extending their PK. Three different GLP-1 backbones were synthesized using AFPS (FIGS. 84A and 84B), based on the sequences of semaglutide (GLP-1a), liraglutide (GLP-1b), and tirzepatide (GLP-1c). GLP-1a,b were covalently conjugated to a dibenzyl cyclooctyne (DBCO) moiety on Lys-20 (FIGS. 84A to 88B) then reacted with N₃-2-I to obtain electrophile peptides 2a,b through the strain-promoted azide-alkyne cycloaddition click reaction (SPAAC) (31). Electrophile peptide 2c was obtained following a multiple step conjugation process (FIG. 84B). The conversion rate of peptides 2a-c determined by Q-ToF LC-MS indicated that up to 41% of Tmab Fc domain heavy chains were successfully modified with GLP-1a, 26% for GLP-1b, and 20% with GLP-1c (FIGS. 86A to 88B, and Table 19). Following these encouraging results, the effect of in vivo IgG painting with peptides 2a,3a in vivo was assessed in two different mice models.

TABLE 19
Quantification of GLP-1a transfer
on pure human IgG₁ Trastuzumab.

| | | | | Result analyzed by Q-ToF LC-MS | | |
|---|---|---|---|---|---|---|
| | | | | | | Heavy |
| | | | | | | chain |
| | | | | Light | Heavy | total |
| | Equiv- | | Reac- | chain | chain | conversion |
| Pep- | alent of | | tion | total | total | ratio |
| tide | reagent | Buffer | Temp. | conversion | conversion | (+1/+2) |
| 2a | 20 | HEPES | rt | <1% | 27% | >99%/<1% |
| 2a | 20 | HEPES | 37° C. | <1% | 33% | 94/6 |
| 3a | 20 | HEPES | rt | <1% | 26% | >99%/<1% |
| 3a | 20 | HEPES | 37° C. | <1% | 28% | >99%/<1% |
| 3a | 20 | PBS | 37° C. | <1% | 16% | >99%/<1% |

### In Vivo IgG Painting is Well Tolerated in Mice

Three different doses of the commercial semaglutide (0.5 mg/kg (˜5 nmol), 3 mg/kg (˜30 nmol) or 10 mg/kg (˜100 nmol)) were first SC injected in WT mice to verify the tolerability for GLP-1 RAs (Table 20). In the other cohorts, 2a,3a was SC administered following either 10 mg/kg (25 nmol), 30 mg/kg (75 nmol), or three injections of 10 mg/kg, 1 per week for 3 weeks (30 mg/kg total, 75 nmol total). As expected, hypoglycemia was observed starting 0.5 mg/kg of semaglutide and became critical for the highest dose suggesting 10 mg/kg as the maximum tolerated dose. None of the mice receiving SC injections of 2a,3a showed hypoglycemia or behavioral change, confirming the safety of IgG painting with GLP-1 RAs.

In Lep^{ob/ob} obese mice, two routes were assessed for the 10 mg/kg dose: SC and IP (Table 21). For Semaglutide, both routes did significantly impact the mice behavior and induce hypoglycemia. In the 2a,3a cohorts, the SC route was better tolerated with no significant toxicity while IP induced some behavioral changes. Investigations about the PD of 2a,3a following SC injection of 10 mg/kg were pursued in both mice models.

TABLE 20
Safety profile of GLP-la conjugates in female WT Swiss mice. All GLP-1a drugs were injected
subcutaneously. Every cohort contains 15 mice total, except for semaglutide at 0.5 mg/kg
and 3 mg/kg, and 2a stacking dose (3 × 10 mg/kg) which contain 5 mice each. Measured
mean fasting blood glucose in naive female WT Swiss mice: 134 ± 10 mg/dL.

| | | | Number of mice with | | | | | | | |
|---|---|---|---|---|---|---|---|---|---|---|
| | | Mean | Hypoglycemia* | | | | | | Number of | |
| | | fasting | Mild | Moderate | Extreme | Number of mice with | | | mice showing | |
| | Dose | glucose | (40-60 | (20-40 | (≤20 | Piloerection | | | significant | |
| Compound | (mg/kg) | (mg/dL)* | mg/dL) | mg/dL) | mg/dL) | Mild | Moderate | Severe | aggressiveness | MTD |
| Semaglutide | 0.5 | 60.8 ± | 2 | — | — | — | — | — | — | 10 |
| | | 9.4 | | | | | | | | mg/kg |
| | 3 | 61.4 ± | 1 | 1 | — | — | 2¤ | 3¤ | 5¤ | |
| | | 13.5 | | | | | | | | |
| | 10 | 40.4 ± | 1 | 2 | 1 | — | — | 15£ | 15£ | |
| | | 12.1 | | | | | | | | |
| GLP-1a | 10 | 40.8 ± | — | 3 | 1 | — | 15¤ | — | 15¤ | 10 |
| | | 11.8 | | | | | | | | mg/kg |
| 2a | 10 | 81.8 ± | — | — | — | — | — | — | — | ≥30 |
| | | 9.4 | | | | | | | | mg/kg |
| | 3 × 10, | 93.2 ± | — | — | — | — | — | — | — | |
| | over 3 | 11.4 | | | | | | | | |
| | weeks | | | | | | | | | |
| | 30 | 94.0 ± | — | — | — | 2* | — | — | — | |
| | | 4.7 | | | | | | | | |
| 3a | 10 | 82.4 ± | — | — | — | — | — | — | — | >10 |
| | | 9.7 | | | | | | | | mg/kg |

*At 24 h post drug injection
¤Over 48 h post injection
£Over 72 h post injection
MTD: Maximum tolerated dose among the assessed range.

TABLE 21
Safety profile after 10 mg/kg of GLP-1a conjugates in male (M) and female (F) Lep^{ob/ob} mice.
IP: intraperitoneal injection. SC: subcutaneous injection. Cohorts of 3 mice per row. Measured
mean fasting blood glucose in female C57BL/6J mice: 174 ± 15 mg/dL; male C57BL/6J mice: 184 ±
10 mg/dL; female Lep^{ob/ob} mice: 374 ± 29 mg/dL; male Lep^{ob/ob} mice: 407 ± 44 mg/dL.

| | | Mean | Number of mice with | | | | | | |
|---|---|---|---|---|---|---|---|---|---|
| | | fasting | Hypoglycemia* | | | | | | Number of |
| | Sexe and | blood | Mild | Moderate | Extreme | Number of mice with | | | mice showing |
| | route of | glucose | (40-60 | (20-40 | (≤20 | Piloerection | | | significant |
| Compound | injection | (mg/dL)* | mg/dL) | mg/dL) | mg/dL) | Mild | Moderate | Severe | aggressiveness |
| Semaglutide | F, IP | 104 ± 6 | — | — | — | — | — | 3 | 3 |
| | F, SC | 97 ± 7 | — | — | — | — | 3 | — | 3 |
| | M, IP | 111 ± 14 | — | — | — | — | — | 3 | 3 |
| | M, SC | 75 ± 12 | 2 | — | — | — | 3 | — | 3 |
| 2a | F, IP | 137 ± 8 | — | — | — | 2 | 1 | — | — |
| | F, SC | 104 ± 5 | — | — | — | — | — | — | — |
| | M, IP | 149 ± 32 | — | — | — | — | 3 | — | 1 |
| | M, SC | 108 ± 4 | — | — | — | 1 | — | — | — |
| 3a | F, IP | 147 ± 35 | — | — | — | 1 | — | — | — |
| | F, SC | 130 ± 12 | — | — | — | — | — | — | — |
| | M, IP | 167 ± 22 | — | — | — | 2 | — | — | — |
| | M, SC | 102 ± 9 | — | — | — | - | — | — | — |

The Pharmacodynamic Profile of GLP-1 is Significantly Improved after In Vivo IgG Painting in Lean Mice

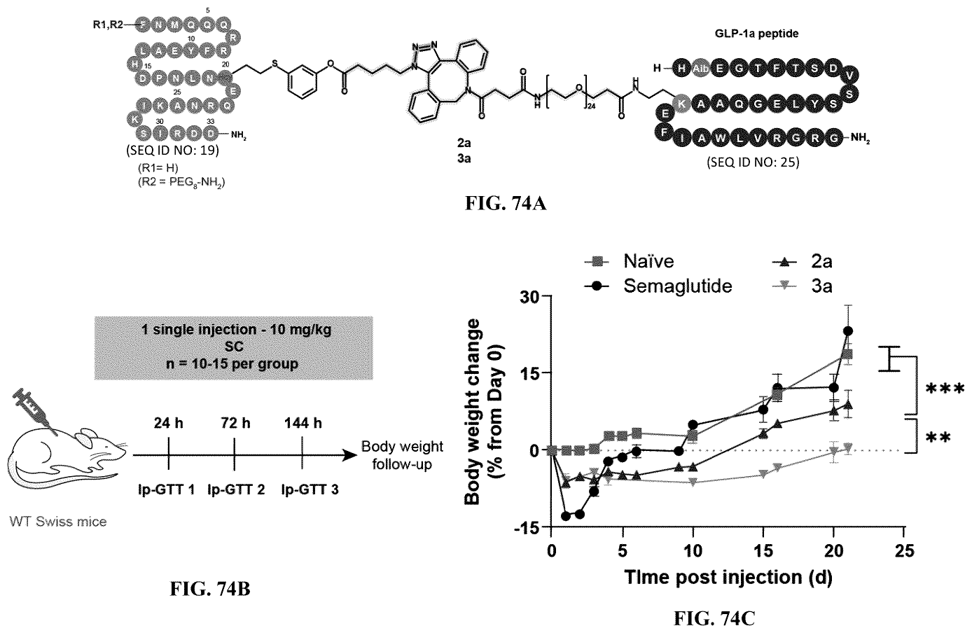

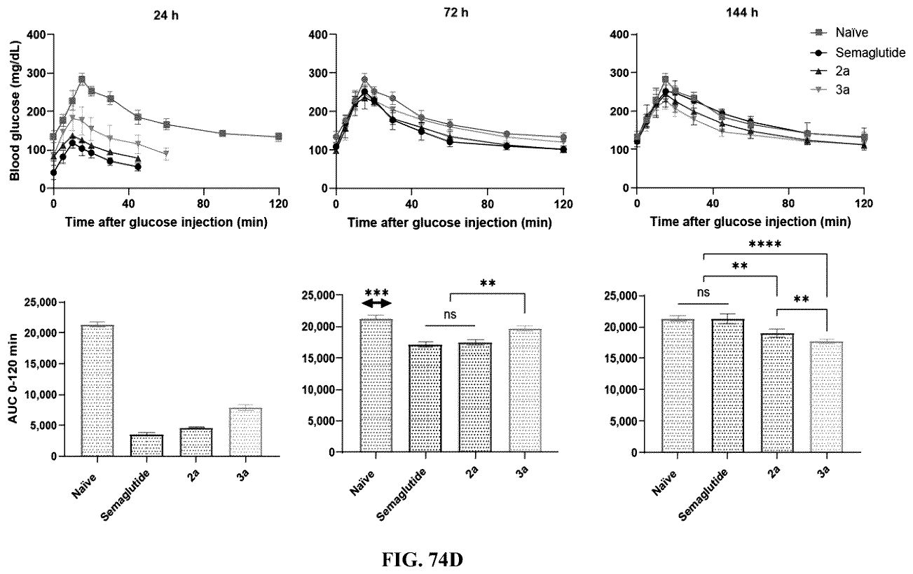

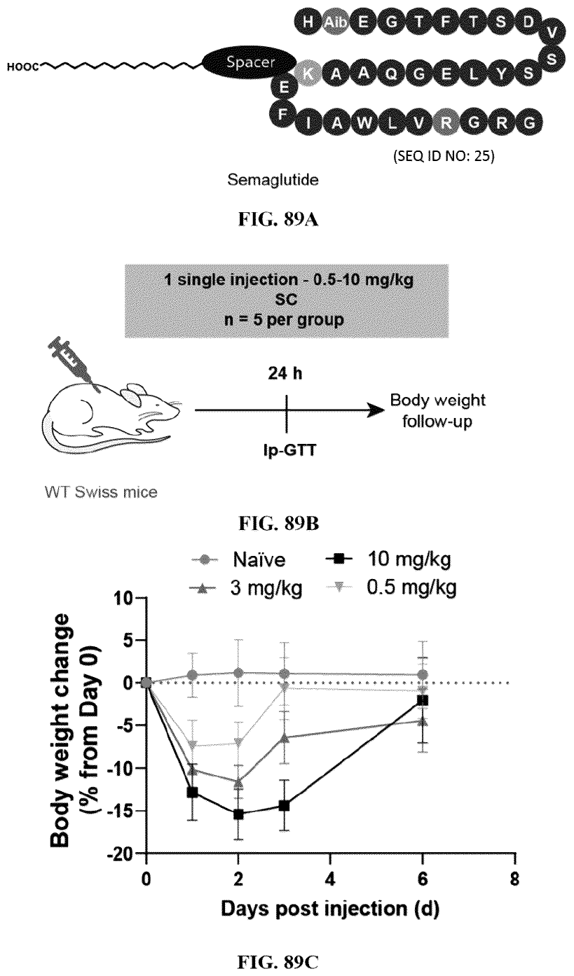

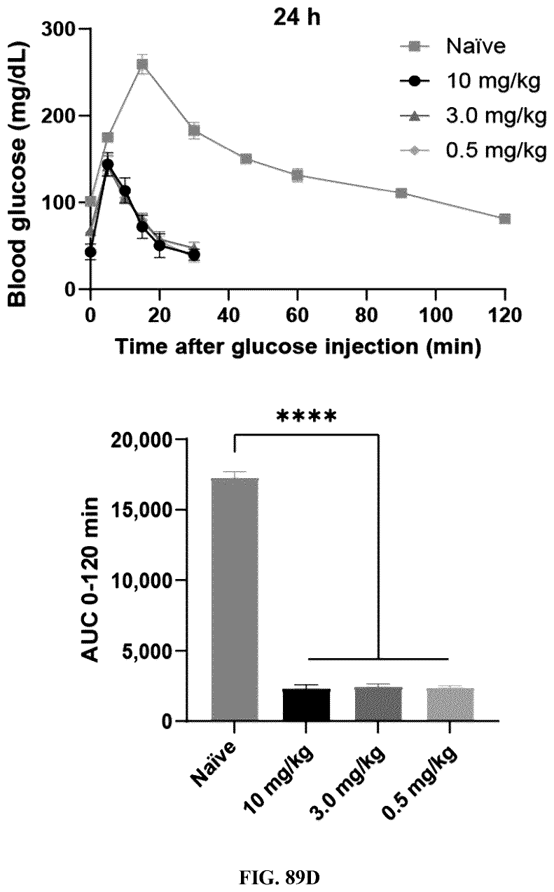

A dose response using the commercial semaglutide (0.5 mg/kg (˜5 nmol), 3 mg/kg (˜30 nmol), or 10 mg/kg (˜100 nmol), SC) allowed us to validate the WT Swiss mice model as suitable for measuring blood glucose change in addition to body weight loss (FIGS. 89A to 89D). Then, female WT mice received one single SC injection of either 10 mg/kg (˜100 nmol) of semaglutide, or 10 mg/kg (˜25 nmol) of 2a,3a (FIGS. 74A and 74B). A significant 4-5% body weight loss was observed at 24 h post injection, sustained for 10 days (2a) or 15 days (3a) (FIG. 74C).

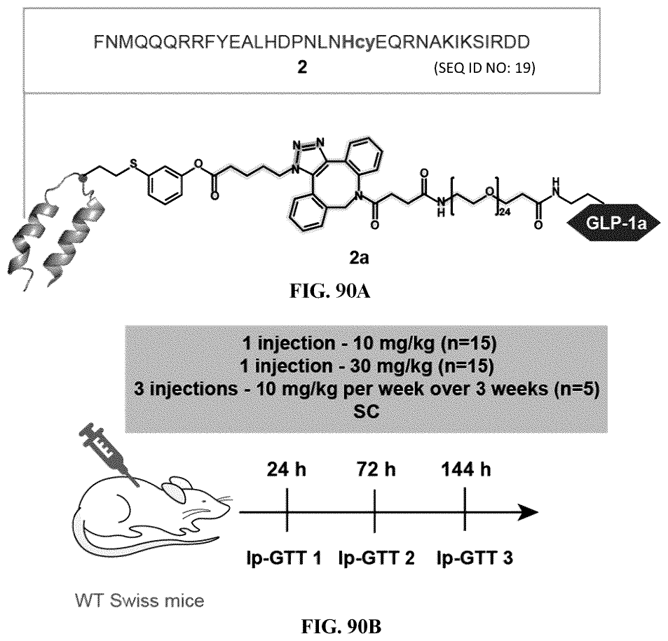

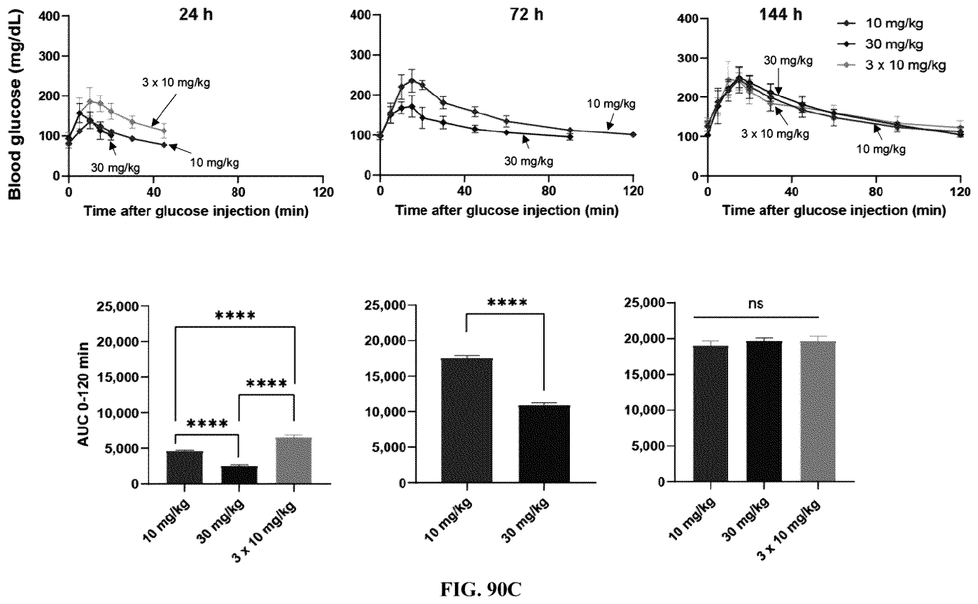

Intraperitoneal glucose tolerance tests (Ip-GTT) demonstrated blood glucose decrease for all the GLP-1 analogs compared to the naïve mice, after 24 h of injection (FIG. 74D). After 6 days, semaglutide showed no more ability to reduce blood glucose in the WT mice (AUC_{0-120 min}=21,282±795 vs 21,354+431 for the naïve) while 2a and 3a were still exerting significant activation of GLP-IR (AUC₀₋₁₂₀ₘᵢₙ=19,004±650 and 17,744±366, respectively). In addition, a stacking dose of three injections of 10 mg/kg each (˜75 nmol total), following one injection per week for three weeks induced similar efficacy as one single dose of 10 mg/kg, while a single dose of 30 mg/kg of 2a was significantly more efficient than a 10 mg/kg dose (P<0.0001), at both h and 72 h p.i, but presented no benefit at the 144-h time-point (FIGS. 90A to 90C).

In vivo IgG painting with GLP-1a sustains body weight loss and improves blood glucose management in obese Lep^{ob/ob} mice for at least 10 days after SC injection.

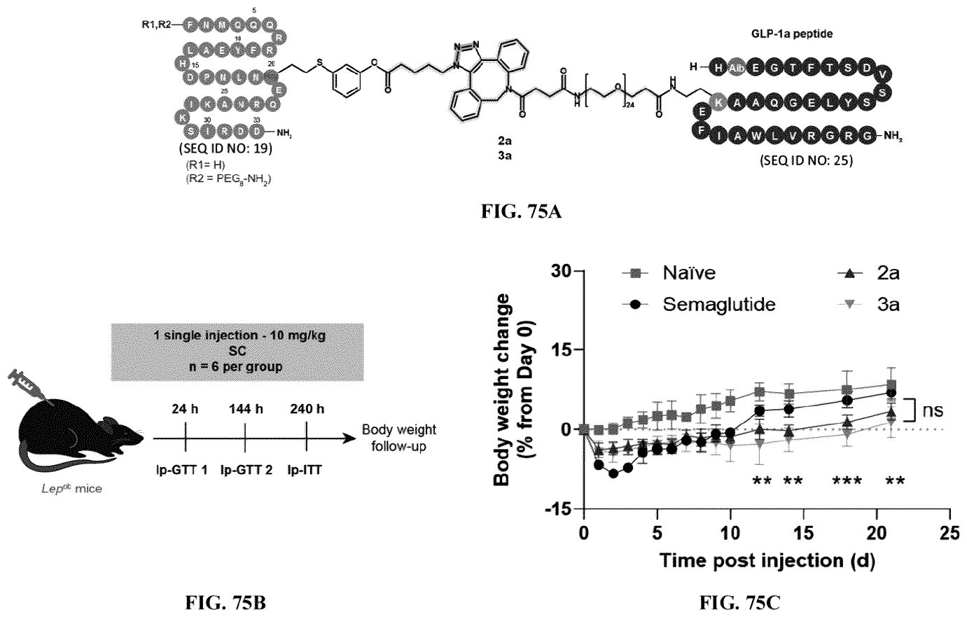

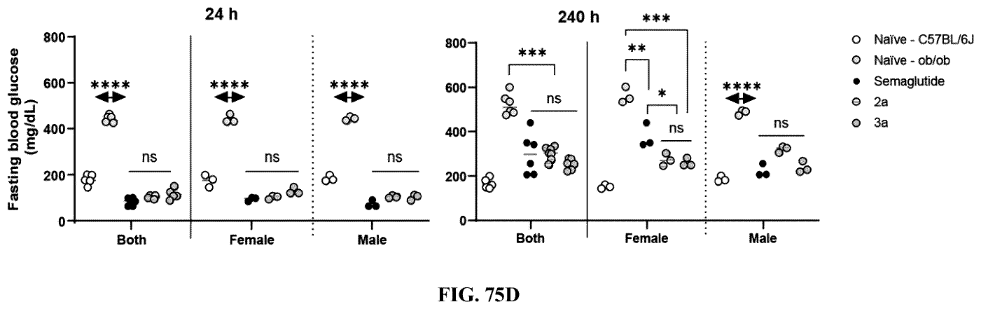

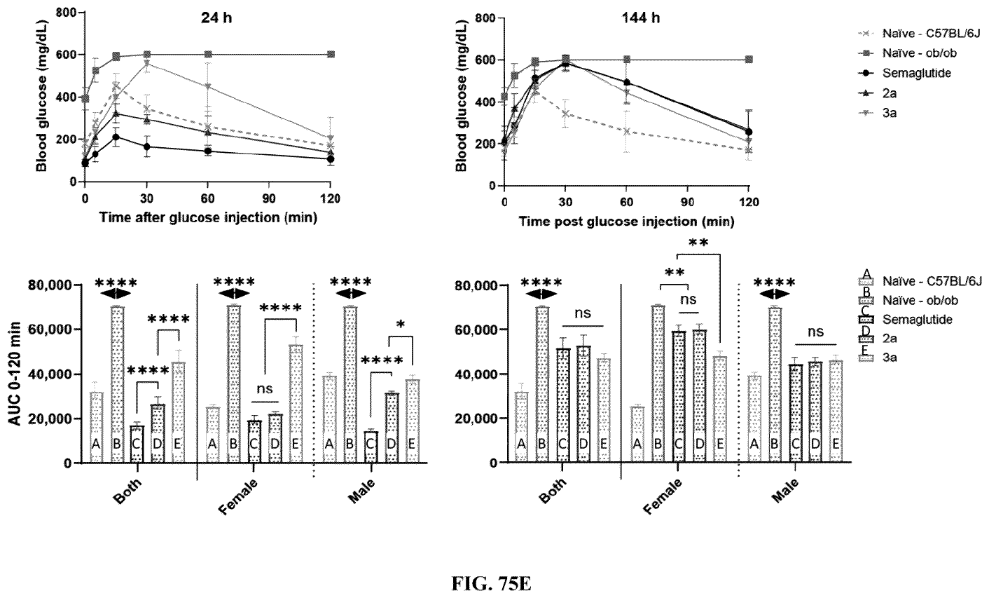

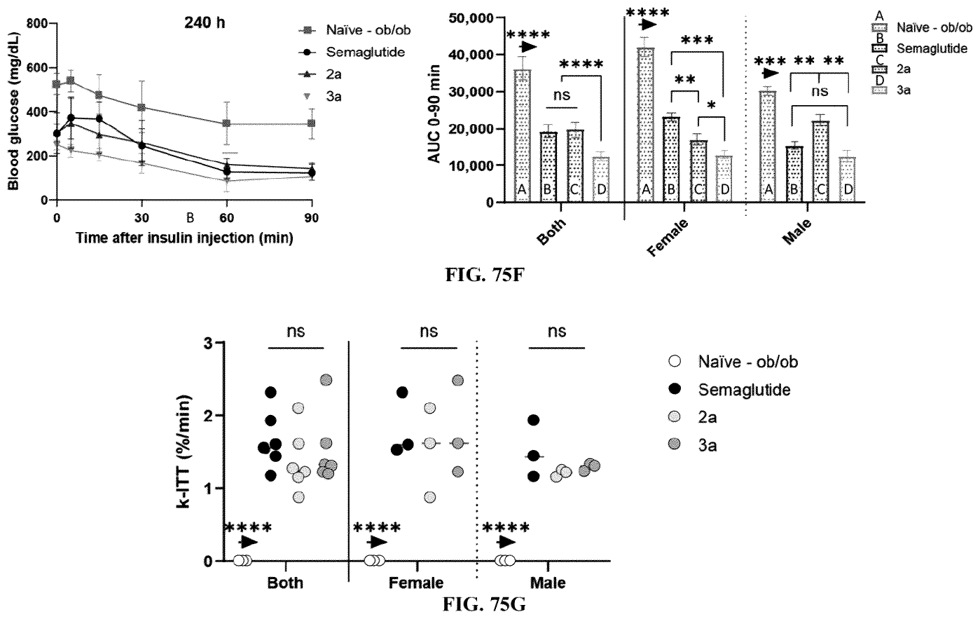

Both males and females were used for this efficacy study to avoid sex-related biases. One single injection of 10 mg/kg (˜170 nmol) of Semaglutide, or 10 mg/kg (˜45 nmol) of 2a,3a was SC in Lep^{ob/ob} mice (FIGS. 75A and 75B). Significant body weight loss (˜3.6±0.8 and 4.3±1.4%) was observed for 10 days after 2a,3a injections, respectively, with slower body weight intake up to Day 21 (FIG. 75C). Semaglutide induced about 8.3±0.5% of body weight loss for 3 days, then the intake went progressively up until 21 days p.i.

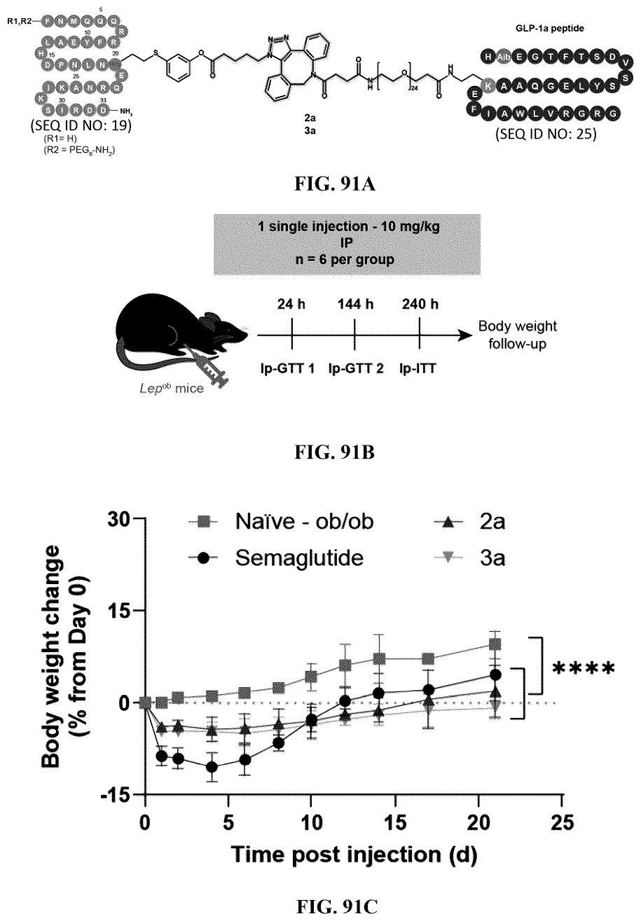

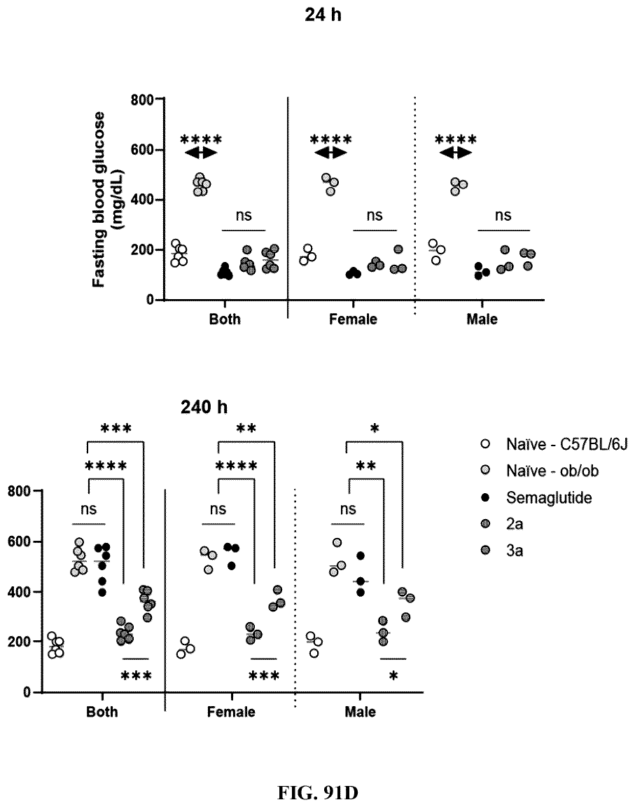

Lep^{ob/ob} are insulin resistant as confirmed by the blood glucose levels measured for the naïve, with 440±9 mg/dL vs 178±14 mg/dL for the non-obese C57BL/6J mice, both sexes combined (FIG. 91B). The cohorts were fasted for 5-6 hours then challenged with glucose injections (2 g/kg) at 24 and 72 h p.i (FIG. 91D, left), and with insulin injection (2.5 IU/kg) at day 10 p.i. Semaglutide, 2a and 3a were efficient up to 10 days p.i., enabling to down-regulate blood glucose levels significantly, in both sexes combined (FIGS. 75D and 75E). Moreover, the response to insulin observed after 10 days highlighted that 2a,3a induced similar effect than semaglutide, 3a even showing a slightly better efficacy in females (P<0.001) (FIG. 75F). The improvement of the insulin sensitivity status was confirmed using the plasma glucose disappearance rate (k-ITT) (FIG. 75G). In Lep^{ob/ob} mice, in vivo IgG painting with GLP-1 analogs demonstrated similar efficacy than semaglutide, after SC injection, even though 2a,3a contain almost 4 times less GLP-1 peptide compared to the semaglutide drug.

In Vivo IgG Painting with GLP-1a Improved Metabolic Health in Lep^{ob/ob} Mice after IP Administration as Well

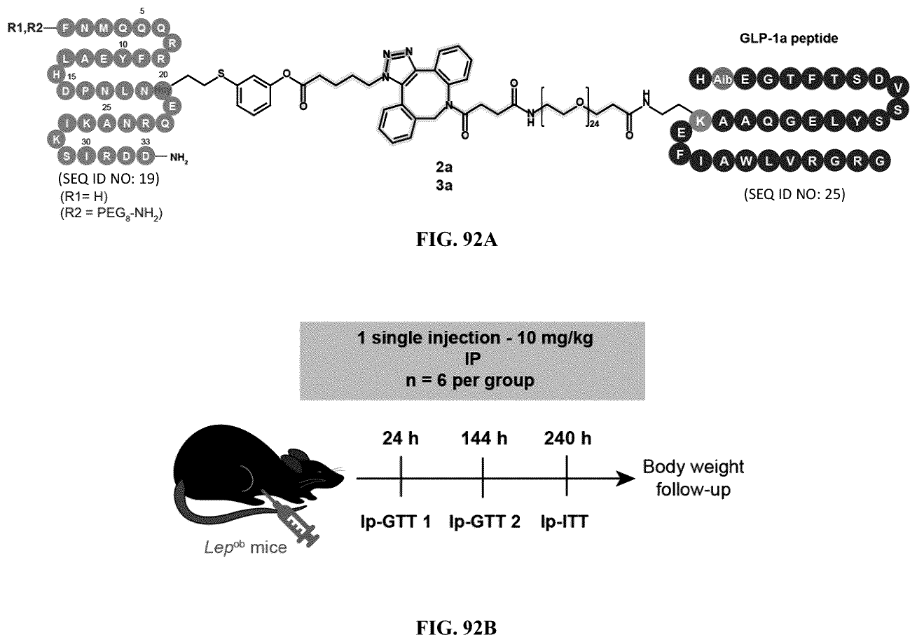

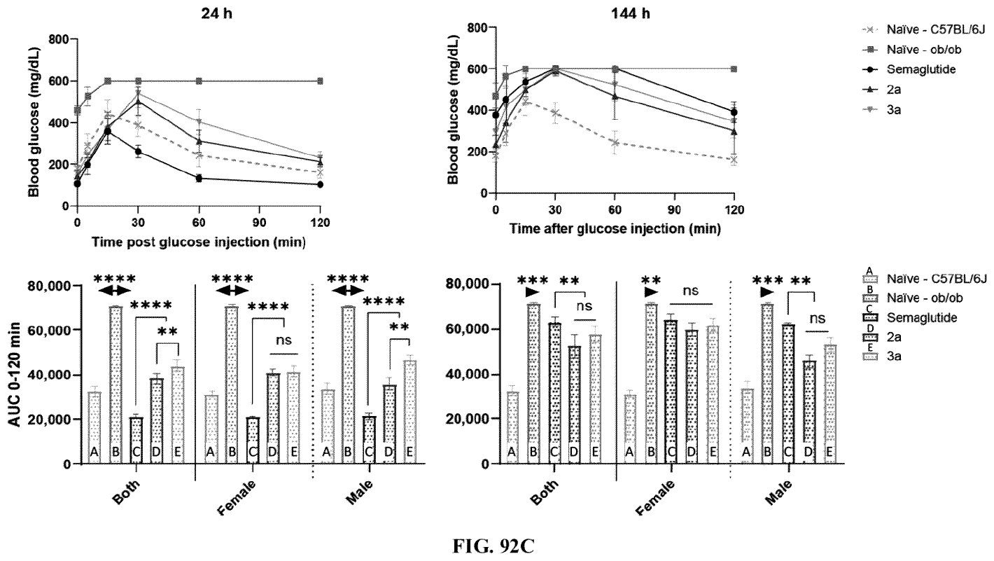

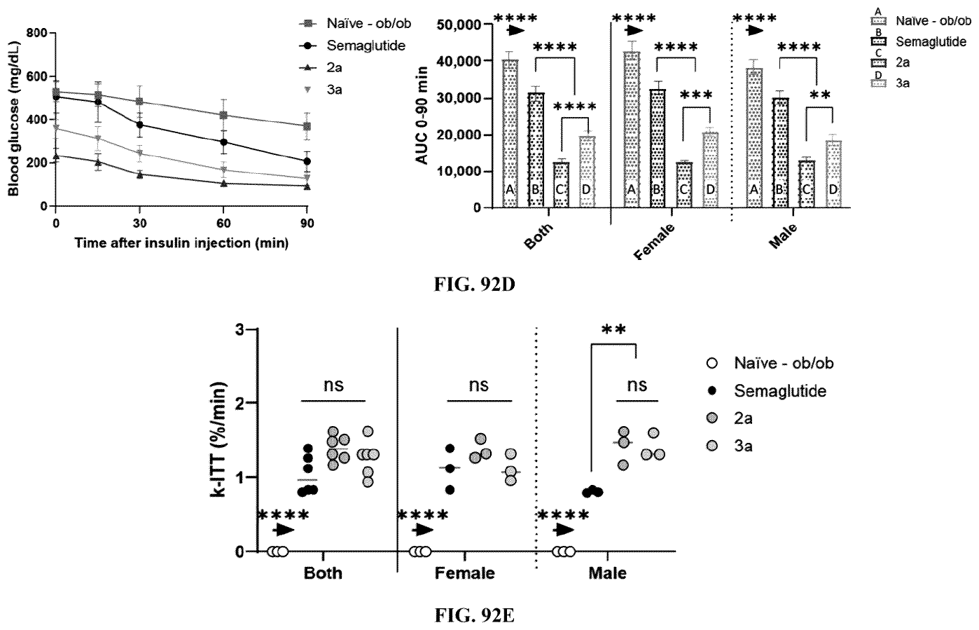

The IP route was assessed in male and female Lep^{ob/ob} mice (FIGS. 92A and 92B). Body weight loss after IP 2a,3a was comparable to the SC cohorts (P<0.0001) (FIG. 91C). However, IP semaglutide sustained body weight loss up to 7 days p.i, then followed a similar pattern as 2a,3a. Fasting blood glucose levels were maintained in the normal range for up to 6 days (FIG. 91D, right). After 10 days, semaglutide showed no more efficacy while 2a, and 3a still enabled to lower blood glucose (P<0.0001 and P<0.001, respectively). IP seems to induce a faster clearance of semaglutide while increasing the efficiency of 2a,3a (FIG. 92C). After 10 days, 2a was the most effective for reducing blood glucose in response to insulin, in both sexes, highlighting that the presence of the PEG in 3a reduced the affinity peptide efficiency to bind IgGs after IP injection (FIG. 92D). All the GLP-1 analogs enabled to reverse insulin resistance in Lep^{ob/ob} mice (P<0.0001) (FIG. 92E).

IgG Painting with Radionuclides Highlights the Payload Versatility

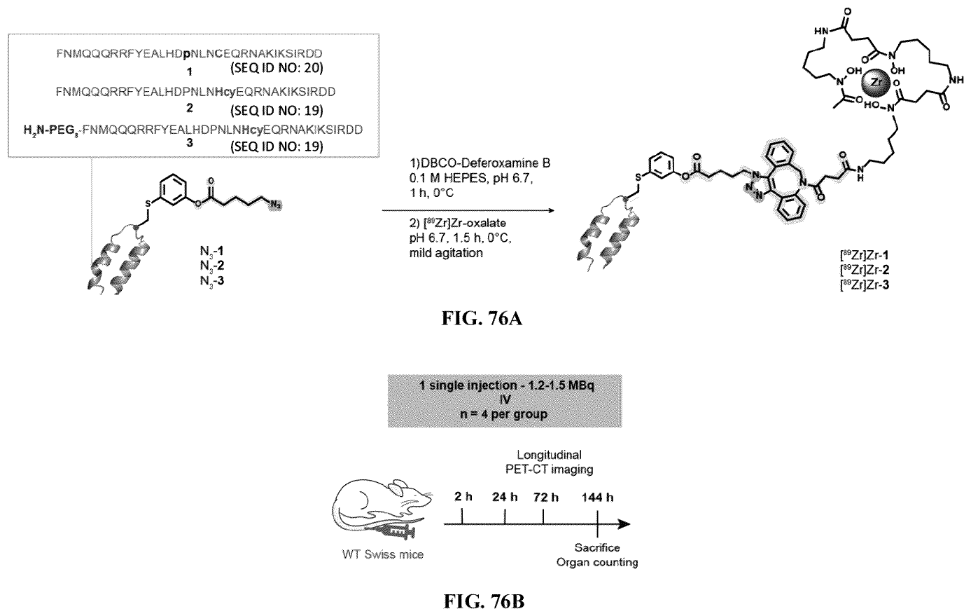

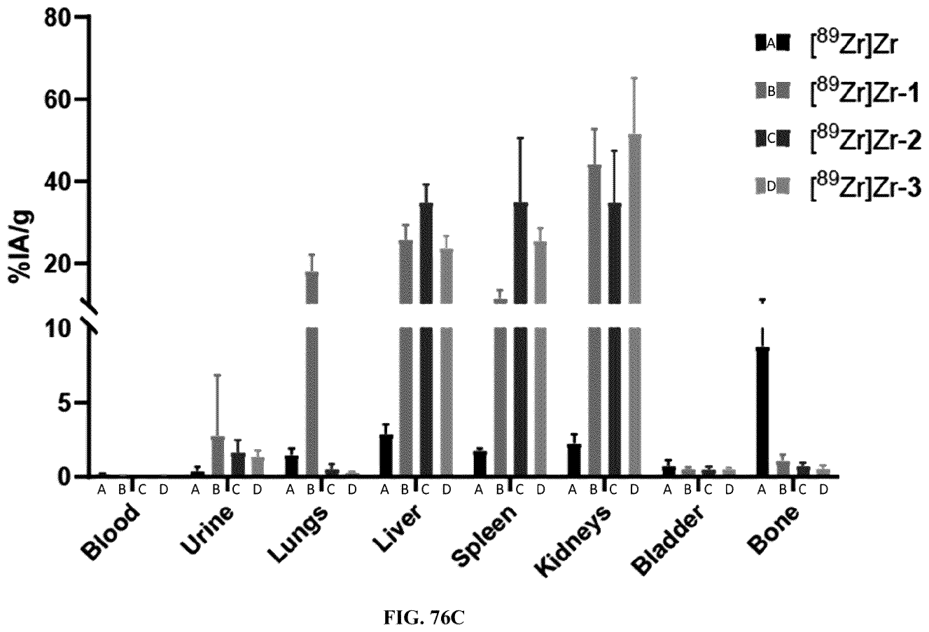

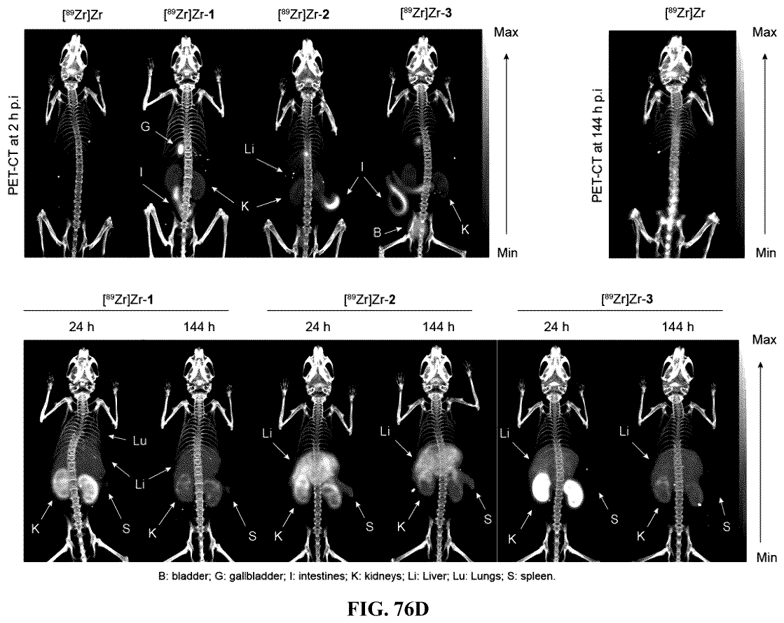

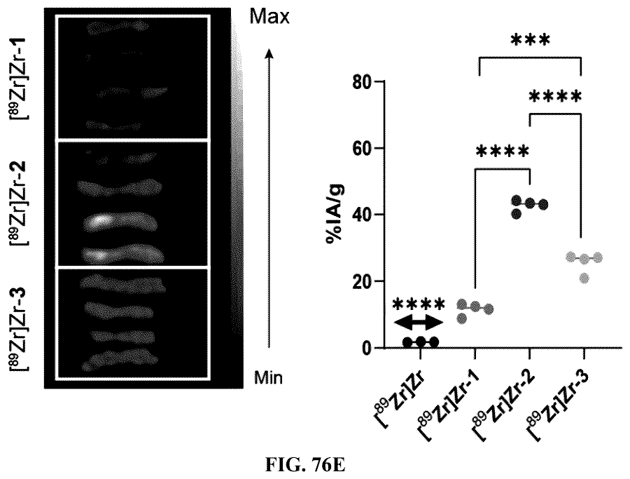

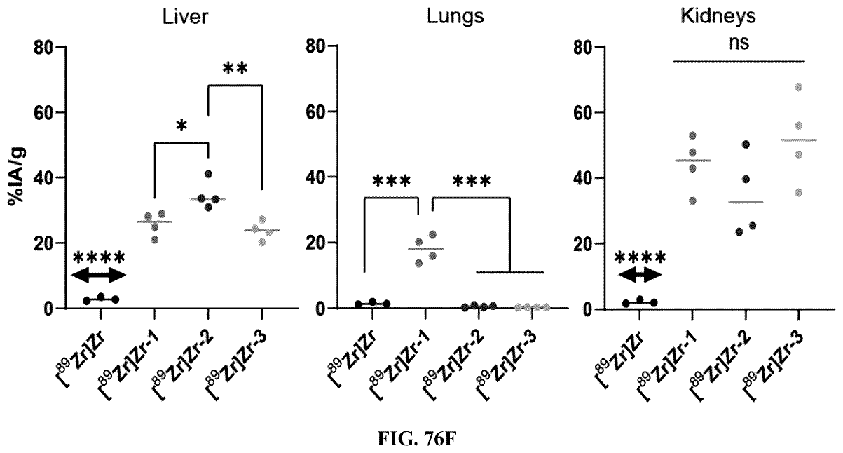

It was hypothesized that the in vivo IgG painting approach can be used as a general drug delivery platform suitable for numerous kinds of payloads. Radionuclides are among the most challenging payloads but are particularly useful for in vivo detection and quantification. N₃-1-3-I was radiolabeled using zirconium-89 and the fate of [⁸⁹Zr]Zr-1-3 was assessed in female WT mice, after either intravenous (IV), SC, or IP injection of 1.2-1.5 MBq (FIGS. 76A to 76B and FIGS. 93A to 95H). In mice, the highest amounts of FcRn receptors were found in the following tissues: small intestine, spleen, large intestine, kidney, liver and lungs (32, 33). As functional FcRn binds, transports, and recycles IgGs, after the IV injection of [⁸⁹Zr]Zr-1-3, the resulting [⁸⁹Zr]Zr-IgGs were most likely expected to be located in those very same tissues.

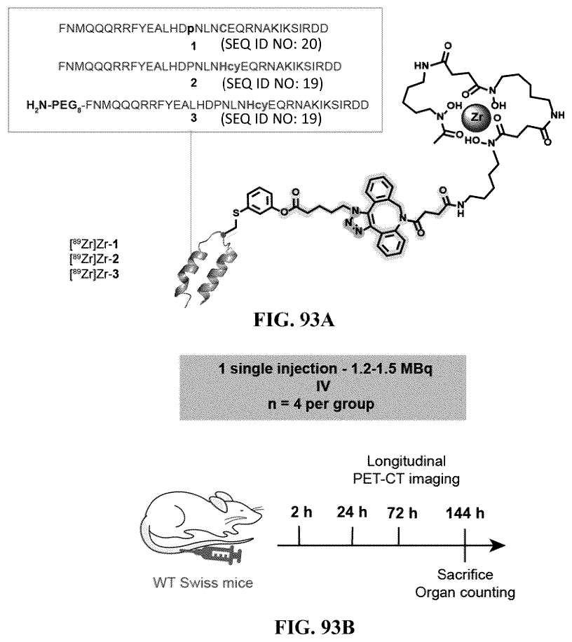

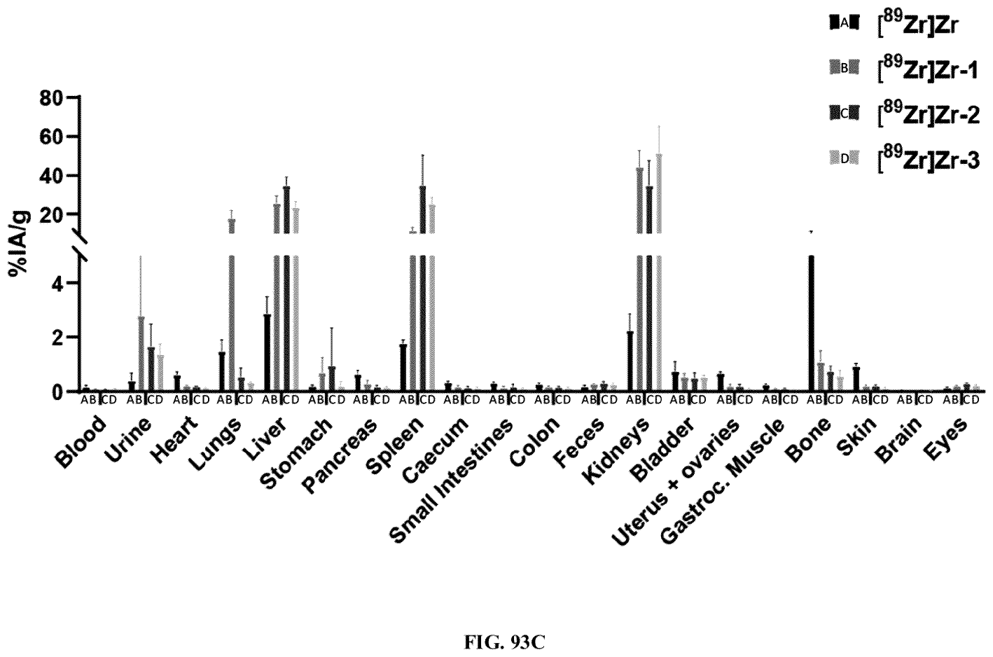

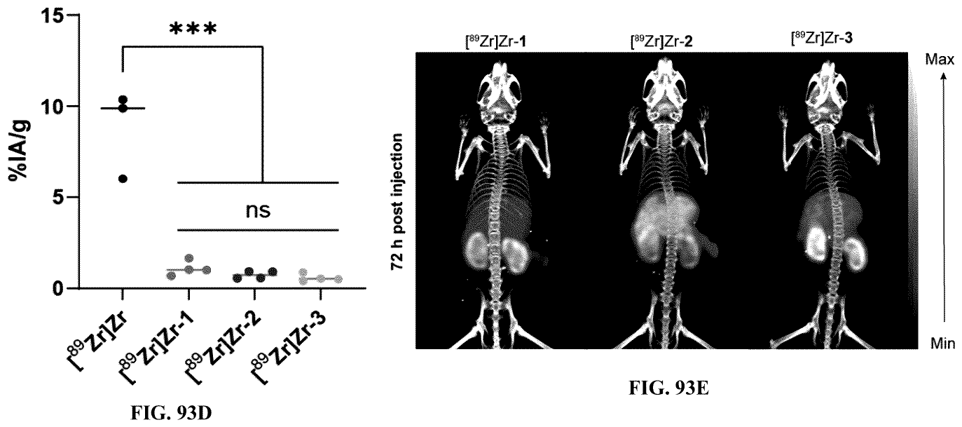

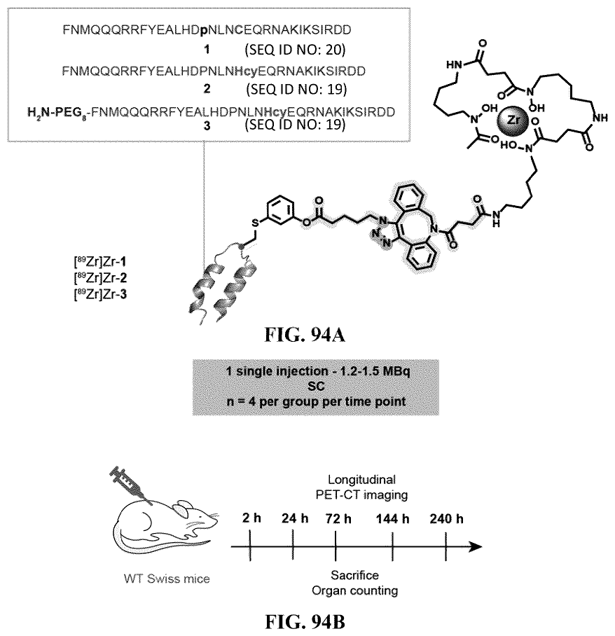

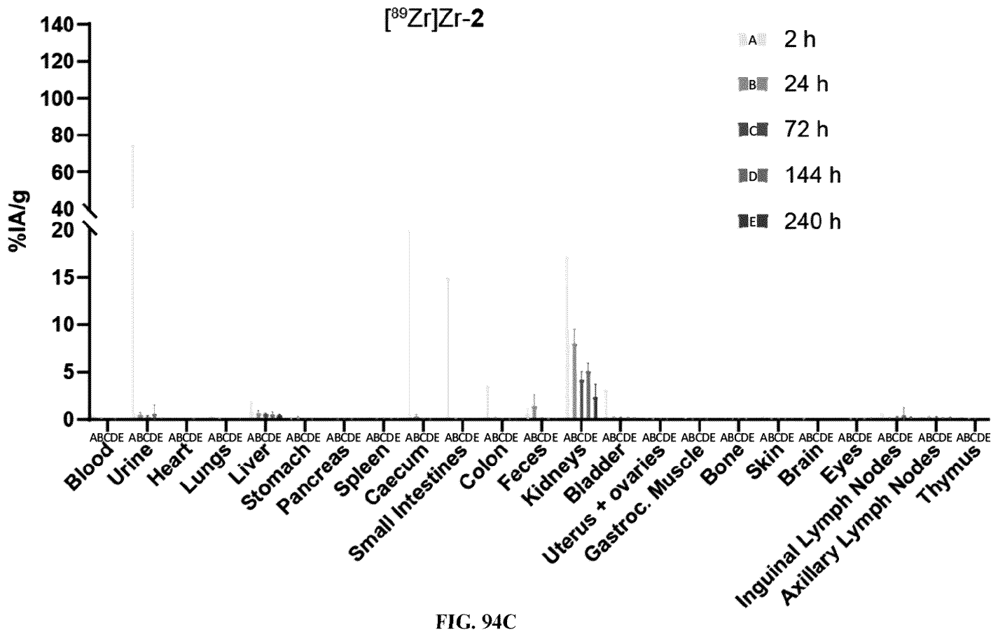

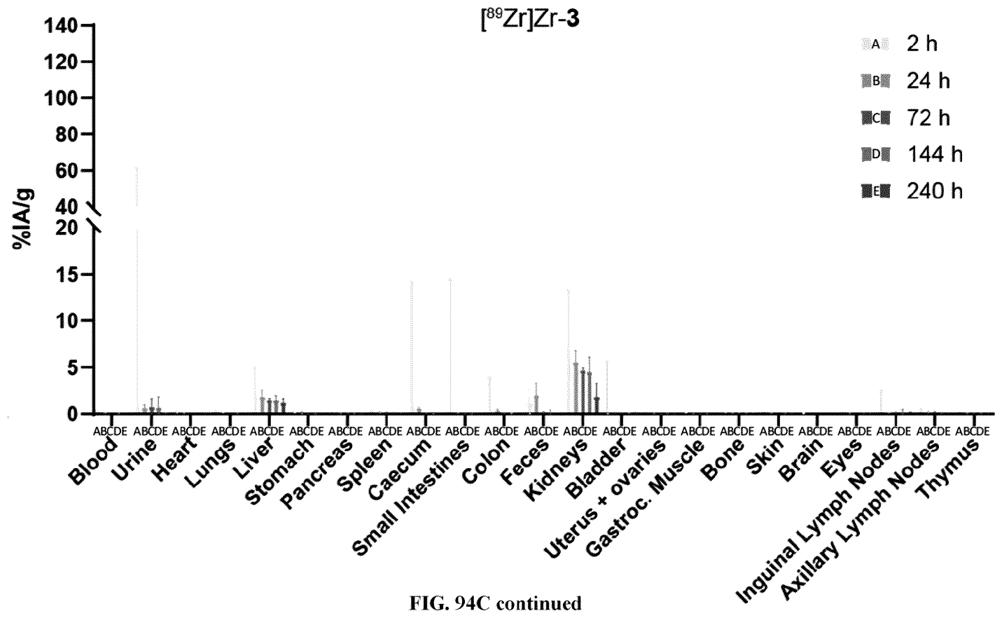

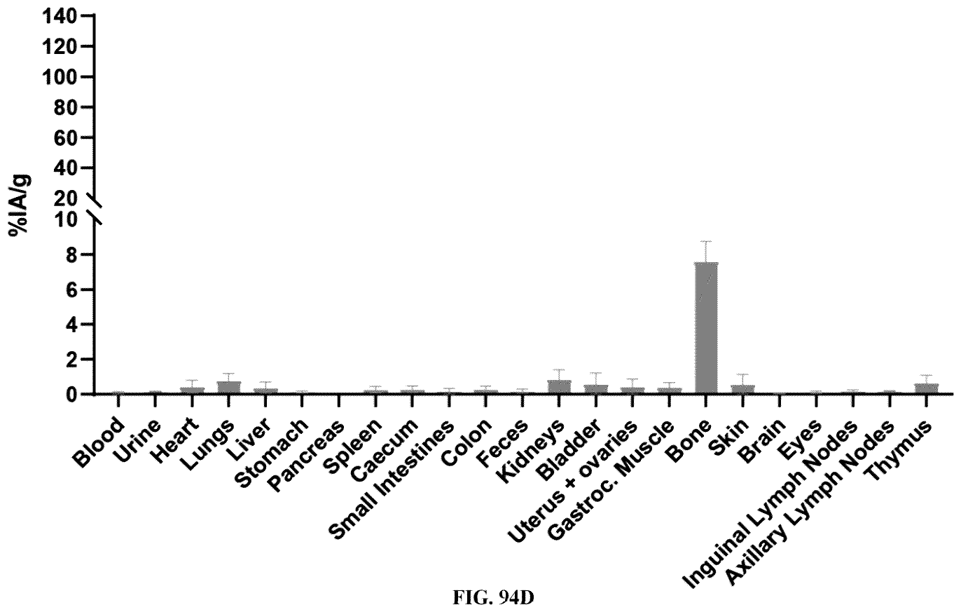

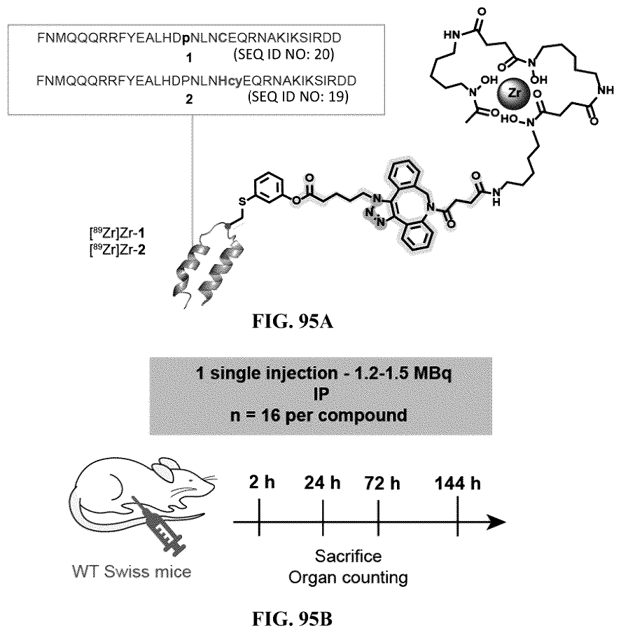

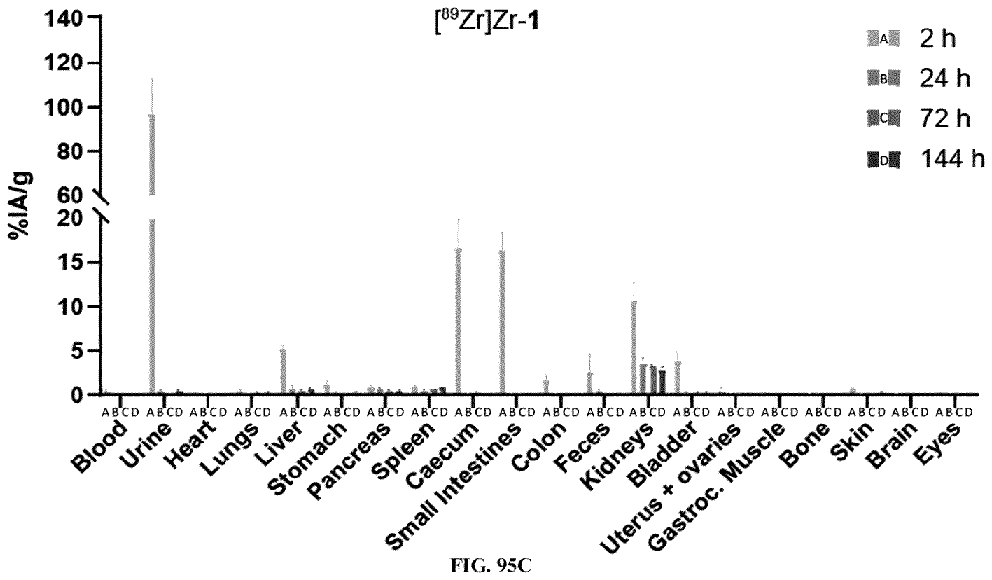

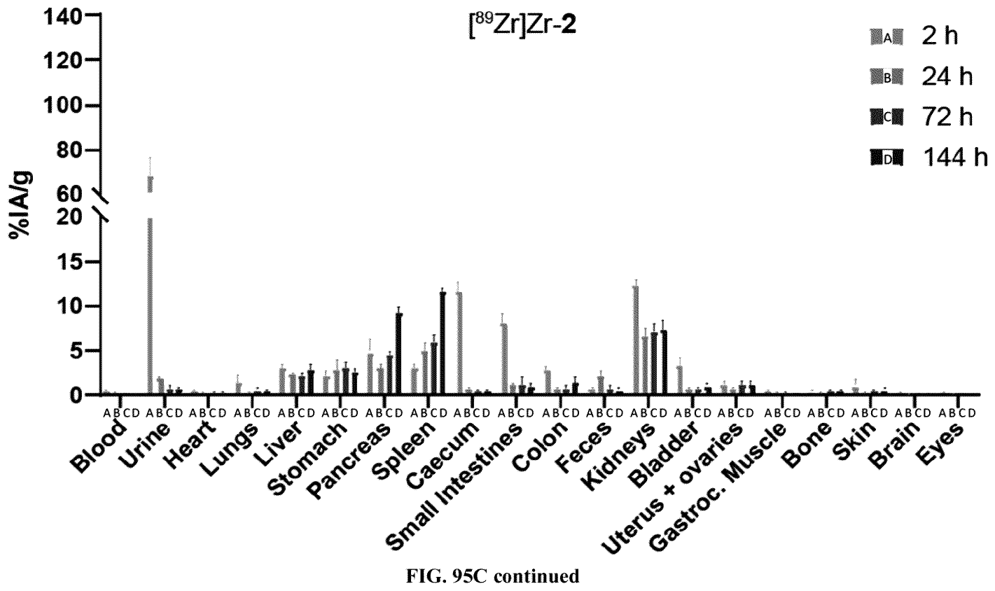

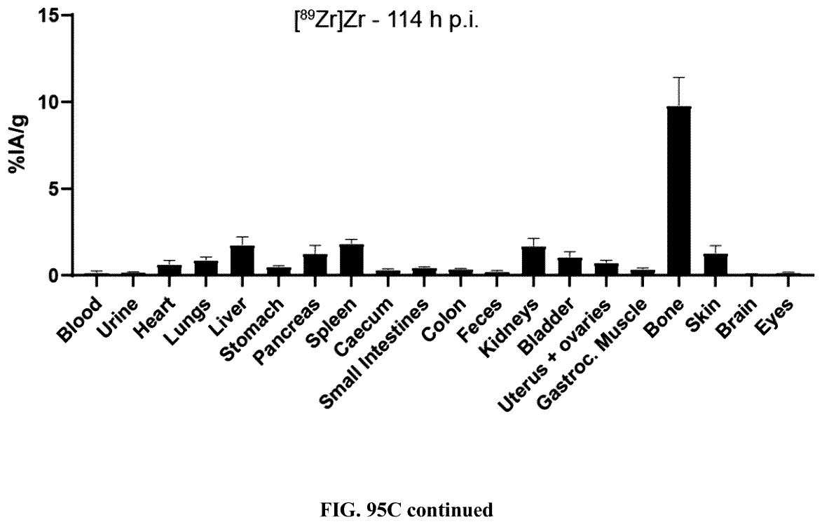

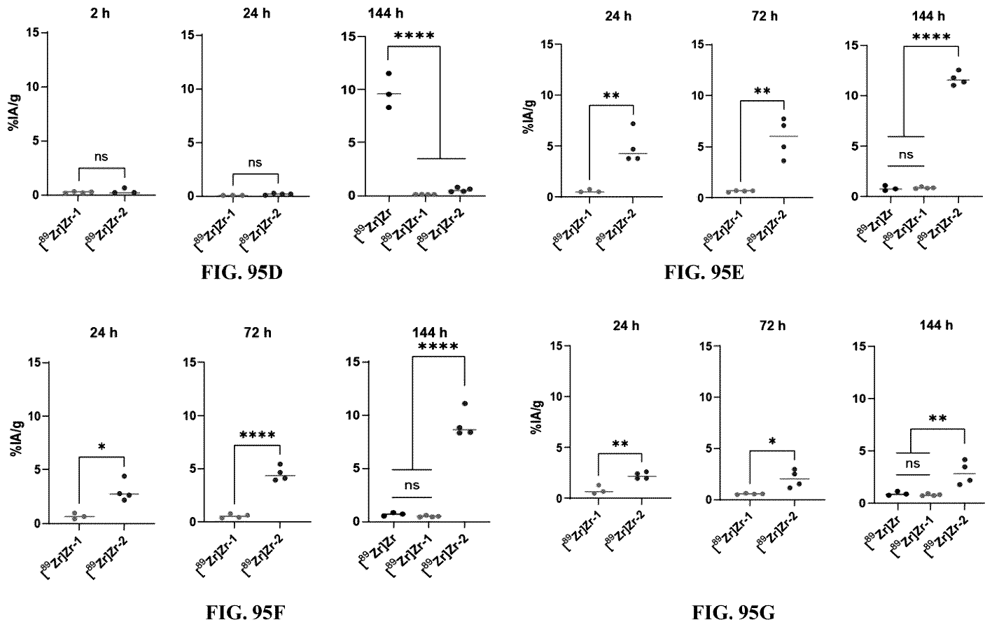

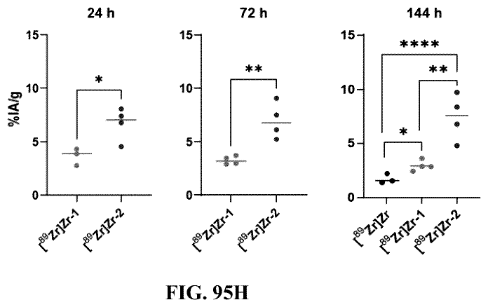

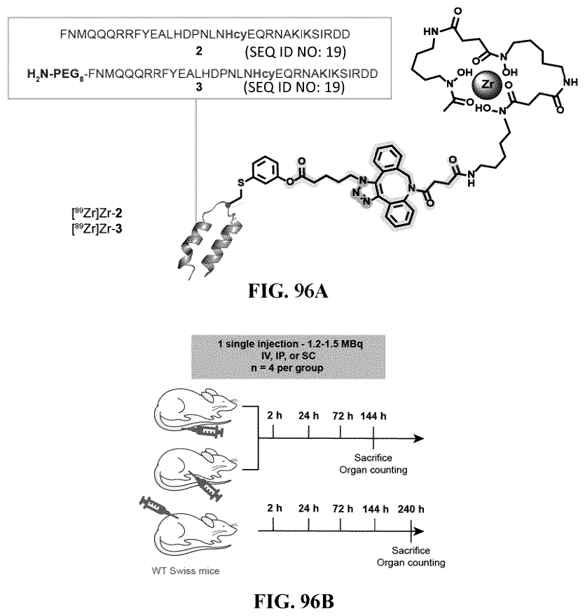

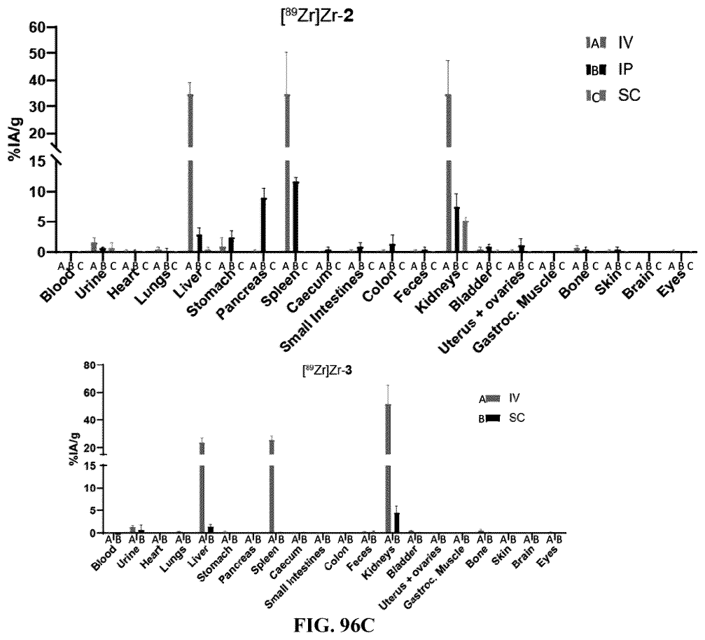

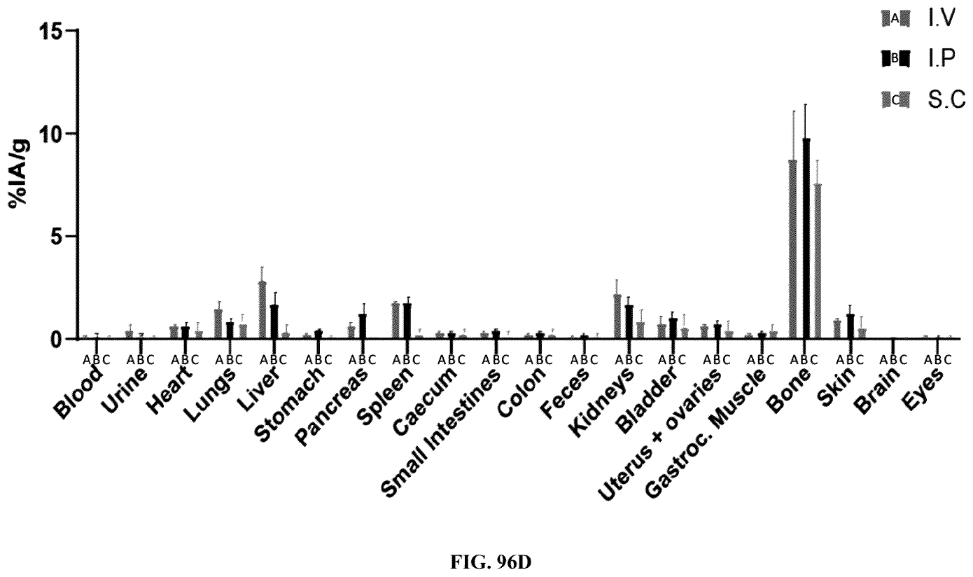

Longitudinal PET-CT imaging and ex vivo quantifications in organs confirmed the uptake of IV injected [⁸⁹Zr]Zr-1-3 in organs rich in FcRn, for up to 6 days p.i. (FIGS. 76A to 76F, FIGS. 93A to 93E, and Table 22). De-chelation was neglectable over the 6-days experiment period, confirming that the uptake quantified in the tissues is specific (FIG. 93D (34). The SC route appeared to be less efficient than the IV, as the uptake measured in tissues is neglectable after 24 h, except for the kidneys and the liver (FIGS. 94A to 94D and Tables 23 and 24). Longitudinal PET-CT also indicated that most of the signal was stuck at the injection point for 10 days, suggesting that SC is less suitable for radionuclide transfer using the in vivo IgG painting approach than IV injection. IP injection showed better suitability than SC, with a persistent uptake organs rich in FcRn after injection of [⁸⁹Zr]Zr-2 (FIGS. 95A to 96D and Table 25).

Altogether, these results indicate that in vivo IgG painting using electrophile peptide 2,3-I may be impactful for the development of long-acting drugs by proposing a broad platform freed from any cumbersome in vitro antibody engineering.

TABLE 22
Biodistribution of the affinity peptides [⁸⁹Zr]Zr-
1-3 in the main organs at 144 h after intravenous injection
in female WT Swiss mice. Quantification was performed
using Wizard² 3″ (Perkin Elmer) gamma counter. Data
are represented as mean ± SEM (n = 3-4 mice per group).

| Mean ± SEM % IA/g | [⁸⁹Zr]Zr-1 | [⁸⁹Zr]Zr-2 | [⁸⁹Zr]Zr-3 |
|---|---|---|---|
| Blood | 0.09 ± 0.01 | 0.09 ± 0.00 | 0.07 ± 0.04 |
| Urine | 2.79 ± 3.11 | 1.67 ± 0.61 | 1.38 ± 0.27 |
| Heart | 0.21 ± 0.01 | 0.18 ± 0.01 | 0.15 ± 0.01 |
| Lungs | 18.0 ± 3.25 | 0.56 ± 0.25 | 0.34 ± 0.03 |
| Liver | 25.7 ± 2.79 | 34.7 ± 3.18 | 23.8 ± 2.03 |
| Stomach | 0.70 ± 0.45 | 0.95 ± 1.05 | 0.20 ± 0.14 |
| Pancreas | 0.28 ± 0.10 | 0.18 ± 0.03 | 0.16 ± 0.02 |
| Spleen | 11.5 ± 1.34 | 42.8 ± 1.26 | 25.5 ± 2.29 |
| Caecum | 0.18 ± 0.04 | 0.14 ± 0.03 | 0.13 ± 0.03 |
| Small intestines | 0.14 ± 0.04 | 0.17 ± 0.08 | 0.11 ± 0.02 |
| Colon | 0.18 ± 0.02 | 0.16 ± 0.02 | 0.13 ± 0.02 |
| Feces | 0.27 ± 0.02 | 0.30 ± 0.06 | 0.26 ± 0.06 |
| Kidneys | 44.2 ± 6.24 | 34.7 ± 10.2 | 51.6 ± 10.3 |
| Bladder | 0.54 ± 0.08 | 0.50 ± 0.14 | 0.55 ± 0.05 |
| Uterus | 0.19 ± 0.07 | 0.20 ± 0.07 | 0.10 ± 0.03 |
| Gastroc. Muscle | 0.10 ± 0.01 | 0.10 ± 0.01 | 0.07 ± 0.01 |
| Bone | 1.09 ± 0.28 | 0.74 ± 0.18 | 0.58 ± 0.15 |
| Skin | 0.20 ± 0.03 | 0.21 ± 0.02 | 0.12 ± 0.02 |
| Brain | 0.03 ± 0.00 | 0.03 ± 0.01 | 0.03 ± 0.00 |
| Eyes | 0.20 ± 0.01 | 0.29 ± 0.03 | 0.23 ± 0.03 |

% IA/g: percent of injected activity per gram; Gastroc. muscle: gastrocnemius muscle.

TABLE 23
Biodistribution of the affinity peptides [⁸⁹Zr]Zr-2 in the main organs at 2
h, 24 h, 72 h, 144 h, and 240 h after subcutaneous injection in female WT Swiss
mice. Quantification was performed using Wizard² 3″ (Perkin Elmer) gamma counter.
Data are represented as mean ± SEM (n = 3-4 mice per group for each time point).

| Mean ± SEM | [⁸⁹Zr]Zr-2 | | | | |
|---|---|---|---|---|---|
| % IA/g | 2 h | 24 h | 72 h | 144 h | 240 h |
| Blood | 0.05 ± 0.07 | 0.07 ± 0.04 | 0.04 ± 0.01 | 0.03 ± 0.01 | 0.02 ± 0.00 |
| Urine | 67.0 ± 35.6 | 0.66 ± 0.29 | 0.54 ± 0.29 | 0.45 ± 0.41 | 0.08 ± 0.02 |
| Heart | 0.35 ± 0.07 | 0.06 ± 0.00 | 0.06 ± 0.02 | 0.04 ± 0.01 | 0.04 ± 0.00 |
| Lungs | 0.80 ± 0.36 | 0.12 ± 0.04 | 0.11 ± 0.05 | 0.09 ± 0.04 | 0.05 ± 0.01 |
| Liver | 2.45 ± 0.42 | 1.22 ± 0.65 | 1.34 ± 0.74 | 1.08 ± 0.73 | 0.49 ± 0.05 |
| Stomach | 0.19 ± 0.09 | 0.14 ± 0.10 | 0.05 ± 0.03 | 0.07 ± 0.06 | 0.01 ± 0.00 |
| Pancreas | 0.33 ± 0.13 | 0.08 ± 0.06 | 0.10 ± 0.06 | 0.07 ± 0.06 | 0.03 ± 0.01 |
| Spleen | 0.39 ± 0.15 | 0.12 ± 0.03 | 0.13 ± 0.04 | 0.12 ± 0.05 | 0.07 ± 0.01 |
| Caecum | 10.7 ± 10.5 | 0.39 ± 0.13 | 0.09 ± 0.04 | 0.06 ± 0.04 | 0.02 ± 0.00 |
| Small | 9.21 ± 5.60 | 0.07 ± 0.02 | 0.04 ± 0.01 | 0.03 ± 0.03 | 0.01 ± 0.00 |
| intestines | | | | | |
| Colon | 2.44 ± 2.09 | 0.21 ± 0.06 | 0.08 ± 0.04 | 0.07 ± 0.05 | 0.02 ± 0.00 |
| Feces | 0.57 ± 0.45 | 1.58 ± 0.80 | 0.19 ± 0.07 | 0.19 ± 0.14 | 0.05 ± 0.01 |
| Kidneys | 32.1 ± 13.3 | 17.3 ± 9.68 | 16.1 ± 11.5 | 15.2 ± 10.7 | 2.41 ± 0.95 |
| Bladder | 3.29 ± 1.97 | 0.22 ± 0.03 | 0.20 ± 0.04 | 0.18 ± 0.06 | 0.12 ± 0.02 |
| Uterus | 0.27 ± 0.06 | 0.04 ± 0.01 | 0.06 ± 0.04 | 0.04 ± 0.02 | 0.04 ± 0.01 |
| Gastroc. | 0.16 ± 0.03 | 0.03 ± 0.00 | 0.03 ± 0.01 | 0.03 ± 0.01 | 0.02 ± 0.00 |
| Muscle | | | | | |
| Bone | 0.20 ± 0.03 | 0.06 ± 0.02 | 0.17 ± 0.10 | 0.10 ± 0.05 | 0.06 ± 0.00 |
| Skin | 0.51 ± 0.12 | 0.07 ± 0.02 | 0.10 ± 0.02 | 0.06 ± 0.03 | 0.05 ± 0.00 |
| Brain | 0.05 ± 0.01 | 0.01 ± 0.00 | 0.01 ± 0.00 | 0.01 ± 0.00 | 0.01 ± 0.00 |
| Eyes | 0.26 ± 0.04 | 0.05 ± 0.01 | 0.05 ± 0.00 | 0.05 ± 0.00 | 0.08 ± 0.02 |
| Inguinal | 1.49 ± 0.49 | 0.49 ± 0.55 | 0..36 ± 0.23 | 0.23 ± 0.16 | 0.15 ± 0.06 |
| lymph nodes | | | | | |
| Axillary | 0.53 ± 0.06 | 0.23 ± 0.05 | 0.20 ± 0.02 | 0.13 ± 0.02 | 0.14 ± 0.05 |
| lymph nodes | | | | | |
| Thymus | 0.42 ± 0.13 | 0.08 ± 0.01 | 0.09 ± 0.04 | 0.07 ± 0.02 | 0.08 ± 0.03 |

% IA/g: percent of injected activity per gram; Gastroc. muscle: gastrocnemius muscle.

TABLE 24
Biodistribution of the affinity peptides [⁸⁹Zr]Zr-3 in the main organs at 2
h, 24 h, 72 h, 144 h, and 240 h after subcutaneous injection in female WT Swiss
mice. Quantification was performed using Wizard² 3″ (Perkin Elmer) gamma counter.
Data are represented as mean ± SEM (n = 3-4 mice per group for each time point).

| Mean ± SEM | [⁸⁹Zr]Zr-3 | | | | |
|---|---|---|---|---|---|
| % IA/g | 2 h | 24 h | 72 h | 144 h | 240 h |
| Blood | 0.49 ± 0.01 | 0.11 ± 0.03 | 0.04 ± 0.01 | 0.03 ± 0.01 | 0.01 ± 0.00 |
| Urine | 62.5 ± 21.3 | 0.65 ± 0.22 | 0.78 ± 0.66 | 0.71 ± 0.85 | 0.08 ± 0.02 |
| Heart | 0.48 ± 0.29 | 0.06 ± 0.01 | 0.04 ± 0.00 | 0.04 ± 0.00 | 0.03 ± 0.01 |
| Lungs | 0.47 ± 0.03 | 0.10 ± 0.01 | 0.06 ± 0.01 | 0.05 ± 0.00 | 0.04 ± 0.01 |
| Liver | 5.30 ± 0.22 | 1.87 ± 0.50 | 1.53 ± 0.11 | 1.50 ± 0.35 | 1.23 ± 0.34 |
| Stomach | 0.42 ± 0.20 | 0.13 ± 0.09 | 0.03 ± 0.01 | 0.03 ± 0.01 | 0.01 ± 0.01 |
| Pancreas | 0.33 ± 0.11 | 0.05 ± 0.01 | 0.04 ± 0.01 | 0.04 ± 0.02 | 0.03 ± 0.00 |
| Spleen | 0.46 ± 0.06 | 0.18 ± 0.05 | 0.16 ± 0.01 | 0.14 ± 0.01 | 0.10 ± 0.01 |
| Caecum | 14.4 ± 6.85 | 0.62 ± 0.09 | 0.05 ± 0.01 | 0.08 ± 0.05 | 0.01 ± 0.00 |
| Small | 14.8 ± 1.69 | 0.13 ± 0.03 | 0.03 ± 0.00 | 0.03 ± 0.00 | 0.01 ± 0.00 |
| intestines | | | | | |
| Colon | 4.10 ± 1.78 | 0.38 ± 0.09 | 0.05 ± 0.02 | 0.05 ± 0.04 | 0.02 ± 0.00 |
| Feces | 1.12 ± 1.10 | 2.05 ± 0.90 | 0.17 ± 0.05 | 0.20 ± 0.16 | 0.03 ± 0.01 |
| Kidneys | 13.5 ± 1.31 | 5.58 ± 0.83 | 4.69 ± 0.17 | 4.53 ± 1.32 | 1.86 ± 1.21 |
| Bladder | 5.89 ± 5.40 | 0.17 ± 0.02 | 0.11 ± 0.01 | 0.10 ± 0.02 | 0.11 ± 0.03 |
| Uterus | 0.26 ± 0.07 | 0.05 ± 0.01 | 0.03 ± 0.01 | 0.04 ± 0.01 | 0.03 ± 0.01 |
| Gastroc. | 0.20 ± 0.07 | 0.03 ± 0.00 | 0.02 ± 0.00 | 0.02 ± 0.00 | 0.02 ± 0.00 |
| Muscle | | | | | |
| Bone | 0.26 ± 0.08 | 0.07 ± 0.01 | 0.09 ± 0.01 | 0.07 ± 0.01 | 0.08 ± 0.01 |
| Skin | 0.36 ± 0.04 | 0.07 ± 0.01 | 0.06 ± 0.00 | 0.05 ± 0.01 | 0.05 ± 0.01 |
| Brain | 0.07 ± 0.03 | 0.01 ± 0.00 | 0.01 ± 0.00 | 0.01 ± 0.00 | 0.01 ± 0.00 |
| Eyes | 0.25 ± 0.14 | 0.05 ± 0.01 | 0.03 ± 0.00 | 0.05 ± 0.01 | 0.05 ± 0.01 |
| Inguinal | 2.70 ± 1.83 | 0.12 ± 0.02 | 0.16 ± 0.04 | 0.22 ± 0.19 | 0.14 ± 0.06 |
| lymph nodes | | | | | |
| Axillary | 0.69 ± 0.27 | 0.18 ± 0.08 | 0.18 ± 0.03 | 0.13 ± 0.03 | 0.12 ± 0.02 |
| lymph nodes | | | | | |
| Thymus | 0.28 ± 0.03 | 0.07 ± 0.01 | 0.05 ± 0.01 | 0.05 ± 0.01 | 0.05 ± 0.01 |

% IA/g: percent of injected activity per gram; Gastroc. muscle: gastrocnemius muscle.

TABLE 25
Biodistribution of the affinity peptides [⁸⁹Zr]Zr-1, 2 in the main
organs at 2 h, 24 h, 72 h, and 144 h after intraperitoneal injection in female
WT Swiss mice. Quantification was performed using Wizard² 3″
(Perkin Elmer) gamma counter. Data are represented as mean ±
SEM (n = 3-4 mice per group for each time point).

| Mean ± | | | | | | | | |
|---|---|---|---|---|---|---|---|---|
| SEM | [⁸⁹Zr]Zr-1 | | | | [⁸⁹Zr]Zr-2 | | | |
| % IA/g | 2 h | 24 h | 72 h | 144 h | 2 h | 24 h | 72 h | 144 h |
| Blood | 0.52 ± | 0.10 ± | 0.03 ± | 0.04 ± | 0.34 ± | 0.28 ± | 0.16 ± | 0.10 ± |
| | 0.09 | 0.01 | 0.00 | 0.01 | 0.19 | 0.06 | 0.01 | 0.01 |
| Urine | 93.9 ± | 0.38 ± | 0.14 ± | 0.40 ± | 68.2 ± | 1.84 ± | 0.71 ± | 0.72 ± |
| | 23.3 | 0.18 | 0.04 | 0.24 | 9.98 | 0.39 | 0.50 | 0.11 |
| Heart | 0.36 ± | 0.09 ± | 0.08 ± | 0.08 ± | 0.39 ± | 0.18 ± | 0.20 ± | 0.22 ± |
| | 0.07 | 0.04 | 0.04 | 0.01 | 0.06 | 0.05 | 0.04 | 0.05 |
| Lungs | 0.59 ± | 0.12 ± | 0.32 ± | 0.27 ± | 1.46 ± | 0.21 ± | 0.55 ± | 0.37 ± |
| | 0.09 | 0.03 | 0.25 | 0.17 | 1.07 | 0.09 | 0.29 | 0.11 |
| Liver | 5.21 ± | 0.82 ± | 0.56 ± | 0.79 ± | 3.12 ± | 2.25 ± | 2.04 ± | 2.89 ± |
| | 0.49 | 0.32 | 0.04 | 0.07 | 0.42 | 0.27 | 0.70 | 0.92 |
| Stomach | 1.31 ± | 0.30 ± | 0.15 ± | 0.29 ± | 2.06 ± | 3.71 ± | 3.03 ± | 2.53 ± |
| | 0.58 | 0.08 | 0.02 | 0.09 | 0.82 | 0.99 | 0.97 | 0.76 |
| Pancreas | 1.01 ± | 0.68 ± | 0.53 ± | 0.48 ± | 4.81 ± | 3.00 ± | 4.51 ± | 9.13 ± |
| | 0.23 | 0.20 | 0.16 | 0.05 | 1.76 | 0.71 | 0.51 | 0.97 |
| Spleen | 0.95 ± | 0.61 ± | 0.64 ± | 0.86 ± | 3.15 ± | 4.87 ± | 5.85 ± | 11.7 ± |
| | 0.32 | 0.11 | 0.05 | 0.06 | 0.39 | 1.16 | 1.55 | 0.49 |
| Caecum | 16.51 ± | 0.21 ± | 0.17 ± | 0.11 ± | 11.7 ± | 0.71 ± | 0.41 ± | 0.52 ± |
| | 5.42 | 0.04 | 0.13 | 0.03 | 1.28 | 0.22 | 0.14 | 0.16 |
| Small | 16.37 ± | 0.17 ± | 0.10 ± | 0.18 ± | 8.04 ± | 1.11 ± | 1.25 ± | 0.90 ± |
| intestines | 3.24 | 0.04 | 0.03 | 0.03 | 1.15 | 0.42 | 1.03 | 0.53 |
| Colon | 1.66 ± | 0.19 ± | 0.10 ± | 0.13 ± | 2.83 ± | 0.71 ± | 0.70 ± | 1.39 ± |
| | 0.69 | 0.06 | 0.02 | 0.02 | 0.35 | 0.09 | 0.48 | 1.06 |
| Feces | 2.56 ± | 0.40 ± | 0.13 ± | 0.15 ± | 0.59 ± | 2.01 ± | 0.73 ± | 0.55 ± |
| | 3.03 | 0.13 | 0.01 | 0.03 | 0.24 | 0.93 | 0.45 | 0.32 |
| Kidneys | 10.8 ± | 3.66 ± | 3.27 ± | 2.90 ± | 12.3 ± | 6.71 ± | 7.00 ± | 7.39 ± |
| | 3.02 | 0.58 | 0.32 | 0.34 | 0.72 | 1.08 | 1.31 | 1.63 |
| Bladder | 3.83 ± | 0.29 ± | 0.24 ± | 0.34 ± | 3.41 ± | 0.70 ± | 0.65 ± | 1.00 ± |
| | 1.54 | 0.03 | 0.08 | 0.03 | 0.90 | 0.21 | 0.22 | 0.27 |
| Uterus | 0.62 ± | 0.16 ± | 0.15 ± | 0.18 ± | 1.07 ± | 0.64 ± | 1.14 ± | 1.09 ± |
| | 0.18 | 0.06 | 0.02 | 0.03 | 0.45 | 0.33 | 0.54 | 0.69 |
| Gastroc. | 0.26 ± | 0.05 ± | 0.04 ± | 0.05 ± | 0.45 ± | 0.21 ± | 0.19 ± | 0.13 ± |
| Muscle | 0.06 | 0.01 | 0.02 | 0.01 | 0.27 | 0.14 | 0.09 | 0.05 |
| Bone | 0.22 ± | 0.06 ± | 0.07 ± | 0.09 ± | 0.34 ± | 0.17 ± | 0.51 ± | 0.54 ± |
| | 0.06 | 0.01 | 0.00 | 0.00 | 0.20 | 0.05 | 0.16 | 0.16 |
| Skin | 0.76 ± | 0.11 ± | 0.08 ± | 0.25 ± | 1.03 ± | 0.25 ± | 0.45 ± | 0.50 ± |
| | 0.34 | 0.01 | 0.02 | 0.04 | 0.80 | 0.22 | 0.19 | 0.29 |
| Brain | 0.08 ± | 0.01 ± | 0.01 ± | 0.01 ± | 0.21 ± | 0.03 ± | 0.03 ± | 0.04 ± |
| | 0.01 | 0.00 | 0.00 | 0.00 | 0.20 | 0.01 | 0.01 | 0.02 |
| Eyes | 0.26 ± | 0.05 ± | 0.05 ± | 0.07 ± | 0.24 ± | 0.06 ± | 0.10 ± | 0.09 ± |
| | 0.09 | 0.01 | 0.01 | 0.03 | 0.03 | 0.02 | 0.03 | 0.01 |

% IA/g: percent of injected activity per gram; Gastroc. muscle: gastrocnemius muscle.

### Expanding the Chemistry Towards Multi-Drug Painting and ADC Engineering

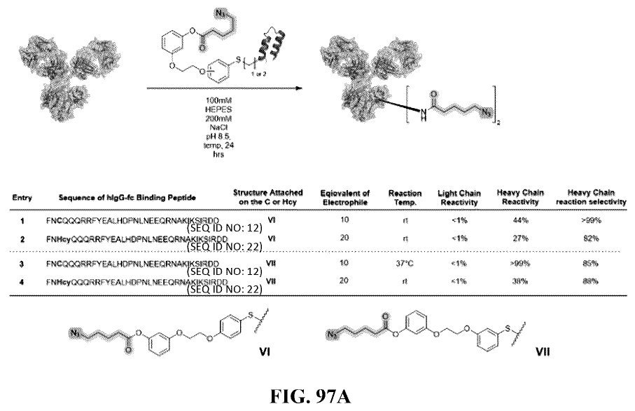

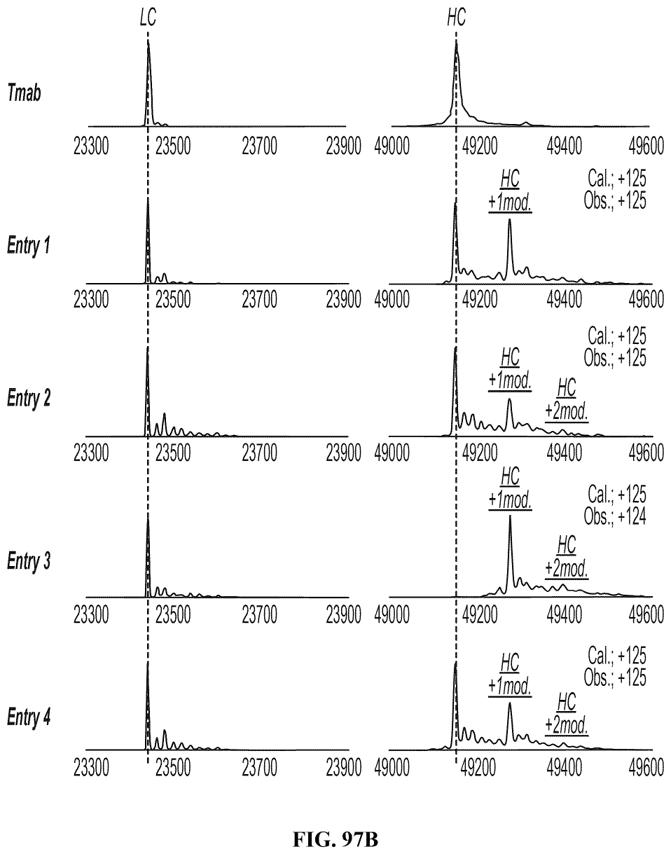

To enable the conjugation of two or more different drugs on one single IgG, two different strategies have been explored: (i) the site-specific modification of two different lysine side chains using different electrophilic warheads, (ii) the use of a bivalent electrophile. First, 5 novel Z33 variants (Table 16) were synthesized. For peptides 7,8, the L-Cys or L-Hcy residue substituted Met-3, then were conjugated to azido electrophiles VI and VII (FIGS. 97A to 100). Tmab sequencing indicated that azido conjugation occurred predominantly (˜93.63%) on Lys-248. Variants 9-11 carry a substitution of either Arg-31, or Asp-32 for the incorporation of electrophile VII. About 91% of Tmab heavy chain was successfully modified with two or more azido moieties while 86% of the light chain remained unaffected, highlighting the possibility to incorporate two different drug payloads via two steps serial reactions.

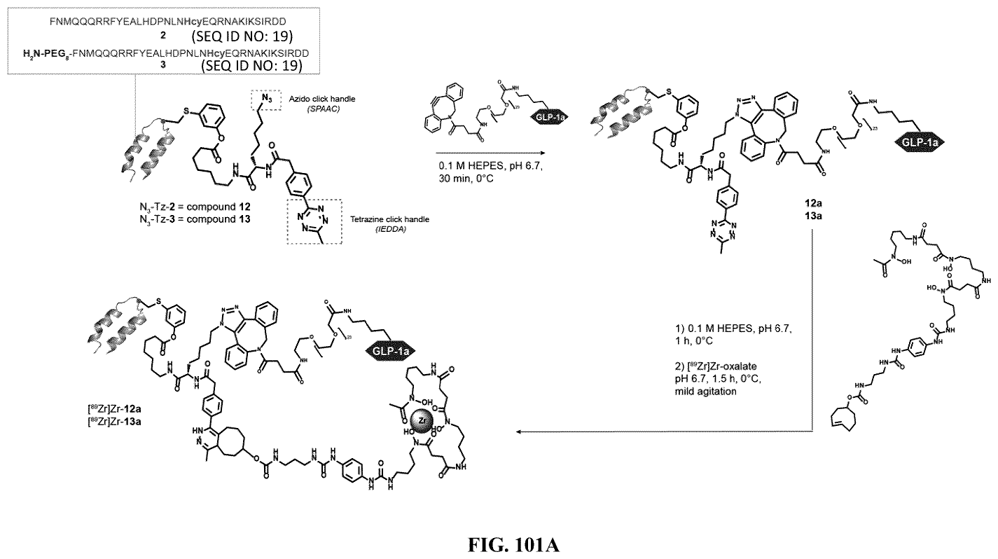

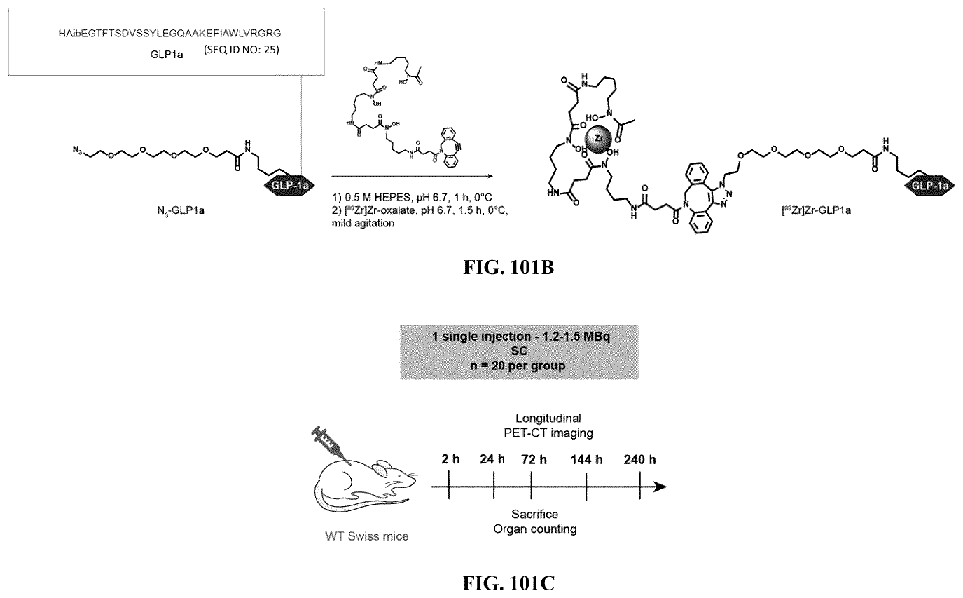

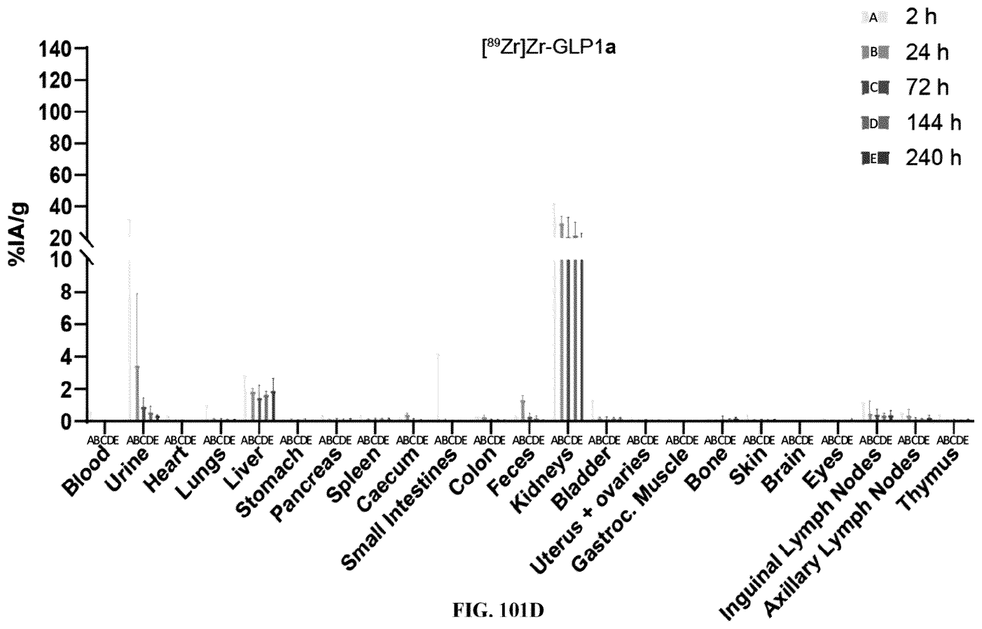

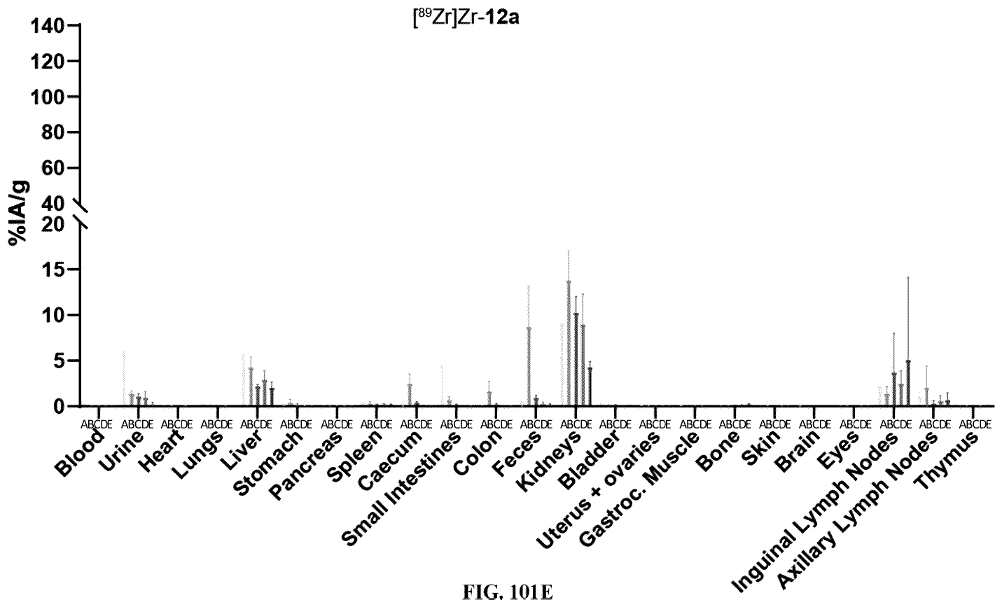

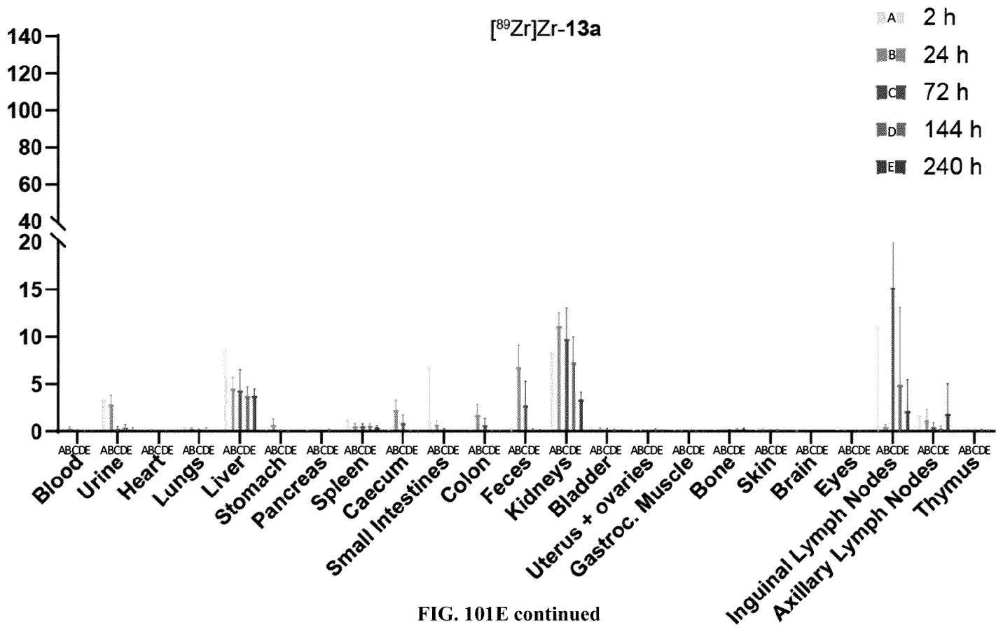

Multi-drug conjugation was then achieved through the linkage of peptides 2,3 to VIII, a dendronized electrophile bearing both an azido and a Tz moiety (FIGS. 101A to 101D). N₃-Tz-2,3 VIII was conjugated to DBCO-PEG₂₄-GLP-1a, then to TCO-DFO and radiolabeled with Zr-89, leading to [⁸⁹Zr]Zr-12a, 13a. After SC injection in WT female mice (1.2-1.5 MBq), a significant uptake was observed in the gut and in lymph nodes, confirming the reaction to mIgGs.

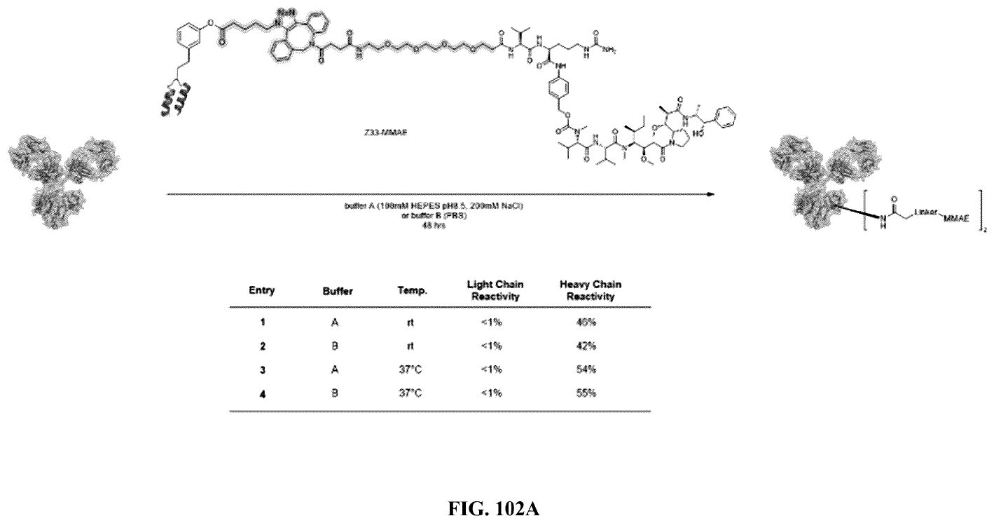

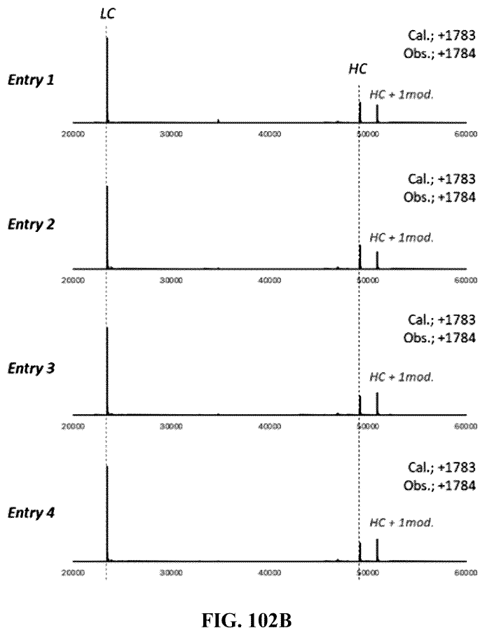

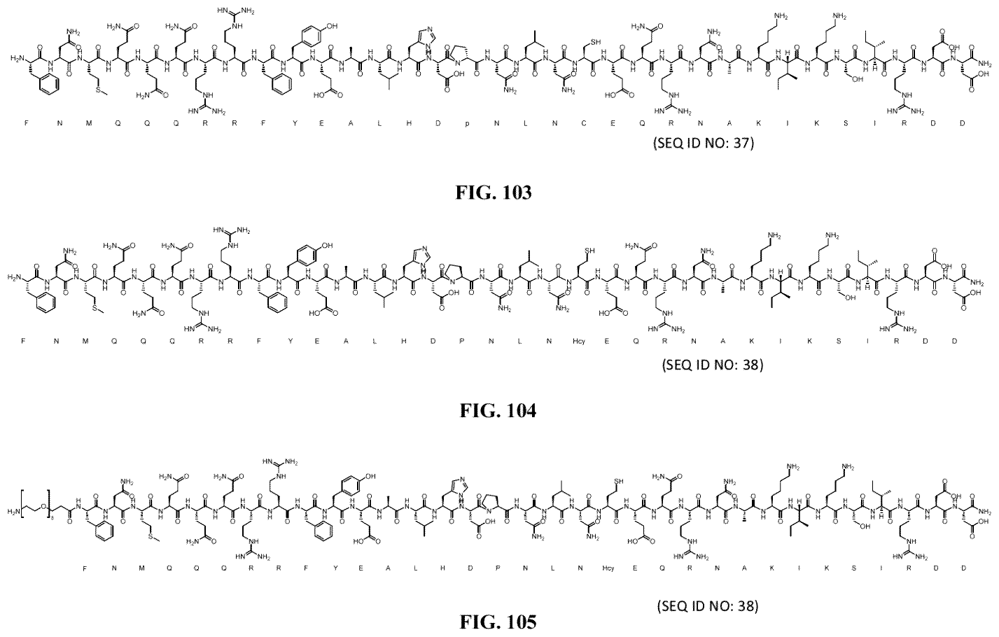

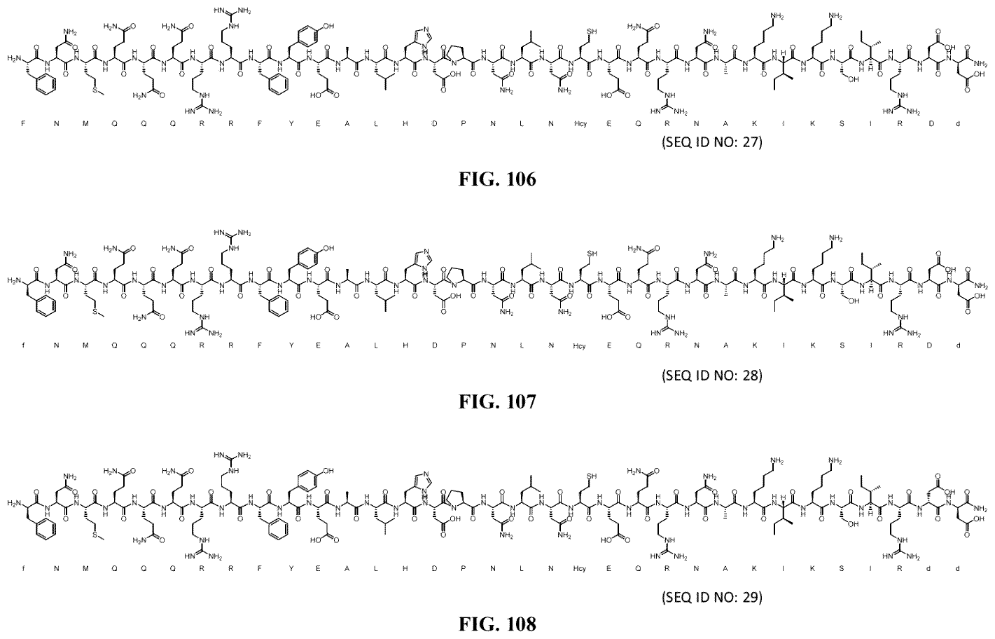

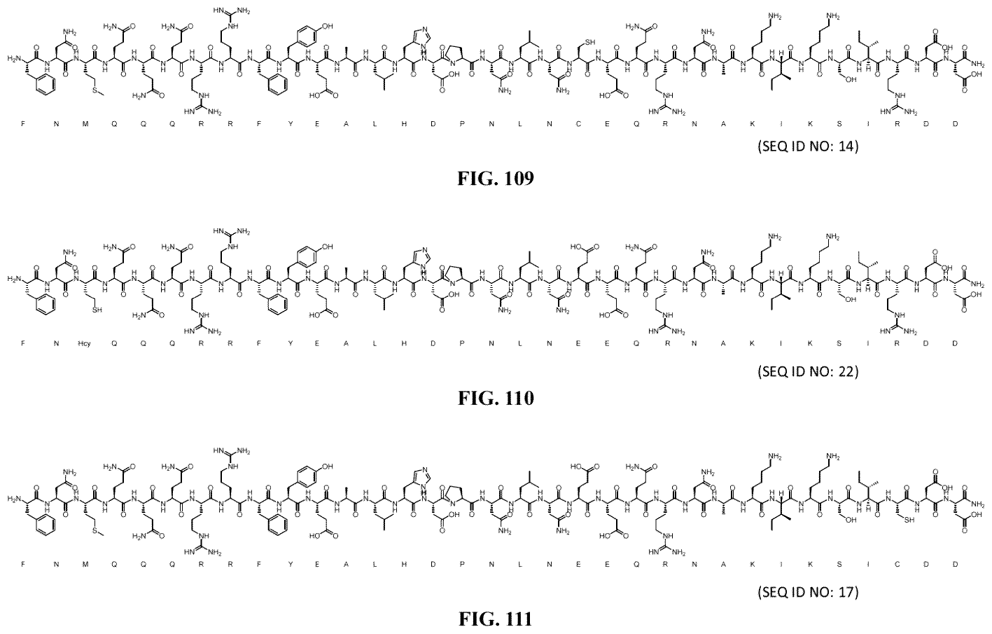

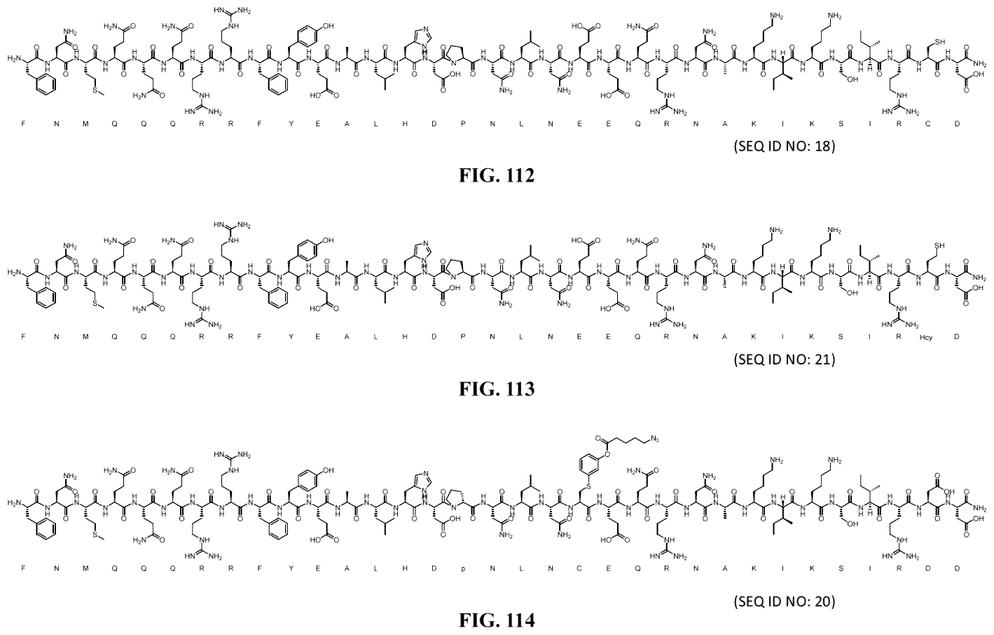

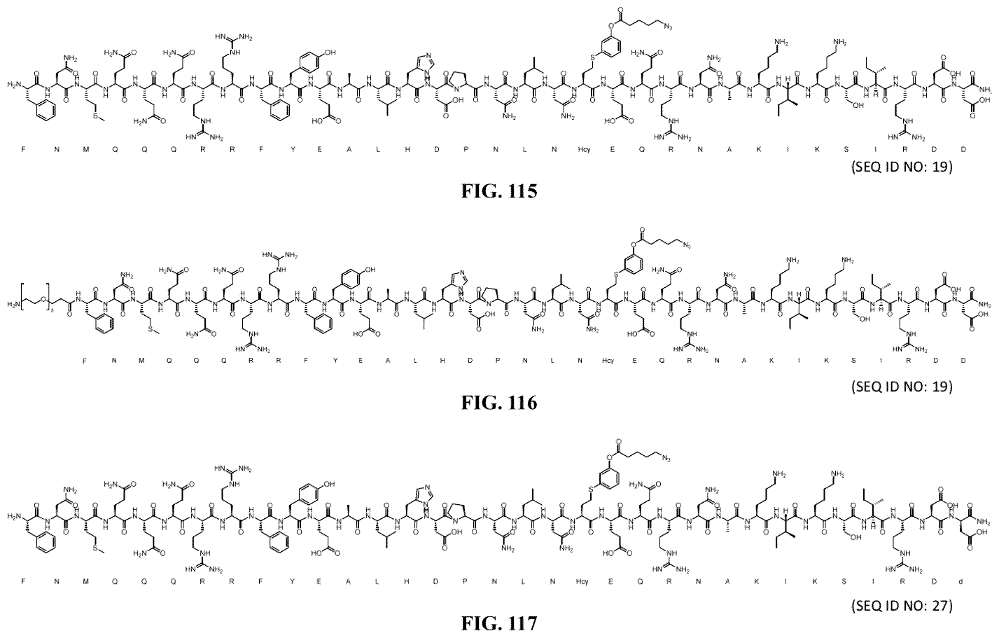

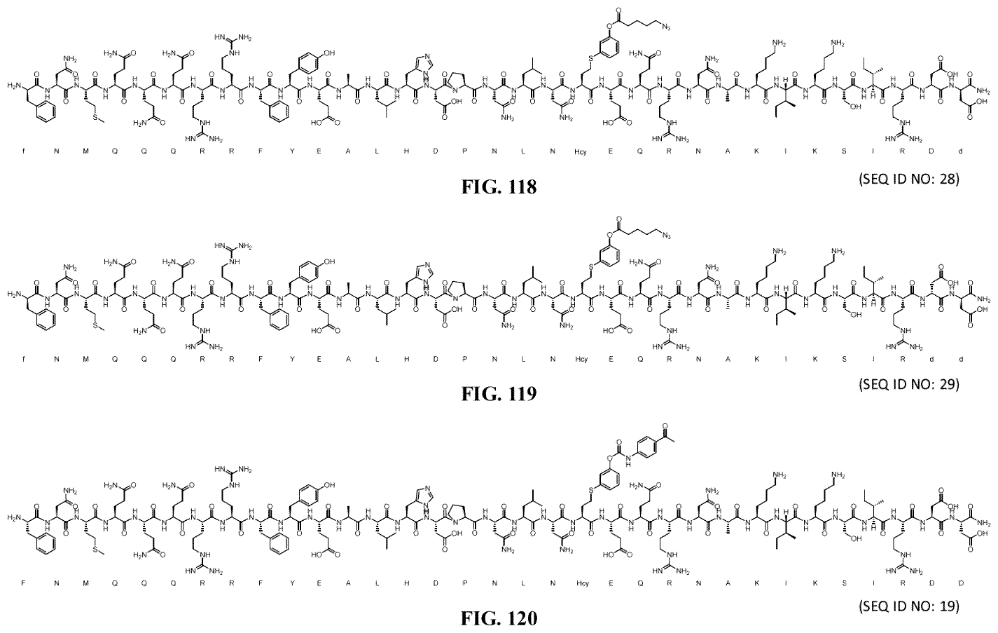

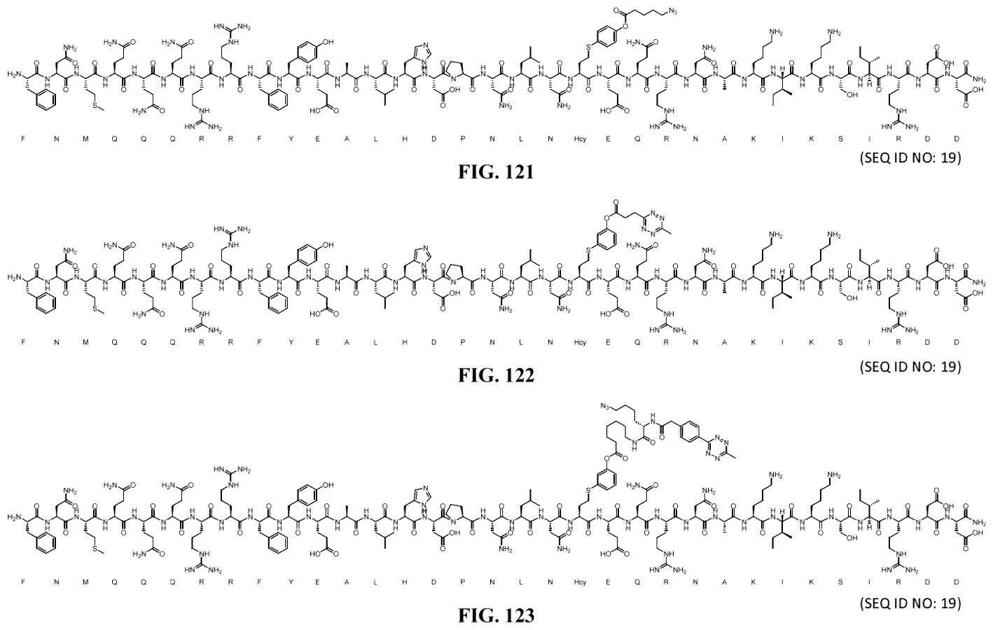

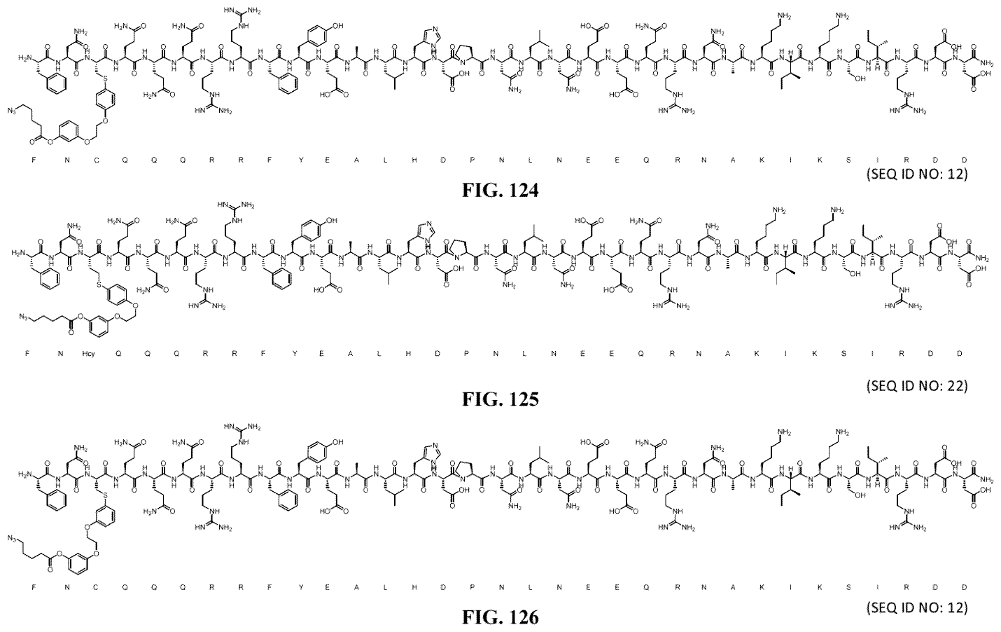

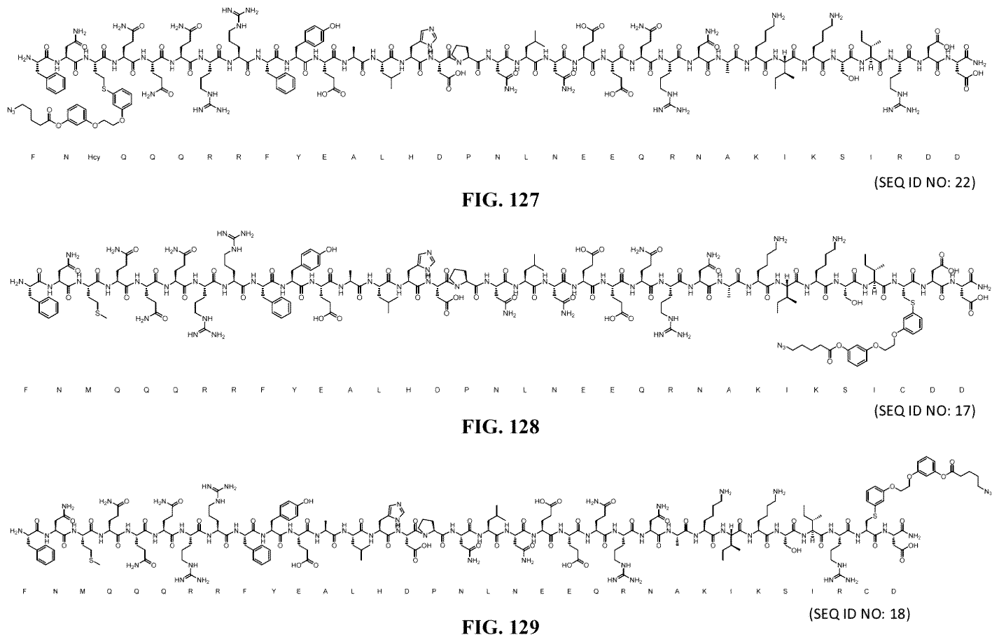

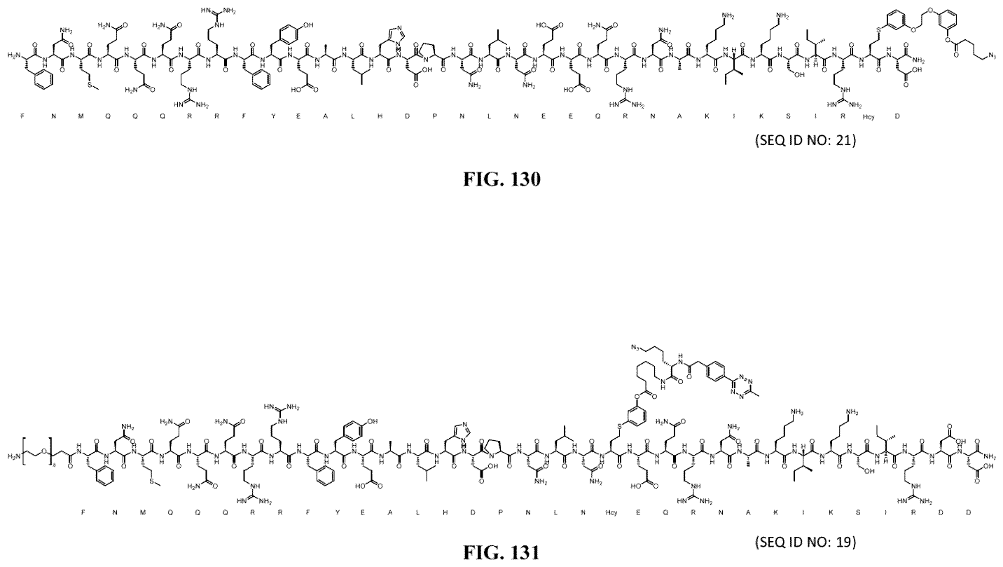

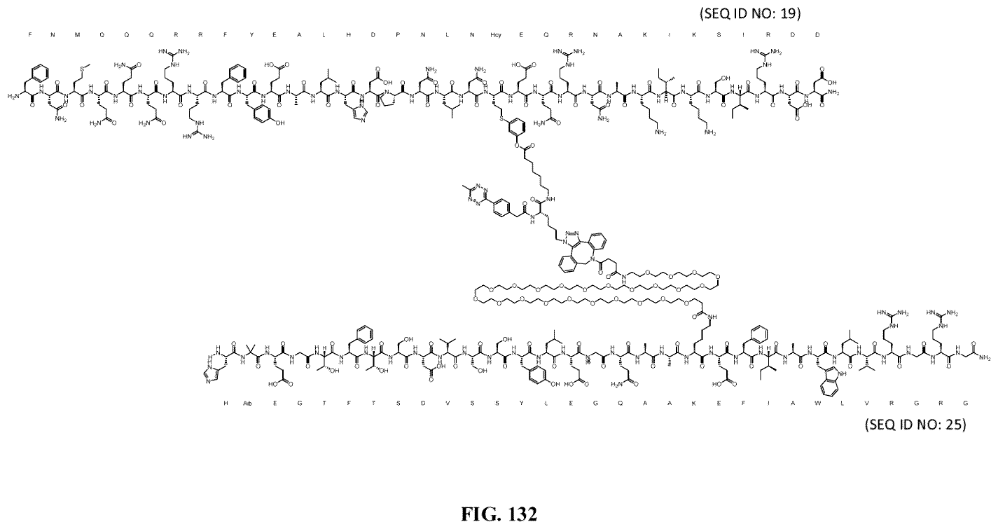

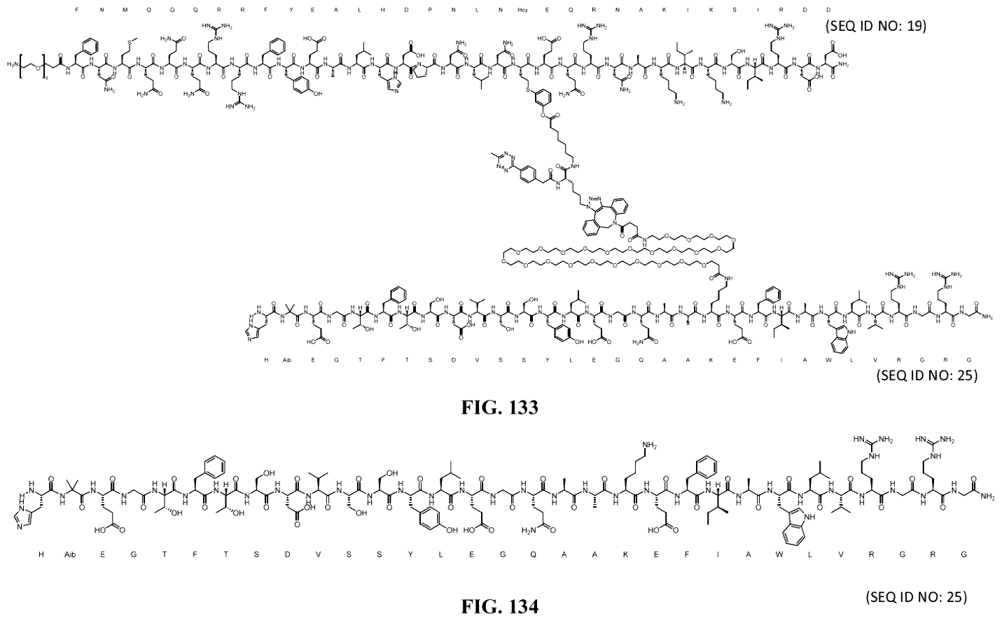

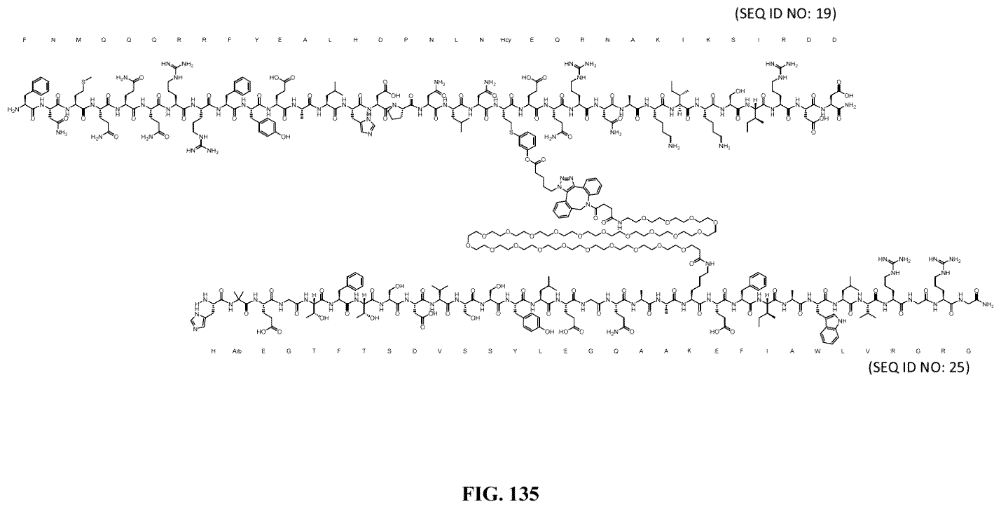

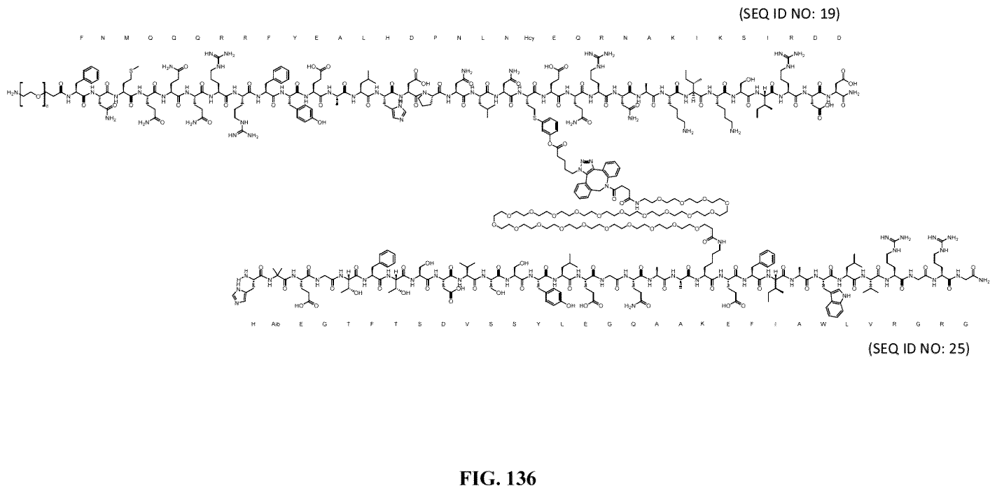

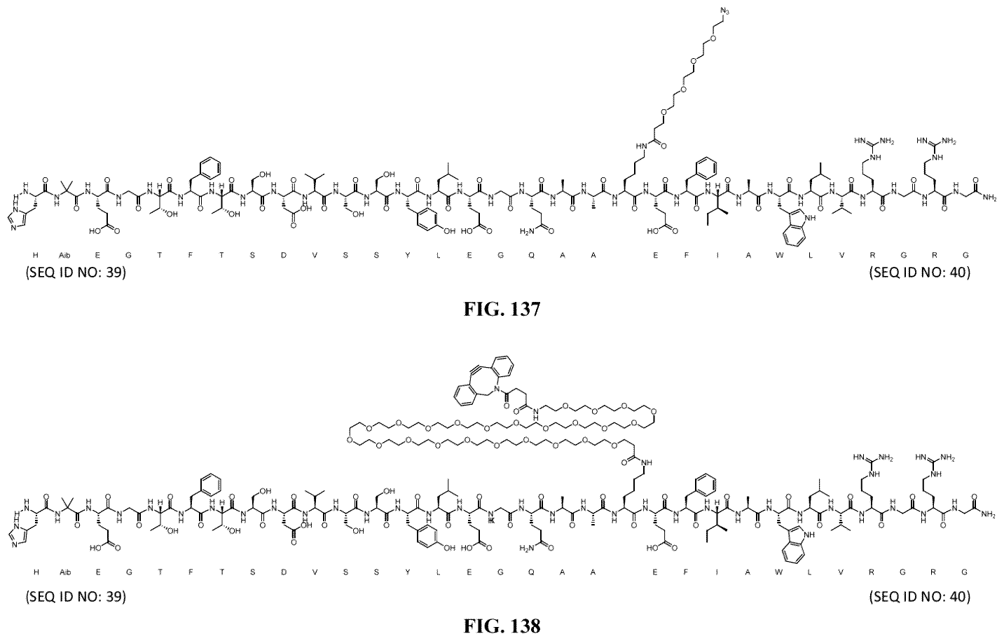

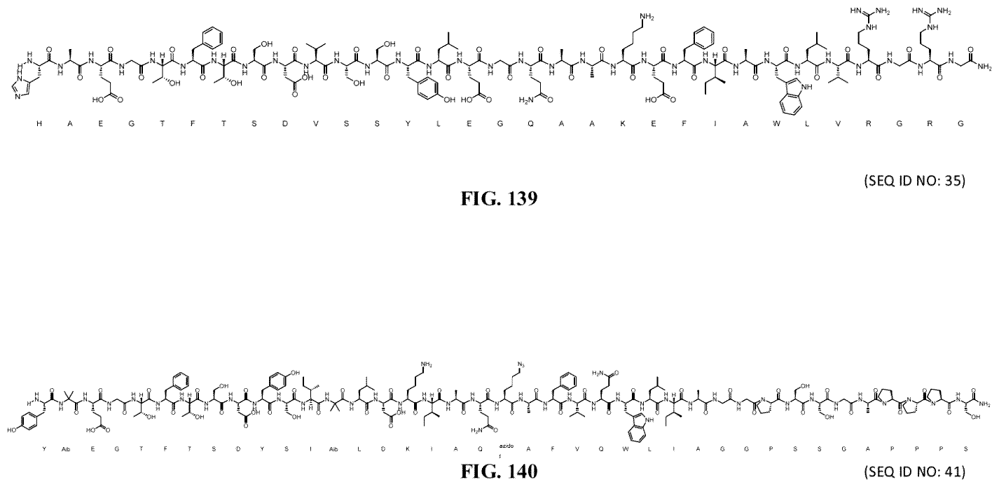

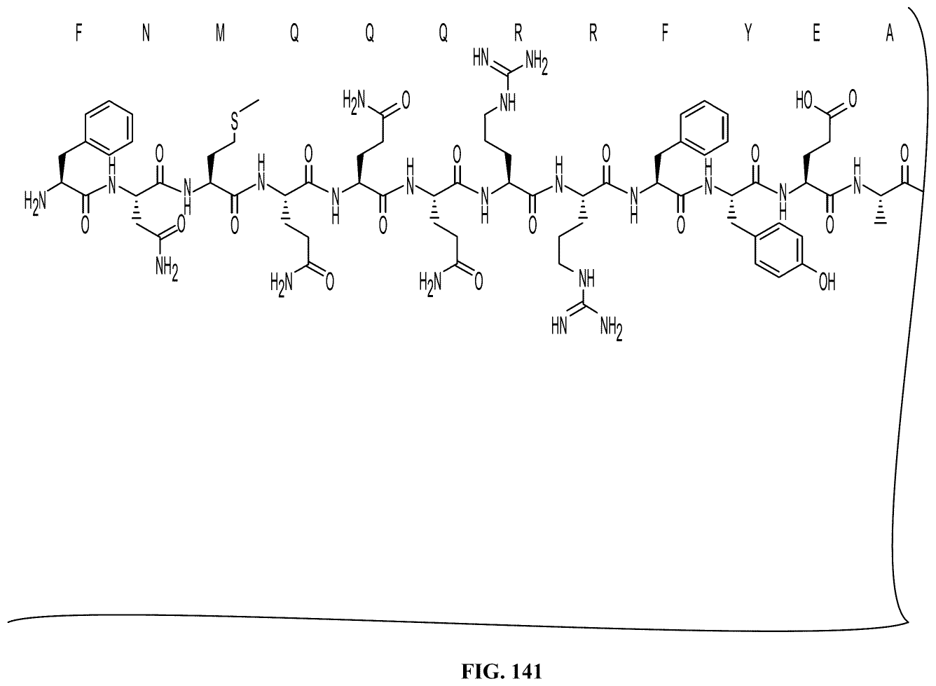

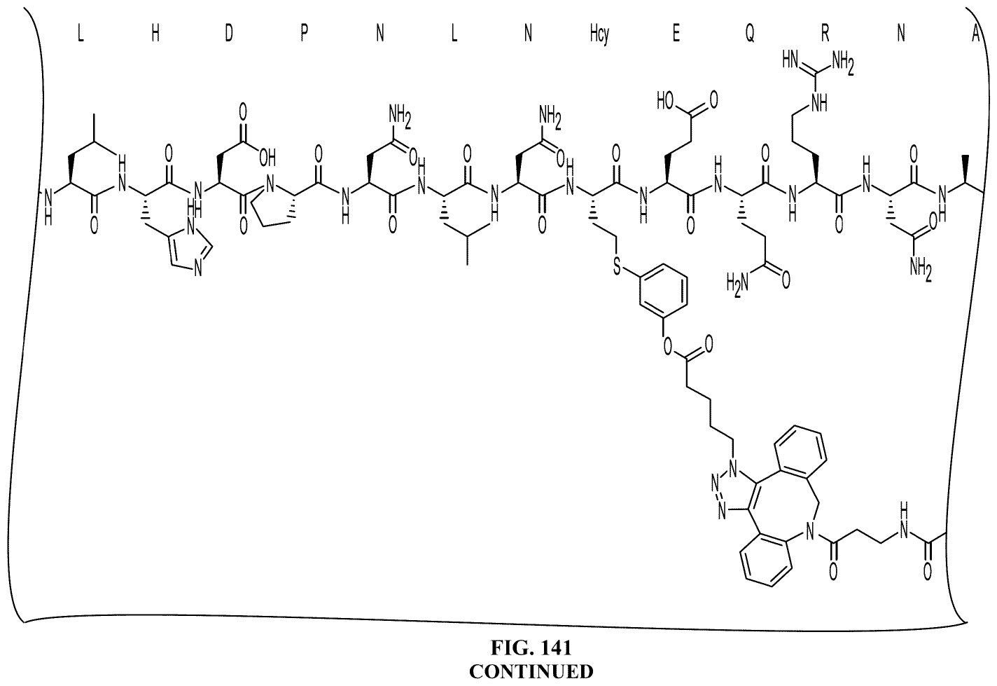

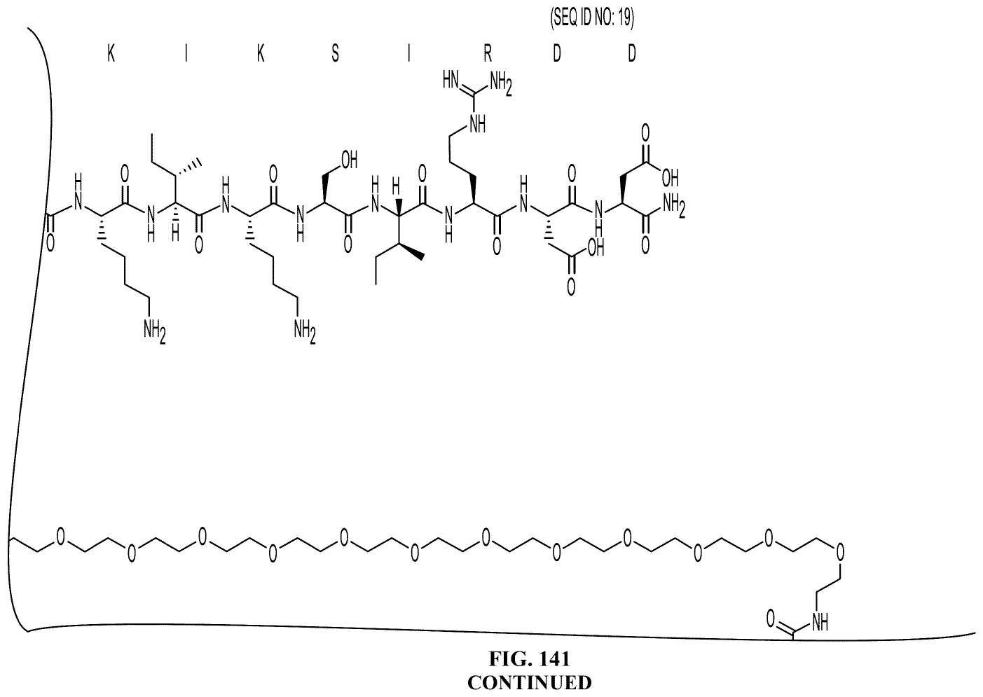

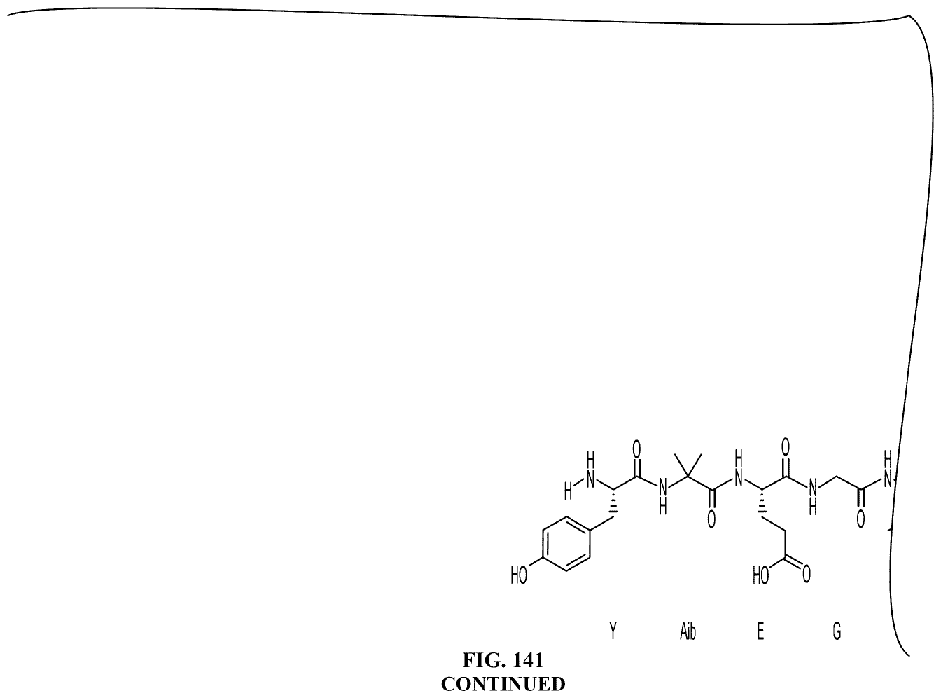

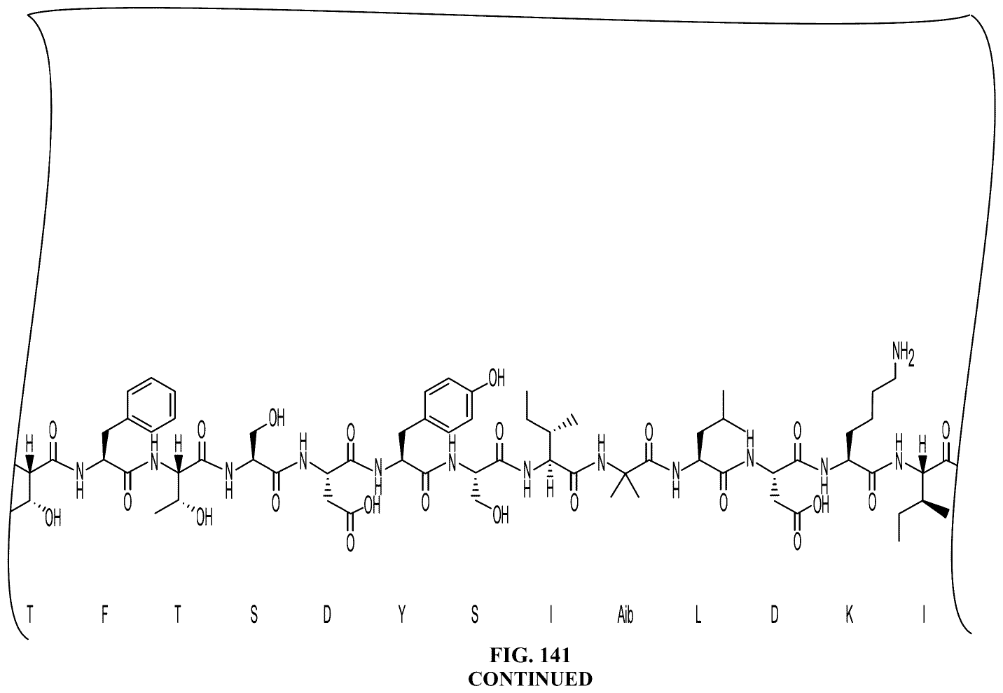

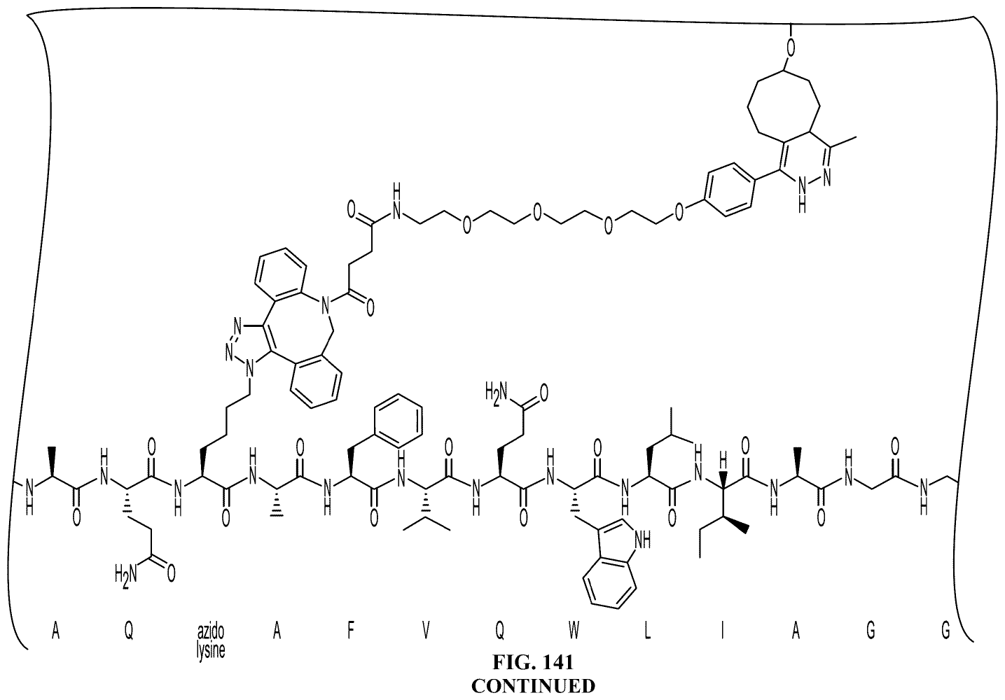

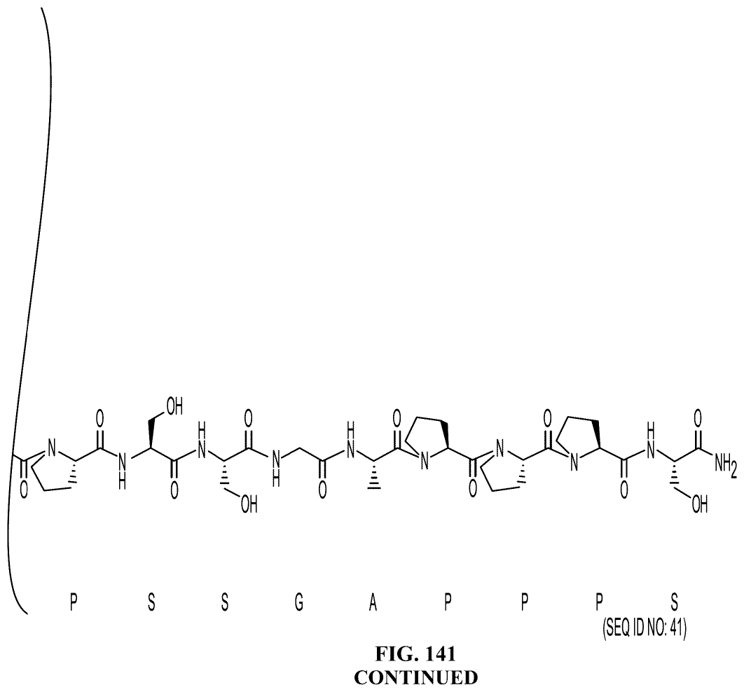

It is important to note that the drug delivery system can also be used in vitro, for engineering traditional antibody-drug conjugates (ADC), in a fast, site-selective, and highly reproducible manner (FIGS. 102A and 102B). Here monomethyl auristatin E (MMAE) was attached to Tmab, modifying up to 55% of the heavy chains of the Fc domain of Tmab with a DAR of 2 and a 99% selectivity. The the results highlight that this drug delivery platform can be used for a wide variety of applications.

### CONCLUSION

Fusion to FcRn binder molecules for PK extension purposes was explored in the past two decades and has already led to the approval of several drugs (18, 35). Applied to GLP-1 analogs, such approaches have provided T2DM patients an efficient therapeutic option and recently, FDA approval was obtained for weight loss applications (36, 37). A recent report on AMG-133 indicated that the fusion of two GLP-1 moieties on an anti-GIP IgG enabled body-weight loss in db/db mice after 24 h and persistent up to 216 h post IP injection of 2 mg/kg. In the same study, blood glucose was reduced up to 144 h post injection (15). In addition, in humans, AMG-133 was safe and tolerable besides an increase of amylase and lipase, possessed a half-life of 14-16 days with peak of duration reached 4-7 days post SC injection, and maintained body weight loss up to 120 days after the last dose. These data suggest that an antibody-GLP-1 conjugate may be effective at a twice monthly dosing regimen rather than the once-weekly semaglutide, with a significant lower toxicity. The manufacturing of antibodies is however time- and resource-intensive, and among the most costly steps in the entire production pipeline. Therefore, the in vivo antibody painting technology developed proposes an efficient, straightforward, and cost efficient alternative.

The synthesis of 13 different Fc-binder peptides, derived from the structure of Z33 is reported (21). Every compound was obtained with high purity and good yield, as a result of AFPS synthesis technology (20). Seven electrophilic groups were synthesized, conjugated to the Fc-binder peptides to evaluate their transfer reactivity towards the heavy chain Fc domain of Tmab, enabling us to determine the best Fc-binder peptide/electrophile pair for moving forward to in vivo experiments. The ability to transfer a cargo from a peptide to a native IgG while circulating in blood, in two hours has been successfully demonstrated, which is believed to have not been reported before.

The proof-of-concept of in vivo IgG painting with GLP-1 analogs was performed in WT mice and Lep^{ob/ob} (38) to evaluate the safety, the PK/PD extension, and the efficacy on obesity and TD2M. In both models a sustained body weight loss for 10 to 15 days, associated with blood glucose decrease for 144 h post SC injection, was successfully demonstrated. Moreover, in the obesity model, a better response to insulin was also measured after 10 days following either SC or IP injection. While the outcome was similar, slight sex-related differences were observed for the treatment response among the male and female obese mice, a pattern observed also for GLP-1 agonists in humans (39). Overall, in vivo IgG painting with GLP-1 achieved either similar or better results than the commercial semaglutide, at a dose 4 times lower (40, 41).

Finally, in vivo IgG painting can achieve site-selective modification of either one or two lysine side chains, from both sides of the heavy chain Fc domain depending on the electrophile used, making the conjugation of two different drugs possible on the same antibody. The development of long-acting GLP-1 analogs is currently moving towards the combination of two or more incretin co-agonists to boost the effect on T2DM and weight loss (16, 42). While the engineering of bispecific antibodies is challenging, the IgG painting platform, which can be performed either in vitro or in vivo, introduces a facile modality to access multi-drug antibody conjugates in the body.

### Material and Methods

### General Information

### Materials

Fmoc-L-Ala-OH, Fmoc-L-Arg(Pbf)-OH, Fmoc-L-Asn(Trt)-OH, Fmoc-L-Asp-(OtBu)-OH, Fmoc-L-Cys(Trt)-OH, Fmoc-L-Gln(Trt)-OH, Fmoc-L-Glu(OtBu)-OH, Fmoc-L-Gly-OH, Fmoc-L-His(Trt)-OH, Fmoc-L-Ile-OH, Fmoc-L-Leu-OH, Fmoc-L-Lys (Boc)-OH, Fmoc-L-Met-OH, Fmoc-L-Phe-OH, Fmoc-L-Pro-OH, Fmoc-L-Ser(tBu)-OH, Fmoc-L-Thr(tBu)-OH, Fmoc-L-Trp(Boc)-OH, Fmoc-L-Tyr(tBu)-OH, and Fmoc-L-Val-OH were purchased from Novabiochem, Millipore Sigma, or Chem-Impex Inc. Fmoc-alpha-methylalanine (Fmoc-Aib-OH) and Fmoc-L-HomoCys(Trt)-OH (Fmoc-Hcy-OH) were purchased from Combi-Blocks Inc. 5-Azidopentanoic acid was purchased from ChemPep®. 1-(9H-Fluoren-9-yl)-3-oxo-2,7,10,13,16,19,22,25,28-nonaoxa-4-azahentriacontan-31-oic acid (Fmoc-PEG8-CO2H) was purchased from AmBeed. O-(7-azabenzotriazol-1-yl)-N,N,N′,N′-tetramethyluronium hexafluorophosphate (HATU), and (7-azabenzotriazol-lyloxy)tripyrrolidinophosphonium hexafluorophosphate (PyAOP) were purchased from P3 Biosystems. H-Rink Amide-ChemMatrix resin was purchased from PCAS BioMatrix Inc. Polyethylene filter paper for peptide synthesis (0.60 mm thick, pore size 7-12 μm) was purchased from Interstate Specialty Products. N,N-Dimethylformamide (OmniSolv® for Biosynthesis, DMF, stored with an AldraAmine trapping packet), diethyl ether (≥99.0% stabilized with BHT, Et₂O), acetonitrile (≥99.9%, CH₃CN), dichloromethane (≥99.8% stabilized with amylene, CH₂Cl₂), 1,4-dioxan (≥99.5% stabilized by BHT), 2-propanol (99.9%), 2-methyl-tetrahydrofuran (≥99.5% stabilized with BHT, 2-Me-THF), dimethylsulfide (≥99.5%, BioUltra for molecular biology, DMSO), and pentane (98% reagent grade) were obtained from Millipore-Sigma. Ethyl acetate (≥99.5%), hexane (≥98.5%, mixture of isomers), methanol (≥99.8%), and acetone (≥99.5%) were purchased from VWR. The water used in all reactions involving proteins, in the preparation of buffers, and in the preparation of mobile phases for purification was obtained via filtration of deionized water through a Millipore Sigma Milli-Q Ultrapure Water System. AldraAmine trapping packets and piperidine were purchased from Millipore Sigma. Amine-free DMF refers to DMF stored over AldraAmine trapping packets for a minimum of 24 hours prior to use. LC/MS grade water, acetonitrile, and formic acid were purchased from Thermo Fisher Scientific Inc and were used for liquid chromatography-mass spectrometry (LC-MS). All small compounds were purchased from TCI America, Millipore Sigma, AmBeed, Matrix Scientific, or Combi-Blocks in purities ≥95%, and were used without further purification (unless otherwise noted). PBS buffer (1× and 10×) were purchased from CORNING®. Reactions monitored by analytical thin-layer chromatography (TLC) were carried out using glass-backed plates pre-coated with silica gel impregnated with a fluorescent indicator (254 nm). All deuterium solvents were purchased from Cambridge Isotope Laboratories, Inc. C18-ZipTips (0.6 μL) were purchased from Millipore Sigma. Zeba spin desalting columns were purchased from Thermo Fisher Scientific Inc. Trastuzumab and its biosimilar were purchased from Syd Labs Inc., MedChemExpress LLC., and Bio X Cell.

### Nuclear Magnetic Resonance Spectroscopy (NMR)

NMR spectra were recorded on a Bruker Avance III HD 400 MHz spectrometer. All 1H NMR chemical shifts are expressed in parts per million (ppm, δ scale) and are referenced to the residual proton in the NMR solvent (CDCl₃-d3: 7.26, DMSO-d6: 2.50). All ¹³C spectra recorded are proton decoupled. The ¹³C NMR chemical shifts are expressed in part per million (ppm, δ scale) and are referenced to the carbon resonance of the NMR solvent (CDCl₃-d3: 77.16, DMSO-d6: 39.52). ¹H NMR spectroscopic data are reported as follows: a chemical shift in ppm (multiplicity, coupling constants J (Hz), integration intensity, assigned number of protons in molecule). The multiplicities are abbreviated with s (singlet), br. s (broad singlet), d (doublet), t (triplet), q (quartet), and m (multiplet). In the case of combined multiplicities, the multiplicity with the larger coupling constant is stated first. The chemical shift of all signals is reported as the center of the resonance range, except in the case of multiplets, which are reported as ranges in chemical shift. All raw fid files were processed, and the spectra analyzed using the program MestReNova 14.2.3 from Mestrelab Research S. L. Copies of the ¹H and ¹³C spectra can be found at the end of this document (unless otherwise noted).

### Liquid Chromatography-Mass Spectrometry (LC-MS)

### LC-MS Using Quadrupole Time-of-Flight (Q-ToF)

LC-MS chromatograms and associated mass spectra were acquired using an Agilent Technologies 6550 Q-ToF LC-MS system. Solvent compositions are 0.1% formic acid in H₂O (solvent A) and 0.1% formic acid in acetonitrile (solvent B). For methods A, B, and C, a calibration solution containing m/z at 922.0098 constantly flowed through the system. The following LC-MS methods were used:

### Q-ToF LC-MS Method A

LC conditions: Zorbax 300SB C3 column: 2.1×150 mm, 5 μm, column temperature: 40° C., gradient: 0-1 min 1% B, 1-6 min 1-61% B, flow rate: 0.8 mL/min. MS conditions: positive electrospray ionization (ESI) extended dynamic mode in mass range 300-3000 m/z, temperature of drying gas=350° C., flow rate of drying gas=11 L/min, pressure of nebulizer gas=60 psi, the capillary, fragmentor, and octapole voltages were set at 4000, 175, and 750 V, respectively.

### Q-ToF LC-MS Method B

LC conditions: Zorbax 300SB C3 column: 2.1×150 mm, 5 μm, column temperature: 40° C., gradient: 0-1 min 1% B, 1-7 min 1-91% B, flow rate: 0.7 mL/min. MS conditions: positive electrospray ionization (ESI) extended dynamic mode in mass range 300-3000 m/z, temperature of drying gas=350° C., flow rate of drying gas=11 L/min, pressure of nebulizer gas=60 psi, the capillary, fragmentor, and octapole voltages were set at 4000, 175, and 750 V, respectively.

### Q-ToF LC-MS Method C

LC conditions: Zorbax 300SB C3 column: 2.1×150 mm, 5 μm, column temperature: 40° C., gradient: 0-1 min 1% B, 1-6 min 1-41% B, flow rate: 0.8 mL/min. MS conditions: positive electrospray ionization (ESI) extended dynamic mode in mass range 300-3000 m/z, temperature of drying gas=350° C., flow rate of drying gas=11 L/min, pressure of nebulizer gas=60 psi, the capillary, fragmentor, and octapole voltages were set at 4000, 175, and 750 V, respectively.

### Q-TOF LC-MS Method D

LC conditions: Aeris WIDEPORE C4 200 column: 2.1×150 mm, 3.6 μm, column temperature: 40° C., gradient: 0-2 min 1% B, 2-8 min 1-91% B, 8-10 min 91-95% B, flow rate: 0.3 mL/min. MS conditions: positive electrospray ionization (ESI) extended dynamic mode in mass range 100-1700 m/z, temperature of drying gas=200° C., flow rate of drying gas=14 L/min, pressure of nebulizer gas=55 psi, the capillary, fragmentor, and octapole voltages were set at 3500, 175, and 750 V, respectively.

### Q-TOF LC-MS Method E

LC conditions: Aeris WIDEPORE C4 200 column: 2.1×150 mm, 3.6 μm, column temperature: 40° C., gradient: 0-2 min 1% B, 2-7 min 1-61% B, 7-8 min 61-95% B, flow rate: 0.3 mL/min. MS conditions: positive electrospray ionization (ESI) extended dynamic mode in mass range 100-1700 m/z, temperature of drying gas=200° C., flow rate of drying gas=14 L/min, pressure of nebulizer gas=55 psi, the capillary, fragmentor, and octapole voltages were set at 3500, 175, and 750 V, respectively.

Data were processed using Agilent MassHunter Workstation Qualitative Analysis Version B.06.00 Software or Agilent MassHunter BioConfirm Software B.10.00. Deconvoluted masses of proteins were obtained using a maximum entropy algorithm. Unless otherwise depicted, the following parameters were used for deconvolution: Mass range is given in the experimental section for each peptide; Mass step was set to 1.00 Daltons; Baseline was set to Subtract baseline, Baseline factor as 7.00. The y-axis of all chromatograms shown in the FIGS. 77 to 102B represents the total ion current (TIC), and the inset of the mass spectrum corresponds to the deconvolution of the entire protein including peaks.

### LC-MS Using Single Quadrupole Mass Spectrometry

Mass spectra were obtained on an Agilent 6125B mass spectrometer attached to an Agilent 1260 Infinity LC. Solvent compositions are 0.1% formic acid in H₂O (solvent A) and 0.1% formic acid in acetonitrile (solvent B). The following LC-MS method was used:

LC conditions: Poroshell 120 SB C18: 2.1×50 mm, 2.7 μm, column temperature: 40° C., gradient: 0-1 min 10% B, 1-5 min 10-100% B, 5-6 min 100% B, 6-7 min 100-10% B, flow rate: 0.4 mL/min. MS conditions: positive electrospray ionization (ESI) extended dynamic mode in mass range 100-1500 m/z, temperature of drying gas=350° C., flow rate of drying gas=13 L/min, pressure of nebulizer gas=35 psi, the capillary, fragmentor, and octapole voltages were set at 4000, 70, and 650 V, respectively.

Nano-Liquid Chromatography-Tandem Mass Spectrometry (nLC-MS/MS)

Analysis was performed on an EASY-nLC 1200 nano-liquid chromatography system connected to an Orbitrap Fusion Lumos Tribrid Mass Spectrometer or to an Orbitrap Fusion Eclipse Tribrid Mass Spectrometer (Thermo Fisher Scientific). Samples were run on a PepMap RSLC C18 column (C18, 50 μm×15 cm, 2 μm, 100 Å, Thermo Fisher Scientific, P/N ES901). An Acclaim PepMap 100 Trap column (C18, 75 μm×2 cm, 3 μm, 100 Å, Thermo Fisher Scientific, P/N 164946) was used for desalting. Solvent compositions are 0.1% formic acid in H₂O (solvent A) and 0.1% formic acid in 80% acetonitrile and 19.9% H₂O (solvent B). The following conditions were used for each sample measurement:

nLC-MS/MS Method A

LC conditions: column temperature: 40° C., gradient: 0-30 min 30-95% B, 30-40 min 95% B, flow rate: 300 nL/min. MS conditions: positive ion spray voltage was set to 2200 V. Orbitrap detection was used for primary MS, with the following parameters: Application mode=peptide, cycle time=3 s, resolution=120000, mass range=normal, scan range=250-2000 m/z, RF Lens=30%, AGC target=standard, maximum injection time mode=auto, 1 microscan, data type=profile, polarity=positive. The following filters were applied for MS2 precursor selection: monoisotopic peak determination=peptide, filter type=threshold, Intensity threshold=5.0e5, include charge state=2-6, mass list type=m/z, target mass*, mass tolerance=25 ppm. Fragmentation was induced by higher-energy collisional dissociation (HCD) and electron-transfer dissociation with higher-energy collision (EThcD). Specifications HCD: isolation mode=quadrupole, isolation window=1.2 m/z, isolation offset=0.6 m/z, activation type=HCD, collision energy mode=fixed (30%), detection type=orbitrap, resolution=30000, mass range=normal, first mass=120 m/z, AGC target=standard, maximum injection time mode=dynamic, microscan, data type=profile. Specifications EThcD: isolation mode=quadrupole, isolation window=1.2 m/z, isolation offset=0.6 m/z, activation type=ETD, use calibrated charge-dependent ETD parameters, ETD supplemental activation=EThcD, SA collision energy=30%, detection=orbitrap, resolution=30000, mass range=normal, first mass=120 m/z, normalized AGC target=standard, maximum injection time=dynamic, 1 microscans, data type=profile.
*A list of target masses was created by calculating the intrinsic masses of the measured samples.
   nLC-MS/MS Method B

LC conditions: column temperature: 40° C., gradient: 0-30 min 1-10% B, 30-120 min 10-81% B, 120-125 min 81-90% B, 125-135 min 90% B, flow rate: 300 nL/min. MS conditions: positive ion spray voltage was set to 2200 V, and negative ion spray voltage was set to 600 V. Orbitrap detection was used for primary MS, with the following parameters: Application mode=peptide, cycle time=3 s, resolution=120000, mass range=normal, scan range=200-1400 m/z, RF Lens=30%, AGC target=custom, normalized AGC target (%)=250, maximum injection time mode=auto, 1 microscan, data type=profile, polarity=positive. The following filters were applied for MS2 precursor selection: monoisotopic peak determination=peptide, exclude after 4 times, If occurs within 30 s, exclusion duration=30 s, mass tolerance=10 ppm, exclude isotope, include charge state=2-10, filter type=threshold, Intensity threshold=5.0e4, precursor selection range=200-1400, Fragmentation was induced by collision-induced dissociation (CID), higher-energy collisional dissociation (HCD), and electron-transfer dissociation with higher-energy collision (EThcD). Specifications CID: isolation mode=quadrupole, isolation window=1.3 m/z, activation type=CID, collision energy mode=fixed (30%), CID activation time=10 ms, activation Q=0.25, detection type=orbitrap, resolution=30000, mass range=normal, AGC target=custom, normalized AGC target=40%, maximum injection time mode=auto, 1 microscan, data type=centroid. Specifications HCD: isolation mode=quadrupole, isolation window=1.3 m/z, activation type=HCD, detection type=orbitrap, resolution=30000, mass range=normal, scan range mode=auto, AGC target=custom, normalized AGC target=40%, maximum injection time mode=auto, 1 microscan, data type=centroid. Specifications EThcD: isolation mode=quadrupole, isolation window=1.3 m/z, activation type=ETD, use calibrated charge-dependent ETD parameters, ETD supplemental activation=EThcD, SA collision energy=25%, detection=orbitrap, resolution=30000, mass range=normal, scan range mode=auto, normalized AGC target=40%, normalized AGC target=custom, maximum injection time=auto, 1 microscans, data type=profile.

### Ultra High-Performance Liquid Chromatography (UHPLC)

The samples were analyzed using an Agilent Technologies 1290 Infinity II LC system which was computer-controlled through Agilent ChemStation software. The following methods were used:

### UHPLC Method

Solvent compositions used in the UHPLC are 0.1% TFA in H₂O (solvent A) and 0.1% TFA in acetonitrile (solvent B). ACQUITY UPLC Protein BEH C4 column: 2.1×50 mm, 1.7 μm (Waters, P/N 186004495), ACQUITY UPLC Protein BEH C4 VanGurde pre-column: 2.1×5 mm, 1.7 μm (Waters, P/N 186004623), column temperature: 27° C., gradient: 0-3 min 1% B, 3-13 min 1-61% B, flow rate: 0.5 mL/min.

### Automated Liquid Chromatography Purification

A Biotage® Selekt automated flash chromatography system was used for the purification of small molecules and peptides. The columns, solvents, and solvent gradients employed for the purification of individual compounds are written in the synthesis section of each compound.

### Sodium Dodecyl Sulfate-Polyacrylamide Gel Electrophoresis (SDS-PAGE)

Bolt 4-12% Bis-Tris Plus 1.0 mm×15 well or 1.0 mm×10 well plates (Invitrogen), Mini Gel Tank (Invitrogen) along with the PowerPac HC (BIO-RAD) were used for SDS-PAGE analysis. SeeBlue® Plus2 standard (Invitrogen) was used as the molecular weight standard. Gels were run using Bolt MOPS SDS Running Buffer (1×, Invitrogen) under the conditions of 135 V for 55 min. After electrophoresis, the running buffer was discarded, and the gel was placed in deionized water. Subsequently, the water with gel was heated in a microwave for 1 min and 30 s. The heated water was replaced, and the gel was thoroughly rinsed. Gel staining was performed for 10 min using SimpleBlue SafeStain (Invitrogen). The stain was removed following staining, and the gel was immersed in deionized water. The tray with gel and deionized water was placed on a shaker overnight to remove the stain from the gels. Then, ChemDoc MP Imaging System (BIO-RAD) was used to analyze the stained gels.

### Determination of the Reaction Conversions of IgGs

The conversion rate for each reaction was calculated by one of the following methods.

### Determination by LC-MS

Reported yields based on LC-MS spectra were determined by extracting the total ion current (TIC) spectra of all protein-containing species in the chromatogram utilizing Agilent MassHunter 6.0 software with the BioConfirm package. These extracted chromatograms were deconvoluted utilizing a maximum entropy algorithm and abundance of each species determined using total ion count.

### Determination by SDS-PAGE

Band densitometry was calculated using ImageJ software (imagej.nih.gov/ij/).

Densitometry-based yields were calculated based on the product: starting material protein ratio in lanes and standardized based on molecular weight.

After plotting bands and integration using ImageJ software, the value of each band was obtained as 7122.93 and 10408.80, respectively.

Starting material: right peak standardization (MW 49151) 10408.80/49151=0.21177

Modified product: left peak standardization (MW 53381) 7122.93/53381=0.13344

Thus, the determined conversion (%) corresponds to Standardized Product/(Standardized Total): 0.13344/(0.21177+0.13344)*100=39%

Chemical Synthesis of IgG Binding Peptide Z33 Analogs Automated Flow-based Peptide Synthesis System

The synthesis of the IgG binding peptide Z33 analogs were performed using the Automated fast-flow solid phase peptide synthesis (AFPS) system assembled in the Pentelute lab (20, 21). The synthesis conditions are summarized in the following table.

| Parameter | Conditions |
|---|---|
| temperature | 85-90° C. in reactor, 60° C. in 5′ activation loop (for C |
| | and H), 90° C. in 10′ activation loop (for all other |
| | amino acids) |
| Flow Rate | 40 mL/min |
| Coupling Step | 0.40 M amino acids stocks in DMF |
| | 0.38 M activator stocks in DMF |
| | Coupling conditions: HATU (13 pump strokes) except |
| | S and A with HATU (26 pump strokes) and C, H, N, |
| | Q, V, R, T with PyAOP (26 pump strokes) |
| Deprotection | 40% piperidine in DMF with 2% formic acid (13 pump |
| Step | strokes) |
| Washing Steps | DMF (40 pump strokes) |

### Synthesis of Z33 Variants Composed of Natural Amino Acids

A plastic fritted syringe (6 mL) was equipped with polyethylene filter paper, and ChemMatrix® H-Rink Amide resin (0.49 mmol/g, 150 mg) was loaded onto it. The syringe assembly was then placed on a manifold. The resin was swollen in DMF, and subsequently, a homogeneous resin slurry was prepared by repeatedly drawing back 500 μL of the slurry using a 1 mL pipette. Next, the syringe was set to the AFPS system for peptide synthesis. After completion of the synthesis, the syringe was transferred from the AFPS system to the manifold and washed three times with CH₂Cl₂. To minimize methionine oxidation in the peptide sequence, drying of the resin was not conducted using air flow on the manifold but rather using nitrogen flow. Specifically, the syringe was capped with a rubber septum, and a needle connected to a nitrogen flow line was inserted into the septum for 15 min. The resulting peptide-bound dry resin was subjected to subsequent steps of cleavage and purification.

### Synthesis of Z33 Variants Containing Unnatural Amino Acids

The peptide was synthesized using the method described in the “Synthesis of Z33 Variants Composed of Natural Amino Acids” section from the C-terminus of the sequence to the position of the unnatural amino acid. The resin in a plastic fritted syringe was transferred from the AFPS system to a manifold. Fmoc-protected unnatural amino acid (10 equivalents for the peptide on the resin) in HATU/DMF (0.38 M, 9.5 equivalents) solution and DIEA (15 equivalents) were added to the resin and allowed to react for 50 min at room temperature. The reaction solution was stirred with a spatula every 10 min. After the reaction, the reaction solution was removed, and the resin in the syringe was washed three times with 5 mL of DMF. Then, 3 mL of 40% piperidine/DMF solution (+2% formic acid) was added and allowed to react for 10 min. The reaction solution was removed, and 3 mL of 40% piperidine/DMF solution (+2% formic acid) was added again and allowed to react for 10 min. The reaction solution was removed, and the resin was washed three times with 4 mL of DMF. Addition of the subsequent natural amino acids to the peptide chain was again performed according to the procedure described in the “Synthesis of Z33 Variants Composed of Natural Amino Acids” section.

### Capping of the N-terminus of Z33 Variants.

After synthesis of the peptide by AFPS, the fritted syringe with resin was placed on a manifold. A HATU/DMF solution (0.38 M, 9.5 equivalents) of the carboxylic acid corresponding to the acyl group (10 equivalents for the peptide on the resin) and DIEA (15 equivalents) were added and allowed to react for 50 min at room temperature. During that time, the reaction solution was stirred with a spatula every 10 min. After the reaction, the reaction solution was removed, and the resin in the syringe was washed three times with 10 mL of DMF. If the introduced acyl group contained the Fmoc protecting group, 3 mL of 40% piperidine/DMF solution (+2% formic acid) was added and allowed to react for 10 min. The reaction solution was removed, and 3 mL of 40% piperidine/DMF solution (+2% formic acid) was added again and allowed to react for 10 min. The reaction solution was removed and washed three times with 4 mL of DMF and three times with CH₂Cl₂. To minimize methionine oxidation in the peptide sequence, the resin was not dried using airflow on the manifold but rather using nitrogen flow. Specifically, the syringe was capped with a rubber septum, and a needle connected to a nitrogen flow line was inserted into the septum for 15 min. The resulting peptide-bound dry resin was subjected to subsequent steps of cleavage and purification.

Cleavage of Z33 Variants from Resin

The peptide-bound resin was transferred to a 15 mL centrifuge tube. Subsequently, a 7.5 mL peptide cleavage solution (a mixture of TFA, water, EDT, and TIPS in a ratio of 94:2.5:2.5:1 by volume) was added to the centrifuge tube, and the reaction was allowed to proceed at room temperature for 2 hours on the shaker. Afterward, 5 mL of a plastic fritted syringe was placed on top of a 50 mL centrifuge tube, and the reaction solution containing the resin was filtered. The resin inside the syringe was washed twice with 3 mL of TFA. To the resulting solution, diethyl ether pre-cooled at −80° C. was added until a total volume of 45 mL was reached. The centrifuge tube was then capped, and the reaction solution was vortexed for 5 s, followed by centrifugation at 3220 rcf (relative centrifugal force) for 4 min. Subsequently, the supernatant was removed by decanting. The obtained crude peptide was dissolved in 10 mL of 50:50 water and acetonitrile solution (+0.1% TFA), followed by freezing the solution with liquid nitrogen and subjecting it to lyophilization. When the peptide contained the azido moiety, another peptide cleavage solution (a mixture of TFA, water, thioanisole, and TIPS in a ratio of 94:2.5:2.5:1 by volume) was used instead of the solution described above.

Purification of the Z33 Variants from Resin

The crude Z33 analog was dissolved in a 95:5 water-acetonitrile solution and loaded onto 12 g of Biotage® Sfär C18 column. The gradient used for purification was as follows: 3 CV (column volume) 5% B, 1 CV 5-10% B, 30 CV 10-40% B. Fractions containing the desired product were collected in 50 mL centrifuge tubes, and the solution was frozen using liquid nitrogen. The target Z33 variant was obtained as white solids by removing the solvent through lyophilization.

| | | | SEQ | | Calculated | Observed |
|---|---|---|---|---|---|---|
| | | | ID | | Mass | Mass |
| Code | N-term | Sequence | NO | C-term | (Da) | (Da) |
| 1 | Free | FNMQQQRRFYEALHDpNLNCEQRNA KIKSIRDD | 20 | Amide | 4078.6 | 4078.6 |
| 2 | Free | FNMQQQRRFYEALHDPNLNHcyEQR NAKIKSIRDD | 19 | Amide | 4092.6 | 4093.1 |
| 3 | 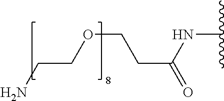 | FNMQQQRRFYEALHDPNLNHcyEQR NAKIKSIRDD | 19 | Amide | 4516.1 | 4516.2 |
| 4 | Free | FNMQQQRRFYEALHDPNLNHcyEQR NAKIKSIRDd | 27 | Amide | 4092.6 | 4092.5 |
| 5 | Free | fNMQQQRRFYEALHDPNLNHcyEQRN AKIKSIRDd | 28 | Amide | 4092.6 | 4092.5 |
| 6 | Free | fNMQQQRRFYEALHDPNLNHcyEQRN AKIKSIRdd | 29 | Amide | 4092.6 | 4092.6 |
| BB-1 | Free | FNMQQQRRFYEALHDPNLNCEQRNA KIKSIRDD | 14 | Amide | 4078.6 | 4078.5 |
| BB-2 | Free | FNCQQQRRFYEALHDPNL NEEQRNA KIKSIRDD | 12 | Amide | 4076.5 | 4077.1 |
| BB-3 | Free | FNHcyQQQRRFYEALHDPNLNEEQRN AKIKSIRDD | 22 | Amide | 4090.5 | 4090.7 |
| BB-4 | Free | FNMQQQRRFYEALHDPNLNEEQRNA KIKSICDD | 17 | Amide | \|4051.5 | 4052.3 |
| BB-5 | Free | FNMQQQRRFYEALHDPNLNEEQRNA KIKSIRCD | 18 | Amide | 4092.6 | 4092.7 |
| BB-6 | Free | FNMQQQRRFYEALHDPNLNEEQRNA KIKSIRHcyD | 21 | Amide | 4106.6 | 4106.5 |

### Synthesis of Small Molecule Reagents

Synthesis of Electrophiles with Allyl Halides

### Synthesis of BB-7

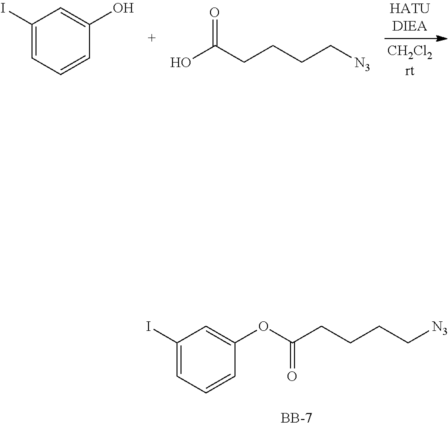 [SMILES: Oc1cccc(I)c1 ; [N-]=[N+]=NCCCCC(=O)O ; [N-]=[N+]=NCCCCC(=O)Oc1cccc(I)c1 ; "BB-7"]

A 100 mL round-bottom flask equipped with a stir bar was charged with 3-iodophenol (400 mg, 1.82 mmol), followed by the addition of 8 mL of CH₂Cl₂ to provide a clear solution. Next, 5-azidopentanoic acid (200 mg, 1.40 mmol), DIEA (0.973 μL, 5.59 mmol), and HATU (797 mg, 2.10 mmol) were added, and the resulting mixture was stirred at rt for 48 hours. The resulting mixture was quenched with saturated NaHCO₃ solution (100 mL) and partitioned between CH₂Cl₂ (200 mL) and water (100 mL). The organic phase was collected, and the aqueous phase was extracted with CH₂Cl₂ (100 mL) twice. The combined organic extract was washed with brine (50 mL), dried over sodium sulfate, filtered, and concentrated under reduced pressure. The obtained residue was purified by column chromatography (using Biotage® Sfär Silica 60 μm 25 g column, 95:5 to 3:1 hexane:ethyl acetate v/v) and dried under reduced pressure, yielding the desired compound as a clear oil (269 mg, 78%).

¹H NMR (400 MHZ, CDCl₃): δ 7.57 (dt, J=7.5, 1.5 Hz, 1H), 7.46 (t, J=1.8 Hz, 1H), 7.15-7.03 (m, 2H), 3.35 (t, J=6.6 Hz, 2H), 2.60 (t, J=7.3 Hz, 2H), 1.90-1.78 (m, 2H), 1.79-1.66 (m, 2H).

¹³C NMR (101 MHz, CDCl3): δ 171.27, 150.95, 135.04, 130.81, 130.79, 121.19, 93.62, 51.08, 33.69, 2.

HRMS (DART) m/z calcd for C₁₁H₁₃O₂N₃I ([M+H]⁺) 346.0047, found 346.0052.

### Synthesis of BB-8

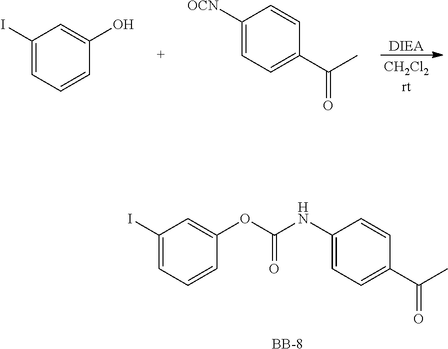

A 50 mL round-bottom flask equipped with a stir bar was charged with 3-iodophenol (324 mg, 1.47 mmol), followed by the addition of 7 mL of CH₂Cl₂ to provide a clear solution. Next, 4-acetylphenyl isocyanate (250 mg, 1.55 mmol) and DIEA (330 μL, 1.86 mmol) were added, and the resulting mixture was stirred at rt for 1 hour. The resulting mixture was then treated with diethyl ether (4 mL) and subjected to sonication for approximately 30 s. The reaction solution was filtered using a Büchner funnel, and the resulting solid was washed with dichloromethane (1 mL). Finally, the solid was dried under reduced pressure, yielding the desired compound as a white solid (241 mg, 43%).

¹H NMR (400 MHZ, DMSO): δ 10.68 (s, 1H), 7.95 (d, J=8.8 Hz, 2H), 7.72-7.59 (m, 4H), 7.34-7.20 (m, 2H), 2.53 (s, 3H).

### Synthesis of BB-9

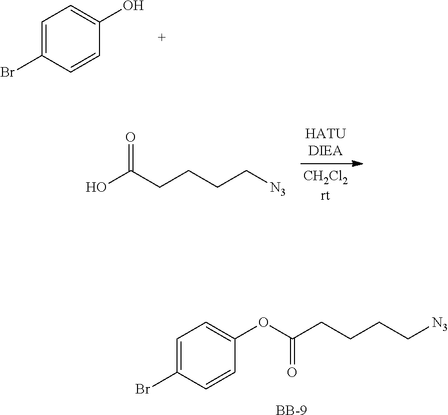 [SMILES: Oc1ccc(Br)cc1 ; [N-]=[N+]=NCCCCC(=O)O ; [N-]=[N+]=NCCCCC(=O)Oc1ccc(Br)cc1 ; "BB-9"]

A 100 mL round-bottom flask equipped with a stir bar was charged with 4-bromophenol (314 mg, 1.82 mmol), followed by the addition of 8 mL of dioxane to provide a clear solution. Next, 5-azidopentanoic acid (200 mg, 1.40 mmol), DIEA (0.973 μL, 5.59 mmol), and HATU (797 mg, 2.10 mmol) were added, and the resulting mixture was stirred at rt for 48 hours. The resulting mixture was quenched with saturated NaHCO₃ solution (100 mL) and partitioned between CH₂Cl₂ (200 mL) and water (100 mL). The organic phase was collected, and the aqueous phase was extracted with CH₂Cl₂ (100 mL) twice. The combined organic extract was washed with brine (50 mL), dried over sodium sulfate, filtered, and concentrated under reduced pressure. The obtained residue was purified by column chromatography (using Biotage® Sfär Silica 60 μm 25 g column, 95:5 to 3:1 hexane:ethyl acetate) and dried under reduced pressure, yielding the desired compound as a clear oil (338 mg, 81%).

¹H NMR (400 MHz, CDCl3): δ 7.49 (d, J=8.8 Hz, 2H), 6.97 (d, J=8.8 Hz, 2H), 3.35 (t, J=6.6 Hz, 2H), 2.60 (t, J=7.3 Hz, 2H), 1.90-1.78 (m, 2H), 1.77-1.67 (m, 2H).

¹³C NMR (101 MHz, CDCl₃): δ 171.41, 149.74, 132.59, 123.44, 119.02, 51.13, 33.78, 28.34, 22.13.

HRMS (DART) m/z calcd for C₁₁H₁₃O₂N₃Br ([M+H]⁺) 298.0186, found 298.0183.

### Synthesis of BB-10

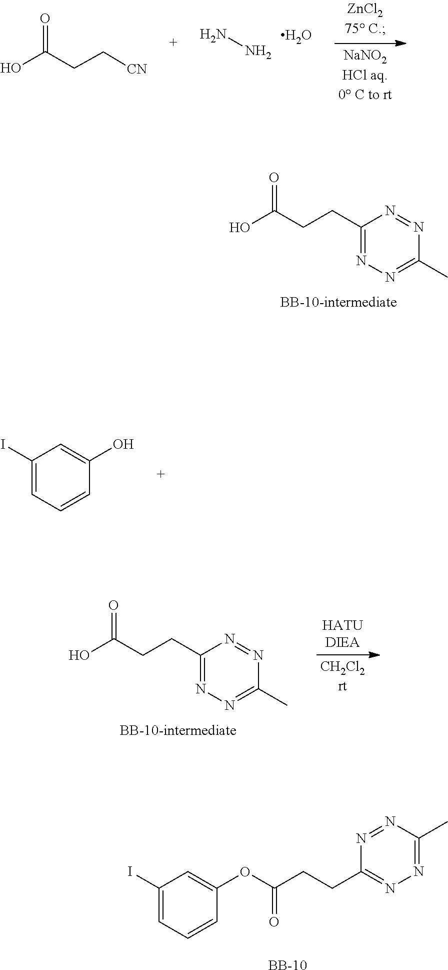

The BB-10-intermediate was synthesized as previously described (WO2014065860). The 1H NMR spectra of the obtained material were identical to those reported in the literature.

A 20 mL scintillation glass vial equipped with a stir bar was charged with 3-iodophenol (45.4 mg, 0.309 mmol), followed by the addition of 2 mL of CH₂Cl₂ to provide a clear solution. Next, BB-10-intermediate (40.0 mg, 0.238 mmol), DIEA (104 μL, 0.952 mmol), and HATU (90.5 mg, 0.357 mmol) were added, and the resulting mixture was stirred at rt for 12 hours. The resulting mixture was directly purified by column chromatography (using Biotage® Sfär Silica 60 μm 10 g column, 80:20 to 1:1 hexane:ethyl acetate) and dried under reduced pressure, yielding the desired compound as a pink oil (50 mg, 57%).

¹H NMR (400 MHZ, CDCl3): δ 7.57 (ddd, J=6.0, 3.1, 1.6 Hz, 1H), 7.50-7.44 (m, 1H), 7.14-7.05 (m, 2H), 3.75 (t, J=6.9 Hz, 2H), 3.31 (t, J=6.9 Hz, 2H), 3.06 (s, 3H).

### Synthesis of BB-11

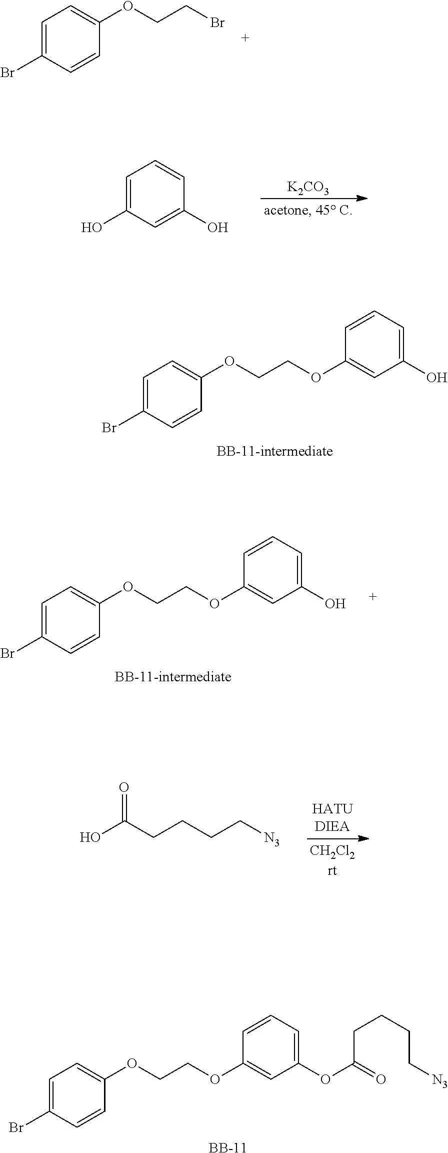

A 50 mL round-bottom flask equipped with a stir bar was charged with 1-bromo-4-(2-bromoethoxy)benzene (600 mg, 2.14 mmol), followed by the addition of 10 mL of acetone to provide a clear solution. Next, resorcinol (1.18 g, 10.7 mmol) and K₂CO₃ (1.48 g, 10.7 mmol) were added, and the resulting mixture was stirred at 45° C. for 12 hours. The resulting mixture was cooled to room temperature, followed by filtration using a Büchner funnel. The collected solid was then washed once with acetone (5 mL), and the resulting solution was concentrated under reduced pressure. The obtained residue was purified by column chromatography (using Biotage® Sfär Silica 60 μm 25 g column, 95:5 to 3:1 hexane:ethyl acetate) and dried under reduced pressure, yielding the BB-11-intermediate as a clear oil (415 mg, 63%).

¹H NMR (400 MHZ, CDCl₃): δ 7.39 (d, J=9.0 Hz, 2H), 7.14 (t, J=8.5 Hz, 1H), 6.83 (d, J=9.0 Hz, 2H), 6.57-6.49 (m, 1H), 6.47-6.43 (m, 2H), 4.71 (s, 1H), 4.28 (s, 4H).

¹³C NMR (101 MHZ, CDCl₃): δ 160.05, 157.90, 156.85, 132.46, 130.38, 129.66, 116.68, 113.49, 108.43, 108.06, 107.22, 102.49, 66.89, 66.57.

A 20 mL scintillation glass vial equipped with a stir bar was charged with BB-11-intermediate (150 mg, 0.485 mmol), followed by the addition of 2 mL of CH₂Cl₂ to provide a clear solution. Next, 5-azidopentanoic acid (63.1 mg, 0.441 mmol), DIEA (300 μL, 1.76 mmol), and HATU (252 mg, 0.662 mmol) were added, and the resulting mixture was stirred at rt for 12 hours. The resulting mixture was directly purified by column chromatography (using Biotage® Sfär Silica 60 μm 10 g column, 80:20 to 1:1 hexane:ethyl acetate) and dried under reduced pressure, yielding the desired compound as a pale pink solid (116 mg, 61%).

¹H NMR (400 MHZ, CDCl₃): δ 7.39 (d, J=9.0 Hz, 2H), 7.31-7.26 (m, 1H), 6.87-6.78 (m, 3H), 6.74-6.66 (m, 2H), 4.29 (s, 4H), 3.35 (t, J=6.7 Hz, 2H), 2.60 (t, J=7.2 Hz, 2H), 1.91-1.79 (m, 2H), 1.78-1.67 (m, 2H).

### Synthesis of BB-12

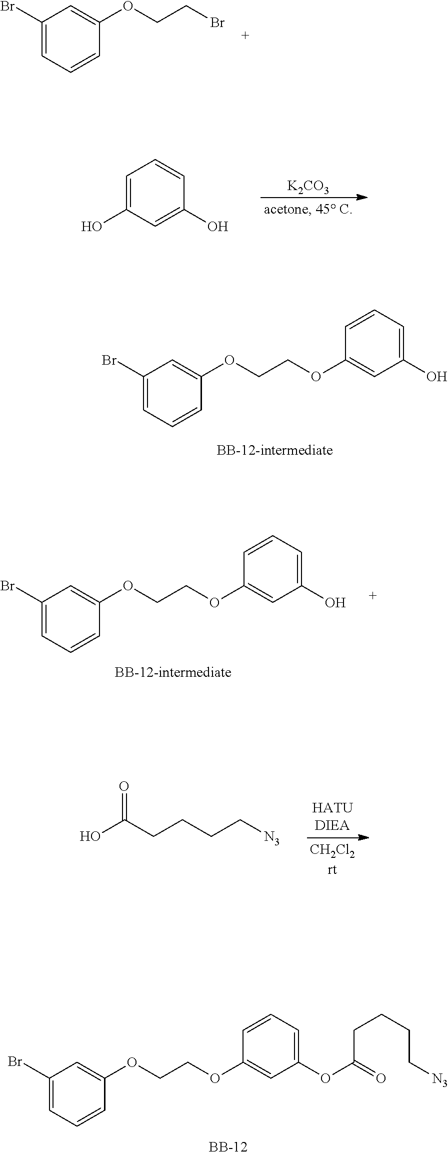

A 50 mL round-bottom flask equipped with a stir bar was charged with 1-bromo-3-(2-bromoethoxy)benzene (600 mg, 2.14 mmol), followed by the addition of 10 mL of acetone to provide a clear solution. Next, resorcinol (1.18 g, 10.7 mmol) and K₂CO₃ (1.48 g, 10.7 mmol) were added, and the resulting mixture was stirred at 45° C. for 12 hours. The resulting mixture was cooled to room temperature, followed by filtration using a Büchner funnel. The collected solid was then washed once with acetone (5 mL), and the resulting solution was concentrated under reduced pressure. The obtained residue was purified by column chromatography (using Biotage® Sfär Silica 60 μm 25 g column, 95:5 to 3:1 hexane:ethyl acetate) and dried under reduced pressure, yielding BB-12-intermediate as a clear oil (415 mg, 63%).

¹H NMR (400 MHZ, CDCl₃): δ 7.20-7.07 (m, 4H), 6.91-6.86 (m, 1H), 6.53 (ddd, J=8.3, 2.3, 1.0 Hz, 1H), 6.48-6.42 (m, 2H), 4.72 (s, 1H), 4.29 (s, 4H).

¹³C NMR (101 MHZ, CDCl₃): δ 160.01, 159.52, 156.86, 130.72, 130.37, 124.38, 122.95, 118.13, 113.89, 108.44, 107.18, 102.49, 66.85, 66.50.

HRMS (ESI) m/z calcd for C₁₄H₁₄O₃Br ([M+H]⁺) 309.0126, found 309.0133.

A 20 mL scintillation glass vial equipped with a stir bar was charged with BB-12-intermediate (75 mg, 0.242 mmol), followed by the addition of 2 mL of CH₂Cl₂ to provide a clear solution. Next, 5-azidopentanoic acid (52.0 mg, 0.363 mmol), DIEA (158 μL, 0.968 mmol), and HATU (184 mg, 0.484 mmol) were added, and the resulting mixture was stirred at rt for 12 hours. The resulting mixture was directly purified by column chromatography (using Biotage® Sfär Silica 60 μm 10 g column, 80:20 to 1:1 hexane:ethyl acetate) and dried under reduced pressure, yielding the desired compound as a pale pink solid (74 mg, 70%).

¹H NMR (400 MHZ, CDCl₃): δ 7.29 (t, J=8.1 Hz, 1H), 7.20-7.09 (m, 3H), 6.92-6.79 (m, 2H), 6.75-6.66 (m, 2H), 3.35 (t, J=6.7 Hz, 2H), 2.60 (t, J=7.2 Hz, 2H), 1.91-1.79 (m, 2H), 1.78-1.68 (m, 2H).

¹³C NMR (101 MHz, CDCl₃): δ 171.51, 159.40, 159.35, 151.58, 130.62, 129.95, 124.28, 122.83, 117.98, 114.29, 113.73, 112.28, 108.44, 66.64, 66.57, 51.06, 33.75, 28.26, 22.10.

HRMS (ESI) m/z calcd for C₁₉H₂₁O₄N₃Br ([M+H]⁺) 434.0715, found 434.0673.

### Synthesis of BB-13

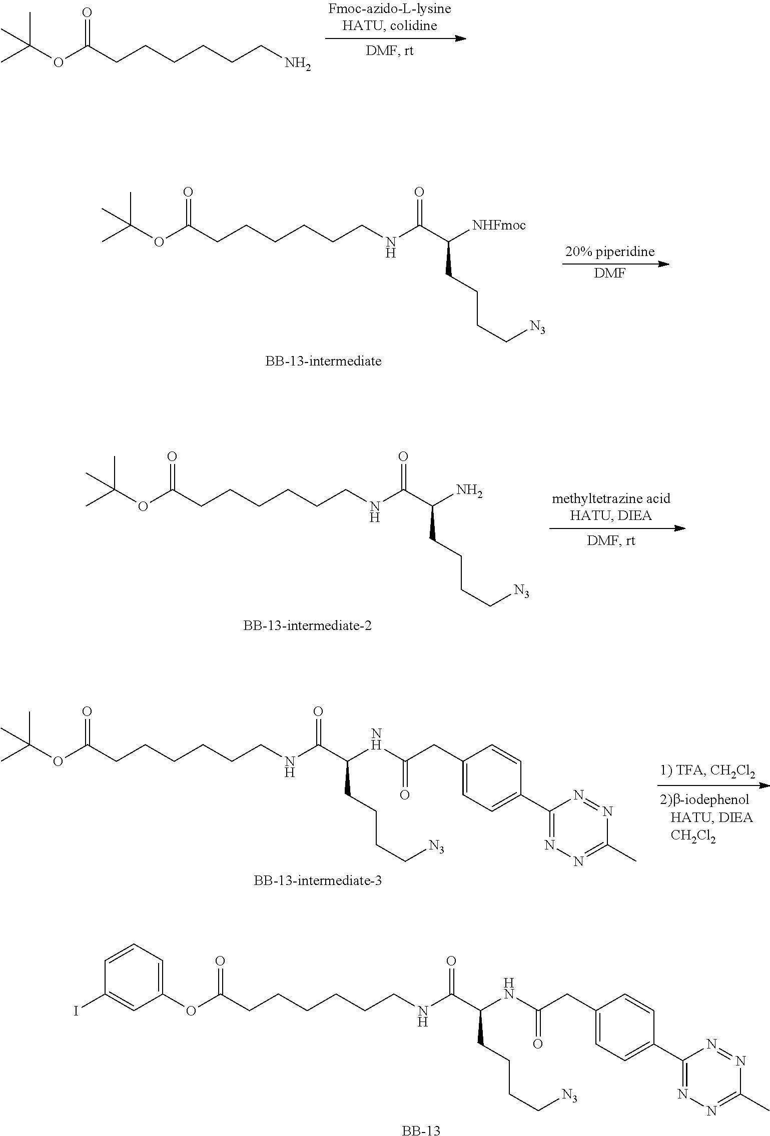

A 50 mL round-bottom flask equipped with a stir bar was charged with 7-amino-heptanoic acid t-butyl ester (346 mg, 1.72 mmol), followed by the addition of 9 mL of DMF to provide a clear solution. Next, Fmoc-L-azidolysine (746 mg, 1.89 mmol), 2,4,6-colidine (0.909 μL, 6.84 mmol), and HATU (781 mg, 2.06 mmol) were added, and the resulting mixture was stirred at rt for 12 hours. The resulting mixture was quenched with saturated NaHCO₃ solution (100 mL) and partitioned between ethyl acetate (200 mL) and water (100 mL). The organic phase was collected, and the aqueous phase was extracted with ethyl acetate (100 mL) twice. The combined organic extract was washed with saturated ammonium chloride solution (100 mL) twice and brine (50 mL), dried over sodium sulfate, filtered, and concentrated under reduced pressure. The obtained residue was purified by column chromatography (using Biotage® Sfär Silica 60 μm 25 g column, 95:5 to 2:1 hexane:ethyl acetate) and dried under reduced pressure, yielding BB-13-intermediate-1 as a clear oil (868 mg, 87%).

¹H NMR (400 MHZ, CDCl₃): δ 7.77 (d, J=7.5 Hz, 2H), 7.58 (d, J=7.5 Hz, 2H), 7.41 (t, J=7.5 Hz, 2H), 7.32 (t, J=7.4 Hz, 2H), 5.90 (s, 1H), 5.32 (d, J=8.3 Hz, 1H), 4.42 (s, 2H), 4.21 (t, J=6.7 Hz, 1H), 3.34-3.17 (m, 4H), 2.19 (t, J=7.4 Hz, 2H), 1.57 (s, 8H), 1.43 (s, 9H), 1.43-1.40 (m, 2H), 1.31 (s, 4H).

¹³C NMR (101 MHZ, CDCl₃): δ 173.28, 171.35, 156.33, 143.85, 141.45, 127.89, 127.21, 125.13, 120.14, 80.19, 67.13, 54.94, 51.26, 47.28, 39.62, 35.52, 32.38, 29.38, 28.69, 28.63, 28.24, 26.60, 24.99, 22.80.

HRMS (ESI) m/z calcd for C₃₂H₄₄O₅N₅ ([M+H]⁺) 578.3342, found 578.3315.

A 50 mL round-bottom flask equipped with a stir bar was charged with BB-13-intermediate-1 (400 mg, 0.69 mmol), followed by the addition of piperidine in DMF (3.4 mL, 20 v/v %) to provide a clear solution. The resulting mixture was stirred at rt for 2 hours. The resulting mixture was diluted with 20 mL of toluene and concentrated under reduced pressure. To remove DMF, 20 mL of toluene was added to the reaction mixture, and the solvent was subsequently evaporated under reduced pressure three times. The obtained residue was purified by column chromatography (using Biotage® Sfär Silica 60 μm 25 g column, 100:0 to 95:5 CH₂Cl₂+0.5 v/v % trimethylamine: MeOH) and dried under reduced pressure, yielding BB-13-intermediate-2 as a clear oil (162 mg, 66%).

¹H NMR (400 MHZ, CDCl₃): δ 7.30 (s, 1H), 3.43 (dd, J=7.7, 4.8 Hz, 1H), 3.34-3.19 (m, 4H), 2.20 (t, J=7.5 Hz, 2H), 2.11-1.69 (m, 4H), 1.69-1.44 (m, 8H), 1.44 (s, 9H), 1.33 (p, J=3.5 Hz, 4H).

¹³C NMR (101 MHZ, CDCl₃): δ 173.68, 173.41, 80.22, 54.89, 51.30, 39.22, 35.58, 34.12, 29.46, 28.79, 28.75, 28.24, 26.71, 25.05, 22.94.

HRMS (ESI) m/z calcd for C₁₇H₃₄O₃N₅ ([M+H]⁺) 356.2662, found 356.2644.

A 20 mL scintillation glass vial equipped with a stir bar was charged with BB-13-intermediate-2 (93 mg, 0.261 mmol), followed by the addition of 2 mL of CH₂Cl₂ to provide a clear solution. Next, methyltetrazine acid (50 mg, 0.217 mmol), DIEA (154 μL, 0.868 mmol), and HATU (124 mg, 0.326 mmol) were added, and the resulting mixture was stirred at rt for 12 hours. The resulting mixture was directly purified by column chromatography (using Biotage® Sfär Silica 60 μm 10 g column, 80:20 to 1:1 hexane:ethyl acetate) and dried under reduced pressure, yielding BB-13-intermediate-3 as a pale pink solid (101 mg, 82%).

¹H NMR (400 MHZ, CDCl₃): δ 8.58 (d, J=8.4 Hz, 2H), 7.50 (d, J=8.2 Hz, 2H), 6.19 (d, J=8.0 Hz, 1H), 5.98 (s, 1H), 4.36 (q, J=7.2 Hz, 1H), 3.68 (s, 2H), 3.23 (dq, J=7.3, 4.9 Hz, 4H), 3.10 (s, 3H), 2.19 (t, J=7.4 Hz, 2H), 1.90-1.77 (m, 1H), 1.66-1.41 (m, 7H), 1.44 (s, 9H), 1.40-1.24 (m, 6H).

¹³C NMR (101 MHz, CDCl₃): δ 173.24, 171.27, 170.37, 167.39, 163.89, 139.63, 130.95, 130.26, 128.43, 80.18, 53.21, 51.21, 43.46, 39.62, 35.49, 32.22, 29.32, 28.70, 28.63, 28.24, 26.62, 24.98, 22.76, 21.28.

HRMS (ESI) m/z calcd for C₂₈H₄₂O₄N₉ ([M+H]⁺) 568.3360, found 568.3334.

A 10 mL round-bottom flask equipped with a stir bar was charged with BB-13-intermediate-3 (30 mg, 0.053 mmol), followed by the addition of dichloromethane (2 mL) to provide a clear solution. Next, TFA (0.4 mL) was added, and the resulting mixture was stirred at rt for 3 hours. The reaction mixture was concentrated under reduced pressure, and the resulting residue was used in the next step without further purification.

Dichloromethane (1 mL) was added to a 10 mL round-bottom flask containing the residue obtained from the previous reaction, resulting in a clear solution. Next, 3-iodophenol (17.5 mg, 0.080 mmol), DIEA (55.0 μL, 0.318 mmol), and HATU (34.3 mg, 0.090 mmol) were added, and the resulting mixture was stirred at rt for 12 hours. The resulting mixture was directly purified by column chromatography (using Biotage® Sfär Silica 60 μm 10 g column, 80:20 to 1:1 hexane:ethyl acetate v/v) and dried under reduced pressure, yielding BB-13 as a pale pink solid (35.9 mg, 95%).

1H NMR (400 MHZ, CDCl₃): δ 8.58 (d, J=8.1 Hz, 2H), 7.56 (d, J=7.5 Hz, 1H), 7.53-7.42 (m, 3H), 7.14-7.02 (m, 2H), 6.15 (d, J=8.0 Hz, 1H), 5.97 (t, J=6.0 Hz, 1H), 4.35 (q, J=7.2 Hz, 1H), 3.67 (s, 2H), 3.23 (dq, J=7.0, 4.5 Hz, 4H), 3.09 (s, 3H), 2.80 (s, 1H), 2.53 (t, J=7.4 Hz, 2H), 1.84 (dq, J=14.2, 7.3 Hz, 1H), 1.72 (p, J=7.3 Hz, 2H), 1.65-1.55 (m, 2H), 1.56-1.45 (m, 3H), 1.47-1.23 (m, 6H).

¹³C NMR (101 MHz, CDCl₃): δ 171.84, 171.25, 170.38, 167.43, 163.90, 151.08, 139.55, 135.04, 131.03, 130.90, 130.82, 130.27, 128.50, 121.30, 93.65, 53.26, 51.22, 43.53, 39.57, 38.75, 34.18, 32.11, 29.79, 29.34, 28.70, 28.62, 26.56, 24.73, 22.79, 21.30.

HRMS (ESI) m/z calcd for C₃₀H₃₇O₄N₉I ([M+H]⁺) 714.2013, found 714.2008.

### Synthesis of Palladium Oxidative Addition Complexes

Synthesis of [(cod)Pd(CH₂TMS)₂] (cod=1,5 cyclooctadiene)

This compound was synthesized as previously described (28). The ¹H and ¹³C NMR spectra of the obtained material were identical to those reported in the literature.

Synthesis of sodium 2′-dicyclohexylphosphino-2,6-dimethoxy-1,1′-biphenyl-3-sulfonate hydrate (sSPhos)

This compound was synthesized as previously described (43). The ¹H and ¹³C NMR spectra of the obtained material were identical to those reported in the literature.

### Synthesis of BB-14

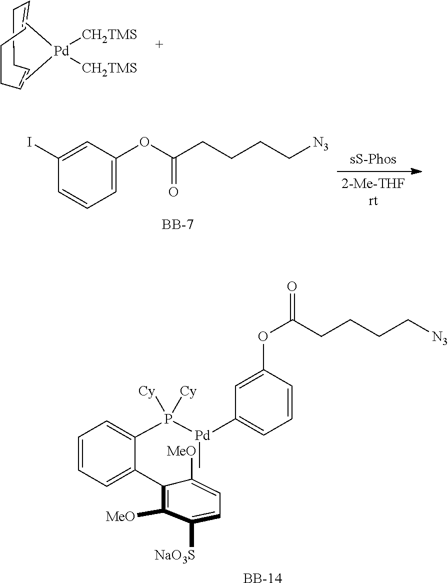

A 5 mL scintillation glass vial equipped with a stir bar was charged with BB-7 (58.8 mg, 0.171 mmol) and sSPhos (82.8 mg, 0.162 mmol), and 2-methyltetrahydrofuran (0.51 mL). The reaction solution was sonicated for 1 min until it became completely clear, and then the [(cod)Pd(CH₂TMS)₂] (60.0 mg, 0.154 mmol) was added, and the resulting mixture was stirred at rt for 2 hours. After the reaction time, pentane (1 mL) was added to the reaction mixture. The vial containing the white suspension was placed in a centrifuge and centrifuged at 3220 rcf for 2 min. The cap was removed, and the supernatant was decanted. The stir bar was removed, and the solid was resuspended in 2-methyltetrahydrofuran (1.0 mL) and pentane (1.0 mL). The vial was capped and sonicated for 1 min to obtain a homogeneous suspension. The vial was then centrifuged again at 3220 rcf for 2 min. The cap was removed, and the supernatant was decanted. This sonication/centrifugation/decanting procedure was repeated twice. The resulting beige solid was dried under reduced pressure to give the desired crude product as a light brown solid (111 mg, 75%). The obtained crude product was used in the following reaction without further purification.

MS (m/z): C₃₇H₄₇N₃O₇PPdS⁺ ([M−I−Na+H]⁺) 814.2, found 814.1.

### Synthesis of BB-15

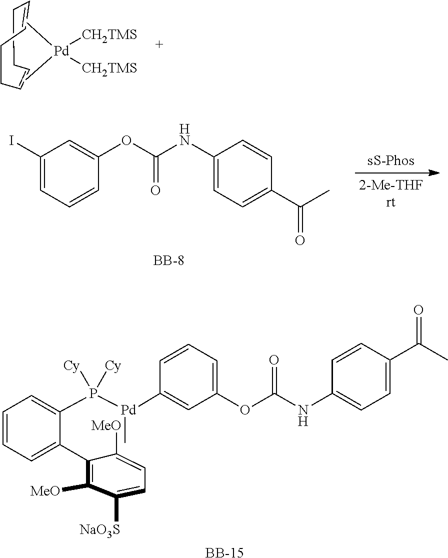

This compound was synthesized following the general procedure described in the section on the synthesis of BB-14. BB-8 (16.3 mg, 0.043 mmol), sSPhos (20.7 mg, 0.040 mmol), [(cod)Pd(CH₂TMS)₂] (15.0 mg, 0.039 mmol), and 2-methyltetrahydrofuran (0.5 mL) were used, and BB-15 was obtained as a light brown solid (39 mg, quantitative).

MS (m/z): C₄₁H₄₇NO₈PPdS⁺ ([M−I−Na+H]⁺) 850.2, found 850.1.

### Synthesis of BB-16

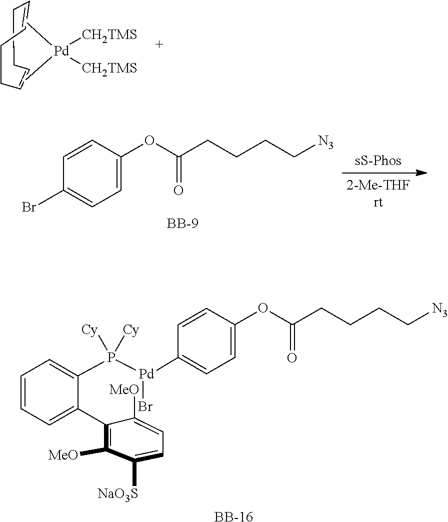

This compound was synthesized following the general procedure described in the section on the synthesis of BB-14. BB-9 (12.7 mg, 0.043 mmol), sSPhos (20.7 mg, 0.040 mmol), [(cod)Pd(CH₂TMS)2] (15.0 mg, 0.039 mmol), and 2-methyltetrahydrofuran (0.5 mL) were used, and BB-16 was obtained as a light brown solid (34 mg, 96%).

MS (m/z): C₃₇H₄₇NO₇PPdS⁺ ([M−N₂−Br−Na+H]⁺) 786.2, found 786.1.

### Synthesis of BB-17

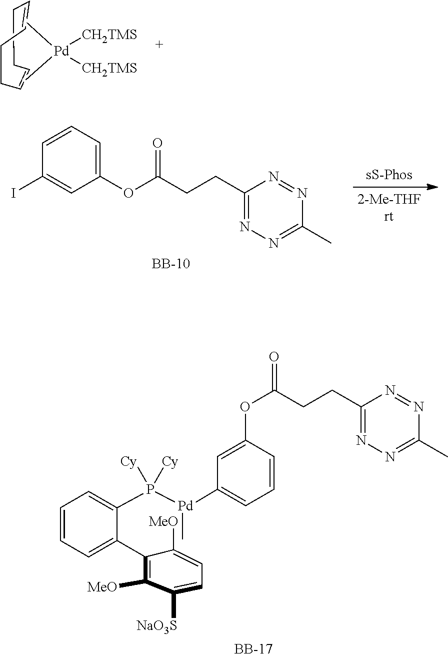

This compound was synthesized following the general procedure described in the section on the synthesis of BB-14. BB-10 (15.8 mg, 0.043 mmol), sSPhos (20.7 mg, 0.040 mmol), [(cod)Pd(CH₂TMS)₂] (15.0 mg, 0.039 mmol), and 2-methyltetrahydrofuran (0.5 mL) were used, and BB-17 was obtained as a light brown solid (21 mg, 55%).

MS (m/z): C₃₈H₄₆N₄O₇PPdS⁺ ([M−I−Na+H]⁺) 839.2, found 839.1.

### Synthesis of BB-18

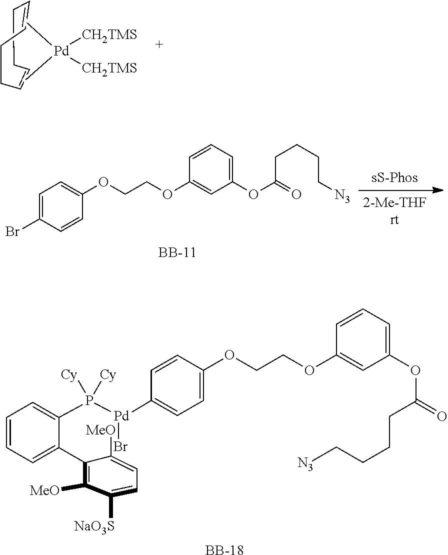

This compound was synthesized following the general procedure described in the section on the synthesis of BB-14. BB-11 (18.5 mg, 0.043 mmol), sSPhos (20.7 mg, 0.040 mmol), [(cod)Pd(CH₂TMS)₂] (15.0 mg, 0.039 mmol), and 2-methyltetrahydrofuran (0.5 mL) were used, and BB-18 was obtained as a light brown solid (32 mg, 80%).

MS (m/z): C₄₅H₅₅N₃O₉PPdS⁺ ([M−Br−Na+H]⁺) 950.0, found 950.1.

### Synthesis of BB-19

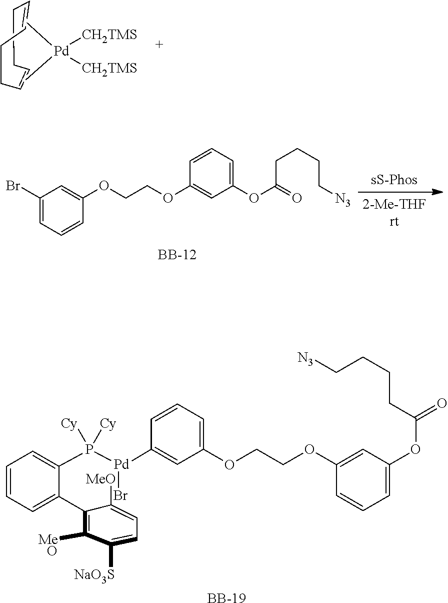

This compound was synthesized following the general procedure described in the section on the synthesis of BB-14. BB-12 (17.0 mg, 0.039 mmol), sSPhos (19.0 mg, 0.037 mmol), [(cod)Pd(CH₂TMS)₂] (13.8 mg, 0.035 mmol), and 2-methyltetrahydrofuran (0.5 mL) were used, and BB-19 was obtained as a light brown solid (26 mg, 71%).

MS (m/z): C₄₅H₅₅N₃O₉PPdS⁺ ([M−Br−Na+H]⁺) 950.0, found 950.1.

### Synthesis of BB-20

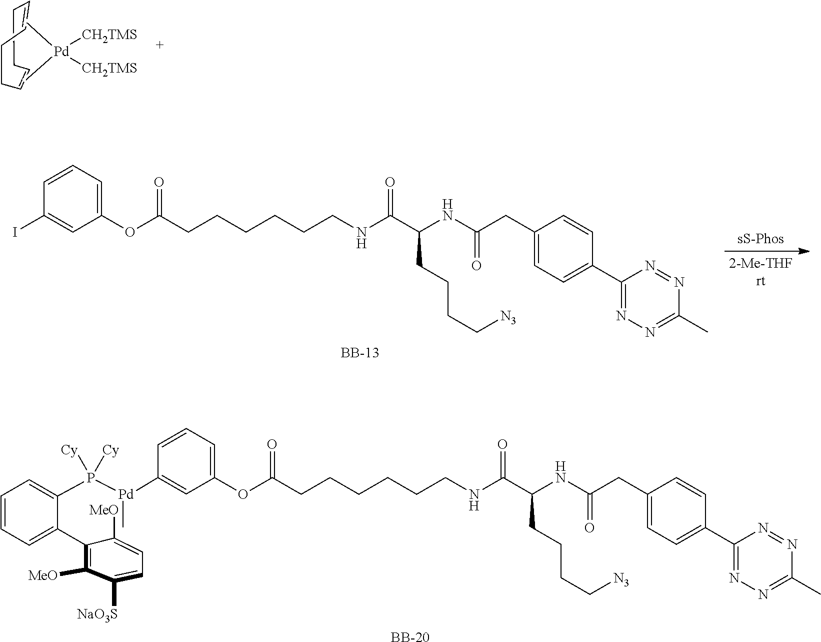

This compound was synthesized following the general procedure described in the section on the synthesis of BB-14. BB-13 (15.0 mg, 0.021 mmol), sSPhos (10.3 mg, 0.020 mmol), [(cod)Pd(CH₂TMS)₂] (7.5 mg, 0.019 mmol), and 2-methyltetrahydrofuran (0.4 mL) were used, and BB-20 was obtained as a light brown solid (22 mg, 85%).

MS (m/z): C₅₆H₇₁N₉O₉PPdS⁺ ([M−I−Na+H]⁺) 1182.0, found 1182.0.

### Synthesis of Electrophile-Attached Z33 Reagents

Synthesized electrophile-attached Z33 reagents are listed in Table 16.

Each modified peptide was synthesized as follows:

### Synthesis of N₃-1-I

1 (15 mg) was weighed into a 1.5 mL microcentrifuge tube, and DMF (200 μL) was added. Additionally, BB-14 (7.1 mg) was weighed into a 0.6 mL microcentrifuge tube, and DMF (0.18 mL) was added to prepare a separate brown solution using sonication. The BB-14 solution was added to the solution of 1, and the mixture was vortexed briefly and allowed to stand at room temperature for 30 min. Then the reaction mixture was transferred to a 50 mL centrifuge tube and diluted to 5 mL with water. Subsequently, the reaction solution was directly loaded onto a Biotage® Sfär C18 D column (12 g) and purified using the following conditions: 5 CV 5% B, 1 CV 5-10% B, 20 CV 10-35% B. 1 μL of a 20-fold diluted solution of the obtained fractions was analyzed by LC-MS, and the fractions containing only the target product were collected and lyophilized to obtain N₃-1-I (6.7 mg, 45%) as a white solid.

### Synthesis of N₃-2-I

2 (15.0 mg) and BB-14 (7.1 mg) were used to synthesize N₃-2-I following the same procedure as for N₃-1-I. N₃-2-I (6.7 mg, 42%) was obtained as a white solid.

### Synthesis of N₃-3-I

3 (15.7 mg) and BB-14 (6.7 mg) were used to synthesize N₃-3-I following the same procedure as for N₃-1-I. N₃-3-I (9.5 mg, 58%) was obtained as a white solid.

### Synthesis of N₃-4-I

4 (5.0 mg) and BB-14 (2.3 mg) were used to synthesize N₃-4-I following the same procedure as for N₃-1-I. N₃-4-I was obtained as a white solid.

### Synthesis of N₃-5-I

5 (5.0 mg) and BB-14 (2.3 mg) were used to synthesize N₃-5-I following the same procedure as for N₃-1-I. N₃-5-I was obtained as a white solid.

### Synthesis of N₃-6-I

6 (5.0 mg) and BB-14 (2.3 mg) were used to synthesize N₃-6-I following the same procedure as for N₃-1-I. N₃-6-I was obtained as a white solid.

### Synthesis of BB-21

2 (5.0 mg) and BB-15 (2.5 mg) were used to synthesize BB-21 following the same procedure as for N₃-1-I. BB-21 (2.5 mg, 47%) was obtained as a white solid.

### Synthesis of BB-22

2 (5.0 mg) and BB-16 (2.2 mg) were used to synthesize BB-22 following the same procedure as for N₃-1-I. BB-22 (3.4 mg, 65%) was obtained as a white solid.

### Synthesis of BB-23

2 (5.0 mg) and BB-17 (2.4 mg) were used to synthesize BB-23 following the same procedure as for N₃-1-I. BB-23 (1.2 mg, 23%) was obtained as a white solid.

### Synthesis of N₃-12-VIII

2 (5.0 mg) and BB-20 (3.3 mg) were used to synthesize N₃-12-VIII following the same procedure as for N₃-1-I. N₃-12-VIII (0.32 mg, 6%) was obtained as a white solid.

### Synthesis of N₃-7-VI

BB-2 (5.0 mg) and BB-18 (2.6 mg) were used to synthesize N₃-7-VI following the same procedure as for N₃-1-I. N₃-7-VI (3.2 mg, 59%) was obtained as a white solid.

### Synthesis of N₃-8-VI

BB-3 (5.0 mg) and BB-18 (2.6 mg) were used to synthesize N₃-8-VI following the same procedure as for N₃-1-I. N₃-8-VI (3.1 mg, 57%) was obtained as a white solid.

### Synthesis of N₃-7-VII

BB-2 (5.0 mg) and BB-19 (2.6 mg) were used to synthesize N₃-7-VII following the same procedure as for N₃-1-I. N₃-7-VII (3.2 mg, 59%) was obtained as a white solid.

### Synthesis of N₃-8-VII

BB-3 (5.0 mg) and BB-19 (2.6 mg) were used to synthesize N₃-8-VII following the same procedure as for N₃-1-I. N₃-8-VII (4.5 mg, 83%) was obtained as a white solid.

### Synthesis of N₃-9-VII

BB-4 (5.0 mg) and BB-19 (2.6 mg) were used to synthesize N₃-9-VII following the same procedure as for N₃-1-I. N₃-9-VII (3.5 mg, 64%) was obtained as a white solid.

### Synthesis of N₃-10-VII

BB-5 (5.0 mg) and BB-19 (2.6 mg) were used to synthesize N₃-10-VII following the same procedure as for N₃-1-I. N₃-10-VII (0.5 mg, 9%) was obtained as a white solid.

### Synthesis of N₃-11-VII

BB-6 (5.0 mg) and BB-19 (2.6 mg) were used to synthesize N₃-11-VII following the same procedure as for N₃-1-I. N₃-11-VII (0.48 mg, 9%) was obtained as a white solid.

Click handle transfer to IgGs using electrophile-Z33 variants.

### Bioconjugation to Trastuzumab (IgG1) (See FIG. 79A)

### General Reaction Procedure

Reactions were performed on a 20 μL scale using 0.2 mL PCR tubes. First, the electrophile-attached Z33 peptide reagent was dissolved in water to prepare a 2 mg/mL solution. Trastuzumab (MedChemExpress LLC. or Bio X Cell), electrophile-attached Z33 peptide reagent (2 mg/mL), HEPES (pH 8.5), and NaCl were added to achieve final concentrations of 10 μM, 100 μM, 100 mM, and 200 mM respectively. The contents were mixed using a 10 μL pipette, followed by incubation at room temperature in the dark for 24 hours. The resulting reaction solution was quenched with a 100 mM glycine buffer or 100 mM Tris buffer and analyzed by LC-MS.

### General Procedure of LC-MS Analysis

A reaction solution corresponding to 9.0 μg of IgG was taken into a PCR tube and diluted with Tris buffer (100 mM, pH 8.1) to a final volume of 6 μL. Separately, a mixed solution of PNGase F (New England BioLabs, 63 units) and Glycoprotein Denaturing Buffer (New England Biolabs, 1×, 4 μL) was prepared and added to the reaction solution, followed by incubation at 37° C. for 4 to 12 hours. DTT (200 mM in water, 2 μL) was added, and the mixture was further incubated at 37° C. for 1 hour. Finally, the solution was diluted to a volume of 40 μL with a mixture of water and acetonitrile (95:5), and 8 μL of the diluted solution was injected into LC-MS.

### Procedure Using Reagent BB-23 or N₃-12-VIII

Reactions were performed on a 20 μL scale using 0.2 mL PCR tubes. First, the electrophile-attached Z33 peptide reagent was dissolved in water to prepare a 2 mg/mL solution. Trastuzumab (Bio X Cell), electrophile-attached Z33 peptide reagent (2 mg/mL), HEPES (pH 8.5), and NaCl were added to achieve final concentrations of 10 μM, 100 μM, 100 mM, and 200 mM respectively. The contents were mixed using a 10 μL pipette, followed by incubation at 37° C. in the dark for 24 hours. 4 hours after the start of incubation, an additional 10 equivalent of electrophile-attached Z33 peptide was added (200 μM in total). The resulting reaction solution was quenched with a 100 mM glycine buffer or 100 mM Tris buffer and analyzed by LC-MS. Bioconjugation to other IgGs (human IgG1,2,4 & mouse IgG1) (See FIG. 81A)

Reactions were performed on a 10 μL scale using 0.2 mL PCR tubes. First, the electrophile-attached Z33 peptide reagent was dissolved in water to prepare a 2 mg/mL solution. Denosumab (Invitrogen), dupilumab (Invitrogen), or mouse IgG1 (Invitrogen, subtype controlled)), electrophile-attached Z33 peptide reagent (2 mg/mL), HEPES buffer (pH 8.5), and NaCl solution were added to achieve final concentrations of 5 μM, 100 μM, 100 mM, and 200 mM. The contents were mixed using a 5 μL pipette and incubated at room temperature in the dark for 24 hours. The resulting reaction solution was quenched with a 100 mM Tris buffer and analyzed by LC-MS. Sample preparation for LC-MS analysis was performed in the same manner as described in the “Bioconjugation to Trastuzumab (IgG1) (See FIG. 79A)” section.

### Determination of the Modification Sites

The modified trastuzumab (30 μg) from entry 6 of 5.2.1 was diluted to 36 μL using Tris buffer (50 mM, pH 8.0). PNGase F (New England BioLabs, 200 units) was added to the solution and incubated at 37° C. for 4 hours. The reaction solution was then brought to room temperature, and urea solution (6 M in 50 mM Tris pH 8.0, 14 μL) and DTT solution (200 mM, 1.5 L) were added and incubated at 37° C. for 1 hour. After that, iodoacetamide (800 mM, 1 μL) was added and incubated at room temperature for 30 min. The resulting solution was diluted 2-fold using Tris buffer (50 mM, pH 8.0), and then Trypsin/Lys-C mix (Promega, 0.2 μg/μL in solution, 6 μL) was added and incubated at 37° C. for 18 hours. The reaction solution was then purified by pipetting with Ziptip, lyophilized, and analyzed by nLC-MS/MS (using nLC-MS/MS method B). Obtained raw data were analyzed using Thermo Scientific FreeStyle™ 1.6. De novo sequencing was performed with PEAKS 8.5 with the following search parameters: Parent Mass Error Tolerance=15.0 ppm; Fragment Mass Error Tolerance=0.02 Da, Enzyme=None; modification setting are listed in the table below; Max Variable PTM Per Peptide=10; Report #Peptides=10. Sequencing results were exported as .csv reports for all de novo candidates. Finally, fragments corresponding to trastuzumab sequences were manually searched from the .csv file, and the main modification sites were estimated using each fragment's peak area.

| | Amino | Molecular | |
|---|---|---|---|
| PTM type | acids | weight | Setting Reason |
| Fixed | C | 57.0214 | Capped by iodoacetamide |
| Variable | K | 125.0589 | Modified by the electrophile |
| | | | pattached eptide reagents |
| Variable | M | 15.9949 | Unintentional natural oxidation |

*Since the mass of the modified lysine is the same as the mass of Pro-Arg (271.1644), both possibilities were considered in the fragment search.
   Expanding the Chemistry with Other Z33 Variants

### Using the Electrophile-Attached Z33M3C/Hcy Reagents (See, FIG. 97A)

Reactions were performed on a 20 μL scale using 0.2 mL PCR tubes. First, the electrophile-attached Z33 peptide reagent was dissolved in water to prepare a 2 mg/mL solution. Trastuzumab (Bio X Cell), electrophile-attached Z33 peptide reagent (2 mg/mL), HEPES buffer (pH 8.5), and NaCl solution were added to achieve final concentrations of 10 μM, 200 μM, 100 mM, and 200 mM. The contents were mixed using a 10 μL pipette, followed by incubation at room temperature or 37° C. in the dark for 24 hours. The resulting reaction solution was quenched with a 100 mM Tris buffer and analyzed by LC-MS. Sample preparation for LC-MS analysis was performed in the same manner as described in the “Bioconjugation to Trastuzumab (IgG1) (See FIG. 79A)” section.

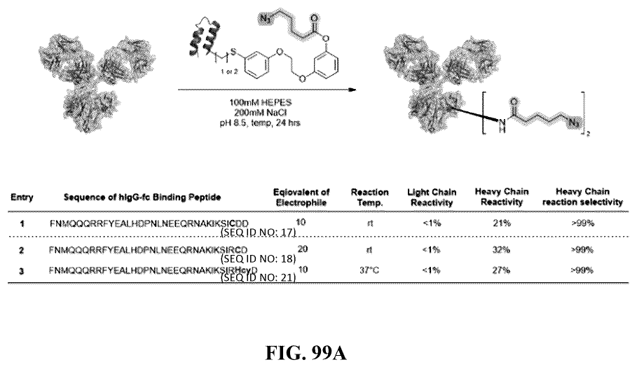

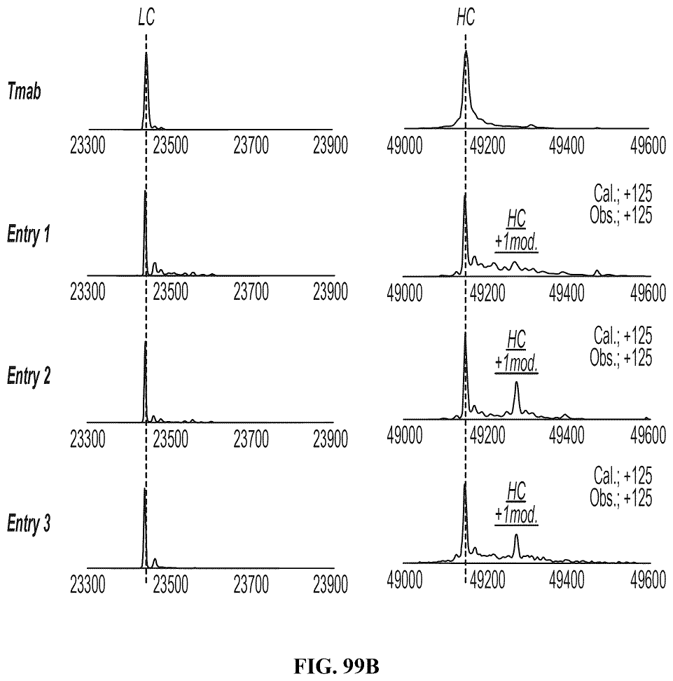

### Using the Electrophile-Attached Z33R31C/Hcy and D32C/Hcy Reagents (See, FIG. 99A)

Reactions were performed on a 20 μL scale using 0.2 mL PCR tubes. First, the electrophile-attached Z33 peptide reagent was dissolved in water to prepare a 2 mg/mL solution. Trastuzumab (Bio X Cell), electrophile-attached Z33 peptide reagent (2 mg/mL), HEPES buffer (pH 8.5), and NaCl solution were added to achieve final concentrations of 10 μM, 200 μM, 100 mM, and 200 mM. The contents were mixed using a 10 μL pipette, followed by incubation at room temperature or 37° C. in the dark for 24 hours. The resulting reaction solution was quenched with a 100 mM Tris buffer and analyzed by LC-MS. Sample preparation for LC-MS analysis was performed in the same manner as described in the “Bioconjugation to Trastuzumab (IgG1) (See FIG. 79A)” section.

### Dual Modification of the Trastuzumab Heavy Chain by Stepwise Reactions

First Tmab modification reaction was performed on a 200 μL scale using 1.5 mL microcentrifuge tube. N₃-2-I was dissolved in water to prepare a 2 mg/mL solution. Trastuzumab (Bio X Cell), N₃-2-I (2 mg/mL), HEPES buffer (pH 8.5), NaCl solution were added to achieve final concentrations of 10 μM, 100 μM, 100 mM, and 200 mM. The contents were mixed using a 200 μL pipette and incubated at room temperature for 24 hours. The resulting reaction solution was transferred to an Amicon 30K MWCO filter, and 200 μL of a 100 mM citric acid (pH 2.7) was added. The solvent was removed by centrifugation, and 200 μL of a 50 mM Tris buffer (pH 8.0) was added. Another round of centrifugation, 30 μL of a reaction mixture was obtained. Sample preparation for LC-MS analysis was performed in the same manner as described in the “Bioconjugation to Trastuzumab (IgG1) (See FIG. 79A)” section. The concentration of the modified Tmab was determined to be 2.2 mg/mL by measuring absorbance at 280 nm.

Second Tmab modification was performed on a 20 μL scale using 0.2 mL PCR tubes. N₃-8-VI was dissolved in water to prepare a 2 mg/mL solution. Modified Tmab above, N₃-8-VI (2 mg/mL), HEPES buffer (pH 8.5), NaCl solution were added to achieve final concentrations of 10 μM, 100 μM, 100 mM, and 200 mM. The contents were mixed using a 20 μL pipette and incubated at 37° C. for 24 hours. The resulting reaction solution was quenched with a 100 mM Tris buffer and analyzed by LC-MS. Sample preparation for LC-MS analysis was performed in the same manner as described in the “Bioconjugation to Trastuzumab (IgG1) (See FIG. 79A)” section.

### Trastuzumab Modification in the Presence of RNase A

Reactions were performed on a 20 μL scale using 0.2 mL PCR tubes. First, N₃-2-I was dissolved in water to prepare a 2 mg/mL solution. Tmab (Bio X Cell), RNAse A (VWR), N₃-2-I (2 mg/mL), and PBS were added to achieve final concentrations of 10 μM, 10 μM, 200 μM, and 1×. The contents were mixed using a 5 μL pipette and incubated at 37° C. for 24 hours. The resulting reaction solution was transferred to an Amicon 10K MWCO filter, and 200 μL of a 100 mM citric acid (pH 2.7) was added. The solvent was removed by centrifugation, and 200 μL of a 100 mM Tris buffer (pH 8.1) was added. Another round of centrifugation, 30 μL of a reaction mixture was obtained. Sample preparation for LC-MS analysis was performed in the same manner as described in the “Bioconjugation to Trastuzumab (IgG1) (See FIG. 79A)” section.

Antibody-Drug Conjugate (ADC) Preparation with MMAE Transfer Reagent

Reactions were performed on a 20 μL scale using 0.2 mL PCR tubes. First, N₃-2-I and DBCO-PEG4-Val-Cit-PAB-MMAE (BROADPHARM) were dissolved to prepare 2 mg/mL (in water) and 2 mM (in DMSO) solutions, respectively. A mixture of HEPES buffer and NaCl solution or PBS was added to the tubes, followed by N₃-2-I (2 mg/mL, 4.29 μL) and DBCO-PEG4-Val-Cit-PAB-MMAE (2 mM, 1.1 μL) were added, mixed using a 10 μL pipette, and incubated at 4° C. for 30 min. The final concentrations of HEPES buffer (pH 8.5), NaCl solution, and PBS buffer were 100 mM, 200 mM and 1×, respectively. Tmab (Bio X Cell, 9.2 mg/mL, 1.61 μL) was then added to the reaction solution and incubated at room temperature or 37° C. for hours. The resulting reaction solution was quenched with 100 mM Tris buffer and analyzed by SDS-PAGE.

### Sample Preparation for SDS-PAGE Analysis

The reaction mixture corresponding to 3.5 μg of IgG was taken to a PCR tube, and diluted with Tris buffer (100 mM, pH 8.1) to a final volume of 3 μL. Then, Laemmli sample buffer (BIO-RAD, 2×+5% 2-mercaptoethanol, 3 μL) was added, and the mixture was heated at 70° C. for 5 min. After returning the reaction solution to room temperature, 2 μL was loaded onto an SDS-Gel. Subsequently, electrophoresis, washing, staining, and analysis were performed using the methods described in General Information Section.

### Animal Experiments

All animal experiments were performed under an MIT institute approved IACUC protocol following federal, state, and local guidelines for the care and use of animals (protocol number #0821-058-24). The studies were performed either on 35-45 g (12-16 weeks-old) wild-type female Swiss mice, 25-30 g (14-20 weeks-old) male and female C57BL/6J mice, or 55-70 g (16 weeks-old) male and female obese Lep^{ob/ob} mice, all purchased from The Jackson Laboratory (MA, USA). Mice were housed with free access to normal food diet and water ad libitum unless stated otherwise for the need of the experiment. For subcutaneous (SC) injections, the mice were shortly anesthetized beforehand with 2-3% isoflurane along with O₂, then shaved on the right flank. Intravenous injections (IV) were performed through IV catheterization; the mice were anesthetized with 2-3% isoflurane along with O₂, then a catheter was inserted in the lateral tail vein to ensure the injection was properly done. Intraperitoneal injections (IP) were performed on non-anesthetized mice, in the lower right abdominal quadrant. Blood collection for the ELISA assays and biodistributions was performed as a terminal procedure by heart puncture immediately after the sacrifice. For Ip-GTT and Ip-ITT mice were restrained and gently pressed at the tail following tail pricks. The animals were sacrificed by CO₂ inhalation followed by cervical dislocation. All compounds injected in mice were either USP grade, sterile, or filtered using 0.22 μm.

Mouse IgG Painting with Azido Moieties

### Enzyme-Linked Immunosorbent Assay (ELISA)

Two sandwich ELISA assays have been developed in-house, one for the detection of the azido moiety conjugated mouse IgG (mIgG, E1), the other one for the detection of the whole mIgGs (modified and unmodified) in the sample (E2). Both E1 and E2 were run simultaneously on the same plate, using the same initial stock sample, and were processed in parallel at the same time for every step. The same azido-free capture goat anti-mouse polyclonal antibody (Creative Diagnostics) was used for E1-E2 assays at 1 μg/mL (100 μL per well) for incubation overnight at 4° C. Washing steps were performed thoroughly between each incubation by using 0.02% Tween 20-PBS. Saturation step (1 h, RT) and sample dilutions were all carried in 5% milk-PBS. For E1, the detection was performed using 1:150 dilution of DBCO-PEG4-Biotin (Jena Bioscience) (in 5% milk-PBS) while for E2 the detection was made using 1:2000 azido-free biotin-conjugated donkey anti-mouse polyclonal antibody (Creative Diagnostics) in 5% milk-PBS, both for 150 min at RT, in the dark. A dilution of 1:500 HRP-conjugated streptavidin (Thermo Fisher Scientific Inc.) in 5% milk-PBS was then added for 1 h, RT, in the dark. TMB substrate (Thermo Fisher Scientific Inc.) was added (100 μL per well, RT) simultaneously in E1 and E2 wells, and the reaction was stopped with 2N H₂SO₄ stop solution (Thermo Fisher Scientific Inc.) after 5 to 10 min. Absorbances were measured at 450 nm and 540 nm (A₀=A₄₅₀ₙₘ−A₅₄₀ₙₘ) using a Spark microplate reader (Tecan, USA). The background noise (A_{blank}) from the serum was subtracted to get the final absorbance of the sample (Aₛₐₘₚₗₑ=A₀−A_{blank}). The ratio Aₛₐₘₚₗₑ (E1)/Asample (E2) was calculated to estimate the percentage of azido transfer on the total amount of IgG in the sample.

### Qualitative Assessment of Azido-Transfer to Mouse IgG in Serum

Reactions were performed on a 40 μL scale using 0.2 mL PCR tubes. First, the azido electrophile-attached Z33 peptide reagent 1, 2 or 3 was dissolved in water to prepare a 2 mg/ml solution. Mouse serum (Invitrogen, S/N 24-5544-94, 31.2 μL) and electrophile-attached Z33 peptide reagent (2 mg/mL, 8.8 μL) were added to the PCR tubes to a final concentration of 100 mM of electrophile-attached Z33 reagent. The contents were mixed using a 10 μL pipette, followed by incubation at 37° C. in the dark for 2, 6, or 24 hours. To remove the excess amount of the remaining electrophile-attached Z33 reagent, the reaction mixture was filtered using an Amicon® filter. The reaction mixture was diluted to 200 μL with citrate buffer (100 mM, pH 2.7) and filtered through an Amicon® filter (0.5 mL, 30KMWCO) at 14000 rcf for 7 min. After filtration, 400 μL of PBS buffer was added and centrifuged again. Finally, the remaining solution (roughly 20 μL) on the Amicon® filter was transferred to a microcentrifuge tube. Obtained samples were then diluted 1:100 to 1:2000 in 5% milk-PBS for dosage using E1/E2 assays.

### Qualitative Assessment of Azido-Transfer to Mouse IgG In Vivo

Female Swiss mice (n=3 per group) were either IP or SC injected with 10 mg/kg (˜90 nmol) to 30 mg/kg (˜280 nmol) of N₃-1, N₃-2, or N₃-3 (diluted in 1×PBS). Blood was recovered by intracardiac puncture (terminal procedure) at 24 hours post injection, then was centrifuged 10 min at 15,000 rcf to get serum. Obtained samples were then diluted 1:2 in 5% milk-PBS for dosage using E1/E2 assays.

Mouse IgG Painting with Radionuclides
Preparation of Deferoxamine B-Attached Z33 Reagents and Radiolabeling with Zirconium-89

First, 1, 2, 3 were dissolved in HEPES buffer (100 mM in ultra-trace elemental water; Fisher Scientific, pH 6.7) to prepare a 2 mg/mL solution of each. Deferoxamine-DBCO (obtained from Macrocycles, 1 mM in DMSO, 11.1 μL) and 1, 2, 3 (360 μL) solutions were then added to the tube, stirred gently by hand, and the tube was placed on ice for 1 hour.

The obtained samples were incubated with 37 MBq of [⁸⁹Zr]Zr-oxalate (obtained from Mallinckrodt Institute of Radiology, Nuclear Pharmacy Cyclotron; dilution in 0.5 M HEPES, pH 6.7) for 1 hour in the wet ice under mild agitation. Purification was then performed using a Zeba spin column (0.5 mL, 7K MWCO) to remove the non-complexed [⁸⁹Zr]Zr-oxalate, for 5 min at 1,200 rcf. Instant-thin layer chromatography (iTLC) was performed to verify the success of the radiolabeling with elution in 0.1 M citrate buffer.

### REFERENCES FOR EXAMPLE 14

1. B. M. Cooper, J. Iegre, D. H. O′ Donovan, M. Ölwegård Halvarsson, D. R. Spring, Peptides as a platform for targeted therapeutics for cancer: peptide-drug conjugates (PDCs). Chem. Soc. Rev. 50, 1480-1494 (2021).
2. D. Agyei, I. Ahmed, Z. Akram, H. M. N. Iqbal, M. K. Danquah, Protein and peptide biopharmaceuticals: an overview. PPL 24, 94-101 (2017).
3. L. Wang, N. Wang, W. Zhang, X. Cheng, Z. Yan, G. Shao, X. Wang, R. Wang, C. Fu, Therapeutic peptides: current applications and future directions. Sig Transduct Target Ther 7, 48 (2022).
4. A. Rondon, S. Mahri, F. Morales-Yanez, M. Dumoulin, R. Vanbever, Protein engineering strategies for improved pharmacokinetics. Advanced Functional Materials 31, 2101633 (2021).
5. H. E. Blackwell, R. H. Grubbs, Highly efficient synthesis of covalently cross-linked peptide helices by ring-closing metathesis. Angewandte Chemie International Edition 37, 3281-3284 (1998).
6. C. Heinis, T. Rutherford, S. Freund, G. Winter, Phage-encoded combinatorial chemical libraries based on bicyclic peptides. Nat Chem Biol 5, 502-507 (2009).
7. D. J. Drucker, Advances in oral peptide therapeutics. Nat Rev Drug Discov 19, 277-289 (2020).
8. S. Ansari, B. Khoo, T. Tan, Targeting the incretin system in obesity and type 2 diabetes mellitus. Nat Rev Endocrinol, doi: 10.1038/s41574-024-00979-9 (2024).
9. A. K. Chetty, E. Rafi, N. J. Bellini, N. Buchholz, D. Isaacs, A review of incretin therapies approved and in late-stage development for overweight and obesity management. Endocrine Practice 30, 292-303 (2024).
10. G. Cantini, E. Mannucci, M. Luconi, Perspectives in GLP-1 research: new targets, new receptors. Trends in Endocrinology & Metabolism 27, 427-438 (2016).
11. F. B. Ahmad, J. A. Cisewski, J. Xu, R. N. Anderson, Provisional mortality data—United States, 2022. MMWR Morb. Mortal. Wkly. Rep. 72, 488-492 (2023).
12. A. Moiz, J. Y. Levett, K. B. Filion, K. Peri, P. Reynier, M. J. Eisenberg, Long-term efficacy and safety of once-weekly Semaglutide for weight loss in patients without diabetes: a systematic review and meta-analysis of randomized controlled trials. The American Journal of Cardiology, S0002914924003199 (2024).
13. A. M. Chao, J. S. Tronieri, A. Amaro, T. A. Wadden, Semaglutide for the treatment of obesity. Trends in Cardiovascular Medicine 33, 159-166 (2023).
14. M. Davies, L. Færch, O. K. Jeppesen, A. Pakseresht, S. D. Pedersen, L. Perreault, J. Rosenstock, I. Shimomura, A. Viljoen, T. A. Wadden, I. Lingvay, Semaglutide 2.4 mg once a week in adults with overweight or obesity, and type 2 diabetes (STEP 2): a randomised, double-blind, double-dummy, placebo-controlled, phase 3 trial. The Lancet 397, 971-984 (2021).
15. M. M. Véniant, S.-C. Lu, L. Atangan, R. Komorowski, S. Stanislaus, Y. Cheng, B. Wu, J. R. Falsey, T. Hager, V. A. Thomas, M. Ambhaikar, L. Sharpsten, Y. Zhu, V. Kurra, R. Jeswani, R. K. Oberoi, J. R. Parnes, N. Honarpour, J. Neutel, J. L. Strande, A GIPR antagonist conjugated to GLP-1 analogues promotes weight loss with improved metabolic parameters in preclinical and phase 1 settings. Nat Metab 6, 290-303 (2024).
16. R. Hammoud, D. J. Drucker, Beyond the pancreas: contrasting cardiometabolic actions of GIP and GLP1. Nat Rev Endocrinol 19, 201-216 (2023).
17. F. W. R. Brambell, The transmission of immunity from mother to young and the catabolism of immunoglobulins. The Lancet 288, 1087-1093 (1966).
18. M. Pyzik, L. K. Kozicky, A. K. Gandhi, R. S. Blumberg, The therapeutic age of the neonatal Fc receptor. Nat Rev Immunol 23, 415-432 (2023).
19. M. H. Packard, J. H. Cox, V. P. Suding, J. J. Topczewski, The effect of proximal functionality on the allylic azide equilibrium. Eur J Org Chem 2017, 6365-6368 (2017).
20. N. Hartrampf, A. Saebi, M. Poskus, Z. P. Gates, A. J. Callahan, A. E. Cowfer, S. Hanna, S. Antilla, C. K. Schissel, A. J. Quartararo, X. Ye, A. J. Mijalis, M. D. Simon, A. Loas, S. Liu, C. Jessen, T. E. Nielsen, B. L. Pentelute, Synthesis of proteins by automated flow chemistry. Science 368, 980-987 (2020).
21. A. C. Braisted, J. A. Wells, Minimizing a binding domain from protein A. Proc. Natl. Acad. Sci. U.S.A. 93, 5688-5692 (1996).
22. M. D. Simon, Y. Maki, A. A. Vinogradov, C. Zhang, H. Yu, Y.-S. Lin, Y. Kajihara, B. L. Pentelute, D-Amino Acid Scan of Two Small Proteins. J. Am. Chem. Soc. 138, 12099-12111 (2016).
23. G. M. Edelman, B. A. Cunningham, W. E. Gall, P. D. Gottlieb, U. Rutishauser, M. J. Waxdal, The covalent structure of an entire γG immunoglobulin molecule. Proc. Natl. Acad. Sci. U.S.A. 63, 78-85 (1969).
24. M. Dondelinger, P. Filée, E. Sauvage, B. Quinting, S. Muyldermans, M. Galleni, M. S. Vandevenne, Understanding the significance and implications of antibody numbering and antigen-binding surface/residue definition. Front. Immunol. 9, 2278 (2018).
25. C. Yu, J. Tang, A. Loredo, Y. Chen, S. Y. Jung, A. Jain, A. Gordon, H. Xiao, Proximity-induced site-specific antibody conjugation. Bioconjugate Chem. 29, 3522-3526 (2018).
26. K. Yamada, N. Shikida, K. Shimbo, Y. Ito, Z. Khedri, Y. Matsuda, B. A. Mendelsohn, AJICAP: affinity peptide mediated regiodivergent functionalization of native antibodies. Angew Chem Int Ed 58, 5592-5597 (2019).
27. T. Fujii, Y. Matsuda, T. Seki, N. Shikida, Y. Iwai, Y. Ooba, K. Takahashi, M. Isokawa, S. Kawaguchi, N. Hatada, T. Watanabe, R. Takasugi, A. Nakayama, K. Shimbo, B. A. Mendelsohn, T. Okuzumi, K. Yamada, AJICAP Second generation: improved chemical site-specific conjugation technology for antibody-drug conjugate production. Bioconjugate Chem., acs.bioconjchem.3c00040 (2023).
28. E. V. Vinogradova, C. Zhang, A. M. Spokoyny, B. L. Pentelute, S. L. Buchwald, Organometallic palladium reagents for cysteine bioconjugation. Nature 526, 687-691 (2015).
29. A.-S. Klein-Schneegans, L. Kuntz, P. Fonteneau, F. Loor, Serum concentrations of IgM, IgG1, IgG2b, IgG3 and IgA in C57BL6 mice and their congenics at the lpr (lymphoproliferation) locus. Journal of Autoimmunity 2, 869-875 (1989).
30. H. O. Sarvas, I. J. T. Seppälä, T. Tähtinen, F. Péterfy, O. Mäkelä, Mouse IgG antibodies have subclass associated affinity differences. Molecular Immunology 20, 239-246 (1983).
31. E. M. Sletten, C. R. Bertozzi, From Mechanism to Mouse: A Tale of Two Bioorthogonal Reactions. Acc. Chem. Res. 44, 666-676 (2011).
32. S. Latvala, B. Jacobsen, M. B. Otteneder, A. Herrmann, S. Kronenberg, Distribution of FcRn across species and tissues. J Histochem Cytochem. 65, 321-333 (2017).
33. T. Li, J. P. Balthasar, FcRn expression in wildtype mice, transgenic mice, and in human tissues. Biomolecules 8, 115 (2018).
34. G. W. Severin, J. W. Engle, T. E. Barnhart, R. J. Nickles, ⁸⁹Zr radiochemistry for positron emission tomography. MC 7, 389-394 (2011).
35. D. G. Brown, H. J. Wobst, A decade of FDA-approved drugs (2010-2019): trends and future directions. J. Med. Chem. 64, 2312-2338 (2021).
36. A. Elmaleh-Sachs, J. L. Schwartz, C. T. Bramante, J. M. Nicklas, K. A. Gudzune, M. Jay, Obesity management in adults: a review. JAMA 330, 2000 (2023).
37. J. Andraos, H. Muhar, S. R. Smith, Beyond glycemia: comparing tirzepatide to GLP-1 analogues. Rev Endocr Metab Disord, doi: 10.1007/s11154-023-09825-1 (2023).
38. F. Suriano, S. Vieira-Silva, G. Falony, M. Roumain, A. Paquot, R. Pelicaen, M. Régnier, N. M. Delzenne, J. Raes, G. G. Muccioli, M. Van Hul, P. D. Cani, Novel insights into the genetically obese (ob/ob) and diabetic (db/db) mice: two sides of the same coin. Microbiome 9, 147 (2021).
39. E. Rentzeperi, S. Pegiou, T. Koufakis, M. Grammatiki, K. Kotsa, Sex differences in response to treatment with glucagon-like peptide 1 receptor agonists: opportunities for a tailored approach to diabetes and obesity care. JPM 12, 454 (2022).
40. J. Lau, P. Bloch, L. Schäffer, I. Pettersson, J. Spetzler, J. Kofoed, K. Madsen, L. B. Knudsen, J. McGuire, D. B. Steensgaard, H. M. Strauss, D. X. Gram, S. M. Knudsen, F. S. Nielsen, P. Thygesen, S. Reedtz-Runge, T. Kruse, Discovery of the once-weekly glucagon-like peptide-1 (GLP-1) analogue Semaglutide. J. Med. Chem. 58, 7370-7380 (2015).
41. S. Gabery, C. G. Salinas, S. J. Paulsen, J. Ahnfelt-Rønne, T. Alanentalo, A. F. Baquero, S. T. Buckley, E. Farkas, C. Fekete, K. S. Frederiksen, H. C. C. Helms, J. F. Jeppesen, L. M. John, C. Pyke, J. Nøhr, T. T. Lu, J. Polex-Wolf, V. Prevot, K. Raun, L. Simonsen, G. Sun, A. Szilvásy-Szabó, H. Willenbrock, A. Secher, L. B. Knudsen, Semaglutide lowers body weight in rodents via distributed neural pathways. JCI Insight 5, e133429 (2020).
42. R. M. Gutgesell, R. Nogueiras, M. H. Tschöp, T. D. Müller, Dual and triple incretin-based co-agonists: novel therapeutics for obesity and diabetes. Diabetes Ther 15, 1069-1084 (2024).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A first modified affinity peptide, wherein:
n1 instances of the amino acid residues of the affinity peptide are independently modified with a moiety of Formula A′:
-L¹-E²-L²-(E³)ₙ₃ (A′);
n1 is 1, 2, or 3;
each instance of L¹ is independently substituted or unsubstituted, C₁₋₁₈ heteroalkylene, substituted or unsubstituted, C₁₋₁₈ alkylene, substituted or unsubstituted, C₂₋₁₈ alkenylene, substituted or unsubstituted, C₂₋₁₈ alkynylene, substituted or unsubstituted, C₂₋₁₈ heteroalkenylene, substituted or unsubstituted, C₂₋₁₈ heteroalkynylene, substituted or unsubstituted arylene, substituted or unsubstituted carbocyclylene, substituted or unsubstituted heterocyclylene, or substituted or unsubstituted heteroarylene, optionally wherein one, two, or three backbone atoms of the C₁₋₁₈ heteroalkylene, C₁₋₁₈ alkylene, C₂₋₁₈ alkenylene, C₂₋₁₈ alkynylene, C₂₋₁₈ heteroalkenylene, or C₂₋₁₈ heteroalkynylene are independently replaced with substituted or unsubstituted arylene, substituted or unsubstituted carbocyclylene, substituted or unsubstituted heterocyclylene, or substituted or unsubstituted heteroarylene, as valency permits;
each instance of E² is independently —O—C(═O)—, —O—S(═O)—, —O—S(═O)₂—, —O—C(═O)—NR^{a}—, —O—S(═O)—NR^{a}—, —O—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of n3 is independently 1, 2, 3, or 4; and
each instance of E³ is independently a first reactive moiety.

2. The first modified affinity peptide of claim 1, wherein Formula A′ is Formula A:
-L¹-E²-L²-E³ (A).

3. A second modified affinity peptide, wherein:
n1 instances of the amino acid residues of the affinity peptide are independently modified with a moiety of Formula B′:
-L¹-E²-L²-(E³⁴-L³-M)ₙ₃ (B′);
wherein:
n1 is 1, 2, or 3;
each instance of L¹ is independently substituted or unsubstituted, C₁₋₁₈ heteroalkylene, substituted or unsubstituted, C₁₋₁₈ alkylene, substituted or unsubstituted, C₂₋₁₈ alkenylene, substituted or unsubstituted, C₂₋₁₈ alkynylene, substituted or unsubstituted, C₂₋₁₈ heteroalkenylene, substituted or unsubstituted, C₂₋₁₈ heteroalkynylene, substituted or unsubstituted arylene, substituted or unsubstituted carbocyclylene, substituted or unsubstituted heterocyclylene, or substituted or unsubstituted heteroarylene, optionally wherein one, two, or three backbone atoms of the C₁₋₁₈ heteroalkylene, C₁₋₁₈ alkylene, C₂₋₁₈ alkenylene, C₂₋₁₈ alkynylene, C₂₋₁₈ heteroalkenylene, or C₂₋₁₈ heteroalkynylene are independently replaced with substituted or unsubstituted arylene, substituted or unsubstituted carbocyclylene, substituted or unsubstituted heterocyclylene, or substituted or unsubstituted heteroarylene, as valency permits;
each instance of E² is independently —O—C(═O)—, —O—S(═O)—, —O—S(═O)₂—, —O—C(═O)—NR^{a}—, —O—S(═O)—NR^{a}—, —O—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of n3 is independently 1, 2, 3, or 4;
each instance of E³⁴ is independently a moiety formed by reacting E³ with E⁴;
each instance of E³ is independently a first reactive moiety;
each instance of E⁴ is independently a second reactive moiety;
each instance of the first reactive moiety is capable of reacting with each instance of the second reactive moiety;
each instance of L³ is independently substituted or unsubstituted, C₁₋₂₀₀ heteroalkylene, substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkenylene, substituted or unsubstituted, C₂₋₂₀₀ heteroalkynylene, substituted or unsubstituted heterocyclylene, substituted or unsubstituted arylene, substituted or unsubstituted heteroarylene, substituted or unsubstituted carbocyclylene, or a bond, optionally wherein one or more backbone atoms of the C₁₋₂₀₀ heteroalkylene, C₁₋₂₀₀ alkylene, C₂₋₂₀₀ alkenylene, C₂₋₂₀₀ alkynylene, C₂₋₂₀₀ heteroalkenylene, or C₂₋₂₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits; and
each instance of M is independently a radical of a pharmaceutical agent or absent.

4. The second modified affinity peptide of claim 3, wherein Formula B′ is Formula B:
-L¹-E²-L²-E³⁴-L³-M (B).

5-17. (canceled)

18. An antibody-pharmaceutical agent conjugate, wherein:
n2 instances of the lysine residues of the antibody are independently modified with a moiety of Formula C′:
—(CH₂)₄-E¹²-L²-(E³⁴-L³-M)ₙ₃ (C′);
n2 is 1, 2, 3, 4, 5, or 6;
each instance of E¹² is independently —NH—C(═O)—, —NH—S(═O)—, —NH—S(═O)₂—, —NH—C(═O)—NR^{a}—, —NH—S(═O)—NR^{a}—, or —NH—S(═O)₂—NR^{a}—;
each instance of R^{a} is independently hydrogen, substituted or unsubstituted, C₁₋₆ alkyl, or a nitrogen protecting group;
each instance of L² is independently substituted or unsubstituted, C₁₋₁₀₀ alkylene, substituted or unsubstituted, C₂₋₁₀₀ alkenylene, substituted or unsubstituted, C₂₋₁₀₀ alkynylene, substituted or unsubstituted, C₁₋₁₀₀ heteroalkylene, substituted or unsubstituted, C₂₋₁₀₀ heteroalkenylene, substituted or unsubstituted, C₂₋₁₀₀ heteroalkynylene, substituted or unsubstituted heterocyclylene, substituted or unsubstituted arylene, substituted or unsubstituted heteroarylene, or substituted or unsubstituted carbocyclylene, optionally wherein one or more backbone atoms of the C₁₋₁₀₀ alkylene, C₂₋₁₀₀ alkenylene, C₂₋₁₀₀ alkynylene, C₁₋₁₀₀ heteroalkylene, C₂₋₁₀₀ heteroalkenylene, or C₂₋₁₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits;
each instance of n3 is independently 1, 2, 3, or 4;
each instance of E³⁴ is independently a moiety formed by reacting E³ with E⁴;
each instance of E³ is independently a first reactive moiety;
each instance of E⁴ is independently a second reactive moiety;
each instance of the first reactive moiety is capable of reacting with each instance of the second reactive moiety;
each instance of L³ is independently substituted or unsubstituted, C₁₋₂₀₀ heteroalkylene, substituted or unsubstituted, C₁₋₂₀₀ alkylene, substituted or unsubstituted, C₂₋₂₀₀ alkenylene, substituted or unsubstituted, C₂₋₂₀₀ alkynylene, substituted or unsubstituted, C₂₋₂₀₀ heteroalkenylene, substituted or unsubstituted, C₂₋₂₀₀ heteroalkynylene, substituted or unsubstituted heterocyclylene, substituted or unsubstituted arylene, substituted or unsubstituted heteroarylene, substituted or unsubstituted carbocyclylene, or a bond, optionally wherein one or more backbone atoms of the C₁₋₂₀₀ heteroalkylene, C₁₋₂₀₀ alkylene, C₂₋₂₀₀ alkenylene, C₂₋₂₀₀ alkynylene, C₂₋₂₀₀ heteroalkenylene, or C₂₋₂₀₀ heteroalkynylene are independently replaced with substituted or unsubstituted heterocyclylene, substituted or unsubstituted arylene, substituted or unsubstituted heteroarylene, or substituted or unsubstituted carbocyclylene, as valency permits; and
each instance of M is independently a radical of a pharmaceutical agent.

19. The antibody-pharmaceutical agent conjugate of claim 18, wherein Formula C′ is Formula C:
—(CH₂)₄-E¹²-L²-E³⁴-L³-M (C).

20-39. (canceled)

40. The first modified affinity peptide of claim 1, wherein at least one instance of E³ is a first click-chemistry handle.

41. The first modified affinity peptide of claim 1, wherein at least one instance of E³ is —N₃ or substituted or unsubstituted 1,2,4,5-tetrazinyl.

42-82. (canceled)

83. A composition comprising:
a first modified affinity peptide of claim 1; and
optionally one or more excipients.

84-85. (canceled)

86. A kit comprising:
a first modified affinity peptide of claim 1; and
instructions for using the first modified affinity peptide.

87. A method comprising administering to a subject in need thereof an effective amount of a first modified affinity peptide of claim 1.

88-97. (canceled)

98. A method comprising contacting a cell, tissue, or biological sample with a first modified affinity peptide of claim 1.

99-100. (canceled)

101. A composition comprising:
a second modified affinity peptide of claim 3; and
optionally one or more excipients.

102. A kit comprising:
a second modified affinity peptide of claim 3; and
instructions for using the second modified affinity peptide.

103. A method comprising administering to a subject in need thereof an effective amount of a second modified affinity peptide of claim 3.

104. A method comprising contacting a cell, tissue, or biological sample with a second modified affinity peptide of claim 3.

105. A composition comprising:
an antibody-pharmaceutical agent conjugate of claim 18; and
optionally one or more excipients.

106. A kit comprising:
an antibody-pharmaceutical agent conjugate of claim 18; and
instructions for using the antibody-pharmaceutical agent conjugate.

107. A method comprising administering to a subject in need thereof an effective amount of an antibody-pharmaceutical agent conjugate of claim 18.

108. A method comprising contacting a cell, tissue, or biological sample with an antibody-pharmaceutical agent conjugate of claim 18.


SEQUENCE LISTING (41 sequences)

SEQ ID NO: 1 - protein, 33 aa, synthetic construct
FNMQQQRRFYEALHDPNLNEEQRNAKIKSIRDD

SEQ ID NO: 2 - protein, 34 aa, synthetic construct
FNMQCQRRFYEALHDPNLNEEQRNAKIKSIRDDC

SEQ ID NO: 3 - protein, 13 aa, synthetic construct
DCAWHLGELVWCT

SEQ ID NO: 4 - protein, 17 aa, synthetic construct
RGNCAYHRGQLVWCTYH

SEQ ID NO: 5 - protein, 13 aa, synthetic construct
KMADPNRFRGKDL

SEQ ID NO: 6 - protein, 17 aa, synthetic construct
RGNCAYHRGKLVWCTYH

SEQ ID NO: 7 - protein, 58 aa, synthetic construct
ADNKFNKEQQNAFYEILHLPNLNEEQRNGFIQSLKDDPSQSANLLAEAKKLNDAQXXK

SEQ ID NO: 8 - protein, 17 aa, synthetic construct
RGNCAYHKGQLVWCTYH

SEQ ID NO: 9 - protein, 56 aa, synthetic construct
TTYKLVINGKTLKGETTTEAVDAATAEKVFKQYANDNGVDGEWTYDDATKTFTVTE

SEQ ID NO: 10 - protein, 17 aa, synthetic construct
GPDCAYHRGELVWCTFH

SEQ ID NO: 11 - protein, 34 aa, synthetic construct
CFNMQQQRRFYEALHDPNLNEEQRNAKIKSIRDD

SEQ ID NO: 12 - protein, 33 aa, synthetic construct
FNCQQQRRFYEALHDPNLNEEQRNAKIKSIRDD

SEQ ID NO: 13 - protein, 33 aa, synthetic construct
FNMQQQRRFYEALHDPCLNEEQRNAKIKSIRDD

SEQ ID NO: 14 - protein, 33 aa, synthetic construct
FNMQQQRRFYEALHDPNLNCEQRNAKIKSIRDD

SEQ ID NO: 15 - protein, 33 aa, synthetic construct
FNMQQQRRFYEALHDPNLNECQRNAKIKSIRDD

SEQ ID NO: 16 - protein, 33 aa, synthetic construct
FNMQQQRRFYEALHDPNLNEEQRCAKIKSIRDD

SEQ ID NO: 17 - protein, 33 aa, synthetic construct
FNMQQQRRFYEALHDPNLNEEQRNAKIKSICDD

SEQ ID NO: 18 - protein, 33 aa, synthetic construct
FNMQQQRRFYEALHDPNLNEEQRNAKIKSIRCD

SEQ ID NO: 19 - protein, 33 aa, synthetic construct; identical to SEQ ID NO: 14
  MOD_RES 20: note = homocysteine

SEQ ID NO: 20 - protein, 33 aa, synthetic construct; identical to SEQ ID NO: 14
  SITE 16: note = D-proline

SEQ ID NO: 21 - protein, 33 aa, synthetic construct; identical to SEQ ID NO: 18
  MOD_RES 32: note = homocysteine

SEQ ID NO: 22 - protein, 33 aa, synthetic construct; identical to SEQ ID NO: 12
  MOD_RES 3: note = homocysteine

SEQ ID NO: 23 - protein, 33 aa, synthetic construct; identical to SEQ ID NO: 12
  MOD_RES 3: note = homocysteine

SEQ ID NO: 24 - protein, 33 aa, synthetic construct; identical to SEQ ID NO: 18
  MOD_RES 32: note = homocysteine

SEQ ID NO: 25 - protein, 31 aa, synthetic construct
  MOD_RES 2: note = Aib
HXEGTFTSDVSSYLEGQAAKEFIAWLVRGRG

SEQ ID NO: 26 - protein, 31 aa, synthetic construct; identical to SEQ ID NO: 25
  MOD_RES 2: note = Aib

SEQ ID NO: 27 - protein, 33 aa, synthetic construct; identical to SEQ ID NO: 14
  MOD_RES 20: note = homocysteine
  SITE 33: note = D-Aspartic acid

SEQ ID NO: 28 - protein, 33 aa, synthetic construct; identical to SEQ ID NO: 14
  MOD_RES 20: note = homocysteine
  SITE 33: note = D-Aspartic acid
  SITE 1: note = D-phenylalanine

SEQ ID NO: 29 - protein, 33 aa, synthetic construct; identical to SEQ ID NO: 14
  MOD_RES 20: note = homocysteine
  SITE 1: note = D-phenylalanine
  SITE 32: note = D-Aspartic acid
  SITE 33: note = D-Aspartic acid

SEQ ID NO: 30 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 400 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPV

SEQ ID NO: 33 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICVNVHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 39 aa, synthetic construct
  MOD_RES 2: note = Aib
  MOD_RES 13: note = Aib
  MOD_RES 20: note = azidolysine
YXEGTFTSDYSIXLDKIAQKAFVQWLIAGGPSSGAPPPS

SEQ ID NO: 35 - protein, 31 aa, synthetic construct
HAEGTFTSDVSSYLEGQAAKEFIAWLVRGRG

SEQ ID NO: 36 - protein, 39 aa, synthetic construct; identical to SEQ ID NO: 34
  MOD_RES 2: note = Aib
  MOD_RES 13: note = Aib

SEQ ID NO: 37 - protein, 29 aa, synthetic construct
  MOD_RES 20: note = homocysteine
  SITE 16: note = D-proline
FNMQQQRRFYEALHDPNLNCEQRNAKIKS

SEQ ID NO: 38 - protein, 29 aa, synthetic construct
  MOD_RES 20: note = homocysteine
FNMQQQRRFYEALHDPNLNCEQRNAKIKS

SEQ ID NO: 39 - protein, 19 aa, synthetic construct
  MOD_RES 2: note = Aib
HXEGTFTSDVSSYLEGQAA

SEQ ID NO: 40 - protein, 11 aa, synthetic construct
EFIAWLVRGRG

SEQ ID NO: 41 - protein, 39 aa, synthetic construct; identical to SEQ ID NO: 34
  MOD_RES 2: note = Aib
  MOD_RES 13: note = Aib
  MOD_RES 20: note = azidolysine